(19) United States
(12) Patent Application Publication
(10) Pub. No.: US 2014/0213638 A1
(43) Pub. Date: Jul. 31, 2014

(54) Compositions and Biomarkers for Heart Disease, Injury and Failure and Methods of Use

(71) Applicant: Duke University, Durham, NC (US)

(72) Inventors: Dawn E. Bowles, Durham, NC (US); Carmelo A. Milano, Chapel Hill, NC (US); Valentino Piacentino, Oxford, CT (US); Matthew Schechter, Durham, NC (US)

(73) Assignee: Duke University, Durham, NC (US)

(21) Appl. No.: 14/165,429

(22) Filed: Jan. 27, 2014

Related U.S. Application Data
(60) Provisional application No. 61/756,521, filed on Jan. 25, 2013.

(51) Int. Cl. C07K 14/47

(52) U.S. Cl. CPC C07K 14/47
USPC 514/44 R

(57) ABSTRACT

The present disclosure provides biomarkers useful for determining the risk of, prognosis of, and/or diagnosis of conditions such as ischemic and/or non-ischemic heart failure in a subject.

### STATEMENT OF PRIORITY

This application claims the benefit, under 35 U.S.C. §119(e), of U.S. Provisional Application Ser. No. 61/756,521, filed Jan. 25, 2013, the entire contents of which are incorporated by reference herein.

### FIELD OF THE INVENTION

The present invention is directed to biomarkers and therapies for treatment of cardiac disorders, including heart disease, injury and failure.

### BACKGROUND OF THE INVENTION

Despite improved therapy and earlier diagnosis, heart failure (HF) continues to be a global health concern, with 5.7 million Americans diagnosed with HF in 2012.¹ Alarmingly, the lifetime risk of developing HF after age 40 is 20%, with the annual incidence approaching 10 per 1000 people after 65 years of age.² Although antihypertensive therapy and improved treatment for myocardial infarction have improved survival trends, more than half of all HF patients will still die within a 5-year period.^{3,4} Moreover, the increased urbanization of developing countries resulting in the adaptation of a sedentary lifestyle suggests that the worldwide incidence of HF will continue to rise.⁵

The treatment options for end-stage HF are limited and only include implantation of a ventricular assist device to mechanically unload the heart, heart transplantation, or palliation with continuous intravenous inotropic support. These options are also associated with high morbidity and mortality. Gene and cell therapy clinical trials suggest the potential to reverse failing heart phenotypes.^{6,7} Successful proof of principle clinical studies are encouraging, but further highlight the need to better define the molecules and biochemical pathways important to disease progression.

Ischemic and non-ischemic cardiomyopathies are two distinct types of heart disease that can lead to HF. Ischemic HF describes significantly impaired left ventricular function that results from reduced blood supply to the heart muscle, commonly from coronary artery disease. In contrast, non-ischemic HF has a range of etiologies, including congenital, infectious agents, autoimmune, and idiopathic causes. Identifying molecular differences between ischemic and non-ischemic HF may reveal new etiology-specific treatments.

A roadblock to gaining a more comprehensive molecular characterization of the different HF phenotypes has been the paucity of high quality human heart tissue with a well-defined clinical history. The cardiac transplant program described herein has enabled the development of an extensive repository of high quality diseased human heart tissues, each with a well-documented clinical history. In addition, hearts have been obtained from local organ procurement organizations that did not show signs of heart disease and were not used for transplantation. This heart repository was previously used for focused antibody-based proteomics screens, which showed that the abundance of the XIAP protein, a powerful inhibitor of apoptosis, diminishes in non-ischemic cardiomyopathy compared to non-failing control tissue.⁸

Interrogating the molecular biology of human heart tissue through antibody-based targeted approaches is limited to only proteins for which reagents are readily available, and data collection is slow and cumbersome. In contrast, unbiased LC/MS/MS based proteomics is an objective, high-throughput method which can be used to obtain a global assessment of the protein composition of a biological sample. In this investigation, cardiac tissue from the well-characterized human heart tissue bank was analyzed by LC/MS/MS proteomics, demonstrating for the first time, proteomic and phosphoproteomic changes in two types of clinically distinct end-stage human HF: the ischemic failing (IF) and the non-ischemic failing (NIF) heart. By utilizing a global proteomic approach with high resolution protein separation, disease-dependent alterations of the cardiac proteome in IF and NIF hearts were identified. The alterations in protein levels and residue-specific phosphorylation between the failing and non-failing groups may be pivotal to the understanding of HF progression.

### SUMMARY OF THE INVENTION

The present disclosure provides methods of assessing the risk of a subject for suffering ischemic or non-ischemic heart failure comprising, consisting of, or consisting essentially of quantifying the amount of at least one biomarker present in a biological sample derived from the subject, wherein the biomarker comprises, consists of, or consists essentially of a protein associated with ischemic (IF) or non-ischemic (NIF) heart failure.

One aspect of the present disclosure provides a method of determining the risk of, prognosis of, and/or diagnosis of ischemic or non-ischemic heart failure in a subject comprising, consisting of, or consisting essentially of quantifying the amount of at least one biomarker present in a biological sample derived from the subject, wherein the biomarker is associated with ischemic or non-ischemic heart failure.

Another aspect of the present disclosure provides a method of diagnosing ischemic or non-ischemic heart failure in a subject comprising, consisting of, or consisting essentially of: (a) obtaining a biological sample from a subject; (b) determining the expression level of one or more biomarkers that are associated with ischemic or non-ischemic heart failure in the biological sample; (c) comparing the expression level of the biomarkers in the biological sample with that of a control, wherein the presence of one or more of the biomarkers in the sample that is in an amount greater than that of the control indicates ischemic or non-ischemic heart failure; and (d) administering appropriate heart failure therapy if one or more of the biomarkers are expressed.

Another aspect of the present disclosure provides a method of determining the risk of a subject developing ischemic or non-ischemic heart failure comprising, consisting of, or consisting essentially of: (a) obtaining a biological sample from a subject; (b) determining the expression level of one or more biomarkers that are associated with ischemic or non-ischemic heart failure in the biological sample; (c) comparing the expression level of the biomarkers in the biological sample with that of a control, wherein the presence of one or more of the biomarkers in the sample that is in an amount greater than that of the control indicates ischemic or non-ischemic heart failure; and (d) administering an appropriate prophylactic heart failure therapy if one or more of the biomarkers are expressed.

Another aspect of the present disclosure provides a method of determining the prognosis of a subject developing, or having already developed, ischemic or non-ischemic heart failure comprising, consisting of, or consisting essentially of: (a) obtaining a biological sample from a subject; (b) determining the expression level of one or more biomarkers that are associated with ischemic or non-ischemic heart failure in the biological sample; (c) comparing the expression level of the biomarkers in the biological sample with that of a control, wherein the presence of one or more of the biomarkers in the sample that is in an amount greater than that of the control indicates ischemic or non-ischemic heart failure; and (d) administering an appropriate heart failure therapy or altering an already administered heart failure therapy, if one or more of the biomarkers are expressed.

Another aspect of the present disclosure provides a method of determining the efficacy of a heart failure treatment regime in a subject comprising, consisting of, or consisting essentially of: (a) determining a baseline value for the expression of one or more biomarkers associated with ischemic or non-ischemic heart failure; (b) administering to the subject a heart failure therapy regime; and (c) redetermining the expression levels of one or more biomarkers in the subject, wherein observed decreases in one or more of the biomarker expression levels is correlated with the efficacy of the therapeutic regimen.

Another aspect of the present disclosure provides a composition of matter comprising, consisting of, or consisting essentially of: (a) a probe array for determining an biomarker level in a sample, the array comprising of a plurality of probes that hybridizes to one or more biomarkers that are associated with ischemic or non-ischemic heart failure; or (b) a kit for determining a biomarker level in a sample, comprising the probe array of (a) and instructions for carrying out the determination of biomarker expression level in the sample. In certain embodiments the probe array of (a) further comprises a solid support with the plurality of probes attached thereto.

In some embodiments, the biomarker comprises a protein, a phosphoprotein, or combinations thereof.

In one embodiment, the biomarker(s) associated with ischemic heart failure comprise, consist of, or consist essentially of one or more of the following proteins: Ig alpha-2 chain C region, Carbonic anhydrase 1, Ig my chain C region, Hemoglobin subunit alpha, Ig alpha-1 chain C region, Hemoglobin subunit beta, Alpha-2-macroglobulin, serum amyloid A protein, and any combination thereof.

In another embodiment, the biomarker(s) associated with non-ischemic heart failure may comprise, consist of, or consist essentially of one or more of the following proteins: Carbonic anhydrase 3, Ig alpha-2 chain C region, Ig mu chain C region, Latent-transforming growth factor β-binding protein 2, Carbonic anhydrase 1, Ig alpha-1 chain C region, Hemoglobin subunit alpha, Asporin, Collagen alpha-3(VI) chain, Fibulin-2, Microfibril-associated glycoprotein 4, Hemoglobin subunit beta, Fibrinogen gamma chain, Ig lambda chain C regions, EGF-containing fibulin-like extracellular matrix protein 1, Fibulin-1, Ceruloplasmin, Dermatopontin, Ig gamma-2 chain C region, Coagulation factor IX, Myosin-2, ATP synthase subunit delta (mitochondrial), Serum amyloid A protein, and combinations thereof.

In another embodiment, the biomarker(s) associated with ischemic heart failure may comprise, consist of, or consist essentially of one or more of the following phosphopeptides: Alpha-2-HS-glycoprotein precursor (HTFMGVVSLGSPSGEVSHPR; SEQ ID NO:1); Alpha-2-HS-glycoprotein precursor (HTFMGVVSLGSPSGEVSHPR; SEQ ID NO:2); Myosin regulatory light chain MRLC2 (TFMGVVSLGSPSGEVSHPR; SEQ ID NO:3); Leiomodin-1 (GSPKPSPQPSPKPSPK; SEQ ID NO:4); Nexilin (EMLASDDEEDVSSKVEK; SEQ ID NO:5); Pyruvate dehydrogenase E1 component subunit α, somatic form, mitochondrial (YHGHSMSDPGVSYR; SEQ ID NO:6); Pyruvate dehydrogenase E1 component subunit α, somatic form, mitochondrial (YHGHSMSDPGVSYR; SEQ ID NO:7); Pyruvate dehydrogenase E1 component subunit α, somatic form, mitochondrial (YGMGTSVER; SEQ ID NO:8), and combinations thereof.

In another embodiment, the biomarker(s) associated with non-ischemic heart failure may comprise, consist of, or consist essentially of one or more of the following phosphopeptides: Alpha-2-HS-glycoprotein precursor (HTFMGVVSLGSPSGEVSHPR; SEQ ID NO:9); Heat shock protein HSP 90-beta (IEDVGSDEEDDSGK; SEQ ID NO:10); Sorbin and SH3 domain-containing protein 2 (SEPAVGPPR; SEQ ID NO:11); Sorbin and SH3 domain-containing protein 2 (DASSPVPPPHVPPPVPPLRPR; SEQ ID NO:12); Blood vessel epicardial substance (NSIASSSDSDDGLHQFLR; SEQ ID NO:13) and combinations thereof.

In some embodiments, the present invention provides a method of treating a cardiac disease or disorder and/or reducing fibrosis associated with a cardiac disease or disorder and/or improving cardiac function in a subject in need thereof, comprising administering to the subject an effective amount of a substance that modulates (e.g., down-regulates or upregulates) expression of nucleic acid that encodes fibulin 1, nucleic acid the encodes fibulin 2 and/or nucleic acid that encodes fibulin 3, in any combination, in cells of the subject, thereby treating the cardiac disease or disorder and/or reducing fibrosis associated with the cardiac disease or disorder and/or improving cardiac function in the subject.

Also provided herein is a method of treating a cardiac disease or disorder and/or reducing fibrosis associated with a cardiac disease or disorder and/or improving cardiac function in a subject in need thereof, comprising administering to the subject an effective amount of a substance that modulates (e.g., reduces or increases) the amount and/or activity of fibulin 1, fibulin 2 and/or fibulin 3, in any combination, thereby treating the cardiac disease or disorder and/or reducing fibrosis associated with the cardiac disease or disorder and/or improving cardiac function in the subject.

In addition, the present invention provides a method of treating a cardiac disease or disorder and/or improving cardiac function in a subject in need thereof, comprising administering to the subject an effective amount of a substance that modulates the expression of nucleic acid encoding fetuin A in cells of the subject, thereby treating the cardiac disease or disorder and/or reducing fibrosis associated with the cardiac disease or disorder and/or improving cardiac function in the subject.

Further provided herein is a method of treating a cardiac disease or disorder and/or improving cardiac function in a subject in need thereof, comprising administering to the subject an effective amount of a substance that modulates the amount and/or activity of fetuin A, thereby treating the cardiac disease or disorder and/or improving cardiac function in the subject.

In additional embodiments, the present invention provides a method of treating a cardiac disease or disorder and/or improving cardiac function in a subject in need thereof, comprising administering to the subject an effective amount of a substance that modulates CK2 activity, thereby treating the cardiac disease or disorder and/or improving cardiac function in the subject.

The present invention further provides a method of treating a cardiac disease or disorder and/or improving cardiac function in a subject in need thereof, comprising administering to the subject an effective amount of a substance that modulates the amount and/or activity of carbonic anhydrase 1, carbonic anhydrase 3 or both, thereby treating the cardiac disease or disorder and/or improving cardiac function in the subject.

Additionally provided herein is a method of treating a cardiac disease or disorder and/or improving cardiac function in a subject in need thereof, comprising administering to the subject an effective amount of a nucleic acid molecule that encodes the amino acid sequence of C6ORF142, wherein the amino acid sequence is modified to lack Ser67 phosphorylation, thereby treating the cardiac disease or disorder and/or improving cardiac function in the subject.

In some embodiments of this invention, a method is provided of treating a cardiac disease or disorder and/or improving cardiac function in a subject in need thereof, comprising administering to the subject an effective amount of a substance that modulates the amount and/or activity of C6ORF142 and/or inhibits phosphorylation of Ser67 of C6ORF142, thereby treating the cardiac disease or disorder and/or improving cardiac function in the subject.

Furthermore, the present invention provides a method of treating a cardiac disease or disorder and/or improving cardiac function in a subject in need thereof, comprising administering to the subject an effective amount of a substance that modulates expression of nucleic acid encoding Lyric and/or a substance that modulates the amount and/or activity of Lyric and/or modulates phosphorylation at Ser298, thereby treating the cardiac disease or disorder and/or improving cardiac function in the subject.

Also provided herein is a method of treating non-ischemic heart failure and/or improving cardiac function in a subject in need thereof, comprising administering to the subject an effective amount of a nucleic acid molecule that encodes the amino acid sequence of Lyric, wherein the amino acid sequence is modified to lack Ser298 phosphorylation, thereby treating the non-ischemic heart failure and/or improving cardiac function in the subject.

Further provided herein is a method of treating ischemic heart failure and/or improving cardiac function in a subject in need thereof, comprising administering to the subject an effective amount of a nucleic acid molecule that encodes the amino acid sequence of Lyric, wherein the amino acid sequence is modified to increase Ser298 phosphorylation, thereby treating the ischemic heart failure and/or improving cardiac function in the subject.

The present invention also provides a method of treating non-ischemic heart failure and/or improving cardiac function in a subject in need thereof, comprising administering to the subject an effective amount of a nucleic acid molecule that encodes the amino acid sequence of leiomodin-1, wherein the amino acid sequence is modified to lack Ser516 and/or Ser555 phosphorylation, thereby treating non ischemic heart failure and/or improving cardiac function in the subject.

In addition, the present invention provides a method of treating ischemic heart failure and/or improving cardiac function in a subject in need thereof, comprising administering to the subject an effective amount of a nucleic acid molecule that encodes an amino acid sequence of leiomodin-1, wherein the amino acid sequence is modified to increase Ser516 and/or Ser555 phosphorylation, thereby treating ischemic heart failure or improving cardiac function in the subject.

Further provided herein is a method of treating cardiac disease and/or improving cardiac function in a subject in need thereof, comprising administering to the subject an effective amount of a nucleic acid molecule that encodes an amino acid sequence of leiomodin-1, wherein the amino acid sequence is modified to increase Ser508, Ser512 and/or Ser520 phosphorylation, thereby treating the cardiac disease or disorder and/or improving cardiac function in the subject.

In additional embodiments, the present invention provides a method of treating a cardiac disease or disorder and/or improving cardiac function in a subject in need thereof, comprising administering to the subject an effective amount of a substance that modulates expression of nucleic acid encoding leiomodin-1 and/or a substance that modulates the amount and/or activity of leiomodin-1 and/or a substance that inhibits phosphorylation at Ser516 and/or Ser555, thereby treating the cardiac disease or disorder and/or improving cardiac function in the subject.

Furthermore, the present invention provides a method of treating a cardiac disease or disorder and/or improving cardiac function in a subject in need thereof, comprising administering to the subject an effective amount of a substance that upregulates the expression of nucleic acid encoding alpha 2 macroglobulin in cells of the subject, thereby treating the cardiac disease or disorder and/or improving cardiac function in the subject.

Also provided herein is a method of treating a cardiac disease or disorder and/or improving cardiac function in a subject in need thereof, comprising administering to the subject an effective amount of a substance that increases the amount of uncleaved alpha 2 macroglobulin and/or an effective amount of recombinant alpha 2 macroglobulin, thereby treating the cardiac disease or disorder and/or improving cardiac function in the subject.

Further provided in this invention is a method of treating a cardiac disease or disorder and/or improving cardiac function in a subject in need thereof, comprising administering to the subject an effective amount of a substance that modulates expression of nucleic acid encoding ceruloplasmin in cells of the subject, thereby treating the cardiac disease or disorder and/or improving cardiac function in the subject.

The present invention also includes a method of treating a cardiac disease or disorder and/or improving cardiac function and/or increasing cardiomyocyte survival in a subject in need thereof, comprising administering to the subject an effective amount of a substance that increases the amount of peptidyl prolyl cis trans isomerase, thereby treating the cardiac disease or disorder and/or improving cardiac function and/or increasing cardiomyocyte survival in the subject.

A method is also provided of treating a cardiac disease or disorder and/or improving cardiac function and/or protecting myocardium from further injury in a subject in need thereof, comprising administering to the subject an effective amount of a substance that increases the amount of Protein DJ1/PARK7, thereby treating the cardiac disease or disorder and/or improving cardiac function and/or protecting myocardium from further injury in the subject.

In some embodiments, the subject is a mammal. In other embodiments, the subject is a human.

In other embodiments, the biological sample is selected from the group consisting of tissues, cells, biopsies, blood, lymph, serum, plasma, urine, saliva, mucus, and tears. In certain embodiments, the sample comprises plasma.

Yet another aspect of the present disclosure provides for all that is disclosed and illustrated herein. The foregoing aspects and other features of the invention are explained in the following description, taken in connection with the accompanying drawings, as described herein below.

### BRIEF DESCRIPTION OF THE DRAWINGS

FIG. 1. Schematic showing the experimental approach according to one embodiment of the present disclosure. A. Illustration of the sample preparation and MS work flow. Left ventricular samples from 12 patients were homogenized by mechanical disruption in TRIZOL followed by an overnight digestion in trypsin. The sample was divided and 25 μg was spiked with 1.25 pmol ADH1_Yeast protein and subjected to label-free LC-MS/MS using a Synapt HDMS mass spectrometer to generate the unenriched label-free proteome. 600 μg of the trypsin digested sample was spiked with CASA1_BOVINE (30 fmol/μg lysate) and applied to a titanium oxide enrichment spin column. The eluant from the column was spiked with ADH1_YEAST and subjected to label-free LC-MS/MS using an Orbitrap XL mass spectrometer. B. Reproducibility/Internal Control. Relative levels of the ADH1 protein spiked into each heart lysate as determined by chromatographic peak intensity. C. Venn Diagram demonstrating the protein and phosphoprotein yield and overlap.

FIG. 2. Principal Components Analysis and Hierarchical Clustering. The expression data for all peptides from the unenriched (A) and phosphopeptide enriched (B) samples were used to perform Principal Components Analysis (PCA) after z-score transformation of the peptide intensities. The top two principal components are plotted in each figure, showing no extreme outlier samples among the twelve individual patients tested, either among the unenriched samples or phosphopeptide-enriched. The statistically-significant differentially expressed peptides for each experiment were used to calculate 2D hierarchical clusters in order to view sample-to-sample relationships within these differentially expressed signals, at the unenriched proteome level from Table 2 (C) and from the phosphoproteome in Table 4 (D).

FIG. 3 is a graph showing the abundance and differential profile of proteins from IF and NIF human hearts. A. Abundances of proteins from IF and NIF human hearts. Each dot represents a protein whose abundance was >2 fold changed compared to NF control human hearts (ANOVA p<0.05). IF proteins are marked with ⋄ and NIF proteins are marked with ▪. The X axis represents the fold change while the Y axis is the log base 10 of the p values. Dashed Horizontal lines represent an absolute value fold change of 2. B. Differential Phosphopeptide profile of proteins from IF and NIF human hearts. Each dot represents an individual phosphopeptide whose abundance was >6 fold change compared to NF control human hearts. IF is depicted with O and NIF with ▪. Only those peptides whose fold change was statistically significant as determined by ANOVA with an 80% statistical power are shown. Vertical Dashed Line denotes fold change of absolute value of 6. *, †, ‡ represent three different phosphopeptide sites on the ODPA moiety of the pyruvate dehydrogenase protein complex which is also represented in Table 9. §, ∥, indicates different phosphopeptide sites on FETUA and are reflected on Table 3A and 3B. # and ** indicates different phosphopeptide sites on the SRBS2 protein (in NIF) and corresponds to Table 3A and 3B as well.

FIG. 4 shows Western blot validations of selected statistically significant proteins identified by mass spectrometry proteomics. A. Protein extracts from IF, NIF, or NF samples (40 μg) were subjected to polyacrylamide gel electrophoresis. Replica nitrocellulose blots were incubated with anti-carbonic anhydrase, -ceruloplasmin, -fibulin 1, -fibulin 2, -serum amyloid A, -fetuin A, -alpha 2 macroglobulin, or -sacromeric actin. Visualization of the blots is shown. B. Quantitation of carbonic anhydrase 1, ceruloplasmin, fibulin 1, fibulin 2, serum amyloid A, fetuin A, and total protein and cleavage products of alpha 2 macroglobulin as determined by densitometry analysis and expressed as relative intensity (arbitrary units (AU)) normalized to actin levels. *: significantly elevated relative to non-failing, †: significantly elevated relative to both NIF and IF.

FIG. 5 is a diagram showing the interaction network of non-ischemic failing hearts. Relevant interactions of the differentially expressed proteins and their relationships with certain disease/pathologies are depicted.

FIG. 6 shows Western blot analyses of central proteins identified from pathway analysis. A. Protein extracts from IF, NIF, or NF samples (40 μg) were subjected to polyacrylamide gel electrophoresis. Replica nitrocellulose blots were incubated with anti-SMAD3, -AKT, -MMP14, -AHR or -sarcomeric actin. Visualization of the blots is shown. B. Quantitation of SMAD3, AKT, MMP14, and AHR as determined by densitometry analysis and expressed as relative intensity (arbitrary units (AU)) normalized to actin levels. *: significantly elevated relative to non-failing, †: significantly elevated relative to both NIF and IF.

FIG. 7. Casein kinase phosphorylation targets. Nine of the differentially phosphorylated proteins shown in the tables herein are possible targets of casein kinase.

FIG. 8. Evaluation of metabolic story surrounding PDH. PDH, pyruvate dehydrogenase; LDH, lactate dehydrogenase; NF, non-failing; IF, ischemic failing; NIF, non-ischemic failing; RFU, relative fluorescent unit. No statistical differences were observed.

### DETAILED DESCRIPTION OF THE INVENTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount (e.g., an amount of methylation) and the like, is meant to include variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP §2111.03. Thus, the term “consisting essentially of” when used in a claim of this invention is not intended to be interpreted to be equivalent to “comprising.”

Unless otherwise defined, all technical terms used herein have the same meaning as commonly understood by one of ordinary skill in the art to which this disclosure belongs.

### DEFINITIONS

As used herein, the term “biomarker” refers to a naturally occurring biological molecule present in a subject at varying concentrations useful in predicting the risk or incidence of a disease or a condition, such as ischemic and/or non-ischemic heart failure. For example, the biomarker can be a protein present in higher or lower amounts in a subject at risk for ischemic and/or non-ischemic heart failure. The biomarker can include proteins, phosphopeptides, nucleic acids, ribonucleic acids, etc. and combinations thereof used as an indicator or marker for ischemic and/or non-ischemic heart failure in a subject. In some embodiments, the biomarker comprises a protein. In other embodiments, the biomarker comprises a phosphopeptide. It is also within the scope of the present disclosure that a panel of biomarkers for ischemic and/or non-ischemic heart failure may comprise only proteins, only phosphopeptides, or a combination of both proteins and phosphopeptides.

In one embodiment, the biomarker(s) associated with ischemic heart failure comprise, consist of, or consist essentially of one or more of the following proteins: Ig alpha-2 chain C region, Carbonic anhydrase 1, Ig my chain C region, Hemoglobin subunit alpha, Ig alpha-1 chain C region, Hemoglobin subunit beta, Alpha-2-macroglobulin, serum amyloid A protein, and combinations thereof.

In another embodiment, the biomarker(s) associated with non-ischemic heart failure may comprise, consist of, or consist essentially of one or more of the following proteins: Carbonic anhydrase 3, Ig alpha-2 chain C region, Ig mu chain C region, Latent-transforming growth factor β-binding protein 2, Carbonic anhydrase 1, Ig alpha-1 chain C region, Hemoglobin subunit alpha, Asporin, Collagen alpha-3(VI) chain, Fibulin-2, Microfibril-associated glycoprotein 4, Hemoglobin subunit beta, Fibrinogen gamma chain, Ig lambda chain C regions, EGF-containing fibulin-like extracellular matrix protein 1, Fibulin-1, Ceruloplasmin, Dermatopontin, Ig gamma-2 chain C region, Coagulation factor IX, Myosin-2, ATP synthase subunit delta (mitochondrial), Serum amyloid A protein, and combinations thereof.

In another embodiment, the biomarker(s) associated with ischemic heart failure may comprise, consist of, or consist essentially of one or more of the following phosphopeptides: Alpha-2-HS-glycoprotein precursor (HTFMGVVSLGSPSGEVSHPR; SEQ ID NO:1); Alpha-2-HS-glycoprotein precursor (HTFMGVVSLGSPSGEVSHPR; SEQ ID NO:2); Myosin regulatory light chain MRLC2 (TFMGVVSLGSPSGEVSHPR; SEQ ID NO:3); Leiomodin-1 (GSPKPSPQPSPKPSPK; SEQ ID NO:4); Nexilin (EMLASDDEEDVSSKVEK; SEQ ID NO:5); Pyruvate dehydrogenase E1 component subunit α, somatic form, mitochondrial (YHGHSMSDPGVSYR; SEQ ID NO:6); Pyruvate dehydrogenase E1 component subunit α, somatic form, mitochondrial (YHGHSMSDPGVSYR; SEQ ID NO:7); Pyruvate dehydrogenase E1 component subunit α, somatic form, mitochondrial (YGMGTSVER; SEQ ID NO:8), and combinations thereof.

In another embodiment, the biomarker(s) associated with non-ischemic heart failure may comprise, consist of, or consist essentially of one or more of the following phosphopeptides: Alpha-2-HS-glycoprotein precursor (HTFMGVVSLGSPSGEVSHPR; SEQ ID NO:9); Heat shock protein HSP 90-beta (IEDVGSDEEDDSGK; SEQ ID NO:10); Sorbin and SH3 domain-containing protein 2 (SEPAVGPPR; SEQ ID NO:11); Sorbin and SH3 domain-containing protein 2 (DASSPVPPPHVPPPVPPLRPR; SEQ ID NO:12); Blood vessel epicardial substance (NSIASSSDSDDGLHQFLR; SEQ ID NO:13) and combinations thereof.

As used herein, the term “ischemic heart failure (IF)” and “myocardial ischemia” are used interchangeably and refer to those diseases/conditions of the heart characterized by significantly impaired left ventricular function that results from ischemia (i.e., reduced blood supply) of the heart muscle, usually due to coronary artery disease (e.g., atherosclerosis). The term “non-ischemic heart failure (NIF)” refers to those diseases/conditions of the heart that are not related to coronary artery disease, and include dilated cardiomyopathy, hypertrophic cardiomyopathy, restrictive cardiomyopathy, and arrhythmogenic right ventricular dysplasia (ARVD). Non-ischemic heart failure has a range of etiologies, including congenital, infectious agents, autoimmune, and idiopathic causes.

“About” is used to provide flexibility to a numerical range endpoint by providing that a given value may be “slightly above” or “slightly below” (e.g., by 0.5%, 1%, 2%, 3%, 5%, 10%, etc.) the endpoint without affecting the desired result.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. For example, such therapies may include antihypertensive therapy, anticholesterol therapy, heart transplantation, palliation with continuous intravenous inotropic support, installation of a pacemaker, cardiac resynchronization therapy (CRT), and the like. Such treatments are well known and particular to the patient and can be readily determined by one skilled in the art

The term “effective amount” or “therapeutically effective amount” refers to an amount sufficient to effect beneficial or desirable biological and/or clinical results.

The present disclosure provides biomarkers useful for determining the risk of ischemic and non-ischemic heart failure in a subject. The present disclosure also provides methods of using such biomarker expression profiles to monitor a subject's response to treatment (e.g., efficacy of a treatment or therapy regimen) for conditions such as ischemic and non-ischemic heart failure.

Advantageously, the methods of the present disclosure are noninvasive, highly specific, and sensitive.

In one embodiment, the present disclosure profiles biomarkers found in the plasma for the diagnosis and prognosis of ischemic and/or non-ischemic heart failure.

In one embodiment, the present disclosure identifies plasma protein profiles as biomarkers for determining the risk of, prognosis of, and/or diagnosis of conditions such as ischemic and/or non-ischemic heart failure. The inventors have determined that certain biomarkers are directly involved in ischemic and/or non-ischemic heart failure, and their expression pattern in plasma can be associated with the pathophysiological status of ischemic and/or non-ischemic heart failure. It was discovered that these biomarker expression patterns in subjects at risk of ischemic and/or non-ischemic heart failure are distinctly different from that of normal controls (non-failing [NF]).

### Biomarkers for Ischemic and Non-Ischemic Heart Failure

One aspect of the present disclosure provides biomarkers useful for determining the risk of, prognosis of, and/or diagnosis of conditions such as ischemic and/or non-ischemic heart failure. In one embodiment, the present disclosure provides biomarkers that are differentially expressed, such as upregulated, down-regulated, or disregulated in a condition such as ischemic and/or non-ischemic heart failure, as compared to normal populations who do not have the condition, such as ischemic and/or non-ischemic heart failure.

In some embodiments, the biomarker comprises a protein. In other embodiments, the biomarker comprises a phosphopeptide. In one embodiment, the biomarker(s) associated with ischemic heart failure comprise, consist of, or consist essentially of one or more of the following proteins: Ig alpha-2 chain C region, Carbonic anhydrase 1, Ig my chain C region, Hemoglobin subunit alpha, Ig alpha-1 chain C region, Hemoglobin subunit beta, Alpha-2-macroglobulin, serum amyloid A protein, and combinations thereof.

In another embodiment, the biomarker(s) associated with non-ischemic heart failure may comprise, consist of, or consist essentially of one or more of the following proteins: Carbonic anhydrase 3, Ig alpha-2 chain C region, Ig mu chain C region, Latent-transforming growth factor β-binding protein 2, Carbonic anhydrase 1, Ig alpha-1 chain C region, Hemoglobin subunit alpha, Asporin, Collagen alpha-3(VI) chain, Fibulin-2, Microfibril-associated glycoprotein 4, Hemoglobin subunit beta, Fibrinogen gamma chain, Ig lambda chain C regions, EGF-containing fibulin-like extracellular matrix protein 1, Fibulin-1, Ceruloplasmin, Dermatopontin, Ig gamma-2 chain C region, Coagulation factor IX, Myosin-2, ATP synthase subunit delta (mitochondrial), Serum amyloid A protein, C6ORF142, and combinations thereof.

C6ORF142 (GenBank Accession No. EAX04436.1)

| | |
|---|---|
| 1 | MELEKREKRS LLNKNLEEKL TVSAGGSEAK PLIFTFVPTV |
| | RRLPTHTQLA DTSKFLVKIP |
| 61 | EESSDKSPET VNRSKSNDYL TLNAGSQQER DQAKLTCPSE |
| | VSGTILQERE FEANKLQGMQ |
| 121 | QSDLFKAEYV LIVDSEGEDE AASRKVEQGP PGGIGTAAVR |
| | PKSLAISSSL VSDVVRPKTQ |
| 181 | GTDLKTSSHP EMLHGMAPQQ KHGQQYKTKS SYKAFAAIPT |
| | NTLLLEQKAL DEPAKTESVS |
| 241 | KDNTLEPPVE LYFPAQLRQQ TEELCATIDK VLQDSLSMHS |
| | SDSPSRSPKT LLGSDTVKTP |
| 301 | TTLPRAAGRE TKYANLSSPT STVSESQLTK PGVIRPVPVK |
| | SRILLKKEEE VYEPNPFSKY |
| 361 | LEDNSDLFSE QDVTVPPKPV SLHPLYQTKL YPPAKSLLHP |
| | QTLSHADCLA PGPFSHLSFS |
| 421 | LSDEQENSHT LLSHNACNKL SHPMVAIPEH EALDSKEQ |

In another embodiment, the biomarker(s) associated with ischemic heart failure may comprise, consist of, or consist essentially of one or more of the following phosphopeptides: Alpha-2-HS-glycoprotein precursor (HTFMGVVSLGSPSGEVSHPR); Alpha-2-HS-glycoprotein precursor (HTFMGVVSLGSPSGEVSHPR); Myosin regulatory light chain MRLC2 (TFMGVVSLGSPSGEVSHPR); Leiomodin-1 (GSPKPSPQPSPKPSPK); Nexilin (EMLASDDEEDVSSKVEK); Pyruvate dehydrogenase E1 component subunit α, somatic form, mitochondrial (YHGHSMSDPGVSYR); Pyruvate dehydrogenase E1 component subunit α, somatic form, mitochondrial (YHGHSMSDPGVSYR); Pyruvate dehydrogenase E1 component subunit α, somatic form, mitochondrial (YGMGTSVER), and combinations thereof.

In another embodiment, the biomarker(s) associated with non-ischemic heart failure may comprise, consist of, or consist essentially of one or more of the following phosphopeptides: Alpha-2-HS-glycoprotein precursor (HTFMGVVSLGSPSGEVSHPR); Heat shock protein HSP 90-beta (IEDVGSDEEDDSGK); Sorbin and SH3 domain-containing protein 2 (SEPAVGPPR); Sorbin and SH3 domain-containing protein 2 (DASSPVPPPHVPPPVPPLRPR); Blood vessel epicardial substance (NSIASSSDSDDGLHQFLR) and combinations thereof.

In some embodiments, the biomarkers are selected from one or more biomarkers provided in Tables 2-4 that are up-regulated or over-expressed in a subject at risk for ischemic and/or non-ischemic heart failure. In some embodiments, the up-regulation or over-expression of one or more of the biomarkers in the subject's biological sample, when compared to a control, indicates that the subject is at risk of ischemic and/or non-ischemic heart failure.

In some specific embodiments, the biomarkers are selected from one or more biomarkers up-regulated or over-expressed more than 50-fold, 40-fold, 30-fold, 20-fold, 15-fold, 10-fold, 9-fold, 8-fold, 7-fold, 6-fold, 5-fold, 4-fold, 3-fold, 2-fold, or 1-fold in a subject at risk of ischemic and/or non-ischemic heart failure, when compared to a control. In some embodiments, the up-regulation or over-expression of the biomarker in the subject's biological sample, when compared to a control, indicates that the subject is at risk of ischemic and/or non-ischemic heart failure.

### Methods Using Biomarkers

In one embodiment, the present disclosure provides a method for assessing the risk of ischemic and/or non-ischemic heart failure in a subject comprising, consisting of, or consisting essentially of:

(a) determining a biomarker expression profile (expression level) in a biological sample from the subject;
(b) characterizing the subject's biomarker profile; and
(c) comparing the subject's biomarker profile with the biomarker profile of a control from subjects not at risk of ischemic and/or non-ischemic heart failure; and
(d) administering an appropriate heart failure therapy if one or more of the biomarkers are expressed.

In another embodiment, the present disclosure provides a method for determining the risk of a subject developing a condition such as ischemic and/or non-ischemic heart failure comprising, consisting of, or consisting essentially of:

(a) determining a biomarker expression profile (expression level) in a biological sample from the subject;
(b) characterizing the subject's biomarker profile; and
(c) comparing the subject's biomarker profile with the biomarker profile of a control profile from subjects not at risk of ischemic and/or non-ischemic heart failure; and
(d) administering an appropriate prophylactic heart failure therapy if one or more of the biomarkers are expressed.

In yet another embodiment, the present disclosure provides a method for determining the prognosis of a subject developing, or having already developed, a condition such as ischemic and/or non-ischemic heart failure comprising, consisting of, or consisting essentially of:

(a) determining a biomarker expression profile (expression level) in a biological sample from the subject;
(b) characterizing the subject's biomarker profile; and
(c) comparing the subject's biomarker profile with the biomarker profile of a control profile from subjects not at risk of ischemic and/or non-ischemic heart failure; and
(d) administering appropriate heart failure therapy or altering an already existing heart failure therapy if one or more of the biomarkers are expressed.

In one embodiment, the method further includes obtaining the biological sample from the subject. In one embodiment, the diagnosis and/or prognosis of a condition such as ischemic and/or non-ischemic heart failure can be determined by comparing the subjects biomarker profile to a reference biomarker profile, such as one that corresponds to biological samples obtained from a normal population that do not have a condition such as ischemic and/or non-ischemic heart failure (e.g., non-failing [NF]), or that corresponds to biological samples obtained from a population that have a condition such as ischemic and/or non-ischemic heart failure. Optionally, the reference profile comprises multiple biomarker expression profiles, with each corresponding to a different stage of a condition such as ischemic and/or non-ischemic heart failure.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. Preferably, the subject is a human patient that is at for, or suffering from, ischemic and/or non-ischemic heart failure.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. In one embodiment, the biological sample is a blood sample (such as a plasma sample). A biological sample may be obtained directly from a subject (e.g., by blood or tissue sampling) or from a third party (e.g., received from an intermediary, such as a healthcare provider or lab technician).

In some embodiments, the present disclosure provides methods for diagnosing a condition such as ischemic and/or non-ischemic heart failure by characterizing one or more of the biomarkers provided in Tables 2-4. In some embodiments, the present disclosure provides methods for diagnosing conditions such as ischemic and/or non-ischemic heart failure by characterizing one or more of the biomarkers provided in Tables 2-4, wherein the up-regulation or over-expression of the biomarker, when compared to a control, indicates that the subject has a condition such as ischemic and/or non-ischemic heart failure.

In other embodiments, the present disclosure provides methods for determining the risk of a subject developing a condition such as ischemic and/or non-ischemic heart failure by characterizing one or more of the biomarkers provided in Tables 2-4. In some embodiments, the present disclosure provides methods for determining the risk of a subject developing a condition such as ischemic and/or non-ischemic heart failure by characterizing one or more of the biomarkers provided herein, wherein the up-regulation or over-expression of the biomarker, when compared to a control, indicates that the subject has a condition such as ischemic and/or non-ischemic heart failure.

In yet other embodiments, the present disclosure provides methods for determining the prognosis of a subject having a condition such as ischemic and/or non-ischemic heart failure by characterizing one or more of the biomarkers provided in Tables 2-4. In some embodiments, the present disclosure provides methods for determining the prognosis of a subject having a condition such as ischemic and/or non-ischemic heart failure by characterizing one or more of the biomarkers provided in Tables 2-4, wherein the up-regulation or over-expression of the biomarker, when compared to a control, indicates that the subject has a condition such as ischemic and/or non-ischemic heart failure.

### Methods of Determining Efficacy of Treatment Using Biomarkers

Another aspect of the present disclosure provides for methods for monitoring the treatment of conditions such as ischemic and/or non-ischemic heart failure. In one embodiment, the method comprises a method of determining the efficacy of a heart failure treatment regime (e.g., antihypertensive therapy) in a subject comprising, consisting of, or consisting essentially of: (a) determining a baseline value for the expression of one or more biomarkers associated with ischemic and/or non-ischemic heart failure; (b) administering to the subject an ischemic and/or non-ischemic heart failure therapy regime; and (c) redetermining the expression levels of one or more biomarkers in the subject, wherein observed decreases in one or more or the biomarker expression levels is correlated with the efficacy of the therapeutic regimen. In instances where a decrease in the biomarker expression is not seen, a change in treatment may be warranted. Such a determination, and the different type of treatment to employ, can be made readily determined by one skilled in the art.

In a further embodiment, the present invention provides a method of determining the efficacy of a treatment regimen for a cardiac disease or disorder in a subject in need thereof, comprising: a) measuring an amount of a biomarker of this invention that is associated with a cardiac disease or disorder (e.g., increased/decreased; phosphorylated/not phosphorylated and/or cleaved/uncleaved) in a sample obtained from a subject for whom a treatment regimen for the cardiac disease or disorder is indicated prior to administration of the treatment regimen; b) administering the treatment regimen to the subject; c) measuring the amount of the biomarker in a sample obtained from the subject after administration of the treatment regimen to the subject; and d) comparing the amount of the biomarker measured in (a) with the amount of the biomarker measured in (c), wherein a decrease or increase; change in phosphorylation and/or change in cleavage product and/or pattern of the biomarker measured in (c) identifies the treatment regimen as an effective treatment regimen. Any of the proteins recited in the tables herein can be a biomarker of this invention.

As one nonlimiting example the biomarker can be cleavage products of alpha 2 macroglobulin and the amount of cleavage products is what is measured before and after the treatment regimen is administered and a decrease in cleavage products after treatment identifies the treatment regimen as effective.

As another nonlimiting example, the biomarker can be galactin 3 binding protein (GBP) and the amount of GBP is what is measured before and after the treatment regimen is administered and a change in the amount of GBP (e.g., increased or decreased) identifies the treatment regimen as effective or not.

Nonlimiting examples of a treatment regimen to treat a cardiac disease or disorder of this invention include a left, right or bi-ventricular assist device (LVAD, RVAD, biVAD), an oral medication, a para-aortic balloon pump, preemptive medication in earlier stages of heart failure to prevent/slow progression, as are known in the art.

As used herein, a “cardiac disease or disorder” includes but is not limited to cardiovascular disease, heart disease, heart injury, cardiomyopathy, non-ischemic heart failure, ischemic heart failure, and the like as would be known in the art.

The present invention also provides a method of determining when a treatment regimen for a cardiac disease or disorder will be effective in a subject for whom such a treatment regimen is indicated, comprising monitoring a biomarker of this invention (e.g., by detecting a change in amount, a change in phosphorylation, a change in cleavage product amount and/or pattern) and administering the treatment regimen when the biomarker reaches a predetermined threshold amount or level or percentage of the biomarker that identifies the subject as having a cardiac disease or disorder status for which the treatment regimen would be effective. Any of the proteins recited in the tables herein can be a biomarker that can be employed in these methods.

### Compositions

Another aspect of the present disclosure provides a composition comprising, consisting of, or consisting essentially of: (a) a probe array for determining a biomarker level in a sample, the array comprising of a plurality of probes that hybridizes to one or more biomarkers that are associated with ischemic and/or non-ischemic heart failure; or (b) a kit for determining a biomarker level in a sample, comprising the probe array of (a) and instructions for carrying out the determination of biomarker expression level in the sample. In certain embodiments the probe array of (a) further comprises a solid support with the plurality of probes attached thereto. In some embodiments, the present invention provides a panel and/or a kit comprising two or more, in any combination, of: a) an antibody that specifically binds 60S ribosomal protein; b) an antibody that specifically binds myosin regulatory light polypeptide 9; c) an antibody that specifically binds putative annexin A2; and d) ANKRD26 like family C member.

### Therapeutic Methods

The present invention provides various therapeutic methods. Any of the methods described herein can be employed in connection with identification, detection and/or monitoring of changes in any of the biomarkers of this invention.

In some embodiments, the present invention provides a method of treating a cardiac disease or disorder and/or reducing fibrosis associated with a cardiac disease or disorder and/or improving cardiac function in a subject in need thereof, comprising administering to the subject an effective amount of a substance that modulates (e.g., down-regulates or upregulates) expression of nucleic acid that encodes fibulin 1, nucleic acid the encodes fibulin 2 and/or nucleic acid that encodes fibulin 3, in any combination, in cells of the subject, thereby treating the cardiac disease or disorder and/or reducing fibrosis associated with the cardiac disease or disorder and/or improving cardiac function in the subject.

Also provided herein is a method of treating a cardiac disease or disorder and/or reducing fibrosis associated with a cardiac disease or disorder and/or improving cardiac function in a subject in need thereof, comprising administering to the subject an effective amount of a substance that modulates (e.g., reduces or increases) the amount and/or activity of fibulin 1, fibulin 2 and/or fibulin 3, in any combination, thereby treating the cardiac disease or disorder and/or reducing fibrosis associated with the cardiac disease or disorder and/or improving cardiac function in the subject.

In addition, the present invention provides a method of treating a cardiac disease or disorder and/or improving cardiac function in a subject in need thereof, comprising administering to the subject an effective amount of a substance that modulates the expression of nucleic acid encoding fetuin A in cells of the subject, thereby treating the cardiac disease or disorder and/or reducing fibrosis associated with the cardiac disease or disorder and/or improving cardiac function in the subject.

Further provided herein is a method of treating a cardiac disease or disorder and/or improving cardiac function in a subject in need thereof, comprising administering to the subject an effective amount of a substance that modulates the amount and/or activity of fetuin A, thereby treating the cardiac disease or disorder and/or improving cardiac function in the subject.

In additional embodiments, the present invention provides a method of treating a cardiac disease or disorder and/or improving cardiac function in a subject in need thereof, comprising administering to the subject an effective amount of a substance that modulates CK2 activity, thereby treating the cardiac disease or disorder and/or improving cardiac function in the subject.

The present invention further provides a method of treating a cardiac disease or disorder and/or improving cardiac function in a subject in need thereof, comprising administering to the subject an effective amount of a substance that modulates the amount and/or activity of carbonic anhydrase 1, carbonic anhydrase 3 or both, thereby treating the cardiac disease or disorder and/or improving cardiac function in the subject.

Additionally provided herein is a method of treating a cardiac disease or disorder and/or improving cardiac function in a subject in need thereof, comprising administering to the subject an effective amount of a nucleic acid molecule that encodes the amino acid sequence of C6ORF142, wherein the amino acid sequence is modified to lack Ser67 phosphorylation, thereby treating the cardiac disease or disorder and/or improving cardiac function in the subject.

In some embodiments of this invention, a method is provided of treating a cardiac disease or disorder and/or improving cardiac function in a subject in need thereof, comprising administering to the subject an effective amount of a substance that modulates the amount and/or activity of C6ORF142 and/or inhibits phosphorylation of Ser67 of C6ORF142, thereby treating the cardiac disease or disorder and/or improving cardiac function in the subject.

Furthermore, the present invention provides a method of treating a cardiac disease or disorder and/or improving cardiac function in a subject in need thereof, comprising administering to the subject an effective amount of a substance that modulates expression of nucleic acid encoding Lyric and/or a substance that modulates the amount and/or activity of Lyric and/or modulates phosphorylation at Ser298, thereby treating the cardiac disease or disorder and/or improving cardiac function in the subject.

Also provided herein is a method of treating non-ischemic heart failure and/or improving cardiac function in a subject in need thereof, comprising administering to the subject an effective amount of a nucleic acid molecule that encodes the amino acid sequence of Lyric, wherein the amino acid sequence is modified to lack Ser298 phosphorylation, thereby treating the non-ischemic heart failure and/or improving cardiac function in the subject.

Further provided herein is a method of treating ischemic heart failure and/or improving cardiac function in a subject in need thereof, comprising administering to the subject an effective amount of a nucleic acid molecule that encodes the amino acid sequence of Lyric, wherein the amino acid sequence is modified to increase Ser298 phosphorylation, thereby treating the ischemic heart failure and/or improving cardiac function in the subject.

The present invention also provides a method of treating non-ischemic heart failure and/or improving cardiac function in a subject in need thereof, comprising administering to the subject an effective amount of a nucleic acid molecule that encodes the amino acid sequence of leiomodin-1, wherein the amino acid sequence is modified to lack Ser516 and/or Ser555 phosphorylation, thereby treating non ischemic heart failure and/or improving cardiac function in the subject.

In addition, the present invention provides a method of treating ischemic heart failure and/or improving cardiac function in a subject in need thereof, comprising administering to the subject an effective amount of a nucleic acid molecule that encodes an amino acid sequence of leiomodin-1, wherein the amino acid sequence is modified to increase Ser516 and/or Ser555 phosphorylation, thereby treating ischemic heart failure or improving cardiac function in the subject.

Further provided herein is a method of treating cardiac disease and/or improving cardiac function in a subject in need thereof, comprising administering to the subject an effective amount of a nucleic acid molecule that encodes an amino acid sequence of leiomodin-1, wherein the amino acid sequence is modified to increase Ser508, Ser512 and/or Ser520 phosphorylation, thereby treating the cardiac disease or disorder and/or improving cardiac function in the subject.

In additional embodiments, the present invention provides a method of treating a cardiac disease or disorder and/or improving cardiac function in a subject in need thereof, comprising administering to the subject an effective amount of a substance that modulates expression of nucleic acid encoding leiomodin-1 and/or a substance that modulates the amount and/or activity of leiomodin-1 and/or a substance that inhibits phosphorylation at Ser516 and/or Ser555, thereby treating the cardiac disease or disorder and/or improving cardiac function in the subject.

Furthermore, the present invention provides a method of treating a cardiac disease or disorder and/or improving cardiac function in a subject in need thereof, comprising administering to the subject an effective amount of a substance that upregulates the expression of nucleic acid encoding alpha 2 macroglobulin in cells of the subject, thereby treating the cardiac disease or disorder and/or improving cardiac function in the subject.

Also provided herein is a method of treating a cardiac disease or disorder and/or improving cardiac function in a subject in need thereof, comprising administering to the subject an effective amount of a substance that increases the amount of uncleaved alpha 2 macroglobulin and/or an effective amount of recombinant alpha 2 macroglobulin, thereby treating the cardiac disease or disorder and/or improving cardiac function in the subject.

Further provided in this invention is a method of treating a cardiac disease or disorder and/or improving cardiac function in a subject in need thereof, comprising administering to the subject an effective amount of a substance that modulates expression of nucleic acid encoding ceruloplasmin in cells of the subject, thereby treating the cardiac disease or disorder and/or improving cardiac function in the subject.

The present invention also includes a method of treating a cardiac disease or disorder and/or improving cardiac function and/or increasing cardiomyocyte survival in a subject in need thereof, comprising administering to the subject an effective amount of a substance that increases the amount of peptidyl prolyl cis trans isomerase, thereby treating the cardiac disease or disorder and/or improving cardiac function and/or increasing cardiomyocyte survival in the subject.

A method is also provided of treating a cardiac disease or disorder and/or improving cardiac function and/or protecting myocardium from further injury in a subject in need thereof, comprising administering to the subject an effective amount of a substance that increases the amount of Protein DJ1/PARK7, thereby treating the cardiac disease or disorder and/or improving cardiac function and/or protecting myocardium from further injury in the subject.

As used herein, “modulate” or “modulating” means to change or alter in either direction; e.g., upregulate or downregulate; decrease or increase; enhance or diminish, etc.

In some of the methods of this invention, an amino acid sequence can be modified to lack the ability to be phosphorylated at a particular site by replacing an amino acid that can be phosphorylated (e.g., Ser, Thr, Tyr) with an amino acid that cannot be phosphorylated. In some methods of this invention, an amino acid sequence can be modified to increase phosphorylation at a particular site by altering the consensus sequence of the amino acid to make the site a stronger target for phosphorylation.

### Sample

The present disclosure provides a method of determining the risk of, prognosis of, and/or diagnosis of a condition such as ischemic and/or non-ischemic heart failure on at least one sample obtained from an individual. The individual may be any mammal, but is preferably a human.

The present disclosure may involve obtaining more than one sample, such as two samples, such as three samples, four samples or more from individuals, and preferably the same individual. This allows the relative comparison of expression both as in the presence or absence of at least one protein and/or phosphopeptide and/or the level of expression of the at least one protein and/or phosphopeptide between the two samples. Alternatively, a single sample may be compared against a “standardized” sample, such a sample comprising material or data from several samples, preferably also from several individuals.

### Sample Preparation

Before analyzing the sample, it will often be desirable to perform one or more sample preparation operations upon the sample. Typically, these sample preparation operations will include such manipulations as concentration, suspension, extraction of intracellular material, e.g., proteins/phosphopeptides from tissue/whole cell samples and the like.

Any method required for the processing of a sample prior to detection by any of the methods noted herein falls within the scope of the present disclosure. These methods are typically well known by a person skilled in the art.

### Detection

It is within the general scope of the present disclosure to provide methods for the detection of protein biomarker. An aspect of the present disclosure relates to the detection of the proteins as described in the plots and graphs of the figures contained herein. As used herein, the term “detect” or “determine the presence of” refers to the qualitative measurement of undetectable, low, normal, or high concentrations of one or more biomarkers such as, for example, nucleic acids, ribonucleic acids, or polypeptides, proteins, phosphopeptides and other biological molecules. Detection may include 1) detection in the sense of presence versus absence of one or more biomarkers as well as 2) the registration/quantification of the level or degree of expression of one or more biomarkers, depending on the method of detection employed. The term “quantify” or “quantification” may be used interchangeable, and refer to a process of determining the quantity or abundance of a substance in a sample (e., a biomarker), whether relative or absolute. For example, quantification may be determined by methods including but not limited to, micro-array analysis, qRT-PCR, band intensity on a Northern or Western blot, or by various other methods known in the art.

The detection of one or more biomarker molecules allows for the classification, diagnosis and prognosis of a condition such as ischemic and/or non-ischemic heart failure. The classification of such conditions is of relevance both medically and scientifically and may provide important information useful for the diagnosis, prognosis and treatment of the condition. The diagnosis of a condition such as ischemic and/or non-ischemic heart failure is the affirmation of the presence of the condition, as is the object of the present disclosure, on the expression of at least one biomarker herein. Prognosis is the estimate or prediction of the probable outcome of a condition such as ischemic and/or non-ischemic heart failure and the prognosis of such is greatly facilitated by increasing the amount of information on the particular condition. The method of detection is thus a central aspect of the present disclosure.

Any method of detection falls within the general scope of the present disclosure. The detection methods may be generic for the detection of proteins, phosphopeptides, nucleic acids, polypeptides and the like. The detection methods may be directed towards the scoring of a presence or absence of one or more biomarker molecules or may be useful in the detection of expression levels.

The detection methods can be divided into two categories herein referred to as in situ methods or screening methods. The term in situ method refers to the detection of protein, phosphopeptide, and/or nucleic acid molecules in a sample wherein the structure of the sample has been preserved. This may thus be a biopsy (e.g., a heart biopsy) wherein the structure of the tissue is preserved. In situ methods are generally histological i.e. microscopic in nature and include but are not limited to methods such as: in situ hybridization techniques and in situ PCR methods.

Screening methods generally employ techniques of molecular biology and most often require the preparation of the sample material in order to access the nucleic acid and/or polypeptide molecules to be detected. Screening methods include, but are not limited to methods such as: Array systems, affinity matrices, Northern blotting and PCR techniques, such as real-time quantitative RT-PCR.

### Probe

One aspect of the present disclosure is to provide a probe which can be used for the detection of a protein, phosphopeptide, nucleic acid and/or polypeptide molecule as defined herein. A probe as defined herein is a specific sequence of a nucleic acid and/or polypeptide used to detect nucleic acids and/or polypeptides by hybridization. For example, a nucleic acid is also here any nucleic acid, natural or synthetic such as DNA, RNA, LNA or PNA. A probe may be labeled, tagged or immobilized or otherwise modified according to the requirements of the detection method chosen. A label or a tag is an entity making it possible to identify a compound to which it is associated. It is within the scope of the present disclosure to employ probes that are labeled or tagged by any means known in the art such as but not limited to: radioactive labeling, fluorescent labeling and enzymatic labeling. Furthermore the probe, labeled or not, may be immobilized to facilitate detection according to the detection method of choice and this may be accomplished according to the preferred method of the particular detection method.

### Detection Methods

Another aspect of the present disclosure regards the detection of nucleic acid and/or polypeptide molecules by any method known in the art. In the following are given examples of various detection methods that can be employed for this purpose, and the present disclosure includes all the mentioned methods, but is not limited to any of these.

### In Situ Hybridization

In situ hybridization (ISH) applies and extrapolates the technology of nucleic acid and/or polypeptide hybridization to the single cell level, and, in combination with the art of cytochemistry, immunocytochemistry and immunohistochemistry, permits the maintenance of morphology and the identification of cellular markers to be maintained and identified, allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). DNA ISH can be used to determine the structure of chromosomes and the localization of individual genes and optionally their copy numbers. Fluorescent DNA ISH (FISH) can for example be used in medical diagnostics to assess chromosomal integrity. RNA ISH is used to assay expression and gene expression patterns in a tissue/across cells, such as the expression of miRNAs/nucleic acid molecules. Sample cells are treated to increase their permeability to allow the probe to enter the cells, the probe is added to the treated cells, allowed to hybridize at pertinent temperature, and then excess probe is washed away. A complementary probe is labeled with a radioactive, fluorescent or antigenic tag, so that the probe's location and quantity in the tissue can be determined using autoradiography, fluorescence microscopy or immunoassay, respectively. The sample may be any sample as herein described. The probe is likewise a probe according to any probe based upon the biomarkers mentioned herein.

An aspect of the present disclosure includes the method of detection by in situ hybridization as described herein.

### In Situ PCR

In situ PCR is the PCR based amplification of the target nucleic acid sequences prior to ISH. For detection of RNA, an intracellular reverse transcription (RT) step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR the cells are cytocentrifugated onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens. Detection of intracellular PCR-products is achieved by one of two entirely different techniques. In indirect in situ PCR by ISH with PCR-product specific probes, or in direct in situ PCR without ISH through direct detection of labeled nucleotides (e.g. digoxigenin-11-dUTP, fluorescein-dUTP, ³H-CTP or biotin-16-dUTP) which have been incorporated into the PCR products during thermal cycling.

An embodiment of the present disclosure concerns the method of in situ PCR as mentioned herein above for the detection of nucleic acid molecules as detailed herein.

### Microarray

A microarray is a microscopic, ordered array of nucleic acids, proteins, small molecules, cells or other substances that enables parallel analysis of complex biochemical samples. A DNA microarray consists of different nucleic acid probes, known as capture probes that are chemically attached to a solid substrate, which can be a microchip, a glass slide or a microsphere-sized bead. Microarrays can be used e.g. to measure the expression levels of large numbers of polypeptides/proteins/nucleic acids simultaneously.

Microarrays can be fabricated using a variety of technologies, including printing with fine-pointed pins onto glass slides, photolithography using pre-made masks, photolithography using dynamic micromirror devices, ink jet printing, or electrochemistry on microelectrode arrays.

An aspect of the present disclosure regards the use of microarrays for the expression profiling of biomarkers in conditions such as ischemic and/or non-ischemic heart failure. For this purpose, and by way of example, RNA is extracted from a cell or tissue sample, the small RNAs (18-26-nucleotide RNAs) are size-selected from total RNA using denaturing polyacrylamide gel electrophoresis (PAGE). Then oligonucleotide linkers are attached to the 5′ and 3′ ends of the small RNAs and the resulting ligation products are used as templates for an RT-PCR reaction with 10 cycles of amplification. The sense strand PCR primer has a Cy3 fluorophore attached to its 5′ end, thereby fluorescently labeling the sense strand of the PCR product. The PCR product is denatured and then hybridized to the microarray. A PCR product, referred to as the target nucleic acid that is complementary to the corresponding RNA capture probe sequence on the array will hybridize, via base pairing, to the spot at which the capture probes are affixed. The spot will then fluoresce when excited using a microarray laser scanner. The fluorescence intensity of each spot is then evaluated in terms of the number of copies of a particular biomarker, using a number of positive and negative controls and array data normalization methods, which will result in assessment of the level of expression of a particular biomarker.

Several types of microarrays can be employed such as spotted oligonucleotide microarrays, pre-fabricated oligonucleotide microarrays or spotted long oligonucleotide arrays.

In spotted oligonucleotide microarrays the capture probes are oligonucleotides complementary to nucleic acid sequences. This type of array is typically hybridized with amplified.

PCR products of size-selected small RNAs from two samples to be compared that are labeled with two different fluorophores. Alternatively, total RNA containing the small RNA fraction is extracted from the abovementioned two samples and used directly without size-selection of small RNAs, and 3′ end labeled using T4 RNA ligase and short RNA linkers labeled with two different fluorophores. The samples can be mixed and hybridized to one single microarray that is then scanned, allowing the visualization of up-regulated and down-regulated biomarker genes in one go. The downside of this is that the absolute levels of gene expression cannot be observed, but the cost of the experiment is reduced by half. Alternatively, a universal reference can be used, comprising of a large set of fluorophore-labeled oligonucleotides, complementary to the array capture probes.

In pre-fabricated oligonucleotide microarrays or single-channel microarrays, the probes are designed to match the sequences of known or predicted biomarkers. There are commercially available designs that cover complete genomes from companies such as Affymetrix, or Agilent. These microarrays give estimations of the absolute value of gene expression and therefore the comparison of two conditions requires the use of two separate microarrays.

Spotted long oligonucleotide arrays are composed of 50 to 70-mer oligonucleotide capture probes, and are produced by either ink jet or robotic printing. Short Oligonucleotide Arrays are composed of 20-25-mer oligonucleotide probes, and are produced by photolithographic synthesis (Affymetrix) or by robotic printing. More recently, Maskless Array Synthesis from NimbleGen Systems has combined flexibility with large numbers of probes. Arrays can contain up to 390,000 spots, from a custom array design.

An embodiment of the present disclosure concerns the method of microarray use and analysis as described herein.

### PCR

The terms “PCR reaction,” “PCR amplification,” “PCR,” “pre-PCR,” “Q-PCR,” “real-time quantitative PCR” and “real-time quantitative RT-PCR” are interchangeable terms used to signify use of a nucleic acid amplification system, which multiplies the target nucleic acids being detected. Examples of such systems include the polymerase chain reaction (PCR) system and the ligase chain reaction (LCR) system. Other methods recently described and known to the person of skill in the art are the nucleic acid sequence based amplification and Q Beta Replicase systems. The products formed by said amplification reaction may or may not be monitored in real time or only after the reaction as an end-point measurement.

### Real-Time Quantitative RT-PCR

Real-time quantitative RT-PCR is a modification of polymerase chain reaction used to rapidly measure the quantity of a product of polymerase chain reaction. It is preferably done in real-time, thus it is an indirect method for quantitatively measuring starting amounts of DNA, complementary DNA or ribonucleic acid (RNA). This is commonly used for the purpose of determining whether a genetic sequence is present or not, and if it is present the number of copies in the sample. There are 3 methods which vary in difficulty and detail. Like other forms of polymerase chain reaction, the process is used to amplify DNA samples, using thermal cycling and a thermostable DNA polymerase.

The three commonly used methods of quantitative polymerase chain reaction are through agarose gel electrophoresis, the use of SYBR Green, a double stranded DNA dye, and the fluorescent reporter probe. The latter two of these three can be analyzed in real-time, constituting real-time polymerase chain reaction method.

Agarose gel electrophoresis is the simplest method, but also often slow and less accurate then other methods, depending on the running of an agarose gel via electrophoresis. It cannot give results in real time. The unknown sample and a known sample are prepared with a known concentration of a similarly sized section of target DNA for amplification. Both reactions are run for the same length of time in identical conditions (preferably using the same primers, or at least primers of similar annealing temperatures). Agarose gel electrophoresis is used to separate the products of the reaction from their original DNA and spare primers. The relative quantities of the known and unknown samples are measured to determine the quantity of the unknown. This method is generally used as a simple measure of whether the probe target sequences are present or not, and rarely as ‘true’ Q-PCR.

Using SYBR Green dye is more accurate than the gel method, and gives results in real time. A DNA binding dye binds all newly synthesized double stranded (ds)DNA and an increase in fluorescence intensity is measured, thus allowing initial concentrations to be determined. However, SYBR Green will label all dsDNA including any unexpected PCR products as well as primer dimers, leading to potential complications and artifacts. The reaction is prepared as usual, with the addition of fluorescent dsDNA dye. The reaction is run, and the levels of fluorescence are monitored; the dye only fluoresces when bound to the dsDNA. With reference to a standard sample or a standard curve, the dsDNA concentration in the PCR can be determined.

The fluorescence reporter probe method is the most accurate and most reliable of the methods. It uses a sequence-specific nucleic acid based probe so as to only quantify the probe sequence and not all double stranded DNA. It is commonly carried out with DNA based probes with a fluorescent reporter and a quencher held in adjacent positions, so-called dual-labeled probes. The close proximity of the reporter to the quencher prevents its fluorescence; it is only on the breakdown of the probe that the fluorescence is detected. This process depends on the 5′ to 3′ exonuclease activity of the polymerase involved. The real-time quantitative PCR reaction is prepared with the addition of the dual-labeled probe. On denaturation of the double-stranded DNA template, the probe is able to bind to its complementary sequence in the region of interest of the template DNA (as the primers will too). When the PCR reaction mixture is heated to activate the polymerase, the polymerase starts synthesizing the complementary strand to the primed single stranded template DNA. As the polymerization continues it reaches the probe bound to its complementary sequence, which is then hydrolysed due to the 5′-3′ exonuclease activity of the polymerase thereby separating the fluorescent reporter and the quencher molecules. This results in an increase in fluorescence, which is detected. During thermal cycling of the real-time PCR reaction, the increase in fluorescence, as released from the hydrolysed dual-labeled probe in each PCR cycle is monitored, which allows accurate determination of the final, and so initial, quantities of DNA.

Any method of PCR that can determine the expression of a nucleic acid molecule as defined herein falls within the scope of the present disclosure. A preferred embodiment of the present disclosure includes the real-time quantitative RT-PCR method, based on the use of either SYBR Green dye or a dual-labeled probe for the detection and quantification of nucleic acids according to the herein described.

### Northern Blot Analysis

An aspect of the present disclosure includes the detection of the nucleic acid molecules herein disclosed by techniques such as Northern blot analysis. Many variations of the protocol exist.

The following examples are offered by way of illustration and not by way of limitation.

### EXAMPLES

### Example 1

### Human Cardiac Tissue Acquisition and Tissue Repository

Human myocardium was acquired from the left ventricular (LV) free wall of explanted ischemic failing (IF) or non-ischemic failing (NIF) hearts during cardiac transplantation. Non-failing (NF) left ventricular tissue was acquired from donors whose hearts were suitable for transplantation but were not used because a suitable recipient could not be found. Explanted hearts from transplant recipients were arrested with cold blood cardioplegia, while unused donor hearts were arrested with cold Celsior solution. After explantation, transmural tissue samples 1-2 mm thick were obtained from the anterolateral LV free wall and immediately flash frozen in liquid nitrogen and stored in a −80° C. freezer. In the ischemic hearts, the area of infarct was identified at the time of tissue procurement and only sites remote from the infarct with grossly transmural muscle and minimal scar were used in this study.

### Sample Preparation for Mass Spectrometry

Heart tissue samples were homogenized in 1 mL TRIzol (Life Technologies, Grand Island N.Y.) per 0.1 mg heart tissue as previously described.⁹ After tissue homogenization using an electric homogenizer (BioSPEC Products Inc., Model 985-370) and centrifugation, 0.8 mL of the supernatant fraction was subjected to phase separation by addition of 0.2 mL chloroform. Following centrifugation, protein was precipitated from the organic layer by addition of 1.2 mL methanol. After washing and sonication of the pellet, the protein precipitate was recovered by centrifugation and re-suspended in 0.2 mL of 0.25% w/v mass spectrometry (MS)-compatible detergent (RapiGest, Waters Corp., Milford, Mass.) in 50 mM Ammonium Bicarbonate pH 8.0. A 625 μg aliquot of protein (per sample) was subjected to reduction (10 mM dithiothreitol, 80° C. for 30 min), alkylation (20 mM iodoacetamide, RT in dark for 1 h) followed by overnight proteolysis with 1:50 w/w sequencing grade trypsin (Promega, Madison, Wis.) at 37° C. A 25 μg aliquot from each sample was used for unenriched proteomic analysis of protein expression in the heart tissue. The 25 μg unenriched proteomics profiling aliquot was acidified to 1% v/v final Trifluoroacetic acid (TFA), heated to 60° C. for 2 h, and spiked with 1.25 pmol ADH1_YEAST digest (Massprep standard, Waters Corporation) as a surrogate standard prior to analysis.

The remaining 600 μg of protein was utilized for spin-column based phosphopeptide enrichment and LC/MS/MS analysis of the phosphoproteome. Prior to phosphopeptide enrichment, each sample was spiked with trypsin digested bovine alpha-casein at 30 fmol per μg/protein lysate for use as a surrogate standard. These samples were then enriched for phosphopeptides using an in-house packed TiO₂ spin column as previously described¹⁰. Briefly, samples were dried using vacuum centrifugation and re-suspended in 100 μL 80% acetonitrile, 50 mg/mL MassPrep enhancer (Waters Corp.), 1% TFA (pH 2.5). Samples were then loaded onto a TiO₂ column containing approximately 12 mg TiO₂ resin (Protea Biosciences Group, Inc., Morgantown, W. Va.) which were subsequently washed with 400 μL 80% acetonitrile, 50 mg/mL MassPrep enhancer, 1% TFA (pH 2.5) and then 400 μL 80% acetonitrile, 1% TFA (pH 2.5). Phosphopeptides were eluted using 200 μL 5% aqueous ammonia, 20% acetonitrile (pH 10.5) and were immediately acidified with neat formic acid down to pH 3.5. Samples were dried using vacuum centrifugation and then re-suspended in 2% acetonitrile, 0.1% TFA, 10 mM citric acid (pH 2.5) prior to LC/MS/MS analysis.

### LC/MS/MS Data Collection

The sample cohort was randomized prior to LC/MS/MS analysis. Peptide digests obtained from each of the samples were analyzed in a label-free quantitative fashion using a nanoAcquity UPLC system coupled to a Synapt HDMS mass spectrometer (Waters Corp, Milford, Mass.) for unenriched peptide analyses and an LTQ Orbitrap XL (Thermo Fisher Scientific, Waltham, Mass.) for phosphopeptide analyses. For proteomics or unenriched samples, 1 μg of peptides were first trapped at 20 μL/min for 2 min in 99.9% water with 0.1% v/v formic acid on a 20 μm×180 mm Symmetry C18 column. Peptides were eluted from the trapping column onto a 75 μm×250 mm column with 1.7 μm C18 BEH particles (Waters, Corp.). Peptide separations were accomplished using a 90-min gradient of 5 to 40% acetonitrile (0.1% formic acid) at a flow rate of 0.3 μl/min and a 45° C. column temperature. MS and MS/MS data was collected using data-independent analysis (MS^{E}) for simultaneous peptide quantification and identification using a 0.9 s cycle time, alternating between MS (low collision energy—6 V) and MS/MS (high collision energy ramp—15 to 40 V). These qualitative/quantitative analyses were followed by an additional, supplementary qualitative LC/MS/MS experiment using data-dependent analysis (DDA) with a 0.9 s MS scan followed by MS/MS acquisition on the ‘top 3’ ions with charge greater than 1. The MS/MS acquisition for each ion used an isolation window of approximately 3 Da, a maximum of 4 s per precursor, and dynamic exclusion for 120 s (within 1.2 Da).

LTQ-Orbitrap phosphopeptide analysis was performed using the same nanoscale capillary LC column hardware and LC system that was employed for unenriched proteome analysis except that the gradient was modified by increasing the trapping time to 5 min and then a gradient hold at 5% acetonitrile (0.1% formic acid) for 5 min prior to initiating the linear gradient from 5 to 40% acetonitrile (0.1% formic acid). MS data were acquired in the Orbitrap from m/z 400-2000 with r=60,000 at m/z 400 and a target AGC setting of 1⁶ ions. The qualitative/quantitative LC/MS/MS analyses spectra utilized DDA for the ‘top 3’ precursor ions and supplementary qualitative LC/MS/MS analyses used DDA for the ‘top 10’ precursor ions. Peptide fragmentation was performed in the LTQ linear ion trap, with a CID energy setting of 35% and a dynamic exclusion of 60 s.

### LC-MS Data Processing

Robust peak detection and label-free alignment of individual peptides across all sample injections, was performed using the commercial package Rosetta Elucidator® v3.3 (Rosetta Biosoftware, Inc., Seattle, Wash.) with PeakTeller algorithm¹¹ as previously described.^{12,10,13,14,15} Unenriched and phospho-enriched proteomics datasets were independently aligned on the basis of their accurate mass and retention time. After alignment and annotation, chromatographic peak intensities belonging to the same precursor mass in the aligned chromatograms were then used to calculate the relative peptide and protein abundance on a per-sample basis. MS^{E} from the Q-ToF was used exclusively for peptide quantitation of unenriched proteomes. Protein intensities for each sample were calculated as the simple sum of the peptide intensity values. Phosphopeptide quantitation was performed on the LTQ-Orbitrap XL instrument at the peptide level from the qualitative/quantitative acquisitions.

Both MS/MS DDA and MS^{E} were used to generate peptide identifications for the unenriched analysis, and DDA exclusively for phosphopeptides. For DDA acquisition files, .mgf searchable files were produced in Rosetta Elucidator®, and searches were then submitted to and retrieved from the Mascot v2.2 (Matrix Sciences, Boston, Mass.) search engine in an automated fashion. For MS^{E} data, ProteinLynx Global Server 2.4 (Waters Corp.) was used to generate searchable files, which were then submitted to the IdentityE search engine (Waters Corp.)^{16,17} and results files were then imported back into Elucidator®.

Both DDA and MS^{E} data were searched against the Uniprot/reviewed database with human taxonomy with full 1× reverse database appended for peptide false discovery rate determination. The final database contained 40,668 sequences including reverse entries. Q-ToF data (unenriched proteome) used a precursor ion mass tolerance of 20 ppm for both PLGS and Mascot database (DB) searches, and a product ion tolerance of 0.1 Da for Mascot and 40 ppm for PLGS. Orbitrap data (phosphoproteomics) was searched with Mascot using 10 ppm precursor and 0.8 Da product ion tolerances. Enzyme specificity was set to fully tryptic and allowed for up to 2 missed cleavages, with the exception that semi-tryptic specificity was allowed for Mascot (DDA) searches of unenriched data. Carbamidomethyl cysteine was included as a fixed modification, and variable modifications were allowed for including oxidized methionine and deamidated asparagine and glutamine. Additionally, for phosphopeptide enriched mixtures, variable phosphorylation on serine, threonine, and tyrosine was allowed.

The spectra were submitted for database searching and results were imported into ELUCIDATOR®. To enable global spectra scoring across results from both search engines these search results were concurrently validated using the PeptideProphet and ProteinProphet algorithms in ELUCIDATOR® using an independent reverse decoy database validation.^{18,19} Annotation was performed to achieve a maximum 1% FDR at the peptide level, which corresponded to a minimum PeptideProphet score of 0.6. Each peptide identified was allowed to be assigned to only a single protein entry, and these assignments were made by ProteinProphet according to the rules of parsimony. For the phosphoproteomic experiments, a mascot ion score of 26 was applied to achieve a spectral false discovery rate of 1.0%.

### Statistical Analysis

Basic statistical analysis was performed on both the unenriched (protein-level) and phosphopeptide (peptide-level) datasets in order to obtain candidate (phospho) proteins which were differentially expressed. Fold-changes were calculated for each failing group versus non-failing control, as the ratio of the average intensity between the groups; directionality of the ratio was established that positive fold-changes mean up-regulated in failing versus nonfailing control, and negative fold-changes mean down-regulated in failing versus nonfailing. P-values were calculated using an error-weighted ANOVA with Benjamini-Hochberg FDR correction for multiple hypotheses testing (Rosetta Elucidator v3.3). The input for this test was the protein-level data for unenriched analysis (intensity for all peptides summed per sample), or the peptide-level data for phosphopeptides, and the raw intensities were scaled to a normal distribution using the Error Model in Elucidator software prior to ANOVA. Fold-changes and p-values are shown for all proteins (Table 7) and phosphopeptides (Table 8). Statistical cutoffs for fold-change were established for proteins or phospho-peptides based on a power calculation using the average biological variation within each group. Using the protein average % CV (23%) or phosphopeptide % CV (54%) and 4 reps per group at a 95% confidence—minimum cutoffs were set to 2-fold (98% powering) or 6-fold (86% powering) for proteins or phosphopeptides, respectively. ANOVA p-value of 0.05 or less was required. The candidate differentially expressed proteins and phosphopeptides meeting these criteria are shown in Tables 2 and 3.

### Sample Preparation for Western Blot Analysis

Cryopreserved heart tissues (independent of the samples used for proteomics analysis) of each of the 12 hearts examined in the LC/MS/MS analysis were weighed and mechanically disrupted by mortar and pestle in liquid nitrogen. Pulverized heart tissues were suspended in a 5:1 volume-to-tissue weight of lysis buffer (1% IGEPAL CA-630, Sigma, 0.5% Deoxycholate, 2% SDS, 5 mM EDTA in 1×PBS) with protease and phosphatase inhibitor cocktail tablets (Roche Diagnostics, Indianapolis, Ind.). Samples were then pulse homogenized on ice with a handheld tissue tearor (BioSPEC Products Inc., Model 985-370). Homogenates were placed on ice for an additional 30 min followed by centrifugation for 30 min at 4° C. at 16,000×g (Heraeus Biofuge® Pico). Resulting supernatants were aliquoted and stored at −80° C. until analysis.

### Western Blot Analysis

Cardiac tissue homogenates were subjected to Bicinchoninic acid (BCA) assay (Pierce Biotechnology/Thermo Fisher Scientific, Rockford, Ill.) for protein quantification. Western Blot for protein immunodetection was performed using a modification⁸ The primary antibodies used in this study were the following: anti-Fetuin A (#5258 Cell Signaling Technologies, Danver, Mass.), anti-fibulin 1 (ab54652 Abcam, Cambridge, Mass.), anti-ceruloplasmin (ab8813, Abcam), anti-alpha 2 macroglobulin (ab58703, Abcam), anti-carbonic anhydrase I (ab6619-1, Abcam), anti-serum amyloid A (ab687, Abcam), anti-fibulin 2 (ab66333, Abcam), anti-AKT (#9272, Cell Signaling), anti-SMAD3 (ab28379, Abcam), anti-MMP14 (ab51074, Abcam), and anti-AHR (ab28698, Abcam).

The secondary antibodies used were horseradish peroxidase conjugated anti-rabbit IgG (GE Healthcare, UK), anti-mouse IgG (Pierce), anti-sheep IgG (ab6747, Abcam), or anti-goat IgG (Sigma-Aldrich, St. Louis, Mo.). Bands were visualized using an enhanced chemiluminescence Western blotting detection system (GE Healthcare Bio-Sciences, Piscataway, N.J.). Western blots were stripped and re-probed with anti-sarcomeric actin (Sigma, A2172). The intensity of the actin band signal was used for normalization. Proteins detected were quantitated by densitometry utilizing the Image J algorithm (National Institutes of Health, Bethesda, Md.).

### Bioinformatics Analysis of Protein Data Sets

Ingenuity Pathway Analysis (IPA, Winter 2012 Release) (Ingenuity Systems, Redwood City, Calif.) was used to classify the proteins according to primary function as well as for pathway analysis.

### Patient Samples

Left ventricular tissue from 12 male patients matched for age and race was used in this study (Table 1). The tissue was acquired from three groups (n=4 per group): explanted hearts from transplant patients with either ischemic cardiomyopathy (ischemic failing, IF) or non-ischemic cardiomyopathy (non-ischemic failing, NIF), or non-failing (NF) donor hearts not used for transplantation. Groups were closely matched for cardiac function and treatment history (Table 1). All patients with HF exhibited significantly lower ejection fractions (EF) compared to NF controls, which had normal left ventricular EF. All patients with HF received intravenous inotropic agents and intra-aortic balloon pump support, whereas only patients from the IF subset received prior coronary artery bypass surgery. In the NIF group, two patients were diagnosed with non-ischemic cardiomyopathy of unknown etiology, one patient had a viral cardiomyopathy, and the fourth patient developed HF secondary to valvular disease.

### Quantitative Analysis of Proteins in Non Failing and Failing Human Left Ventricles

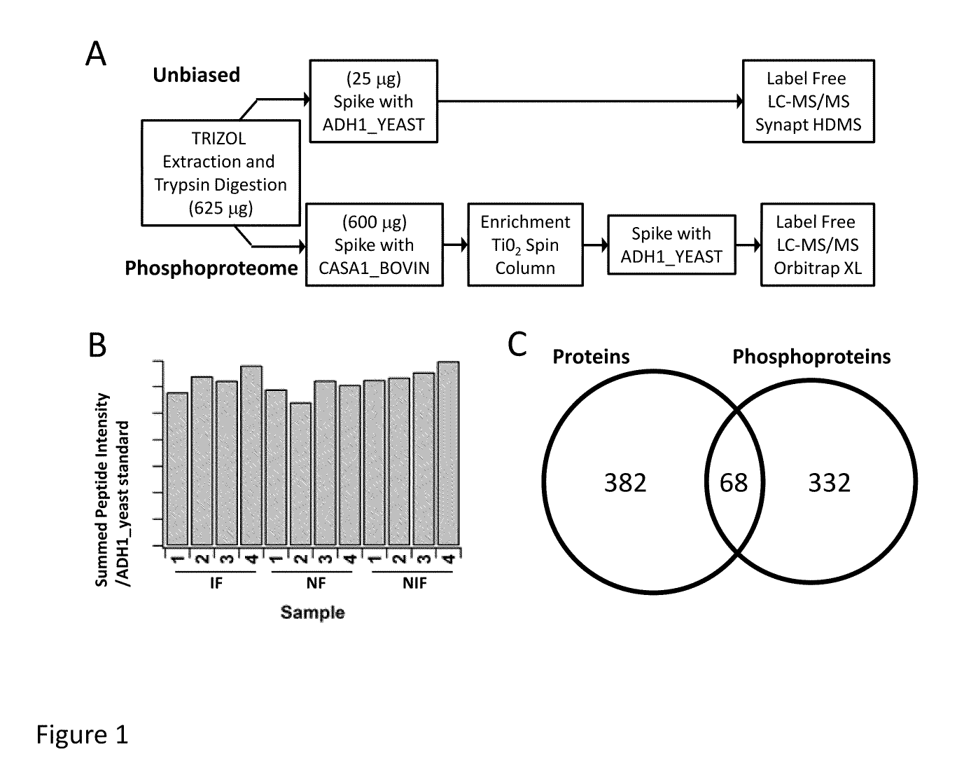

All heart tissue samples were subjected to quantitative analyses of the unenriched and TiO₂-enriched phosphoproteome following the workflow in FIG. 1A and outlined herein. The reproducibility of the unenriched analytical approach was validated with a spike-in of known concentration of yeast ADH1 digest as an internal standard. Consistent ADH1 abundance (7% coefficient of variation (CV)) across all 12 samples was observed (FIG. 1B).

A total of 850 proteins was identified cumulatively from the unenriched and phospho-enriched analyses. Peptide/protein identification and quantification by Rosetta Elucidator with Mascot and IdentityE search algorithms yielded expression data for 4,436 peptide annotations representing 450 proteins in the unenriched samples for which relative quantitation between samples was performed (FIG. 1C; Table 7). For phosphopeptides (Table 8), site specific quantitation for 823 phosphopeptides corresponding to 400 phosphorylated proteins (FIG. 1C) was determined. Only sixty-eight proteins overlapped between the observed unenriched proteome and the phosphoproteome (FIG. 1C). Whereas peptides and proteins identified were highly reproducible within any one preparation type, the uniqueness between the observed proteins in the unenriched and TiO₂-enriched approaches underscores the highly complementary nature of the two analysis methods. Tables 9 and 10 list the 68 proteins in common between the datasets and their respective fold changes and p-values.

### Principal Components Analysis

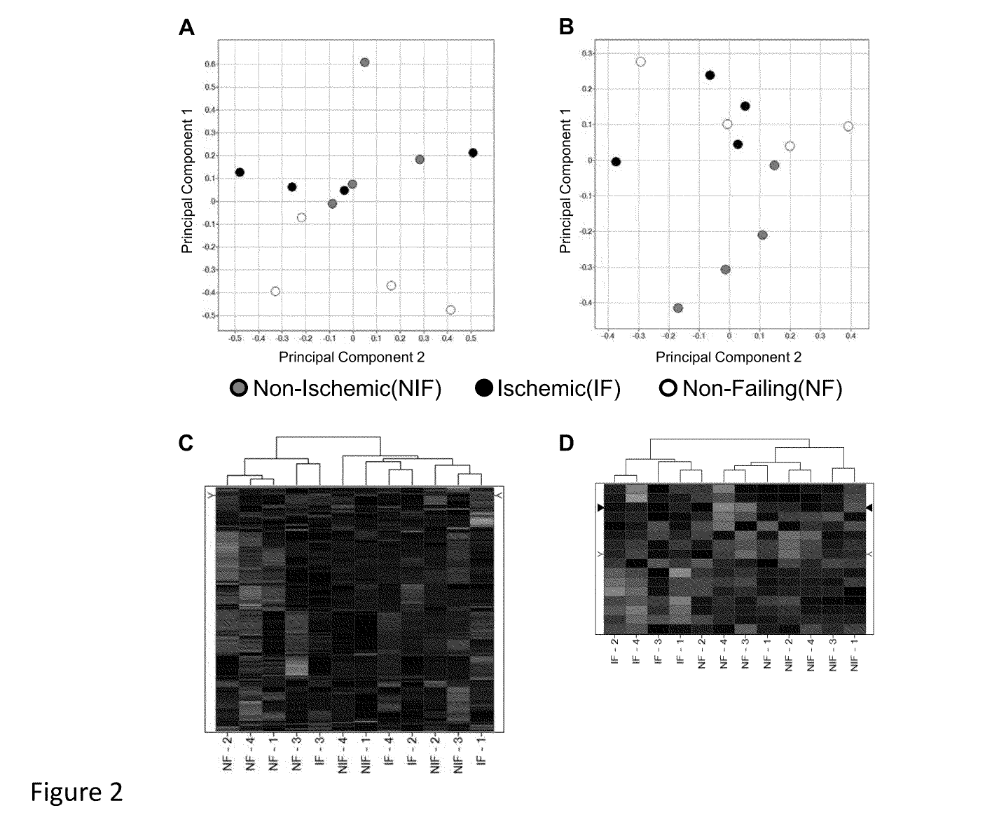

Using the expression data for the unenriched and phosphoproteomes, principal components analysis (PCA) was performed in order to observe any high-level differences between sample groups and to screen for outlier samples. Expression data was z-score transformed across all samples at the protein-level for unenriched samples (FIG. 2A) or at the peptide level for phospho-enriched samples (FIG. 2B) and principal components were calculated using Rosetta Elucidator v3.3. The two most prominent components (PC1 versus PC2) are plotted in FIGS. 2A and 2B, and no significant outliers are observed for either unenriched or phosphoproteomes. Additionally, some small separation in PC1 (y-axis) occurs for one group in both plots. Interestingly, the four non-failing (NF) samples separate from the failing samples along PC1 in unenriched proteomes (FIG. 2A). However, in the phosphoproteome, at least three of the four non-ischemic failing (NIF) appear unique along PC1 compared to the IF or NF groups (FIG. 2B). This global observation indicates that the unenriched proteome may best separate failing from nonfailing hearts, whereas phosphorylation status may play a larger role in distinguishing ischemic versus non-ischemic failure.

### Differential Expression Analysis and Statistically Significant Proteins

Overall, the unenriched proteome shows primarily differences between non-failing (NF) and the two failing groups, while the phosphoproteome seems to more readily differentiate ischemic failure (IF) from non-ischemic failure (NIF) or non-failing hearts (NF). Tables 2 and 3 contain the list of significant, differentially expressed proteins and phosphopeptides. Using these candidate molecules, unbiased 2D hierarchical clustering analysis was performed at the protein-level (FIG. 2C) or phosphopeptide level (FIG. 2D) in order to observe the overall expression pattern of these molecules in each individual sample and determine how these candidates seem to segregate the failing and nonfailing hearts. FIG. 2C shows clear differentiation between failing (NIF, IF) and nonfailing (NF) hearts using proteins from the unenriched analyses, while the NIF and IF samples are not clearly differentiated. The phosphopeptides (FIG. 2D), however, show a potentially more robust differentiation between all three groups.

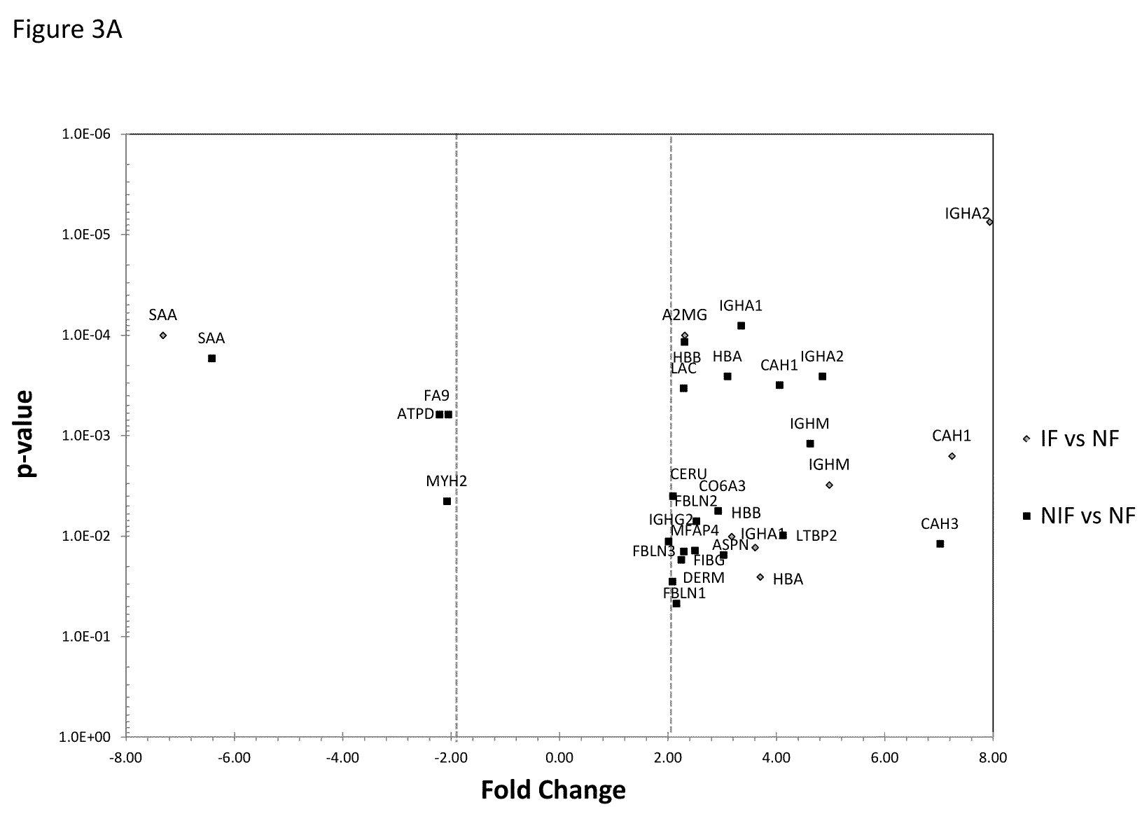

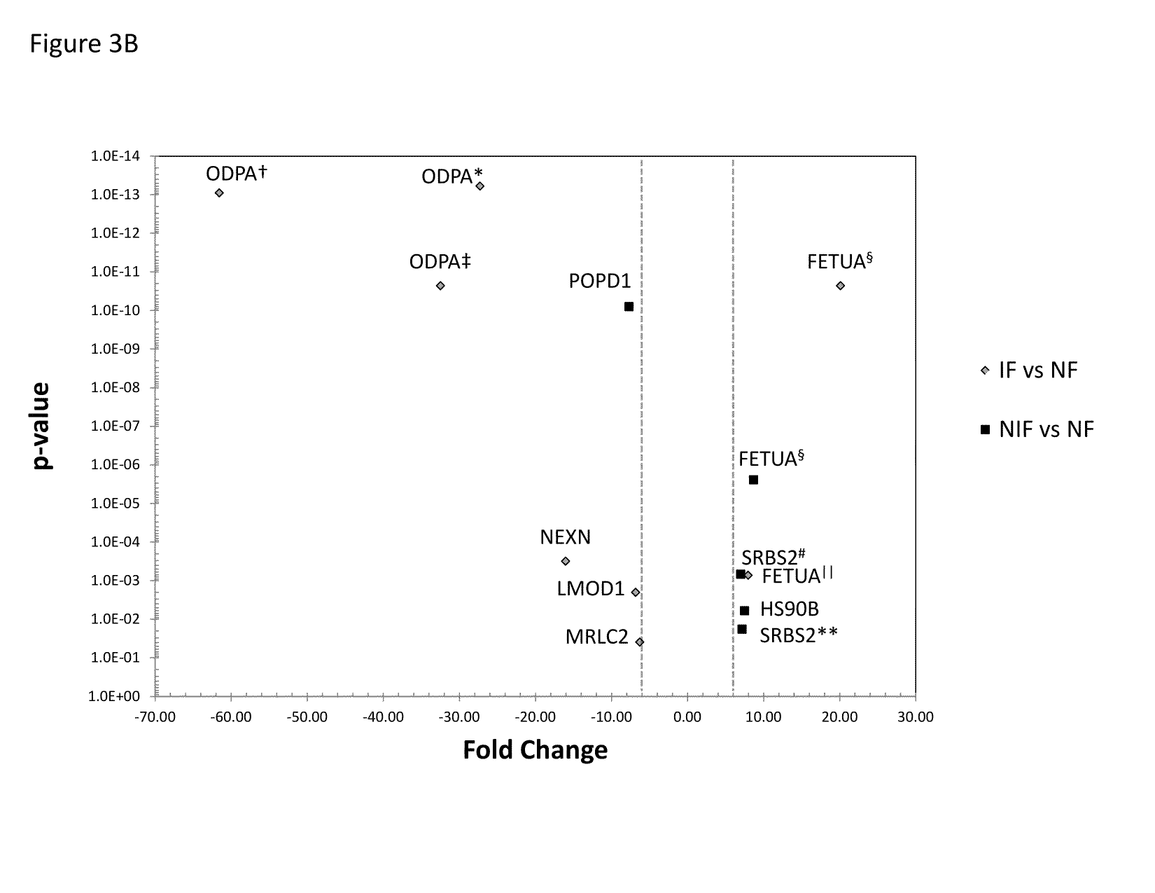

The unenriched comparison of failing to NF hearts showed thirty-one distinct proteins that were represented by at least two high confidence peptides and a significant (ANOVA, p<0.05) fold change of at least two (FIG. 3A and Table 2A and B). Four proteins (serum amyloid A, Factor IX, mitochondrial ATP synthase subunit delta, myosin-2) were decreased in abundance in the failing hearts, while the remaining proteins were increased in abundance. Alpha 2 macroglobulin was the only protein distinct between IF and NIF. All other proteins that changed in abundance in IF samples were common to the proteins that changed in the NIF group. Among the identified proteins found to be significantly changed in failing human myocardial tissue were extracellular matrix proteins, immunoglobulin subunits, secreted glycoproteins, coagulation proteins, hemoglobin subunits, ceruloplasmin, carbonic anhydrase, and serum amyloid A. Peptides from the fibulin family of extracellular matrix proteins (fibulin 1, fibulin2, and latent transforming growth factor beta binding protein 2 (fibulin 3)) were consistently up-regulated across all four NIF samples.

The comparison of failing to NF hearts found thirteen phosphopeptides with at least a 6-fold change and ANOVA p-value <0.05 (FIG. 3B and Tables 3A and B). With the exception of Leiomodin-1, the total level of each of these proteins was also quantified in the unenriched dataset (Table 4), enabling independent verification of whether protein abundance difference was responsible for the change in phosphopeptide abundance. Using the combination of the datasets, it was concluded that the phosphorylation level differences of at least 12 of the 13 statistically significant phosphopeptides were due to differential phosphorylation and not to differential protein abundances.

The phosphopeptides that were differentially expressed in IF versus NF heart tissue were mostly decreased in abundance (6/8; Table 3A), whereas 4 out of the 5 phosphopeptides that were different between NIF and NF groups were higher in the NIF hearts (Table 3B). α-2HS glycoprotein precursor (FETUA) was the only protein whose phosphorylation profile was augmented in both the NIF and IF. Interestingly, ischemic failing hearts had a marked decrease in the phosphorylation of three modulatory serine sites (S231, S293, S300, Table 3 and FIG. 3B) of pyruvate dehydrogenase (ODPA).

### Validation Western Blot Analysis:

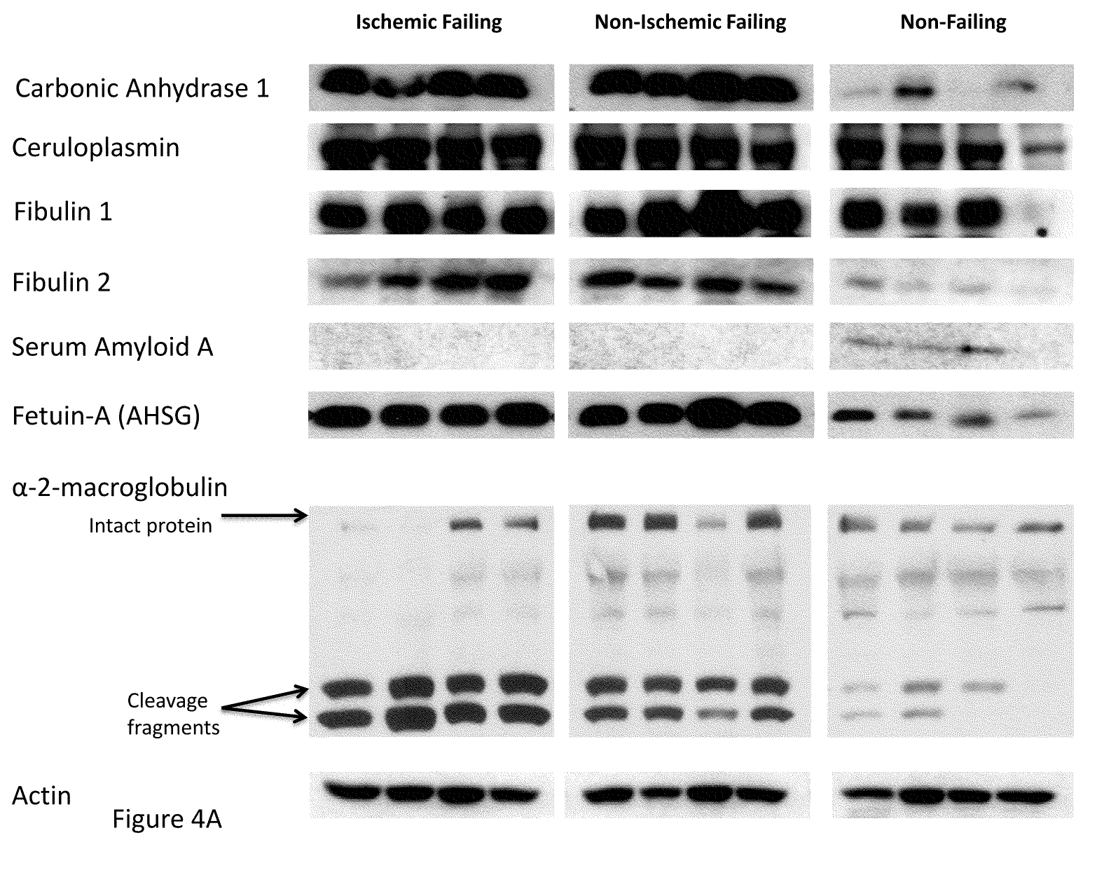

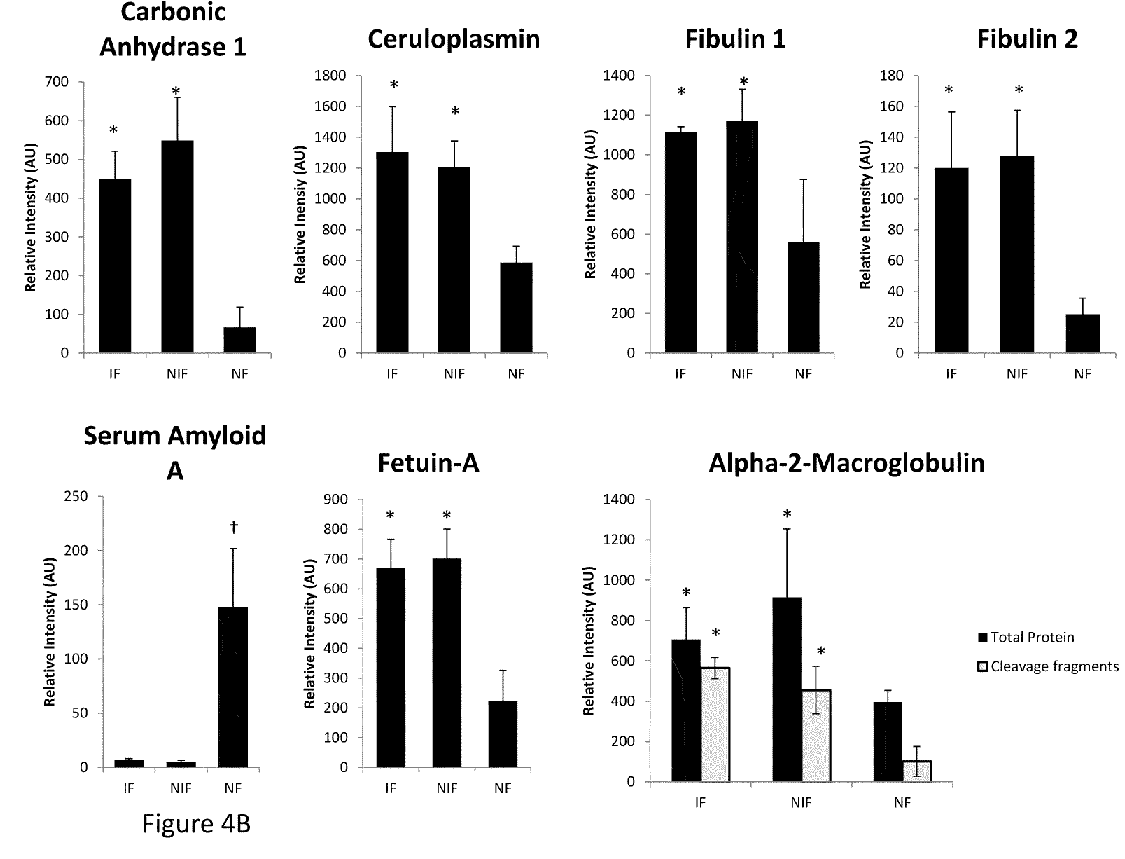

Western blots performed from independently prepared tissue homogenates from cardiac tissue from the same 12 patients was used to validate the proteomic data (FIG. 4A). Quantitation of Western Blot data is shown in FIG. 4B. These Western blots confirmed increased levels of carbonic anhydrase, ceruloplasmin, Fibulin1, and Fibulin2 in both IF and NIF tissue compared to the NF tissue. A decrease in serum amyloid A in both the NIF and IF compared to NF tissue was also confirmed. Alpha 2-HS glycoprotein (FETUA) was also increased in both IF and NIF tissue compared to NF controls. This increase was also observed in the mass spectrometry proteomic analysis, but the fold difference in FETUA levels did not reach our statistical cutoff. In agreement with the mass spectrometry data, Western blot analysis demonstrated that the overall abundance of α2-Macroglobulin (α2M) was increased in both IF and NIF heart tissues. Interestingly, a cleaved version of α2M was the prevalent form of α2M in both NIF and IF hearts whereas the full-length α2M was the prevalent species in NF heart tissue (FIGS. 4A and 4B).

### Ingenuity Analysis

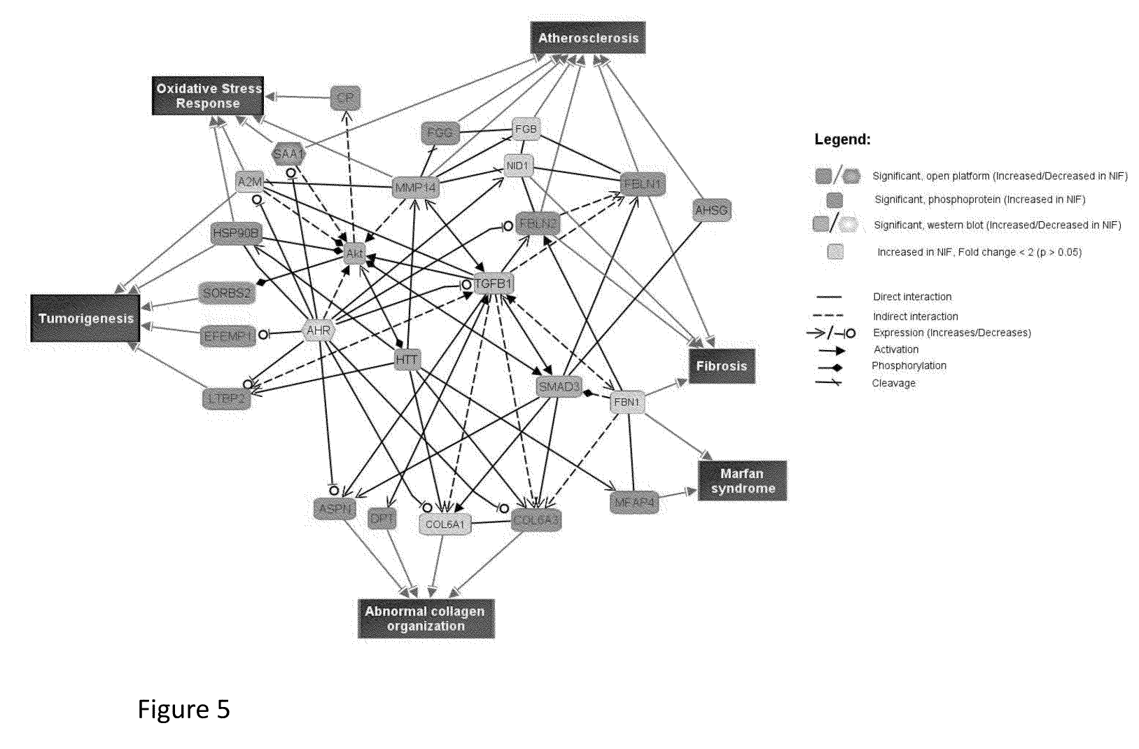

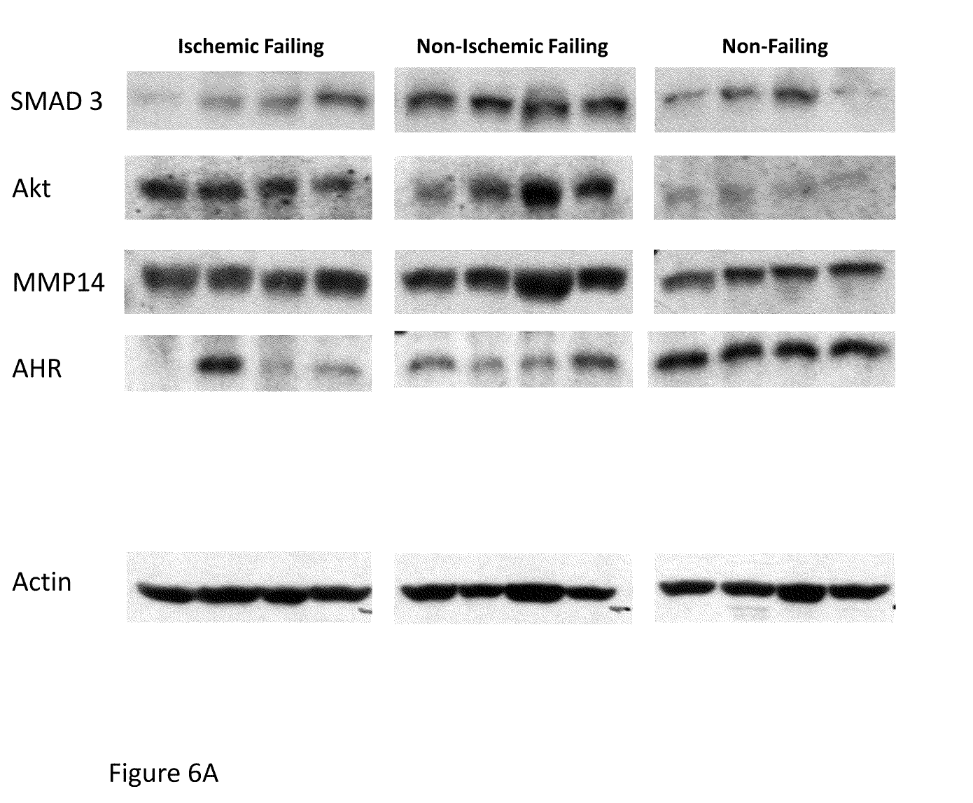

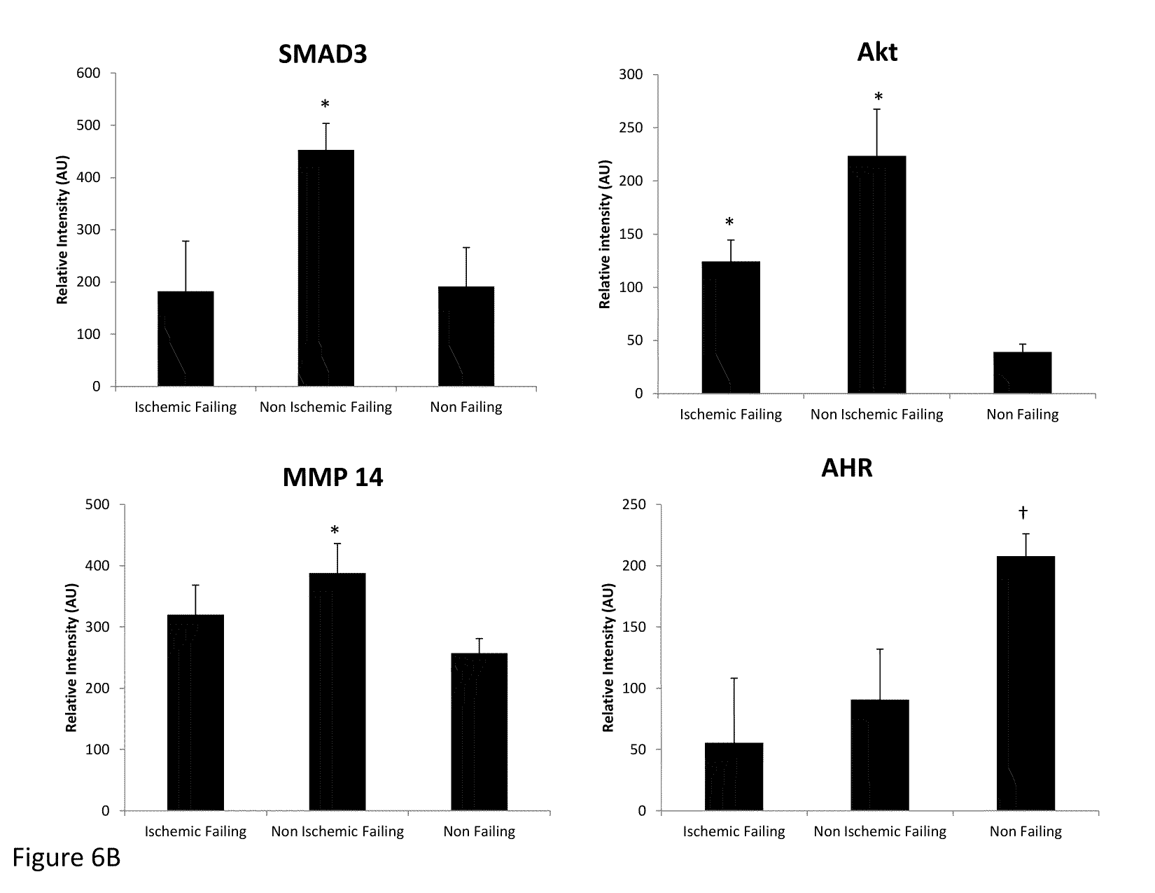

The interaction network of proteins (Ingenuity Pathway Analysis) differentially expressed in the NIF tissue is shown in FIG. 5. Many of the proteins differentially expressed or phosphorylated in the NIF hearts were glycoproteins, proteoglycans and structural proteins, all components of the extracellular matrix (ECM). Other proteins that were found to be significantly changed in the NIF group, but with less than a two-fold change, were also noted to be part of this network (FIG. 5). IPA was used to establish that many of the proteins identified in this study were downstream targets of AKT, SMAD3, matrix metalloprotease 14 and/or aryl hydrocarbon receptor (AHR), all of which have been implicated in cardiac remodeling.²⁰⁻²³ Western blot analysis was used to establish that these more central proteins (AKT, SMAD3, and AHR) were also differentially expressed in non-ischemic HF (FIGS. 6A and B).

HF remains for the most part an irreversible disease with limited treatment options; however, early diagnosis is paramount to improved patient prognosis making better and earlier means of therapeutics possible. Mass spectrometry based proteomics is a technology useful to obtain qualitative and quantitative information on hundreds to thousands of proteins among large sample cohorts. Proteomic based studies on cardiac diseases are already enabling biomarker and therapeutics discovery.²⁴⁻²⁶

Many proteomics studies of human hearts have been limited by the quality of tissue and associated clinical data. In this study, care was taken to standardize tissue procurement and storage, as well as match them clinically. Because of the robustness of the repository, we were able to perform secondary validation on samples from the same heart independent of the samples used for proteomics.

In this study 850 proteins or residue specific phosphorylation sites were identified and quantitated that could be compared across the 12 human samples. Based on the significant proteins observed, the derangements in inflammatory, metabolic, and extracellular remodeling pathways underlying HF are better characterized.

A number of proteins that distinguish the failing from NF samples are abundant in the circulation, although they can also be identified in the tissue interstitium. The abundance of serum proteins in the failing heart might be due in part to differences in the cardiac acquisition protocol. Although all hearts are arrested prior to acquisition, NF donor hearts are acquired following administration of Celsior solution, whereas failing explanted hearts are arrested using blood cardioplegia. This subtle difference in acquisition protocol may have impacted the levels of serum proteins remaining in each sample type. However, when the differential levels of two of the most abundant serum proteins (albumin and transferrin)^{27,28} (Table 7), were examined, serum albumin was not differently expressed according to the criteria used (<2 fold difference) and transferrin was not even detected, suggesting that these acquisition variables are unlikely to have significantly influenced the levels of these proteins in the hearts. Five of the statistically significant proteins in Table 2 are components of immunoglobulins (2 from IgG, 2 from IgA, and 1 from IgM). While 40% the total immunoglobulin proteins in the unenriched data set (Table 7) were from IgG, only 25% of these IgG components were up-regulated in failing hearts and the remainder was unchanged. However, all of the IgA components identified (20% of the total immunoglobulin proteins in the unenriched data set) were increased in both IF and NIF hearts, suggesting IgA may be selectively up-regulated or sequestered in the failing hearts. In Celiac disease, which was demonstrated to be associated with 5% of autoimmune myocarditis or idiopathic dilated cardiomyopathy, there is a widespread deposition of IgA in tissues including the myocardium.²⁹ The exact significance of this up-regulation is unknown, but this observation of a significant increase of IgA in both type of HF suggests that global activation of the inflammatory pathway likely plays a role in the pathology of HF.

The increased presence of ceruloplasmin, as well as the differential phosphorylation of heat shock protein 90, further implicates the inflammatory pathway in the development of HF. Ceruloplasmin is an acute phase reactant which increases in inflammatory diseases and in acute coronary syndromes. Ceruloplasmin primarily functions as a transporter of copper, a metal which can directly damage cells as well as promote the development of reactive oxygen species (ROS).³⁰ Its involvement in copper regulation likely explains the association between ceruloplasmin and vascular disease.³⁰ Heat-shock protein 90 (HSP90) is also increased by ROS, and is thought to stabilize and regulate many cellular proteins in response to cellular insults.³¹ The increased expression of both of these proteins suggests that constant cellular stress and inflammation underlies the development of HF. Another acute phase reactant, serum amyloid A, was decreased in failing hearts. SAA is a marker of inflammation, and increased levels of SAA are associated with increased risk of cardiovascular disease.^{31,32,33} However, in healthy individuals, the serum concentration of SAA can increase over 1000-fold in response to infection or tissue damage.³³ Therefore, while one might expect SAA levels to be increased in HF, the events surrounding organ donation from relatively healthy individuals could have dramatically increased the levels of serum amyloid A, thus explaining these results.

The finding of increased α2-macroglobulin (α2M) cleavage products is particularly noteworthy. When exposed to a protease, α2M undergoes limited proteolysis, which exposes a “bait region” within its structure, trapping the protease and promoting its degradation.³⁴ In addition to proteases, α2M can, in its native form, bind to damaged proteins including those associated with protein deposition disorders, such as Alzheimer's disease and dialysis-related amyloidosis, in order to prevent cellular damage.³⁵ This broader protective function of α2M is lost, however, upon exposure to a protease.³⁵ Therefore, the increased levels of cleaved α2M in HF tissue could be protective attempts to limit the effects of harmful proteases. Conversely, the decreased ability of α2M to bind to and clear other damaging protein deposits accumulating from ongoing cardiomyocyte death could be contributing to the progression of HF. Whether a cause of—or response to—cellular damage, the cleaved form of alpha-2 macroglobulin could be developed into a biomarker for the progression of HF.

The failing heart, regardless of etiology (IF versus NIF), is characterized by a severe energy metabolism derangement.³⁶ In the normal, well-perfused heart, fatty acids provide 60-90% of the energy for ATP production, with the remaining 10-40% are derived from carbohydrate (glucose and lactate) oxidation.³⁷ In the failing heart, there is a switch to glucose as the preferential fuel source instead of fatty acids. A decrease in phosphorylation of pyruvate dehydrogenase E1 component subunit α (ODPA) at Ser231, 293, 300 was observed in ischemic failing heart (Table 3A and FIG. 3B), indicating a significant increase in the ODPA activity. This finding is supportive of the current dogma regarding the switch towards glucose, and suggests a mechanism by which this is occurring. It is still unclear if the shift in energy substrate observed in the ischemic heart represents a protective response to chronic ischemia or a maladaptive response that further stresses the ischemic heart.³⁸ Interestingly, stimulation of glucose oxidation has been shown to protect against acute myocardial infarction and reperfusion injury³⁹, but a chronic switch to glucose oxidation could lead to metabolic derangements and cellular damage.

The present study has used proteomic and pathway analysis to determine the more central molecules involved in the regulation of the identified proteins. Many of the differentially expressed or phosphorylated proteins are either components or regulators of the extracellular matrix (ECM). This analysis further establishes the importance of these central proteins in the development of HF, as well as suggests possible mechanisms through which they manifest their effect

These findings also demonstrate how proteomic and pathway analyses provide a more comprehensive understanding of human disease. TGFB1 is heavily involved in this network (FIG. 5). Furthermore, in whole tissue lysates (compared to only extracellular components), cellular proteins involved in ECM remodeling, including HSP90B and MMP14, were identified. Also identified was SORBS2, a membrane-bound cytoskeletal adaptor protein located in the Z-bands of myofibrils of cardiac muscle. Also, many of the significant proteins directly interact with the huntingtin protein. These analyses suggest that the huntingtin protein may play an important role in the development of HF through the regulation of both apoptosis and cardiac ECM remodeling.

### References

1. Roger et al. “American Heart Association Statistics C, Stroke Statistics S. Heart disease and stroke statistics—2012 update: A report from the American Heart Association” Circulation 2012; 125:e2-e220
2. Lloyd-Jones et al. “Framingham Heart S. Lifetime risk for developing congestive heart failure: The Framingham Heart Study” Circulation 2002; 106:3068-3072
3. Roger et al. “Executive summary: Heart disease and stroke statistics—2012 update: A report from the American Heart Association” Circulation 2012; 125:188-197
4. Levy et al. “Long-term trends in the incidence of and survival with heart failure” The New England Journal of Medicine 2002; 347:1397-1402
5. Damasceno et al. “Heart failure in sub-saharan Africa: Time for action” Journal of the American College of Cardiology 2007; 50:1688-1693
6. Jessup et al. “Calcium upregulation by percutaneous administration of gene therapy in cardiac disease (cupid): A phase 2 trial of intracoronary gene therapy of sarcoplasmic reticulum Ca2+-ATPase in patients with advanced heart failure” Circulation 2011; 124:304-313
7. Hare et al. “Comparison of allogeneic vs autologous bone marrow-derived mesenchymal stem cells delivered by transendocardial injection in patients with ischemic cardiomyopathy: The POSEIDON randomized trial” JAMA 2012; 308:2369-2379
8. Piacentino et al. “X-linked inhibitor of apoptosis protein-mediated attenuation of apoptosis, using a novel cardiac-enhanced adeno-associated viral vector” Hum Gene Ther 2012; 23:635-646
9. Kline et al. “High quality catalog of proteotypic peptides from human heart” J Proteome Res 2008; 7:5055-5061
10. Soderblom et al. “Quantitative label-free phosphoproteomics strategy for multifaceted experimental designs” Anal Chem 2011; 83:3758-3764
11. Weng et al. “Rosetta error model for gene expression analysis” Bioinformatics 2006; 22:1111-1121
12. Cyr et al. “Characterization of serum proteins associated with i128b genotype among patients with chronic hepatitis C” PLoS One 2011; 6:e21854
13. Patel et al. “High predictive accuracy of an unbiased proteomic profile for sustained virologic response in chronic hepatitis C patients” Hepatology 2011; 53:1809-1818
14. Reidel et al. “Proteomic profiling of a layered tissue reveals unique glycolytic specializations of photoreceptor cells” Mol Cell Proteomics 2011; 10:M110 002469
15. Saka et al. Quantitative proteomics reveals metabolic and pathogenic properties of Chlamydia trachomatis developmental forms” Mol Microbiol 2011; 82:1185-1203
16. Geromanos et al. The detection, correlation, and comparison of peptide precursor and product ions from data independent lc-ms with data dependant lc-ms/ms. Proteomics. 2009; 9:1683-1695
17. Li et al. Database searching and accounting of multiplexed precursor and product ion spectra from the data independent analysis of simple and complex peptide mixtures. Proteomics. 2009; 9:1696-1719
18. Keller et al. Empirical statistical model to estimate the accuracy of peptide identifications made by ms/ms and database search. Anal Chem. 2002; 74:5383-5392
19. Nesvizhskii et al. A statistical model for identifying proteins by tandem mass spectrometry. Anal Chem. 2003; 75:4646-4658
20. Jourdan-Lesaux et al. Extracellular matrix roles during cardiac repair. Life sciences. 2010; 87:391-400
21. Vilahur et al. Reperfusion-triggered stress protein response in the myocardium is blocked by post-conditioning. Systems biology pathway analysis highlights the key role of the canonical aryl-hydrocarbon receptor pathway. European heart journal. 2012. In press.
22. Chaanine et al. Akt signalling in the failing heart. Eur J Heart Fail. 2011; 13:825-829
24. Wei et al. Proteomic analysis reveals significant elevation of heat shock protein 70 in patients with chronic heart failure due to arrhythmogenic right ventricular cardiomyopathy. Mol Cell Biochem. 2009; 332:103-111
25. Basar et al. Lower fetuin-a predicts angiographic impaired reperfusion and mortality in st-elevation myocardial infarction. Journal of investigative medicine: the official publication of the American Federation for Clinical Research. 2011; 59:816-822
26. Rosello-Lleti et al. Cardiac protein changes in ischaemic and dilated cardiomyopathy: A proteomic study of human left ventricular tissue. J Cell Mol Med. 2012; 16:2471-2486
27. Anderson et al. The human plasma proteome: History, character, and diagnostic prospects. Mol Cell Proteomics. 2002; 1:845-867
28. States et al. Challenges in deriving high-confidence protein identifications from data gathered by a hupo plasma proteome collaborative study. Nat Biotechnol. 2006; 24:333-338
29. Frustaci et al. Celiac disease associated with autoimmune myocarditis. Circulation. 2002; 105:2611-2618
30. Shukla et al. Does oxidative stress change ceruloplasmin from a protective to a vasculopathic factor? Atherosclerosis. 2006; 187:238-250
31. Padmini et al. Heat-shock protein 90 alpha (hsp90alpha) modulates signaling pathways towards tolerance of oxidative stress and enhanced survival of hepatocytes of mugil cephalus. Cell Stress Chaperones. 2011; 16:411-425
32. Johnson et al. Serum amyloid a as a predictor of coronary artery disease and cardiovascular outcome in women: The national heart, lung, and blood institute-sponsored women's ischemia syndrome evaluation (wise). Circulation. 2004; 109:726-732
33. Hua et al. A role for acute-phase serum amyloid a and high-density lipoprotein in oxidative stress, endothelial dysfunction and atherosclerosis. Redox Rep. 2009; 14:187-196
34. Sottrup-Jensen L. Alpha-macroglobulins: Structure, shape, and mechanism of proteinase complex formation. J Biol Chem. 1989; 264:11539-11542
35. French et al. Protease activation of alpha2-macroglobulin modulates a chaperone-like action with broad specificity. Biochemistry. 2008; 47:1176-1185
36. Neubauer S. The failing heart—an engine out of fuel. N Engl J Med. 2007; 356:1140-1151
37. Lopaschuk et al. Myocardial fatty acid metabolism in health and disease. Physiol Rev. 2010; 90:207-258
38. Lionetti et al. Modulating fatty acid oxidation in heart failure. Cardiovasc Res. 2011; 90:202-209
39. Ussher et al. Stimulation of glucose oxidation protects against acute myocardial infarction and reperfusion injury. Cardiovasc Res. 2012; 94:359-369
47. Barallobre-et al. Proteomics analysis of cardiac extracellular matrix remodeling in a porcine model of ischemia/reperfusion injury. Circulation. 2012; 125:789-802
48. Argraves et al. Fibulins: Physiological and disease perspectives. EMBO reports. 2003; 4:1127-1131
49. Nakatani et al. Targeted proteomics of isolated glomeruli from the kidneys of diabetic rats: Sorbin and sh3 domain containing 2 is a novel protein associated with diabetic nephropathy. Experimental diabetes research. 2011; 2011:979354
50. Sanger et al. Arg/abl-binding protein, a z-body and z-band protein, binds sarcomeric, costameric, and signaling molecules. Cytoskeleton. 2010; 67:808-823.

### Example 2

### Phosphoproteomic Profiling of Human Myocardial Tissues Distinguishes Ischemic from Non-Ischemic End Stage Heart Failure

Background:

To develop more effective heart failure therapeutics, the present study was conducted to better understand the molecular differences between ischemic (IF) and non-ischemic (NIF) heart failure. In this study, extensive proteomic and phosphoproteomic profiles of myocardial tissue from patients diagnosed with IF or NIF were assembled and compared.

Methods and Results:

Proteins extracted from left ventricular sections were proteolyzed, and phosphopeptides were enriched using titanium dioxide resin. Gel- and label-free nanoscale capillary liquid chromatography coupled to high-resolution, accuracy mass tandem mass spectrometry allowed for the quantification of 4,436 peptides (corresponding to 450 proteins) and 823 phosphopeptides (corresponding to 400 proteins) from the unenriched and phospho-enriched fractions, respectively. There were no proteins exhibiting a significant difference in abundance between the NIF and IF samples, with the exception of carbonic anhydrase 3. In contrast, 37 peptides (corresponding to 26 proteins) exhibited a ≧2-fold alteration in phosphorylation state (p<0.05) when comparing IF and NIF. The degree of protein phosphorylation at these 37 sites was specifically dependent upon the heart failure etiology examined. Proteins exhibiting phosphorylation alterations were grouped into functional categories: transcriptional activation/RNA processing; cytoskeleton structure/function; molecular chaperones; cell adhesion/signaling; apoptosis; and energetic/metabolism.

Conclusions:

Phosphoproteomic analysis demonstrated profound post-translational differences in proteins that are involved in multiple cellular processes between different heart failure phenotypes. Understanding the roles these phosphorylation alterations play in the development of NIF and IF has the potential to generate etiology-specific heart failure therapeutics, which could be more effective than current therapeutics in addressing the growing concern of heart failure.

Introduction:

Despite improved therapy and earlier diagnosis, heart failure (HF) continues to be a major health concern, with 5.7 million Americans diagnosed with HF in 2012.¹ The lifetime risk of developing HF after age 40 is 20%, with the annual incidence approaching 10 per 1000 people after age 65.² More than half of all HF patients will die within a 5-year period of being diagnosed.^{3,4} These statistics are complicated by the fact that HF is a complex, multi-faceted disease that presents in two major forms.

The two distinct types of heart disease that can lead to HF are ischemic and non-ischemic cardiomyopathy. Ischemic HF describes significantly impaired left ventricular function resulting from reduced blood supply to the heart muscle, most commonly from coronary artery disease. In contrast, non-ischemic HF has a range of etiologies, including congenital, infectious agents, autoimmune, and idiopathic. Regardless of etiology, current standard of care treats HF similarly. The treatment options for advanced heart failure are limited to implantation of a ventricular assist device to mechanically unload the heart, heart transplantation, or palliation with continuous intravenous inotropic support. These options, however, are also associated with high morbidity and mortality, highlighting the continued need for HF therapeutic development.

One such avenue of exploration is etiology-specific treatment. Such precise therapy requires an enhanced understanding of the molecular differences between the different heart failure phenotypes. Uncovering the molecular differences in a more systematic and comprehensive way is made possible by utilizing high throughput 'omics profiling^{5,6}

In the current investigation, cardiac tissue from a well-characterized human heart tissue bank was subjected to titanium dioxide resin to enrich for phosphopeptides, which were then analyzed by a bottoms-up LC/MS/MS global proteomics approach. This approach revealed amino acid residue-specific phosphorylation patterns on 400 cardiac proteins, which were compared between the IF and NIF etiologies. This revealed, for the first time, cardiac disease-specific phosphorylation pattern variations on key proteins involved in various aspects of cardiac physiology. Understanding how distinct protein phosphorylation patterns impact specific heart failure etiologies will support the development of therapeutics that better treat heart failure.

### Human Cardiac Tissue Acquisition and Tissue Repository

Human myocardium was acquired from the left ventricular (LV) free wall of explanted ischemic failing (IF) or non-ischemic failing (NIF) hearts following cardiac transplantation. Non-failing (NF) left ventricular tissue was acquired from donors whose hearts were suitable for transplantation but for a variety of reasons, were not utilized for transplant and became available for research. After explantation, transmural tissue samples were processed and stored as described in supplemental methods.

### Sample Preparation for Mass Spectrometry

Heart tissue samples were homogenized and subjected to phase separation by addition of chloroform. Protein was precipitated from the organic layer, washed, sonicated, recovered by centrifugation, and re-suspended in mass spectrometry-compatible detergent (RapiGest, Waters Corp., Milford, Mass.). A 625 μg aliquot of protein (per sample) was subjected to reduction, and alkylation, followed by overnight proteolysis with sequencing grade trypsin (Promega, Madison, Wis.). A 25 μg aliquot from each sample was used for unenriched proteomic analysis of protein expression in the heart tissue. This 25 μg was spiked with 1.25 pmol ADH1_YEAST digest (Massprep standard, Waters Corporation) as a surrogate standard prior to analysis. The remaining 600 μg of protein was then enriched for phosphopeptides using in-house packed TiO₂ spin columns as previously described.⁹

### LC/MS/MS Data Collection

The sample cohort was randomized prior to LC/MS/MS analysis. Peptide digests obtained from each of the samples were analyzed in a label-free quantitative fashion using a nanoAcquity UPLC system coupled to a Synapt HDMS mass spectrometer (Waters Corp, Milford, Mass.) for unenriched peptide analyses and an LTQ Orbitrap XL (Thermo Fisher Scientific, Waltham, Mass.) for phosphopeptide analyses.

### LC-MS Data Processing

Robust peak detection and label-free alignment of individual peptides across all sample injections was performed using the commercial package Rosetta Elucidator® software, v3.3 (Rosetta Biosoftware, Inc., Seattle, Wash.) with PeakTeller algorithm.¹⁰

### Statistical Analysis

Basic statistical analysis was performed on both the unenriched (protein-level) and phosphopeptide (peptide-level) datasets in order to obtain candidate (phospho) proteins that were differentially expressed. Fold-changes were calculated for each failing group versus non-failing control, as the ratio of the average intensity between the groups; directionality of the ratio was established that positive fold-changes mean up-regulated in failing versus nonfailing control, and negative fold-changes mean down-regulated in failing versus nonfailing. P-values were calculated using an error-weighted ANOVA with Benjamini-Hochberg FDR correction for multiple hypotheses testing (Rosetta Elucidator® software, v3.3). The input for this test was the protein-level data for unenriched analysis (intensity for all peptides summed per sample), or the peptide-level data for phosphopeptides, and the raw intensities were scaled to a normal distribution using the Error Model in Elucidator software prior to ANOVA. Fold-changes and p-values are shown for all proteins (Table 7) and phosphopeptides (Table 8). Statistical cutoffs for fold-change were established for proteins based on a power calculation using the average biological variation within each group. Using the protein average % CV (23%) and 4 reps per group at a 95% confidence, minimum cutoffs were set to 2-fold (98% powering) for proteins. ANOVA p-value of 0.05 or less was required. The differentially expressed proteins meeting these criteria are shown in Table 2.

### Western Blot Analysis

Cryopreserved heart tissue was mechanically disrupted by mortar and pestle in liquid nitrogen and suspended in lysis buffer (1% IGEPAL CA-630, 0.5% Deoxycholate, 2% SDS, 5 mM EDTA in 1×PBS) with protease and phosphatase inhibitor cocktail tablets (Roche Diagnostics, Indianapolis, Ind.). Samples were then pulse homogenized on ice with a handheld tissue tearor (BioSPEC Products Inc., Model 985-370).

Cardiac tissue homogenates were subjected to Bicinchoninic acid (BCA) assay (Pierce Biotechnology/Thermo Fisher Scientific, Rockford, Ill.) for protein quantification. Western Blot for protein immunodetection was performed using a modification of a recently published method.¹¹

### Metabolomics

Organic acids were quantified using methods described previously¹² employing Trace Ultra GC coupled to ISQ MS operating under Xcalibur 2.2 (Thermo Fisher Scientific, Austin, Tex.).

### Enzymatic Activity Assays

LDH activity was assayed using the CytoTox-ONE Homogenous Membrane Integrity Assay in a GloMax-Multi+ Microplate reader, both by Promega (Madison, Wis.) following the manufacturer's instructions.

PDH activity was assessed using the PDH Enzyme Activity Microplate Assay Kit from abcam (ab109902, Cambridge, Mass.). Briefly, ˜40 mg of heart tissue was homogenized in PBS containing protease and phosphatase inhibitors and apyrase. Homogenates were further solubilized using the manufacturer's detergent. Then, homogenates were centrifuged at 3,500 rpm at 4° C. Protein concentration of the lysates was examined using a BCA assay (Pierce), and 100 ug of total protein was added to the assay kit 96-well plate. Following a 3-hr incubation at room temperature, the wells were washed, assay solution was added, and PDH activity was read at 450 nm on a plate reader for 20 min at 20 sec intervals.

### Bioinformatics Analysis of Protein Data Sets

Ingenuity Pathway Analysis (IPA, Winter 2012 Release) (Ingenuity Systems, Redwood City, Calif.) was used to classify the proteins according to primary function as well as for pathway analysis. KinasePhose 2.0 (kinasephos2.mbc.nctu.edu.tw/index.html)¹³ was used to identify protein-kinase specific phosphorylation sites among the differentially phosphorylated proteins with a specificity threshold of at least 80%.

### Patient Samples

Left ventricular tissue from 12 male patients matched for age and race was used in this study (Table 1). The tissue was acquired from three groups (n=4 per group): explanted hearts from transplant patients with either ischemic cardiomyopathy (ischemic failing, IF) or non-ischemic cardiomyopathy (non-ischemic failing, NIF), or non-failing (NF) donor hearts not used for transplantation. Groups were closely matched for cardiac function and treatment history (Table 1). All patients with HF exhibited significantly lower ejection fractions (EF) compared to NF controls, which had normal left ventricular function. All HF patients received intravenous inotropic agents and intra-aortic balloon pump support, whereas only patients from the IF subset received prior coronary artery bypass surgery. In the NIF group, two patients were diagnosed with non-ischemic cardiomyopathy of unknown etiology, one patient had a viral cardiomyopathy, and the fourth patient developed HF secondary to valvular disease.

### Quantitative Analysis of Proteins in Human Left Ventricles

Following the workflow in FIG. 1A and as outlined herein, all heart tissue samples were subjected to quantitative analyses of the phospho-enriched and unenriched proteome. The reproducibility of the unenriched analytical approach was validated with a spike-in of the internal standard yeast ADH1 digest at a known concentration. Consistent ADH1 abundance (7% coefficient of variation (CV)) across all 12 samples was observed (FIG. 1B).

Peptide/protein identification and quantification by Rosetta Elucidator® software with Mascot and IdentityE search algorithms yielded expression data for a total of 850 proteins: 4,436 peptide annotations representing 450 proteins in the unenriched samples, for which relative quantitation between samples was performed (FIG. 1C; Table 7), and site-specific quantitation for 823 phosphopeptides corresponding to 400 phosphorylated proteins in the phospho-enriched samples were determined (FIG. 1C; Table 8). Between the observed unenriched proteome and the phosphoproteome, 68 proteins overlapped (FIG. 1C). These 68 proteins and their respective fold changes and p-values are listed in Tables 9 and 10.

### Differential Expression Analysis

Principal components analysis (PCA) was performed in order to observe any high-level differences between sample groups and to screen for outlier samples using the unenriched and phospho-enriched proteome expression data. Expression data was z-score transformed across all samples at the protein-level for unenriched samples (FIG. 2A) or at the peptide level for phospho-enriched samples (FIG. 2B), and Rosetta Elucidator® software, v3.3 was used to calculate principal components. The two most prominent components (PC1 versus PC2) are plotted in FIGS. 2A and 2B, and no significant outliers are observed for either unenriched or phosphoproteomes. Interestingly, in unenriched proteomes (FIG. 2A), the four non-failing (NF) samples separate from the failing samples along PC1; however, in the phosphoproteome, at least three of the four non-ischemic failing (NIF) appear unique along PC1 compared to the IF or NF groups (FIG. 2B). This global observation of group classification is at least suggestive that the unenriched proteome may best separate failing from nonfailing hearts, whereas phosphorylation status may play a larger role in distinguishing ischemic versus non-ischemic failure.

Using the levels of significantly differentially abundant proteins and phosphopeptides, an unbiased 2D hierarchical clustering analysis was performed at the protein-level (FIG. 2C) or phosphopeptide level (FIG. 2D) in order to observe the overall expression pattern of these molecules in each individual sample and to determine how these candidates seem to segregate the failing and nonfailing hearts. FIG. 2C shows clear differentiation between failing and nonfailing hearts but no clear differentiation between the two failing etiologies using proteins from the unenriched analyses. In contrast to the unenriched analyses, the phosphopeptides (FIG. 2D) show a potentially more robust differentiation between all three groups.

Comparison of the unenriched NIF and IF proteomes revealed no differential protein expression except for carbonic anhydrase 3; however, comparison of the unenriched failing and NF hearts uncovered 31 distinct proteins, which were represented by at least two high-confidence peptides and a significant (ANOVA, p<0.05) fold change of at least two (Tables 2A and B). Of this group, 23 proteins in NIF and eight in IF differed in abundance from the NF. Moreover, four proteins were decreased in abundance, while the remaining proteins were increased in abundance in NIF and IF hearts (Tables 2A and B). Among the proteins significantly changed in NIF and IF human myocardial tissue were secreted glycoproteins, ceruloplasmin, carbonic anhydrase, serum amyloid A, and extracellular matrix proteins. Peptides from the fibulin family of extracellular matrix proteins (fibulin 1, fibulin2, and latent transforming growth factor beta binding protein 2 (fibulin 3)) were consistently up-regulated across the NIF samples.

The primary goal was to determine etiology-specific changes in site-specific phosphorylation for the phosphopeptides. A phosphopeptide was considered of interest if it fulfilled the following criteria: 1) a ≧2-fold alteration in phosphorylation state (ANOVA p-value ≦0.05) between NIF and IF; 2) significantly different phosphorylation levels between at least one etiology of failing hearts and NF hearts; and 3) unlikely to be due to protein level differences. The last criterion was fulfilled by using either the unenriched protein levels (if available; Tables 9 and 10) or the comparative levels of other phosphopeptides from the protein of interest and demonstrating that the levels of these other phosphopeptides are not significantly different between the three heart tissue groups nor do they trend in the same direction as the phosphopeptide under consideration.

From this analysis, 26 proteins were identified to contain at least one differently phosphorylated site between NIF and IF tissue that met our criteria (Table 5). Some of these proteins, such as lyric and leiomodin-1, were differentially phosphorylated, more particularly at serine 298 of lyric and at serines 508, 512, 516, 520 and 555 of Leimodin-1, in the two types of heart failure.

LYRIC (Uniprot Accession No. Q86UE4)

| | |
|---|---|
| 1 | MAARSWQDEL AQQAEEGSAR LREMLSVGLG |
| | FLRTELGLDL GLEPKRYPGW VILVGTGALG |
| 61 | LLLLFLLGYG WAAACAGARK KRRSPPRKRE |
| | EAAAVPAAAP DDLALLKNLR SEEQKKKNRK |
| 121 | KLSEKPKPNG RTVEVAEGEA VRTPQSVTAK |
| | QPPEIDKKNE KSKKNKKKSK SDAKAVQNSS |
| 181 | RHDGKEVDEG AWETKISHRE KRQQRKRDKV |
| | LTDSGSLDST IPGIENTITV TTEQLTTASF |
| 241 | PVGSKKNKGD SHLNVQVSNF KSGKGDSTLQ |
| | VSSGLNENLT VNGGGWNEKS VKLSSQISAG |
| 301 | EEKWNSVSPA SAGKRKTEPS AWSQDTGDAN |
| | TNGKDWGRSW SDRSIFSGIG STAEPVSQST |
| 361 | TSDYQWDVSR NQPYIDDEWS GLNGLSSADP |
| | NSDWNAPAEE WGNWVDEERA SLLKSQEPIP |
| 421 | DDQKVSDDDK EKGEGALPTG KSKKKKKKKK |
| | KQGEDNSTAQ DTEELEKEIR EDLPVNTSKT |
| 481 | RPKQEKAFSL KTISTSDPAE VLVKNSQPIK |
| | TLPPATSTEP SVILSKSDSD KSSSQVPPIL |
| 541 | QETDKSKSNT KQNSVPPSQT KSETSWESPK |
| | QIKKKKKARR ET |

Leiomodin-1 (Uniprot Accession No. P29536)

| | |
|---|---|
| 1 | MSRVAKYRRQ VSEDPDIDSL LETLSPEEME |
| | ELEKELDVVD PDGSVPVGLR QRNQTEKQST |
| 61 | GVYNREAMLN FCEKETKKLM QREMSMDESK |
| | QVETKTDAKN GEERGRDASK KALGPRRDSD |
| 121 | LGKEPKRGGL KKSFSRDRDE AGGKSGEKPK |
| | EEKIIRGIDK GRVRAAVDKK EAGKDGRGEE |
| 181 | RAVATKKEEE KKGSDRNTGL SRDKDKKREE |
| | MKEVAKKEDD EKVKGERRNT DTRKEGEKMK |
| 241 | RAGGNTDMKK EDEKVKRGTG NTDTKKDDEK |
| | VKKNEPLHEK EAKDDSKTKT PEKQTPSGPT |
| 301 | KPSEGPAKVE EEAAPSIFDE PLERVKNNDP |
| | EMTEVNVNNS DCITNEILVR FTEALEFNTV |
| 361 | VKLFALANTR ADDHVAFAIA IMLKANKTIT |
| | SLNLDSNHIT GKGILAIFRA LLQNNTLTEL |
| 421 | RFHNQRHICG GKTEMEIAKL LKENTTLLKL |
| | GYHFELAGPR MTVTNLLSRN MDKQRQKRLQ |
| 481 | EQRQAQEAKG EKKDLLEVPK AGAVAKGSPK |
| | PSPQPSPKPS PKNSPKKGGA PAAPPPPPPP |
| 541 | LAPPLIMENL KNSLSPATQR KMGDKVLPAQ |
| | EKNSRDQLLA AIRSSNLKQL KKVEVPKLLQ |

Other phosphoproteins demonstrated either a significant hyper-phosphorylation or dephosphorylation in one or more amino acid sites while phosphorylation levels at the corresponding amino acid sites were unchanged in the other etiology compared to NF tissue. Despite the variety of proteins listed, common functional themes of the significant phosphoproteins include cell growth/death, cardiac muscle development and/or function, and stress response, suggesting etiology-specific regulation of these functions contribute to the development or progression of heart failure.

### Validation Western Blot Analysis

The unenriched proteomic mass spec data were validated from independently prepared tissue homogenates from the same 12 patients by Western blot (FIG. 4A). Western blot quantitative data is shown in FIG. 4B. These Western blots confirmed the altered changes in IF and NIF compared to NF that were revealed by the mass spec data: an increase in carbonic anhydrase, ceruloplasmin, Fibulin1, Fibulin2 and Alpha 2-HS glycoprotein (FETUA); a decrease in serum amyloid A; and an increase in α2-Macroglobulin (α2M). Interestingly, a cleaved version of α2M was the prevalent form of α2M in both NIF and IF hearts, whereas the full-length α2M was the prevalent species in NF heart tissue (FIGS. 4A and 4B).

### Metabolomics Analyses

A decrease in phosphorylation of pyruvate dehydrogenase E1 component subunit α (ODPA, NCBI Reference Sequence No. NP_{—}000275.1) at Ser232, 293, 300 was observed in IF heart compared to both NIF and NF samples (Table 5), indicating a significant increase in ODPA activity.

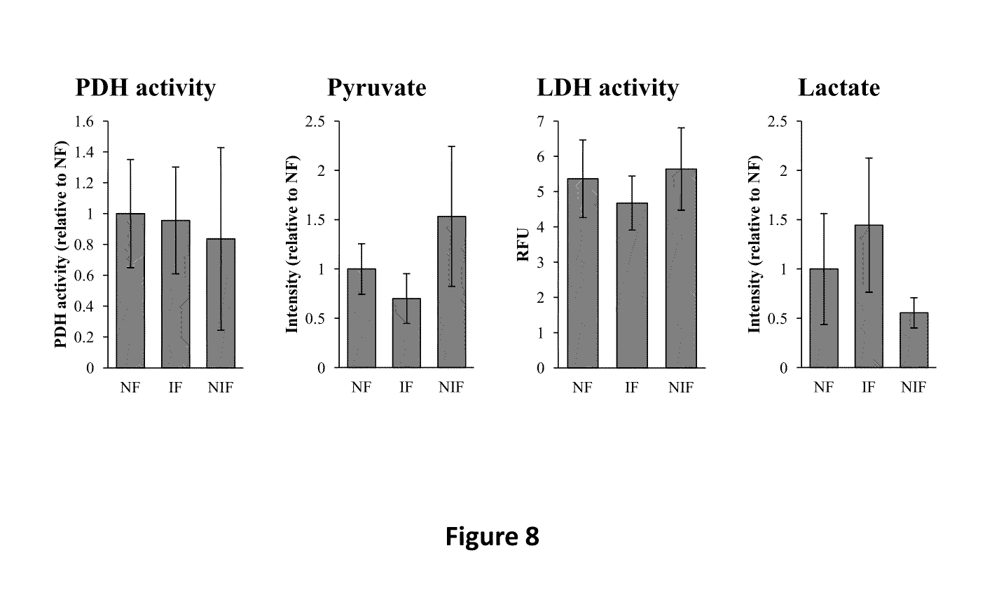

ODPA catalyzes the overall conversion of pyruvate to acetyl-CoA and CO₂, and provides the primary link between glycolysis and the tricarboxylic acid cycle. Therefore, altered ODPA phosphorylation can contribute to marked metabolic consequences. To explore the metabolic consequences of ODPA differential phosphorylation in the same 12 human myocardial samples, mass spectrometry methods were used to examine pyruvate content. Although pyruvate levels were not significantly different in either NIF or IF compared to NF, they were suggestive of differentiating etiology (p=0.06) (FIG. 8). Besides decreased ODPA activity, the pyruvate pool can increase by increased lactate dehydrogenase (LDH) activity in the presence of high amounts of lactate. Therefore, in the same human tissue samples, we also assessed both lactate levels and LDH activity (FIG. 8). As with pyruvate, lactate levels were different only between etiologies (p=0.04); lactate was higher in IF and lower in NIF. However, LDH activity was not different between groups (p=0.426). Finally, in an effort to affirm the phospho-PDH data, a PDH enzyme assay was employed but results indicated that PDH activity did not differ between groups (p=0.866).

### Ingenuity Analysis

Focusing on differential protein or phosphoprotein levels between NIF and NF samples, Ingenuity Pathway Analysis (IPA) was used to construct an interaction network of proteins (FIG. 5). Many of the proteins that were significantly different (>2-fold change, p<0.05) were glycoproteins, proteoglycans, and structural proteins, all components of the extracellular matrix (ECM). More ECM proteins were discovered when the inclusion criteria were expanded to significantly different proteins between NIF and NF with <2-fold change (min fold change 1.46) using the IPA analysis (FIG. 5). Pathway analysis also suggested the possibility that Akt, SMAD3, MMP14, and aryl hydrocarbon receptor (AHR), all of which have been implicated in cardiac remodeling,¹⁴⁻¹⁷ may be central regulators controlling the expression levels of many of the proteins included in the IPA analysis. IPA analysis validation was performed by Western blot analysis, which established that several of these more central proteins (AKT, SMAD3, and AHR) were also differentially expressed in NIF (FIGS. 6A and B).

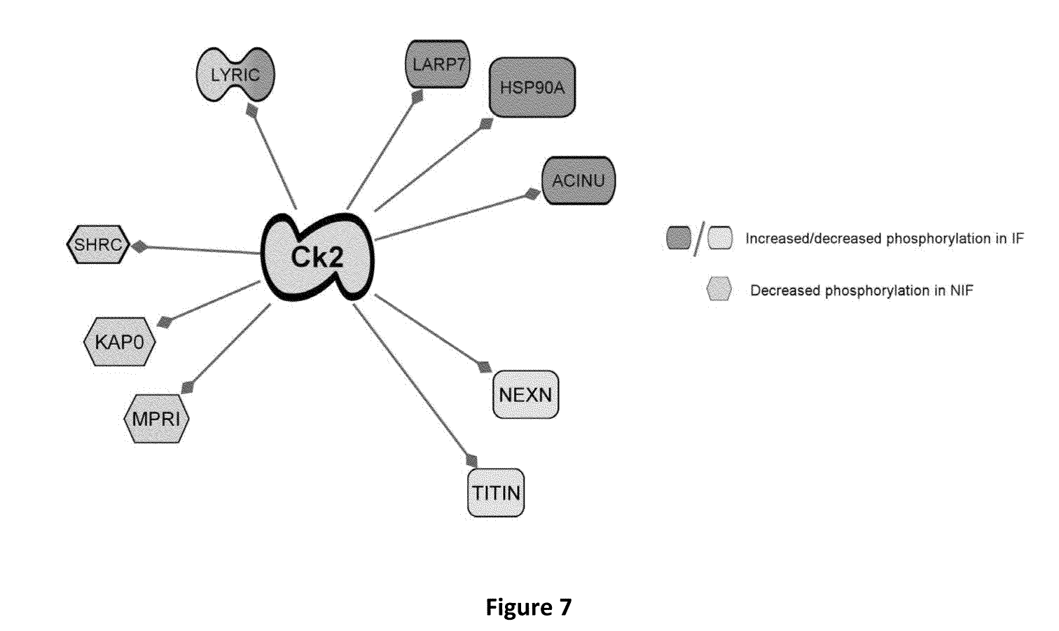

Further bioinformatics analysis of the phosphorylation data demonstrated that nine of the differentially phosphorylated proteins are possible targets of casein kinase 2 (Ck2, FIG. 7). The four differentially phosphorylated proteins in NIF (LYRIC, SHRC, FHL2, KAP0 and MPRI) were all dephosphorylated in this disease state, while four of the six differentially phosphorylated proteins in IF (LYRIC, LARP7, HSP90A, ACINU) were increasingly phosphorylated.

Analysis of all significant unenriched proteins (p<0.05), regardless of fold change, showed that the following canonical pathways were significantly associated with both NIF and IF: mitochondrial dysfunction, calcium signaling, and acute phase response signaling (Table 6).

Heart failure remains a progressive disease with limited treatment options. Current standard of care does not differentiate between ischemic and non-ischemic heart failure, treating them similarly. However, as this study indicates, there are important post-translational phosphorylation differences in proteins involved in a variety of cellular processes between the two heart failure etiologies. These differences may be critical in the design of therapies for heart failure.

This analysis revealed the following general differences between the two main types of heart failure: 1) With one exception, protein abundances were similar between both types of heart failure compared to NF controls; and 2) For many proteins, site-specific phosphorylation differences clearly distinguished NIF and IF.

### Protein Abundance Differences Between Non-Ischemic and Ischemic Heart Tissue

The only protein that displayed statistically significant differential expression between NIF and IF was carbonic anydrase 3 (CAH3). CAH3 levels were 2.83-fold higher in the NIF than in the IF samples. Why the levels of CAH3 increase in HF, especially in NIF, is unclear even though CAH3 is highly expressed in heart tissue. In general, many tissues contain carbonic anhydrases, which interconvert carbon dioxide and bicarbonate to maintain acid-base balance and help transport carbon dioxide out of tissues, especially metabolically active tissues like heart and brain. The use of carbonic anhydrase inhibitors (e.g., acetazolamide and zonisamide) in the treatment of HF should be explored. Alternatively, up-regulation of CAH3 may be a positively adaptive change, modulating CAH3 levels by up-regulation of CAH3 levels and/or activity may provide a therapeutic benefit.

### Protein Abundance Differences Between Failing and Non-Failing Human Tissue

Extracellular Matrix Remodeling Especially in the Context of NIF

The involvement of ECM remodeling in NIF was the most robust finding from evaluation of protein level differences (FIG. 5). This finding complements and adds to the results from other studies that have used proteomic analysis to investigate protein expression variation in cardiac disease^{18,19} TGFB1 was heavily involved in this network (FIG. 5). Additionally, this network revealed that fibulin 1, 2, and 3 (components of elastic fibers) were significantly upregulated in NIF and trended toward significant upregulation in IF. These revelations were confirmed by Western blot analysis of fibulin 1 and 2 (FIG. 4). Development of strategies to downregulate the levels of fibulins may be therapeutic for heart failure.

Not only are TGFB1 and fibulins independently implicated in heart failure, they may function in concert. TGFB1 signaling is predicted to control fibulin 1 and 2 levels as seen in this interaction network. Additionally, pathway analysis revealed other central players controlling fibulin levels, including SMAD3 and AHR—two transcriptional factors involved in the TGFB1 signaling cascade and regulation of biological responses to planar aromatic hydrocarbons, respectively. Both AHR and SMAD3 levels were demonstrated to be modulated in failing human heart tissue, with SMAD3 significantly upregulated in NIF (FIG. 6). Therefore, one way to modulate fibulin levels in the failing heart may be through SMAD3 and/or AHR.

Oxidative Stress, Cellular Damage, and Inflammation

In the oxidative stress and inflammatory pathways, NIF and IF samples had similarly modified proteins, including ceruloplasmin, heat shock protein 90, serum amyloid A, and α2-macroglobulin (α2M). These proteins had comparative abundances in human heart tissue that were confirmed by Western blot analyses (FIG. 4).

The increased expression of both ceruloplasmin and HSP90 suggests that constant cellular stress and inflammation underlies the development of HF.

When exposed to a protease, α2M exposes a “bait region” within its structure that traps the protease. Following protease binding, α2M promotes clearance and degradation of the bound protease while undergoing limited proteolysis.²⁶ This limited proteolysis, however, does result in α2M cleavage, and these findings reveal an increase in α2M cleavage products. Therefore, the increased levels of cleaved α2M in HF tissue could be protective attempts to limit the effects of harmful proteases. In addition to binding proteases, the native form of α2M can bind to damaged proteins in order to prevent cellular damage and abnormal protein deposition. This broader protective function of α2M is lost, however, upon exposure to a protease.²⁷. Moreover, the decreased ability of α2M to bind to and clear damaging protein deposits could be contributing to the progression of HF. Whether a cause of or response to cellular damage, the cleaved form of α-2M could be developed into a biomarker for the progression of HF.

### Phosphorylation Reveals Key Difference Between NIF and IF Etiologies

While the unenriched data set demonstrates the common pathways underlying both types of heart failure, the phosphorylation data begins to elucidate differences between the two disease etiologies. Notably, there were two types of phosphorylation differences: 1) etiology-specific differences in phosphorylation at a particular site; and 2) significantly different single- or multiple-site phosphorylation in only one type of HF (compared to NF control). Another notable finding was that over one-third of the differentially phosphorylated protein sequences (9/26) were potential targets of casein kinase 2 (Ck2). (FIG. 7).

Etiology-Specific Differences in Phosphorylation

Lyric, also known as metadherin (Uniprot Accession No. Q86UE4), is a transcription co-factor that has recently been found to play a major role in cancer cell survival.²⁸ Survival is associated with increased Lyric content and phosphorylation.^{28,29} Phosphorylation of Lyric has not been associated in heart failure. The NIF heart samples in this study displayed increased phosphorylation at Lyric amino acid 298, whereas IF samples displayed a decrease in lyric phosphorylation at the same site (Table 5).

Although no studies have established the role of phosphorylation at site 298 in the regulation of Lyric activity, other regions on the Lyric protein are involved in protein/protein interactions and appear to be regulated by phosphorylation events. Assuming that this phosphorylation/desphosphorylation of Lyric at 298 also functions as a toggle switch for protein binding, the increased phosphorylation in IF could be an attempt to recruit a partner transcription factor to activate cell growth in the viable myocardium to compensate for the injury already suffered. Conversely, dephosphorylation of the pro-growth Lyric protein could be contributing to the activation of pro-cell death pathways or suppression of pro-cell growth pathways within the NIF heart.

Heart failure etiology may also be separated based on amount of leiomodin-1 phosphorylation (Table 5). Multiple amino acid sites on leiomodin-1 exhibited greater phosphorylation in the NIF samples than the IF samples, with fold differences ranging from 4 to 9 fold greater, depending on the site. Generation of mutants in leiomodin-1 unable to be phosphorylated at the amino acids revealed herein will allow for a determination of the involvement of this relatively uncharacterized protein in the development of these two distinct types of heart failure.

Significantly Different Phosphorylation Only Seen in One HF Etiology

In this study, phosphorylation of basigin at serine 368 near the C-terminus was increased in IF (compared to NIF and NF).

Basigin (NCBI Reference Sequence No. NP_001719.2)

| | |
|---|---|
| 1 | MAAALFVLLG FALLGTHGAS GAAGFVQAPL SQQRWVGGSV |
| | ELHCEAVGSP VPEIQWWFEG |
| 61 | QGPNDTCSQL WDGARLDRVH IHATYHQHAA STISIDTLVE |
| | EDTGTYECRA SNDPDRNHLT |
| 121 | RAPRVKWVRA QAVVLVLEPG TVFTTVEDLG SKILLTCSLN |
| | DSATEVTGHR WLKGGVVLKE |
| 181 | DALPGQKTEF KVDSDDQWGE YSCVFLPEPM GTANIQLHGP |
| | PRVKAVKSSE HINEGETAML |
| 241 | VCKSESVPPV TDWAWYKITD SEDKALMNGS ESRFFVSSSQ |
| | GRSELHIENL NMEADPGQYR |
| 301 | CNGTSSKGSD QAIITLRVRS HLAALWPFLG IVAEVLVLVT |
| | IIFIYEKRRK PEDVLDDDDA |
| 361 | GSAPLKSSGQ HQNDKGKNVR QRNSS |

POPD1, a member of the Popeye domain containing family, is a membrane protein abundantly expressed in the heart. While the overall abundance of POPD1 was not changed between the three groups in these data, POPD1 phosphorylation at 2 sites was diminished in NIF heart relative to NF heart, while phosphorylation at these sites was minimally changed in IF (Table 5). Interestingly, in this study, slow cardiac myosin regulatory light chain 2 (MLRV) was significantly less phosphorylated at serine 15 in NIF compared to IF heart (Table 5).

| MLRV (GenBank Accession No. AAH31006.1) | |
|---|---|
| 1 | MAPKKAKKRA GGANSNVFSM FEQTQIQEFK |
| | EAFTIMDQNR DGFIDKNDLR DTFAALGRVN |
| 61 | VKNEEIDEMI KEAPGPINFT VFLTMFGEKL |
| | KGADPEETIL NAFKVFDPEG KGVLKADYVR |
| 121 | EMLTTQAERF SKEEVDQMFA AFPPDVTGNL |
| | DYKNLVHIIT HGEEKD |

Together, these findings further suggest additional etiology-specific phosphorylation events in the RhoA/POPD1/MLRV pathway play a role in the development of advanced heart failure.

Multiple sites on SORBS2 exhibited increased phosphorylation in this study in NIF samples compared to the other two groups (Table 5). Within the cardiac myocytes, SORBS2 (a.k.a. ArgBP2) is expressed exclusively with the myofibril Z-bands, which not only link the sarcomeric contractile units together, but also regulate the signaling cascade needed for proper force production and transmission in these contractile units.^{41,42} As an adaptor protein, SORBS2 mediates interactions between structural proteins, cardiomyocyte membrane proteins, other signaling molecules, and actin filaments.⁴¹ Additionally, an important regulator of the actin cytoskeleton and apoptosis is c-Abl, whose ubiquitination and degradation is promoted by SORBS2.⁴³ Therefore, alterations in SORBS2 activity by differential phosphorylation in NIF could both disrupt the tightly regulated contractile apparatus of the heart as well as alter the apoptotic cascade, both of which can contribute to the development of heart failure. Moreover, titin, another sarcomeric protein that plays a key role in force transmission at the Z-line, was differentially phosphorylated in NIF (Table 5). The phosphorylation of these two structural proteins may contribute to the disruption of the tightly regulated contractile apparatus leading to the cardiac contractile deficiencies in NIF.

A decrease in phosphorylation of pyruvate dehydrogenase E1 component subunit α (ODPA) at serines 232, 293 and 300, indicating a significant increase in ODPA activity, was observed in IF (Table 5 and FIG. 4B).

ODPA(NCBI Reference Sequence No. NP_000275.1)

| | |
|---|---|
| 1 | MRKMLAAVSR VLSGASQKPA SRVLVASRNF ANDATFEIKK |
| | CDLHRLEEGP PVTTVLTRED |
| 61 | GLKYYRMMQT VRRMELKADQ LYKQKIIRGF CHLCDGQEAC |
| | CVGLEAGINP TDHLITAYRA |
| 121 | HGFTFTRGLS VREILAELTG RKGGCAKGKG GSMHMYAKNF |
| | YGGNGIVGAQ VPLGAGIALA |
| 181 | CKYNGKDEVC LTLYGDGAAN QGQIFEAYNM AALWKLPCIF |
| | ICENNRYGMG TSVERAAAST |
| 241 | DYYKRGDFIP GLRVDGMDIL CVREATRFAA AYCRSGKGPI |
| | LMELQTYRYH GHSMSDPGVS |
| 301 | YRTREEIQEV RSKSDPIMLL KDRMVNSNLA SVEELKEIDV |
| | EVRKEIEDAA QFATADPEPP |
| 361 | LEELGYHIYS SDPPFEVRGA NQWIKFKSVS |

This observation suggests that IF and NIF address the severe energy metabolism derangement characteristic of heart failure differently. In non-failing, well-perfused heart, fatty acids provide 60-90% of the energy for ATP production, with the remaining 10-40% derived from carbohydrate (glucose and lactate) oxidation.⁴⁴ In the failing heart, there is a switch to glucose as the preferential fuel source instead of fatty acids. The marked decrease in ODPA phosphorylation in IF suggests a mechanism by which this is occurring, namely reduction of ODPA inhibition. Interestingly, ODPA phosphorylation was not significantly changed in NIF hearts, suggesting that this shift in energy substrate is etiology specific. The trend in higher NIF pyruvate fits with the hypothesis of decreased ODPA activity in NIF compared to IF. There was lower ODPA phosphorylation in IF and lower pyruvate levels (i.e., ODPA oxidation of pyruvate was increased in IF). However, assessment of PHD activity levels did not indicate a difference between any of the sample groups. However, the insensitivity of the colorometric assay, minor extrinsic phosphorylation during tissue processing, and the complex nature of post-translation regulation of enzyme activity may explain the inability to see differences in enzyme activity levels. In all, the data suggest that ODPA activity may differentiate between NIF and IF.

### Role of Casein Kinase 2 in the Development of Heart Failure: Etiology Specific?

Of the 26 differently phosphorylated proteins, nine had sites that were likely targets of casein kinase 2 (Ck2, FIG. 7). Ck2 is one of the most ubiquitous serine/threonine protein kinases and is thought to have over 300 protein substrates that modulate a variety of cellular processes, including cell cycle control, cellular differentiation, and proliferation.⁴⁶ Classified as a “messenger-independent kinase,” Ck2 is a constitutively active kinase that is regulated through protein-protein interactions, localization, and extent of phosphorylation and oligomerization.^{46,47}

The majority of significant Ck2-specific sites in the IF hearts were increasingly phosphorylated, while all of the significant phosphoproteins with Ck2-specific sites in the NIF group were all relatively dephosphorylated. Furthermore, one of the sequences with etiology-specific differences in phosphorylation (LYRIC S298) is predicted to be a target of Ck2.¹³

A fundamental difference between the two heart failure etiologies appears to be in the activity of the pro-growth and anti-apoptotic pathways. The observation that many of these phosphorylation sites may be targets of Ck2 suggest that variation in Ck2 activity may be a crucial branching point between these two disease states. In ischemic heart failure, the initial injury and cardiomyocyte loss triggers pro-hypertrophic and anti-apoptotic pathways, of which Ck2 is a central player. This sustained cardiac hypertrophy, due in part to increased Ck2 activity, leads to maladaptive cardiac remodeling and eventually ischemic heart failure. Conversely, in non-ischemic heart disease, Ck2 activity is decreased, leading to increased apoptotic activity and suppression of pro-growth pathways. As such, augmentation of Ck2 activity could be an etiology-specific treatment.

This study is the first step in a multi-'omics-driven systems biology approach to obtain a more integrated and comprehensive molecular assessment of the different etiologies of heart failure. Using an unbiased assessment of the unenriched and phosphoenriched proteomes of two HF etiologies combined with selected metabolomics and Western blot analysis, previously undocumented molecular fingerprints of the different heart failure etiologies have been explored. These molecular differences between heart failure etiologies have enabled the addition of molecules to pathways believed to be involved in the development of HF, as well as the generation of new hypotheses to test for the mechanisms underlying disease development and progression.

### References

1. Roger et al. Heart disease and stroke statistics—2012 update: A report from the American heart association. Circulation. 2012; 125:e2-e220
2. Lloyd-Jones et al. Lifetime risk for developing congestive heart failure: The framingham heart study. Circulation. 2002; 106:3068-3072
3. Levy et al. Long-term trends in the incidence of and survival with heart failure. The New England journal of medicine. 2002; 347:1397-1402
4. Roger et al. Executive summary: Heart disease and stroke statistics—2012 update: A report from the American heart association. Circulation. 2012; 125:188-197
5. Margulies et al. Genomics, transcriptional profiling, and heart failure. Journal of the American College of Cardiology. 2009; 53:1752-1759
6. Sharma et al. Recent advances in cardiovascular proteomics. Journal of proteomics. 2012
9. Soderblom et al. Quantitative label-free phosphoproteomics strategy for multifaceted experimental designs. Analytical chemistry. 2011; 83:3758-3764
10. Weng et al. Rosetta error model for gene expression analysis. Bioinformatics. 2006; 22:1111-1121
11. Piacentino et al. X-linked inhibitor of apoptosis protein-mediated attenuation of apoptosis, using a novel cardiac-enhanced adeno-associated viral vector. Human gene therapy. 2012; 23:635-646
12. Jensen et al. Compensatory responses to pyruvate carboxylase suppression in islet beta-cells. Preservation of glucose-stimulated insulin secretion. The Journal of biological chemistry. 2006; 281:22342-22351
13. Wong et al. Kinasephos 2.0: A web server for identifying protein kinase-specific phosphorylation sites based on sequences and coupling patterns. Nucleic Acids Res. 2007; 35:W588-594
14. Jourdan-Lesaux et al. Extracellular matrix roles during cardiac repair. Life sciences. 2010; 87:391-400
15. Vilahur et al. Reperfusion-triggered stress protein response in the myocardium is blocked by post-conditioning. Systems biology pathway analysis highlights the key role of the canonical aryl-hydrocarbon receptor pathway. European heart journal. 2012
16. Bujak et al. Frangogiannis N G. Essential role of smad3 in infarct healing and in the pathogenesis of cardiac remodeling. Circulation. 2007; 116:2127-2138
17. Chaanine et al. Akt signalling in the failing heart. European journal of heart failure. 2011; 13:825-829
18. Rosello-Lleti et al. Cardiac protein changes in ischaemic and dilated cardiomyopathy: A proteomic study of human left ventricular tissue. Journal of cellular and molecular medicine. 2012; 16:2471-2486
19. Barallobre-Barreiro et al. Proteomics analysis of cardiac extracellular matrix remodeling in a porcine model of ischemia/reperfusion injury. Circulation. 2012; 125:789-802
26. Sottrup-Jensen. Alpha-macroglobulins: Structure, shape, and mechanism of proteinase complex formation. The Journal of biological chemistry. 1989; 264:11539-11542
27. French et al. Protease activation of alpha2-macroglobulin modulates a chaperone-like action with broad specificity. Biochemistry. 2008; 47:1176-1185
28. Kim et al. Dissection of tbk1 signaling via phosphoproteomics in lung cancer cells. Proceedings of the National Academy of Sciences of the United States of America. 2013; 110:12414-12419
29. Zhang et al. The oncogene metadherin modulates the apoptotic pathway based on the tumor necrosis factor superfamily member trail (tumor necrosis factor-related apoptosis-inducing ligand) in breast cancer. The Journal of biological chemistry. 2013; 288:9396-9407
39. Russ et al Inhibition of rhoa signaling with increased byes in trabecular meshwork cells. Investigative ophthalmology & visual science. 2010; 51:223-230
40. Warren et al. Myosin light chain phosphorylation is critical for adaptation to cardiac stress. Circulation. 2012; 126:2575-2588
41. Sanger et al. Arg/abl-binding protein, a z-body and z-band protein, binds sarcomeric, costameric, and signaling molecules. Cytoskeleton (Hoboken). 2010; 67:808-823
42. Frank et al. The sarcomeric z-disc: A nodal point in signalling and disease. Journal of molecular medicine. 2006; 84:446-468
43. Soubeyran et al. Cbl-argbp2 complex mediates ubiquitination and degradation of c-abl. The Biochemical journal. 2003; 370:29-34
44. Lopaschuk et al. Myocardial fatty acid metabolism in health and disease. Physiological reviews. 2010; 90:207-258
46. Bolanos-Garcia et al. Identifying interaction motifs in ck2beta—a ubiquitous kinase regulatory subunit. Trends in biochemical sciences. 2006; 31:654-661
47. Veis et al. Phosphorylation of the proteins of the extracellular matrix of mineralized tissues by casein kinase-like activity. Critical reviews in oral biology and medicine: an official publication of the American Association of Oral Biologists. 1997; 8:360-379

### Example 3

### Supplemental Materials and Methods for Example 2

### Human Cardiac Tissue Acquisition and Tissue Repository

1-2 mm thick were obtained from the anterolateral LV free wall and immediately flash frozen in liquid nitrogen and stored in a −80° C. freezer. In the ischemic hearts, the area of infarct was identified at the time of tissue procurement and only sites remote from the infarct with grossly transmural muscle and minimal scar were used in this study.

### Sample Preparation for Mass Spectrometry

Heart tissue samples were homogenized in 1 mL TRIzol (Life Technologies, Grand Island N.Y.) per 0.1 mg heart tissue. After tissue homogenization using an electric homogenizer (BioSPEC Products Inc., Model 985-370) and centrifugation, 0.8 mL of the supernatant fraction was subjected to phase separation by addition of 0.2 mL chloroform. Following centrifugation, protein was precipitated from the organic layer by addition of 1.2 mL methanol. After washing and sonication of the pellet, the protein precipitate was recovered by centrifugation and re-suspended in 0.2 mL of 0.25% w/v mass spectrometry (MS)-compatible detergent (RapiGest, Waters Corp., Milford, Mass.) in 50 mM Ammonium Bicarbonate pH 8.0. A 625 μg aliquot of protein (per sample) was subjected to reduction (10 mM dithiothreitol, 80° C. for 30 min), alkylation (20 mM iodoacetamide, RT in dark for 1 h) followed by overnight proteolysis with 1:50 w/w sequencing grade trypsin (Promega, Madison, Wis.) at 37° C. A 25 μg aliquot from each sample was used for unenriched proteomic analysis of protein expression in the heart tissue. The 25 μg unenriched proteomics profiling aliquot was acidified to 1% v/v final Trifluoroacetic acid (TFA), heated to 60° C. for 2 h, and spiked with 1.25 pmol ADH1_YEAST digest (Massprep standard, Waters Corporation) as a surrogate standard prior to analysis.

The remaining 600 μg of protein was utilized for spin-column based phosphopeptide enrichment and LC/MS/MS analysis of the phosphoproteome. Prior to phosphopeptide enrichment, each sample was spiked with trypsin digested bovine alpha-casein at 30 fmol per μg/protein lysate for use as a surrogate standard. These samples were then enriched for phosphopeptides using an in-house packed TiO₂ spin columns as previously described. (1) Briefly, samples were dried using vacuum centrifugation and re-suspended in 100 μL 80% acetonitrile, 50 mg/mL MassPrep enhancer (Waters Corp.), 1% TFA (pH 2.5). Samples were then loaded onto a TiO₂ column containing approximately 12 mg TiO₂ resin (Protea Biosciences Group, Inc., Morgantown, W. Va.) which were subsequently washed with 400 μL 80% acetonitrile, 50 mg/mL MassPrep enhancer, 1% TFA (pH 2.5) and then 400 μL 80% acetonitrile, 1% TFA (pH 2.5). Phosphopeptides were eluted using 200 μL 5% aqueous ammonia, 20% acetonitrile (pH 10.5) and were immediately acidified with neat formic acid down to pH 3.5. Samples were dried using vacuum centrifugation and then re-suspended in 2% acetonitrile, 0.1% TFA, 10 mM citric acid (pH 2.5) prior to LC/MS/MS analysis.

### LC/MS/MS Data Collection and Processing

For proteomics or unenriched samples, 1 μg of peptides were first trapped at 20 μL/min for 2 min in 99.9% water with 0.1% v/v formic acid on a 20 μm×180 mm Symmetry C18 column. Peptides were eluted from the trapping column onto a 75 μm×250 mm column with 1.7 μm C18 BEH particles (Waters, Corp.). Peptide separations were accomplished using a 90-min gradient of 5 to 40% acetonitrile (0.1% formic acid) at a flow rate of 0.3 μl/min and a 45° C. column temperature. MS and MS/MS data was collected using data-independent analysis (MS^{E}) for simultaneous peptide quantification and identification using a 0.9 s cycle time, alternating between MS (low collision energy—6 V) and MS/MS (high collision energy ramp—15 to 40 V). These qualitative/quantitative analyses were followed by an additional, supplementary qualitative LC/MS/MS experiment using data-dependent analysis (DDA) with a 0.9 s MS scan followed by MS/MS acquisition on the ‘top 3’ ions with charge greater than 1. The MS/MS acquisition for each ion used an isolation window of approximately 3 Da, a maximum of 4 s per precursor, and dynamic exclusion for 120 s (within 1.2 Da).

LTQ-Orbitrap phosphopeptide analyses were performed using the same nanoscale capillary LC column hardware and LC system that was employed for unenriched proteome analysis except that the gradient was modified by increasing the trapping time to 5 min and then a gradient hold at 5% acetonitrile (0.1% formic acid) for 5 min prior to initiating the linear gradient from 5 to 40% acetonitrile (0.1% formic acid). MS data were acquired in the Orbitrap from m/z 400-2000 with r=60,000 at m/z 400 and a target AGC setting of 1⁶ ions. The qualitative/quantitative LC/MS/MS analyses spectra utilized DDA for the ‘top 3’ precursor ions and supplementary qualitative LC/MS/MS analyses used DDA for the ‘top 10’ precursor ions. Peptide fragmentation was performed in the LTQ linear ion trap, with a CID energy setting of 35% and a dynamic exclusion of 60 s.

### LC-MS Data Processing

Unenriched and phospho-enriched proteomics datasets were independently aligned on the basis of their accurate mass and retention time. After alignment and annotation, chromatographic peak intensities belonging to the same precursor mass in the aligned chromatograms were then used to calculate the relative peptide and protein abundance on a per-sample basis. MS^{E} from the Q-ToF was used exclusively for peptide quantitation of unenriched proteomes. Protein intensities for each sample were calculated as the simple sum of the peptide intensity values. Phosphopeptide quantitation was performed on the LTQ-Orbitrap XL instrument at the peptide level from the qualitative/quantitative acquisitions.

Both MS/MS DDA and MS^{E} were used to generate peptide identifications for the unenriched analysis, and DDA exclusively for phosphopeptides. For DDA acquisition files, .mgf searchable files were produced in Rosetta Elucidator® software, and searches were then submitted to and retrieved from the Mascot v2.2 (Matrix Sciences, Boston, Mass.) search engine in an automated fashion. For MS^{E} data, ProteinLynx Global Server 2.4 (Waters Corp.) was used to generate searchable files which were then submitted to the IdentityE search engine (Waters Corp.) (2, 3) and results files were then imported back into Elucidator® software.

Both DDA and MS^{E} data were searched against the Uniprot/reviewed database with human taxonomy with full 1× reverse database appended for peptide false discovery rate determination. The final database contained 40,668 sequences including reverse entries. Q-ToF data (unenriched proteome), used a precursor ion mass tolerance of 20 ppm for both PLGS and Mascot database (DB) searches, and a product ion tolerance of 0.1 Da for Mascot and 40 ppm for PLGS. Orbitrap data (phosphoproteomics) was searched with Mascot using 10 ppm precursor and 0.8 Da product ion tolerances. Enzyme specificity was set to fully tryptic and allowed for up to 2 missed cleavages, with the exception that semi-tryptic specificity was allowed for Mascot (DDA) searches of unenriched data. Carbamidomethyl cysteine was included as a fixed modification, and variable modifications were allowed for including oxidized methionine and deamidated asparagine and glutamine. Additionally, for phosphopeptide enriched mixtures, variable phosphorylation on serine, threonine, and tyrosine was allowed.

The spectra were submitted for database searching and results were imported into Elucidator® software. To enable global spectra scoring across results from both search engines these search results were concurrently validated using the PeptideProphet and ProteinProphet algorithms in Elucidator® software using an independent reverse decoy database validation (4,5) Annotation was performed to achieve a maximum 1% FDR at the peptide level, which corresponded to a minimum PeptideProphet score of 0.6. Each peptide identified was allowed to be assigned to only a single protein entry, and these assignments were made by ProteinProphet according to the rules of parsimony. For the phosphoproteomic experiments, a mascot ion score of 26 was applied to achieve a spectral false discovery rate of 1.0%.

### Western Blot Analysis

Cryopreserved heart tissues (independent of the samples used for proteomics analysis) of each of the 12 hearts examined in the LC/MS/MS analysis were weighed and mechanically disrupted by mortar and pestle in liquid nitrogen. Pulverized heart tissues were suspended in a 5:1 volume-to-tissue weight of lysis buffer (1% IGEPAL CA-630, Sigma, 0.5% Deoxycholate, 2% SDS, 5 mM EDTA in 1×PBS) with protease and phosphatase inhibitor cocktail tablets (Roche Diagnostics, Indianapolis, Ind.). Samples were then pulse homogenized on ice with a handheld tissue tearor (BioSPEC Products Inc., Model 985-370). Homogenates were placed on ice for an additional 30 min followed by centrifugation for 30 min at 4° C. at 16,000×g (Heraeus Biofuge® Pico). Resulting supernatants were aliquoted and stored at −80° C. until analysis. Cardiac tissue homogenates were subjected to Bicinchoninic acid (BCA) assay (Pierce Biotechnology/Thermo Fisher Scientific, Rockford, Ill.) for protein quantification. Western Blot for protein immunodetection was performed using a modification of a recently published method. The primary antibodies used in this study were the following: anti-Fetuin A (#5258 Cell Signaling Technologies, Danver, Mass.), anti-fibulin 1 (ab54652 Abcam, Cambridge, Mass.), anti-ceruloplasmin (ab8813, Abcam), anti-alpha 2 macroglobulin (ab58703, Abcam), anti-carbonic anhydrase I (ab6619-1, Abcam), anti-serum amyloid A (ab687, Abcam), anti-fibulin 2 (ab66333, Abcam), anti-AKT (#9272, Cell Signaling), anti-SMAD3 (ab28379, Abcam), anti-MMP14 (ab51074, Abcam), and anti-AHR (ab28698, Abcam).

The secondary antibodies used were horseradish peroxidase conjugated anti-rabbit IgG (GE Healthcare, UK), anti-mouse IgG (Pierce), anti-sheep IgG (ab6747, Abcam), or anti-goat IgG (Sigma-Aldrich, St. Louis, Mo.). Bands were visualized using an enhanced chemiluminescence Western blotting detection system (GE Healthcare Bio-Sciences, Piscataway, N.J.). Western blots were stripped and re-probed with anti-sarcomeric actin (Sigma, A2172). The intensity of the actin band signal was used for normalization. Proteins detected were quantitated by densitometry utilizing the Image J algorithm (National Institutes of Health, Bethesda, Md.).

### References

1. Soderblom E J, Philipp M, Thompson J W, Caron M G, Moseley M A. 2011. Quantitative label-free phosphoproteomics strategy for multifaceted experimental designs. Analytical chemistry 83:3758-3764.
2. Geromanos S J, Vissers J P, Silva J C, Dorschel C A, Li G Z, Gorenstein M V, Bateman R H, Langridge J I. 2009. The detection, correlation, and comparison of peptide precursor and product ions from data independent LC-MS with data dependant LC-MS/MS. Proteomics 9:1683-1695.
3. Li G Z, Vissers J P, Silva J C, Golick D, Gorenstein M V, Geromanos S J. 2009. Database searching and accounting of multiplexed precursor and product ion spectra from the data independent analysis of simple and complex peptide mixtures. Proteomics 9:1696-1719.
4. Keller A, Nesvizhskii A I, Kolker E, Aebersold R. 2002. Empirical statistical model to estimate the accuracy of peptide identifications made by MS/MS and database search. Analytical chemistry 74:5383-5392.
5. Nesvizhskii A I, Keller A, Kolker E, Aebersold R. 2003. A statistical model for identifying proteins by tandem mass spectrometry. Analytical chemistry 75:4646-4658.

Any patents or publications mentioned in this specification are indicative of the levels of those skilled in the art to which the invention pertains. These patents and publications are herein incorporated by reference to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

TABLE 1
Demographic and clinical information.

| | | | | | % Prior | Ejection | % | |
|---|---|---|---|---|---|---|---|---|
| | Sample | Age | Sex (% | Race (% | Bypass | Fraction | Inotropic | % Intra-aortic |
| Group | Size (n) | (y ± m) | Male) | Caucasian) | Surgery | (%) | Agent | Balloon Pump |
| Non Failing (NF) | 4 | 57.0 ± 3.4 | 100 | 50 | 0 | >55% | 0 | 0 |
| Ischemic Failing (IF) | 4 | 58.0 ± 7.0 | 100 | 75 | 100 | <15% | 100 | 100 |
| Non Ischemic Failing | 4 | 51.5 ± 7.6 | 100 | 50 | 0 | <15% | 100 | 100 |
| (NIF) | | | | | | | | |

TABLE 2A
Proteins with statistical significant differential expression between IF and NF
heart tissue.
Data were filtered to show only proteins with Protein Prophet probability >0.8,
FDR-corrected p-value <0.05, and absolute fold-change >2. This is the corresponding
table for FIG. 2. Proteins in bold were validated by Western blot analysis.

| | | Peptide | Fold | p-value | |
|---|---|---|---|---|---|
| Entry Name | Protein Name | Count | Change | (ANOVA) | Protein Function |
| IGHA2_HUMAN | Ig alpha-2 chain C | 4 | 7.94 | 7.5 × 10⁻⁶ | Ig heavy chain |
| | region | | | | |
| CAH1_HUMAN | Carbonic anhydrase 1 | 2 | 7.24 | 1.6 × 10⁻³ | Acid-Base balance |
| IGHM_HUMAN | Ig mu chain C region | 2 | 4.98 | 3.1 × 10⁻³ | Ig heavy chain |
| HBA_HUMAN | Hemoglobin subunit | 12 | 3.70 | 2.6 × 10⁻² | Hb α chain |
| | alpha | | | | |
| IGHA1_HUMAN | Ig alpha-1 chain C | 17 | 3.61 | 1.3 × 10⁻³ | Ig heavy chain |
| | region | | | | |
| HBB_HUMAN | Hemoglobin subunit | 12 | 3.18 | 1.0 × 10⁻² | Hb β chain |
| | beta | | | | |
| A2MG_HUMAN | Alpha-2-macroglobulin | 33 | 2.31 | 1.0 × 10⁻⁴ | Protease Inhibitor |
| SAA_HUMAN | Serum amyloid A | 2 | −7.32 | 1.0 × 10⁻⁴ | Acute Phase Protein |
| | protein | | | | |

TABLE 2B
Proteins with statistically significant differential expression between NIF and NF heart tissue.
Data were filtered to show only proteins with Protein Prophet probability >0.8, FDR-corrected p-value <0.05
and absolute fold-change >2. Proteins in bold were validated by Western blot analysis.

| | | Peptide | Fold | p-value | |
|---|---|---|---|---|---|
| Entry Name | Protein Name | Count | Change | (ANOVA) | Protein Function |
| CAH3_HUMAN | Carbonic anhydrase 3 | 2 | 7.02 | 1.2 × 10⁻⁰² | Acid-Base balance |
| IGHA2_HUMAN | Ig alpha-2 chain C region | 4 | 4.85 | 2.6 × 10⁻⁰⁴ | Ig heavy chain |
| IGHM_HUMAN | Ig mu chain C region | 2 | 4.62 | 1.2 × 10⁻⁰³ | Ig heavy chain |
| LTBP2_HUMAN | Latent-transforming growth factor | 6 | 4.12 | 9.8 × 10⁻⁰³ | Elastic Fiber Structure |
| | β-binding protein 2 | | | | |
| CAH1_HUMAN | Carbonic anhydrase 1 | 2 | 4.06 | 3.1 × 10⁻⁰⁴ | Acid-Base balance |
| IGHA1_HUMAN | Ig alpha-1 chain C region | 17 | 3.35 | 8.0 × 10⁻⁰⁵ | Ig heavy chain |
| HBA_HUMAN | Hemoglobin subunit alpha | 12 | 3.10 | 2.6 × 10⁻⁰⁴ | Hb α chain |
| ASPN_HUMAN | Asporin | 9 | 3.03 | 1.5 × 10⁻⁰² | Cartilage Homeostasis |
| CO6A3_HUMAN | Collagen alpha-3(VI) chain | 6 | 2.93 | 5.6 × 10⁻⁰³ | ECM Fibrillar Protein |
| FBLN2_HUMAN | Fibulin-2 | 4 | 2.52 | 7.1 × 10⁻⁰³ | ECM Remodeling Protein |
| MFAP4_HUMAN | Microfibril-associated glycoprotein 4 | 5 | 2.50 | 1.4 × 10⁻⁰² | Elastic Fiber Formation |
| HBB_HUMAN | Hemoglobin subunit beta | 12 | 2.31 | 1.2 × 10⁻⁰⁴ | Hb β chain |
| FIBG_HUMAN | Fibrinogen gamma chain | 13 | 2.29 | 1.4 × 10⁰² | Primary Platelet |
| | | | | | Receptor Binding Site |
| LAC_HUMAN | Ig lambda chain C regions | 9 | 2.29 | 3.4 × 10⁻⁰⁴ | Ig light chain |
| FBLN3_HUMAN | EGF-containing fibulin-like extracellular | 2 | 2.25 | 1.7 × 10⁻⁰² | Elastic Fiber Formation |
| | matrix protein 1 | | | | |
| FBLN1_HUMAN | Fibulin-1 | 2 | 2.16 | 4.7 × 10⁻⁰² | ECM Organization |
| CERU_HUMAN | Ceruloplasmin | 15 | 2.09 | 4.0 × 10⁻⁰³ | Copper Homeostasis |
| DERM_HUMAN | Dermatopontin | 5 | 2.08 | 2.8 × 10⁻⁰² | Fibroblast Cell Adhesion |
| IGHG2_HUMAN | Ig gamma-2 chain C region | 28 | 2.01 | 1.1 × 10⁻⁰² | Ig heavy chain |
| FA9_HUMAN | Coagulation factor IX | 8 | −2.05 | 6.2 × 10⁻⁰⁴ | Coagulation Factor |
| MYH2_HUMAN | Myosin-2 | 2 | −2.08 | 4.5 × 10⁻⁰³ | Skeletal Muscle |
| | | | | | Contraction |
| ATPD_HUMAN | ATP synthase subunit delta, mitochondrial | 2 | −2.22 | 6.2 × 10⁻⁰⁴ | ATP Synthase Core |
| | | | | | Subunit |
| SAA_HUMAN | Serum amyloid A protein | 2 | −6.41 | 1.7 × 10⁻⁰⁴ | Acute Phase Protein |

TABLE 3A
Phosphopeptides with statistically significant differences in abundance in IF and NF
human heart tissue.

| | | Modified Peptide | Fold | p-value | |
|---|---|---|---|---|---|
| Entry Name | Protein Name | Sequence | Change | (ANOVA) | Protein Function |
| FETUA_HUMAN^{§} | Alpha-2-HS-glyco- | HTFMGVVSLGSPSGEVSHPR | 20.09 | 2.3 × 10⁻¹¹ | Promotes Endo- |
| | protein precursor | (SEQ ID NO: 1) | | | cytosis, |
| | | | | | Opsonization, Bone |
| FETUA_HUMAN^{\|\|} | Alpha-2-HS-glyco- | HTFMGVVSLGSPSGEVSHPR | 7.95 | 7.2 × 10⁻⁰⁴ | Mineralization |
| | protein precursor | (SEQ ID NO: 2) | | | |
| MRLC2_HUMAN | Myosin regulatory | ATSNVFAMFDQSQIQEFK | -6.30 | 3.9 × 10⁻⁰² | Muscle Contraction |
| | light chain MRLC2 | (SEQ ID NO: 3) | | | Regulation |
| LMOD1_HUMAN | Leiomodin-1 | GSPKPSPQPSPKPSPK | -6.84 | 2.0 × 10⁻⁰³ | Not well |
| | | (SEQ ID NO: 4) | | | Characterized |
| NEXN_HUMAN | Nexilin | EMLASDDEEDVSSKVEK | -16.05 | 3.1 × 10⁻⁰⁴ | Sarcomeric Z line |
| | | (SEQ ID NO: 5) | | | Protein |
| ODPA_HUMAN* | Pyruvate dehydro- | YHGHSMSDPGVSSR | -27.30 | 6.0 × 10⁻¹⁴ | Pyruvate |
| | genase E1 component | (SEQ ID NO: 6) | | | Decarboxylation |
| | subunit α, somatic | | | | |
| | form, mitochondrial | | | | |
| ODPA_HUMAN‡ | Pyruvate dehydro- | YHGHSMSDPGVSYR | -32.49 | 2.3 × 10⁻¹¹ | |
| | genase E1 component | (SEQ ID NO: 7) | | | |
| | subunit α, somatic | | | | |
| | form, mitochondrial | | | | |
| ODPA_HUMAN† | Pyruvate dehydro- | YGMGTSVER | -61.57 | 8.9 × 10⁻¹⁴ | |
| | genase E1 component | (SEQ ID NO: 8) | | | |
| | subunit α, somatic | | | | |
| | form, mitochondrial | | | | |

Data was filtered to show only proteins >6-fold and ANOVA p <0.05. Phosphorylation sites are underlined.

TABLE 3B
Phosphopeptides with statistically significant changes in abundance between
NIF and NF human heart tissues.

| | | Modified Peptide | Fold | p-value | |
|---|---|---|---|---|---|
| Entry Name | Protein Name | Sequence | Change | (ANOVA) | Protein Function |
| FETUA_HUMAN§ | Alpha-2-HS-glyco- | HTFMGVVSLGSPSGEVSHPR | 8.67 | 2.4 × 10⁻⁰⁶ | Promotes Endocytosis, |
| | protein precursor | (SEQ ID NO: 9) | | | Opsonization, Bone |
| | | | | | Mineralization |
| HS90B_HUMAN | Heat shock protein | IEDVGSDEEDDSGK | 7.48 | 6.0 × 10⁻⁰³ | Molecular Chaperone |
| | HSP 90-beta | (SEQ ID NO: 10) | | | |
| SRBS2_HUMAN** | Sorbin and SH3 | SEPAVGPPR | 7.16 | 1.8 × 10⁻⁰² | Adaptor Protein |
| | domain-containing | (SEQ ID NO: 11) | | | AKT1/PAK1 Signaling |
| | protein 2 | | | | |
| SRBS2_HUMAN^{#} | Sorbin and SH3 | DASSPVPPPHVPPPVPPLRPR | 6.98 | 6.8 × 10⁻⁰⁴ | Pathway |
| | cdomain-ontaining | (SEQ ID NO: 12) | | | |
| | protein 2 | | | | |
| POPD1_HUMAN | Blood vessel | NSIASSSDSDDGLHQFLR | -7.72 | 7.9 × 10⁻¹¹ | Cell Adhesion |
| | epicardial substance | (SEQ ID NO: 13) | | | |

Data was filtered to show only proteins differences >6-fold and which passed ANOVA p <0.05. Phosphorylation sites are underlined.

TABLE 4
Analysis of statistically significant phosphopeptides in comparison to open platform data.

| | | | Phos- | | | | Unenriched | | Unenriched | |
|---|---|---|---|---|---|---|---|---|---|---|
| | | | phorylation | | | | Fold | | P-value | |
| | | Modified | Fold Change | | Phospho P-value | | Change | | (ANOVA) | |
| Entry | Protein | Peptide | IF v | NIF v | (ANOVA) | | IF v | NIF v | IF v | NIF v |
| Name | Name | Sequence | NF | NF | IF v NF | NIF v NF | NF | NF | NF | NF |
| FETUA_HUMAN^{§} | Alpha- | HTFMGVVSLG | 20.09 | 8.67 | 2.3 × 10⁻¹¹ | 2.4 × 10⁻⁰⁶ | 1.66 | 1.71 | 1.1 × 10⁻¹¹ | 1.3 × |
| | 2-HS- | SPSGEVSHPR | | | | | | | | 10⁻⁰⁶ |
| | glyco- | (SEQ ID | | | | | | | | |
| | protein | NO: 1) | | | | | | | | |
| FETUA_HUMAN^{\|\|} | | HTFMGVVSLG | 7.95 | 4.24 | 7.2 × 10⁻⁴ | 1.1 × 10⁻⁰¹ | | | | |
| | | SPSGEVSHPR | | | | | | | | |
| | | (SEQ ID | | | | | | | | |
| | | NO: 2) | | | | | | | | |
| ODPA_HUMAN* | Pyruvate | YHGHSMS | — | -1.06 | 6.0 × 10⁻¹⁴ | 9.1 × 10⁻⁰¹ | -1.23 | -1.16 | 4.6 × 10⁻⁰¹ | 5.0 × |
| | dehydro- | DPGVSYR | 27.30 | | | | | | | 10⁻⁰¹ |
| | genase | (SEQ ID | — | | | | | | | |
| | E1 | NO: 6) | | | | | | | | |
| ODPA_HUMAN^{‡} | component | YHGHSMS | — | -1.00 | 2.3 × 10⁻¹¹ | 9.4 × 10⁻⁰¹ | | | | |
| | subunit | DPGVSYR | 32.49 | | | | | | | |
| | α, | (SEQ ID | | | | | | | | |
| | somatic | NO: 7) | | | | | | | | |
| ODPA_HUMAN^{†} | form, | YGMGTSVER | — | 2.00 | 8.9 × 10⁻¹⁴ | 7.0 × 10⁻⁰¹ | | | | |
| | mito- | (SEQ ID | 61.57 | | | | | | | |
| | chondrial | NO: 8) | | | | | | | | |
| SRBS2_HUMAN** | Sorbin | SEPAVGPPR | 2.68 | 7.16 | 4.4 × 10⁻⁰¹ | 1.8 × 10⁻⁰² | 1.01 | 1.46 | 9.4 × 10⁻⁰¹ | 2.0 × |
| | and | (SEQ ID | | | | | | | | 10⁻⁰¹ |
| | SH3 | NO: 11) | | | | | | | | |
| SRBS2_HUMAN^{#} | domain- | DASSPVPPPH | 2.64 | 6.98 | 2.4 × 10⁻⁰¹ | 6.8 × 10⁻⁰⁴ | | | | |
| | contain- | VPPPVPPLRPR | | | | | | | | |
| | ing | (SEQ ID | | | | | | | | |
| | protein | NO: 12) | | | | | | | | |
| | 2 | | | | | | | | | |
| NEXN_HUMAN | Nexilin | EMLASDDE | — | -1.19 | 3.1 × 10⁻⁰⁴ | 6.9 × 10⁻⁰¹ | -1.24 | -1.04 | 4.3 × 10⁻⁰¹ | 9.4 × |
| | | EDVSSKVEK | 16.05 | | | | | | | 10⁻⁰¹ |
| | | (SEQ ID | | | | | | | | |
| | | NO: 5) | | | | | | | | |
| MRLC2_HUMAN | Myosin | ATSNVFAMFD | -6.30 | -1.96 | 3.9 × 10⁻⁰² | 1.3 × 10⁻⁰¹ | 1.24 | 1.26 | 3.1 × 10⁻⁰¹ | 1.1 × |
| | regula- | QSQIQEFK | | | | | | | | 10⁻⁰¹ |
| | tory | (SEQ ID | | | | | | | | |
| | light | NO: 3) | | | | | | | | |
| | chain | | | | | | | | | |
| | MRLC2 | | | | | | | | | |
| HS90B_HUMAN | Heat | IEDVGSDE | 2.76 | 7.48 | 4.3 × 10⁻⁰¹ | 6.0 × 10⁻⁰³ | -1.15 | -1.08 | 6.6 × 10⁻⁰¹ | 7.7 × |
| | shock | EDDSGK | | | | | | | | 10⁻⁰¹ |
| | protein | (SEQ ID | | | | | | | | |
| | HSP | NO: 10) | | | | | | | | |
| | 90-beta | | | | | | | | | |
| POPD1_HUMAN | Blood | NSIASSSDS | 1.30 | -7.72 | 1.0 | 7.9 × 10⁻¹¹ | -1.17 | -1.40 | 5.7 × 10⁻⁰¹ | 1.5 × |
| | vessel | DDGLHQFLR | | | | | | | | 10⁻⁰¹ |
| | epicar- | (SEQ ID | | | | | | | | |
| | dial | NO: 13) | | | | | | | | |
| | substance | | | | | | | | | |

This table depicts the relationship between protein abundance and phosphorylation status of statistically significant phosphoproteins where the protein was also present in the unenriched analysis.
Values bolded in the fold change columns indicate significant fold changes defined as:
a) absolute fold change of two in the unenriched segment of the table on the right-hand side, and
b) absolute fold change >6 in the phosphorylation fold change on left-hand side.
Values bolded in the p-value column indicate statistical significance with an ANOVA generated p-value of <0.05.

TABLE 5
Phosphopeptides with statistically significant differences between
IF and NIF human heart tissue.
Phosphorylation sites are underlined.

| | | | NIF | NIF | IF | IF | NIF | NIF | |
|---|---|---|---|---|---|---|---|---|---|
| Primary | | Modified | v IF | v IF | v NF | v NF | v NF | v NF | |
| Protein | Protein | Peptide | Fold | p-value | Fold | p-value | Fold | p-value | |
| Name | Description | Sequence | Change | (ANOVA) | Change | (ANOVA) | Change | (ANOVA) | Function |
| LYRIC_ | Protein | LSSQISAGEEK | −7.29 | <0.0001 | 3.31 | 0.0002 | −2.21 | 0.0080 | Transcription |
| HUMAN | LYRIC | | | | | | | | co-activator; |
| | | | | | | | | | regulator of |
| | | | | | | | | | apoptosis |
| LMOD1_ | Leiomodin-1 | GSPKPSPQPSPKPSP | 9.66 | <0.0001 | −1.92 | 0.0001 | 5.04 | 0.0080 | Poorly |
| HUMAN | | K | | | | | | | characterized |
| | | | | | | | | | (muscle |
| | | | | | | | | | contraction?) |
| LMOD1_ | Leiomodin-1 | NSLSPATQR | 9.18 | <0.0001 | −4.93 | 0.0030 | 1.86 | 0.7820 | |
| HUMAN | | | | | | | | | |
| LMOD1_ | Leiomodin-1 | GSPKPSPQPSPKPSP | 4.40 | 0.0400 | −6.84 | 0.0020 | −1.56 | 0.7110 | |
| HUMAN | | K | | | | | | | |
| BASI_ | Basigin | KPEDVLDDDDAGSA | −7.27 | <0.0001 | 4.62 | 0.0005 | −1.57 | 0.6750 | Tissue re- |
| HUMAN | precursor | PLKSSGQHQNDK | | | | | | | modeling; cell |
| | | | | | | | | | shape & |
| | | | | | | | | | tensile |
| | | | | | | | | | properties |
| LARP7_ | La-related | KRSSSEDAESLAPR | −4.46 | 0.0100 | 3.48 | 0.0070 | −1.28 | 0.8900 | RNA |
| HUMAN | protein 7 | | | | | | | | processing; |
| | | | | | | | | | tumorgenesis |
| MLRV_ | Myosin | AGGANSNVFSMFE | −3.53 | 0.0060 | 1.70 | 0.7800 | −2.08 | 0.0880 | Cardiac muscle |
| HUMAN | regulatory | QTQIQEFK | | | | | | | contraction, |
| | light | | | | | | | | morphogenesis |
| | chain 2, | | | | | | | | |
| | ventricu- | | | | | | | | |
| | lar/cardiac | | | | | | | | |
| | muscle | | | | | | | | |
| | isoform | | | | | | | | |
| HS90A_ | Heat shock | ESEDKPEIEDVGSDE | −3.42 | 0.0002 | 3.74 | 0.0000 | 1.09 | 0.9040 | Molecular |
| HUMAN | protein | EEEKK | | | | | | | chaperone; |
| | HSP | | | | | | | | cardiac |
| | 90-alpha | | | | | | | | muscle cell |
| | | | | | | | | | apoptosis |
| ACINU_ | Apoptotic | KSSSISEEKGDSDDE | −2.15 | 0.0000 | 2.50 | 0.0000 | 1.16 | 0.8960 | Apoptosis |
| HUMAN | chromatin | KPR | | | | | | | |
| | condensation | | | | | | | | |
| | inducer in | | | | | | | | |
| | the nucleus | | | | | | | | |
| POPD1_ | Blood | NSIASSSDSDDGLH | −10.06 | <0.0001 | 1.30 | 0.9980 | −7.72 | <0.0001 | Cell |
| HUMAN | vessel | QFLR | | | | | | | adhesion & |
| | epicardial | | | | | | | | signaling |
| | substance | | | | | | | | |
| POPD1_ | Blood | GTSSMSSLHVSSPH | −8.02 | 0.0120 | 2.03 | 0.9410 | −3.96 | 0.1710 | Cell |
| HUMAN | vessel | QR | | | | | | | adhesion & |
| | epicardial | | | | | | | | signaling |
| | substance | | | | | | | | |
| FHL2_ | Four and a | YISFEER | −3.34 | 0.0130 | −1.04 | 0.9210 | −3.49 | 0.0350 | Transcription |
| HUMAN | half LIM | | | | | | | | co-activator; |
| | domains | | | | | | | | ECM assembly |
| | protein 2 | | | | | | | | |
| HSPB1_ | Heat shock | GPSWDPFRDWYPH | −3.32 | <0.0001 | −1.07 | 0.8020 | −3.56 | 0.0001 | Molecular |
| HUMAN | protein | SR | | | | | | | chaperone |
| | beta-1 | | | | | | | | |
| KAP0_ | cAMP- | TDSREDEISPPPPNP | −2.81 | 0.0040 | −1.77 | 0.3500 | −4.98 | <0.0001 | Regulation |
| HUMAN | dependent | VVK | | | | | | | of cAMP |
| | protein | | | | | | | | activity; |
| | kinase type | | | | | | | | cardiac |
| | I-alpha | | | | | | | | muscle cell |
| | regulatory | | | | | | | | proliferation |
| | subunit | | | | | | | | |
| MPRI_ | Cation- | LVSFHDDSDEDLLHI | −2.64 | 0.0120 | 1.03 | 0.9350 | −2.58 | 0.0360 | Insulin-like |
| HUMAN | independent | | | | | | | | growth factor |
| | mannose- | | | | | | | | 2 and mannose |
| | 6-phosphate | | | | | | | | 6-phosphate |
| | receptor | | | | | | | | signaling |
| SRCH_ | Sarcoplasmic | GHDGEDDEGEEEEE | −2.57 | 0.0280 | −1.36 | 0.6690 | −3.50 | 0.0040 | Calcium |
| HUMAN | reticulum | EEEEEEEASTEYGHQ | | | | | | | homeostasis; |
| | histidine- | AHR | | | | | | | regulation |
| | rich | | | | | | | | of heart |
| | calcium- | | | | | | | | contraction |
| | binding | | | | | | | | |
| | protein | | | | | | | | |
| KCRM_ | Creatine | GTGGVDTAAVGSVF | −2.11 | 0.0370 | −1.79 | 0.5830 | −3.77 | 0.0001 | Energy homeo- |
| HUMAN | kinase | DVSNADR | | | | | | | stasis; |
| | M-type | | | | | | | | biomarker |
| | | | | | | | | | for myocardial |
| | | | | | | | | | infarction |
| SRBS2_ | Sorbin and | SFTSSSPSSPSR | 5.55 | <0.0001 | −1.69 | 0.2940 | 3.28 | 0.0350 | Z-band |
| HUMAN | SH3 domain- | | | | | | | | signaling; |
| | containing | | | | | | | | cytoskeleton |
| | protein 2 | | | | | | | | regulation |
| AKAl2_ | A-kinase | EGVTPWASFKK | 4.43 | 0.0030 | −1.02 | 0.9800 | 4.33 | 0.0080 | Cell growth; |
| HUMAN | anchor | | | | | | | | signal |
| | protein 12 | | | | | | | | transduction |
| SRBS2_ | Sorbin and | TSPGRVDLPGSSTTL | 3.70 | <0.0001 | 1.04 | 0.9970 | 3.85 | 0.0004 | Z-band |
| HUMAN | SH3 domain- | TK | | | | | | | signaling; |
| | containing | | | | | | | | cytoskeleton |
| | protein 2 | | | | | | | | regulation |
| MAP4_ | Microtubule- | VGSLDNVGHLPAGG | 3.32 | 0.0190 | 1.37 | 0.5590 | 4.55 | <0.0001 | Cell cycle |
| HUMAN | associated | AVK | | | | | | | progression |
| | protein 4 | | | | | | | | |
| SRBS2_ | Sorbin and | TSPGRVDLPGSSTTL | 3.15 | 0.0010 | −1.07 | 0.8410 | 2.95 | 0.0060 | Z-band |
| HUMAN | SH3 domain- | TK | | | | | | | signaling; |
| | containing | | | | | | | | cytoskeleton |
| | protein 2 | | | | | | | | regulation |
| TITIN_ | Titin | SRSTPPSIAAK | 3.10 | 0.0300 | 1.73 | 0.5250 | 5.36 | 0.0001 | Cardiac muscle |
| HUMAN | | | | | | | | | development & |
| | | | | | | | | | contraction; |
| | | | | | | | | | tissue |
| | | | | | | | | | elasticity |
| LMO7_ | LIM domain | RGESLDNLDSPR | 2.79 | 0.0340 | 1.32 | 0.7820 | 3.67 | 0.0040 | Cell adhesion |
| HUMAN | only protein | | | | | | | | |
| | 7 | | | | | | | | |
| SRBS2_ | Sorbin and | DASSPVPPPHVPPP | 2.64 | 0.0140 | 2.64 | 0.2380 | 6.98 | 0.0007 | Z-band |
| HUMAN | SH3 domain- | VPPLRPR | | | | | | | signaling; |
| | containing | | | | | | | | cytoskeleton |
| | protein 2 | | | | | | | | regulation |
| MATR3_ | Matrin-3 | SYSPDGKESPSDKK | 2.61 | 0.0060 | −1.11 | 0.0003 | 2.36 | 0.0750 | Cell growth & |
| HUMAN | | | | | | | | | proliferation; |
| | | | | | | | | | DNA damage |
| | | | | | | | | | response |

TABLE 6
Significant canonical pathways for ischemic and non-ischemic heart failure compared to normal heart tissue.

| | | IF v NF | IF v NF p- | NIF v NF | NIF v NF p- |
|---|---|---|---|---|---|
| | | Fold | value | Fold | value |
| Symbol | Entrez Gene Name | Change | (ANOVA) | Change | (ANOVA) |
| Mitochondrial Dysfunction | | | 2.97E−13* | | 4.54E−07* |
| ATP5C1 | ATP synthase, H+ transporting, mitochondrial F1 complex, gamma polypeptide | −1.471 | 2.80E−03 | −1.295 | 1.40E−02 |
| | 1 | | | | |
| ATP5D | ATP synthase, H+ transporting, mitochondrial F1 complex, delta subunit | NS | | −2.216 | 6.16E−04 |
| COX4I1 | cytochrome c oxidase subunit IV isoform 1 | −1.42 | 3.80E−03 | NS | |
| COX5A | cytochrome c oxidase subunit Va | −1.532 | 1.80E−03 | NS | |
| COX5B | cytochrome c oxidase subunit Vb | −1.406 | 1.24E−02 | NS | |
| COX6B1 | cytochrome c oxidase subunit VIb polypeptide 1 (ubiquitous) | −1.373 | 2.11E−02 | NS | |
| NDUFA4 | NADH dehydrogenase (ubiquinone) 1 alpha subcomplex, 4, 9 kDa | NS | | −1.595 | 4.93E−02 |
| NDUFA8 | NADH dehydrogenase (ubiquinone) 1 alpha subcomplex, 8, 19 kDa | −1.38 | 4.69E−02 | NS | |
| NDUFA12 | NADH dehydrogenase (ubiquinone) 1 alpha subcomplex, 12 | −1.526 | 8.40E−03 | -1.613 | 2.17E−02 |
| NDUFB10 | NADH dehydrogenase (ubiquinone) 1 beta subcomplex, 10, 22 kDa | −1.366 | 3.50E−03 | NS | |
| NDUFS4 | NADH dehydrogenase (ubiquinone) Fe—S protein 4, 18 kDa (NADH-coenzyme Q | NS | | −1.477 | 2.29E−02 |
| | reductase) | | | | |
| NDUFS6 | NADH dehydrogenase (ubiquinone) Fe—S protein 6, 13 kDa (NADH-coenzyme Q | NS | | −1.581 | 1.18E−02 |
| | reductase) | | | | |
| PARK7 | parkinson protein 7 | 1.366 | 1.66E−02 | 1.579 | 2.75E−07 |
| SOD2 | superoxide dismutase 2, mitochondrial | −1.332 | 1.00E−02 | NS | |
| UQCRFS1 | ubiquinol-cytochrome c reductase, Rieske iron-sulfur polypeptide 1 | −1.333 | 2.03E−02 | −1.397 | 2.40E−03 |
| UQCRH | ubiquinol-cytochrome c reductase hinge protein | −1.356 | 2.80E−03 | −1.588 | 4.09E−02 |
| Calcium Signaling | | | 2.05E−07* | | 1.08E−07* |
| CALR | calreticulin | NS | | −1.198 | 6.91E−04 |
| CASQ2 | calsequestrin 2 (cardiac muscle) | NS | | −1.273 | 2.89E−02 |
| MYH2 | myosin, heavy chain 2, skeletal muscle, adult | NS | | −2.077 | 4.50E−03 |
| MYH7 | myosin, heavy chain 7, cardiac muscle, beta | −1.352 | 2.46E−02 | NS | |
| MYL1 | myosin, light chain 1, alkali; skeletal, fast | NS | | −1.197 | 4.22E−02 |
| MYL2 | myosin, light chain 2, regulatory, cardiac, slow | −1.285 | 2.80E−03 | NS | |
| MYL3 | myosin, light chain 3, alkali; ventricular, skeletal, slow | −1.251 | 1.90E−03 | NS | |
| MYL4 | myosin, light chain 4, alkali; atrial, embryonic | 1.593 | 2.50E−03 | 1.68 | 5.10E−03 |
| TNNC1 | troponin C type 1 (slow) | −1.221 | 2.30E−03 | −1.364 | 2.40E−03 |
| TNNT2 | troponin T type 2 (cardiac) | −1.225 | 2.23E−02 | −1.418 | 1.20E−05 |
| TPM1 | tropomyosin 1 (alpha) | −1.162 | 1.63E−02 | −1.266 | 2.48E−04 |
| TPM2 | tropomyosin 2 (beta) | −1.229 | 2.70E−03 | −1.348 | 7.73E−06 |
| TPM3 | tropomyosin 3 | NS | | −1.325 | 8.90E−03 |
| Acute Phase Response Signaling | | | 1.62E−06* | | 5.27E−07* |
| A2M | alpha-2-macroglobulin | 2.31 | 1.01E−04 | 1.843 | 2.05E−04 |
| AHSG | alpha-2-HS-glycoprotein | 1.665 | 1.07E−11 | 1.708 | 1.34E−06 |
| ALB | albumin | 1.364 | 5.02E−07 | NS | |
| CP | ceruloplasmin (ferroxidase) | 1.829 | 6.30E−03 | 2.092 | 4.00E−03 |
| FGB | fibrinogen beta chain | NS | | 1.964 | 2.17E−02 |
| FGG | fibrinogen gamma chain | NS | | 2.293 | 1.42E−02 |
| HRG | histidine-rich glycoprotein | 1.652 | 1.32E−02 | 1.558 | 2.17E−02 |
| SOD2 | superoxide dismutase 2, mitochondrial | −1.332 | 1.00E−02 | NS | |
| TF | transferrin | 1.979 | 2.78E−05 | 1.81 | 4.54E−04 |
| TTR | transthyretin | NS | | 1.528 | 1.19E−02 |

*p-value listed is the association between the datasets for each HF etiology and the pathway listed (calculated via Ingenuity Pathway Analysis)

TABLE 7
UNENRICHED PROTEIN EXPRESSION PROFILES

| | | | | NIF v IF |
|---|---|---|---|---|
| Primary Protein | | ProteinTeller | Peptide | Fold |
| Name | Protein Description | Probability | Count | Change |
| 1433B_HUMAN | 14-3-3 protein beta/alpha | 1 | 11 | 1.02 |
| | OS = Homo sapiens GN = YWHAB | | | |
| | PE = 1 SV = 3 | | | |
| 1433E_HUMAN | 14-3-3 protein epsilon OS = | 1 | 7 | 1.02 |
| | Homo sapiens GN = YWHAE PE = 1 | | | |
| | SV = 1 | | | |
| 1433F_HUMAN | 14-3-3 protein eta OS = | 0.87 | 2 | −1.24 |
| | Homo sapiens GN = YWHAH PE = 1 | | | |
| | SV = 4 | | | |
| 1433G_HUMAN | 14-3-3 protein gamma OS = | 1 | 10 | 1.14 |
| | Homo sapiens GN = YWHAG PE = 1 | | | |
| | SV = 2 | | | |
| 1433T_HUMAN | 14-3-3 protein theta OS = | 1 | 5 | −1.11 |
| | Homo sapiens GN = YWHAQ PE = 1 | | | |
| | SV = 1 | | | |
| 1433Z_HUMAN | 14-3-3 protein zeta/delta | 1 | 7 | 1.08 |
| | OS = Homo sapiens GN = YWHAZ | | | |
| | PE = 1 SV = 1 | | | |
| A1AG1_HUMAN | Alpha-1-acid glycoprotein 1 | 1 | 13 | −1.14 |
| | OS = Homo sapiens GN = ORM1 | | | |
| | PE = 1 SV = 1 | | | |
| A1AG2_HUMAN | Alpha-1-acid glycoprotein 2 | 1 | 21 | −1.16 |
| | OS = Homo sapiens GN = ORM2 | | | |
| | PE = 1 SV = 2 | | | |
| A1AT_HUMAN | Alpha-1-antitrypsin OS = | 1 | 57 | −1.03 |
| | Homo sapiens GN = SERPINA1 PE = 1 | | | |
| | SV = 3 | | | |
| A1BG_HUMAN | Alpha-1B-glycoprotein OS = | 1 | 19 | −1.20 |
| | Homo sapiens GN = A1BG PE = 1 | | | |
| | SV = 3 | | | |
| A26CA_HUMAN | ANKRD26-like family C member | 1 | 32 | −1.01 |
| | 1A - Homo sapiens | | | |
| A26CB_HUMAN | ANKRD26-like family C member | 1 | 8 | 1.09 |
| | 1B - Homo sapiens | | | |
| A2GL_HUMAN | Leucine-rich alpha-2-glycoprotein | 1 | 12 | −1.46 |
| | OS = Homo sapiens GN = LRG1 PE = 1 | | | |
| | SV = 2 | | | |
| A2MG_HUMAN | Alpha-2-macroglobulin OS = | 1 | 33 | −1.25 |
| | Homo sapiens GN = A2M PE = 1 | | | |
| | SV = 1 | | | |
| AACT_HUMAN | Alpha-1-antichymotrypsin | 1 | 26 | −1.11 |
| | OS = Homo sapiens GN = SERPINA3 | | | |
| | PE = 1 SV = 2 | | | |
| AATC_HUMAN | Aspartate aminotransferase, | 0.84 | 2 | −1.15 |
| | cytoplasmic OS = Homo sapiens | | | |
| | GN = GOT1 PE = 1 SV = 3 | | | |
| ABCAD_HUMAN | ATP-binding cassette sub-family A | 0 | 1 | −1.02 |
| | member 13 OS = Homo sapiens | | | |
| | GN = ABCA13 PE = 2 SV = 2 | | | |
| ACADM_HUMAN | Medium-chain specific acyl-CoA | 1 | 5 | 1.36 |
| | dehydrogenase, mitochondrial | | | |
| | OS = Homo sapiens GN = ACADM | | | |
| | PE = 1 SV = 1 | | | |
| ACPM_HUMAN | Acyl carrier protein, | 1 | 6 | −1.04 |
| | mitochondrial OS = Homo sapiens | | | |
| | GN = NDUFAB1 PE = 1 SV = 3 | | | |
| ACTA_HUMAN | Actin, aortic smooth muscle | 1 | 10 | −1.00 |
| | OS = Homo sapiens GN = ACTA2 | | | |
| | PE = 1 SV = 1 | | | |
| ACTB_HUMAN | Actin, cytoplasmic 1 OS = | 1 | 10 | 1.27 |
| | Homo sapiens GN = ACTB PE = 1 | | | |
| | SV = 1 | | | |
| ACTBL_HUMAN | Beta-actin-like protein 2 | 1 | 12 | 1.04 |
| | OS = Homo sapiens GN = ACTBL2 | | | |
| | PE = 1 SV = 2 | | | |
| ACTK_HUMAN | Kappa-actin - Homo sapiens | 1 | 8 | 1.04 |
| ACTS_HUMAN | Actin, alpha skeletal muscle | 1 | 49 | 1.04 |
| | OS = Homo sapiens GN = ACTA1 | | | |
| | PE = 1 SV = 1 | | | |
| ADH1_YEAST | Alcohol dehydrogenase 1 | 1 | 21 | 1.04 |
| | OS = Saccharomyces cerevisiae | | | |
| | GN = ADH1 PE = 1 SV = 4 | | | |
| ADH1G_HUMAN | Alcohol dehydrogenase 1C | 0 | 1 | −1.04 |
| | OS = Homo sapiens GN = ADH1C | | | |
| | PE = 1 SV = 2 | | | |
| ADIP_HUMAN | Afadin- and alpha-actinin-binding | 0 | 1 | −1.35 |
| | protein OS = Homo sapiens | | | |
| | GN = SSX2IP PE = 1 SV = 3 | | | |
| ADIPO_HUMAN | Adiponectin OS = Homo sapiens | 0 | 1 | −1.01 |
| | GN = ADIPOQ PE = 1 SV = 1 | | | |
| AHNK2_HUMAN | Protein AHNAK2 OS = | 0.55 | 1 | −1.17 |
| | Homo sapiens GN = AHNAK2 PE = 1 | | | |
| | SV = 2 | | | |
| AIFM1_HUMAN | Apoptosis-inducing factor 1, | 0.91 | 1 | 1.08 |
| | mitochondrial OS = Homo sapiens | | | |
| | GN = AIFM1 PE = 1 SV = 1 | | | |
| AIM1_HUMAN | Absent in melanoma 1 protein | 0.34 | 1 | −1.36 |
| | OS = Homo sapiens GN = AIM1 | | | |
| | PE = 1 SV = 3 | | | |
| ALBU_HUMAN | Serum albumin OS = Homo sapiens | 1 | 242 | 1.07 |
| | GN = ALB PE = 1 SV = 2 | | | |
| ALDOA_HUMAN | Fructose-bisphosphate aldolase A | 1 | 24 | 1.10 |
| | OS = Homo sapiens GN = ALDOA | | | |
| | PE = 1 SV = 2 | | | |
| ALDOC_HUMAN | Fructose-bisphosphate aldolase C | 1 | 12 | −1.01 |
| | OS = Homo sapiens GN = ALDOC | | | |
| | PE = 1 SV = 2 | | | |
| ALPK2_HUMAN | Alpha-protein kinase 2 OS = | 0 | 1 | 1.27 |
| | Homo sapiens GN = ALPK2 PE = 1 | | | |
| | SV = 3 | | | |
| AMBP_HUMAN | Protein AMBP OS = Homo sapiens | 1 | 9 | −1.42 |
| | GN = AMBP PE = 1 SV = 1 | | | |
| ANGT_HUMAN | Angiotensinogen OS = | 0.83 | 1 | 1.21 |
| | Homo sapiens GN = AGT PE = 1 | | | |
| | SV = 1 | | | |
| ANT3_HUMAN | Antithrombin-III OS = | 1 | 16 | −1.32 |
| | Homo sapiens GN = SERPINC1 PE = 1 | | | |
| | SV = 1 | | | |
| ANXA2_HUMAN | Annexin A2 OS = Homo sapiens | 1 | 5 | −1.00 |
| | GN = ANXA2 PE = 1 SV = 2 | | | |
| ANXA5_HUMAN | Annexin A5 OS = Homo sapiens | 1 | 6 | 1.13 |
| | GN = ANXA5 PE = 1 SV = 2 | | | |
| AOC3_HUMAN | Membrane primary amine | 1 | 9 | −1.02 |
| | oxidase OS = Homo sapiens | | | |
| | GN = AOC3 PE = 1 SV = 3 | | | |
| APOA4_HUMAN | Apolipoprotein A-IV OS = | 0.31 | 1 | −1.07 |
| | Homo sapiens GN = APOA4 PE = 1 | | | |
| | SV = 3 | | | |
| APOH_HUMAN | Beta-2-glycoprotein 1 OS = | 1 | 17 | −1.21 |
| | Homo sapiens GN = APOH PE = 1 | | | |
| | SV = 3 | | | |
| APOOL_HUMAN | Apolipoprotein O-like OS = | 0.83 | 1 | 1.04 |
| | Homo sapiens GN = APOOL PE = 1 | | | |
| | SV = 1 | | | |
| ARGFX_HUMAN | Arginine-fifty homeobox | 0 | 1 | −1.39 |
| | OS = Homo sapiens GN = ARGFX | | | |
| | PE = 2 SV = 1 | | | |
| ARHGH_HUMAN | Rho guanine nucleotide exchange | 0.68 | 1 | −1.17 |
| | factor 17 OS = Homo sapiens | | | |
| | GN = ARHGEF17 PE = 1 SV = 1 | | | |
| ARHGJ_HUMAN | Rho guanine nucleotide exchange | 0 | 1 | 1.13 |
| | factor 19 OS = Homo sapiens | | | |
| | GN = ARHGEF19 PE = 2 SV = 1 | | | |
| ASAH1_HUMAN | Acid ceramidase OS = | 1 | 22 | −1.29 |
| | Homo sapiens GN = ASAH1 PE = 1 | | | |
| | SV = 5 | | | |
| ASPN_HUMAN | Asporin OS = Homo sapiens | 1 | 9 | 1.64 |
| | GN = ASPN PE = 1 SV = 2 | | | |
| AT1B1_HUMAN | Sodium/potassium-transporting | 1 | 14 | −1.17 |
| | ATPase subunit beta-1 OS = | | | |
| | Homo sapiens GN = ATP1B1 PE = 1 | | | |
| | SV = 1 | | | |
| ATPB_HUMAN | ATP synthase subunit beta, | 1 | 13 | 1.02 |
| | mitochondrial OS = Homo sapiens | | | |
| | GN = ATP5B PE = 1 SV = 3 | | | |
| ATPD_HUMAN | ATP synthase subunit delta, | 0.97 | 2 | −1.34 |
| | mitochondrial OS = Homo sapiens | | | |
| | GN = ATP5D PE = 1 SV = 2 | | | |
| ATPG_HUMAN | ATP synthase subunit gamma, | 1 | 6 | 1.14 |
| | mitochondrial OS = Homo sapiens | | | |
| | GN = ATP5C1 PE = 1 SV = 1 | | | |
| ATPK_HUMAN | ATP synthase subunit f, | 0.83 | 1 | −1.11 |
| | mitochondrial OS = Homo sapiens | | | |
| | GN = ATP5J2 PE = 1 SV = 3 | | | |
| ATS20_HUMAN | A disintegrin and | 0 | 1 | 1.05 |
| | metalloproteinase with | | | |
| | thrombospondin motifs 20 | | | |
| | OS = Homo sapiens | | | |
| | GN = ADAMTS20 PE = 2 SV = 2 | | | |
| AXA2L_HUMAN | Putative annexin A2-like protein | 1 | 18 | 1.05 |
| | OS = Homo sapiens GN = ANXA2P2 | | | |
| | PE = 5 SV = 2 | | | |
| BASI_HUMAN | Basigin OS = Homo sapiens | 1 | 14 | −1.13 |
| | GN = BSG PE = 1 SV = 2 | | | |
| BIEA_HUMAN | Biliverdin reductase A OS = | 0.8 | 2 | −1.04 |
| | Homo sapiens GN = BLVRA PE = 1 | | | |
| | SV = 2 | | | |
| BST2_HUMAN | Bone marrow stromal antigen 2 | 0.83 | 1 | −1.18 |
| | OS = Homo sapiens GN = BST2 PE = 1 | | | |
| | SV = 1 | | | |
| BUD13_HUMAN | BUD13 homolog OS = | 0.31 | 1 | 1.03 |
| | Homo sapiens GN = BUD13 PE = 1 | | | |
| | SV = 1 | | | |
| C1QBP_HUMAN | Complement component 1 Q | 1 | 10 | −1.14 |
| | subcomponent-binding protein, | | | |
| | mitochondrial OS = Homo sapiens | | | |
| | GN = C1QBP PE = 1 SV = 1 | | | |
| CAD13_HUMAN | Cadherin-13 OS = Homo sapiens | 1 | 22 | −1.11 |
| | GN = CDH13 PE = 1 SV = 1 | | | |
| CADH2_HUMAN | Cadherin-2 OS = Homo sapiens | 1 | 24 | −1.15 |
| | GN = CDH2 PE = 1 SV = 4 | | | |
| CAH1_HUMAN | Carbonic anhydrase 1 OS = | 0.98 | 2 | −1.78 |
| | Homo sapiens GN = CA1 PE = 1 | | | |
| | SV = 2 | | | |
| CAH3_HUMAN | Carbonic anhydrase 3 OS = | 0.97 | 2 | 2.83 |
| | Homo sapiens GN = CA3 PE = 1 | | | |
| | SV = 3 | | | |
| CALD1_HUMAN | Caldesmon OS = Homo sapiens | 1 | 6 | 1.17 |
| | GN = CALD1 PE = 1 SV = 2 | | | |
| CALM_HUMAN | Calmodulin OS = Homo sapiens | 1 | 16 | 1.02 |
| | GN = CALM1 PE = 1 SV = 2 | | | |
| CALR_HUMAN | Calreticulin OS = Homo sapiens | 1 | 10 | −1.08 |
| | GN = CALR PE = 1 SV = 1 | | | |
| CALU_HUMAN | Calumenin OS = Homo sapiens | 0.98 | 2 | −1.18 |
| | GN = CALU PE = 1 SV = 2 | | | |
| CAPZB_HUMAN | F-actin-capping protein subunit | 1 | 2 | 1.02 |
| | beta OS = Homo sapiens | | | |
| | GN = CAPZB PE = 1 SV = 4 | | | |
| CASQ2_HUMAN | Calsequestrin-2 OS = | 1 | 33 | −1.23 |
| | Homo sapiens GN = CASQ2 PE = 1 | | | |
| | SV = 2 | | | |
| CATB_HUMAN | Cathepsin B OS = Homo sapiens | 1 | 6 | 1.06 |
| | GN = CTSB PE = 1 SV = 3 | | | |
| CATD_HUMAN | Cathepsin D OS = Homo sapiens | 1 | 29 | −1.03 |
| | GN = CTSD PE = 1 SV = 1 | | | |
| CAZA2_HUMAN | F-actin-capping protein subunit | 0.61 | 1 | 1.11 |
| | alpha-2 OS = Homo sapiens | | | |
| | GN = CAPZA2 PE = 1 SV = 3 | | | |
| CCD57_HUMAN | Coiled-coil domain-containing | 0.32 | 1 | −1.24 |
| | protein 57 OS = Homo sapiens | | | |
| | GN = CCDC57 PE = 2 SV = 1 | | | |
| CD36_HUMAN | Platelet glycoprotein 4 OS = | 1 | 19 | −1.44 |
| | Homo sapiens GN = CD36 PE = 1 | | | |
| | SV = 2 | | | |
| CD59_HUMAN | CD59 glycoprotein OS = | 1 | 8 | −1.09 |
| | Homo sapiens GN = CD59 PE = 1 | | | |
| | SV = 1 | | | |
| CD99_HUMAN | CD99 antigen OS = Homo sapiens | 0.83 | 1 | −1.24 |
| | GN = CD99 PE = 1 SV = 1 | | | |
| CERU_HUMAN | Ceruloplasmin OS = Homo sapiens | 1 | 15 | 1.14 |
| | GN = CP PE = 1 SV = 1 | | | |
| CFAB_HUMAN | Complement factor B OS = | 1 | 24 | −1.05 |
| | Homo sapiens GN = CFB PE = 1 | | | |
| | SV = 2 | | | |
| CFAH_HUMAN | Complement factor H OS = | 1 | 18 | −1.10 |
| | Homo sapiens GN = CFH PE = 1 | | | |
| | SV = 4 | | | |
| CH10_HUMAN | 10 kDa heat shock protein, | 1 | 9 | 1.07 |
| | mitochondrial OS = Homo sapiens | | | |
| | GN = HSPE1 PE = 1 SV = 2 | | | |
| CHCH3_HUMAN | Coiled-coil-helix-coiled-coil-helix | 1 | 14 | −1.10 |
| | domain-containing protein 3, | | | |
| | mitochondrial OS = Homo sapiens | | | |
| | GN = CHCHD3 PE = 1 SV = 1 | | | |
| CHDH_HUMAN | Choline dehydrogenase, | 0 | 1 | −1.21 |
| | mitochondrial OS = Homo sapiens | | | |
| | GN = CHDH PE = 2 SV = 1 | | | |
| CHST7_HUMAN | Carbohydrate sulfotransferase 7 | 0 | 1 | −1.56 |
| | OS = Homo sapiens GN = CHST7 | | | |
| | PE = 1 SV = 2 | | | |
| CISD1_HUMAN | CDGSH iron sulfur domain- | 1 | 4 | −1.16 |
| | containing protein 1 OS = | | | |
| | Homo sapiens GN = CISD1 PE = 1 | | | |
| | SV = 1 | | | |
| CK067_HUMAN | UPF0366 protein C11orf67 | 0.98 | 2 | −1.33 |
| | OS = Homo sapiens GN = C11orf67 | | | |
| | PE = 1 SV = 1 | | | |
| CLCB_HUMAN | Clathrin light chain B OS = | 0.83 | 1 | −1.13 |
| | Homo sapiens GN = CLTB PE = 1 | | | |
| | SV = 1 | | | |
| CLUS_HUMAN | Clusterin OS = Homo sapiens | 1 | 7 | −1.23 |
| | GN = CLU PE = 1 SV = 1 | | | |
| CMA1_HUMAN | Chymase OS = Homo sapiens | 1 | 3 | 1.37 |
| | GN = CMA1 PE = 1 SV = 1 | | | |
| CN045_HUMAN | Uncharacterized protein | 0 | 1 | 1.01 |
| | C14orf45 OS = Homo sapiens | | | |
| | GN = C14orf45 PE = 1 SV = 3 | | | |
| CNBP_HUMAN | Cellular nucleic acid-binding | 0.83 | 1 | −1.18 |
| | protein OS = Homo sapiens | | | |
| | GN = CNBP PE = 1 SV = 1 | | | |
| CO4A1_HUMAN | Collagen alpha-1(IV) chain | 0.83 | 1 | −1.01 |
| | OS = Homo sapiens GN = COL4A1 | | | |
| | PE = 1 SV = 3 | | | |
| CO4A2_HUMAN | Collagen alpha-2(IV) chain | 0.91 | 2 | 1.24 |
| | OS = Homo sapiens GN = COL4A2 | | | |
| | PE = 1 SV = 4 | | | |
| CO6A1_HUMAN | Collagen alpha-1(VI) chain | 1 | 9 | 1.13 |
| | OS = Homo sapiens GN = COL6A1 | | | |
| | PE = 1 SV = 3 | | | |
| CO6A2_HUMAN | Collagen alpha-2(VI) chain | 0.61 | 1 | 1.34 |
| | OS = Homo sapiens GN = COL6A2 | | | |
| | PE = 1 SV = 4 | | | |
| CO6A3_HUMAN | Collagen alpha-3(VI) chain | 1 | 6 | 1.67 |
| | OS = Homo sapiens GN = COL6A3 | | | |
| | PE = 1 SV = 4 | | | |
| CO9_HUMAN | Complement component C9 | 1 | 6 | −1.15 |
| | OS = Homo sapiens GN = C9 PE = 1 | | | |
| | SV = 2 | | | |
| COF2_HUMAN | Cofilin-2 OS = Homo sapiens | 0.83 | 1 | 1.58 |
| | GN = CFL2 PE = 1 SV = 1 | | | |
| COFA1_HUMAN | Collagen alpha-1(XV) chain | 1 | 9 | −1.23 |
| | OS = Homo sapiens GN = COL15A1 | | | |
| | PE = 1 SV = 2 | | | |
| COG1_HUMAN | Conserved oligomeric Golgi | 0.81 | 2 | 1.26 |
| | complex subunit 1 OS = | | | |
| | Homo sapiens GN = COG1 PE = 1 | | | |
| | SV = 1 | | | |
| COIA1_HUMAN | Collagen alpha-1(XVIII) chain | 1 | 3 | −1.00 |
| | OS = Homo sapiens GN = COL18A1 | | | |
| | PE = 1 SV = 5 | | | |
| CORL2_HUMAN | Ladybird homeobox corepressor | 0 | 1 | 1.21 |
| | 1-like protein OS = Homo sapiens | | | |
| | GN = CORL2 PE = 1 SV = 2 | | | |
| COX2_HUMAN | Cytochrome c oxidase subunit 2 | 0.98 | 3 | 1.19 |
| | OS = Homo sapiens GN = MT-CO2 | | | |
| | PE = 1 SV = 1 | | | |
| COX41_HUMAN | Cytochrome c oxidase subunit 4 | 1 | 15 | 1.06 |
| | isoform 1, mitochondrial | | | |
| | OS = Homo sapiens GN = COX4I1 | | | |
| | PE = 1 SV = 1 | | | |
| COX5A_HUMAN | Cytochrome c oxidase subunit 5A, | 1 | 14 | 1.09 |
| | mitochondrial OS = Homo sapiens | | | |
| | GN = COX5A PE = 1 SV = 2 | | | |
| COX5B_HUMAN | Cytochrome c oxidase subunit 5B, | 1 | 4 | 1.07 |
| | mitochondrial OS = Homo sapiens | | | |
| | GN = COX5B PE = 1 SV = 2 | | | |
| CRIP2_HUMAN | Cysteine-rich protein 2 OS = | 1 | 11 | −1.00 |
| | Homo sapiens GN = CRIP2 PE = 1 | | | |
| | SV = 1 | | | |
| CRYAB_HUMAN | Alpha-crystallin B chain OS = | 1 | 12 | −1.10 |
| | Homo sapiens GN = CRYAB PE = 1 | | | |
| | SV = 2 | | | |
| CSPG2_HUMAN | Versican core protein OS = | 1 | 9 | 1.50 |
| | Homo sapiens GN = VCAN PE = 1 | | | |
| | SV = 3 | | | |
| CSRP1_HUMAN | Cysteine and glycine-rich protein | 0.83 | 1 | 1.29 |
| | 1 OS = Homo sapiens GN = CSRP1 | | | |
| | PE = 1 SV = 3 | | | |
| CSRP3_HUMAN | Cysteine and glycine-rich protein | 1 | 39 | −1.11 |
| | 3 OS = Homo sapiens GN = CSRP3 | | | |
| | PE = 1 SV = 1 | | | |
| CX6B1_HUMAN | Cytochrome c oxidase subunit | 1 | 4 | 1.09 |
| | 6B1 OS = Homo sapiens | | | |
| | GN = COX6B1 PE = 1 SV = 2 | | | |
| CX7A1_HUMAN | Cytochrome c oxidase | 0.83 | 1 | −1.01 |
| | polypeptide 7A1, mitochondrial | | | |
| | OS = Homo sapiens GN = COX7A1 | | | |
| | PE = 1 SV = 2 | | | |
| CY1_HUMAN | Cytochrome c1, heme protein, | 1 | 5 | −1.09 |
| | mitochondrial OS = Homo sapiens | | | |
| | GN = CYC1 PE = 1 SV = 2 | | | |
| CYC_HUMAN | Cytochrome c OS = Homo sapiens | 1 | 36 | −1.03 |
| | GN = CYCS PE = 1 SV = 2 | | | |
| DAG1_HUMAN | Dystroglycan OS = Homo sapiens | 1 | 7 | −1.19 |
| | GN = DAG1 PE = 1 SV = 2 | | | |
| DECR_HUMAN | 2,4-dienoyl-CoA reductase, | 0.96 | 1 | 1.16 |
| | mitochondrial OS = Homo sapiens | | | |
| | GN = DECR1 PE = 1 SV = 1 | | | |
| DERM_HUMAN | Dermatopontin OS = Homo sapiens | 1 | 5 | 1.48 |
| | GN = DPT PE = 2 SV = 2 | | | |
| DESM_HUMAN | Desmin OS = Homo sapiens | 1 | 33 | 1.15 |
| | GN = DES PE = 1 SV = 3 | | | |
| DHSB_HUMAN | Succinate dehydrogenase | 1 | 7 | −1.01 |
| | [ubiquinone] iron-sulfur subunit, | | | |
| | mitochondrial OS = Homo sapiens | | | |
| | GN = SDHB PE = 1 SV = 3 | | | |
| DLDH_HUMAN | Dihydrolipoyl dehydrogenase, | 1 | 5 | 1.10 |
| | mitochondrial OS = Homo sapiens | | | |
| | GN = DLD PE = 1 SV = 1 | | | |
| DMC1_HUMAN | Meiotic recombination protein | 0.92 | 1 | −1.17 |
| | DMC1/LIM15 homolog OS = | | | |
| | Homo sapiens GN = DMC1 PE = 1 | | | |
| | SV = 2 | | | |
| DSG2_HUMAN | Desmoglein-2 OS = Homo sapiens | 1 | 4 | 1.08 |
| | GN = DSG2 PE = 1 SV = 2 | | | |
| DYH11_HUMAN | Dynein heavy chain 11, axonemal | 0 | 1 | −1.22 |
| | OS = Homo sapiens GN = DNAH11 | | | |
| | PE = 1 SV = 2 | | | |
| DYXC1_HUMAN | Dyslexia susceptibility 1 | 0 | 1 | −1.15 |
| | candidate gene 1 protein | | | |
| | OS = Homo sapiens GN = DYX1C1 | | | |
| | PE = 2 SV = 2 | | | |
| ECH1_HUMAN | Delta(3,5)-Delta(2,4)-dienoyl-CoA | 0.47 | 1 | 1.25 |
| | isomerase, mitochondrial | | | |
| | OS = Homo sapiens GN = ECH1 | | | |
| | PE = 1 SV = 2 | | | |
| ECHM_HUMAN | Enoyl-CoA hydratase, | 1 | 7 | 1.08 |
| | mitochondrial OS = Homo sapiens | | | |
| | GN = ECHS1 PE = 1 SV = 4 | | | |
| ECT2_HUMAN | Protein ECT2 OS = Homo sapiens | 0 | 1 | 1.01 |
| | GN = ECT2 PE = 1 SV = 3 | | | |
| EF1A2_HUMAN | Elongation factor 1-alpha 2 | 1 | 11 | −1.05 |
| | OS = Homo sapiens GN = EEF1A2 | | | |
| | PE = 1 SV = 1 | | | |
| EF1A3_HUMAN | Putative elongation factor 1- | 1 | 12 | 1.05 |
| | alpha-1ike 3 OS = Homo sapiens | | | |
| | GN = EEF1AL3 PE = 5 SV = 1 | | | |
| EF1B_HUMAN | Elongation factor 1-beta | 0.82 | 1 | −1.13 |
| | OS = Homo sapiens GN = EEF1B2 | | | |
| | PE = 1 SV = 3 | | | |
| EFTU_HUMAN | Elongation factor Tu, | 1 | 6 | 1.06 |
| | mitochondrial OS = Homo sapiens | | | |
| | GN = TUFM PE = 1 SV = 2 | | | |
| ENPL_HUMAN | Endoplasmin OS = Homo sapiens | 1 | 15 | 1.06 |
| | GN = HSP90B1 PE = 1 SV = 1 | | | |
| EPDR1_HUMAN | Mammalian ependymin-related | 0.83 | 1 | −1.72 |
| | protein 1 OS = Homo sapiens | | | |
| | GN = EPDR1 PE = 1 SV = 2 | | | |
| ES1_HUMAN | ES1 protein homolog, | 1 | 15 | −1.09 |
| | mitochondrial OS = Homo sapiens | | | |
| | GN = C21orf33 PE = 1 SV = 3 | | | |
| ETFA_HUMAN | Electron transfer flavoprotein | 1 | 20 | −1.01 |
| | subunit alpha, mitochondrial | | | |
| | OS = Homo sapiens GN = ETFA PE = 1 | | | |
| | SV = 1 | | | |
| F168B_HUMAN | UPF0541 protein FAM168B | 0 | 1 | −1.01 |
| | OS = Homo sapiens GN = FAM168B | | | |
| | PE = 2 SV = 1 | | | |
| FA9_HUMAN | Coagulation factor IX OS = | 1 | 8 | −1.53 |
| | Homo sapiens GN = F9 PE = 1 SV = 2 | | | |
| FABP4_HUMAN | Fatty acid-binding protein, | 0.62 | 1 | 1.04 |
| | adipocyte OS = Homo sapiens | | | |
| | GN = FABP4 PE = 1 SV = 3 | | | |
| FABP5_HUMAN | Fatty acid-binding protein, | 1 | 6 | 1.10 |
| | epidermal OS = Homo sapiens | | | |
| | GN = FABP5 PE = 1 SV = 3 | | | |
| FABPH_HUMAN | Fatty acid-binding protein, heart | 1 | 19 | −1.02 |
| | OS = Homo sapiens GN = FABP3 | | | |
| | PE = 1 SV = 4 | | | |
| FB5L3_HUMAN | Putative fatty acid-binding | 0.69 | 1 | −1.08 |
| | protein 5-like protein 3 OS = | | | |
| | Homo sapiens GN = FABP5L3 PE = 3 | | | |
| | SV = 1 | | | |
| FBLN1_HUMAN | Fibulin-1 OS = Homo sapiens | 0.99 | 2 | 1.13 |
| | GN = FBLN1 PE = 1 SV = 4 | | | |
| FBLN2_HUMAN | Fibulin-2 OS = Homo sapiens | 1 | 4 | 1.71 |
| | GN = FBLN2 PE = 1 SV = 2 | | | |
| FBLN3_HUMAN | EGF-containing fibulin-like | 0.95 | 2 | 1.71 |
| | extracellular matrix protein 1 | | | |
| | OS = Homo sapiens GN = EFEMP1 | | | |
| | PE = 1 SV = 2 | | | |
| FBN1_HUMAN | Fibrillin-1 OS = Homo sapiens | 1 | 48 | 1.79 |
| | GN = FBN1 PE = 1 SV = 2 | | | |
| FETUA_HUMAN | Alpha-2-HS-glycoprotein | 1 | 14 | 1.03 |
| | OS = Homo sapiens GN = AHSG | | | |
| | PE = 1 SV = 1 | | | |
| FHL1_HUMAN | Four and a half LIM domains | 1 | 22 | 1.12 |
| | protein 1 OS = Homo sapiens | | | |
| | GN = FHL1 PE = 1 SV = 4 | | | |
| FHL2_HUMAN | Four and a half LIM domains | 1 | 40 | −1.02 |
| | protein 2 OS = Homo sapiens | | | |
| | GN = FHL2 PE = 1 SV = 3 | | | |
| FIBA_HUMAN | Fibrinogen alpha chain OS = | 1 | 9 | 1.16 |
| | Homo sapiens GN = FGA PE = 1 | | | |
| | SV = 2 | | | |
| FIBB_HUMAN | Fibrinogen beta chain OS = | 1 | 15 | 1.19 |
| | Homo sapiens GN = FGB PE = 1 | | | |
| | SV = 2 | | | |
| FIBG_HUMAN | Fibrinogen gamma chain | 1 | 13 | 1.22 |
| | OS = Homo sapiens GN = FGG PE = 1 | | | |
| | SV = 3 | | | |
| FKBP3_HUMAN | FK506-binding protein 3 | 0.99 | 2 | −1.26 |
| | OS = Homo sapiens GN = FKBP3 | | | |
| | PE = 1 SV = 1 | | | |
| FMOD_HUMAN | Fibromodulin OS = Homo sapiens | 0.97 | 1 | 1.99 |
| | GN = FMOD PE = 1 SV = 2 | | | |
| FRAS1_HUMAN | Extracellular matrix protein | 0.72 | 1 | −1.10 |
| | FRAS1 OS = Homo sapiens | | | |
| | GN = FRAS1 PE = 2 SV = 1 | | | |
| FRIH_HUMAN | Ferritin heavy chain OS = | 1 | 8 | 1.51 |
| | Homo sapiens GN = FTH1 PE = 1 | | | |
| | SV = 2 | | | |
| FSTL4_HUMAN | Follistatin-related protein 4 | 0.36 | 1 | 1.23 |
| | OS = Homo sapiens GN = FSTL4 | | | |
| | PE = 2 SV = 2 | | | |
| FUMH_HUMAN | Fumarate hydratase, | 0.32 | 1 | 1.16 |
| | mitochondrial OS = Homo sapiens | | | |
| | GN = FH PE = 1 SV = 3 | | | |
| G3P_HUMAN | Glyceraldehyde-3-phosphate | 1 | 57 | −1.02 |
| | dehydrogenase OS = Homo sapiens | | | |
| | GN = GAPDH PE = 1 SV = 3 | | | |
| GCSH_HUMAN | Glycine cleavage system H | 0.89 | 1 | −1.37 |
| | protein, mitochondrial OS = | | | |
| | Homo sapiens GN = GCSH PE = 1 | | | |
| | SV = 1 | | | |
| GDIR1_HUMAN | Rho GDP-dissociation inhibitor 1 | 1 | 3 | −1.07 |
| | OS = Homo sapiens GN = ARHGDIA | | | |
| | PE = 1 SV = 3 | | | |
| GDIR2_HUMAN | Rho GDP-dissociation inhibitor 2 | 0 | 1 | −1.02 |
| | OS = Homo sapiens GN = ARHGDIB | | | |
| | PE = 1 SV = 3 | | | |
| GGT2_HUMAN | Gamma-glutamyltranspeptidase | 0.98 | 3 | −1.11 |
| | 2 OS = Homo sapiens GN = GGT2 | | | |
| | PE = 1 SV = 3 | | | |
| GLYG_HUMAN | Glycogenin-1 OS = Homo sapiens | 1 | 13 | −1.28 |
| | GN = GYG1 PE = 1 SV = 4 | | | |
| GPC1_HUMAN | Glypican-1 OS = Homo sapiens | 0.99 | 2 | −1.36 |
| | GN = GPC1 PE = 1 SV = 1 | | | |
| GPNMB_HUMAN | Transmembrane glycoprotein | 1 | 8 | −1.18 |
| | NMB OS = Homo sapiens | | | |
| | GN = GPNMB PE = 1 SV = 2 | | | |
| GRB1L_HUMAN | GREB1-like protein OS = | 0.49 | 1 | −1.24 |
| | Homo sapiens GN = KIAA1772 PE = 2 | | | |
| | SV = 2 | | | |
| GRP75_HUMAN | Stress-70 protein, mitochondrial | 1 | 14 | 1.26 |
| | OS = Homo sapiens GN = HSPA9 | | | |
| | PE = 1 SV = 2 | | | |
| GRP78_HUMAN | 78 kDa glucose-regulated protein | 1 | 6 | 1.10 |
| | OS = Homo sapiens GN = HSPA5 | | | |
| | PE = 1 SV = 2 | | | |
| GSTO1_HUMAN | Glutathione S-transferase omega- | 0.44 | 1 | 1.16 |
| | 1 OS = Homo sapiens GN = GSTO1 | | | |
| | PE = 1 SV = 2 | | | |
| H10_HUMAN | Histone H1.0 OS = Homo sapiens | 0.95 | 2 | −1.19 |
| | GN = H1F0 PE = 1 SV = 3 | | | |
| H11_HUMAN | Histone H1.1 OS = Homo sapiens | 0.58 | 1 | 1.52 |
| | GN = HIST1H1A PE = 1 SV = 3 | | | |
| H12_HUMAN | Histone H1.2 OS = Homo sapiens | 1 | 21 | 1.03 |
| | GN = HIST1H1C PE = 1 SV = 2 | | | |
| H1T_HUMAN | Histone H1t OS = Homo sapiens | 0.98 | 3 | 1.10 |
| | GN = HIST1H1T PE = 1 SV = 4 | | | |
| H2A1B_HUMAN | Histone H2A type 1-B/E | 0.74 | 1 | −1.13 |
| | OS = Homo sapiens | | | |
| | GN = HIST1H2AB PE = 1 SV = 2 | | | |
| H2A2A_HUMAN | Histone H2A type 2-A OS = | 1 | 16 | 1.22 |
| | Homo sapiens GN = HIST2H2AA3 | | | |
| | PE = 1 SV = 3 | | | |
| H2AY_HUMAN | Core histone macro-H2A.1 | 1 | 3 | 1.12 |
| | OS = Homo sapiens GN = H2AFY | | | |
| | PE = 1 SV = 4 | | | |
| H2AZ_HUMAN | Histone H2A.Z OS = Homo sapiens | 0.83 | 1 | 1.36 |
| | GN = H2AFZ PE = 1 SV = 2 | | | |
| H2B1M_HUMAN | Histone H2B type 1-C/E/F/G/I | 1 | 14 | 1.13 |
| | OS = Homo sapiens | | | |
| | GN = HIST1H2BC PE = 1 SV = 3 | | | |
| H2B2C_HUMAN | Putative histone H2B type 2-D | 1 | 4 | 1.05 |
| | OS = Homo sapiens | | | |
| | GN = HIST2H2BD PE = 5 SV = 3 | | | |
| H2B3B_HUMAN | Histone H2B type 3-B OS = | 0.97 | 2 | −1.04 |
| | Homo sapiens GN = HIST3H2BB | | | |
| | PE = 1 SV = 3 | | | |
| H31_HUMAN | Histone H3.1 OS = Homo sapiens | 1 | 1 | 1.21 |
| | GN = HIST1H3A PE = 1 SV = 2 | | | |
| H31T_HUMAN | Histone H3.1t OS = Homo sapiens | 0.62 | 1 | −1.14 |
| | GN = HIST3H3 PE = 1 SV = 3 | | | |
| H32_HUMAN | Histone H3.2 OS = Homo sapiens | 1 | 1 | 1.06 |
| | GN = HIST2H3A PE = 1 SV = 3 | | | |
| H33_HUMAN | Histone H3.3 OS = Homo sapiens | 1 | 9 | 1.09 |
| | GN = H3F3A PE = 1 SV = 2 | | | |
| H3L_HUMAN | Histone H3-like OS = Homo sapiens | 0.67 | 1 | −1.57 |
| | PE = 2 SV = 3 | | | |
| H4_HUMAN | Histone H4 OS = Homo sapiens | 1 | 10 | 1.30 |
| | GN = HIST1H4A PE = 1 SV = 2 | | | |
| HBA_HUMAN | Hemoglobin subunit alpha | 1 | 12 | −1.19 |
| | OS = Homo sapiens GN = HBA1 | | | |
| | PE = 1 SV = 2 | | | |
| HBB_HUMAN | Hemoglobin subunit beta | 1 | 12 | −1.38 |
| | OS = Homo sapiens GN = HBB | | | |
| | PE = 1 SV = 2 | | | |
| HBD_HUMAN | Hemoglobin subunit delta | 0.92 | 1 | 1.08 |
| | OS = Homo sapiens GN = HBD | | | |
| | PE = 1 SV = 2 | | | |
| HBE_HUMAN | Hemoglobin subunit epsilon | 0.72 | 1 | 1.16 |
| | OS = Homo sapiens GN = HBE1 | | | |
| | PE = 1 SV = 2 | | | |
| HBG1_HUMAN | Hemoglobin subunit gamma-1 | 0.93 | 2 | 1.01 |
| | OS = Homo sapiens GN = HBG1 | | | |
| | PE = 1 SV = 2 | | | |
| HCDH_HUMAN | Hydroxyacyl-coenzyme A | 1 | 4 | 1.56 |
| | dehydrogenase, mitochondrial | | | |
| | OS = Homo sapiens GN = HADH | | | |
| | PE = 1 SV = 2 | | | |
| HEBP2_HUMAN | Heme-binding protein 2 | 0.38 | 1 | 1.12 |
| | OS = Homo sapiens GN = HEBP2 | | | |
| | PE = 1 SV = 1 | | | |
| HEMO_HUMAN | Hemopexin OS = Homo sapiens | 1 | 30 | −1.26 |
| | GN = HPX PE = 1 SV = 2 | | | |
| HMGB2_HUMAN | High mobility group protein B2 | 0.98 | 2 | −1.17 |
| | OS = Homo sapiens GN = HMGB2 | | | |
| | PE = 1 SV = 2 | | | |
| HNRPC_HUMAN | Heterogeneous nuclear | 1 | 3 | −1.15 |
| | ribonucleoproteins C1/C2 | | | |
| | OS = Homo sapiens GN = HNRNPC | | | |
| | PE = 1 SV = 4 | | | |
| HNRPD_HUMAN | Heterogeneous nuclear | 1 | 5 | 1.07 |
| | ribonucleoprotein D0 OS = | | | |
| | Homo sapiens GN = HNRNPD PE = 1 | | | |
| | SV = 1 | | | |
| HP1B3_HUMAN | Heterochromatin protein 1- | 1 | 4 | −1.03 |
| | binding protein 3 OS = | | | |
| | Homo sapiens GN = HP1BP3 PE = 1 | | | |
| | SV = 1 | | | |
| HPT_HUMAN | Haptoglobin OS = Homo sapiens | 1 | 43 | −1.21 |
| | GN = HP PE = 1 SV = 1 | | | |
| HRG_HUMAN | Histidine-rich glycoprotein | 0.99 | 3 | −1.06 |
| | OS = Homo sapiens GN = HRG PE = 1 | | | |
| | SV = 1 | | | |
| HS90A_HUMAN | Heat shock protein HSP 90-alpha | 1 | 16 | 1.05 |
| | OS = Homo sapiens GN = HSP90AA1 | | | |
| | PE = 1 SV = 5 | | | |
| HS90B_HUMAN | Heat shock protein HSP 90-beta | 1 | 3 | 1.06 |
| | OS = Homo sapiens GN = HSP90AB1 | | | |
| | PE = 1 SV = 4 | | | |
| HSP76_HUMAN | Heat shock 70 kDa protein 6 | 0 | 1 | 1.07 |
| | OS = Homo sapiens GN = HSPA6 | | | |
| | PE = 1 SV = 2 | | | |
| HSPB1_HUMAN | Heat shock protein beta-1 | 1 | 24 | −1.10 |
| | OS = Homo sapiens GN = HSPB1 | | | |
| | PE = 1 SV = 2 | | | |
| HSPB2_HUMAN | Heat shock protein beta-2 | 0.94 | 1 | −1.06 |
| | OS = Homo sapiens GN = HSPB2 | | | |
| | PE = 1 SV = 2 | | | |
| HSPB7_HUMAN | Heat shock protein beta-7 | 1 | 13 | −1.15 |
| | OS = Homo sapiens GN = HSPB7 | | | |
| | PE = 1 SV = 1 | | | |
| IC1_HUMAN | Plasma protease C1 inhibitor | 1 | 10 | −1.22 |
| | OS = Homo sapiens GN = SERPING1 | | | |
| | PE = 1 SV = 2 | | | |
| ICAL_HUMAN | Calpastatin OS = Homo sapiens | 1 | 20 | −1.40 |
| | GN = CAST PE = 1 SV = 4 | | | |
| IDHP_HUMAN | Isocitrate dehydrogenase [NADP], | 1 | 35 | −1.07 |
| | mitochondrial OS = Homo sapiens | | | |
| | GN = IDH2 PE = 1 SV = 2 | | | |
| IF4H_HUMAN | Eukaryotic translation initiation | 0.94 | 1 | 1.00 |
| | factor 4H OS = Homo sapiens | | | |
| | GN = EIF4H PE = 1 SV = 5 | | | |
| IF5A1_HUMAN | Eukaryotic translation initiation | 1 | 9 | −1.09 |
| | factor 5A-1 OS = Homo sapiens | | | |
| | GN = EIF5A PE = 1 SV = 2 | | | |
| IGHA1_HUMAN | Ig alpha-1 chain C region | 1 | 17 | −1.08 |
| | OS = Homo sapiens GN = IGHA1 | | | |
| | PE = 1 SV = 2 | | | |
| IGHA2_HUMAN | Ig alpha-2 chain C region | 1 | 4 | −1.64 |
| | OS = Homo sapiens GN = IGHA2 | | | |
| | PE = 1 SV = 3 | | | |
| IGHG1_HUMAN | Ig gamma-1 chain C region | 1 | 16 | 1.21 |
| | OS = Homo sapiens GN = IGHG1 | | | |
| | PE = 1 SV = 1 | | | |
| IGHG2_HUMAN | Ig gamma-2 chain C region | 1 | 28 | 1.24 |
| | OS = Homo sapiens GN = IGHG2 | | | |
| | PE = 1 SV = 2 | | | |
| IGHG3_HUMAN | Ig gamma-3 chain C region | 1 | 4 | −1.19 |
| | OS = Homo sapiens GN = IGHG3 | | | |
| | PE = 1 SV = 2 | | | |
| IGHG4_HUMAN | Ig gamma-4 chain C region | 1 | 6 | 1.61 |
| | OS = Homo sapiens GN = IGHG4 | | | |
| | PE = 1 SV = 1 | | | |
| IGHM_HUMAN | Ig mu chain C region OS = | 0.99 | 2 | −1.08 |
| | Homo sapiens GN = IGHM PE = 1 | | | |
| | SV = 3 | | | |
| IGKC_HUMAN | Ig kappa chain C region OS = | 1 | 19 | 1.05 |
| | Homo sapiens GN = IGKC PE = 1 | | | |
| | SV = 1 | | | |
| IPYR2_HUMAN | Inorganic pyrophosphatase 2, | 0.74 | 2 | −1.26 |
| | mitochondrial OS = Homo sapiens | | | |
| | GN = PPA2 PE = 1 SV = 2 | | | |
| ITB1_HUMAN | Integrin beta-1 OS = Homo sapiens | 1 | 18 | −1.03 |
| | GN = ITGB1 PE = 1 SV = 2 | | | |
| ITIH1_HUMAN | Inter-alpha-trypsin inhibitor | 0.81 | 1 | −1.64 |
| | heavy chain H1 OS = Homo sapiens | | | |
| | GN = ITIH1 PE = 1 SV = 3 | | | |
| ITPR3_HUMAN | Inositol 1,4,5-trisphosphate | 0 | 1 | 1.09 |
| | receptor type 3 OS = Homo sapiens | | | |
| | GN = ITPR3 PE = 1 SV = 2 | | | |
| K0406_HUMAN | Uncharacterized protein | 0.6 | 1 | −1.34 |
| | KIAA0406 OS = Homo sapiens | | | |
| | GN = KIAA0406 PE = 1 SV = 3 | | | |
| KAD1_HUMAN | Adenylate kinase isoenzyme 1 | 1 | 19 | 1.12 |
| | OS = Homo sapiens GN = AK1 PE = 1 | | | |
| | SV = 3 | | | |
| KCRB_HUMAN | Creatine kinase B-type OS = | 1 | 17 | −1.13 |
| | Homo sapiens GN = CKB PE = 1 | | | |
| | SV = 1 | | | |
| KCRM_HUMAN | Creatine kinase M-type OS = | 1 | 41 | −1.23 |
| | Homo sapiens GN = CKM PE = 1 | | | |
| | SV = 2 | | | |
| KCRS_HUMAN | Creatine kinase S-type, | 1 | 22 | 1.03 |
| | mitochondrial OS = Homo sapiens | | | |
| | GN = CKMT2 PE = 1 SV = 2 | | | |
| KCY_HUMAN | UMP-CMP kinase OS = | 0.99 | 3 | 1.04 |
| | Homo sapiens GN = CMPK1 PE = 1 | | | |
| | SV = 3 | | | |
| KLOTB_HUMAN | Beta-klotho OS = Homo sapiens | 0 | 1 | 1.11 |
| | GN = KLB PE = 2 SV = 1 | | | |
| KNG1_HUMAN | Kininogen-1 OS = Homo sapiens | 1 | 10 | −1.16 |
| | GN = KNG1 PE = 1 SV = 2 | | | |
| LAC_HUMAN | Ig lambda chain C regions | 1 | 9 | 1.18 |
| | OS = Homo sapiens GN = IGLC1 | | | |
| | PE = 1 SV = 1 | | | |
| LAMA2_HUMAN | Laminin subunit alpha-2 | 1 | 77 | −1.27 |
| | OS = Homo sapiens GN = LAMA2 | | | |
| | PE = 1 SV = 4 | | | |
| LAMA4_HUMAN | Laminin subunit alpha-4 | 1 | 11 | −1.07 |
| | OS = Homo sapiens GN = LAMA4 | | | |
| | PE = 1 SV = 3 | | | |
| LAMB1_HUMAN | Laminin subunit beta-1 OS = | 1 | 28 | −1.28 |
| | Homo sapiens GN = LAMB1 PE = 1 | | | |
| | SV = 1 | | | |
| LAMB2_HUMAN | Laminin subunit beta-2 OS = | 1 | 39 | −1.03 |
| | Homo sapiens GN = LAMB2 PE = 1 | | | |
| | SV = 2 | | | |
| LAMC1_HUMAN | Laminin subunit gamma-1 | 1 | 57 | −1.14 |
| | OS = Homo sapiens GN = LAMC1 | | | |
| | PE = 1 SV = 3 | | | |
| LAMP1_HUMAN | Lysosome-associated membrane | 0.78 | 1 | −1.40 |
| | glycoprotein 1 OS = Homo sapiens | | | |
| | GN = LAMP1 PE = 1 SV = 3 | | | |
| LCORL_HUMAN | Ligand-dependent nuclear | 0.28 | 1 | −1.09 |
| | receptor corepressor-like protein | | | |
| | OS = Homo sapiens GN = LCORL | | | |
| | PE = 2 SV = 4 | | | |
| LDB3_HUMAN | LIM domain-binding protein 3 | 1 | 4 | −1.03 |
| | OS = Homo sapiens GN = LDB3 PE = 1 | | | |
| | SV = 2 | | | |
| LDHB_HUMAN | L-lactate dehydrogenase B chain | 1 | 19 | −1.08 |
| | OS = Homo sapiens GN = LDHB PE = 1 | | | |
| | SV = 2 | | | |
| LEG1_HUMAN | Galectin-1 OS = Homo sapiens | 1 | 17 | −1.09 |
| | GN = LGALS1 PE = 1 SV = 2 | | | |
| LG3BP_HUMAN | Galectin-3-binding protein | 1 | 17 | −1.47 |
| | OS = Homo sapiens GN = LGALS3BP | | | |
| | PE = 1 SV = 1 | | | |
| LTBP2_HUMAN | Latent-transforming growth | 1 | 6 | 1.44 |
| | factor beta-binding protein 2 | | | |
| | OS = Homo sapiens GN = LTBP2 | | | |
| | PE = 1 SV = 2 | | | |
| LU_HUMAN | Lutheran blood group glycoprotein | 1 | 7 | −1.07 |
| | precursor - Homo sapiens | | | |
| LUM_HUMAN | Lumican OS = Homo sapiens | 1 | 31 | 1.11 |
| | GN = LUM PE = 1 SV = 2 | | | |
| LV202_HUMAN | Ig lambda chain V-II region NEI | 0.68 | 1 | 1.17 |
| | OS = Homo sapiens PE = 1 SV = 1 | | | |
| LYSC_HUMAN | Lysozyme C OS = Homo sapiens | 0 | 1 | −1.10 |
| | GN = LYZ PE = 1 SV = 1 | | | |
| M3K5_HUMAN | Mitogen-activated protein kinase | 0 | 1 | −1.02 |
| | kinase kinase 5 OS = Homo sapiens | | | |
| | GN = MAP3K5 PE = 1 SV = 1 | | | |
| M6PBP_HUMAN | Mannose-6-phosphate receptor- | 0.46 | 1 | −1.09 |
| | binding protein 1 OS = Homo sapiens | | | |
| | GN = M6PRBP1 PE = 1 SV = 2 | | | |
| MAOM_HUMAN | NAD-dependent malic enzyme, | 0 | 1 | 1.01 |
| | mitochondrial OS = Homo sapiens | | | |
| | GN = ME2 PE = 1 SV = 1 | | | |
| MARCS_HUMAN | Myristoylated alanine-rich C- | 1 | 3 | −1.16 |
| | kinase substrate OS = | | | |
| | Homo sapiens GN = MARCKS PE = 1 | | | |
| | SV = 4 | | | |
| MDHC_HUMAN | Malate dehydrogenase, | 1 | 14 | 1.11 |
| | cytoplasmic OS = Homo sapiens | | | |
| | GN = MDH1 PE = 1 SV = 4 | | | |
| MDHM_HUMAN | Malate dehydrogenase, | 1 | 20 | 1.13 |
| | mitochondrial OS = Homo sapiens | | | |
| | GN = MDH2 PE = 1 SV = 3 | | | |
| MFAP4_HUMAN | Microfibril-associated | 1 | 5 | 1.54 |
| | glycoprotein 4 OS = Homo sapiens | | | |
| | GN = MFAP4 PE = 1 SV = 2 | | | |
| MFAP5_HUMAN | Microfibrillar-associated protein | 1 | 4 | 2.01 |
| | 5 OS = Homo sapiens GN = MFAP5 | | | |
| | PE = 2 SV = 1 | | | |
| MFGM_HUMAN | Lactadherin OS = Homo sapiens | 1 | 14 | −1.06 |
| | GN = MFGE8 PE = 1 SV = 2 | | | |
| MGST3_HUMAN | Microsomal glutathione S- | 0.83 | 1 | 1.93 |
| | transferase 3 OS = Homo sapiens | | | |
| | GN = MGST3 PE = 1 SV = 1 | | | |
| MIME_HUMAN | Mimecan OS = Homo sapiens | 1 | 26 | 1.08 |
| | GN = OGN PE = 1 SV = 1 | | | |
| MLE3_HUMAN | Myosin light chain 3, skeletal | 1 | 5 | −1.14 |
| | muscle isoform OS = | | | |
| | Homo sapiens GN = MYL1 PE = 2 | | | |
| | SV = 2 | | | |
| MLL2_HUMAN | Histone-lysine N- | 0 | 1 | 1.42 |
| | methyltransferase MLL2 | | | |
| | OS = Homo sapiens GN = MLL2 | | | |
| | PE = 1 SV = 1 | | | |
| MLRS_HUMAN | Myosin regulatory light chain 2, | 0.5 | 1 | −1.04 |
| | skeletal muscle isoform | | | |
| | OS = Homo sapiens GN = MYLPF | | | |
| | PE = 2 SV = 1 | | | |
| MLRV_HUMAN | Myosin regulatory light chain 2, | 1 | 43 | 1.11 |
| | ventricular/cardiac muscle | | | |
| | isoform OS = Homo sapiens | | | |
| | GN = MYL2 PE = 1 SV = 3 | | | |
| MPCP_HUMAN | Phosphate carrier protein, | 1 | 5 | 1.04 |
| | mitochondrial OS = Homo sapiens | | | |
| | GN = SLC25A3 PE = 1 SV = 2 | | | |
| MRLC2_HUMAN | Myosin regulatory light chain | 0.93 | 2 | 1.01 |
| | MRLC2 OS = Homo sapiens | | | |
| | GN = MYLC2B PE = 1 SV = 2 | | | |
| MRLC3_HUMAN | Myosin regulatory light chain | 0.66 | 1 | −2.06 |
| | MRLC3 OS = Homo sapiens | | | |
| | GN = MRLC3 PE = 1 SV = 2 | | | |
| MSRB2_HUMAN | Methionine-R-sulfoxide reductase | 0 | 1 | −1.22 |
| | B2, mitochondrial OS = | | | |
| | Homo sapiens GN = MSRB2 PE = 2 | | | |
| | SV = 2 | | | |
| MUC18_HUMAN | Cell surface glycoprotein MUC18 | 1 | 2 | −1.30 |
| | OS = Homo sapiens GN = MCAM | | | |
| | PE = 1 SV = 2 | | | |
| MYG_HUMAN | Myoglobin OS = Homo sapiens | 1 | 27 | 1.19 |
| | GN = MB PE = 1 SV = 2 | | | |
| MYH13_HUMAN | Myosin-13 OS = Homo sapiens | 0.59 | 1 | −1.27 |
| | GN = MYH13 PE = 1 SV = 1 | | | |
| MYH2_HUMAN | Myosin-2 OS = Homo sapiens | 0.95 | 2 | −1.21 |
| | GN = MYH2 PE = 1 SV = 1 | | | |
| MYH3_HUMAN | Myosin-3 OS = Homo sapiens | 0.41 | 1 | −1.17 |
| | GN = MYH3 PE = 1 SV = 3 | | | |
| MYH7_HUMAN | Myosin-7 OS = Homo sapiens | 1 | 66 | 1.18 |
| | GN = MYH7 PE = 1 SV = 5 | | | |
| MYH8_HUMAN | Myosin-8 OS = Homo sapiens | 0.95 | 1 | 1.24 |
| | GN = MYH8 PE = 1 SV = 3 | | | |
| MYL3_HUMAN | Myosin light chain 3 OS = | 1 | 56 | 1.12 |
| | Homo sapiens GN = MYL3 PE = 1 | | | |
| | SV = 3 | | | |
| MYL4_HUMAN | Myosin light chain 4 OS = | 1 | 8 | 1.05 |
| | Homo sapiens GN = MYL4 PE = 1 | | | |
| | SV = 3 | | | |
| MYL6_HUMAN | Myosin light polypeptide 6 | 1 | 5 | 1.32 |
| | OS = Homo sapiens GN = MYL6 | | | |
| | PE = 1 SV = 2 | | | |
| MYL9_HUMAN | Myosin regulatory light | 1 | 12 | 1.05 |
| | polypeptide 9 OS = Homo sapiens | | | |
| | GN = MYL9 PE = 1 SV = 4 | | | |
| MYLPL_HUMAN | Myosin light chain 2, lymphocyte- | 1 | 14 | 1.08 |
| | specific OS = Homo sapiens | | | |
| | GN = MYLC2PL PE = 2 SV = 2 | | | |
| MYO6_HUMAN | Myosin-VI OS = Homo sapiens | 0 | 1 | 1.29 |
| | GN = MYO6 PE = 1 SV = 4 | | | |
| MYOZ2_HUMAN | Myozenin-2 OS = Homo sapiens | 1 | 38 | 1.02 |
| | GN = MYOZ2 PE = 1 SV = 1 | | | |
| MYP2_HUMAN | Myelin P2 protein OS = | 0.89 | 2 | 1.27 |
| | Homo sapiens GN = PMP2 PE = 1 | | | |
| | SV = 3 | | | |
| MYPT1_HUMAN | Protein phosphatase 1 regulatory | 0 | 1 | −1.28 |
| | subunit 12A OS = Homo sapiens | | | |
| | GN = PPP1R12A PE = 1 SV = 1 | | | |
| MYPT2_HUMAN | Protein phosphatase 1 regulatory | 1 | 4 | −1.13 |
| | subunit 12B OS = Homo sapiens | | | |
| | GN = PPP1R12B PE = 1 SV = 2 | | | |
| NCAM1_HUMAN | Neural cell adhesion molecule 1 | 1 | 3 | −1.22 |
| | OS = Homo sapiens GN = NCAM1 | | | |
| | PE = 1 SV = 3 | | | |
| NDKB_HUMAN | Nucleoside diphosphate kinase B | 0.82 | 1 | −1.04 |
| | OS = Homo sapiens GN = NME2 | | | |
| | PE = 1 SV = 1 | | | |
| NDUA4_HUMAN | NADH dehydrogenase | 1 | 2 | −1.06 |
| | [ubiquinone] 1 alpha subcomplex | | | |
| | subunit 4 OS = Homo sapiens | | | |
| | GN = NDUFA4 PE = 1 SV = 1 | | | |
| NDUA7_HUMAN | NADH dehydrogenase | 0.83 | 1 | 1.17 |
| | [ubiquinone] 1 alpha subcomplex | | | |
| | subunit 7 OS = Homo sapiens | | | |
| | GN = NDUFA7 PE = 1 SV = 3 | | | |
| NDUA8_HUMAN | NADH dehydrogenase | 1 | 4 | −1.03 |
| | [ubiquinone] 1 alpha subcomplex | | | |
| | subunit 8 OS = Homo sapiens | | | |
| | GN = NDUFA8 PE = 1 SV = 3 | | | |
| NDUAA_HUMAN | NADH dehydrogenase | 1 | 3 | 1.03 |
| | [ubiquinone] 1 alpha subcomplex | | | |
| | subunit 10, mitochondrial | | | |
| | OS = Homo sapiens GN = NDUFA10 | | | |
| | PE = 1 SV = 1 | | | |
| NDUAC_HUMAN | NADH dehydrogenase | 0.99 | 2 | −1.06 |
| | [ubiquinone] 1 alpha subcomplex | | | |
| | subunit 12 OS = Homo sapiens | | | |
| | GN = NDUFA12 PE = 1 SV = 1 | | | |
| NDUAD_HUMAN | NADH dehydrogenase | 0.75 | 1 | 1.42 |
| | [ubiquinone] 1 alpha subcomplex | | | |
| | subunit 13 OS = Homo sapiens | | | |
| | GN = NDUFA13 PE = 1 SV = 3 | | | |
| NDUB3_HUMAN | NADH dehydrogenase | 0.83 | 1 | −1.11 |
| | [ubiquinone] 1 beta subcomplex | | | |
| | subunit 3 OS = Homo sapiens | | | |
| | GN = NDUFB3 PE = 1 SV = 3 | | | |
| NDUB9_HUMAN | NADH dehydrogenase | 1 | 3 | −1.05 |
| | [ubiquinone] 1 beta subcomplex | | | |
| | subunit 9 OS = Homo sapiens | | | |
| | GN = NDUFB9 PE = 1 SV = 3 | | | |
| NDUBA_HUMAN | NADH dehydrogenase | 1 | 16 | −1.00 |
| | [ubiquinone] 1 beta subcomplex | | | |
| | subunit 10 OS = Homo sapiens | | | |
| | GN = NDUFB10 PE = 1 SV = 3 | | | |
| NDUS4_HUMAN | NADH dehydrogenase | 1 | 16 | −1.21 |
| | [ubiquinone] iron-sulfur protein | | | |
| | 4, mitochondrial OS = | | | |
| | Homo sapiens GN = NDUFS4 PE = 1 | | | |
| | SV = 1 | | | |
| NDUS5_HUMAN | NADH dehydrogenase | 0.99 | 2 | 1.12 |
| | [ubiquinone] iron-sulfur protein 5 | | | |
| | OS = Homo sapiens GN = NDUFS5 | | | |
| | PE = 1 SV = 3 | | | |
| NDUS6_HUMAN | NADH dehydrogenase | 1 | 15 | −1.19 |
| | [ubiquinone] iron-sulfur protein | | | |
| | 6, mitochondrial OS = | | | |
| | Homo sapiens GN = NDUFS6 PE = 1 | | | |
| | SV = 1 | | | |
| NDUS7_HUMAN | NADH dehydrogenase | 1 | 7 | −1.07 |
| | [ubiquinone] iron-sulfur protein | | | |
| | 7, mitochondrial OS = | | | |
| | Homo sapiens GN = NDUFS7 PE = 1 | | | |
| | SV = 3 | | | |
| NDUS8_HUMAN | NADH dehydrogenase | 0.61 | 1 | 1.07 |
| | [ubiquinone] iron-sulfur protein | | | |
| | 8, mitochondrial OS = | | | |
| | Homo sapiens GN = NDUFS8 PE = 1 | | | |
| | SV = 1 | | | |
| NDUV1_HUMAN | NADH dehydrogenase | 1 | 5 | −1.06 |
| | [ubiquinone] flavoprotein 1, | | | |
| | mitochondrial OS = Homo sapiens | | | |
| | GN = NDUFV1 PE = 1 SV = 4 | | | |
| NDUV2_HUMAN | NADH dehydrogenase | 0.97 | 2 | 1.07 |
| | [ubiquinone] flavoprotein 2, | | | |
| | mitochondrial OS = Homo sapiens | | | |
| | GN = NDUFV2 PE = 1 SV = 2 | | | |
| NDUV3_HUMAN | NADH dehydrogenase | 0.83 | 1 | −1.05 |
| | [ubiquinone] flavoprotein 3, | | | |
| | mitochondrial OS = Homo sapiens | | | |
| | GN = NDUFV3 PE = 2 SV = 2 | | | |
| NEBL_HUMAN | Nebulette OS = Homo sapiens | 1 | 7 | 1.30 |
| | GN = NEBL PE = 1 SV = 1 | | | |
| NEXN_HUMAN | Nexilin OS = Homo sapiens | 0.98 | 2 | 1.19 |
| | GN = NEXN PE = 1 SV = 1 | | | |
| NID1_HUMAN | Nidogen-1 OS = Homo sapiens | 0.3 | 1 | −1.02 |
| | GN = NID1 PE = 1 SV = 3 | | | |
| NID2_HUMAN | Nidogen-2 OS = Homo sapiens | 1 | 29 | −1.01 |
| | GN = NID2 PE = 1 SV = 2 | | | |
| NP1L1_HUMAN | Nucleosome assembly protein 1- | 0.43 | 1 | −1.06 |
| | like 1 OS = Homo sapiens | | | |
| | GN = NAP1L1 PE = 1 SV = 1 | | | |
| NP1L4_HUMAN | Nucleosome assembly protein 1- | 0.98 | 2 | −1.06 |
| | like 4 OS = Homo sapiens | | | |
| | GN = NAP1L4 PE = 1 SV = 1 | | | |
| NPM_HUMAN | Nucleophosmin OS = | 1 | 6 | −1.07 |
| | Homo sapiens GN = NPM1 PE = 1 | | | |
| | SV = 2 | | | |
| NUCL_HUMAN | Nucleolin OS = Homo sapiens | 0.99 | 4 | −1.00 |
| | GN = NCL PE = 1 SV = 3 | | | |
| OBFC1_HUMAN | Oligonucleotide/oligosaccharide- | 0 | 1 | −1.13 |
| | binding fold-containing protein 1 | | | |
| | OS = Homo sapiens GN = OBFC1 | | | |
| | PE = 2 SV = 2 | | | |
| OCAD1_HUMAN | OCIA domain-containing protein | 1 | 6 | −1.02 |
| | 1 OS = Homo sapiens GN = OCIAD1 | | | |
| | PE = 1 SV = 1 | | | |
| ODPA_HUMAN | Pyruvate dehydrogenase E1 | 1 | 11 | 1.05 |
| | component subunit alpha, | | | |
| | somatic form, mitochondrial | | | |
| | OS = Homo sapiens GN = PDHA1 | | | |
| | PE = 1 SV = 3 | | | |
| PABP3_HUMAN | Polyadenylate-binding protein 3 | 0 | 1 | 1.14 |
| | OS = Homo sapiens GN = PABPC3 | | | |
| | PE = 1 SV = 2 | | | |
| PAL4B_HUMAN | Peptidylprolyl cis-trans isomerase | 1 | 9 | −1.10 |
| | A-like 4B OS = Homo sapiens | | | |
| | GN = PPIAL4B PE = 1 SV = 1 | | | |
| PARC_HUMAN | p53-associated parkin-like | 0 | 1 | −1.07 |
| | cytoplasmic protein - Homo sapiens | | | |
| PARK7_HUMAN | Protein DJ-1 OS = Homo sapiens | 1 | 3 | 1.16 |
| | GN = PARK7 PE = 1 SV = 2 | | | |
| PCDH9_HUMAN | Protocadherin-9 OS = | 0 | 1 | 1.07 |
| | Homo sapiens GN = PCDH9 PE = 1 | | | |
| | SV = 2 | | | |
| PDIA1_HUMAN | Protein disulfide-isomerase | 0.83 | 1 | −1.07 |
| | OS = Homo sapiens GN = P4HB | | | |
| | PE = 1 SV = 3 | | | |
| PDIA3_HUMAN | Protein disulfide-isomerase A3 | 1 | 5 | −1.01 |
| | OS = Homo sapiens GN = PDIA3 | | | |
| | PE = 1 SV = 4 | | | |
| PDIA6_HUMAN | Protein disulfide-isomerase A6 | 0.81 | 1 | 1.02 |
| | OS = Homo sapiens GN = PDIA6 | | | |
| | PE = 1 SV = 1 | | | |
| PDLI1_HUMAN | PDZ and LIM domain protein 1 | 1 | 9 | 1.17 |
| | OS = Homo sapiens GN = PDLIM1 | | | |
| | PE = 1 SV = 4 | | | |
| PDLI3_HUMAN | PDZ and LIM domain protein 3 | 1 | 4 | 1.44 |
| | OS = Homo sapiens GN = PDLIM3 | | | |
| | PE = 2 SV = 1 | | | |
| PDLI5_HUMAN | PDZ and LIM domain protein 5 | 1 | 16 | −1.03 |
| | OS = Homo sapiens GN = PDLIM5 | | | |
| | PE = 1 SV = 4 | | | |
| PEBP1_HUMAN | Phosphatidylethanolamine- | 1 | 16 | −1.08 |
| | binding protein 1 OS = | | | |
| | Homo sapiens GN = PEBP1 PE = 1 | | | |
| | SV = 3 | | | |
| PGAM1_HUMAN | Phosphoglycerate mutase 1 | 1 | 9 | −1.09 |
| | OS = Homo sapiens GN = PGAM1 | | | |
| | PE = 1 SV = 2 | | | |
| PGAM2_HUMAN | Phosphoglycerate mutase 2 | 1 | 11 | 1.15 |
| | OS = Homo sapiens GN = PGAM2 | | | |
| | PE = 1 SV = 3 | | | |
| PGBM_HUMAN | Basement membrane-specific | 1 | 21 | 1.16 |
| | heparan sulfate proteoglycan | | | |
| | core protein OS = Homo sapiens | | | |
| | GN = HSPG2 PE = 1 SV = 3 | | | |
| PGK1_HUMAN | Phosphoglycerate kinase 1 | 0.99 | 3 | 1.20 |
| | OS = Homo sapiens GN = PGK1 | | | |
| | PE = 1 SV = 3 | | | |
| PGRC2_HUMAN | Membrane-associated | 0 | 1 | −1.07 |
| | progesterone receptor | | | |
| | component 2 OS = Homo sapiens | | | |
| | GN = PGRMC2 PE = 1 SV = 1 | | | |
| PGS1_HUMAN | Biglycan OS = Homo sapiens | 1 | 23 | 1.25 |
| | GN = BGN PE = 1 SV = 2 | | | |
| PGS2_HUMAN | Decorin OS = Homo sapiens | 1 | 26 | 1.09 |
| | GN = DCN PE = 1 SV = 1 | | | |
| PHP14_HUMAN | 14 kDa phosphohistidine | 0 | 1 | −1.25 |
| | phosphatase OS = Homo sapiens | | | |
| | GN = PHPT1 PE = 1 SV = 1 | | | |
| PLCF_HUMAN | 1-acyl-sn-glycerol-3-phosphate | 0 | 1 | 1.02 |
| | acyltransferase zeta OS = | | | |
| | Homo sapiens GN = AGPAT6 PE = 1 | | | |
| | SV = 1 | | | |
| POPD1_HUMAN | Blood vessel epicardial substance | 0.94 | 1 | −1.20 |
| | OS = Homo sapiens GN = BVES PE = 2 | | | |
| | SV = 1 | | | |
| PPIA_HUMAN | Peptidyl-prolyl cis-trans | 1 | 18 | −1.06 |
| | isomerase A OS = Homo sapiens | | | |
| | GN = PPIA PE = 1 SV = 2 | | | |
| PPIF_HUMAN | Peptidyl-prolyl cis-trans | 1 | 12 | −1.49 |
| | isomerase, mitochondrial | | | |
| | OS = Homo sapiens GN = PPIF | | | |
| | PE = 1 SV = 1 | | | |
| PRDX1_HUMAN | Peroxiredoxin-1 OS = | 1 | 3 | 1.05 |
| | Homo sapiens GN = PRDX1 PE = 1 | | | |
| | SV = 1 | | | |
| PRDX2_HUMAN | Peroxiredoxin-2 OS = | 1 | 9 | −1.02 |
| | Homo sapiens GN = PRDX2 PE = 1 | | | |
| | SV = 5 | | | |
| PRDX3_HUMAN | Thioredoxin-dependent peroxide | 1 | 6 | 1.08 |
| | reductase, mitochondrial | | | |
| | OS = Homo sapiens GN = PRDX3 | | | |
| | PE = 1 SV = 3 | | | |
| PRELP_HUMAN | Prolargin OS = Homo sapiens | 1 | 28 | 1.31 |
| | GN = PRELP PE = 1 SV = 1 | | | |
| PROF1_HUMAN | Profilin-1 OS = Homo sapiens | 1 | 3 | 1.15 |
| | GN = PFN1 PE = 1 SV = 2 | | | |
| PSD7_HUMAN | 26S proteasome non-ATPase | 0 | 1 | 1.03 |
| | regulatory subunit 7 OS = | | | |
| | Homo sapiens GN = PSMD7 PE = 1 | | | |
| | SV = 2 | | | |
| PTGDS_HUMAN | Prostaglandin-H2 D-isomerase | 1 | 6 | −1.53 |
| | OS = Homo sapiens GN = PTGDS | | | |
| | PE = 1 SV = 1 | | | |
| PTN11_HUMAN | Tyrosine-protein phosphatase | 0.38 | 1 | −1.13 |
| | non-receptor type 11 OS = | | | |
| | Homo sapiens GN = PTPN11 PE = 1 | | | |
| | SV = 2 | | | |
| PTRF_HUMAN | Polymerase I and transcript | 1 | 19 | 1.07 |
| | release factor OS = Homo sapiens | | | |
| | GN = PTRF PE = 1 SV = 1 | | | |
| PURA_HUMAN | Transcriptional activator protein | 0.99 | 1 | −1.01 |
| | Pur-alpha OS = Homo sapiens | | | |
| | GN = PURA PE = 1 SV = 2 | | | |
| QCR6_HUMAN | Cytochrome b-c1 complex | 1 | 11 | −1.17 |
| | subunit 6, mitochondrial | | | |
| | OS = Homo sapiens GN = UQCRH | | | |
| | PE = 1 SV = 2 | | | |
| QCR7_HUMAN | Cytochrome b-c1 complex | 0.82 | 1 | 1.01 |
| | subunit 7 OS = Homo sapiens | | | |
| | GN = UQCRB PE = 1 SV = 2 | | | |
| QIL1_HUMAN | Protein QIL1 OS = Homo sapiens | 0.97 | 3 | −1.07 |
| | GN = QIL1 PE = 1 SV = 1 | | | |
| RABE2_HUMAN | Rab GTPase-binding effector | 0.38 | 1 | −1.04 |
| | protein 2 OS = Homo sapiens | | | |
| | GN = RABEP2 PE = 1 SV = 2 | | | |
| RHG06_HUMAN | Rho GTPase-activating protein 6 | 0 | 1 | −1.88 |
| | OS = Homo sapiens GN = ARHGAP6 | | | |
| | PE = 1 SV = 3 | | | |
| RL17_HUMAN | 60S ribosomal protein L17 | 0.28 | 1 | −1.12 |
| | OS = Homo sapiens GN = RPL17 | | | |
| | PE = 1 SV = 3 | | | |
| RL18_HUMAN | 60S ribosomal protein L18 | 0.99 | 2 | 1.00 |
| | OS = Homo sapiens GN = RPL18 | | | |
| | PE = 1 SV = 2 | | | |
| RL22_HUMAN | 60S ribosomal protein L22 | 0.98 | 2 | −1.08 |
| | OS = Homo sapiens GN = RPL22 | | | |
| | PE = 1 SV = 2 | | | |
| RL23_HUMAN | 60S ribosomal protein L23 | 1 | 7 | −1.07 |
| | OS = Homo sapiens GN = RPL23 | | | |
| | PE = 1 SV = 1 | | | |
| RL23A_HUMAN | 60S ribosomal protein L23a | 0.83 | 1 | −1.01 |
| | OS = Homo sapiens GN = RPL23A | | | |
| | PE = 1 SV = 1 | | | |
| RL24_HUMAN | 60S ribosomal protein L24 | 1 | 2 | −1.16 |
| | OS = Homo sapiens GN = RPL24 | | | |
| | PE = 1 SV = 1 | | | |
| RL27A_HUMAN | 60S ribosomal protein L27a | 1 | 2 | −1.20 |
| | OS = Homo sapiens GN = RPL27A | | | |
| | PE = 1 SV = 2 | | | |
| RL31_HUMAN | 60S ribosomal protein L31 | 0.99 | 3 | −1.08 |
| | OS = Homo sapiens GN = RPL31 | | | |
| | PE = 1 SV = 1 | | | |
| RL35_HUMAN | 60S ribosomal protein L35 | 0.34 | 1 | −1.23 |
| | OS = Homo sapiens GN = RPL35 | | | |
| | PE = 1 SV = 2 | | | |
| RL6_HUMAN | 60S ribosomal protein L6 | 1 | 4 | −1.18 |
| | OS = Homo sapiens GN = RPL6 | | | |
| | PE = 1 SV = 3 | | | |
| RL7_HUMAN | 60S ribosomal protein L7 | 0.74 | 1 | −1.07 |
| | OS = Homo sapiens GN = RPL7 | | | |
| | PE = 1 SV = 1 | | | |
| RLA2_HUMAN | 60S acidic ribosomal protein P2 | 1 | 7 | −1.16 |
| | OS = Homo sapiens GN = RPLP2 | | | |
| | PE = 1 SV = 1 | | | |
| ROA1_HUMAN | Heterogeneous nuclear | 0.83 | 1 | 1.16 |
| | ribonucleoprotein A1 OS = | | | |
| | Homo sapiens GN = HNRNPA1 PE = 1 | | | |
| | SV = 4 | | | |
| ROA2_HUMAN | Heterogeneous nuclear | 1 | 4 | 1.11 |
| | ribonucleoproteins A2/B1 | | | |
| | OS = Homo sapiens | | | |
| | GN = HNRNPA2B1 PE = 1 SV = 2 | | | |
| ROA3_HUMAN | Heterogeneous nuclear | 0 | 1 | 1.09 |
| | ribonucleoprotein A3 OS = | | | |
| | Homo sapiens GN = HNRNPA3 PE = 1 | | | |
| | SV = 2 | | | |
| RPE_HUMAN | Ribulose-phosphate 3-epimerase | 0.89 | 1 | 1.06 |
| | OS = Homo sapiens GN = RPE PE = 1 | | | |
| | SV = 1 | | | |
| RRBP1_HUMAN | Ribosome-binding protein 1 | 0.82 | 1 | 1.29 |
| | OS = Homo sapiens GN = RRBP1 | | | |
| | PE = 1 SV = 4 | | | |
| RS13_HUMAN | 40S ribosomal protein S13 | 0.83 | 1 | 1.04 |
| | OS = Homo sapiens GN = RPS13 | | | |
| | PE = 1 SV = 2 | | | |
| RS15_HUMAN | 40S ribosomal protein S15 | 1 | 2 | 1.03 |
| | OS = Homo sapiens GN = RPS15 | | | |
| | PE = 1 SV = 2 | | | |
| RS18_HUMAN | 40S ribosomal protein S18 | 0.99 | 3 | −1.04 |
| | OS = Homo sapiens GN = RPS18 | | | |
| | PE = 1 SV = 3 | | | |
| RS24_HUMAN | 40S ribosomal protein S24 | 0.69 | 1 | −1.16 |
| | OS = Homo sapiens GN = RPS24 | | | |
| | PE = 1 SV = 1 | | | |
| RS25_HUMAN | 40S ribosomal protein S25 | 0.96 | 1 | −1.22 |
| | OS = Homo sapiens GN = RPS25 | | | |
| | PE = 1 SV = 1 | | | |
| RS6_HUMAN | 40S ribosomal protein S6 | 1 | 4 | −1.09 |
| | OS = Homo sapiens GN = RPS6 | | | |
| | PE = 1 SV = 1 | | | |
| RS8_HUMAN | 40S ribosomal protein S8 | 1 | 3 | −1.23 |
| | OS = Homo sapiens GN = RPS8 | | | |
| | PE = 1 SV = 2 | | | |
| RT36_HUMAN | 28S ribosomal protein S36, | 1 | 9 | −1.11 |
| | mitochondrial OS = Homo sapiens | | | |
| | GN = MRPS36 PE = 1 SV = 2 | | | |
| S10A1_HUMAN | Protein S100-A1 OS = | 1 | 3 | 1.11 |
| | Homo sapiens GN = S100A1 PE = 1 | | | |
| | SV = 2 | | | |
| SAA_HUMAN | Serum amyloid A protein | 0.99 | 2 | 1.14 |
| | OS = Homo sapiens GN = SAA1 | | | |
| | PE = 1 SV = 2 | | | |
| SAMP_HUMAN | Serum amyloid P-component | 1 | 10 | 1.09 |
| | OS = Homo sapiens GN = APCS | | | |
| | PE = 1 SV = 2 | | | |
| SAP_HUMAN | Proactivator polypeptide | 1 | 5 | −1.07 |
| | OS = Homo sapiens GN = PSAP | | | |
| | PE = 1 SV = 2 | | | |
| SDPR_HUMAN | Serum deprivation-response | 1 | 14 | −1.08 |
| | protein OS = Homo sapiens | | | |
| | GN = SDPR PE = 1 SV = 3 | | | |
| SEPT7_HUMAN | Septin-7 OS = Homo sapiens | 0.93 | 1 | 1.26 |
| | GN = SEPT7 PE = 1 SV = 2 | | | |
| SET_HUMAN | Protein SET OS = Homo sapiens | 0.7 | 1 | −1.00 |
| | GN = SET PE = 1 SV = 3 | | | |
| SGCB_HUMAN | Beta-sarcoglycan OS = | 1 | 8 | −1.17 |
| | Homo sapiens GN = SGCB PE = 1 | | | |
| | SV = 1 | | | |
| SGCG_HUMAN | Gamma-sarcoglycan OS = | 0.99 | 2 | −1.03 |
| | Homo sapiens GN = SGCG PE = 1 | | | |
| | SV = 3 | | | |
| SH3BG_HUMAN | SH3 domain-binding glutamic | 1 | 7 | −1.12 |
| | acid-rich protein OS = | | | |
| | Homo sapiens GN = SH3BGR PE = 1 | | | |
| | SV = 3 | | | |
| SIAE_HUMAN | Sialate O-acetylesterase | 1 | 10 | −1.21 |
| | OS = Homo sapiens GN = SIAE | | | |
| | PE = 2 SV = 1 | | | |
| SODC_HUMAN | Superoxide dismutase [Cu—Zn] | 1 | 19 | −1.12 |
| | OS = Homo sapiens GN = SOD1 | | | |
| | PE = 1 SV = 2 | | | |
| SODE_HUMAN | Extracellular superoxide | 1 | 12 | −1.04 |
| | dismutase [Cu—Zn] OS = | | | |
| | Homo sapiens GN = SOD3 PE = 1 | | | |
| | SV = 2 | | | |
| SODM_HUMAN | Superoxide dismutase [Mn], | 1 | 15 | 1.11 |
| | mitochondrial OS = Homo sapiens | | | |
| | GN = SOD2 PE = 1 SV = 2 | | | |
| SRBS2_HUMAN | Sorbin and SH3 domain- | 0.96 | 1 | 1.44 |
| | containing protein 2 OS = | | | |
| | Homo sapiens GN = SORBS2 PE = 1 | | | |
| | SV = 3 | | | |
| SRCA_HUMAN | Sarcalumenin OS = Homo sapiens | 1 | 23 | −1.12 |
| | GN = SRL PE = 2 SV = 2 | | | |
| SRCH_HUMAN | Sarcoplasmic reticulum histidine- | 1 | 23 | −1.36 |
| | rich calcium-binding protein | | | |
| | OS = Homo sapiens GN = HRC PE = 2 | | | |
| | SV = 1 | | | |
| STIM2_HUMAN | Stromal interaction molecule 2 | 0.3 | 1 | 14.56 |
| | OS = Homo sapiens GN = STIM2 | | | |
| | PE = 1 SV = 2 | | | |
| SUCA_HUMAN | Succinyl-CoA ligase [GDP- | 1 | 9 | −1.01 |
| | forming] subunit alpha, | | | |
| | mitochondrial OS = Homo sapiens | | | |
| | GN = SUCLG1 PE = 1 SV = 4 | | | |
| TAGL_HUMAN | Transgelin OS = Homo sapiens | 0.52 | 1 | 3.24 |
| | GN = TAGLN PE = 1 SV = 4 | | | |
| TBA1B_HUMAN | Tubulin alpha-1B chain OS = | 1 | 3 | 1.19 |
| | Homo sapiens GN = TUBA1B PE = 1 | | | |
| | SV = 1 | | | |
| TBB2A_HUMAN | Tubulin beta-2A chain OS = | 0.98 | 2 | 1.11 |
| | Homo sapiens GN = TUBB2A PE = 1 | | | |
| | SV = 1 | | | |
| TBB2C_HUMAN | Tubulin beta-2C chain OS = | 0.98 | 3 | 1.13 |
| | Homo sapiens GN = TUBB2C PE = 1 | | | |
| | SV = 1 | | | |
| TBB3_HUMAN | Tubulin beta-3 chain OS = | 0.86 | 1 | −1.06 |
| | Homo sapiens GN = TUBB3 PE = 1 | | | |
| | SV = 2 | | | |
| TBB4_HUMAN | Tubulin beta-4 chain OS = | 0.93 | 1 | −1.17 |
| | Homo sapiens GN = TUBB4 PE = 1 | | | |
| | SV = 2 | | | |
| TBB5_HUMAN | Tubulin beta chain OS = | 0.99 | 19 | 1.10 |
| | Homo sapiens GN = TUBB PE = 1 | | | |
| | SV = 2 | | | |
| TBB6_HUMAN | Tubulin beta-6 chain OS = | 0.89 | 2 | 1.05 |
| | Homo sapiens GN = TUBB6 PE = 1 | | | |
| | SV = 1 | | | |
| TBB8_HUMAN | Tubulin beta-8 chain OS = | 0.98 | 2 | 1.09 |
| | Homo sapiens GN = TUBB8 PE = 1 | | | |
| | SV = 2 | | | |
| TBB8B_HUMAN | Tubulin beta-8 chain B OS = | 0.65 | 1 | −1.27 |
| | Homo sapiens PE = 1 SV = 1 | | | |
| TCTP_HUMAN | Translationally-controlled tumor | 0.63 | 1 | −1.07 |
| | protein OS = Homo sapiens | | | |
| | GN = TPT1 PE = 1 SV = 1 | | | |
| TEBP_HUMAN | Prostaglandin E synthase 3 | 1 | 4 | −1.16 |
| | OS = Homo sapiens GN = PTGES3 | | | |
| | PE = 1 SV = 1 | | | |
| TELT_HUMAN | Telethonin OS = Homo sapiens | 1 | 4 | 1.11 |
| | GN = TCAP PE = 1 SV = 1 | | | |
| TFAM_HUMAN | Transcription factor A, | 0.99 | 2 | −1.09 |
| | mitochondrial OS = Homo sapiens | | | |
| | GN = TFAM PE = 1 SV = 1 | | | |
| TGM7_HUMAN | Protein-glutamine gamma- | 0.83 | 1 | 1.01 |
| | glutamyltransferase Z OS = | | | |
| | Homo sapiens GN = TGM7 PE = 2 | | | |
| | SV = 1 | | | |
| THIL_HUMAN | Acetyl-CoA acetyltransferase, | 1 | 2 | 1.25 |
| | mitochondrial OS = Homo sapiens | | | |
| | GN = ACAT1 PE = 1 SV = 1 | | | |
| THIM_HUMAN | 3-ketoacyl-CoA thiolase, | 1 | 10 | 1.13 |
| | mitochondrial OS = Homo sapiens | | | |
| | GN = ACAA2 PE = 1 SV = 2 | | | |
| THIO_HUMAN | Thioredoxin OS = Homo sapiens | 0 | 1 | −1.19 |
| | GN = TXN PE = 1 SV = 3 | | | |
| THRB_HUMAN | Prothrombin OS = Homo sapiens | 1 | 6 | −1.33 |
| | GN = F2 PE = 1 SV = 2 | | | |
| TI21L_HUMAN | TIM21-like protein, mitochondrial | 0.59 | 1 | −1.30 |
| | OS = Homo sapiens GN = C18orf55 | | | |
| | PE = 2 SV = 1 | | | |
| TINAL_HUMAN | Tubulointerstitial nephritis | 1 | 15 | 1.05 |
| | antigen-like OS = Homo sapiens | | | |
| | GN = TINAGL1 PE = 1 SV = 1 | | | |
| TLE3_HUMAN | Transducin-like enhancer protein | 0.33 | 1 | −2.21 |
| | 3 OS = Homo sapiens GN = TLE3 | | | |
| | PE = 1 SV = 2 | | | |
| TM40L_HUMAN | Mitochondrial import receptor | 0 | 1 | −1.84 |
| | subunit TOM40B OS = | | | |
| | Homo sapiens GN = TOMM40L PE = 2 | | | |
| | SV = 1 | | | |
| TMEDA_HUMAN | Transmembrane emp24 domain- | 0.81 | 1 | −1.02 |
| | containing protein 10 OS = | | | |
| | Homo sapiens GN = TMED10 PE = 1 | | | |
| | SV = 2 | | | |
| TNNC1_HUMAN | Troponin C, slow skeletal and | 1 | 42 | −1.12 |
| | cardiac muscles OS = | | | |
| | Homo sapiens GN = TNNC1 PE = 1 | | | |
| | SV = 1 | | | |
| TNNI3_HUMAN | Troponin I, cardiac muscle | 1 | 41 | −1.05 |
| | OS = Homo sapiens GN = TNNI3 | | | |
| | PE = 1 SV = 3 | | | |
| TNNT1_HUMAN | Troponin T, slow skeletal muscle | 1 | 5 | −1.26 |
| | OS = Homo sapiens GN = TNNT1 | | | |
| | PE = 1 SV = 4 | | | |
| TNNT2_HUMAN | Troponin T, cardiac muscle | 1 | 66 | −1.16 |
| | OS = Homo sapiens GN = TNNT2 | | | |
| | PE = 1 SV = 3 | | | |
| TPIS_HUMAN | Triosephosphate isomerase | 1 | 23 | 1.11 |
| | OS = Homo sapiens GN = TPI1 | | | |
| | PE = 1 SV = 2 | | | |
| TPM1_HUMAN | Tropomyosin alpha-1 chain | 1 | 73 | −1.09 |
| | OS = Homo sapiens GN = TPM1 | | | |
| | PE = 1 SV = 2 | | | |
| TPM2_HUMAN | Tropomyosin beta chain | 1 | 62 | −1.10 |
| | OS = Homo sapiens GN = TPM2 | | | |
| | PE = 1 SV = 1 | | | |
| TPM3_HUMAN | Tropomyosin alpha-3 chain | 1 | 10 | −1.15 |
| | OS = Homo sapiens GN = TPM3 | | | |
| | PE = 1 SV = 1 | | | |
| TPM3L_HUMAN | Putative tropomyosin alpha-3 | 0.39 | 1 | 2.18 |
| | chain-like protein OS = | | | |
| | Homo sapiens PE = 5 SV = 2 | | | |
| TPM4_HUMAN | Tropomyosin alpha-4 chain | 1 | 4 | 1.08 |
| | OS = Homo sapiens GN = TPM4 | | | |
| | PE = 1 SV = 3 | | | |
| TPP1_HUMAN | Tripeptidyl-peptidase 1 OS = | 1 | 7 | −1.43 |
| | Homo sapiens GN = TPP1 PE = 1 | | | |
| | SV = 2 | | | |
| TPPP_HUMAN | Tubulin polymerization- | 0.83 | 1 | −1.04 |
| | promoting protein OS = | | | |
| | Homo sapiens GN = TPPP PE = 1 | | | |
| | SV = 1 | | | |
| TRFE_HUMAN | Serotransferrin OS = Homo sapiens | 1 | 108 | −1.09 |
| | GN = TF PE = 1 SV = 2 | | | |
| TTHY_HUMAN | Transthyretin OS = Homo sapiens | 1 | 7 | 1.02 |
| | GN = TTR PE = 1 SV = 1 | | | |
| UB2L3_HUMAN | Ubiquitin-conjugating enzyme E2 | 1 | 3 | −1.04 |
| | L3 OS = Homo sapiens GN = UBE2L3 | | | |
| | PE = 1 SV = 1 | | | |
| UBIQ_HUMAN | Ubiquitin OS = Homo sapiens | 1 | 6 | 1.15 |
| | GN = RPS27A PE = 1 SV = 1 | | | |
| UCRI_HUMAN | Cytochrome b-c1 complex | 1 | 11 | −1.05 |
| | subunit Rieske, mitochondrial | | | |
| | OS = Homo sapiens GN = UQCRFS1 | | | |
| | PE = 1 SV = 2 | | | |
| VDAC1_HUMAN | Voltage-dependent anion- | 1 | 10 | 1.01 |
| | selective channel protein 1 | | | |
| | OS = Homo sapiens GN = VDAC1 | | | |
| | PE = 1 SV = 2 | | | |
| VDAC2_HUMAN | Voltage-dependent anion- | 1 | 14 | −1.03 |
| | selective channel protein 2 | | | |
| | OS = Homo sapiens GN = VDAC2 | | | |
| | PE = 1 SV = 2 | | | |
| VDAC3_HUMAN | Voltage-dependent anion- | 1 | 16 | −1.04 |
| | selective channel protein 3 | | | |
| | OS = Homo sapiens GN = VDAC3 | | | |
| | PE = 1 SV = 1 | | | |
| VIME_HUMAN | Vimentin OS = Homo sapiens | 1 | 18 | 1.33 |
| | GN = VIM PE = 1 SV = 4 | | | |
| VTDB_HUMAN | Vitamin D-binding protein | 1 | 15 | −1.07 |
| | OS = Homo sapiens GN = GC PE = 1 | | | |
| | SV = 1 | | | |
| VTNC_HUMAN | Vitronectin OS = Homo sapiens | 1 | 8 | −1.06 |
| | GN = VTN PE = 1 SV = 1 | | | |
| WDR46_HUMAN | WD repeat-containing protein 46 | 0 | 1 | 1.21 |
| | OS = Homo sapiens GN = WDR46 | | | |
| | PE = 1 SV = 2 | | | |
| YD007_HUMAN | Coiled-coil domain-containing | 0.43 | 1 | 1.05 |
| | protein FLJ25770 OS = | | | |
| | Homo sapiens PE = 2 SV = 2 | | | |
| ZA2G_HUMAN | Zinc-alpha-2-glycoprotein | 1 | 9 | −1.29 |
| | OS = Homo sapiens GN = AZGP1 | | | |
| | PE = 1 SV = 1 | | | |
| ZN350_HUMAN | Zinc finger protein 350 OS = | 0.81 | 1 | −1.12 |
| | Homo sapiens GN = ZNF350 PE = 1 | | | |
| | SV = 2 | | | |

| | NIF v IF | IF v NF | IF v NF | NIF v NF | NIF v NF |
|---|---|---|---|---|---|
| Primary Protein | p-value | Fold | p-value | Fold | p-value |
| Name | (ANOVA) | Change | (ANOVA) | Change | (ANOVA) |
| 1433B_HUMAN | 0.9639 | 1.06 | 0.6925 | 1.08 | 0.3942 |
| 1433E_HUMAN | 0.9715 | −1.08 | 0.8024 | −1.06 | 0.806 |
| 1433F_HUMAN | 0.6795 | −1.24 | 0.4899 | −1.53 | 0.2222 |
| 1433G_HUMAN | 0.7845 | −1.18 | 0.4175 | −1.03 | 0.8433 |
| 1433T_HUMAN | 0.8696 | 1.16 | 0.5754 | 1.05 | 0.8606 |
| 1433Z_HUMAN | 0.9208 | −1.13 | 0.6166 | −1.05 | 0.6657 |
| A1AG1_HUMAN | 0.8797 | 1.43 | 0.3017 | 1.25 | 0.5405 |
| A1AG2_HUMAN | 0.773 | 1.53 | 0.0973 | 1.31 | 0.2933 |
| A1AT_HUMAN | 0.9715 | 1.29 | 0.199 | 1.25 | 0.4372 |
| A1BG_HUMAN | 0.7451 | 1.77 | 0.0381 | 1.48 | 0.1718 |
| A26CA_HUMAN | 0.9715 | 1.01 | 0.8758 | 1.00 | 0.8702 |
| A26CB_HUMAN | 0.8696 | −1.08 | 0.5193 | 1.00 | 0.9742 |
| A2GL_HUMAN | 0.7135 | 1.02 | 0.972 | −1.43 | 0.3986 |
| A2MG_HUMAN | 0.6795 | 2.31 | 0.00010051 | 1.84 | 0.00020478 |
| AACT_HUMAN | 0.9355 | −1.07 | 0.7869 | −1.19 | 0.6122 |
| AATC_HUMAN | 0.8696 | −1.19 | 0.4699 | −1.37 | 0.1116 |
| ABCAD_HUMAN | 0.9715 | 1.12 | 0.4097 | 1.11 | 0.4198 |
| ACADM_HUMAN | 0.2107 | −1.13 | 0.5857 | 1.21 | 0.4453 |
| ACPM_HUMAN | 0.9715 | −1.28 | 0.1832 | −1.33 | 0.2511 |
| ACTA_HUMAN | 0.9871 | −1.39 | 0.3341 | −1.40 | 0.1351 |
| ACTB_HUMAN | 0.6795 | −1.11 | 0.6569 | 1.15 | 0.5046 |
| ACTBL_HUMAN | 0.8696 | −1.15 | 0.0572 | −1.11 | 0.1445 |
| ACTK_HUMAN | 0.9404 | −1.46 | 0.0234 | −1.40 | 0.00018155 |
| ACTS_HUMAN | 0.9355 | −1.18 | 0.4627 | −1.14 | 0.5046 |
| ADH1_YEAST | 0.8057 | 1.07 | 0.2678 | 1.11 | 0.0217 |
| ADH1G_HUMAN | 0.9715 | 1.29 | 0.7256 | 1.23 | 0.7395 |
| ADIP_HUMAN | 0.3444 | −1.41 | 0.0556 | −1.90 | 8.5821E−07 |
| ADIPO_HUMAN | 0.9984 | −1.23 | 0.4097 | −1.24 | 0.3126 |
| AHNK2_HUMAN | 0.8696 | 1.08 | 0.8625 | −1.09 | 0.7262 |
| AIFM1_HUMAN | 0.9355 | −1.47 | 0.4097 | −1.36 | 0.409 |
| AIM1_HUMAN | 0.7147 | 1.56 | 0.141 | 1.14 | 0.6682 |
| ALBU_HUMAN | 0.6795 | 1.36 | 5.0222E−07 | 1.46 | 1.8704E−06 |
| ALDOA_HUMAN | 0.8171 | −1.25 | 0.4097 | −1.13 | 0.5405 |
| ALDOC_HUMAN | 0.9715 | −1.08 | 0.7145 | −1.09 | 0.5434 |
| ALPK2_HUMAN | 0.7269 | −1.12 | 0.7056 | 1.14 | 0.6445 |
| AMBP_HUMAN | 0.4349 | 1.50 | 0.0541 | 1.05 | 0.8266 |
| ANGT_HUMAN | 0.8696 | 1.29 | 0.6441 | 1.56 | 0.3508 |
| ANT3_HUMAN | 0.4301 | 1.27 | 0.4259 | −1.04 | 0.794 |
| ANXA2_HUMAN | 0.945 | 1.13 | 0.6583 | 1.13 | 0.5434 |
| ANXA5_HUMAN | 0.9432 | 1.28 | 0.4175 | 1.45 | 0.0108 |
| AOC3_HUMAN | 0.9639 | 1.13 | 0.7823 | 1.11 | 0.7395 |
| APOA4_HUMAN | 0.8879 | 1.27 | 0.3137 | 1.19 | 0.2523 |
| APOH_HUMAN | 0.783 | 1.56 | 0.0814 | 1.29 | 0.4636 |
| APOOL_HUMAN | 0.9338 | −1.23 | 0.21 | −1.17 | 0.2398 |
| ARGFX_HUMAN | 0.6795 | 1.76 | 0.0227 | 1.27 | 0.3458 |
| ARHGH_HUMAN | 0.8195 | −1.17 | 0.4914 | −1.37 | 0.1162 |
| ARHGJ_HUMAN | 0.6795 | −1.05 | 0.7878 | 1.08 | 0.5884 |
| ASAH1_HUMAN | 0.0228 | −1.19 | 0.1322 | −1.53 | 8.2018E−08 |
| ASPN_HUMAN | 0.6795 | 1.84 | 0.2098 | 3.03 | 0.0154 |
| AT1B1_HUMAN | 0.6795 | −1.17 | 0.1302 | −1.38 | 0.0229 |
| ATPB_HUMAN | 0.9639 | −1.26 | 0.6766 | −1.23 | 0.2521 |
| ATPD_HUMAN | 0.726 | −1.66 | 0.1461 | −2.22 | 0.00061632 |
| ATPG_HUMAN | 0.4996 | −1.47 | 0.0028 | −1.29 | 0.014 |
| ATPK_HUMAN | 0.8791 | −1.50 | 0.013 | −1.66 | 0.0283 |
| ATS20_HUMAN | 0.9319 | 1.47 | 0.000050712 | 1.54 | 0.0049 |
| AXA2L_HUMAN | 0.9404 | −1.02 | 0.9816 | 1.03 | 0.7356 |
| BASI_HUMAN | 0.8077 | −1.19 | 0.274 | −1.35 | 0.0495 |
| BIEA_HUMAN | 0.8879 | 1.50 | 0.0028 | 1.44 | 0.0018 |
| BST2_HUMAN | 0.8791 | −1.35 | 0.4508 | −1.59 | 0.178 |
| BUD13_HUMAN | 0.9432 | 1.56 | 0.00077578 | 1.60 | 0.000090322 |
| C1QBP_HUMAN | 0.6795 | −1.17 | 0.0221 | −1.33 | 0.0045 |
| CAD13_HUMAN | 0.8879 | −1.25 | 0.3266 | −1.39 | 0.1439 |
| CADH2_HUMAN | 0.7451 | −1.08 | 0.732 | −1.24 | 0.1633 |
| CAH1_HUMAN | 0.6511 | 7.24 | 0.0016 | 4.06 | 0.00031396 |
| CAH3_HUMAN | 0.3398 | 2.48 | 0.3041 | 7.02 | 0.0119 |
| CALD1_HUMAN | 0.6795 | 1.03 | 0.8858 | 1.21 | 0.1439 |
| CALM_HUMAN | 0.9525 | −1.15 | 0.21 | −1.12 | 0.1433 |
| CALR_HUMAN | 0.6795 | −1.11 | 0.3137 | −1.20 | 0.00069128 |
| CALU_HUMAN | 0.6795 | −1.48 | 0.036 | −1.75 | 4.996E−14 |
| CAPZB_HUMAN | 0.9715 | −1.07 | 0.8399 | −1.05 | 0.8249 |
| CASQ2_HUMAN | 0.3175 | −1.03 | 0.84 | −1.27 | 0.0289 |
| CATB_HUMAN | 0.8879 | −1.24 | 0.0941 | −1.17 | 0.2352 |
| CATD_HUMAN | 0.9404 | −1.33 | 0.0234 | −1.37 | 0.0065 |
| CAZA2_HUMAN | 0.8696 | −2.00 | 0.0031 | −1.80 | 0.0051 |
| CCD57_HUMAN | 0.9117 | 2.05 | 0.1331 | 1.65 | 0.4426 |
| CD36_HUMAN | 0.0842 | −1.01 | 0.6799 | −1.45 | 0.0827 |
| CD59_HUMAN | 0.8696 | −1.22 | 0.4263 | −1.33 | 0.0304 |
| CD99_HUMAN | 0.7451 | −1.24 | 0.2813 | −1.53 | 0.1155 |
| CERU_HUMAN | 0.8214 | 1.83 | 0.0063 | 2.09 | 0.004 |
| CFAB_HUMAN | 0.9438 | 1.79 | 0.1415 | 1.70 | 0.1606 |
| CFAH_HUMAN | 0.9355 | 1.61 | 0.0528 | 1.46 | 0.1439 |
| CH10_HUMAN | 0.6795 | −1.19 | 0.0013 | −1.12 | 0.1155 |
| CHCH3_HUMAN | 0.7519 | −1.16 | 0.0516 | −1.28 | 0.0258 |
| CHDH_HUMAN | 0.7845 | −1.21 | 0.4561 | −1.47 | 0.141 |
| CHST7_HUMAN | 0.6795 | −1.42 | 0.6166 | −2.21 | 0.1015 |
| CISD1_HUMAN | 0.6795 | −1.29 | 0.01 | −1.49 | 0.0024 |
| CK067_HUMAN | 0.7451 | −1.21 | 0.6166 | −1.60 | 0.2352 |
| CLCB_HUMAN | 0.6795 | 1.05 | 0.6925 | −1.07 | 0.53 |
| CLUS_HUMAN | 0.6795 | 1.47 | 0.1197 | 1.19 | 0.53 |
| CMA1_HUMAN | 0.6795 | −1.30 | 0.4717 | 1.05 | 0.8186 |
| CN045_HUMAN | 0.9871 | −1.08 | 0.6939 | −1.07 | 0.6677 |
| CNBP_HUMAN | 0.783 | −1.37 | 0.1302 | −1.62 | 0.0113 |
| CO4A1_HUMAN | 0.9639 | 1.20 | 0.6738 | 1.19 | 0.5434 |
| CO4A2_HUMAN | 0.6795 | −1.28 | 0.1415 | −1.04 | 0.8963 |
| CO6A1_HUMAN | 0.6795 | 1.29 | 0.0187 | 1.46 | 0.00034768 |
| CO6A2_HUMAN | 0.7398 | 1.38 | 0.2579 | 1.85 | 0.0235 |
| CO6A3_HUMAN | 0.6795 | 1.75 | 0.1832 | 2.93 | 0.0056 |
| CO9_HUMAN | 0.8791 | 1.56 | 0.0775 | 1.35 | 0.3986 |
| COF2_HUMAN | 0.3444 | −1.35 | 0.4175 | 1.17 | 0.5263 |
| COFA1_HUMAN | 0.6795 | 1.02 | 0.9585 | −1.21 | 0.3462 |
| COG1_HUMAN | 0.472 | −1.39 | 0.0023 | −1.11 | 0.5405 |
| COIA1_HUMAN | 0.9639 | 1.27 | 0.4997 | 1.27 | 0.4049 |
| CORL2_HUMAN | 0.8696 | −1.17 | 0.737 | 1.04 | 0.8963 |
| COX2_HUMAN | 0.8696 | −1.48 | 0.4175 | −1.24 | 0.4547 |
| COX41_HUMAN | 0.8879 | −1.42 | 0.0038 | −1.33 | 0.1118 |
| COX5A_HUMAN | 0.8608 | −1.53 | 0.0018 | −1.40 | 0.0838 |
| COX5B_HUMAN | 0.8696 | −1.41 | 0.0124 | −1.31 | 0.2533 |
| CRIP2_HUMAN | 0.9715 | 1.06 | 0.7313 | 1.05 | 0.5936 |
| CRYAB_HUMAN | 0.7497 | −1.02 | 0.8791 | −1.12 | 0.2523 |
| CSPG2_HUMAN | 0.8671 | 1.58 | 0.4856 | 2.37 | 0.1351 |
| CSRP1_HUMAN | 0.7451 | −1.09 | 0.8209 | 1.18 | 0.6962 |
| CSRP3_HUMAN | 0.7135 | −1.00 | 0.9957 | −1.12 | 0.5434 |
| CX6B1_HUMAN | 0.8671 | −1.37 | 0.0211 | −1.26 | 0.2996 |
| CX7A1_HUMAN | 0.9715 | −1.32 | 0.1694 | −1.33 | 0.3426 |
| CY1_HUMAN | 0.8879 | −1.23 | 0.5949 | −1.34 | 0.1441 |
| CYC_HUMAN | 0.945 | −1.17 | 0.1627 | −1.20 | 0.2564 |
| DAG1_HUMAN | 0.7451 | −1.15 | 0.4097 | −1.37 | 0.0859 |
| DECR_HUMAN | 0.9154 | 1.20 | 0.7517 | 1.40 | 0.3286 |
| DERM_HUMAN | 0.6795 | 1.40 | 0.3787 | 2.08 | 0.0283 |
| DESM_HUMAN | 0.8696 | 1.07 | 0.8316 | 1.23 | 0.3995 |
| DHSB_HUMAN | 0.9715 | −1.22 | 0.2595 | −1.24 | 0.1357 |
| DLDH_HUMAN | 0.8696 | −1.15 | 0.646 | −1.04 | 0.849 |
| DMC1_HUMAN | 0.8696 | 1.25 | 0.4508 | 1.06 | 0.8607 |
| DSG2_HUMAN | 0.9355 | −1.27 | 0.4892 | −1.18 | 0.53 |
| DYH11_HUMAN | 0.8879 | 1.12 | 0.6925 | −1.09 | 0.8199 |
| DYXC1_HUMAN | 0.5535 | 1.03 | 0.4899 | −1.12 | 0.2523 |
| ECH1_HUMAN | 0.7321 | −1.33 | 0.4376 | −1.07 | 0.6692 |
| ECHM_HUMAN | 0.9438 | −1.12 | 0.7456 | −1.03 | 0.7888 |
| ECT2_HUMAN | 0.9715 | −1.43 | 0.0192 | −1.41 | 0.0118 |
| EF1A2_HUMAN | 0.945 | −1.27 | 0.4175 | −1.34 | 0.2475 |
| EF1A3_HUMAN | 0.9355 | 1.17 | 0.5943 | 1.23 | 0.3986 |
| EF1B_HUMAN | 0.6795 | 1.10 | 0.4508 | −1.03 | 0.8607 |
| EFTU_HUMAN | 0.8696 | −1.28 | 0.0528 | −1.21 | 0.0329 |
| ENPL_HUMAN | 0.8879 | −1.05 | 0.7823 | 1.01 | 0.9549 |
| EPDR1_HUMAN | 0.1803 | −1.25 | 0.4899 | −2.15 | 0.0094 |
| ES1_HUMAN | 0.8671 | −1.27 | 0.1603 | −1.38 | 0.0258 |
| ETFA_HUMAN | 0.9715 | −1.22 | 0.4376 | −1.23 | 0.2474 |
| F168B_HUMAN | 0.9744 | 1.96 | 0.0133 | 1.94 | 0.0118 |
| FA9_HUMAN | 0.3444 | −1.34 | 0.4376 | −2.05 | 0.00061632 |
| FABP4_HUMAN | 0.9639 | −1.02 | 0.9585 | 1.02 | 0.9413 |
| FABP5_HUMAN | 0.8696 | −1.22 | 0.3298 | −1.11 | 0.6657 |
| FABPH_HUMAN | 0.9715 | −1.22 | 0.0225 | −1.24 | 0.1155 |
| FB5L3_HUMAN | 0.8753 | −1.01 | 0.9816 | −1.10 | 0.6657 |
| FBLN1_HUMAN | 0.8879 | 1.90 | 0.0279 | 2.16 | 0.0468 |
| FBLN2_HUMAN | 0.5797 | 1.48 | 0.199 | 2.52 | 0.0071 |
| FBLN3_HUMAN | 0.5784 | 1.31 | 0.3094 | 2.25 | 0.0172 |
| FBN1_HUMAN | 0.03 | −1.26 | 0.1957 | 1.42 | 0.0133 |
| FETUA_HUMAN | 0.9404 | 1.66 | 1.0691E−11 | 1.71 | 1.3418E−06 |
| FHL1_HUMAN | 0.783 | −1.06 | 0.84 | 1.05 | 0.8646 |
| FHL2_HUMAN | 0.9871 | −1.19 | 0.4899 | −1.22 | 0.4453 |
| FIBA_HUMAN | 0.8555 | 1.56 | 0.1553 | 1.81 | 0.0708 |
| FIBB_HUMAN | 0.8057 | 1.65 | 0.0869 | 1.96 | 0.0217 |
| FIBG_HUMAN | 0.783 | 1.89 | 0.0528 | 2.29 | 0.0142 |
| FKBP3_HUMAN | 0.5029 | −1.25 | 0.0381 | −1.57 | 0.00028676 |
| FMOD_HUMAN | 0.6795 | 1.50 | 0.575 | 2.98 | 0.1005 |
| FRAS1_HUMAN | 0.9438 | −1.22 | 0.4945 | −1.33 | 0.4597 |
| FRIH_HUMAN | 0.1996 | −1.78 | 0.0067 | −1.18 | 0.4597 |
| FSTL4_HUMAN | 0.8057 | −1.56 | 0.21 | −1.26 | 0.5534 |
| FUMH_HUMAN | 0.8696 | −1.31 | 0.6173 | −1.14 | 0.8702 |
| G3P_HUMAN | 0.9832 | −1.06 | 0.732 | −1.08 | 0.6122 |
| GCSH_HUMAN | 0.5039 | −1.27 | 0.0927 | −1.73 | 0.0074 |
| GDIR1_HUMAN | 0.7445 | −1.09 | 0.4316 | −1.16 | 0.0464 |
| GDIR2_HUMAN | 0.9715 | 1.15 | 0.4899 | 1.13 | 0.6187 |
| GGT2_HUMAN | 0.8431 | −1.06 | 0.84 | −1.18 | 0.0493 |
| GLYG_HUMAN | 0.472 | −1.03 | 0.9014 | −1.32 | 0.028 |
| GPC1_HUMAN | 0.6795 | −1.04 | 0.921 | −1.42 | 0.2212 |
| GPNMB_HUMAN | 0.6795 | −1.24 | 0.4097 | −1.46 | 0.0213 |
| GRB1L_HUMAN | 0.6795 | −1.38 | 0.2727 | −1.71 | 0.0013 |
| GRP75_HUMAN | 0.6795 | −1.31 | 0.4258 | −1.04 | 0.8126 |
| GRP78_HUMAN | 0.8879 | −1.13 | 0.6588 | −1.03 | 0.9375 |
| GSTO1_HUMAN | 0.9129 | 1.19 | 0.7593 | 1.38 | 0.4174 |
| H10_HUMAN | 0.6795 | −1.12 | 0.6766 | −1.33 | 0.08 |
| H11_HUMAN | 0.4415 | −1.39 | 0.3741 | 1.09 | 0.7503 |
| H12_HUMAN | 0.945 | −1.10 | 0.5358 | −1.07 | 0.53 |
| H1T_HUMAN | 0.8294 | 1.44 | 0.0262 | 1.59 | 8.0526E−06 |
| H2A1B_HUMAN | 0.9432 | 1.58 | 0.1985 | 1.40 | 0.409 |
| H2A2A_HUMAN | 0.5264 | −1.11 | 0.6082 | 1.09 | 0.5893 |
| H2AY_HUMAN | 0.7845 | −1.17 | 0.3404 | −1.05 | 0.5504 |
| H2AZ_HUMAN | 0.8077 | 1.17 | 0.9585 | 1.59 | 0.4906 |
| H2B1M_HUMAN | 0.6795 | −1.12 | 0.4122 | 1.01 | 0.9475 |
| H2B2C_HUMAN | 0.8696 | 1.20 | 0.0451 | 1.26 | 0.0118 |
| H2B3B_HUMAN | 0.9715 | −1.24 | 0.5341 | −1.29 | 0.4086 |
| H31_HUMAN | 0.7858 | 1.01 | 0.9014 | 1.22 | 0.3426 |
| H31T_HUMAN | 0.8879 | −1.08 | 0.8024 | −1.23 | 0.5046 |
| H32_HUMAN | 0.9355 | 1.20 | 0.7056 | 1.27 | 0.5501 |
| H33_HUMAN | 0.415 | −1.10 | 0.4899 | −1.00 | 0.9733 |
| H3L_HUMAN | 0.7135 | −1.25 | 0.4097 | −1.97 | 0.0608 |
| H4_HUMAN | 0.6795 | 1.05 | 0.8402 | 1.37 | 0.0147 |
| HBA_HUMAN | 0.8696 | 3.70 | 0.0255 | 3.10 | 0.00025709 |
| HBB_HUMAN | 0.6795 | 3.18 | 0.0101 | 2.31 | 0.00011672 |
| HBD_HUMAN | 0.9355 | −1.25 | 0.4175 | −1.16 | 0.627 |
| HBE_HUMAN | 0.6947 | 1.03 | 0.891 | 1.19 | 0.413 |
| HBG1_HUMAN | 0.9849 | 1.84 | 0.0973 | 1.85 | 0.0464 |
| HCDH_HUMAN | 0.6511 | −1.23 | 0.6738 | 1.27 | 0.3593 |
| HEBP2_HUMAN | 0.8696 | −1.09 | 0.7456 | 1.03 | 0.8501 |
| HEMO_HUMAN | 0.2626 | 1.51 | 0.0622 | 1.20 | 0.4982 |
| HMGB2_HUMAN | 0.6795 | −1.11 | 0.7056 | −1.29 | 0.1064 |
| HNRPC_HUMAN | 0.6833 | −1.06 | 0.8024 | −1.22 | 0.1576 |
| HNRPD_HUMAN | 0.6795 | −1.16 | 0.045 | −1.09 | 0.0464 |
| HP1B3_HUMAN | 0.945 | −1.18 | 0.2797 | −1.21 | 0.1298 |
| HPT_HUMAN | 0.8879 | 1.72 | 0.1415 | 1.42 | 0.409 |
| HRG_HUMAN | 0.9144 | 1.65 | 0.0132 | 1.56 | 0.0217 |
| HS90A_HUMAN | 0.945 | −1.07 | 0.8024 | −1.02 | 0.8654 |
| HS90B_HUMAN | 0.9319 | −1.15 | 0.6588 | −1.08 | 0.7662 |
| HSP76_HUMAN | 0.9355 | −1.26 | 0.4743 | −1.18 | 0.5957 |
| HSPB1_HUMAN | 0.6795 | 1.06 | 0.5118 | −1.04 | 0.7432 |
| HSPB2_HUMAN | 0.9639 | −1.14 | 0.6746 | −1.20 | 0.5504 |
| HSPB7_HUMAN | 0.3444 | −1.15 | 0.4175 | −1.33 | 0.0108 |
| IC1_HUMAN | 0.7445 | 1.45 | 0.1415 | 1.19 | 0.4349 |
| ICAL_HUMAN | 0.3444 | 1.06 | 0.8083 | −1.31 | 0.0761 |
| IDHP_HUMAN | 0.8879 | −1.18 | 0.5653 | −1.26 | 0.2523 |
| IF4H_HUMAN | 0.9855 | −1.14 | 0.4376 | −1.14 | 0.3462 |
| IF5A1_HUMAN | 0.7619 | −1.25 | 0.1302 | −1.35 | 0.00028676 |
| IGHA1_HUMAN | 0.9117 | 3.61 | 0.013 | 3.35 | 0.000080479 |
| IGHA2_HUMAN | 0.7398 | 7.94 | 7.4958E−06 | 4.85 | 0.00025709 |
| IGHG1_HUMAN | 0.6795 | 1.46 | 0.3261 | 1.77 | 0.058 |
| IGHG2_HUMAN | 0.6232 | 1.62 | 0.0941 | 2.01 | 0.0113 |
| IGHG3_HUMAN | 0.6795 | −1.24 | 0.0381 | −1.48 | 0.0045 |
| IGHG4_HUMAN | 0.3921 | 1.15 | 0.2344 | 1.84 | 0.019 |
| IGHM_HUMAN | 0.9639 | 4.98 | 0.0031 | 4.62 | 0.0012 |
| IGKC_HUMAN | 0.9438 | 1.83 | 0.1402 | 1.91 | 0.0646 |
| IPYR2_HUMAN | 0.7116 | −1.16 | 0.6441 | −1.47 | 0.1718 |
| ITB1_HUMAN | 0.9355 | −1.18 | 0.5857 | −1.22 | 0.064 |
| ITIH1_HUMAN | 0.0228 | 1.22 | 0.5359 | −1.35 | 0.3286 |
| ITPR3_HUMAN | 0.7845 | 1.02 | 0.84 | 1.11 | 0.4681 |
| K0406_HUMAN | 0.6795 | 1.78 | 0.0826 | 1.32 | 0.5081 |
| KAD1_HUMAN | 0.2107 | −1.26 | 0.036 | −1.12 | 0.2405 |
| KCRB_HUMAN | 0.8077 | −1.31 | 0.3448 | −1.47 | 0.0217 |
| KCRM_HUMAN | 0.6795 | −1.41 | 0.2813 | −1.73 | 0.0045 |
| KCRS_HUMAN | 0.9715 | −1.28 | 0.4773 | −1.24 | 0.2599 |
| KCY_HUMAN | 0.9319 | −1.18 | 0.2564 | −1.14 | 0.3458 |
| KLOTB_HUMAN | 0.8879 | 1.18 | 0.6441 | 1.30 | 0.2784 |
| KNG1_HUMAN | 0.726 | 1.67 | 0.0042 | 1.44 | 0.1063 |
| LAC_HUMAN | 0.8057 | 1.94 | 0.036 | 2.29 | 0.00033751 |
| LAMA2_HUMAN | 0.6795 | −1.18 | 0.3266 | −1.50 | 0.0208 |
| LAMA4_HUMAN | 0.9154 | −1.02 | 0.84 | −1.10 | 0.6148 |
| LAMB1_HUMAN | 0.6795 | −1.21 | 0.4097 | −1.55 | 0.0118 |
| LAMB2_HUMAN | 0.9585 | −1.19 | 0.0126 | −1.23 | 0.1081 |
| LAMC1_HUMAN | 0.7321 | −1.17 | 0.0615 | −1.33 | 0.0253 |
| LAMP1_HUMAN | 0.2626 | 1.06 | 0.8316 | −1.32 | 0.2511 |
| LCORL_HUMAN | 0.9355 | −1.09 | 0.7256 | −1.19 | 0.4389 |
| LDB3_HUMAN | 0.9355 | −1.18 | 0.7823 | −1.21 | 0.3159 |
| LDHB_HUMAN | 0.8879 | −1.14 | 0.6935 | −1.24 | 0.2256 |
| LEG1_HUMAN | 0.6947 | −1.04 | 0.8209 | −1.13 | 0.4383 |
| LG3BP_HUMAN | 0.0472 | −1.14 | 0.4054 | −1.67 | 0.0013 |
| LTBP2_HUMAN | 0.8696 | 2.85 | 0.1191 | 4.12 | 0.0098 |
| LU_HUMAN | 0.8696 | −1.17 | 0.0735 | −1.26 | 0.0464 |
| LUM_HUMAN | 0.9404 | 1.47 | 0.3339 | 1.64 | 0.12 |
| LV202_HUMAN | 0.8879 | −1.68 | 0.0516 | −1.43 | 0.1864 |
| LYSC_HUMAN | 0.945 | −1.23 | 0.732 | −1.35 | 0.6 |
| M3K5_HUMAN | 0.9871 | 1.68 | 0.5435 | 1.64 | 0.3462 |
| M6PBP_HUMAN | 0.9438 | −1.21 | 0.4487 | −1.32 | 0.3986 |
| MAOM_HUMAN | 0.945 | −1.31 | 0.4773 | −1.29 | 0.4875 |
| MARCS_HUMAN | 0.8671 | 1.06 | 0.8316 | −1.10 | 0.6902 |
| MDHC_HUMAN | 0.9355 | −1.20 | 0.6457 | −1.08 | 0.6122 |
| MDHM_HUMAN | 0.9154 | −1.16 | 0.7315 | −1.03 | 0.8761 |
| MFAP4_HUMAN | 0.6795 | 1.63 | 0.1302 | 2.50 | 0.0139 |
| MFAP5_HUMAN | 0.1491 | −1.39 | 0.2767 | 1.45 | 0.0686 |
| MFGM_HUMAN | 0.8696 | −1.00 | 0.9957 | −1.06 | 0.6349 |
| MGST3_HUMAN | 0.4349 | −1.54 | 0.21 | 1.25 | 0.6657 |
| MIME_HUMAN | 0.9715 | 1.47 | 0.3658 | 1.58 | 0.1385 |
| MLE3_HUMAN | 0.472 | −1.05 | 0.7315 | −1.20 | 0.0422 |
| MLL2_HUMAN | 0.6795 | −1.28 | 0.5309 | 1.11 | 0.6182 |
| MLRS_HUMAN | 0.8696 | −1.00 | 0.922 | −1.05 | 0.6431 |
| MLRV_HUMAN | 0.7398 | −1.29 | 0.0028 | −1.15 | 0.2511 |
| MPCP_HUMAN | 0.9639 | −1.35 | 0.388 | −1.30 | 0.244 |
| MRLC2_HUMAN | 0.9871 | 1.24 | 0.3137 | 1.26 | 0.1111 |
| MRLC3_HUMAN | 0.4415 | 1.67 | 0.3658 | −1.23 | 0.733 |
| MSRB2_HUMAN | 0.6795 | 1.02 | 0.9029 | −1.20 | 0.4265 |
| MUC18_HUMAN | 0.3175 | −1.05 | 0.8734 | −1.36 | 0.0686 |
| MYG_HUMAN | 0.2992 | −1.19 | 0.2918 | −1.00 | 0.963 |
| MYH13_HUMAN | 0.8171 | 1.41 | 0.4799 | 1.11 | 0.963 |
| MYH2_HUMAN | 0.8671 | −1.71 | 0.1302 | −2.08 | 0.0045 |
| MYH3_HUMAN | 0.9404 | −1.60 | 0.421 | −1.88 | 0.1734 |
| MYH7_HUMAN | 0.2992 | −1.35 | 0.0246 | −1.14 | 0.2222 |
| MYH8_HUMAN | 0.7451 | −1.30 | 0.4359 | −1.06 | 0.8126 |
| MYL3_HUMAN | 0.472 | −1.25 | 0.0019 | −1.12 | 0.1399 |
| MYL4_HUMAN | 0.8696 | 1.59 | 0.0025 | 1.68 | 0.0051 |
| MYL6_HUMAN | 0.6795 | −1.03 | 0.9585 | 1.28 | 0.2352 |
| MYL9_HUMAN | 0.9404 | −1.02 | 0.9121 | 1.02 | 0.8707 |
| MYLPL_HUMAN | 0.8696 | −1.20 | 0.2434 | −1.11 | 0.5247 |
| MYO6_HUMAN | 0.6795 | 1.31 | 0.4815 | 1.69 | 0.0724 |
| MYOZ2_HUMAN | 0.9585 | −1.16 | 0.4899 | −1.13 | 0.4383 |
| MYP2_HUMAN | 0.6795 | −1.03 | 0.8608 | 1.23 | 0.4875 |
| MYPT1_HUMAN | 0.5264 | 1.03 | 0.8269 | −1.24 | 0.2222 |
| MYPT2_HUMAN | 0.8797 | −1.23 | 0.2564 | −1.39 | 0.1755 |
| NCAM1_HUMAN | 0.6833 | −1.20 | 0.7056 | −1.46 | 0.0587 |
| NDKB_HUMAN | 0.945 | −1.18 | 0.4097 | −1.23 | 0.1662 |
| NDUA4_HUMAN | 0.945 | −1.50 | 0.147 | −1.59 | 0.0493 |
| NDUA7_HUMAN | 0.7147 | −1.49 | 0.0456 | −1.27 | 0.2048 |
| NDUA8_HUMAN | 0.9839 | −1.38 | 0.0469 | −1.42 | 0.1439 |
| NDUAA_HUMAN | 0.9715 | −1.22 | 0.6569 | −1.18 | 0.5504 |
| NDUAC_HUMAN | 0.945 | −1.53 | 0.0084 | −1.61 | 0.0217 |
| NDUAD_HUMAN | 0.7845 | −1.23 | 0.732 | 1.16 | 0.7118 |
| NDUB3_HUMAN | 0.8171 | −1.21 | 0.1197 | −1.34 | 0.1075 |
| NDUB9_HUMAN | 0.9715 | −1.19 | 0.5193 | −1.25 | 0.4265 |
| NDUBA_HUMAN | 0.9715 | −1.37 | 0.0035 | −1.37 | 0.1889 |
| NDUS4_HUMAN | 0.6795 | −1.22 | 0.1302 | −1.48 | 0.0229 |
| NDUS5_HUMAN | 0.8696 | −1.16 | 0.4175 | −1.04 | 0.9299 |
| NDUS6_HUMAN | 0.7398 | −1.33 | 0.0638 | −1.58 | 0.0118 |
| NDUS7_HUMAN | 0.8696 | −1.18 | 0.4508 | −1.27 | 0.1806 |
| NDUS8_HUMAN | 0.9355 | −1.27 | 0.4175 | −1.19 | 0.4681 |
| NDUV1_HUMAN | 0.9432 | −1.22 | 0.5504 | −1.30 | 0.32 |
| NDUV2_HUMAN | 0.9639 | −1.14 | 0.7951 | −1.07 | 0.7395 |
| NDUV3_HUMAN | 0.945 | −1.23 | 0.1257 | −1.30 | 0.2498 |
| NEBL_HUMAN | 0.7135 | −1.23 | 0.5516 | 1.06 | 0.9396 |
| NEXN_HUMAN | 0.7891 | −1.24 | 0.4266 | −1.04 | 0.9396 |
| NID1_HUMAN | 0.945 | 1.72 | 0.00056894 | 1.69 | 0.0004536 |
| NID2_HUMAN | 0.9855 | −1.19 | 0.0328 | −1.20 | 0.2474 |
| NP1L1_HUMAN | 0.9355 | −1.21 | 0.4097 | −1.28 | 0.1445 |
| NP1L4_HUMAN | 0.8671 | −1.02 | 0.9585 | −1.08 | 0.3465 |
| NPM_HUMAN | 0.7891 | −1.13 | 0.464 | −1.21 | 0.0221 |
| NUCL_HUMAN | 0.97 | −1.07 | 0.8048 | −1.08 | 0.5434 |
| OBFC1_HUMAN | 0.7714 | 2.16 | 0.000015322 | 1.92 | 0.000023484 |
| OCAD1_HUMAN | 0.945 | −1.04 | 0.84 | −1.05 | 0.6902 |
| ODPA_HUMAN | 0.9355 | −1.23 | 0.4579 | −1.16 | 0.5046 |
| PABP3_HUMAN | 0.9154 | −1.21 | 0.7056 | −1.06 | 0.8889 |
| PAL4B_HUMAN | 0.4407 | −1.18 | 0.0221 | −1.31 | 1.5112E−07 |
| PARC_HUMAN | 0.9154 | −1.08 | 0.7272 | −1.16 | 0.5241 |
| PARK7_HUMAN | 0.6795 | 1.37 | 0.0166 | 1.58 | 2.7459E−07 |
| PCDH9_HUMAN | 0.8879 | −1.14 | 0.452 | −1.06 | 0.7687 |
| PDIA1_HUMAN | 0.9432 | 1.17 | 0.4986 | 1.10 | 0.7333 |
| PDIA3_HUMAN | 0.9875 | 1.08 | 0.7252 | 1.07 | 0.6928 |
| PDIA6_HUMAN | 0.9871 | −1.01 | 0.972 | 1.01 | 0.9413 |
| PDLI1_HUMAN | 0.6795 | −1.08 | 0.7056 | 1.09 | 0.5945 |
| PDLI3_HUMAN | 0.2338 | −1.08 | 0.6967 | 1.33 | 0.0565 |
| PDLI5_HUMAN | 0.9432 | −1.11 | 0.4743 | −1.15 | 0.178 |
| PEBP1_HUMAN | 0.5535 | −1.16 | 0.0084 | −1.25 | 0.00035967 |
| PGAM1_HUMAN | 0.8696 | 1.54 | 0.0016 | 1.42 | 0.0066 |
| PGAM2_HUMAN | 0.7147 | −1.51 | 0.0723 | −1.32 | 0.12 |
| PGBM_HUMAN | 0.5029 | −1.01 | 0.921 | 1.15 | 0.2874 |
| PGK1_HUMAN | 0.9355 | 1.15 | 0.7315 | 1.39 | 0.3159 |
| PGRC2_HUMAN | 0.7451 | −1.15 | 0.2085 | −1.23 | 0.0299 |
| PGS1_HUMAN | 0.8696 | 1.47 | 0.4097 | 1.83 | 0.1069 |
| PGS2_HUMAN | 0.9355 | 1.21 | 0.5529 | 1.32 | 0.2859 |
| PHP14_HUMAN | 0.27 | −1.15 | 0.1961 | −1.44 | 0.0016 |
| PLCF_HUMAN | 0.9922 | −1.36 | 0.4175 | −1.33 | 0.3286 |
| POPD1_HUMAN | 0.8431 | −1.17 | 0.5684 | −1.40 | 0.1458 |
| PPIA_HUMAN | 0.5797 | −1.16 | 0.0132 | −1.23 | 7.7343E−06 |
| PPIF_HUMAN | 0.0257 | −1.22 | 0.1461 | −1.82 | 2.5066E−08 |
| PRDX1_HUMAN | 0.9355 | −1.07 | 0.7456 | −1.02 | 0.9137 |
| PRDX2_HUMAN | 0.9587 | −1.09 | 0.7256 | −1.11 | 0.4257 |
| PRDX3_HUMAN | 0.9117 | −1.19 | 0.4981 | −1.11 | 0.4379 |
| PRELP_HUMAN | 0.8696 | 1.41 | 0.4743 | 1.85 | 0.1357 |
| PROF1_HUMAN | 0.7269 | −1.12 | 0.4815 | 1.02 | 0.8347 |
| PSD7_HUMAN | 0.9585 | −1.27 | 0.3061 | −1.24 | 0.3189 |
| PTGDS_HUMAN | 0.0257 | −1.26 | 0.0646 | −1.92 | 0.000062471 |
| PTN11_HUMAN | 0.6795 | −1.16 | 0.1694 | −1.31 | 0.0013 |
| PTRF_HUMAN | 0.7269 | −1.08 | 0.4899 | −1.02 | 0.9243 |
| PURA_HUMAN | 0.9715 | −1.13 | 0.3094 | −1.15 | 0.2352 |
| QCR6_HUMAN | 0.7858 | −1.36 | 0.0028 | −1.59 | 0.0409 |
| QCR7_HUMAN | 0.9585 | −1.16 | 0.1419 | −1.14 | 0.3202 |
| QIL1_HUMAN | 0.9208 | −1.10 | 0.536 | −1.17 | 0.413 |
| RABE2_HUMAN | 0.9639 | −1.57 | 0.254 | −1.63 | 0.2552 |
| RHG06_HUMAN | 0.2107 | −1.52 | 0.0381 | −2.87 | 7.7343E−06 |
| RL17_HUMAN | 0.8696 | −1.22 | 0.3404 | −1.37 | 0.016 |
| RL18_HUMAN | 0.9639 | −1.03 | 0.9585 | −1.03 | 0.9137 |
| RL22_HUMAN | 0.8696 | −1.17 | 0.4508 | −1.27 | 0.0137 |
| RL23_HUMAN | 0.7845 | −1.13 | 0.3785 | −1.20 | 0.003 |
| RL23A_HUMAN | 0.9715 | −1.07 | 0.7068 | −1.08 | 0.4916 |
| RL24_HUMAN | 0.6795 | −1.15 | 0.476 | −1.34 | 0.0018 |
| RL27A_HUMAN | 0.4498 | −1.08 | 0.6166 | −1.29 | 0.00018784 |
| RL31_HUMAN | 0.8696 | −1.24 | 0.2434 | −1.33 | 0.00032635 |
| RL35_HUMAN | 0.6795 | −1.22 | 0.3061 | −1.50 | 0.00075976 |
| RL6_HUMAN | 0.6795 | −1.16 | 0.4363 | −1.37 | 0.00065973 |
| RL7_HUMAN | 0.8879 | −1.03 | 0.8734 | −1.10 | 0.5928 |
| RLA2_HUMAN | 0.6795 | −1.18 | 0.2688 | −1.36 | 0.001 |
| ROA1_HUMAN | 0.6795 | −1.23 | 0.2098 | −1.07 | 0.6682 |
| ROA2_HUMAN | 0.7451 | −1.02 | 0.9029 | 1.09 | 0.5945 |
| ROA3_HUMAN | 0.7845 | −1.18 | 0.3061 | −1.08 | 0.5263 |
| RPE_HUMAN | 0.8696 | −1.01 | 0.9153 | 1.05 | 0.5356 |
| RRBP1_HUMAN | 0.865 | −1.25 | 0.4856 | 1.03 | 0.9572 |
| RS13_HUMAN | 0.945 | −1.16 | 0.4156 | −1.12 | 0.5081 |
| RS15_HUMAN | 0.9438 | −1.36 | 0.0227 | −1.33 | 0.0074 |
| RS18_HUMAN | 0.8696 | −1.15 | 0.1302 | −1.20 | 0.019 |
| RS24_HUMAN | 0.7845 | −1.09 | 0.8127 | −1.26 | 0.2474 |
| RS25_HUMAN | 0.6795 | −1.07 | 0.7456 | −1.30 | 0.0029 |
| RS6_HUMAN | 0.7279 | −1.09 | 0.4899 | −1.19 | 0.0066 |
| RS8_HUMAN | 0.2626 | −1.16 | 0.1937 | −1.43 | 2.962E−08 |
| RT36_HUMAN | 0.8696 | −1.32 | 0.0028 | −1.47 | 0.0091 |
| S10A1_HUMAN | 0.8696 | −1.71 | 0.0053 | −1.54 | 0.1542 |
| SAA_HUMAN | 0.9802 | −7.32 | 0.00010051 | −6.41 | 0.00017029 |
| SAMP_HUMAN | 0.9355 | −1.25 | 0.3435 | −1.15 | 0.6657 |
| SAP_HUMAN | 0.9355 | −1.24 | 0.4156 | −1.33 | 0.2352 |
| SDPR_HUMAN | 0.6795 | −1.03 | 0.4899 | −1.12 | 0.1169 |
| SEPT7_HUMAN | 0.7451 | 1.00 | 0.9585 | 1.27 | 0.3972 |
| SET_HUMAN | 0.9715 | −1.27 | 0.2862 | −1.27 | 0.0389 |
| SGCB_HUMAN | 0.6795 | −1.05 | 0.7256 | −1.23 | 0.1532 |
| SGCG_HUMAN | 0.9715 | −1.13 | 0.3785 | −1.17 | 0.4792 |
| SH3BG_HUMAN | 0.8057 | −1.21 | 0.3918 | −1.36 | 0.0085 |
| SIAE_HUMAN | 0.6931 | −1.54 | 0.0255 | −1.87 | 0.0073 |
| SODC_HUMAN | 0.6795 | −1.10 | 0.5358 | −1.24 | 0.0191 |
| SODE_HUMAN | 0.9355 | 1.14 | 0.7561 | 1.09 | 0.8702 |
| SODM_HUMAN | 0.3444 | −1.33 | 0.01 | −1.20 | 0.0796 |
| SRBS2_HUMAN | 0.6511 | 1.01 | 0.94 | 1.46 | 0.2048 |
| SRCA_HUMAN | 0.8981 | −1.24 | 0.3061 | −1.38 | 0.1155 |
| SRCH_HUMAN | 0.3288 | 1.02 | 0.8918 | −1.33 | 0.0648 |
| STIM2_HUMAN | 0.5797 | −1.35 | 0.9625 | 10.77 | 0.1411 |
| SUCA_HUMAN | 0.9715 | −1.33 | 0.0833 | −1.35 | 0.027 |
| TAGL_HUMAN | 0.472 | 1.36 | 0.9585 | 4.40 | 0.0377 |
| TBA1B_HUMAN | 0.8077 | 1.01 | 0.9824 | 1.20 | 0.5504 |
| TBB2A_HUMAN | 0.9639 | 1.51 | 0.4097 | 1.67 | 0.1662 |
| TBB2C_HUMAN | 0.8696 | −1.06 | 0.9014 | 1.07 | 0.7716 |
| TBB3_HUMAN | 0.945 | 1.13 | 0.732 | 1.07 | 0.8201 |
| TBB4_HUMAN | 0.8696 | −1.13 | 0.7068 | −1.32 | 0.0937 |
| TBB5_HUMAN | 0.8696 | 1.02 | 0.9585 | 1.12 | 0.5928 |
| TBB6_HUMAN | 0.9639 | −1.13 | 0.732 | −1.07 | 0.5928 |
| TBB8_HUMAN | 0.945 | 1.41 | 0.4284 | 1.54 | 0.0565 |
| TBB8B_HUMAN | 0.6016 | −1.15 | 0.4547 | −1.47 | 0.0567 |
| TCTP_HUMAN | 0.7891 | −1.38 | 0.0525 | −1.48 | 0.0029 |
| TEBP_HUMAN | 0.6232 | −1.10 | 0.6071 | −1.28 | 0.0217 |
| TELT_HUMAN | 0.7891 | −1.51 | 0.00072989 | −1.36 | 0.0225 |
| TFAM_HUMAN | 0.9438 | −1.24 | 0.2688 | −1.35 | 0.2933 |
| TGM7_HUMAN | 0.9922 | 1.56 | 7.4958E−06 | 1.57 | 0.00035967 |
| THIL_HUMAN | 0.9154 | −1.07 | 0.967 | 1.17 | 0.6962 |
| THIM_HUMAN | 0.8324 | −1.36 | 0.2897 | −1.21 | 0.3116 |
| THIO_HUMAN | 0.3444 | −1.01 | 0.9121 | −1.20 | 0.0681 |
| THRB_HUMAN | 0.6795 | 1.63 | 0.0927 | 1.22 | 0.5286 |
| TI21L_HUMAN | 0.5535 | −1.13 | 0.3741 | −1.47 | 0.0356 |
| TINAL_HUMAN | 0.8696 | −1.26 | 0.0048 | −1.20 | 0.1357 |
| TLE3_HUMAN | 0.472 | 2.16 | 0.1797 | −1.03 | 0.8806 |
| TM40L_HUMAN | 0.3398 | 1.80 | 0.1141 | −1.02 | 0.9285 |
| TMEDA_HUMAN | 0.9639 | −1.25 | 0.1007 | −1.27 | 0.1433 |
| TNNC1_HUMAN | 0.7105 | −1.22 | 0.0023 | −1.36 | 0.0024 |
| TNNI3_HUMAN | 0.9129 | −1.13 | 0.0616 | −1.19 | 0.1662 |
| TNNT1_HUMAN | 0.3444 | 1.17 | 0.1461 | −1.08 | 0.6122 |
| TNNT2_HUMAN | 0.5784 | −1.23 | 0.0223 | −1.42 | 0.000011964 |
| TPIS_HUMAN | 0.6795 | −1.22 | 0.2025 | −1.10 | 0.4383 |
| TPM1_HUMAN | 0.5797 | −1.16 | 0.0163 | −1.27 | 0.00024801 |
| TPM2_HUMAN | 0.3398 | −1.23 | 0.0027 | −1.35 | 7.7343E−06 |
| TPM3_HUMAN | 0.6795 | −1.15 | 0.4032 | −1.33 | 0.0089 |
| TPM3L_HUMAN | 0.7135 | −1.15 | 0.7517 | 1.90 | 0.0847 |
| TPM4_HUMAN | 0.8671 | −1.04 | 0.7056 | 1.04 | 0.8168 |
| TPP1_HUMAN | 0.2107 | −1.16 | 0.3061 | −1.66 | 0.00062631 |
| TPPP_HUMAN | 0.9639 | −1.41 | 0.1662 | −1.47 | 0.1301 |
| TRFE_HUMAN | 0.8289 | 1.98 | 0.000027769 | 1.81 | 0.0004536 |
| TTHY_HUMAN | 0.9715 | 1.49 | 0.0638 | 1.53 | 0.0119 |
| UB2L3_HUMAN | 0.8879 | −1.19 | 0.0869 | −1.23 | 0.0332 |
| UBIQ_HUMAN | 0.0228 | −1.20 | 0.000070995 | −1.04 | 0.2523 |
| UCRI_HUMAN | 0.8696 | −1.33 | 0.0203 | −1.40 | 0.0024 |
| VDAC1_HUMAN | 0.9715 | −1.17 | 0.4892 | −1.16 | 0.2171 |
| VDAC2_HUMAN | 0.945 | −1.17 | 0.3298 | −1.21 | 0.0838 |
| VDAC3_HUMAN | 0.9404 | −1.14 | 0.474 | −1.19 | 0.1371 |
| VIME_HUMAN | 0.726 | 1.16 | 0.6925 | 1.54 | 0.1411 |
| VTDB_HUMAN | 0.8879 | 1.58 | 0.000096546 | 1.48 | 0.0191 |
| VTNC_HUMAN | 0.9319 | 1.07 | 0.8204 | 1.01 | 0.9647 |
| WDR46_HUMAN | 0.8696 | −1.31 | 0.575 | −1.08 | 0.8559 |
| YD007_HUMAN | 0.9715 | −1.66 | 0.3448 | −1.59 | 0.0000136 |
| ZA2G_HUMAN | 0.3398 | 1.35 | 0.1937 | 1.05 | 0.8702 |
| ZN350_HUMAN | 0.7887 | 1.01 | 0.9824 | −1.11 | 0.3986 |

TABLE 8
PHOSPHOENRICHED PEPTIDE EXPRESSION PROFILES

| | | | | NIF v IF | | | | NIF v NF |
|---|---|---|---|---|---|---|---|---|
| | | | NIF v IF Fold | p-value | IF v NF Fold | IF v NF p-value | NIF v NF Fold | p-value |
| Primary Protein Name | Protein Description | Modified Peptide Sequence | Change | (ANOVA) | Change | (ANOVA) | Change | (ANOVA) |
| 4EBP2_HUMAN | Eukaryotic translation initiation | TVAISDAAQLPHDYC[160.0307] | 1.37 | 0.5270 | −1.41 | 0.5240 | −1.03 | 0.9480 |
| | factor 4E-binding protein 2 | TTPGGT[181.014]LFS[166.9984] | | | | | | |
| | OS = Homo sapiens GN = EIF4EBP2 | TTPGGTR | | | | | | |
| | PE = 1 SV = 1 | | | | | | | |
| 4EBP2_HUMAN | Eukaryotic translation initiation | TVAISDAAQLPHDYC[160.0307] | 1.33 | 0.5860 | −1.40 | 0.5560 | −1.06 | 0.9470 |
| | factor 4E-binding protein 2 | TTPGGTLFST[181.014]T[181.014] | | | | | | |
| | OS = Homo sapiens GN = EIF4EBP2 | PGGTR | | | | | | |
| | PE = 1 SV = 1 | | | | | | | |
| AAKB2_HUMAN | 5′-AMP-activated protein kinase | DLSSS[166.9984]PPGPYGQE | 1.54 | 0.7260 | −2.50 | 0.3500 | −1.62 | 0.4340 |
| | subunit beta-2 OS = Homo sapiens | M[147.0354]YAFR | | | | | | |
| | GN = PRKAB2 PE = 1 SV = 1 | | | | | | | |
| AAKB2_HUMAN | 5′-AMP-activated protein kinase | DLSSS[166.9984]PPGPYGQE | −1.99 | 0.5210 | −1.08 | 0.8960 | −2.15 | 0.5200 |
| | subunit beta-2 OS = Homo sapiens | MYAFR | | | | | | |
| | GN = PRKAB2 PE = 1 SV = 1 | | | | | | | |
| AAKB2_HUMAN | 5′-AMP-activated protein kinase | S[166.9984]HNDFVAILDLPEG | −1.78 | 0.6020 | 2.54 | 0.3240 | 1.43 | 0.7550 |
| | subunit beta-2 OS = Homo sapiens | EHQYK | | | | | | |
| | GN = PRKAB2 PE = 1 SV = 1 | | | | | | | |
| ABCF1_HUMAN | ATP-binding cassette sub-family F | KLS[166.9984]VPT[181.014]S | 1.66 | 0.1410 | −1.30 | 0.5360 | 1.28 | 0.6940 |
| | member 1 OS = Homo sapiens | [166.9984]DEEDEVPAPKPR | | | | | | |
| | GN = ABCF1 PE = 1 SV = 2 | | | | | | | |
| ABCF1_HUMAN | ATP-binding cassette sub-family F | KLSVPT[181.014]S[166.9984] | 1.65 | 0.5860 | −1.06 | 0.9570 | 1.55 | 0.5970 |
| | member 1 OS = Homo sapiens | DEEDEVPAPKPR | | | | | | |
| | GN = ABCF1 PE = 1 SV = 2 | | | | | | | |
| ABCF1_HUMAN | ATP-binding cassette sub-family F | KLSVPTS[166.9984]DEEDEVP | 1.03 | 0.9990 | −1.28 | 0.7120 | −1.24 | 0.6750 |
| | member 1 OS = Homo sapiens | APKPR | | | | | | |
| | GN = ABCF1 PE = 1 SV = 2 | | | | | | | |
| ABCF1_HUMAN | ATP-binding cassette sub-family F | LSVPT[181.014]S[166.9984]D | 2.14 | 0.2500 | 1.05 | 0.9960 | 2.24 | 0.1920 |
| | member 1 OS = Homo sapiens | EEDEVPAPKPR | | | | | | |
| | GN = ABCF1 PE = 1 SV = 2 | | | | | | | |
| ABCF1_HUMAN | ATP-binding cassette sub-family F | LSVPTS[166.9984]DEEDEVPA | −1.66 | 0.4500 | −1.29 | 0.7530 | −2.13 | 0.1770 |
| | member 1 OS = Homo sapiens | PKPR | | | | | | |
| | GN = ABCF1 PE = 1 SV = 2 | | | | | | | |
| ABLM1_HUMAN | Actin-binding LIM protein 1 | RSS[166.9984]GREEDDEELLR | −1.27 | 0.8080 | 1.41 | 0.8930 | 1.12 | 0.9180 |
| | OS = Homo sapiens GN = ABLIM1 | | | | | | | |
| | PE = 1 SV = 3 | | | | | | | |
| ABLM1_HUMAN | Actin-binding LIM protein 1 | SS[166.9984]GREEDDEELLR | −1.27 | 0.8590 | 1.19 | 0.9150 | −1.06 | 0.9480 |
| | OS = Homo sapiens GN = ABLIM1 | | | | | | | |
| | PE = 1 SV = 3 | | | | | | | |
| ABLM1_HUMAN | Actin-binding LIM protein 1 | STS[166.9984]QGSINSPVYSR | −2.15 | 0.2540 | −1.18 | 0.8060 | −2.55 | 0.0830 |
| | OS = Homo sapiens GN = ABLIM1 | | | | | | | |
| | PE = 1 SV = 3 | | | | | | | |
| ABLM1_HUMAN | Actin-binding LIM protein 1 | TLS[166.9984]PTPSAEGY[243.0297] | −1.04 | 0.9200 | −1.40 | 0.6810 | −1.46 | 0.4040 |
| | OS = Homo sapiens GN = ABLIM1 | QDVR | | | | | | |
| | PE = 1 SV = 3 | | | | | | | |
| ACINU_HUMAN | Apoptotic chromatin condensation | AES[166.9984]PAEKVPEESVLP | 1.01 | 0.9800 | −1.05 | 0.9600 | −1.04 | 0.9890 |
| | inducer in the nucleus OS = Homo | LVQK | | | | | | |
| | sapiens GN = ACIN1 PE = 1 SV = 1 | | | | | | | |
| ACINU_HUMAN | Apoptotic chromatin condensation | KIS[166.9984]VVSATK | −1.26 | 0.8300 | 1.09 | 0.9240 | −1.15 | 0.9040 |
| | inducer in the nucleus OS = Homo | | | | | | | |
| | sapiens GN = ACIN1 PE = 1 SV = 1 | | | | | | | |
| ACINU_HUMAN | Apoptotic chromatin condensation | KSSS[166.9984]ISEEKGDS[166.9984] | −2.15 | 0.0000 | 2.50 | 0.0000 | 1.16 | 0.8960 |
| | inducer in the nucleus OS = Homo | DDEKPR | | | | | | |
| | sapiens GN = ACIN1 PE = 1 SV = 1 | | | | | | | |
| ACINU_HUMAN | Apoptotic chromatin condensation | KSSSISEEKGDS[166.9984]DDE | 2.32 | 0.3870 | 1.24 | 0.8200 | 2.88 | 0.1080 |
| | inducer in the nucleus OS = Homo | KPR | | | | | | |
| | sapiens GN = ACIN1 PE = 1 SV = 1 | | | | | | | |
| ACINU_HUMAN | Apoptotic chromatin condensation | RLS[166.9984]QPESAEK | −2.93 | 0.0230 | 1.94 | 0.6190 | −1.51 | 0.5710 |
| | inducer in the nucleus OS = Homo | | | | | | | |
| | sapiens GN = ACIN1 PE = 1 SV = 1 | | | | | | | |
| ACINU_HUMAN | Apoptotic chromatin condensation | SSSISEEKGDS[166.9984]DDEK | 1.43 | 0.6160 | −1.34 | 0.7710 | 1.07 | 0.8370 |
| | inducer in the nucleus OS = Homo | PR | | | | | | |
| | sapiens GN = ACIN1 PE = 1 SV = 1 | | | | | | | |
| ACINU_HUMAN | Apoptotic chromatin condensation | TAQVPS[166.9984]PPR | 1.84 | 0.6190 | −1.68 | 0.6460 | 1.09 | 0.9020 |
| | inducer in the nucleus OS = Homo | | | | | | | |
| | sapiens GN = ACIN1 PE = 1 SV = 1 | | | | | | | |
| ACM2_HUMAN | Muscarinic acetylcholine receptor | DKKEPVANQDPVS[166.9984] | 1.18 | 0.9050 | 1.17 | 0.9630 | 1.37 | 0.8900 |
| | M2 OS = Homo sapiens GN = CHRM2 | PSLVQGR | | | | | | |
| | PE = 2 SV = 1 | | | | | | | |
| ACM2_HUMAN | Muscarinic acetylcholine receptor | EPVANQDPVSPS[166.9984]LV | −1.29 | 0.9040 | 1.50 | 0.7470 | 1.16 | 0.9160 |
| | M2 OS = Homo sapiens GN = CHRM2 | QGR | | | | | | |
| | PE = 2 SV = 1 | | | | | | | |
| ADA17_HUMAN | ADAM 17 OS = Homo sapiens | S[166.9984]FEDLTDHPVTR | −1.32 | 0.6590 | 1.10 | 0.8630 | −1.20 | 0.7700 |
| | GN = ADAM17 PE = 1 SV = 1 | | | | | | | |
| ADDB_HUMAN | Beta-adducin OS = Homo sapiens | GLS[166.9984]QM[147.0354] | −2.79 | 0.0590 | −1.60 | 0.8010 | −4.46 | 0.0070 |
| | GN = ADD2 PE = 1 SV = 3 | T[181.014]T[181.014]S[166.9984] | | | | | | |
| | | ADTDVDTSKDKTESVTSGP | | | | | | |
| | | M[147.0354]SPEGSPS[166.9984]K | | | | | | |
| AFF4_HUMAN | AF4/FMR2 family member 4 | SSS[166.9984]PGKPQAVSSLN | −1.02 | 0.9060 | −1.27 | 0.7740 | −1.29 | 0.5670 |
| | OS = Homo sapiens GN = AFF4 PE = 1 | SSHSR | | | | | | |
| | SV = 1 | | | | | | | |
| AG2_HUMAN | Protein Ag2 homolog OS = Homo | S[166.9984]TQSLSLQR | 2.25 | 0.1280 | −1.52 | 0.5060 | 1.48 | 0.6800 |
| | sapiens GN = AG2 PE = 1 SV = 2 | | | | | | | |
| AG2_HUMAN | Protein Ag2 homolog OS = Homo | SFLQS[166.9984]LEC[160.0307] | 1.99 | 0.3780 | −2.15 | 0.2930 | −1.08 | 0.9040 |
| | sapiens GN = AG2 PE = 1 SV = 2 | LR | | | | | | |
| AHNK_HUMAN | Neuroblast differentiation- | KGDRS[166.9984]PEPGQTWTR | 1.10 | 0.9390 | 2.14 | 0.0800 | 2.35 | 0.0300 |
| | associated protein AHNAK - Homo | | | | | | | |
| | sapiens (Human) | | | | | | | |
| AHNK_HUMAN | Neuroblast differentiation- | LKS[166.9984]EDGVEGDLGET | −1.07 | 0.9360 | 1.29 | 0.8650 | 1.20 | 0.9070 |
| | associated protein AHNAK - Homo | QSR | | | | | | |
| | sapiens (Human) | | | | | | | |
| AKA12_HUMAN | A-kinase anchor protein 12 | DSEDVSERDS[166.9984]DKE | 2.57 | 0.6120 | −1.57 | 0.8590 | 1.64 | 0.7160 |
| | OS = Homo sapiens GN = AKAP12 | M[147.0354]ATK | | | | | | |
| | PE = 1 SV = 3 | | | | | | | |
| AKA12_HUMAN | A-kinase anchor protein 12 | EGVTPWAS[166.9984]FKK | 4.43 | 0.0030 | −1.02 | 0.9800 | 4.33 | 0.0080 |
| | OS = Homo sapiens GN = AKAP12 | | | | | | | |
| | PE = 1 SV = 3 | | | | | | | |
| AKA12_HUMAN | A-kinase anchor protein 12 | RPS[166.9984]ESDKEDELDK | 1.45 | 0.8360 | 1.10 | 0.9270 | 1.60 | 0.6910 |
| | OS = Homo sapiens GN = AKAP12 | | | | | | | |
| | PE = 1 SV = 3 | | | | | | | |
| AKA12_HUMAN | A-kinase anchor protein 12 | RPS[166.9984]ESDKEDELDKVK | 1.99 | 0.2880 | 1.17 | 0.6330 | 2.34 | 0.0020 |
| | OS = Homo sapiens GN = AKAP12 | | | | | | | |
| | PE = 1 SV = 3 | | | | | | | |
| AKA12_HUMAN | A-kinase anchor protein 12 | SPPS[166.9984]PVER | 2.15 | 0.1500 | −1.41 | 0.6560 | 1.53 | 0.7880 |
| | OS = Homo sapiens GN = AKAP12 | | | | | | | |
| | PE = 1 SV = 3 | | | | | | | |
| AKAP2_HUMAN | A-kinase anchor protein 2 | VKPPPS[166.9984]PTTEGPSL | −1.01 | 0.9880 | 1.80 | 0.3880 | 1.79 | 0.3250 |
| | OS = Homo sapiens GN = AKAP2 | QPDLAPEEAAGTQRPK | | | | | | |
| | PE = 1 SV = 2 | | | | | | | |
| AKTS1_HUMAN | Proline-rich AKT1 substrate 1 | AATAARPPAPPPAPQPPS[166.9984] | 1.12 | 0.7070 | −1.10 | 0.7970 | 1.02 | 0.9960 |
| | OS = Homo sapiens GN = AKT1S1 | PTPS[166.9984]PPRPTL | | | | | | |
| | PE = 1 SV = 1 | AR | | | | | | |
| AKTS1_HUMAN | Proline-rich AKT1 substrate 1 | S[166.9984]LPVSVPVWGFK | 4.67 | 0.0530 | −2.18 | 0.7040 | 2.14 | 0.5270 |
| | OS = Homo sapiens GN = AKT1S1 | | | | | | | |
| | PE = 1 SV = 1 | | | | | | | |
| ALBU_HUMAN | Serum albumin precursor - Homo | TC[160.0307]VADES[166.9984] | 1.27 | 0.7080 | 2.34 | 0.0000 | 2.99 | 0.0000 |
| | sapiens (Human) | AENC[160.0307]DK | | | | | | |
| ALDOA_HUMAN | Fructose-bisphosphate aldolase A - | GILAADES[166.9984]TGSIAK | −1.86 | 0.1240 | 1.00 | 0.8230 | −1.85 | 0.1720 |
| | Homo sapiens (Human) | | | | | | | |
| ALDOA_HUMAN | Fructose-bisphosphate aldolase A - | GILAADESTGS[166.9984]IAK | −1.38 | 0.5020 | 1.02 | 0.8530 | −1.35 | 0.5870 |
| | Homo sapiens (Human) | | | | | | | |
| ALDOA_HUMAN | Fructose-bisphosphate aldolase A - | GILAADESTGS[166.9984]IAKR | 1.43 | 0.9020 | −1.85 | 0.5880 | −1.29 | 0.5980 |
| | Homo sapiens (Human) | | | | | | | |
| ANK1_HUMAN | Ankyrin-1 OS = Homo sapiens | EADAATS[166.9984]FLR | −1.35 | 0.7000 | −1.19 | 0.8590 | −1.61 | 0.4890 |
| | GN = ANK1 PE = 1 SV = 3 | | | | | | | |
| ANKR2_HUMAN | Ankyrin repeat domain-containing | KTS[166.9984]LDLR | 1.32 | 0.8880 | 1.42 | 0.5750 | 1.87 | 0.3710 |
| | protein 2 OS = Homo sapiens | | | | | | | |
| | GN = ANKRD2 PE = 1 SV = 3 | | | | | | | |
| ANT3_HUMAN | Antithrombin-III OS = Homo sapiens | ATEDEGS[166.9984]EQKIPEA | −1.40 | 0.2890 | 2.77 | 0.1150 | 1.98 | 0.5600 |
| | GN = SERPINC1 PE = 1 SV = 1 | TNR | | | | | | |
| ANT3_HUMAN | Antithrombin-III OS = Homo sapiens | KATEDEGS[166.9984]EQKIPE | −1.50 | 0.3350 | 2.50 | 0.2080 | 1.66 | 0.6580 |
| | GN = SERPINC1 PE = 1 SV = 1 | ATNR | | | | | | |
| APOA1_HUMAN | Apolipoprotein A-I OS = Homo | DYVS[166.9984]QFEGS[166.9984] | 1.06 | 0.9200 | 3.63 | 0.0020 | 3.86 | 0.0010 |
| | sapiens GN = APOA1 PE = 1 SV = 1 | ALGK | | | | | | |
| AQP1_HUMAN | Aquaporin-1 OS = Homo sapiens | VWTSGQVEEYDLDADDINS[166.9984] | 2.35 | 0.3460 | −2.11 | 0.4850 | 1.11 | 0.9910 |
| | GN = AQP1 PE = 1 SV = 3 | RVEM[147.0354]KPK | | | | | | |
| ARGL1_HUMAN | Arginine and glutamate-rich | ASS[166.9984]PPDRIDIFGR | 1.42 | 0.7850 | −2.07 | 0.4190 | −1.45 | 0.6620 |
| | protein 1 OS = Homo sapiens | | | | | | | |
| | GN = ARGLU1 PE = 1 SV = 1 | | | | | | | |
| ARHG5_HUMAN | Rho guanine nucleotide exchange | C[160.0307]S[166.9984]HQPI | 1.31 | 0.4840 | −1.55 | 0.2670 | −1.18 | 0.7700 |
| | factor 5 OS = Homo sapiens | SLLGSFLT[181.014]EESPDK | | | | | | |
| | GN = ARHGEFS PE = 1 SV = 2 | | | | | | | |
| AT11C_HUMAN | Probable phospholipid- | M[147.0354]QMVPSLPPAS[166.9984] | 1.16 | 0.8900 | 2.74 | 0.0004 | 3.17 | 0.0000 |
| | transporting ATPase IG OS = Homo | EC[160.0307]AGEEK | | | | | | |
| | sapiens GN = ATP11C PE = 1 SV = 3 | RVGTR | | | | | | |
| B4GT5_HUMAN | Beta-1,4-galactosyltransferase 5 | VQNAGYSVSRPEGDT[181.014] | 1.59 | 0.5790 | −1.41 | 0.7980 | 1.13 | 0.7390 |
| | OS = Homo sapiens GN = B4GALT5 | GK | | | | | | |
| | PE = 2 SV = 1 | | | | | | | |
| BAG3_HUMAN | BAG family molecular chaperone | SSVQGASS[166.9984]REGS[166.9984] | 6.71 | 0.0030 | −1.68 | 0.7430 | 4.00 | 0.0540 |
| | regulator 3 OS = Homo sapiens | PAR | | | | | | |
| | GN = BAG3 PE = 1 SV = 3 | | | | | | | |
| BAG3_HUMAN | BAG family molecular chaperone | VPPAPVPC[160.0307]PPPS[166.9984] | 1.33 | 0.7340 | 1.37 | 0.6810 | 1.82 | 0.3180 |
| | regulator 3 OS = Homo sapiens | PGPSAVPSSPK | | | | | | |
| | GN = BAG3 PE = 1 SV = 3 | | | | | | | |
| BAG4_HUMAN | BAG family molecular chaperone | ELLELDSVETGGQDS[166.9984] | 1.43 | 0.8750 | 1.70 | 0.7640 | 2.44 | 0.5910 |
| | regulator 4 OS = Homo sapiens | VR | | | | | | |
| | GN = BAG4 PE = 1 SV = 1 | | | | | | | |
| BASI_HUMAN | Basigin precursor - Homo sapiens | KPEDVLDDDDAGSAPLKSS[166.9984] | −7.27 | 0.0000 | 4.62 | 0.0005 | −1.57 | 0.6750 |
| | (Human) | GQHQNDK | | | | | | |
| BASI_HUMAN | Basigin precursor - Homo sapiens | RKPEDVLDDDDAGS[166.9984] | 1.27 | 0.6110 | −1.66 | 0.2240 | −1.31 | 0.7280 |
| | (Human) | APLK | | | | | | |
| BASI_HUMAN | Basigin precursor - Homo sapiens | RKPEDVLDDDDAGSAPLKS[166.9984] | −5.36 | 0.0002 | 2.48 | 0.0590 | −2.16 | 0.5710 |
| | (Human) | SGQHQNDK | | | | | | |
| BASI_HUMAN | Basigin precursor - Homo sapiens | RKPEDVLDDDDAGSAPLKSS[166.9984] | −4.36 | 0.0360 | 2.56 | 0.0940 | −1.70 | 0.8570 |
| | (Human) | GQHQNDK | | | | | | |
| BCLF1_HUMAN | Bcl-2-associated transcription | FNDS[166.9984]EGDDTEETED | 1.30 | 0.7070 | −1.36 | 0.6460 | −1.05 | 0.9110 |
| | factor 1 OS = Homo sapiens | YR | | | | | | |
| | GN = BCLAF1 PE = 1 SV = 2 | | | | | | | |
| BCLF1_HUMAN | Bcl-2-associated transcription | IDIS[166.9984]PSTLR | 1.47 | 0.4350 | −1.76 | 0.1490 | −1.19 | 0.6730 |
| | factor 1 OS = Homo sapiens | | | | | | | |
| | GN = BCLAF1 PE = 1 SV = 2 | | | | | | | |
| BCLF1_HUMAN | Bcl-2-associated transcription | KAEGEPQEES[166.9984]PLK | 1.51 | 0.2470 | −1.55 | 0.2170 | −1.02 | 0.8770 |
| | factor 1 OS = Homo sapiens | | | | | | | |
| | GN = BCLAF1 PE = 1 SV = 2 | | | | | | | |
| BCLF1_HUMAN | Bcl-2-associated transcription | KAEGEPQEES[166.9984]PLKSK | 1.30 | 0.9610 | −1.71 | 0.5860 | −1.32 | 0.3620 |
| | factor 1 OS = Homo sapiens | | | | | | | |
| | GN = BCLAF1 PE = 1 SV = 2 | | | | | | | |
| BCLF1_HUMAN | Bcl-2-associated transcription | LKDLFDYS[166.9984]PPLHK | 2.05 | 0.1510 | −2.54 | 0.0380 | −1.24 | 0.7030 |
| | factor 1 OS = Homo sapiens | | | | | | | |
| | GN = BCLAF1 PE = 1 SV = 2 | | | | | | | |
| BCLF1_HUMAN | Bcl-2-associated transcription | RIDIS[166.9984]PSTLR | 1.69 | 0.3450 | −1.73 | 0.2730 | −1.02 | 0.9700 |
| | factor 1 OS = Homo sapiens | | | | | | | |
| | GN = BCLAF1 PE = 1 SV = 2 | | | | | | | |
| BCLF1_HUMAN | Bcl-2-associated transcription | STFREES[166.9984]PLR | 1.89 | 0.4350 | −1.65 | 0.5550 | 1.14 | 0.9060 |
| | factor 1 OS = Homo sapiens | | | | | | | |
| | GN = BCLAF1 PE = 1 SV = 2 | | | | | | | |
| BCLF1_HUMAN | Bcl-2-associated transcription | Y[243.0297]SPSQNS[166.9984] | 1.10 | 0.8950 | −1.06 | 0.9160 | 1.04 | 0.9630 |
| | factor 1 OS = Homo sapiens | PIHHIPSR | | | | | | |
| | GN = BCLAF1 PE = 1 SV = 2 | | | | | | | |
| BCLF1_HUMAN | Bcl-2-associated transcription | YS[166.9984]PSQNS[166.9984] | 1.35 | 0.6160 | −1.51 | 0.3880 | −1.12 | 0.9020 |
| | factor 1 OS = Homo sapiens | PIHHIPSR | | | | | | |
| | GN = BCLAF1 PE = 1 SV = 2 | | | | | | | |
| BNI3L_HUMAN | BCL2/adenovirus E1B 19 kDa | DHSSQS[166.9984]EEEVVEGE | 1.14 | 0.8360 | 1.26 | 0.8070 | 1.44 | 0.6540 |
| | protein-interacting protein 3-like | KEVEALKK | | | | | | |
| | OS = Homo sapiens GN = BNIP3L | | | | | | | |
| | PE = 1 SV = 1 | | | | | | | |
| BNIP2_HUMAN | BCL2/adenovirus E1B 19 kDa | KGS[166.9984]ITEYTAAEEK | 1.32 | 0.7050 | 1.40 | 0.7030 | 1.85 | 0.3180 |
| | protein-interacting protein 2 | | | | | | | |
| | OS = Homo sapiens GN = BNIP2 PE = 1 | | | | | | | |
| | SV = 1 | | | | | | | |
| BORG4_HUMAN | Cdc42 effector protein 4 OS = Homo | AGPDLPSLPSHALEDEGWAAA | 2.09 | 0.1370 | −1.68 | 0.4770 | 1.24 | 0.8250 |
| | sapiens GN = CDC42EP4 PE = 1 SV = 1 | APS[166.9984]PGSAR | | | | | | |
| CA144_HUMAN | UPF0485 protein C1orf144 | SKS[166.9984]PPKVPIVIQDDS | 1.06 | 0.9370 | 1.07 | 0.9060 | 1.14 | 0.8570 |
| | OS = Homo sapiens GN = C1orf144 | LPAGPPPQIR | | | | | | |
| | PE = 1 SV = 1 | | | | | | | |
| CA144_HUMAN | UPF0485 protein C1orf144 | SKSPPKVPIVIQDDS[166.9984] | −1.06 | 0.9360 | 1.03 | 0.9350 | −1.03 | 0.9900 |
| | OS = Homo sapiens GN = C1orf144 | LPAGPPPQIR | | | | | | |
| | PE = 1 SV = 1 | | | | | | | |
| CA198_HUMAN | Uncharacterized protein C1orf198 | SSS[166.9984]LDALGPTR | 1.60 | 0.5840 | 1.24 | 0.6460 | 1.98 | 0.4410 |
| | OS = Homo sapiens GN = C1orf198 | | | | | | | |
| | PE = 1 SV = 1 | | | | | | | |
| CACB2_HUMAN | Voltage-dependent L-type calcium | SAS[166.9984]QAEEEPSVEPV | −1.54 | 0.5840 | 1.17 | 0.9980 | −1.31 | 0.7090 |
| | channel subunit beta-2 OS = Homo | KK | | | | | | |
| | sapiens GN = CACNB2 PE = 1 SV = 3 | | | | | | | |
| CALD1_HUMAN | Caldesmon OS = Homo sapiens | RGS[166.9984]IGENQVEVM | 1.79 | 0.6360 | −5.25 | 0.0160 | −2.93 | 0.0190 |
| | GN = CALD1 PE = 1 SV = 2 | [147.0354]VEEK | | | | | | |
| CALD1_HUMAN | Caldesmon OS = Homo sapiens | TPDGNKS[166.9984]PAPKPSD | 1.62 | 0.3980 | −1.28 | 0.6030 | 1.26 | 0.9720 |
| | GN = CALD1 PE = 1 SV = 2 | LRPGDVSSK | | | | | | |
| CALX_HUMAN | Calnexin precursor - Homo sapiens | AEEDEILNRS[166.9984]PR | 1.85 | 0.0000 | −1.14 | 0.4660 | 1.62 | 0.0020 |
| | (Human) | | | | | | | |
| CALX_HUMAN | Calnexin precursor - Homo sapiens | SDAEEDGGTVS[166.9984]QE | 2.24 | 0.1340 | −1.95 | 0.2810 | 1.15 | 0.8160 |
| | (Human) | EEDRKPK | | | | | | |
| CAPZB_HUMAN | F-actin-capping protein subunit | ELS[166.9984]QVLTQR | −2.24 | 0.0060 | 1.27 | 0.7640 | −1.76 | 0.1420 |
| | beta - Homo sapiens (Human) | | | | | | | |
| CAR11_HUMAN | Caspase recruitment domain- | S[166.9984]S[166.9984]M[147.0354] | 1.18 | 0.9190 | 1.37 | 0.7040 | 1.63 | 0.4960 |
| | containing protein 11 OS = Homo | S[166.9984]ITAEPPG | | | | | | |
| | sapiens GN = CARD11 PE = 2 SV = 3 | NDSIVR | | | | | | |
| CASA1_BOVIN | Alpha-S1-casein precursor | DIGS[166.9984]ES[166.9984] | 1.79 | 0.6710 | −1.20 | 0.9400 | 1.49 | 0.7390 |
| | [Contains: Antioxidant peptide]- | TEDQAM[147.0354]EDIK | | | | | | |
| | Bos taurus (Bovine) | | | | | | | |
| CASA1_BOVIN | Alpha-S1-casein precursor | DIGS[166.9984]ES[166.9984] | −1.67 | 0.7950 | 1.46 | 0.9150 | −1.14 | 0.9270 |
| | [Contains: Antioxidant peptide]- | TEDQAMEDIK | | | | | | |
| | Bos taurus (Bovine) | | | | | | | |
| CASA1_BOVIN | Alpha-S1-casein precursor | DIGSES[166.9984]T[181.014] | 1.64 | 0.6080 | −1.03 | 0.9600 | 1.59 | 0.7200 |
| | [Contains: Antioxidant peptide]- | EDQAM[147.0354]EDIK | | | | | | |
| | Bos taurus (Bovine) | | | | | | | |
| CASA1_BOVIN | Alpha-S1-casein precursor | DIGSES[166.9984]T[181.014] | −1.44 | 0.8940 | 1.42 | 0.9150 | −1.02 | 0.9830 |
| | [Contains: Antioxidant peptide]- | EDQAMEDIK | | | | | | |
| | Bos taurus (Bovine) | | | | | | | |
| CASA1_BOVIN | Alpha-S1-casein precursor | VPQLEIVPNS[166.9984]AEER | 1.30 | 0.0350 | −1.20 | 0.3600 | 1.08 | 0.8840 |
| | [Contains: Antioxidant peptide]- | | | | | | | |
| | Bos taurus (Bovine) | | | | | | | |
| CASA1_BOVIN | Alpha-S1-casein precursor | YKVPQLEIVPNS[166.9984]AE | 1.31 | 0.2170 | −1.09 | 0.3150 | 1.20 | 0.8490 |
| | [Contains: Antioxidant peptide]- | ER | | | | | | |
| | Bos taurus (Bovine) | | | | | | | |
| CASA2_BOVIN | Alpha-S2-casein precursor | EQLS[166.9984]TS[166.9984] | 1.21 | 0.9350 | 1.50 | 0.6470 | 1.81 | 0.4450 |
| | [Contains: Casocidin-1 - Bos taurus | EENSKK | | | | | | |
| | (Bovine) | | | | | | | |
| CASA2_BOVIN | Alpha-S2-casein precursor | EQLSTS[166.9984]EENSK | 1.21 | 0.8910 | 1.10 | 0.9410 | 1.33 | 0.8200 |
| | [Contains: Casocidin-1 - Bos taurus | | | | | | | |
| | (Bovine) | | | | | | | |
| CASA2_BOVIN | Alpha-S2-casein precursor | NM[147.0354]AINPS[166.9984] | 1.52 | 0.9200 | −1.02 | 0.9500 | 1.48 | 0.8370 |
| | [Contains: Casocidin-1 - Bos taurus | KENLC[160.0307]STFC[160.0307]K | | | | | | |
| | (Bovine) | | | | | | | |
| CASA2_BOVIN | Alpha-S2-casein precursor | NMAINPS[166.9984]KENLC[160.0307] | −1.97 | 0.7830 | 1.58 | 0.9160 | −1.25 | 0.9190 |
| | [Contains: Casocidin-1 - Bos taurus | STFC[160.0307]K | | | | | | |
| | (Bovine) | | | | | | | |
| CASA2_BOVIN | Alpha-S2-casein precursor | TVDM[147.0354]ES[166.9984] | 2.21 | 0.7950 | 1.09 | 0.9060 | 2.41 | 0.6040 |
| | [Contains: Casocidin-1 - Bos taurus | TEVFTK | | | | | | |
| | (Bovine) | | | | | | | |
| CASA2_BOVIN | Alpha-S2-casein precursor | TVDMES[166.9984]TEVFTK | −1.64 | 0.8940 | 1.65 | 0.8160 | 1.01 | 0.9620 |
| | [Contains: Casocidin-1 - Bos taurus | | | | | | | |
| | (Bovine) | | | | | | | |
| CASQ2_HUMAN | Calsequestrin-2 OS = Homo sapiens | KYDLLC[160.0307]LYYHEPVS | −2.48 | 0.2410 | −1.55 | 0.4020 | −3.84 | 0.0210 |
| | GN = CASQ2 PE = 1 SV = 2 | [166.9984]SDKVTQK | | | | | | |
| CAV2_HUMAN | Caveolin-2 OS = Homo sapiens | ADVQLFM[147.0354]DDDSY | 2.60 | 0.3190 | −3.06 | 0.1850 | −1.18 | 0.9100 |
| | GN = CAV2 PE = 1 SV = 2 | [243.0297]SHHS[166.9984]GL | | | | | | |
| | | EYADPEK | | | | | | |
| CBAA1_HUMAN | Calcium-binding atopy-related | MFRLNS[166.9984]LSALAELA | −1.64 | 0.5380 | 1.45 | 0.6930 | −1.13 | 0.9270 |
| | autoantigen 1 OS = Homo sapiens | VGSR | | | | | | |
| | GN = CBARA1 PE = 1 SV = 1 | | | | | | | |
| CBX1_HUMAN | Chromobox protein homolog 1 - | KADS[166.9984]DSEDKGEESK | 1.11 | 0.9370 | −1.85 | 0.2700 | −1.66 | 0.3290 |
| | Homo sapiens (Human) | PK | | | | | | |
| CC85A_HUMAN | Coiled-coil domain-containing | SAS[166.9984]PEHPQKPR | 1.17 | 0.0040 | −2.72 | 0.0001 | −2.32 | 0.4940 |
| | protein 85A OS = Homo sapiens | | | | | | | |
| | GN = CCDC85A PE = 2 SV = 2 | | | | | | | |
| CCD18_HUMAN | Coiled-coil domain-containing | LEQSQKM[147.0354]VIEKEQS | 2.24 | 0.8930 | −5.88 | 0.3170 | −2.63 | 0.3240 |
| | protein 18 OS = Homo sapiens | [166.9984]LQES[166.9984]K | | | | | | |
| | GN = CCDC18 PE = 1 SV = 1 | | | | | | | |
| CCD86_HUMAN | Coiled-coil domain-containing | LGGLRPES[166.9984]PESLTSV | 2.02 | 0.2790 | −1.76 | 0.4800 | 1.15 | 0.8840 |
| | protein 86 OS = Homo sapiens | SR | | | | | | |
| | GN = CCDC86 PE = 1 SV = 1 | | | | | | | |
| CCNY_HUMAN | Cyclin-Y OS = Homo sapiens | SAS[166.9984]ADNLTLPR | 1.36 | 0.7100 | 1.29 | 0.8020 | 1.76 | 0.4740 |
| | GN = CCNY PE = 1 SV = 2 | | | | | | | |
| CD44_HUMAN | CD44 antigen precursor - Homo | KPSGLNGEASKS[166.9984]QE | −1.68 | 0.6920 | 1.58 | 0.7560 | −1.06 | 0.9670 |
| | sapiens (Human) | MVHLVNK | | | | | | |
| CDN1B_HUMAN | Cyclin-dependent kinase inhibitor | VSNGS[166.9984]PSLER | −1.23 | 0.9150 | −1.13 | 0.6820 | −1.38 | 0.6410 |
| | 1B OS = Homo sapiens GN = CDKN1B | | | | | | | |
| | PE = 1 SV = 1 | | | | | | | |
| CE042_HUMAN | Uncharacterized protein C5orf42 | KT[181.014]LAS[166.9984]KT | 1.41 | 0.2060 | −1.41 | 0.0830 | −1.00 | 0.8040 |
| | OS = Homo sapiens GN = C5orf42 | ISIS[166.9984]EEVR | | | | | | |
| | PE = 2 SV = 2 | | | | | | | |
| CE110_HUMAN | Centrosomal protein of 110 kDa | S[166.9984]FQS[166.9984]EA | −1.09 | 0.9350 | −1.98 | 0.0003 | −2.16 | 0.0020 |
| | OS = Homo sapiens GN = CEP110 | PLKRGIVSAQDAS[166.9984]L | | | | | | |
| | PE = 1 SV = 3 | QER | | | | | | |
| CF142_HUMAN | Uncharacterized protein C6orf142 | IPEESSDKS[166.9984]PETVNR | −1.13 | 0.8590 | 1.45 | 0.4680 | 1.28 | 0.6620 |
| | OS = Homo sapiens GN = C6orf142 | | | | | | | |
| | PE = 2 SV = 2 | | | | | | | |
| CF142_HUMAN | Uncharacterized protein C6orf142 | YANLSS[166.9984]PTSTVSESQ | 1.54 | 0.5970 | 2.27 | 0.2080 | 3.51 | 0.0040 |
| | OS = Homo sapiens GN = C6orf142 | LTKPGVIRPVPVK | | | | | | |
| | PE = 2 SV = 2 | | | | | | | |
| CF203_HUMAN | Uncharacterized protein C6orf203 | VDEEDSDEES[166.9984]HHD | 2.24 | 0.5680 | −3.56 | 0.2150 | −1.59 | 0.5690 |
| | OS = Homo sapiens GN = C6orf203 | EM[147.0354]SEQEEELEDDPT | | | | | | |
| | PE = 1 SV = 1 | VVK | | | | | | |
| CHD9_HUMAN | Chromodomain-helicase-DNA- | NM[147.0354]AAM[147.0354] | 2.22 | 0.7790 | −4.15 | 0.3300 | −1.87 | 0.4380 |
| | binding protein 9 OS = Homo | FPM[147.0354]LLSGM[147.0354] | | | | | | |
| | sapiens GN = CHD9 PE = 1 SV = 1 | AGLPNLLGMGGLLTKPTE | | | | | | |
| | | SGT[181.014]EDK | | | | | | |
| CHSP1_HUMAN | Calcium-regulated heat stable | ERS[166.9984]PS[166.9984]P | 1.77 | 0.2890 | −1.76 | 0.3080 | 1.01 | 0.9960 |
| | protein 1 - Homo sapiens (Human) | LRGNVVPS[166.9984]PLPTR | | | | | | |
| CHSP1_HUMAN | Calcium-regulated heat stable | ERS[166.9984]PS[166.9984]P | −2.29 | 0.0410 | 1.63 | 0.3630 | −1.41 | 0.5790 |
| | protein 1 - Homo sapiens (Human) | LRGNVVPSPLPTR | | | | | | |
| CHSP1_HUMAN | Calcium-regulated heat stable | GNVVPS[166.9984]PLPTR | 1.87 | 0.2680 | −2.23 | 0.1370 | −1.19 | 0.6940 |
| | protein 1 - Homo sapiens (Human) | | | | | | | |
| CHSP1_HUMAN | Calcium-regulated heat stable | TFS[166.9984]ATVR | 2.19 | 0.0730 | −1.78 | 0.3170 | 1.23 | 0.6760 |
| | protein 1 - Homo sapiens (Human) | | | | | | | |
| CI078_HUMAN | Uncharacterized protein C9orf78 | VGDTEKPEPERS[166.9984]PP | 1.53 | 0.4420 | −1.41 | 0.6260 | 1.09 | 0.8890 |
| | OS = Homo sapiens GN = C9orf78 | NR | | | | | | |
| | PE = 1 SV = 1 | | | | | | | |
| CI139_HUMAN | Uncharacterized protein C9orf139 | GFC[160.0307]PEM[147.0354] | 2.11 | 0.4320 | −1.31 | 0.8410 | 1.62 | 0.4930 |
| | OS = Homo sapiens GN = C9orf139 | GQNESLS[166.9984]EERK | | | | | | |
| | PE = 2 SV = 1 | | | | | | | |
| CK046_HUMAN | Uncharacterized protein C11orf46 | KPES[166.9984]DGRTAKALR | 1.25 | 0.8480 | −1.47 | 0.5780 | −1.18 | 0.7210 |
| | OS = Homo sapiens GN = C11orf46 | | | | | | | |
| | PE = 2 SV = 1 | | | | | | | |
| CKAP5_HUMAN | Cytoskeleton-associated protein 5 | WNFTT[181.014]PRDEY[243.0297] | −1.38 | 0.6670 | 1.37 | 0.5910 | −1.01 | 0.9040 |
| | OS = Homo sapiens GN = CKAP5 | IEQLKT[181.014]QM[147.0354] | | | | | | |
| | PE = 1 SV = 3 | SS[166.9984]C[160.0307] | | | | | | |
| | | VAK | | | | | | |
| CLC14_HUMAN | C-type lectin domain family 14 | KES[166.9984]M[147.0354]G | 1.50 | 0.7290 | −1.61 | 0.6360 | −1.07 | 0.9020 |
| | member A OS = Homo sapiens | PPGLESDPEPAALGSSSAHC[160.0307] | | | | | | |
| | GN = CLEC14A PE = 1 SV = 1 | TNNGVK | | | | | | |
| CLC14_HUMAN | C-type lectin domain family 14 | KES[166.9984]MGPPGLESDP | −1.74 | 0.3610 | 1.68 | 0.5290 | −1.03 | 0.9500 |
| | member A OS = Homo sapiens | EPAALGSSSAHC[160.0307]TN | | | | | | |
| | GN = CLEC14A PE = 1 SV = 1 | NGVK | | | | | | |
| CLIP3_HUMAN | CAP-Gly domain-containing linker | QGLFASVSKISKAVDAPPSS[166.9984] | −1.20 | 0.8630 | −1.12 | 0.8440 | −1.34 | 0.6830 |
| | protein 3 OS = Homo sapiens | VT[181.014]S[166.9984] | | | | | | |
| | GN = CLIP3 PE = 1 SV = 3 | TPR | | | | | | |
| CLUL1_HUMAN | Clusterin-like protein 1 OS = Homo | APDHGGLIS[166.9984]KM[147.0354] | 1.92 | 0.2380 | −1.78 | 0.3550 | 1.08 | 0.9270 |
| | sapiens GN = CLUL1 PE = 2 SV = 1 | LPGQDR | | | | | | |
| CMYA5_HUMAN | Cardiomyopathy-associated | KRNS[166.9984]FESQDVPTNK | −1.54 | 0.6040 | −1.55 | 0.5810 | −2.39 | 0.2030 |
| | protein 5 OS = Homo sapiens | | | | | | | |
| | GN = CMYAS PE = 1 SV = 3 | | | | | | | |
| COBL_HUMAN | Protein cordon-bleu OS = Homo | KSS[166.9984]LGNDETDKEK | 1.76 | 0.3740 | −1.40 | 0.4270 | 1.26 | 0.8160 |
| | sapiens GN = COBL PE = 1 SV = 2 | | | | | | | |
| COBL_HUMAN | Protein cordon-bleu OS = Homo | RAPAPPPPQPPPPS[166.9984] | 1.19 | 0.9040 | 1.26 | 0.7710 | 1.51 | 0.5130 |
| | sapiens GN = COBL PE = 1 SV = 2 | PLIPNR | | | | | | |
| COBL_HUMAN | Protein cordon-bleu OS = Homo | VSLGS[166.9984]QIDLQK | 1.40 | 0.7780 | 1.29 | 0.6730 | 1.80 | 0.2030 |
| | sapiens GN = COBL PE = 1 SV = 2 | | | | | | | |
| CPEB4_HUMAN | Cytoplasmic polyadenylation | GLNGGIT[181.014]PLNSIS[166.9984] | −1.62 | 0.2700 | 1.61 | 0.3480 | −1.01 | 0.9720 |
| | element-binding protein 4 | PLK | | | | | | |
| | OS = Homo sapiens GN = CPEB4 PE = 1 | | | | | | | |
| | SV = 1 | | | | | | | |
| CPZIP_HUMAN | Capz-interacting protein - Homo | AM[147.0354]VS[166.9984]P | 3.39 | 0.1200 | −4.14 | 0.0660 | −1.22 | 0.7270 |
| | sapiens (Human) | FHS[166.9984]PPSTPSSPGVR | | | | | | |
| CPZIP_HUMAN | Capz-interacting protein - Homo | AM[147.0354]VSPFHS[166.9984] | 1.37 | 0.9520 | −1.80 | 0.7760 | −1.31 | 0.7650 |
| | sapiens (Human) | PPSTPSSPGVR | | | | | | |
| CPZIP_HUMAN | Capz-interacting protein - Homo | AMVS[166.9984]PFHS[166.9984] | −1.11 | 0.9350 | 1.50 | 0.7040 | 1.35 | 0.7260 |
| | sapiens (Human) | PPSTPSSPGVR | | | | | | |
| CPZIP_HUMAN | Capz-interacting protein - Homo | AMVSPFHS[166.9984]PPSTPS | −1.50 | 0.7960 | −1.28 | 0.7140 | −1.92 | 0.6180 |
| | sapiens (Human) | SPGVR | | | | | | |
| CPZIP_HUMAN | Capz-interacting protein - Homo | APGS[166.9984]PLSSEGAAGE | 1.19 | 0.7270 | 1.43 | 0.4370 | 1.71 | 0.1720 |
| | sapiens (Human) | GVR | | | | | | |
| CPZIP_HUMAN | Capz-interacting protein - Homo | RSS[166.9984]EEVDGQHPAQ | 1.08 | 0.8750 | −1.39 | 0.4450 | −1.29 | 0.5690 |
| | sapiens (Human) | EEVPESPQTSGPEAENR | | | | | | |
| CPZIP_HUMAN | Capz-interacting protein - Homo | SKAPGS[166.9984]PLSSEGAA | 1.58 | 0.4310 | −1.03 | 0.9180 | 1.53 | 0.0900 |
| | sapiens (Human) | GEGVR | | | | | | |
| CPZIP_HUMAN | Capz-interacting protein - Homo | SQS[166.9984]DC[160.0307] | −2.54 | 0.0350 | 1.62 | 0.5070 | −1.57 | 0.4470 |
| | sapiens (Human) | GELGDFR | | | | | | |
| CPZIP_HUMAN | Capz-interacting protein - Homo | VDLGQNGEEKS[166.9984]PP | 1.14 | 0.9200 | 2.09 | 0.2970 | 2.38 | 0.1370 |
| | sapiens (Human) | NASHPPK | | | | | | |
| CPZIP_HUMAN | Capz-interacting protein - Homo | VKSS[166.9984]PLIEK | 1.75 | 0.3560 | 3.08 | 0.2380 | 5.39 | 0.0220 |
| | sapiens (Human) | | | | | | | |
| CR025_HUMAN | Uncharacterized protein C18orf25 | RDS[166.9984]SESQLASTESD | −1.41 | 0.8250 | 1.05 | 0.7040 | −1.35 | 0.9500 |
| | OS = Homo sapiens GN = C18orf25 | KPTTGR | | | | | | |
| | PE = 1 SV = 2 | | | | | | | |
| CR025_HUMAN | Uncharacterized protein C18orf25 | RDSSES[166.9984]QLASTESD | −1.12 | 0.8900 | −1.28 | 0.8020 | −1.43 | 0.5910 |
| | OS = Homo sapiens GN = C18orf25 | KPTTGR | | | | | | |
| | PE = 1 SV = 2 | | | | | | | |
| CRIP2_HUMAN | Cysteine-rich protein 2 - Homo | ASS[166.9984]VTTFTGEPNTC | −1.61 | 0.0005 | 1.36 | 0.6380 | −1.19 | 0.6820 |
| | sapiens (Human) | [160.0307]PR | | | | | | |
| CRYAB_HUMAN | Alpha-crystallin B chain OS = Homo | RPFFPFHSPS[166.9984]R | −1.64 | 0.6340 | 1.11 | 0.9830 | −1.48 | 0.7400 |
| | sapiens GN = CRYAB PE = 1 SV = 2 | | | | | | | |
| CSDC2_HUMAN | Cold shock domain-containing | DLPS[166.9984]PLPTK | 1.60 | 0.5860 | −1.15 | 0.7630 | 1.39 | 0.9040 |
| | protein C2 OS = Homo sapiens | | | | | | | |
| | GN = CSDC2 PE = 1 SV = 1 | | | | | | | |
| CSDC2_HUMAN | Cold shock domain-containing | DLPS[166.9984]PLPTKR | 2.69 | 0.3730 | −1.77 | 0.6400 | 1.52 | 0.9060 |
| | protein C2 OS = Homo sapiens | | | | | | | |
| | GN = CSDC2 PE = 1 SV = 1 | | | | | | | |
| CSPG2_HUMAN | Versican core protein OS = Homo | TDGQVS[166.9984]GEAIK | 2.31 | 0.1060 | −1.05 | 0.9060 | 2.20 | 0.3630 |
| | sapiens GN = VCAN PE = 1 SV = 3 | | | | | | | |
| CSRP3_HUMAN | Cysteine and glycine-rich protein 3 | FGES[166.9984]EKC[160.0307] | −1.65 | 0.3070 | 1.02 | 0.9980 | −1.62 | 0.2860 |
| | OS = Homo sapiens GN = CSRP3 PE = 1 | PR | | | | | | |
| | SV = 1 | | | | | | | |
| CSRP3_HUMAN | Cysteine and glycine-rich protein 3 | GIGYGQGAGC[160.0307]LST | −1.25 | 0.3370 | −1.04 | 0.9960 | −1.29 | 0.3420 |
| | OS = Homo sapiens GN = CSRP3 PE = 1 | [181.014]DTGEHLGLQFQQSPK | | | | | | |
| | SV = 1 | PAR | | | | | | |
| CSRP3_HUMAN | Cysteine and glycine-rich protein 3 | GIGYGQGAGC[160.0307]LST | −1.13 | 0.7780 | −1.05 | 0.8450 | −1.18 | 0.6620 |
| | OS = Homo sapiens GN = CSRP3 PE = 1 | DTGEHLGLQFQQS[166.9984] | | | | | | |
| | SV = 1 | PKPAR | | | | | | |
| CSRP3_HUMAN | Cysteine and glycine-rich protein 3 | S[166.9984]LESTNVTDKDGEL | −1.12 | 0.9870 | −1.10 | 0.7040 | −1.23 | 0.7650 |
| | OS = Homo sapiens GN = CSRP3 PE = 1 | YC[160.0307]K | | | | | | |
| | SV = 1 | | | | | | | |
| CX026_HUMAN | UPF0368 protein Cxorf26 | GADS[166.9984]GEEKEEGINR | −1.08 | 0.9060 | −1.12 | 0.8790 | −1.21 | 0.7260 |
| | OS = Homo sapiens GN = CXorf26 | | | | | | | |
| | PE = 1 SV = 1 | | | | | | | |
| CX026_HUMAN | UPF0368 protein Cxorf26 | GADS[166.9984]GEEKEEGINR | −1.04 | 0.8920 | −1.16 | 0.8220 | −1.21 | 0.6140 |
| | OS = Homo sapiens GN = CXorf26 | EDK | | | | | | |
| | PE = 1 SV = 1 | | | | | | | |
| CXA1_HUMAN | Gap junction alpha-1 protein | KLAAGHELQPLAIVDQRPSS[166.9984]R | 1.02 | 0.9480 | −1.66 | 0.5710 | −1.63 | 0.3610 |
| | OS = Homo sapiens GN = GJA1 PE = 1 | | | | | | | |
| | SV = 2 | | | | | | | |
| CXA1_HUMAN | Gap junction alpha-1 protein | KLAAGHELQPLAIVDQRPSS[166.9984] | −1.02 | 0.9660 | −3.89 | 0.0020 | −3.96 | 0.0070 |
| | OS = Homo sapiens GN = GJA1 PE = 1 | RAS[166.9984]S[166.9984]R | | | | | | |
| | SV = 2 | | | | | | | |
| CXA1_HUMAN | Gap junction alpha-1 protein | LAAGHELQPLAIVDQRPS[166.9984] | −2.04 | 0.0960 | 1.29 | 0.6770 | −1.58 | 0.4410 |
| | OS = Homo sapiens GN = GJA1 PE = 1 | SR | | | | | | |
| | SV = 2 | | | | | | | |
| CXA1_HUMAN | Gap junction alpha-1 protein | LAAGHELQPLAIVDQRPS[166.9984] | −1.12 | 0.8580 | −1.16 | 0.7040 | −1.29 | 0.6270 |
| | OS = Homo sapiens GN = GJA1 PE = 1 | SRAS[166.9984]S[166.9984] | | | | | | |
| | SV = 2 | RAS[166.9984]S[166.9984] | | | | | | |
| | | RPRPDDLEI | | | | | | |
| CXA1_HUMAN | Gap junction alpha-1 protein | LAAGHELQPLAIVDQRPSS[166.9984] | −1.22 | 0.8850 | −2.31 | 0.0450 | −2.81 | 0.0430 |
| | OS = Homo sapiens GN = GJA1 PE = 1 | RAS[166.9984]S[166.9984]R | | | | | | |
| | SV = 2 | | | | | | | |
| CXA1_HUMAN | Gap junction alpha-1 protein | LVTGDRNNS[166.9984]SC[160.0307]R | −1.81 | 0.9060 | −1.25 | 0.7110 | −2.26 | 0.1170 |
| | OS = Homo sapiens GN = GJA1 PE = 1 | | | | | | | |
| | SV = 2 | | | | | | | |
| CXA1_HUMAN | Gap junction alpha-1 protein | M[147.0354]GQAGS[166.9984] | 1.68 | 0.7990 | −2.51 | 0.5310 | −1.49 | 0.6110 |
| | OS = Homo sapiens GN = GJA1 PE = 1 | TISNS[166.9984]HAQPFDFP | | | | | | |
| | SV = 2 | DDNQNSK | | | | | | |
| CXA1_HUMAN | Gap junction alpha-1 protein | M[147.0354]GQAGSTISNS[166.9984] | 1.62 | 0.7400 | −1.92 | 0.5920 | −1.18 | 0.8080 |
| | OS = Homo sapiens GN = GJA1 PE = 1 | HAQPFDFPDDNQNSK | | | | | | |
| | SV = 2 | | | | | | | |
| CXA1_HUMAN | Gap junction alpha-1 protein | MGQAGS[166.9984]TIS[166.9984] | −2.57 | 0.4350 | −1.13 | 0.5270 | −2.90 | 0.2970 |
| | OS = Homo sapiens GN = GJA1 PE = 1 | NSHAQPFDFPDDNQNSK | | | | | | |
| | SV = 2 | | | | | | | |
| CXA1_HUMAN | Gap junction alpha-1 protein | MGQAGST[181.014]IS[166.9984] | −2.19 | 0.5190 | 1.21 | 0.9600 | −1.82 | 0.6760 |
| | OS = Homo sapiens GN = GJA1 PE = 1 | NSHAQPFDFPDDNQNSK | | | | | | |
| | SV = 2 | | | | | | | |
| CXA1_HUMAN | Gap junction alpha-1 protein | MGQAGSTIS[166.9984]NSHA | −2.23 | 0.6020 | 1.09 | 0.8020 | −2.04 | 0.6200 |
| | OS = Homo sapiens GN = GJA1 PE = 1 | QPFDFPDDNQNSK | | | | | | |
| | SV = 2 | | | | | | | |
| CXA1_HUMAN | Gap junction alpha-1 protein | SDPYHATS[166.9984]GALSPAK | −1.11 | 0.9610 | 1.30 | 0.7920 | 1.17 | 0.8810 |
| | OS = Homo sapiens GN = GJA1 PE = 1 | | | | | | | |
| | SV = 2 | | | | | | | |
| CYC_HUMAN | Cytochrome c - Homo sapiens | KTGQAPGYS[166.9984]YTAA | 1.05 | 0.9360 | −1.41 | 0.6460 | −1.35 | 0.8250 |
| | (Human) | NK | | | | | | |
| CYC_HUMAN | Cytochrome c - Homo sapiens | KTGQAPGYSYT[181.014]AANK | 1.78 | 0.1710 | −1.84 | 0.1340 | −1.03 | 0.9830 |
| | (Human) | | | | | | | |
| CYC_HUMAN | Cytochrome c - Homo sapiens | TGQAPGYS[166.9984]YTAANK | 1.33 | 0.4000 | −2.60 | 0.0020 | −1.95 | 0.7030 |
| | (Human) | | | | | | | |
| DAP1_HUMAN | Death-associated protein 1 | DKDDQEWESPS[166.9984]PP | −1.09 | 0.8750 | −1.01 | 0.9900 | −1.10 | 0.8880 |
| | OS = Homo sapiens GN = DAP PE = 1 | KPTVFISGVIAR | | | | | | |
| | SV = 3 | | | | | | | |
| DESM_HUMAN | Desmin - Homo sapiens (Human) | TFGGAPGFPLGS[166.9984]PL | 1.50 | 0.2480 | −1.18 | 0.8320 | 1.27 | 0.6180 |
| | | SS[166.9984]PVFPR | | | | | | |
| DESM_HUMAN | Desmin - Homo sapiens (Human) | TFGGAPGFPLGS[166.9984]PL | 2.16 | 0.3460 | −1.25 | 0.9690 | 1.72 | 0.1530 |
| | | SSPVFPR | | | | | | |
| DESM_HUMAN | Desmin - Homo sapiens (Human) | TFGGAPGFPLGSPLSS[166.9984] | 1.41 | 0.7730 | 1.82 | 0.3600 | 2.55 | 0.0620 |
| | | PVFPR | | | | | | |
| DESP_HUMAN | Desmoplakin OS = Homo sapiens | SS[166.9984]SFSDTLEESSPIA | 1.06 | 0.9420 | 1.08 | 0.9370 | 1.14 | 0.8880 |
| | GN = DSP PE = 1 SV = 3 | AIFDTENLEK | | | | | | |
| DNJB6_HUMAN | DnaJ homolog subfamily B | HAPHC[160.0307]LS[166.9984] | 1.89 | 0.0030 | −1.46 | 0.0680 | 1.29 | 0.3420 |
| | member 6 OS = Homo sapiens | EEEGEQDRPR | | | | | | |
| | GN = DNAJB6 PE = 1 SV = 2 | | | | | | | |
| DNJCS_HUMAN | DnaJ homolog subfamily C | SLS[166.9984]TSGESLYHVLGL | 1.56 | 0.3730 | −1.89 | 0.1790 | −1.22 | 0.9720 |
| | member 5 - Homo sapiens | DK | | | | | | |
| | (Human) | | | | | | | |
| DOCK8_HUMAN | Dedicator of cytokinesis protein 8 | LPPNY[243.0297]SM[147.0354] | −1.30 | 0.4540 | −1.15 | 0.8180 | −1.50 | 0.3290 |
| | OS = Homo sapiens GN = DOCK8 | HSAEKVPLQNPPIK | | | | | | |
| | PE = 2 SV = 3 | | | | | | | |
| DSG2_HUMAN | Desmoglein-2 OS = Homo sapiens | VVPSFLPVDQGGS[166.9984]L | 1.77 | 0.5550 | −2.81 | 0.1770 | −1.58 | 0.6380 |
| | GN = DSG2 PE = 1 SV = 2 | VGR | | | | | | |
| DSG2_HUMAN | Desmoglein-2 OS = Homo sapiens | WEEHRS[166.9984]LLSGR | 2.33 | 0.1710 | −2.29 | 0.2130 | 1.02 | 0.8730 |
| | GN = DSG2 PE = 1 SV = 2 | | | | | | | |
| DTD1_HUMAN | D-tyrosyl-tRNA(Tyr) deacylase 1 | SASS[166.9984]GAEGDVSSER | 1.21 | 0.9040 | −1.45 | 0.6400 | −1.20 | 0.6830 |
| | OS = Homo sapiens GN = DTD1 PE = 1 | EP | | | | | | |
| | SV = 2 | | | | | | | |
| DTL_HUMAN | Denticleless protein homolog | GLAPSVDFQQSVT[181.014]V | 1.65 | 0.7220 | −3.53 | 0.1770 | −2.13 | 0.1520 |
| | OS = Homo sapiens GN = DTL PE = 1 | VLFQDENT[181.014]LVS[166.9984] | | | | | | |
| | SV = 2 | AGAVDGIIK | | | | | | |
| DUS27_HUMAN | Inactive dual specificity | KVGS[166.9984]ENKEEVVELSK | −1.39 | 0.7730 | 1.36 | 0.8320 | −1.03 | 0.9720 |
| | phosphatase 27 OS = Homo sapiens | | | | | | | |
| | GN = DUSP27 PE = 2 SV = 1 | | | | | | | |
| E41L2_HUMAN | Band 4.1-like protein 2 OS = Homo | S[166.9984]YTLVVAK | 1.56 | 0.6700 | 1.37 | 0.7790 | 2.14 | 0.3970 |
| | sapiens GN = EPB41L2 PE = 1 SV = 1 | | | | | | | |
| EAN57_HUMAN | Protein EAN57 OS = Homo sapiens | S[166.9984]VRDLEHWHGRK | 2.12 | 0.3920 | −1.91 | 0.5830 | 1.11 | 0.8200 |
| | GN = EAN57 PE = 2 SV = 2 | | | | | | | |
| EF1B_HUMAN | Elongation factor 1-beta - Homo | YGPADVEDTTGSGATDSKDDD | 1.08 | 0.9060 | −1.68 | 0.3300 | −1.56 | 0.4000 |
| | sapiens (Human) | DIDLFGS[166.9984]DDEEESEE | | | | | | |
| | | AKR | | | | | | |
| EF1D_HUMAN | Elongation factor 1-delta - Homo | ATAPQTQHVS[166.9984]PMR | −1.28 | 0.9090 | 1.19 | 0.9590 | −1.08 | 0.9510 |
| | sapiens (Human) | | | | | | | |
| EF1D_HUMAN | Elongation factor 1-delta - Homo | KPATPAEDDEDDDIDLFGS[166.9984] | −1.61 | 0.0530 | 1.22 | 0.5760 | −1.32 | 0.4890 |
| | sapiens (Human) | DNEEEDKEAAQLR | | | | | | |
| EF1D_HUMAN | Elongation factor 1-delta - Homo | KPATPAEDDEDDDIDLFGS[166.9984] | 1.60 | 0.2270 | −1.89 | 0.0850 | −1.18 | 0.6460 |
| | sapiens (Human) | DNEEEDKEAAQLREER | | | | | | |
| EIF3G_HUMAN | Eukaryotic translation initiation | GIPLATGDT[181.014]SPEPELL | 1.39 | 0.6180 | −1.52 | 0.5760 | −1.09 | 0.9040 |
| | factor 3 subunit G OS = Homo | PGAPLPPPK | | | | | | |
| | sapiens GN = EIF3G PE = 1 SV = 2 | | | | | | | |
| EIF3G_HUMAN | Eukaryotic translation initiation | GIPLATGDTS[166.9984]PEPEL | 2.00 | 0.3980 | −1.74 | 0.5830 | 1.15 | 0.9040 |
| | factor 3 subunit G OS = Homo | LPGAPLPPPK | | | | | | |
| | sapiens GN = EIF3G PE = 1 SV = 2 | | | | | | | |
| EMD_HUMAN | Emerin OS = Homo sapiens | DSAYQSITHYRPVS[166.9984] | 2.24 | 0.0430 | −1.42 | 0.4650 | 1.58 | 0.4390 |
| | GN = EMD PE = 1 SV = 1 | ASR | | | | | | |
| ESAM_HUMAN | Endothelial cell-selective adhesion | ALRPPHGPPRPGALT[181.014] | 1.41 | 0.4720 | −1.23 | 0.7090 | 1.15 | 0.8840 |
| | molecule OS = Homo sapiens | PTPS[166.9984]LSSQALPSPR | | | | | | |
| | GN = ESAM PE = 1 SV = 1 | | | | | | | |
| ESAM_HUMAN | Endothelial cell-selective adhesion | ALRPPHGPPRPGALTPT[181.014] | 1.35 | 0.5790 | −1.15 | 0.9100 | 1.18 | 0.6830 |
| | molecule OS = Homo sapiens | PS[166.9984]LS[166.9984] | | | | | | |
| | GN = ESAM PE = 1 SV = 1 | SQALPSPR | | | | | | |
| EVL_HUMAN | Ena/VASP-like protein - Homo | SNS[166.9984]VEKPVSSILSR | 1.26 | 0.9200 | −1.23 | 0.8880 | 1.02 | 0.9120 |
| | sapiens (Human) | | | | | | | |
| F10A1_HUMAN | Hsc70-interacting protein - Homo | KVEEDLKADEPS[166.9984]S[166.9984] | −1.54 | 0.2500 | 1.24 | 0.6680 | −1.24 | 0.5950 |
| | sapiens (Human) | EES[166.9984]DLEIDK | | | | | | |
| F122A_HUMAN | Protein FAM122A OS = Homo | RIDFIPVS[166.9984]PAPSPT[181.014]R | 1.42 | 0.6770 | −1.75 | 0.3500 | −1.23 | 0.7250 |
| | sapiens GN = FAM122A PE = 1 SV = 1 | | | | | | | |
| F122A_HUMAN | Protein FAM122A OS = Homo | RNS[166.9984]TTFPSR | 1.11 | 0.9150 | −1.13 | 0.9000 | −1.02 | 0.9890 |
| | sapiens GN = FAM122A PE = 1 SV = 1 | | | | | | | |
| F122A_HUMAN | Protein FAM122A OS = Homo | SNS[166.9984]APLIHGLSDTSP | 1.12 | 0.8880 | −1.39 | 0.6040 | −1.24 | 0.7340 |
| | sapiens GN = FAM122A PE = 1 SV = 1 | VFQAEAPSAR | | | | | | |
| F122B_HUMAN | Protein FAM122B OS = Homo | SSS[166.9984]APLIHGLSDLSQ | −1.02 | 0.9610 | −1.20 | 0.9070 | −1.23 | 0.8640 |
| | sapiens GN = FAM122B PE = 1 SV = 2 | VFQPYTLR | | | | | | |
| F262_HUMAN | 6-phosphofructo-2- | NYS[166.9984]VGSRPLKPLSP | 1.58 | 0.6460 | −1.39 | 0.7430 | 1.13 | 0.9770 |
| | kinase/fructose-2,6-biphosphatase | LR | | | | | | |
| | 2 OS = Homo sapiens GN = PFKFB2 | | | | | | | |
| | PE = 1 SV = 2 | | | | | | | |
| F90AO_HUMAN | Putative protein FAM90A24 | EVPQAAS[166.9984]KTHGLL | 1.28 | 0.9980 | 1.55 | 0.4290 | 1.99 | 0.1980 |
| | OS = Homo sapiens | QAS[166.9984]RPQAQDK | | | | | | |
| | GN = FAM90A24P PE = 5 SV = 1 | | | | | | | |
| FA49A_HUMAN | Protein FAM49A OS = Homo sapiens | FY[243.0297]EFS[166.9984]IR | 1.22 | 0.9060 | −1.60 | 0.6260 | −1.32 | 0.7260 |
| | GN = FAM49A PE = 1 SV = 1 | LEKALQSLLESLTC[160.0307]P | | | | | | |
| | | PY[243.0297]T[181.014]PT[181.014] | | | | | | |
| | | QHLER | | | | | | |
| FA54B_HUMAN | Protein FAM54B OS = Homo sapiens | NAS[166.9984]VPNLR | 2.51 | 0.0010 | −4.67 | 0.0000 | −1.86 | 0.0320 |
| | GN = FAM54B PE = 1 SV = 2 | | | | | | | |
| FBN1_HUMAN | Fibrillin-1 OS = Homo sapiens | GNPEPPVS[166.9984]GEM[147.0354] | 3.80 | 0.0007 | −2.39 | 0.0730 | 1.59 | 0.6420 |
| | GN = FBN1 PE = 1 SV = 1 | DDNSLSPEAC[160.0307] | | | | | | |
| | | YEC[160.0307]K | | | | | | |
| FCGRN_HUMAN | IgG receptor FcRn large subunit | S[166.9984]GLPAPWISLRGDD | −1.09 | 0.9200 | 3.36 | 0.2650 | 3.10 | 0.2200 |
| | p51 OS = Homo sapiens GN = FCGRT | TGVLLPT[181.014]PGEAQDA | | | | | | |
| | PE = 1 SV = 1 | DLK | | | | | | |
| FETUA_HUMAN | Alpha-2-HS-glycoprotein precursor - | C[160.0307]DSSPDS[166.9984] | −2.17 | 0.3720 | 3.48 | 0.1310 | 1.60 | 0.7390 |
| | Homo sapiens (Human) | AEDVR | | | | | | |
| FETUA_HUMAN | Alpha-2-HS-glycoprotein precursor - | C[160.0307]DSSPDS[166.9984] | −2.09 | 0.3930 | 4.22 | 0.0310 | 2.02 | 0.5390 |
| | Homo sapiens (Human) | AEDVRK | | | | | | |
| FETUA_HUMAN | Alpha-2-HS-glycoprotein precursor - | HTFM[147.0354]GVVSLGSPS | 2.41 | 0.9200 | 2.14 | 0.3500 | 5.15 | 0.0590 |
| | Homo sapiens (Human) | [166.9984]GEVSHPR | | | | | | |
| FETUA_HUMAN | Alpha-2-HS-glycoprotein precursor - | HTFMGVVSLGSPS[166.9984] | −2.32 | 0.7770 | 20.09 | 0.0000 | 8.67 | 0.0000 |
| | Homo sapiens (Human) | GEVSHPR | | | | | | |
| FETUA_HUMAN | Alpha-2-HS-glycoprotein precursor - | HTFMGVVSLGSPSGEVS[166.9984] | −1.88 | 0.7590 | 7.95 | 0.0007 | 4.24 | 0.1060 |
| | Homo sapiens (Human) | HPR | | | | | | |
| FGFR3_HUMAN | Fibroblast growth factor receptor 3 | VVGRAAEVPGPEPGQQEQLVF | 1.53 | 0.5780 | −3.84 | 0.0070 | −2.51 | 0.0030 |
| | OS = Homo sapiens GN = FGFR3 PE = 1 | GS[166.9984]GDAVELS[166.9984] | | | | | | |
| | SV = 1 | C[160.0307]PPPGGGPM | | | | | | |
| | | GPTVWVK | | | | | | |
| FHL2_HUMAN | Four and a half LIM domains | YIS[166.9984]FEER | −3.34 | 0.0130 | −1.04 | 0.9210 | −3.49 | 0.0350 |
| | protein 2 OS = Homo sapiens | | | | | | | |
| | GN = FHL2 PE = 1 SV = 3 | | | | | | | |
| FOXA1_HUMAN | Hepatocyte nuclear factor 3-alpha | SY[243.0297]PHAKPPYSYISLIT | −1.57 | 0.2060 | −2.27 | 0.1420 | −3.55 | 0.0000 |
| | OS = Homo sapiens GN = FOXA1 | M[147.0354]AIQRAPSKMLT[181.014] | | | | | | |
| | PE = 1 SV = 1 | LSEIYQWIM[147.0354] | | | | | | |
| | | DLFPY[243.0297]Y[243.0297]R | | | | | | |
| FRIH_HUMAN | Ferritin heavy chain OS = Homo | KMGAPESGLAEYLFDKHTLGDS | −1.39 | 0.7100 | −1.17 | 0.9590 | −1.63 | 0.6690 |
| | sapiens GN = FTH1 PE = 1 SV = 2 | [166.9984]DNES | | | | | | |
| FTCD_HUMAN | Formimidoyltransferase- | AFVGEVGARSAAPGGGSVAAA | −1.75 | 0.0750 | 1.48 | 0.7740 | −1.19 | 0.5040 |
| | cyclodeaminase OS = Homo sapiens | AAAMGAALGS[166.9984]M[147.0354] | | | | | | |
| | GN = FTCD PE = 1 SV = 2 | VGLMTY[243.0297] | | | | | | |
| | | GRR | | | | | | |
| FUND1_HUMAN | FUN14 domain-containing protein | NPPPQDYESDDDS[166.9984] | −1.04 | 0.9660 | −1.22 | 0.8830 | −1.27 | 0.8570 |
| | 1 OS = Homo sapiens GN = FUNDC1 | YEVLDLTEYAR | | | | | | |
| | PE = 1 SV = 1 | | | | | | | |
| G3BP1_HUMAN | Ras GTPase-activating protein- | SSS[166.9984]PAPADIAQTVQ | 1.36 | 0.6860 | −1.31 | 0.8030 | 1.04 | 0.9820 |
| | binding protein 1 OS = Homo | EDLR | | | | | | |
| | sapiens GN = G3BP1 PE = 1 SV = 1 | | | | | | | |
| G3P_HUMAN | Glyceraldehyde-3-phosphate | GALQNIIPAS[166.9984]TGAAK | −1.32 | 0.6330 | −1.35 | 0.7670 | −1.79 | 0.1420 |
| | dehydrogenase - Homo sapiens | | | | | | | |
| | (Human) | | | | | | | |
| G3P_HUMAN | Glyceraldehyde-3-phosphate | IISNASC[160.0307]T[181.014] | −1.27 | 0.8010 | 1.17 | 0.8120 | −1.09 | 0.9510 |
| | dehydrogenase - Homo sapiens | TNC[160.0307]LAPLAK | | | | | | |
| | (Human) | | | | | | | |
| G3P_HUMAN | Glyceraldehyde-3-phosphate | VIHDNFGIVEGLM[147.0354]T | −1.16 | 0.5330 | −1.61 | 0.9160 | −1.86 | 0.3240 |
| | dehydrogenase - Homo sapiens | TVHAITAT[181.014]QK | | | | | | |
| | (Human) | | | | | | | |
| G3P_HUMAN | Glyceraldehyde-3-phosphate | VIHDNFGIVEGLMTTVHAITAT | −1.56 | 0.6670 | 1.78 | 0.4930 | 1.14 | 0.8970 |
| | dehydrogenase - Homo sapiens | [181.014]QK | | | | | | |
| | (Human) | | | | | | | |
| GATA6_HUMAN | Transcription factor GATA-6 | NT[181.014]S[166.9984]PT[181.014] | −2.11 | 0.0490 | −1.11 | 0.9670 | −2.35 | 0.0140 |
| | OS = Homo sapiens GN = GATA6 | TQPTASGAGAPVM[147.0354] | | | | | | |
| | PE = 1 SV = 2 | TGAGESTNPENSELK | | | | | | |
| GBP4_HUMAN | Guanylate-binding protein 4 | KS[166.9984]EQLNK | 2.24 | 0.2420 | −1.29 | 0.7630 | 1.73 | 0.2940 |
| | OS = Homo sapiens GN = GBP4 PE = 1 | | | | | | | |
| | SV = 2 | | | | | | | |
| GGT5_HUMAN | Gamma-glutamyltransferase 5 | SPSSM[147.0354]VPSILINKA | −1.15 | 0.9690 | −1.48 | 0.7960 | −1.71 | 0.7650 |
| | OS = Homo sapiens GN = GGTS PE = 2 | QGS[166.9984]K | | | | | | |
| | SV = 2 | | | | | | | |
| GNPAT_HUMAN | Dihydroxyacetone phosphate | FTSQLLDQGTS[166.9984]QC | 3.49 | 0.1980 | −1.09 | 0.9480 | 3.19 | 0.0280 |
| | acyltransferase OS = Homo sapiens | [160.0307]Y[243.0297]DVLSS | | | | | | |
| | GN = GNPAT PE = 1 SV = 1 | DVQK | | | | | | |
| GPSM1_HUMAN | G-protein-signaling modulator 1 | LDDQRASVGS[166.9984]LPGLR | −2.20 | 0.6610 | −2.23 | 0.5710 | −4.92 | 0.1440 |
| | OS = Homo sapiens GN = GPSM1 | | | | | | | |
| | PE = 1 SV = 1 | | | | | | | |
| GPTC8_HUMAN | G patch domain-containing protein | GPKPEPPGS[166.9984]GSPAP | −1.86 | 0.1480 | 1.92 | 0.1190 | 1.03 | 0.9610 |
| | 8 OS = Homo sapiens GN = GPATCH8 | PR | | | | | | |
| | PE = 1 SV = 1 | | | | | | | |
| GRAM4_HUMAN | GRAM domain-containing protein | RLQT[181.014]TSSRS[166.9984] | −1.34 | 0.8470 | 1.07 | 0.9900 | −1.26 | 0.8450 |
| | 4 OS = Homo sapiens GN = GRAMD4 | Y[243.0297]VPSAPAGLGK | | | | | | |
| | PE = 1 SV = 1 | | | | | | | |
| GSK3A_HUMAN | Glycogen synthase kinase-3 alpha | GEPNVSY[243.0297]IC[160.0307] | 1.53 | 0.5460 | −1.14 | 0.8810 | 1.34 | 0.7090 |
| | OS = Homo sapiens GN = GSK3A | SR | | | | | | |
| | PE = 1 SV = 2 | | | | | | | |
| H12_HUMAN | Histone H1.2 - Homo sapiens | S[166.9984]ETAPAAPAAAPPA | 1.31 | 0.8560 | −1.56 | 0.6000 | −1.20 | 0.7650 |
| | (Human) | EK | | | | | | |
| H31_HUMAN | Histone H3.1 OS = Homo sapiens | ST[181.014]ELLIR | 1.39 | 0.7990 | −2.13 | 0.2930 | −1.53 | 0.6540 |
| | GN = HIST1H3A PE = 1 SV = 2 | | | | | | | |
| HABP4_HUMAN | Intracellular hyaluronan-binding | KSLPAPVAQRPDS[166.9984]P | 2.05 | 0.2840 | −1.46 | 0.6530 | 1.41 | 0.6640 |
| | protein 4 OS = Homo sapiens | GGGLQAPGQK | | | | | | |
| | GN = HABP4 PE = 1 SV = 1 | | | | | | | |
| HABP4_HUMAN | Intracellular hyaluronan-binding | SLPAPVAQRPDS[166.9984]PG | 1.30 | 0.6560 | 1.12 | 0.8700 | 1.45 | 0.4830 |
| | protein 4 OS = Homo sapiens | GGLQAPGQK | | | | | | |
| | GN = HABP4 PE = 1 SV = 1 | | | | | | | |
| HAP28_HUMAN | 28 kDa heat- and acid-stable | KSLDS[166.9984]DES[166.9984] | −1.18 | 0.7460 | −1.11 | 0.7970 | −1.31 | 0.4780 |
| | phosphoprotein OS = Homo sapiens | EDEEDDYQQK | | | | | | |
| | GN = PDAP1 PE = 1 SV = 1 | | | | | | | |
| HAP28_HUMAN | 28 kDa heat- and acid-stable | S[166.9984]LDSDES[166.9984] | 1.03 | 0.9480 | −1.23 | 0.6200 | −1.19 | 0.6760 |
| | phosphoprotein OS = Homo sapiens | EDEEDDYQQK | | | | | | |
| | GN = PDAP1 PE = 1 SV = 1 | | | | | | | |
| HAP28_HUMAN | 28 kDa heat- and acid-stable | SLDS[166.9984]DES[166.9984] | 1.01 | 0.9360 | −1.41 | 0.7040 | −1.39 | 0.4780 |
| | phosphoprotein OS = Homo sapiens | EDEEDDYQQKR | | | | | | |
| | GN = PDAP1 PE = 1 SV = 1 | | | | | | | |
| HDAC2_HUMAN | Histone deacetylase 2 OS = Homo | M[147.0354]LPHAPGVQM[147.0354] | 2.76 | 0.3350 | −2.79 | 0.3500 | −1.01 | 0.9040 |
| | sapiens GN = HDAC2 PE = 1 SV = 2 | QAIPEDAVHEDS[166.9984] | | | | | | |
| | | GDEDGEDPDKR | | | | | | |
| HDAC2_HUMAN | Histone deacetylase 2 OS = Homo | MLPHAPGVQMQAIPEDAVHE | −1.50 | 0.8310 | 1.24 | 0.9150 | −1.21 | 0.9330 |
| | sapiens GN = HDAC2 PE = 1 SV = 2 | DS[166.9984]GDEDGEDPDKR | | | | | | |
| HDGF_HUMAN | Hepatoma-derived growth factor | AGDLLEDS[166.9984]PK | 1.19 | 0.8240 | 1.06 | 0.9780 | 1.26 | 0.7650 |
| | OS = Homo sapiens GN = HDGF PE = 1 | | | | | | | |
| | SV = 1 | | | | | | | |
| HDGF_HUMAN | Hepatoma-derived growth factor | AGDLLEDS[166.9984]PKRPK | 1.61 | 0.1550 | −1.44 | 0.3560 | 1.12 | 0.8860 |
| | OS = Homo sapiens GN = HDGF PE = 1 | | | | | | | |
| | SV = 1 | | | | | | | |
| HDGF_HUMAN | Hepatoma-derived growth factor | GNAEGS[166.9984]S[166.9984] | −1.75 | 0.0160 | 1.10 | 0.9610 | −1.59 | 0.0310 |
| | OS = Homo sapiens GN = HDGF PE = 1 | DEEGKLVIDEPAK | | | | | | |
| | SV = 1 | | | | | | | |
| HDGF_HUMAN | Hepatoma-derived growth factor | GNAEGS[166.9984]S[166.9984] | −1.19 | 0.5200 | 1.02 | 0.9290 | −1.16 | 0.5950 |
| | OS = Homo sapiens GN = HDGF PE = 1 | DEEGKLVIDEPAKEK | | | | | | |
| | SV = 1 | | | | | | | |
| HDGF_HUMAN | Hepatoma-derived growth factor | GNAEGSS[166.9984]DEEGKLV | −1.10 | 0.9030 | −1.05 | 0.8580 | −1.15 | 0.6830 |
| | OS = Homo sapiens GN = HDGF PE = 1 | IDEPAK | | | | | | |
| | SV = 1 | | | | | | | |
| HDGF_HUMAN | Hepatoma-derived growth factor | KGNAEGS[166.9984]S[166.9984] | −1.31 | 0.3970 | 1.04 | 0.9350 | −1.25 | 0.4380 |
| | OS = Homo sapiens GN = HDGF PE = 1 | DEEGKLVIDEPAK | | | | | | |
| | SV = 1 | | | | | | | |
| HDGF_HUMAN | Hepatoma-derived growth factor | NSTPS[166.9984]EPGSGR | −1.37 | 0.0009 | 1.21 | 0.0002 | −1.13 | 0.9340 |
| | OS = Homo sapiens GN = HDGF PE = 1 | | | | | | | |
| | SV = 1 | | | | | | | |
| HDGF_HUMAN | Hepatoma-derived growth factor | RAGDLLEDS[166.9984]PK | 1.64 | 0.4560 | −1.44 | 0.5830 | 1.14 | 0.9890 |
| | OS = Homo sapiens GN = HDGF PE = 1 | | | | | | | |
| | SV = 1 | | | | | | | |
| HDGF_HUMAN | Hepatoma-derived growth factor | RAGDLLEDS[166.9984]PKRPK | 1.65 | 0.5810 | −1.31 | 0.7640 | 1.27 | 0.9390 |
| | OS = Homo sapiens GN = HDGF PE = 1 | | | | | | | |
| | SV = 1 | | | | | | | |
| HDGR2_HUMAN | Hepatoma-derived growth factor- | KRS[166.9984]EGFSMDR | −6.87 | 0.0130 | 2.88 | 0.4310 | −2.39 | 0.5530 |
| | related protein 2 OS = Homo | | | | | | | |
| | sapiens GN = HDGFRP2 PE = 1 SV = 1 | | | | | | | |
| HEAT1_HUMAN | HEAT repeat-containing protein 1 | TVKMVIPALIQS[166.9984]DS | 1.04 | 0.9200 | 1.96 | 0.0530 | 2.04 | 0.0220 |
| | OS = Homo sapiens GN = HEATR1 | [166.9984]GDSIEVS[166.9984]R | | | | | | |
| | PE = 1 SV = 3 | | | | | | | |
| HMGA1_HUMAN | High mobility group protein HMG- | KLEKEEEEGIS[166.9984]QES | 1.09 | 0.9200 | −1.52 | 0.4630 | −1.39 | 0.4180 |
| | I/HMG-Y OS = Homo sapiens | [166.9984]S[166.9984]EEEQ | | | | | | |
| | GN = HMGA1 PE = 1 SV = 3 | | | | | | | |
| HMGA1_HUMAN | High mobility group protein HMG- | KLEKEEEEGISQES[166.9984]S | 1.01 | 0.9710 | −1.37 | 0.6330 | −1.36 | 0.4740 |
| | I/HMG-Y OS = Homo sapiens | [166.9984]EEEQ | | | | | | |
| | GN = HMGA1 PE = 1 SV = 3 | | | | | | | |
| HN1_HUMAN | Hematological and neurological | RNS[166.9984]SEASSGDFLDLK | −1.18 | 0.9200 | −1.20 | 0.8200 | −1.41 | 0.7160 |
| | expressed 1 protein OS = Homo | | | | | | | |
| | sapiens GN = HN1 PE = 1 SV = 3 | | | | | | | |
| HNRL2_HUMAN | Heterogeneous nuclear | REEDEPEERS[166.9984]GDET | 1.88 | 0.1720 | 1.04 | 0.9430 | 1.96 | 0.0940 |
| | ribonucleoprotein U-like protein 2 | PGSEVPGDK | | | | | | |
| | OS = Homo sapiens GN = HNRNPUL2 | | | | | | | |
| | PE = 1 SV = 1 | | | | | | | |
| HNRPD_HUMAN | Heterogeneous nuclear | IDASKNEEDEGHSNS[166.9984] | 1.86 | 0.1530 | −1.43 | 0.4310 | 1.30 | 0.6690 |
| | ribonucleoprotein D0 - Homo | SPR | | | | | | |
| | sapiens (Human) | | | | | | | |
| HNRPD_HUMAN | Heterogeneous nuclear | IDASKNEEDEGHSNSS[166.9984] | 1.60 | 0.5170 | −1.28 | 0.9410 | 1.25 | 0.0980 |
| | ribonucleoprotein D0 - Homo | PR | | | | | | |
| | sapiens (Human) | | | | | | | |
| HNRPG_HUMAN | Heterogeneous nuclear | DVYLS[166.9984]PR | 2.28 | 0.4590 | −1.09 | 0.9300 | 2.09 | 0.1340 |
| | ribonucleoprotein G - Homo | | | | | | | |
| | sapiens (Human) | | | | | | | |
| HNRPU_HUMAN | Heterogeneous nuclear | AKS[166.9984]PQPPVEEEDEH | 1.33 | 0.5480 | −1.14 | 0.8520 | 1.16 | 0.6750 |
| | ribonucleoprotein U - Homo | FDDTVVC[160.0307]LDTYNC | | | | | | |
| | sapiens (Human) | [160.0307]DLHFK | | | | | | |
| HP1B3_HUMAN | Heterochromatin protein 1- | TVNSTRET[181.014]PPK | 1.45 | 0.5790 | −1.52 | 0.4310 | −1.05 | 0.9020 |
| | binding protein 3 - Homo sapiens | | | | | | | |
| | (Human) | | | | | | | |
| HS90A_HUMAN | Heat shock protein HSP 90-alpha - | ES[166.9984]EDKPEIEDVGSD | −3.42 | 0.0002 | 3.74 | 0.0000 | 1.09 | 0.9040 |
| | Homo sapiens (Human) | EEEEKK | | | | | | |
| HS90A_HUMAN | Heat shock protein HSP 90-alpha - | ES[166.9984]EDKPEIEDVGSD | −1.09 | 0.7050 | 1.80 | 0.0860 | 1.65 | 0.3240 |
| | Homo sapiens (Human) | EEEEKKDGDK | | | | | | |
| HS90A_HUMAN | Heat shock protein HSP 90-alpha - | ESEDKPEIEDVGS[166.9984]D | 1.07 | 0.9840 | 1.24 | 0.6810 | 1.33 | 0.5270 |
| | Homo sapiens (Human) | EEEEKKDGDK | | | | | | |
| HS90B_HUMAN | Heat shock protein HSP 90-beta - | IEDVGS[166.9984]DEEDDSGK | 2.71 | 0.0850 | 2.76 | 0.4340 | 7.48 | 0.0060 |
| | Homo sapiens (Human) | | | | | | | |
| HS90B_HUMAN | Heat shock protein HSP 90-beta - | IEDVGS[166.9984]DEEDDSGK | 1.29 | 0.9070 | −1.03 | 0.9940 | 1.25 | 0.8900 |
| | Homo sapiens (Human) | DK | | | | | | |
| HS90B_HUMAN | Heat shock protein HSP 90-beta - | IEDVGS[166.9984]DEEDDSGK | 1.55 | 0.4560 | −1.27 | 0.8130 | 1.22 | 0.7200 |
| | Homo sapiens (Human) | DKK | | | | | | |
| HSPB1_HUMAN | Heat shock protein beta-1 - Homo | GPS[166.9984]WDPFR | −4.01 | 0.0001 | 1.76 | 0.4800 | −2.28 | 0.0720 |
| | sapiens (Human) | | | | | | | |
| HSPB1_HUMAN | Heat shock protein beta-1 - Homo | GPS[166.9984]WDPFRDWYP | −3.32 | 0.0000 | −1.07 | 0.8020 | −3.56 | 0.0001 |
| | sapiens (Human) | HSR | | | | | | |
| HSPB1_HUMAN | Heat shock protein beta-1 - Homo | QLS[166.9984]SGVSEIR | −3.99 | 0.3570 | 4.53 | 0.0008 | 1.14 | 0.6120 |
| | sapiens (Human) | | | | | | | |
| HSPB7_HUMAN | Heat shock protein beta-7 | AERS[166.9984]FHSSSSSSSSS | −2.80 | 0.0880 | 3.83 | 0.0520 | 1.36 | 0.8400 |
| | OS = Homo sapiens GN = HSPB7 | TSSSASR | | | | | | |
| | PE = 1 SV = 1 | | | | | | | |
| HSPB7_HUMAN | Heat shock protein beta-7 | AERSFHSSSSSSSSS[166.9984] | −3.08 | 0.1410 | 5.03 | 0.1040 | 1.63 | 0.8280 |
| | OS = Homo sapiens GN = HSPB7 | TSSSASR | | | | | | |
| | PE = 1 SV = 1 | | | | | | | |
| HSPB7_HUMAN | Heat shock protein beta-7 | S[166.9984]FHSSSSSSSSSTSSS | −3.46 | 0.0060 | 2.83 | 0.0510 | −1.22 | 0.8170 |
| | OS = Homo sapiens GN = HSPB7 | ASR | | | | | | |
| | PE = 1 SV = 1 | | | | | | | |
| HSPB7_HUMAN | Heat shock protein beta-7 | SFHS[166.9984]S[166.9984]S | −3.20 | 0.1480 | 2.51 | 0.3550 | −1.27 | 0.8810 |
| | OS = Homo sapiens GN = HSPB7 | SSSSSSTSSSASR | | | | | | |
| | PE = 1 SV = 1 | | | | | | | |
| HSPB7_HUMAN | Heat shock protein beta-7 | SFHS[166.9984]SSSSSSSSTSSS | −2.56 | 0.0020 | 2.27 | 0.1730 | −1.13 | 0.6930 |
| | OS = Homo sapiens GN = HSPB7 | ASR | | | | | | |
| | PE = 1 SV = 1 | | | | | | | |
| HSPB8_HUMAN | Heat shock protein beta-8 | DPFRDS[166.9984]PLSSR | 5.28 | 0.0000 | −5.87 | 0.0000 | −1.11 | 0.8580 |
| | OS = Homo sapiens GN = HSPB8 | | | | | | | |
| | PE = 1 SV = 1 | | | | | | | |
| HSPB8_HUMAN | Heat shock protein beta-8 | FGVPAEGRT[181.014]PPPFPG | 1.45 | 0.5980 | −1.04 | 0.9980 | 1.39 | 0.6620 |
| | OS = Homo sapiens GN = HSPB8 | EPWK | | | | | | |
| | PE = 1 SV = 1 | | | | | | | |
| HTSF1_HUMAN | HIV Tat-specific factor 1 OS = Homo | DLDEEGS[166.9984]EKELHEN | −1.78 | 0.3930 | 1.54 | 0.6890 | −1.16 | 0.8250 |
| | sapiens GN = HTATSF1 PE = 1 SV = 1 | VLDK | | | | | | |
| HTSF1_HUMAN | HIV Tat-specific factor 1 OS = Homo | ESS[166.9984]PEKEAEEGC[160.0307] | −1.08 | 0.9200 | −1.06 | 0.8020 | −1.14 | 0.7270 |
| | sapiens GN = HTATSF1 PE = 1 SV = 1 | PEKESEEGC[160.0307] | | | | | | |
| | | PK | | | | | | |
| HTSF1_HUMAN | HIV Tat-specific factor 1 OS = Homo | VFDDES[166.9984]DEKEDEEY | −1.06 | 0.9480 | 1.11 | 0.9670 | 1.05 | 0.9830 |
| | sapiens GN = HTATSF1 PE = 1 SV = 1 | ADEK | | | | | | |
| HUG1_HUMAN | Protein HUG-1 OS = Homo sapiens | LRRPET[181.014]S[166.9984] | 1.62 | 0.7270 | −1.90 | 0.6790 | −1.17 | 0.9650 |
| | GN = HUG1 PE = 2 SV = 1 | GISDAGGAR | | | | | | |
| I23O_HUMAN | Indoleamine 2,3-dioxygenase | ENKTS[166.9984]EDPSK | 1.21 | 0.9180 | −1.48 | 0.5850 | −1.22 | 0.6660 |
| | OS = Homo sapiens GN = INDO PE = 1 | | | | | | | |
| | SV = 1 | | | | | | | |
| ICAL_HUMAN | Calpastatin OS = Homo sapiens | EGITGPPADSSKPIGPDDAIDAL | −1.07 | 0.9200 | −1.86 | 0.2940 | −2.00 | 0.6620 |
| | GN = CAST PE = 1 SV = 4 | SSDFTC[160.0307]GS[166.9984] | | | | | | |
| | | PTAAGK | | | | | | |
| ICAL_HUMAN | Calpastatin OS = Homo sapiens | EGITGPPADSSKPIGPDDAIDAL | −1.16 | 0.9710 | −1.63 | 0.3610 | −1.89 | 0.6700 |
| | GN = CAST PE = 1 SV = 4 | SSDFTC[160.0307]GSPT[181.014] | | | | | | |
| | | AAGK | | | | | | |
| ICAL_HUMAN | Calpastatin OS = Homo sapiens | KEGITGPPADSSKPIGPDDAIDA | 1.20 | 0.6660 | −1.54 | 0.4360 | −1.28 | 0.9820 |
| | GN = CAST PE = 1 SV = 4 | LSSDFTC[160.0307]GS[166.9984] | | | | | | |
| | | PTAAGK | | | | | | |
| ICLN_HUMAN | Methylosome subunit pICln | FEEESKEPVADEEEEDS[166.9984] | 1.06 | 0.9200 | −1.42 | 0.4970 | −1.33 | 0.4900 |
| | OS = Homo sapiens GN = CLNS1A | DDDVEPITEFR | | | | | | |
| | PE = 1 SV = 1 | | | | | | | |
| IF2P_HUMAN | Eukaryotic translation initiation | KQS[166.9984]FDDNDS[166.9984] | −1.61 | 0.3720 | −1.15 | 0.8020 | −1.86 | 0.1430 |
| | factor 5B OS = Homo sapiens | EELEDKDSK | | | | | | |
| | GN = EIF5B PE = 1 SV = 3 | | | | | | | |
| IF2P_HUMAN | Eukaryotic translation initiation | NKPGPNIES[166.9984]GNED | −1.19 | 0.5740 | −1.28 | 0.5750 | −1.53 | 0.0750 |
| | factor 5B OS = Homo sapiens | DDASFK | | | | | | |
| | GN = EIF5B PE = 1 SV = 3 | | | | | | | |
| IF2P_HUMAN | Eukaryotic translation initiation | VEMYS[166.9984]GS[166.9984] | −3.89 | 0.0830 | 2.14 | 0.5540 | −1.81 | 0.6760 |
| | factor 5B OS = Homo sapiens | DDDDDFNKLPK | | | | | | |
| | GN = EIF5B PE = 1 SV = 3 | | | | | | | |
| IF2P_HUMAN | Eukaryotic translation initiation | WDGS[166.9984]EEDEDNSKK | 1.10 | 0.9040 | 1.01 | 0.9590 | 1.11 | 0.9380 |
| | factor 5B OS = Homo sapiens | | | | | | | |
| | GN = EIF5B PE = 1 SV = 3 | | | | | | | |
| IF4B_HUMAN | Eukaryotic translation initiation | SLENETLNKEEDC[160.0307]H | 1.16 | 0.6760 | 1.34 | 0.5590 | 1.55 | 0.2940 |
| | factor 4B OS = Homo sapiens | S[166.9984]PTSKPPKPDQPLK | | | | | | |
| | GN = EIF4B PE = 1 SV = 2 | | | | | | | |
| IF4B_HUMAN | Eukaryotic translation initiation | SQS[166.9984]SDTEQQSPTSG | −1.98 | 0.1060 | 1.14 | 0.0260 | −1.74 | 0.2810 |
| | factor 4B OS = Homo sapiens | GGK | | | | | | |
| | GN = EIF4B PE = 1 SV = 2 | | | | | | | |
| IF4B_HUMAN | Eukaryotic translation initiation | TGS[166.9984]ESSQTGTSTTSSR | −1.11 | 0.0630 | 2.29 | 0.1450 | 2.06 | 0.5330 |
| | factor 4B OS = Homo sapiens | | | | | | | |
| | GN = EIF4B PE = 1 SV = 2 | | | | | | | |
| IFT52_HUMAN | Intraflagellar transport protein 52 | Y[243.0297]LDTGGDVFVMLG | −1.34 | 0.9040 | −1.53 | 0.2810 | −2.04 | 0.2830 |
| | homolog OS = Homo sapiens | EGGES[166.9984]R | | | | | | |
| | GN = IFT52 PE = 2 SV = 3 | | | | | | | |
| IL6RB_HUMAN | Interleukin-6 receptor subunit | SHIAQWS[166.9984]PHTPPR | 1.43 | 0.5680 | −2.23 | 0.0920 | −1.56 | 0.3440 |
| | beta OS = Homo sapiens GN = IL6ST | | | | | | | |
| | PE = 1 SV = 2 | | | | | | | |
| IPO4_HUMAN | Importin-4 OS = Homo sapiens | S[166.9984]FAVGTLAETIQGL | −1.13 | 0.7230 | −3.21 | 0.0002 | −3.64 | 0.0000 |
| | GN = IPO4 PE = 1 SV = 2 | GAAS[166.9984]AQFVSR | | | | | | |
| ITA5_HUMAN | Integrin alpha-5 OS = Homo sapiens | LLESSLS[166.9984]SSEGEEPV | 1.04 | 0.9200 | −1.43 | 0.3620 | −1.38 | 0.4070 |
| | GN = ITGA5 PE = 1 SV = 2 | EYK | | | | | | |
| ITAS_HUMAN | Integrin alpha-5 OS = Homo sapiens | LLESSLSS[166.9984]SEGEEPV | 1.25 | 0.6050 | −1.56 | 0.2700 | −1.24 | 0.6390 |
| | GN = ITGA5 PE = 1 SV = 2 | EYK | | | | | | |
| ITFG3_HUMAN | Protein ITFG3 OS = Homo sapiens | KS[166.9984]QENLGNPSKNE | 1.23 | 0.8780 | −1.17 | 0.8760 | 1.05 | 0.9820 |
| | GN = ITFG3 PE = 2 SV = 1 | DNVK | | | | | | |
| IWS1_HUMAN | Protein IWS1 homolog OS = Homo | AAVLS[166.9984]DS[166.9984] | 1.24 | 0.7200 | −1.23 | 0.6540 | 1.01 | 0.8840 |
| | sapiens GN = IWS1 PE = 1 SV = 2 | EDEEKASAK | | | | | | |
| IWS1_HUMAN | Protein IWS1 homolog OS = Homo | TIAS[166.9984]DS[166.9984] | −2.16 | 0.0360 | 1.76 | 0.2670 | −1.23 | 0.6750 |
| | sapiens GN = IWS1 PE = 1 SV = 2 | EEEAGKELSDK | | | | | | |
| JADE3_HUMAN | Protein Jade-3 OS = Homo sapiens | FAKS[166.9984]NGLEGSWSG | 1.41 | 0.6500 | −1.13 | 0.1000 | 1.25 | 0.0110 |
| | GN = PHF16 PE = 1 SV = 1 | NVTQK | | | | | | |
| JPH2_HUMAN | Junctophilin-2 OS = Homo sapiens | ETPRPEGGSPS[166.9984]PAG | 2.26 | 0.1950 | −1.42 | 0.6690 | 1.59 | 0.3300 |
| | GN = JPH2 PE = 1 SV = 2 | T[181.014]PPQPK | | | | | | |
| JPH2_HUMAN | Junctophilin-2 OS = Homo sapiens | GAGAAGLPQPPRES[166.9984] | 1.41 | 0.6500 | −1.02 | 0.9790 | 1.38 | 0.4600 |
| | GN = JPH2 PE = 1 SV = 2 | PQLHER | | | | | | |
| K0831_HUMAN | Uncharacterized protein KIAA0831 | M[147.0354]AS[166.9984]PS | 1.04 | 0.9980 | 1.06 | 0.9350 | 1.10 | 0.9170 |
| | OS = Homo sapiens GN = KIAA0831 | GKGAR | | | | | | |
| | PE = 1 SV = 1 | | | | | | | |
| K1143_HUMAN | Uncharacterized protein KIAA1143 | IQPQPPDEDGDHS[166.9984] | −1.13 | 0.8160 | −1.14 | 0.6540 | −1.28 | 0.4280 |
| | OS = Homo sapiens GN = KIAA1143 | DKEDEQPQVVVLK | | | | | | |
| | PE = 1 SV = 2 | | | | | | | |
| K1383_HUMAN | Uncharacterized protein KIAA1383 | SKAEC[160.0307]DNVGS[166.9984] | 1.13 | 0.8270 | 1.58 | 0.6240 | 1.80 | 0.4050 |
| | OS = Homo sapiens GN = KIAA1383 | VENGKTNSVVTC[160.0307] | | | | | | |
| | PE = 1 SV = 2 | S[166.9984]GAGNGR | | | | | | |
| K1704_HUMAN | Uncharacterized protein KIAA1704 | KQDDS[166.9984]PPRPIIGPAL | 1.40 | 0.4930 | −1.54 | 0.3160 | −1.09 | 0.9120 |
| | OS = Homo sapiens GN = KIAA1704 | PPGFIK | | | | | | |
| | PE = 1 SV = 1 | | | | | | | |
| K2C4_HUMAN | Keratin, type II cytoskeletal 4 - | RGAFSSVS[166.9984]M[147.0354] | 1.49 | 0.2230 | −1.67 | 0.3680 | −1.12 | 0.9500 |
| | Homo sapiens (Human) | SGGAGR | | | | | | |
| KAD1_HUMAN | Adenylate kinase isoenzyme 1 | KVNAEGS[166.9984]VDSVFS | 1.46 | 0.6710 | −1.61 | 0.5990 | −1.10 | 0.9040 |
| | OS = Homo sapiens GN = AK1 PE = 1 | QVC[160.0307]THLDALK | | | | | | |
| | SV = 3 | | | | | | | |
| KAD1_HUMAN | Adenylate kinase isoenzyme 1 | VNAEGS[166.9984]VDSVFSQ | −1.06 | 0.9370 | −1.01 | 0.9600 | −1.08 | 0.9620 |
| | OS = Homo sapiens GN = AK1 PE = 1 | VC[160.0307]THLDALK | | | | | | |
| | SV = 3 | | | | | | | |
| KAD1_HUMAN | Adenylate kinase isoenzyme 1 | YGYTHLS[166.9984]TGDLLR | 1.58 | 0.5740 | −2.00 | 0.3110 | −1.27 | 0.7430 |
| | OS = Homo sapiens GN = AK1 PE = 1 | | | | | | | |
| | SV = 3 | | | | | | | |
| KAP0_HUMAN | cAMP-dependent protein kinase | T[181.014]DSREDEISPPPPNP | 1.44 | 0.4490 | 1.13 | 0.9120 | 1.63 | 0.3320 |
| | type I-alpha regulatory subunit | VVK | | | | | | |
| | OS = Homo sapiens GN = PRKAR1A | | | | | | | |
| | PE = 1 SV = 1 | | | | | | | |
| KAP0_HUMAN | cAMP-dependent protein kinase | TDS[166.9984]REDEIS[166.9984] | −2.81 | 0.0040 | −1.77 | 0.3500 | −4.98 | 0.0000 |
| | type I-alpha regulatory subunit | PPPPNPVVK | | | | | | |
| | OS = Homo sapiens GN = PRKAR1A | | | | | | | |
| | PE = 1 SV = 1 | | | | | | | |
| KAP0_HUMAN | cAMP-dependent protein kinase | TDSREDEIS[166.9984]PPPPN | 1.28 | 0.8780 | 1.35 | 0.3500 | 1.73 | 0.0830 |
| | type I-alpha regulatory subunit | PVVK | | | | | | |
| | OS = Homo sapiens GN = PRKAR1A | | | | | | | |
| | PE = 1 SV = 1 | | | | | | | |
| KAP2_HUMAN | cAMP-dependent protein kinase | RVS[166.9984]VC[160.0307] | −1.04 | 0.9200 | −1.12 | 0.9750 | −1.15 | 0.9040 |
| | type II-alpha regulatory subunit | AETYNPDEEEEDTDPR | | | | | | |
| | OS = Homo sapiens GN = PRKAR2A | | | | | | | |
| | PE = 1 SV = 2 | | | | | | | |
| KAP2_HUMAN | cAMP-dependent protein kinase | VADAKGDS[166.9984]ES[166.9984] | 1.32 | 0.5720 | −1.19 | 0.7260 | 1.11 | 0.8560 |
| | type II-alpha regulatory subunit | EEDEDLEVPVPSR | | | | | | |
| | OS = Homo sapiens GN = PRKAR2A | | | | | | | |
| | PE = 1 SV = 2 | | | | | | | |
| KAP3_HUMAN | cAMP-dependent protein kinase | RAS[166.9984]VC[160.0307] | 1.39 | 0.7590 | 1.01 | 0.9940 | 1.40 | 0.7880 |
| | type II-beta regulatory subunit | AEAYNPDEEEDDAESR | | | | | | |
| | OS = Homo sapiens GN = PRKAR2B | | | | | | | |
| | PE = 1 SV = 3 | | | | | | | |
| KAPCA_HUMAN | cAMP-dependent protein kinase | TWT[181.014]LC[160.0307]G | −1.06 | 0.9480 | 1.03 | 0.9120 | −1.03 | 0.9800 |
| | catalytic subunit alpha OS = Homo | TPEYLAPEIILSK | | | | | | |
| | sapiens GN = PRKACA PE = 1 SV = 2 | | | | | | | |
| KAPCB_HUMAN | cAMP-dependent protein kinase | T[181.014]WTLC[160.0307]G | 1.34 | 0.9480 | −1.21 | 0.9600 | 1.11 | 0.8950 |
| | catalytic subunit beta OS = Homo | TPEYLAPEIILSK | | | | | | |
| | sapiens GN = PRKACB PE = 1 SV = 2 | | | | | | | |
| KCRM_HUMAN | Creatine kinase M-type OS = Homo | GQS[166.9984]IDDMIPAQK | −1.19 | 0.9370 | −2.11 | 0.3880 | −2.52 | 0.3830 |
| | sapiens GN = CKM PE = 1 SV = 2 | | | | | | | |
| KCRM_HUMAN | Creatine kinase M-type OS = Homo | GTGGVDTAAVGS[166.9984]V | −2.11 | 0.0370 | −1.79 | 0.5830 | −3.77 | 0.0001 |
| | sapiens GN = CKM PE = 1 SV = 2 | FDVSNADR | | | | | | |
| KCRM_HUMAN | Creatine kinase M-type OS = Homo | RGT[181.014]GGVDTAAVGSV | −1.24 | 0.8530 | −2.04 | 0.2720 | −2.52 | 0.0590 |
| | sapiens GN = CKM PE = 1 SV = 2 | FDVSNADR | | | | | | |
| KCRM_HUMAN | Creatine kinase M-type OS = Homo | RGTGGVDTAAVGS[166.9984] | −1.42 | 0.4620 | −3.67 | 0.0050 | −5.20 | 0.0000 |
| | sapiens GN = CKM PE = 1 SV = 2 | VFDVSNADR | | | | | | |
| KCRS_HUMAN | Creatine kinase, sarcomeric | LGYILTC[160.0307]PS[166.9984] | 1.30 | 0.9440 | −2.28 | 0.6810 | −1.76 | 0.4600 |
| | mitochondrial OS = Homo sapiens | NLGTGLR | | | | | | |
| | GN = CKMT2 PE = 1 SV = 2 | | | | | | | |
| KCRU_HUMAN | Creatine kinase, ubiquitous | LGYILTC[160.0307]PS[166.9984] | 1.02 | 0.9440 | −1.62 | 0.6810 | −1.60 | 0.4600 |
| | mitochondrial precursor - Homo | NLGTGLR | | | | | | |
| | sapiens (Human) | | | | | | | |
| KNG1_HUMAN | Kininogen-1 OS = Homo sapiens | ETTC[160.0307]S[166.9984]K | −1.30 | 0.1440 | 2.52 | 0.0010 | 1.93 | 0.0620 |
| | GN = KNG1 PE = 1 SV = 2 | ESNEELTESC[160.0307]ETK | | | | | | |
| KNG1_HUMAN | Kininogen-1 OS = Homo sapiens | ETTC[160.0307]S[166.9984]K | −1.05 | 0.9610 | 2.47 | 0.0530 | 2.36 | 0.1370 |
| | GN = KNG1 PE = 1 SV = 2 | ESNEELTESC[160.0307]ETKK | | | | | | |
| KNG1_HUMAN | Kininogen-1 OS = Homo sapiens | ETTC[160.0307]SKES[166.9984] | −1.39 | 0.6260 | 4.07 | 0.0000 | 2.93 | 0.0240 |
| | GN = KNG1 PE = 1 SV = 2 | NEELTESC[160.0307]ETK | | | | | | |
| KNG1_HUMAN | Kininogen-1 OS = Homo sapiens | ETTC[160.0307]SKES[166.9984] | −1.10 | 0.7000 | 2.16 | 0.0080 | 1.96 | 0.0540 |
| | GN = KNG1 PE = 1 SV = 2 | NEELTESC[160.0307]ETKK | | | | | | |
| KPCG_HUMAN | Protein kinase C gamma type | LHISDFSFLM[147.0354]VLGK | −1.10 | 0.7650 | −1.07 | 0.9410 | −1.18 | 0.6420 |
| | OS = Homo sapiens GN = PRKCG | GS[166.9984]FGK | | | | | | |
| | PE = 1 SV = 3 | | | | | | | |
| LA_HUMAN | Lupus La protein OS = Homo | FAS[166.9984]DDEHDEHDEN | 1.14 | 0.7850 | −1.19 | 0.6760 | −1.05 | 0.9190 |
| | sapiens GN = SSB PE = 1 SV = 2 | GATGPVK | | | | | | |
| LA_HUMAN | Lupus La protein OS = Homo | FAS[166.9984]DDEHDEHDEN | 1.35 | 0.7100 | −1.45 | 0.5850 | −1.07 | 0.7650 |
| | sapiens GN = SSB PE = 1 SV = 2 | GATGPVKR | | | | | | |
| LAP2A_HUMAN | Lamina-associated polypeptide 2 | GPPDFS[166.9984]S[166.9984] | 2.19 | 0.0610 | −1.87 | 0.2240 | 1.17 | 0.8160 |
| | isoform alpha - Homo sapiens | DEEREPTPVLGSGAAAAGR | | | | | | |
| | (Human) | | | | | | | |
| LAP2A_HUMAN | Lamina-associated polypeptide 2 | GPPDFSSDEEREPT[181.014]P | 1.76 | 0.1070 | −1.13 | 0.8320 | 1.55 | 0.2430 |
| | isoform alpha - Homo sapiens | VLGSGAAAAGR | | | | | | |
| | (Human) | | | | | | | |
| LAP2A_HUMAN | Lamina-associated polypeptide 2 | SST[181.014]PLPTISSSAENTR | 2.04 | 0.0006 | −1.58 | 0.0720 | 1.30 | 0.5540 |
| | isoform alpha - Homo sapiens | | | | | | | |
| | (Human) | | | | | | | |
| LARP7_HUMAN | La-related protein 7 OS = Homo | KRSS[166.9984]S[166.9984]E | −4.46 | 0.0100 | 3.48 | 0.0070 | −1.28 | 0.8900 |
| | sapiens GN = LARP7 PE = 1 SV = 1 | DAESLAPR | | | | | | |
| LARP7_HUMAN | La-related protein 7 OS = Homo | S[166.9984]RPTS[166.9984]E | 1.19 | 0.8830 | −1.42 | 0.6460 | −1.19 | 0.8130 |
| | sapiens GN = LARP7 PE = 1 SV = 1 | GSDIESTEPQK | | | | | | |
| LARP7_HUMAN | La-related protein 7 OS = Homo | SRPT[181.014]S[166.9984]EG | 1.09 | 0.9460 | −1.28 | 0.7660 | −1.18 | 0.7890 |
| | sapiens GN = LARP7 PE = 1 SV = 1 | SDIESTEPQK | | | | | | |
| LARP7_HUMAN | La-related protein 7 OS = Homo | SRPT[181.014]SEGSDIESTEPQK | 1.18 | 0.8590 | −1.03 | 0.9410 | 1.14 | 0.9030 |
| | sapiens GN = LARP7 PE = 1 SV = 1 | | | | | | | |
| LASP1_HUMAN | LIM and SH3 domain protein 1 - | MGPSGGEGM[147.0354]EPE | 1.59 | 0.6820 | 1.02 | 0.9980 | 1.61 | 0.7030 |
| | Homo sapiens (Human) | RRDSQDGS[166.9984]SYR | | | | | | |
| LASP1_HUMAN | LIM and SH3 domain protein 1 - | MGPSGGEGMEPERRDS[166.9984] | −2.12 | 0.7070 | 2.85 | 0.5150 | 1.34 | 0.8950 |
| | Homo sapiens (Human) | QDGSSYR | | | | | | |
| LBH_HUMAN | Protein LBH OS = Homo sapiens | LPS[166.9984]IVVEPTEGEVES | −1.65 | 0.5430 | 1.18 | 0.9100 | −1.40 | 0.7470 |
| | GN = LBH PE = 1 SV = 1 | GELR | | | | | | |
| LEO1_HUMAN | RNA polymerase-associated | KLT[181.014]SDEEGEPSGK | 1.14 | 0.9200 | −1.34 | 0.7270 | −1.17 | 0.7650 |
| | protein LEO1 OS = Homo sapiens | | | | | | | |
| | GN = LEO1 PE = 1 SV = 1 | | | | | | | |
| LETM2_HUMAN | LETM1 domain-containing protein | QVQTGHKPS[166.9984]TKEIVR | 1.63 | 0.7850 | 2.31 | 0.3860 | 3.76 | 0.1030 |
| | LETM2, mitochondrial OS = Homo | | | | | | | |
| | sapiens GN = LETM2 PE = 2 SV = 2 | | | | | | | |
| LIGO2_HUMAN | Leucine-rich repeat and | ATVLGDGTLEIRFAQDQDSGM | −1.48 | 0.6710 | −1.62 | 0.3790 | −2.39 | 0.2170 |
| | immunoglobulin-like domain- | [147.0354]YVC[160.0307]IAS | | | | | | |
| | containing nogo receptor- | NAAGNDT[181.014]FT[181.014] | | | | | | |
| | interacting protein 2 OS = Homo | AS[166.9984]LT[181.014] | | | | | | |
| | sapiens GN = LINGO2 PE = 2 SV = 1 | VKGFAS[166.9984]DR | | | | | | |
| LIMA1_HUMAN | LIM domain and actin-binding | ETPHS[166.9984]PGVEDAPIAK | 1.98 | 0.4490 | 1.44 | 0.6930 | 2.85 | 0.1010 |
| | protein 1 - Homo sapiens (Human) | | | | | | | |
| LIMC1_HUMAN | LIM and calponin homology | GSSDGRGS[166.9984]DSESDL | 1.48 | 0.6360 | −1.07 | 0.9220 | 1.38 | 0.8250 |
| | domains-containing protein 1 | PHR | | | | | | |
| | OS = Homo sapiens GN = LIMCH1 | | | | | | | |
| | PE = 1 SV = 3 | | | | | | | |
| LIMC1_HUMAN | LIM and calponin homology | S[166.9984]PEPEATLTFPFLDK | 1.05 | 0.9460 | 1.46 | 0.8180 | 1.54 | 0.6750 |
| | domains-containing protein 1 | | | | | | | |
| | OS = Homo sapiens GN = LIMCH1 | | | | | | | |
| | PE = 1 SV = 3 | | | | | | | |
| LMNA_HUMAN | Lamin-A/C - Homo sapiens | LRLS[166.9984]PS[166.9984] | 2.54 | 0.0770 | −1.69 | 0.4010 | 1.50 | 0.6150 |
| | (Human) | PTSQR | | | | | | |
| LMNA_HUMAN | Lamin-A/C - Homo sapiens | SGAQASSTPLS[166.9984]PTR | 1.50 | 0.6980 | −1.20 | 0.8590 | 1.25 | 0.8730 |
| | (Human) | | | | | | | |
| LMO7_HUMAN | LIM domain only protein 7 | RGES[166.9984]LDNLDSPR | 2.79 | 0.0340 | 1.32 | 0.7820 | 3.67 | 0.0040 |
| | OS = Homo sapiens GN = LMO7 PE = 1 | | | | | | | |
| | SV = 2 | | | | | | | |
| LMO7_HUMAN | LIM domain only protein 7 | SAS[166.9984]VNKEPVSLPGI | 2.73 | 0.7490 | −1.68 | 0.9980 | 1.63 | 0.5650 |
| | OS = Homo sapiens GN = LMO7 PE = 1 | M[147.0354]R | | | | | | |
| | SV = 2 | | | | | | | |
| LMO7_HUMAN | LIM domain only protein 7 | SAS[166.9984]VNKEPVSLPGI | −1.11 | 0.9060 | −1.27 | 0.6460 | −1.41 | 0.8250 |
| | OS = Homo sapiens GN = LMO7 PE = 1 | MR | | | | | | |
| | SV = 2 | | | | | | | |
| LMO7_HUMAN | LIM domain only protein 7 | SHS[166.9984]PSASQSGSQLR | 2.50 | 0.2110 | 1.08 | 0.9080 | 2.71 | 0.1470 |
| | OS = Homo sapiens GN = LMO7 PE = 1 | | | | | | | |
| | SV = 2 | | | | | | | |
| LMOD1_HUMAN | Leiomodin-1 OS = Homo sapiens | GS[166.9984]PKPS[166.9984] | 4.40 | 0.0400 | −6.84 | 0.0020 | −1.56 | 0.7110 |
| | GN = LMOD1 PE = 1 SV = 2 | PQPSPKPS[166.9984]PK | | | | | | |
| LMOD1_HUMAN | Leiomodin-1 OS = Homo sapiens | GSPKPSPQPS[166.9984]PKPS | 9.66 | 0.0000 | −1.92 | 0.0001 | 5.04 | 0.0080 |
| | GN = LMOD1 PE = 1 SV = 2 | PK | | | | | | |
| LMOD1_HUMAN | Leiomodin-1 OS = Homo sapiens | NSLS[166.9984]PATQR | 9.18 | 0.0000 | −4.93 | 0.0030 | 1.86 | 0.7820 |
| | GN = LMOD1 PE = 1 SV = 2 | | | | | | | |
| LMOD2_HUMAN | Leiomodin-2 OS = Homo sapiens | GTPSSSPYVS[166.9984]PR | −1.88 | 0.6030 | 2.34 | 0.6680 | 1.24 | 0.9900 |
| | GN = LMOD2 PE = 2 SV = 2 | | | | | | | |
| LMOD2_HUMAN | Leiomodin-2 OS = Homo sapiens | S[166.9984]RPLS[166.9984]P | 1.59 | 0.4590 | −1.39 | 0.6810 | 1.14 | 0.9380 |
| | GN = LMOD2 PE = 2 SV = 2 | VATPPPPPPPPPPPPPSSQR | | | | | | |
| LMOD2_HUMAN | Leiomodin-2 OS = Homo sapiens | S[166.9984]RPLSPVATPPPPPP | 1.25 | 0.7070 | −1.25 | 0.6950 | 1.00 | 0.9960 |
| | GN = LMOD2 PE = 2 SV = 2 | PPPPPPPSSQR | | | | | | |
| LMOD2_HUMAN | Leiomodin-2 OS = Homo sapiens | SRPLS[166.9984]PVAT[181.014] | 1.41 | 0.6120 | −1.21 | 0.8060 | 1.16 | 0.9120 |
| | GN = LMOD2 PE = 2 SV = 2 | PPPPPPPPPPPPPSSQR | | | | | | |
| LMOD2_HUMAN | Leiomodin-2 OS = Homo sapiens | SRPLS[166.9984]PVATPPPPPP | 1.28 | 0.6610 | −1.15 | 0.7970 | 1.11 | 0.8840 |
| | GN = LMOD2 PE = 2 SV = 2 | PPPPPPPSSQR | | | | | | |
| LMOD2_HUMAN | Leiomodin-2 OS = Homo sapiens | YES[166.9984]IDEDELLASLSA | 1.66 | 0.4530 | −1.57 | 0.6040 | 1.06 | 0.9100 |
| | GN = LMOD2 PE = 2 SV = 2 | EELKELER | | | | | | |
| LRC57_HUMAN | Leucine-rich repeat-containing | DRGLT[181.014]EFPADLQKLT | −5.13 | 0.7000 | 1.93 | 0.9830 | −2.66 | 0.7160 |
| | protein 57 OS = Homo sapiens | [181.014]S[166.9984]NLR | | | | | | |
| | GN = LRRC57 PE = 1 SV = 1 | | | | | | | |
| MAP1B_HUMAN | Microtubule-associated protein 1B | KLGDVS[166.9984]PTQIDVSQ | 1.99 | 0.4720 | −1.22 | 0.9340 | 1.64 | 0.5520 |
| | OS = Homo sapiens GN = MAP1B | FGSFK | | | | | | |
| | PE = 1 SV = 1 | | | | | | | |
| MAP1B_HUMAN | Microtubule-associated protein 1B | SPSLSPSPPS[166.9984]PLEK | 2.00 | 0.4690 | 1.14 | 0.9190 | 2.29 | 0.2410 |
| | OS = Homo sapiens GN = MAP1B | | | | | | | |
| | PE = 1 SV = 1 | | | | | | | |
| MAP4_HUMAN | Microtubule-associated protein 4 - | DM[147.0354]ES[166.9984]P | 4.53 | 0.2460 | −1.30 | 0.9400 | 3.49 | 0.0540 |
| | Homo sapiens (Human) | TKLDVTLAK | | | | | | |
| MAP4_HUMAN | Microtubule-associated protein 4 - | SKVGS[166.9984]TENIK | 3.80 | 0.1450 | 1.50 | 0.9660 | 5.68 | 0.0640 |
| | Homo sapiens (Human) | | | | | | | |
| MAP4_HUMAN | Microtubule-associated protein 4 - | VGS[166.9984]LDNVGHLPAG | 3.32 | 0.0190 | 1.37 | 0.5590 | 4.55 | 0.0000 |
| | Homo sapiens (Human) | GAVK | | | | | | |
| MARCS_HUMAN | Myristoylated alanine-rich C- | AEDGATPSPSNET[181.014]PK | 1.84 | 0.4490 | 1.75 | 0.2360 | 3.23 | 0.0020 |
| | kinase substrate - Homo sapiens | | | | | | | |
| | (Human) | | | | | | | |
| MARCS_HUMAN | Myristoylated alanine-rich C- | AEDGATPSPSNET[181.014]PKK | 1.61 | 0.0000 | 1.62 | 0.0290 | 2.61 | 0.1760 |
| | kinase substrate - Homo sapiens | | | | | | | |
| | (Human) | | | | | | | |
| MARCS_HUMAN | Myristoylated alanine-rich C- | EAPAEGEAAEPGS[166.9984]P | −1.99 | 0.3080 | 1.39 | 0.6260 | −1.43 | 0.7510 |
| | kinase substrate - Homo sapiens | TAAEGEAASAASSTSSPK | | | | | | |
| | (Human) | | | | | | | |
| MARCS_HUMAN | Myristoylated alanine-rich C- | GEPAAAAAPEAGAS[166.9984] | −2.16 | 0.4490 | 2.66 | 0.0610 | 1.23 | 0.7710 |
| | kinase substrate - Homo sapiens | PVEK | | | | | | |
| | (Human) | | | | | | | |
| MARCS_HUMAN | Myristoylated alanine-rich C- | LSGFS[166.9984]FK | 2.15 | 0.3190 | −1.00 | 0.8580 | 2.15 | 0.0440 |
| | kinase substrate - Homo sapiens | | | | | | | |
| | (Human) | | | | | | | |
| MARCS_HUMAN | Myristoylated alanine-rich C- | LSGFS[166.9984]FKK | 1.89 | 0.2410 | −1.27 | 0.7430 | 1.50 | 0.2940 |
| | kinase substrate - Homo sapiens | | | | | | | |
| | (Human) | | | | | | | |
| MARE2_HUMAN | Microtubule-associated protein | SSPAAKPGSTPS[166.9984]RP | 1.48 | 0.8360 | −2.20 | 0.4850 | −1.48 | 0.6640 |
| | RP/EB family member 2 OS = Homo | S[166.9984]SAKR | | | | | | |
| | sapiens GN = MAPRE2 PE = 1 SV = 1 | | | | | | | |
| MATR3_HUMAN | Matrin-3 - Homo sapiens (Human) | RDS[166.9984]FDDRGPSLNP | −2.99 | 0.0780 | 1.78 | 0.5750 | −1.68 | 0.6060 |
| | | VLDYDHGSR | | | | | | |
| MATR3_HUMAN | Matrin-3 - Homo sapiens (Human) | SYS[166.9984]PDGKESPSDKK | 2.61 | 0.0060 | −1.11 | 0.0003 | 2.36 | 0.0750 |
| MECP2_HUMAN | Methyl-CpG-binding protein 2 | AETSEGSGSAPAVPEASAS[166.9984] | 1.72 | 0.2110 | −1.64 | 0.3480 | 1.05 | 0.8900 |
| | OS = Homo sapiens GN = MECP2 | PK | | | | | | |
| | PE = 1 SV = 1 | | | | | | | |
| MEGF8_HUMAN | Multiple epidermal growth factor- | S[166.9984]FHAAAYVPAGRG | −1.61 | 0.7070 | −1.13 | 0.7140 | −1.81 | 0.4960 |
| | like domains 8 OS = Homo sapiens | AM[147.0354]YLLGGLT[181.014] | | | | | | |
| | GN = MEGF8 PE = 1 SV = 1 | AGGVT[181.014]RDFWVL | | | | | | |
| | | NLTTLQWR | | | | | | |
| MICA3_HUMAN | Protein MICAL-3 OS = Homo sapiens | Y[243.0297]M[147.0354]ATQ | −1.17 | 0.9580 | 1.74 | 0.7800 | 1.48 | 0.8520 |
| | GN = MICAL3 PE = 1 SV = 1 | LLAK | | | | | | |
| MLRV_HUMAN | Myosin regulatory light chain 2, | AGGANS[166.9984]NVFSM[147.0354] | 2.86 | 0.3420 | −1.86 | 0.7450 | 1.53 | 0.4090 |
| | ventricular/cardiac muscle isoform | FEQTQIQEFK | | | | | | |
| | OS = Homo sapiens GN = MYL2 PE = 1 | | | | | | | |
| | SV = 3 | | | | | | | |
| MLRV_HUMAN | Myosin regulatory light chain 2, | AGGANS[166.9984]NVFSMFE | −3.53 | 0.0060 | 1.70 | 0.7800 | −2.08 | 0.0880 |
| | ventricular/cardiac muscle isoform | QTQIQEFK | | | | | | |
| | OS = Homo sapiens GN = MYL2 PE = 1 | | | | | | | |
| | SV = 3 | | | | | | | |
| MLRV_HUMAN | Myosin regulatory light chain 2, | RAGGANS[166.9984]NVFSM | 3.01 | 0.2020 | −1.98 | 0.5540 | 1.52 | 0.7030 |
| | ventricular/cardiac muscle isoform | [147.0354]FEQTQIQEFK | | | | | | |
| | OS = Homo sapiens GN = MYL2 PE = 1 | | | | | | | |
| | SV = 3 | | | | | | | |
| MLRV_HUMAN | Myosin regulatory light chain 2, | RAGGANS[166.9984]NVFSMF | −2.29 | 0.3480 | 1.88 | 0.5550 | −1.22 | 0.8800 |
| | ventricular/cardiac muscle isoform | EQTQIQEFK | | | | | | |
| | OS = Homo sapiens GN = MYL2 PE = 1 | | | | | | | |
| | SV = 3 | | | | | | | |
| MPRI_HUMAN | Cation-independent mannose-6- | ALSS[166.9984]LHGDDQDSE | 1.08 | 0.9040 | −1.71 | 0.1900 | −1.58 | 0.2460 |
| | phosphate receptor OS = Homo | DEVLTIPEVK | | | | | | |
| | sapiens GN = IGF2R PE = 1 SV = 2 | | | | | | | |
| MPRI_HUMAN | Cation-independent mannose-6- | ALSSLHGDDQDS[166.9984]E | −1.02 | 0.9840 | −1.34 | 0.6460 | −1.36 | 0.5390 |
| | phosphate receptor OS = Homo | DEVLTIPEVK | | | | | | |
| | sapiens GN = IGF2R PE = 1 SV = 2 | | | | | | | |
| MPRI_HUMAN | Cation-independent mannose-6- | LVS[166.9984]FHDDS[166.9984] | −2.64 | 0.0120 | 1.03 | 0.9350 | −2.58 | 0.0360 |
| | phosphate receptor OS = Homo | DEDLLHI | | | | | | |
| | sapiens GN = IGF2R PE = 1 SV = 2 | | | | | | | |
| MPRI_HUMAN | Cation-independent mannose-6- | LVSFHDDS[166.9984]DEDLLHI | 1.30 | 0.5270 | −2.01 | 0.0100 | −1.55 | 0.2930 |
| | phosphate receptor OS = Homo | | | | | | | |
| | sapiens GN = IGF2R PE = 1 SV = 2 | | | | | | | |
| MPRI_HUMAN | Cation-independent mannose-6- | S[166.9984]LQLSTEGFITLTY[243.0297]K | 1.18 | 0.8240 | −1.04 | 0.9670 | 1.14 | 0.8080 |
| | phosphate receptor OS = Homo | | | | | | | |
| | sapiens GN = IGF2R PE = 1 SV = 2 | | | | | | | |
| MRLC2_HUMAN | Myosin regulatory light chain | AT[181.014]SNVFAM[147.0354] | 3.22 | 0.3500 | −6.30 | 0.0390 | −1.96 | 0.1300 |
| | MRLC2 OS = Homo sapiens | FDQSQIQEFK | | | | | | |
| | GN = MYLC2B PE = 1 SV = 2 | | | | | | | |
| MRLC2_HUMAN | Myosin regulatory light chain | AT[181.014]SNVFAMFDQSQI | −2.19 | 0.5860 | 1.04 | 0.7820 | −2.11 | 0.4980 |
| | MRLC2 OS = Homo sapiens | QEFK | | | | | | |
| | GN = MYLC2B PE = 1 SV = 2 | | | | | | | |
| MRLC2_HUMAN | Myosin regulatory light chain | ATS[166.9984]NVFAM[147.0354] | 4.47 | 0.4170 | −6.46 | 0.1900 | −1.45 | 0.5810 |
| | MRLC2 OS = Homo sapiens | FDQSQIQEFK | | | | | | |
| | GN = MYLC2B PE = 1 SV = 2 | | | | | | | |
| MTA2 _HUMAN | Metastasis-associated protein | RPY[243.0297]APINANAIKAE | −1.01 | 0.9480 | −1.44 | 0.6810 | −1.45 | 0.8240 |
| | MTA2 OS = Homo sapiens | C[160.0307]SIR | | | | | | |
| | GN = MTA2 PE = 1 SV = 1 | | | | | | | |
| MUCSB_HUMAN | Mucin-5B OS = Homo sapiens | ATSSSSPRT[181.014]AT[181.014] | 1.57 | 0.8390 | 3.31 | 0.0700 | 5.21 | 0.0040 |
| | GN = MUC5B PE = 1 SV = 2 | T[181.014]LPVLT[181.014] | | | | | | |
| | | STATK | | | | | | |
| MYCT1_HUMAN | Myc target protein 1 OS = Homo | SS[166.9984]YTHGLNR | 1.19 | 0.7000 | 2.49 | 0.2100 | 2.95 | 0.0660 |
| | sapiens GN = MYCT1 PE = 2 SV = 1 | | | | | | | |
| MYH14_HUMAN | Myosin-14 - Homo sapiens | QLVS[166.9984]TLEK | 1.07 | 0.9870 | 2.24 | 0.5810 | 2.40 | 0.5180 |
| | (Human) | | | | | | | |
| MYH15_HUMAN | Myosin-15 OS = Homo sapiens | EKRT[181.014]TEHK | −1.02 | 0.9190 | −3.25 | 0.0560 | −3.33 | 0.2030 |
| | GN = MYH15 PE = 1 SV = 3 | | | | | | | |
| MYOZ2_HUMAN | Myozenin-2 OS = Homo sapiens | DIMLEELSHLS[166.9984]NR | −1.26 | 0.9500 | 1.55 | 0.1770 | 1.23 | 0.4280 |
| | GN = MYOZ2 PE = 1 SV = 1 | | | | | | | |
| MYOZ2_HUMAN | Myozenin-2 OS = Homo sapiens | S[166.9984]PPNPDNIAPGYSG | 1.20 | 0.5420 | −1.48 | 0.2420 | −1.23 | 0.4520 |
| | GN = MYOZ2 PE = 1 SV = 1 | PLK | | | | | | |
| MYOZ2_HUMAN | Myozenin-2 OS = Homo sapiens | VDGSNLEGGS[166.9984]QQA | 1.60 | 0.8500 | −1.22 | 0.9980 | 1.31 | 0.8240 |
| | GN = MYOZ2 PE = 1 SV = 1 | PLTPPNTPDPR | | | | | | |
| MYOZ2_HUMAN | Myozenin-2 OS = Homo sapiens | VDGSNLEGGSQQAPLT[181.014] | −1.53 | 0.1820 | 3.02 | 0.0003 | 1.97 | 0.0030 |
| | GN = MYOZ2 PE = 1 SV = 1 | PPNT[181.014]PDPR | | | | | | |
| MYOZ2_HUMAN | Myozenin-2 OS = Homo sapiens | VDGSNLEGGSQQAPLT[181.014] | −1.03 | 0.9810 | −1.24 | 0.4450 | −1.28 | 0.2190 |
| | GN = MYOZ2 PE = 1 SV = 1 | PPNTPDPR | | | | | | |
| MYPC3_HUMAN | Myosin-binding protein C, cardiac- | IS[166.9984]DSHEDTGILDFSS | −1.07 | 0.9350 | −1.10 | 0.9920 | −1.18 | 0.9060 |
| | type OS = Homo sapiens | LLK | | | | | | |
| | GN = MYBPC3 PE = 1 SV = 3 | | | | | | | |
| MYPC3_HUMAN | Myosin-binding protein C, cardiac- | RIS[166.9984]DSHEDTGILDFS | 1.26 | 0.9200 | −1.26 | 0.8180 | 1.00 | 0.8160 |
| | type OS = Homo sapiens | SLLK | | | | | | |
| | GN = MYBPC3 PE = 1 SV = 3 | | | | | | | |
| MYPT1_HUMAN | Protein phosphatase 1 regulatory | KTGS[166.9984]YGALAEITASK | 1.21 | 0.7790 | 1.21 | 0.9060 | 1.46 | 0.6730 |
| | subunit 12A OS = Homo sapiens | | | | | | | |
| | GN = PPP1R12A PE = 1 SV = 1 | | | | | | | |
| MYPT1_HUMAN | Protein phosphatase 1 regulatory | KTGSY[243.0297]GALAEITASK | 1.41 | 0.7030 | 1.31 | 0.6540 | 1.84 | 0.1880 |
| | subunit 12A OS = Homo sapiens | | | | | | | |
| | GN = PPP1R12A PE = 1 SV = 1 | | | | | | | |
| MYPT1_HUMAN | Protein phosphatase 1 regulatory | RS[166.9984]TQGVTLTDLQEA | −2.68 | 0.0260 | 1.25 | 0.6460 | −2.15 | 0.1140 |
| | subunit 12A OS = Homo sapiens | EK | | | | | | |
| | GN = PPP1R12A PE = 1 SV = 1 | | | | | | | |
| MYPT1_HUMAN | Protein phosphatase 1 regulatory | RST[181.014]QGVTLTDLQEAEK | −1.92 | 0.3180 | 1.20 | 0.8230 | −1.60 | 0.4280 |
| | subunit 12A OS = Homo sapiens | | | | | | | |
| | GN = PPP1R12A PE = 1 SV = 1 | | | | | | | |
| MYPT1_HUMAN | Protein phosphatase 1 regulatory | S[166.9984]YLTPVRDEESESQR | −1.02 | 0.8880 | 1.28 | 0.7520 | 1.25 | 0.8130 |
| | subunit 12A OS = Homo sapiens | | | | | | | |
| | GN = PPP1R12A PE = 1 SV = 1 | | | | | | | |
| MYPT2_HUMAN | Protein phosphatase 1 regulatory | DEDET[181.014]DGSEEVKET | 2.24 | 0.5910 | −3.41 | 0.1660 | −1.52 | 0.3420 |
| | subunit 12B OS = Homo sapiens | WHER | | | | | | |
| | GN = PPP1R12B PE = 1 SV = 2 | | | | | | | |
| MYPT2_HUMAN | Protein phosphatase 1 regulatory | S[166.9984]LDEEPIC[160.0307] | −2.20 | 0.1120 | −1.37 | 0.6460 | −3.03 | 0.0040 |
| | subunit 12B OS = Homo sapiens | HR | | | | | | |
| | GN = PPP1R12B PE = 1 SV = 2 | | | | | | | |
| NCAM1_HUMAN | Neural cell adhesion molecule 1 | AAFSKDES[166.9984]KEPIVEVR | −1.09 | 0.9040 | −1.66 | 0.5830 | −1.81 | 0.3900 |
| | OS = Homo sapiens GN = NCAM1 | | | | | | | |
| | PE = 1 SV = 3 | | | | | | | |
| NCK1_HUMAN | Cytoplasmic protein NCK1 | RKPS[166.9984]VPDSASPADD | −1.20 | 0.8500 | 1.22 | 0.8220 | 1.01 | 0.9830 |
| | OS = Homo sapiens GN = NCK1 PE = 1 | SFVDPGER | | | | | | |
| | SV = 1 | | | | | | | |
| NDK8_HUMAN | Putative nucleoside diphosphate | VMLGET[181.014]NPADSKPG | −1.66 | 0.6300 | 1.23 | 0.9210 | −1.36 | 0.8240 |
| | kinase - Homo sapiens (Human) | TIR | | | | | | |
| NDRG1_HUMAN | Protein NDRG1 OS = Homo sapiens | SHT[181.014]SEGAHLDITPNS | 1.39 | 0.7050 | 1.11 | 0.9160 | 1.54 | 0.5450 |
| | GN = NDRG1 PE = 1 SV = 1 | GAAGNSAGPK | | | | | | |
| NDRG3_HUMAN | Protein NDRG3 OS = Homo sapiens | C[160.0307]S[166.9984]TLLV | 1.53 | 0.7260 | −1.34 | 0.8520 | 1.15 | 0.8770 |
| | GN = NDRG3 PE = 1 SV = 2 | VGDNSPAVEAVVEC[160.0307] | | | | | | |
| | | NS[166.9984]R | | | | | | |
| NEBL_HUMAN | Nebulette OS = Homo sapiens | TDPGS[166.9984]IFDLDPLED | 1.58 | 0.7070 | 1.75 | 0.6460 | 2.78 | 0.1760 |
| | GN = NEBL PE = 1 SV = 1 | NIQSR | | | | | | |
| NEUG_HUMAN | Neurogranin OS = Homo sapiens | IQAS[166.9984]FR | 1.27 | 0.8660 | 1.20 | 0.5070 | 1.52 | 0.6760 |
| | GN = NRGN PE = 1 SV = 1 | | | | | | | |
| NEXN_HUMAN | Nexilin OS = Homo sapiens | EM[147.0354]LAS[166.9984] | 5.55 | 0.1450 | −5.85 | 0.1760 | −1.05 | 0.7240 |
| | GN = NEXN PE = 1 SV = 1 | DDEEDVSSK | | | | | | |
| NEXN_HUMAN | Nexilin OS = Homo sapiens | EM[147.0354]LAS[166.9984] | 13.46 | 0.0005 | −16.05 | 0.0003 | −1.19 | 0.6860 |
| | GN = NEXN PE = 1 SV = 1 | DDEEDVSSKVEK | | | | | | |
| NEXN_HUMAN | Nexilin OS = Homo sapiens | TIS[166.9984]QEFLTPGK | 2.79 | 0.1370 | −1.15 | 0.9240 | 2.42 | 0.0020 |
| | GN = NEXN PE = 1 SV = 1 | | | | | | | |
| NFAC4_HUMAN | Nuclear factor of activated T-cells, | DPGS[166.9984]PGPFDYVGA | 1.50 | 0.6600 | −1.41 | 0.7210 | 1.06 | 0.9720 |
| | cytoplasmic 4 OS = Homo sapiens | PPAES[166.9984]IPQKT[181.014] | | | | | | |
| | GN = NFATC4 PE = 1 SV = 2 | RR | | | | | | |
| NFM_HUMAN | Neurofilament medium | GKS[166.9984]PVPKS[166.9984] | −1.61 | 0.4550 | 1.22 | 0.5750 | −1.31 | 0.9040 |
| | polypeptide OS = Homo sapiens | PVEEK | | | | | | |
| | GN = NEFM PE = 1 SV = 2 | | | | | | | |
| NFYC_HUMAN | Nuclear transcription factor Y | S[166.9984]T[181.014]EGGF | −2.73 | 0.0940 | 1.45 | 0.7920 | −1.88 | 0.4850 |
| | subunit gamma OS = Homo sapiens | GGT[181.014]SSSDAQQSLQS | | | | | | |
| | GN = NFYC PE = 1 SV = 3 | [166.9984]FWPRVM[147.0354] | | | | | | |
| | | EEIR | | | | | | |
| NOC3L_HUMAN | Nucleolar complex protein 3 | LKLHT[181.014]ET[181.014]L | −1.73 | 0.1810 | −1.03 | 0.7430 | −1.78 | 0.1280 |
| | homolog OS = Homo sapiens | NIVFVT[181.014]YFR | | | | | | |
| | GN = NOC3L PE = 1 SV = 1 | | | | | | | |
| NOLC1_HUMAN | Nucleolar phosphoprotein p130 | GKGS[166.9984]PRPQAPK | 1.70 | 0.8540 | −4.87 | 0.1300 | −2.86 | 0.4280 |
| | OS = Homo sapiens GN = NOLC1 | | | | | | | |
| | PE = 1 SV = 2 | | | | | | | |
| NOS1_HUMAN | Nitric oxide synthase, brain | NTALGVIS[166.9984]NWTDELR | 1.35 | 0.9040 | 1.87 | 0.3500 | 2.52 | 0.1200 |
| | OS = Homo sapiens GN = NOS1 PE = 1 | | | | | | | |
| | SV = 2 | | | | | | | |
| NP1L4_HUMAN | Nucleosome assembly protein 1- | EFITGDVEPTDAESEWHS[166.9984] | −1.12 | 0.5680 | 1.44 | 0.3880 | 1.28 | 0.5850 |
| | like 4 OS = Homo sapiens | ENEEEEK | | | | | | |
| | GN = NAP1L4 PE = 1 SV = 1 | | | | | | | |
| NP1L4_HUMAN | Nucleosome assembly protein 1- | EFITGDVEPTDAESEWHS[166.9984] | −1.46 | 0.8780 | 1.36 | 0.7670 | −1.07 | 0.9560 |
| | like 4 OS = Homo sapiens | ENEEEEKLAGDMK | | | | | | |
| | GN = NAP1L4 PE = 1 SV = 1 | | | | | | | |
| NSF1C_HUMAN | NSFL1 cofactor p47 OS = Homo | KKS[166.9984]PNELVDDLFK | 1.36 | 0.8490 | 1.03 | 0.8710 | 1.39 | 0.4790 |
| | sapiens GN = NSFL1C PE = 1 SV = 2 | | | | | | | |
| NUCB1_HUMAN | Nucleobindin-1 OS = Homo sapiens | AQRLS[166.9984]QETEALGR | 1.78 | 0.2110 | −1.60 | 0.4340 | 1.11 | 0.9020 |
| | GN = NUCB1 PE = 1 SV = 4 | | | | | | | |
| NUCKS_HUMAN | Nuclear ubiquitous casein and | KVVDYSQFQES[166.9984]DD | −1.07 | 0.9060 | −1.33 | 0.6400 | −1.42 | 0.3250 |
| | cyclin-dependent kinases substrate | ADEDYGR | | | | | | |
| | OS = Homo sapiens GN = NUCKS1 | | | | | | | |
| | PE = 1 SV = 1 | | | | | | | |
| NUCKS_HUMAN | Nuclear ubiquitous casein and | TPSPKEEDEEPES[166.9984]PP | −1.33 | 0.2270 | −1.41 | 0.7800 | −1.89 | 0.0110 |
| | cyclin-dependent kinases substrate | EKK | | | | | | |
| | OS = Homo sapiens GN = NUCKS1 | | | | | | | |
| | PE = 1 SV = 1 | | | | | | | |
| OCAD1_HUMAN | OCIA domain-containing protein 1 | RSS[166.9984]PPGHYYQK | 3.33 | 0.0140 | −3.33 | 0.0510 | −1.00 | 0.9120 |
| | OS = Homo sapiens GN = OCIAD1 | | | | | | | |
| | PE = 1 SV = 1 | | | | | | | |
| ODPA_HUMAN | ODPA_HUMAN | YGM[147.0354]GT[181.014]S | 123.00 | 0.0000 | −61.57 | 0.0000 | 2.00 | 0.7030 |
| | | VER | | | | | | |
| ODBA_HUMAN | 2-oxoisovalerate dehydrogenase | IGHHS[166.9984]TSDDSSAY[243.0297] | −1.36 | 0.8270 | 1.52 | 0.6510 | 1.11 | 0.9090 |
| | subunit alpha, mitochondrial | RSVDEVNYWDK | | | | | | |
| | OS = Homo sapiens | GN = BCKDHA | | | | | | |
| | PE = 1 SV = 2 | | | | | | | |
| ODBA_HUMAN | 2-oxoisovalerate dehydrogenase | IGHHS[166.9984]TSDDSSAYR | 1.19 | 0.9110 | −2.72 | 0.3110 | −2.29 | 0.3270 |
| | subunit alpha, mitochondrial | | | | | | | |
| | OS = Homo sapiens GN = BCKDHA | | | | | | | |
| | PE = 1 SV = 2 | | | | | | | |
| ODBA_HUMAN | 2-oxoisovalerate dehydrogenase | IGHHS[166.9984]TSDDSSAYR | −1.52 | 0.7690 | 1.68 | 0.6920 | 1.10 | 0.9520 |
| | subunit alpha, mitochondrial | S[166.9984]VDEVNYWDK | | | | | | |
| | OS = Homo sapiens GN = BCKDHA | | | | | | | |
| | PE = 1 SV = 2 | | | | | | | |
| ODBA_HUMAN | 2-oxoisovalerate dehydrogenase | IGHHSTSDDS[166.9984]SAY[243.0297] | −1.22 | 0.8690 | 1.68 | 0.3570 | 1.38 | 0.6760 |
| | subunit alpha, mitochondrial | RSVDEVNYWDKQDH | | | | | | |
| | OS = Homo sapiens GN = BCKDHA | PISR | | | | | | |
| | PE = 1 SV = 2 | | | | | | | |
| ODBA_HUMAN | 2-oxoisovalerate dehydrogenase | S[166.9984]VDEVNYWDK | −2.64 | 0.0390 | 3.07 | 0.2160 | 1.16 | 0.9600 |
| | subunit alpha, mitochondrial | | | | | | | |
| | OS = Homo sapiens GN = BCKDHA | | | | | | | |
| | PE = 1 SV = 2 | | | | | | | |
| ODBA_HUMAN | 2-oxoisovalerate dehydrogenase | S[166.9984]VDEVNYWDKQD | 1.07 | 0.8910 | 1.08 | 0.9980 | 1.15 | 0.9130 |
| | subunit alpha, mitochondrial HPISR | | | | | | | |
| | OS = Homo sapiens GN = BCKDHA | | | | | | | |
| | PE = 1 SV = 2 | | | | | | | |
| ODPA_HUMAN | Pyruvate dehydrogenase E1 | YHGHS[166.9984]M[147.0354] | 32.46 | 0.0000 | −32.49 | 0.0000 | −1.00 | 0.9390 |
| | component subunit alpha, somatic | SDPGVS[166.9984]YR | | | | | | |
| | form, mitochondrial OS = Homo | | | | | | | |
| | sapiens GN = PDHA1 PE = 1 SV = 3 | | | | | | | |
| ODPA_HUMAN | Pyruvate dehydrogenase E1 | YHGHS[166.9984]M[147.0354] | 25.87 | 0.0000 | −27.30 | 0.0000 | −1.06 | 0.9110 |
| | component subunit alpha, somatic | SDPGVSY[243.0297]R | | | | | | |
| | form, mitochondrial OS = Homo | | | | | | | |
| | sapiens GN = PDHA1 PE = 1 SV = 3 | | | | | | | |
| ODPA_HUMAN | Pyruvate dehydrogenase E1 | YHGHS[166.9984]M[147.0354] | 1.16 | 0.5740 | −1.15 | 0.6460 | 1.01 | 0.9390 |
| | component subunit alpha, somatic | SDPGVSYR | | | | | | |
| | form, mitochondrial OS = Homo | | | | | | | |
| | sapiens GN = PDHA1 PE = 1 SV = 3 | | | | | | | |
| ODPA_HUMAN | Pyruvate dehydrogenase E1 | YHGHS[166.9984]MSDPGVSYR | −2.20 | 0.5910 | 1.89 | 0.6400 | −1.17 | 0.9490 |
| | component subunit alpha, somatic | | | | | | | |
| | form, mitochondrial OS = Homo | | | | | | | |
| | sapiens GN = PDHA1 PE = 1 SV = 3 | | | | | | | |
| ODPA_HUMAN | Pyruvate dehydrogenase E1 | YHGHSMS[166.9984]DPGVSYR | −1.64 | 0.9040 | 1.56 | 0.8440 | −1.05 | 0.9680 |
| | component subunit alpha, somatic | | | | | | | |
| | form, mitochondrial OS = Homo | | | | | | | |
| | sapiens GN = PDHA1 PE = 1 SV = 3 | | | | | | | |
| OR5K3_HUMAN | Olfactory receptor 5K3 OS = Homo | LHTPM[147.0354]Y[243.0297] | −1.79 | 0.1270 | 1.37 | 0.4930 | −1.31 | 0.7030 |
| | sapiens GN = OR5K3 PE = 2 SV = 1 | IFLGNLVLMDS[166.9984]C[160.0307] | | | | | | |
| | | C[160.0307]S[166.9984] | | | | | | |
| | | S[166.9984]AIT[181.014] | | | | | | |
| | | PKM[147.0354]LENFFSEDKR | | | | | | |
| P3C2A_HUMAN | Phosphatidylinositol-4-phosphate | TKFPY[243.0297]TNHRT[181.014] | −1.83 | 0.4560 | 5.97 | 0.0000 | 3.27 | 0.0200 |
| | 3-kinase C2 domain-containing | NPGY[243.0297]LLSPVTA | | | | | | |
| | alpha polypeptide OS = Homo | QR | | | | | | |
| | sapiens GN = PIK3C2A PE = 1 SV = 2 | | | | | | | |
| P3C2G_HUMAN | Phosphatidylinositol-4-phosphate | DLNHY[243.0297]MEQILNVS | −1.06 | 0.9200 | 1.39 | 0.7760 | 1.31 | 0.8250 |
| | 3-kinase C2 domain-containing | HEVTNSDC[160.0307]VLSFFL | | | | | | |
| | gamma polypeptide OS = Homo | S[166.9984]EAVQQT[181.014] | | | | | | |
| | sapiens GN = PIK3C2G PE = 1 SV = 1 | VES[166.9984]SPVYLGEK | | | | | | |
| PACN3_HUMAN | Protein kinase C and casein kinase | DGTAPPPQSPGSPGTGQDEEW | 1.38 | 0.5740 | −1.02 | 0.9590 | 1.35 | 0.6040 |
| | substrate in neurons protein 3 | S[166.9984]DEESPRK | | | | | | |
| | OS = Homo sapiens GN = PACSIN3 | | | | | | | |
| | PE = 1 SV = 2 | | | | | | | |
| PACN3_HUMAN | Protein kinase C and casein kinase | GGRS[166.9984]PDEVTLTSIVP | −1.01 | 0.9800 | 1.13 | 0.9500 | 1.12 | 0.9280 |
| | substrate in neurons protein 3 | TR | | | | | | |
| | OS = Homo sapiens | GN = PACSIN3 | | | | | | |
| | PE = 1 SV = 2 | | | | | | | |
| PALMD_HUMAN | Palmdelphin OS = Homo sapiens | NS[166.9984]KSPTEYHEPVYA | 1.79 | 0.4170 | −1.55 | 0.6290 | 1.15 | 0.8180 |
| | GN = PALMD PE = 1 SV = 1 | NPFYRPTTPQR | | | | | | |
| PALMD_HUMAN | Palmdelphin OS = Homo sapiens | SEHQNS[166.9984]SPTC[160.0307] | 1.22 | 0.7960 | −1.03 | 0.9500 | 1.18 | 0.8670 |
| | GN = PALMD PE = 1 SV = 1 | QEDEEDVR | | | | | | |
| PALMD_HUMAN | Palmdelphin OS = Homo sapiens | SPTEYHEPVYANPFYRPTT[181.014] | 2.05 | 0.0300 | −2.10 | 0.1110 | −1.02 | 0.9510 |
| | GN = PALMD PE = 1 SV = 1 | PQR | | | | | | |
| PARD3_HUMAN | Partitioning-defective 3 homolog | ENHADLGIFVKS[166.9984]IIN | 1.55 | 0.5980 | −1.46 | 0.6650 | 1.06 | 0.9770 |
| | OS = Homo sapiens GN = PARD3 | GGAAS[166.9984]KDGR | | | | | | |
| | PE = 1 SV = 2 | | | | | | | |
| PCDH1_HUMAN | Protocadherin-1 OS = Homo sapiens | SNSPLPSIQLQPQSPSAS[166.9984] | 2.11 | 0.3190 | −4.46 | 0.0090 | −2.11 | 0.0540 |
| | GN = PCDH1 PE = 1 SV = 2 | KK | | | | | | |
| PCDH7_HUMAN | Protocadherin-7 OS = Homo sapiens | LSDS[166.9984]PSM[147.0354] | 6.23 | 0.0410 | −8.23 | 0.1420 | −1.32 | 0.9020 |
| | GN = PCDH7 PE = 1 SV = 1 | GR | | | | | | |
| PCNP_HUMAN | PEST proteolytic signal-containing | NIGRDT[181.014]PTSAGPNSF | 1.95 | 0.2450 | −2.53 | 0.0810 | −1.30 | 0.2920 |
| | nuclear protein OS = Homo sapiens | NK | | | | | | |
| | GN = PCNP PE = 1 SV = 2 | | | | | | | |
| PCY1A_HUMAN | Choline-phosphate | SPS[166.9984]PSFR | −1.65 | 0.6710 | 2.27 | 0.2970 | 1.37 | 0.7610 |
| | cytidylyltransferase A OS = Homo | | | | | | | |
| | sapiens GN = PCYT1A PE = 1 SV = 2 | | | | | | | |
| PCY1A_HUMAN | Choline-phosphate | T[181.014]SPPC[160.0307]SP | −1.01 | 0.9060 | −1.43 | 0.6950 | −1.44 | 0.4740 |
| | cytidylyltransferase A OS = Homo | ANLSR | | | | | | |
| | sapiens GN = PCYT1A PE = 1 SV = 2 | | | | | | | |
| PCY1B_HUMAN | Choline-phosphate | MLQALS[166.9984]PK | 1.11 | 0.9060 | 1.50 | 0.7390 | 1.66 | 0.6030 |
| | cytidylyltransferase B OS = Homo | | | | | | | |
| | sapiens GN = PCYT1B PE = 1 SV = 1 | | | | | | | |
| PDCD4_HUMAN | Programmed cell death protein 4 | SGLTVPTS[166.9984]PK | 2.02 | 0.4720 | 1.11 | 0.9370 | 2.23 | 0.2830 |
| | OS = Homo sapiens GN = PDCD4 | | | | | | | |
| | PE = 1 SV = 1 | | | | | | | |
| PDE1C_HUMAN | Calcium/calmodulin-dependent | RSS[166.9984]LNSISSSDAK | 1.10 | 0.9680 | 1.14 | 0.9060 | 1.26 | 0.8690 |
| | 3′,5′-cyclic nucleotide | | | | | | | |
| | phosphodiesterase 1C OS = Homo | | | | | | | |
| | sapiens GN = PDE1C PE = 2 SV = 1 | | | | | | | |
| PDE3A_HUMAN | cGMP-inhibited 3′,5′-cyclic | RTS[166.9984]LPC[160.0307] | 1.79 | 0.5740 | 1.40 | 0.8510 | 2.51 | 0.2940 |
| | phosphodiesterase A OS = Homo | IPR | | | | | | |
| | sapiens GN = PDE3A PE = 1 SV = 3 | | | | | | | |
| PDLI2_HUMAN | PDZ and LIM domain protein 2 | SSRPSM[147.0354]DSEGGS[166.9984] | 1.18 | 0.7690 | −1.29 | 0.6790 | −1.10 | 0.9600 |
| | OS = Homo sapiens GN = PDLIM2 | LLLDEDSEVFKMLQE | | | | | | |
| | PE = 1 SV = 1 | NR | | | | | | |
| PEBP1_HUMAN | Phosphatidylethanolamine- | NRPTS[166.9984]ISWDGLDS | −1.00 | 0.9980 | −1.14 | 0.7420 | −1.14 | 0.7340 |
| | binding protein 1 - Homo sapiens | GK | | | | | | |
| | (Human) | | | | | | | |
| PEBP1_HUMAN | Phosphatidylethanolamine- | NRPTSIS[166.9984]WDGLDS | −1.01 | 0.9790 | −1.96 | 0.3180 | −1.99 | 0.1520 |
| | binding protein 1 - Homo sapiens | GK | | | | | | |
| | (Human) | | | | | | | |
| PGM1_HUMAN | Phosphoglucomutase-1 OS = Homo | AIGGIILTAS[166.9984]HNPGG | −1.21 | 0.8970 | −1.70 | 0.6240 | −2.06 | 0.2780 |
| | sapiens GN = PGM1 PE = 1 SV = 3 | PNGDFGIK | | | | | | |
| PGM2_HUMAN | Phosphoglucomutase-2 OS = Homo | KAVLPTS[166.9984]K | 1.45 | 0.6870 | −1.51 | 0.7040 | −1.04 | 0.9910 |
| | sapiens GN = PGM2 PE = 1 SV = 4 | | | | | | | |
| PGM2_HUMAN | Phosphoglucomutase-2 OS = Homo | SALKDTY[243.0297]M[147.0354] | 1.74 | 0.5540 | 1.25 | 0.7030 | 2.16 | 0.1050 |
| | sapiens GN = PGM2 PE = 1 SV = 4 | LSST[181.014]VS[166.9984] | | | | | | |
| | | SKILR | | | | | | |
| PGM52_HUMAN | Putative PGMS-like protein 2 | AAGGIILTAS[166.9984]HC[160.0307] | 1.15 | 0.9250 | −1.17 | 0.9350 | −1.02 | 0.9770 |
| | OS = Homo sapiens PE = 5 SV = 2 | PGGPGGEFGVK | | | | | | |
| PGRC1_HUMAN | Membrane-associated | EGEEPTVYS[166.9984]DEEEP | 1.33 | 0.3730 | 1.09 | 0.9350 | 1.45 | 0.2670 |
| | progesterone receptor component | KDESAR | | | | | | |
| | 1 - Homo sapiens (Human) | | | | | | | |
| PGRC1_HUMAN | Membrane-associated | LLKEGEEPTVYS[166.9984]DEE | 2.22 | 0.0590 | −1.43 | 0.7060 | 1.55 | 0.1000 |
| | progesterone receptor component | EPKDESAR | | | | | | |
| | 1 - Homo sapiens (Human) | | | | | | | |
| PGRC2_HUMAN | Membrane-associated | LLKPGEEPS[166.9984]EYTDEE | 1.32 | 0.1580 | −1.22 | 0.4020 | 1.08 | 0.9270 |
| | progesterone receptor component | DTKDHNKQD | | | | | | |
| | 2 - Homo sapiens (Human) | | | | | | | |
| PGRC2_HUMAN | Membrane-associated | LLKPGEEPSEYT[181.014]DEE | −1.17 | 0.8670 | 1.25 | 0.8010 | 1.07 | 0.9500 |
| | progesterone receptor component | DTK | | | | | | |
| | 2 - Homo sapiens (Human) | | | | | | | |
| PGRC2_HUMAN | Membrane-associated | LLKPGEEPSEYT[181.014]DEE | −1.53 | 0.4490 | 1.46 | 0.6270 | −1.05 | 0.9040 |
| | progesterone receptor component | DTKDHNK | | | | | | |
| | 2 - Homo sapiens (Human) | | | | | | | |
| PGRC2_HUMAN | Membrane-associated | LLKPGEEPSEYT[181.014]DEE | 1.15 | 0.4140 | −1.13 | 0.5910 | 1.02 | 0.9820 |
| | progesterone receptor component | DTKDHNKQD | | | | | | |
| | 2 - Homo sapiens (Human) | | | | | | | |
| PHF6_HUMAN | PHD finger protein 6 OS = Homo | DRS[166.9984]PHRS[166.9984] | 1.04 | 0.8930 | 1.06 | 0.9060 | 1.11 | 0.9820 |
| | sapiens GN = PHF6 PE = 1 SV = 1 | SPSDTRPK | | | | | | |
| PININ_HUMAN | Pinin - Homo sapiens (Human) | SLS[166.9984]PGKENVSALD | 2.16 | 0.6980 | −3.54 | 0.3860 | −1.64 | 0.5600 |
| | | M[147.0354]EK | | | | | | |
| PKP2_HUMAN | Plakophilin-2 OS = Homo sapiens | RLEIS[166.9984]PDS[166.9984] | 1.50 | 0.3870 | −1.09 | 0.8430 | 1.37 | 0.4950 |
| | GN = PKP2 PE = 1 SV = 1 | SPER | | | | | | |
| PLCL1_HUMAN | Inactive phospholipase C-like | KTVSFS[166.9984]SMPSEKK | 2.00 | 0.2850 | −1.13 | 0.8960 | 1.76 | 0.6620 |
| | protein 1 OS = Homo sapiens | | | | | | | |
| | GN = PLCL1 PE = 1 SV = 2 | | | | | | | |
| PLEC1_HUMAN | Plectin-1 - Homo sapiens (Human) | DPYSGS[166.9984]TISLFQAM | 1.32 | 0.7260 | −2.78 | 0.8320 | −2.11 | 0.3100 |
| | | [147.0354]QK | | | | | | |
| PLM_HUMAN | Phospholemman OS = Homo | TGEPDEEEGTFRSS[166.9984]IR | 1.40 | 0.8120 | −2.44 | 0.1950 | −1.75 | 0.3280 |
| | sapiens GN = FXYD1 PE = 1 SV = 2 | | | | | | | |
| PMGT1_HUMAN | Protein O-linked-mannose beta- | NPC[160.0307]EDSFLPDTEGH | 1.17 | 0.8890 | −1.87 | 0.1210 | −1.59 | 0.1390 |
| | 1,2-N- | TYVAFIRM[147.0354]EKDDDF | | | | | | |
| | acetylglucosaminyltransferase 1 | T[181.014]T[181.014]WTQLAK | | | | | | |
| | OS = Homo sapiens GN = POMGNT1 | | | | | | | |
| | PE = 1 SV = 1 | | | | | | | |
| POLH_HUMAN | DNA polymerase eta OS = Homo | RLSS[166.9984]LR | −1.04 | 0.8580 | −1.59 | 0.5710 | −1.65 | 0.2120 |
| | sapiens GN = POLH PE = 1 SV = 1 | | | | | | | |
| POPD1_HUMAN | Blood vessel epicardial substance | GTSS[166.9984]MS[166.9984] | −8.02 | 0.0120 | 2.03 | 0.9410 | −3.96 | 0.1710 |
| | OS = Homo sapiens GN = BVES PE = 2 | SLHVSSPHQR | | | | | | |
| | SV = 1 | | | | | | | |
| POPD1_HUMAN | Blood vessel epicardial substance | GTSSM[147.0354]S[166.9984] | 1.68 | 0.9360 | −2.65 | 0.7520 | −1.58 | 0.7490 |
| | OS = Homo sapiens GN = BVES PE = 2 | SLHVSSPHQR | | | | | | |
| | SV = 1 | | | | | | | |
| POPD1_HUMAN | Blood vessel epicardial substance | GTSSMSS[166.9984]LHVSSPH | −5.94 | 0.0840 | 1.35 | 0.7040 | −4.40 | 0.1280 |
| | OS = Homo sapiens GN = BVES PE = 2 | QR | | | | | | |
| | SV = 1 | | | | | | | |
| POPD1_HUMAN | Blood vessel epicardial substance | M[147.0354]KPIEEGAEDDDD | 2.59 | 0.5440 | −2.02 | 0.7040 | 1.28 | 0.8880 |
| | OS = Homo sapiens GN = BVES PE = 2 | VFEPAS[166.9984]PNTLK | | | | | | |
| | SV = 1 | | | | | | | |
| POPD1_HUMAN | Blood vessel epicardial substance | MKPIEEGAEDDDDVFEPAS[166.9984] | −1.69 | 0.7770 | 1.37 | 0.9220 | −1.24 | 0.9130 |
| | OS = Homo sapiens GN = BVES PE = 2 | PNTLK | | | | | | |
| | SV = 1 | | | | | | | |
| POPD1_HUMAN | Blood vessel epicardial substance | NS[166.9984]IASSSDSDDGLH | −10.06 | 0.0000 | 1.30 | 0.9980 | −7.72 | 0.0000 |
| | OS = Homo sapiens GN = BVES PE = 2 | QFLR | | | | | | |
| | SV = 1 | | | | | | | |
| POPD1_HUMAN | Blood vessel epicardial substance | NSIASSS[166.9984]DSDDGLH | 1.59 | 0.5750 | −2.23 | 0.0800 | −1.40 | 0.1630 |
| | OS = Homo sapiens GN = BVES PE = 2 | QFLR | | | | | | |
| | SV = 1 | | | | | | | |
| POPD1_HUMAN | Blood vessel epicardial substance | NSIASSSDS[166.9984]DDGLH | 1.41 | 0.5580 | −1.71 | 0.3880 | −1.21 | 0.7170 |
| | OS = Homo sapiens GN = BVES PE = 2 | QFLR | | | | | | |
| | SV = 1 | | | | | | | |
| POPD2_HUMAN | Popeye domain-containing protein | GQAPLAPTHT[181.014]PEL | 1.62 | 0.4390 | −1.78 | 0.3110 | −1.10 | 0.9020 |
| | 2 OS = Homo sapiens GN = POPDC2 | | | | | | | |
| | PE = 2 SV = 2 | | | | | | | |
| POPD2_HUMAN | Popeye domain-containing protein | IPLQSYS[166.9984]QVISR | −1.42 | 0.7950 | 1.06 | 0.9980 | −1.34 | 0.8250 |
| | 2 OS = Homo sapiens GN = POPDC2 | | | | | | | |
| | PE = 2 SV = 2 | | | | | | | |
| POPD2_HUMAN | Popeye domain-containing protein | LSRPDS[166.9984]GILASR | −1.34 | 0.4330 | 3.21 | 0.0050 | 2.39 | 0.0580 |
| | 2 OS = Homo sapiens GN = POPDC2 | | | | | | | |
| | PE = 2 SV = 2 | | | | | | | |
| PP16A_HUMAN | Protein phosphatase 1 regulatory | M[147.0354]S[166.9984]TQE | −2.81 | 0.0410 | 1.22 | 0.7040 | −2.30 | 0.2370 |
| | subunit 16A OS = Homo sapiens | RLKHAQK | | | | | | |
| | GN = PPP1R16A PE = 1 SV = 1 | | | | | | | |
| PPHLN_HUMAN | Periphilin-1 OS = Homo sapiens | DTS[166.9984]PSSGSAVSSSK | 1.13 | 0.7990 | 1.09 | 0.9060 | 1.24 | 0.8880 |
| | GN = PPHLN1 PE = 1 SV = 2 | | | | | | | |
| PPLA_HUMAN | Cardiac phospholamban OS = Homo | RAS[166.9984]TIEMPQQAR | −2.59 | 0.2540 | −1.24 | 0.6110 | −3.21 | 0.0660 |
| | sapiens GN = PLN PE = 1 SV = 1 | | | | | | | |
| PPLA_HUMAN | Cardiac phospholamban OS = Homo | RAST[181.014]IEM[147.0354] | 2.25 | 0.8360 | −1.13 | 0.8900 | 2.00 | 0.5600 |
| | sapiens GN = PLN PE = 1 SV = 1 | PQQAR | | | | | | |
| PPR1C_HUMAN | Protein phosphatase 1 regulatory | RGPNTQGELQNAS[166.9984] | −1.16 | 0.9090 | 1.23 | 0.9500 | 1.07 | 0.9600 |
| | subunit 1C OS = Homo sapiens | PK | | | | | | |
| | GN = PPP1R1C PE = 2 SV = 1 | | | | | | | |
| PR38B_HUMAN | Pre-mRNA-splicing factor 38B | RRS[166.9984]QS[166.9984]I | −1.16 | 0.9200 | 1.00 | 0.9480 | −1.16 | 0.8750 |
| | OS = Homo sapiens GN = PRPF38B | EQESQEK | | | | | | |
| | PE = 1 SV = 1 | | | | | | | |
| PRKRA_HUMAN | Interferon-inducible double | EDS[166.9984]GTFSLGK | −1.56 | 0.6580 | 1.20 | 0.8240 | −1.29 | 0.8250 |
| | stranded RNA-dependent protein | | | | | | | |
| | kinase activator A OS = Homo | | | | | | | |
| | sapiens GN = PRKRA PE = 1 SV = 1 | | | | | | | |
| PRKRA_HUMAN | Interferon-inducible double | HRAEAPPLEREDS[166.9984]G | 1.63 | 0.6990 | −1.08 | 0.9450 | 1.51 | 0.7290 |
| | stranded RNA-dependent protein | TFSLGK | | | | | | |
| | kinase activator A OS = Homo | | | | | | | |
| | sapiens GN = PRKRA PE = 1 SV = 1 | | | | | | | |
| PRP4B_HUMAN | Serine/threonine-protein kinase | KKS[166.9984]PIINESR | 1.98 | 0.4970 | −2.35 | 0.3170 | −1.19 | 0.6760 |
| | PRP4 homolog OS = Homo sapiens | | | | | | | |
| | GN = PRPF4B PE = 1 SV = 2 | | | | | | | |
| PRP4B_HUMAN | Serine/threonine-protein kinase | RRS[166.9984]LS[166.9984]P | 1.40 | 0.9800 | −1.09 | 0.8530 | 1.29 | 0.7650 |
| | PRP4 homolog OS = Homo sapiens | KPR | | | | | | |
| | GN = PRPF4B PE = 1 SV = 2 | | | | | | | |
| PRP4B_HUMAN | Serine/threonine-protein kinase | S[166.9984]RS[166.9984]PLL | 1.03 | 0.9210 | −1.31 | 0.6240 | −1.27 | 0.7550 |
| | PRP4 homolog OS = Homo sapiens | NDR | | | | | | |
| | GN = PRPF4B PE = 1 SV = 2 | | | | | | | |
| PRP4B_HUMAN | Serine/threonine-protein kinase | S[166.9984]RS[166.9984]PLL | 1.03 | 0.9370 | −1.55 | 0.6460 | −1.51 | 0.5600 |
| | PRP4 homolog OS = Homo sapiens | NDRR | | | | | | |
| | GN = PRPF4B PE = 1 SV = 2 | | | | | | | |
| PSA3_HUMAN | Proteasome subunit alpha type-3 - | ESLKEEDES[166.9984]DDDNM | −1.96 | 0.9360 | 1.26 | 0.8020 | −1.56 | 0.8160 |
| | Homo sapiens (Human) | | | | | | | |
| PSA3_HUMAN | Proteasome subunit alpha type-3 - | ESLKEEDES[166.9984]DDDN | 1.41 | 0.9040 | −1.64 | 0.9800 | −1.17 | 0.8160 |
| | Homo sapiens (Human) | M[147.0354] | | | | | | |
| PTN13_HUMAN | Tyrosine-protein phosphatase non- | AIST[181.014]GSLASS[166.9984] | 2.22 | 0.0040 | 1.01 | 0.9150 | 2.24 | 0.0300 |
| | receptor type 13 OS = Homo | TLNK | | | | | | |
| | sapiens GN = PTPN13 PE = 1 SV = 2 | | | | | | | |
| PTPRG_HUMAN | Receptor-type tyrosine-protein | VGLAPLPGM[147.0354]KGT[181.014] | 1.39 | 0.5520 | −1.02 | 0.9960 | 1.36 | 0.4980 |
| | phosphatase gamma OS = Homo | DYINAS[166.9984]Y[243.0297] | | | | | | |
| | sapiens GN = PTPRG PE = 1 SV = 3 | IMGYY[243.0297]R | | | | | | |
| PTRF_HUMAN | Polymerase I and transcript | ES[166.9984]EALPEKEGEELGE | −1.12 | 0.9990 | 1.12 | 0.8590 | −1.00 | 0.9020 |
| | release factor OS = Homo sapiens | GERPEEDAAALELS[166.9984] | | | | | | |
| | GN = PTRF PE = 1 SV = 1 | SDEAVEVEEVIEESR | | | | | | |
| PTRF_HUMAN | Polymerase I and transcript | ES[166.9984]EALPEKEGEELGE | −1.04 | 0.9840 | −1.03 | 0.9740 | −1.06 | 0.9990 |
| | release factor OS = Homo sapiens | GERPEEDAAALELSS[166.9984] | | | | | | |
| | GN = PTRF PE = 1 SV = 1 | DEAVEVEEVIEESR | | | | | | |
| PTRF_HUMAN | Polymerase I and transcript | KVS[166.9984]VNVK | 2.15 | 0.2220 | −2.38 | 0.2260 | −1.11 | 0.9380 |
| | release factor OS = Homo sapiens | | | | | | | |
| | GN = PTRF PE = 1 SV = 1 | | | | | | | |
| PTRF_HUMAN | Polymerase I and transcript | LPAKLS[166.9984]ISK | 1.27 | 0.1300 | −1.18 | 0.6540 | 1.08 | 0.8640 |
| | release factor OS = Homo sapiens | | | | | | | |
| | GN = PTRF PE = 1 SV = 1 | | | | | | | |
| PTRF_HUMAN | Polymerase I and transcript | RGS[166.9984]S[166.9984]P | 1.53 | 0.5180 | −1.10 | 0.9190 | 1.38 | 0.5790 |
| | release factor OS = Homo sapiens | DVHALLEITEESDAVLVDK | | | | | | |
| | GN = PTRF PE = 1 SV = 1 | | | | | | | |
| PTRF_HUMAN | Polymerase I and transcript | S[166.9984]FTPDHVVYAR | 1.08 | 0.8880 | 1.54 | 0.2700 | 1.67 | 0.1130 |
| | release factor OS = Homo sapiens | | | | | | | |
| | GN = PTRF PE = 1 SV = 1 | | | | | | | |
| PTRF_HUMAN | Polymerase I and transcript | S[166.9984]LKESEALPEK | −1.25 | 0.8150 | 1.39 | 0.7030 | 1.11 | 0.9070 |
| | release factor OS = Homo sapiens | | | | | | | |
| | GN = PTRF PE = 1 SV = 1 | | | | | | | |
| PTRF_HUMAN | Polymerase I and transcript | VM[147.0354]IYQDEVKLPAKL | 2.28 | 0.6310 | −2.22 | 0.6460 | 1.03 | 0.9230 |
| | release factor OS = Homo sapiens | SIS[166.9984]K | | | | | | |
| | GN = PTRF PE = 1 SV = 1 | | | | | | | |
| PTRFL_HUMAN | PTRF/SDPR family protein | EIPT[181.014]PEPLK | 9.33 | 0.0000 | −2.60 | 0.0120 | 3.59 | 0.1390 |
| | OS = Homo sapiens PE = 2 SV = 2 | | | | | | | |
| PTRFL_HUMAN | PTRF/SDPR family protein | LS[166.9984]SVTEDEDQDAAL | −1.58 | 0.6560 | −1.27 | 0.8490 | −2.01 | 0.4930 |
| | OS = Homo sapiens PE = 2 SV = 2 | TIVTVLDK | | | | | | |
| PTRFL_HUMAN | PTRF/SDPR family protein | S[166.9984]ESLGPISELYSDELS | −1.20 | 0.9630 | 2.34 | 0.0520 | 1.94 | 0.1390 |
| | OS = Homo sapiens PE = 2 SV = 2 | EPEHEAARPVYPPHEGR | | | | | | |
| PTRFL_HUMAN | PTRF/SDPR family protein | S[166.9984]GKEHIDNIK | −1.41 | 0.6370 | 1.89 | 0.0270 | 1.33 | 0.4410 |
| | OS = Homo sapiens PE = 2 SV = 2 | | | | | | | |
| PTRFL_HUMAN | PTRF/SDPR family protein | S[166.9984]GKEHIDNIKK | −1.44 | 0.6360 | 2.00 | 0.0420 | 1.39 | 0.4150 |
| | OS = Homo sapiens PE = 2 SV = 2 | | | | | | | |
| PTRFL_HUMAN | PTRF/SDPR family protein | SES[166.9984]LGPISELYSDELS | −1.07 | 0.9370 | −1.31 | 0.7240 | −1.39 | 0.6750 |
| | OS = Homo sapiens PE = 2 SV = 2 | [166.9984]EPEHEAARPVYPPH | | | | | | |
| | | EGR | | | | | | |
| PTRFL_HUMAN | PTRF/SDPR family protein | SES[166.9984]LGPISELYSDELS | −1.67 | 0.5680 | 3.68 | 0.0000 | 2.20 | 0.0270 |
| | OS = Homo sapiens PE = 2 SV = 2 | EPEHEAARPVYPPHEGR | | | | | | |
| PXDC2_HUMAN | Plexin domain-containing protein 2 | RGS[166.9984]GHPAYAEVEPV | 1.36 | 0.8360 | 3.15 | 0.0420 | 4.28 | 0.0070 |
| | OS = Homo sapiens GN = PLXDC2 | GEK | | | | | | |
| | PE = 1 SV = 1 | | | | | | | |
| QCR6_HUMAN | Cytochrome b-c1 complex subunit | S[166.9984]HTEEDC[160.0307] | −1.38 | 0.8780 | −1.30 | 0.8180 | −1.80 | 0.6930 |
| | 6, mitochondrial precursor - Homo | TEELFDFLHAR | | | | | | |
| | sapiens (Human) | | | | | | | |
| QCR6_HUMAN | Cytochrome b-c1 complex subunit | SHT[181.014]EEDC[160.0307] | −1.08 | 0.9800 | −1.81 | 0.4630 | −1.95 | 0.7030 |
| | 6, mitochondrial precursor - Homo | TEELFDFLHAR | | | | | | |
| | sapiens (Human) | | | | | | | |
| QSOX2_HUMAN | Sulfhydryl oxidase 2 OS = Homo | QHYGRDNLLDTYSADQGDS[166.9984] | 1.70 | 0.7260 | −1.03 | 0.9060 | 1.66 | 0.4280 |
| | sapiens GN = QSOX2 PE = 2 SV = 3 | SEGGTLARGEEEEK | | | | | | |
| RA1L3_HUMAN | Putative heterogeneous nuclear | SES[166.9984]PKEPEQLR | 1.37 | 0.0820 | −1.14 | 0.5780 | 1.20 | 0.2930 |
| | ribonucleoprotein A1-like protein | | | | | | | |
| | 3 OS = Homo sapiens | | | | | | | |
| | GN = HNRPA1L3 PE = 5 SV = 1 | | | | | | | |
| RA1L3_HUMAN | Putative heterogeneous nuclear | SES[166.9984]PKEPEQLRK | 2.48 | 0.2880 | −1.62 | 0.6810 | 1.53 | 0.4280 |
| | ribonucleoprotein A1-like protein | | | | | | | |
| | 3 OS = Homo sapiens | | | | | | | |
| | GN = HNRPA1L3 PE = 5 SV = 1 | | | | | | | |
| RAD_HUMAN | GTP-binding protein RAD | RGS[166.9984]TPWGPAPPLHR | 1.04 | 0.6380 | 1.04 | 0.9150 | 1.08 | 0.4750 |
| | OS = Homo sapiens GN = RRAD PE = 1 | | | | | | | |
| | SV = 2 | | | | | | | |
| RAD_HUMAN | GTP-binding protein RAD | RRGST[181.014]PWGPAPPLHR | 1.85 | 0.9250 | −1.58 | 0.8490 | 1.17 | 0.7380 |
| | OS = Homo sapiens GN = RRAD PE = 1 | | | | | | | |
| | SV = 2 | | | | | | | |
| RAD_HUMAN | GTP-binding protein RAD | SKS[166.9984]C[160.0307]H | 1.89 | 0.0230 | −1.51 | 0.3360 | 1.25 | 0.7260 |
| | OS = Homo sapiens GN = RRAD PE = 1 | DLSVL | | | | | | |
| | SV = 2 | | | | | | | |
| RALY_HUMAN | RNA-binding protein Raly | GRLS[166.9984]PVPVPR | 2.58 | 0.2150 | −1.77 | 0.5000 | 1.45 | 0.6650 |
| | OS = Homo sapiens GN = RALY PE = 1 | | | | | | | |
| | SV = 1 | | | | | | | |
| RBBP6_HUMAN | Retinoblastoma-binding protein 6 - | LEVTEIVKPS[166.9984]PK | 1.57 | 0.4660 | −1.67 | 0.4350 | −1.06 | 0.8840 |
| | Homo sapiens (Human) | | | | | | | |
| RBM10_HUMAN | RNA-binding protein 10 OS = Homo | LASDDRPS[166.9984]PPR | 2.08 | 0.2580 | −1.31 | 0.8020 | 1.59 | 0.6300 |
| | sapiens GN = RBM10 PE = 1 SV = 3 | | | | | | | |
| RBM25_HUMAN | Probable RNA-binding protein 25 | LGASNS[166.9984]PGQPNSVK | 2.27 | 0.0680 | −1.56 | 0.4930 | 1.46 | 0.7230 |
| | OS = Homo sapiens GN = RBM25 | | | | | | | |
| | PE = 1 SV = 2 | | | | | | | |
| RBM39_HUMAN | RNA-binding protein 39 - Homo | DKS[166.9984]PVREPIDNLTP | 1.57 | 0.5180 | −1.29 | 0.7520 | 1.22 | 0.6940 |
| | sapiens (Human) | EER | | | | | | |
| RBP1_HUMAN | RalA-binding protein 1 OS = Homo | AGKEPAKPS[166.9984]PSR | 2.30 | 0.1720 | 1.22 | 0.6240 | 2.81 | 0.0210 |
| | sapiens GN = RALBP1 PE = 1 SV = 3 | | | | | | | |
| RCAS1_HUMAN | Receptor-binding cancer antigen | KLSGDQIT[181.014]LPTTVDY | 1.23 | 0.6340 | −1.80 | 0.0850 | −1.46 | 0.4280 |
| | expressed on SiSo cells OS = Homo | SSVPK | | | | | | |
| | sapiens GN = EBAG9 PE = 1 SV = 1 | | | | | | | |
| RFPL1_HUMAN | Ret finger protein-like 1 OS = Homo | FQVDMTLDADT[181.014]AN | 1.72 | 0.8640 | −2.33 | 0.6280 | −1.36 | 0.5530 |
| | sapiens GN = RFPL1 PE = 2 SV = 2 | NFLLISDDLR | | | | | | |
| RHG22_HUMAN | Rho GTPase-activating protein 22 | S[166.9984]LDLDHSM[147.0354] | 1.03 | 0.9720 | 1.22 | 0.7920 | 1.25 | 0.7140 |
| | OS = Homo sapiens GN = ARHGAP22 | DEAGAGASNSEPSEPDSP | | | | | | |
| | PE = 2 SV = 1 | TREHAR | | | | | | |
| RIC3_HUMAN | Protein RIC-3 OS = Homo sapiens | SHLAEAFAKAKGS[166.9984]G | −1.11 | 0.8440 | 1.24 | 0.8060 | 1.11 | 0.9560 |
| | GN = RIC3 PE = 1 SV = 1 | GGAGGGGS[166.9984]GR | | | | | | |
| RL1D1_HUMAN | Ribosomal L1 domain-containing | AT[181.014]NESEDEIPQLVPIGK | 1.49 | 0.3720 | −1.58 | 0.3430 | −1.06 | 0.9520 |
| | protein 1 OS = Homo sapiens | | | | | | | |
| | GN = RSL1D1 PE = 1 SV = 3 | | | | | | | |
| RL1D1_HUMAN | Ribosomal L1 domain-containing | ATNES[166.9984]EDEIPQLVPI | 1.21 | 0.7450 | −1.23 | 0.7040 | −1.01 | 0.9820 |
| | protein 1 OS = Homo sapiens | GK | | | | | | |
| | GN = RSL1D1 PE = 1 SV = 3 | | | | | | | |
| RLA2_HUMAN | 60S acidic ribosomal protein P2 | KEES[166.9984]EES[166.9984] | 3.57 | 0.5310 | −5.06 | 0.3020 | −1.42 | 0.8470 |
| | OS = Homo sapiens GN = RPLP2 PE = 1 | DDDM[147.0354]GFGLFD | | | | | | |
| | SV = 1 | | | | | | | |
| RLA2_HUMAN | 60S acidic ribosomal protein P2 | KEES[166.9984]EES[166.9984] | −3.84 | 0.1110 | 1.35 | 0.9350 | −2.84 | 0.3780 |
| | OS = Homo sapiens GN = RPLP2 PE = 1 | DDDMGFGLFD | | | | | | |
| | SV = 1 | | | | | | | |
| RMP_HUMAN | Unconventional prefoldin RPB5 | KNS[166.9984]TGSGHSAQELP | −1.30 | 0.8270 | −1.03 | 0.9500 | −1.35 | 0.7520 |
| | interactor OS = Homo sapiens | TIR | | | | | | |
| | GN = RMP PE = 1 SV = 2 | | | | | | | |
| RNPS1_HUMAN | RNA-binding protein with serine- | RFS[166.9984]PPR | 1.43 | 0.6670 | −1.21 | 0.8020 | 1.18 | 0.9510 |
| | rich domain 1 OS = Homo sapiens | | | | | | | |
| | GN = RNPS1 PE = 1 SV = 1 | | | | | | | |
| ROA3_HUMAN | Heterogeneous nuclear | SSGS[166.9984]PYGGGYGSG | 1.53 | 0.4870 | −1.19 | 0.7960 | 1.29 | 0.7370 |
| | ribonucleoprotein A3 - Homo | GGSGGYGSR | | | | | | |
| | sapiens (Human) | | | | | | | |
| RP1_HUMAN | Oxygen-regulated protein 1 | S[166.9984]VIGS[166.9984]V | −1.57 | 0.1170 | −1.45 | 0.7970 | −2.28 | 0.0070 |
| | OS = Homo sapiens GN = RP1 PE = 1 | TLVSETEVQEK | | | | | | |
| | SV = 1 | | | | | | | |
| RRAS2_HUMAN | Ras-related protein R-Ras2 | KFQEQEC[160.0307]PPS[166.9984] | −1.05 | 0.9200 | 1.92 | 0.4150 | 1.83 | 0.5550 |
| | OS = Homo sapiens GN = RRAS2 | PEPTR | | | | | | |
| | PE = 1 SV = 1 | | | | | | | |
| RRAS2_HUMAN | Ras-related protein R-Ras2 | KFQEQEC[160.0307]PPS[166.9984] | 1.68 | 0.2230 | 1.26 | 0.9000 | 2.11 | 0.3690 |
| | OS = Homo sapiens GN = RRAS2 | PEPTRK | | | | | | |
| | PE = 1 SV = 1 | | | | | | | |
| RSRC2_HUMAN | Arginine/serine-rich coiled-coil | EQSEVSVS[166.9984]PR | 1.39 | 0.6560 | −1.56 | 0.4460 | −1.12 | 0.8160 |
| | protein 2 OS = Homo sapiens | | | | | | | |
| | GN = RSRC2 PE = 1 SV = 1 | | | | | | | |
| RTKN_HUMAN | Rhotekin OS = Homo sapiens | LSSSLGRS[166.9984]SGR | 1.31 | 0.7070 | 1.23 | 0.9600 | 1.61 | 0.6750 |
| | GN = RTKN PE = 1 SV = 2 | | | | | | | |
| RTN1_HUMAN | Reticulon-1 OS = Homo sapiens | DTDIS[166.9984]IKPEGVR | 2.91 | 0.0004 | −3.14 | 0.0005 | −1.08 | 0.9560 |
| | GN = RTN1 PE = 1 SV = 1 | | | | | | | |
| RU17_HUMAN | U1 small nuclear | YDERPGPS[166.9984]PLPHR | 2.17 | 0.2790 | −1.30 | 0.7920 | 1.68 | 0.3300 |
| | ribonucleoprotein 70 kDa - Homo | | | | | | | |
| | sapiens (Human) | | | | | | | |
| S12A4_HUMAN | Solute carrier family 12 member 4 | M[147.0354]HTAVKLNEVIVTR | 1.16 | 0.9200 | 1.55 | 0.0000 | 1.81 | 0.0080 |
| | OS = Homo sapiens GN = SLC12A4 | S[166.9984]HDAR | | | | | | |
| | PE = 1 SV = 2 | | | | | | | |
| S38A1_HUMAN | Sodium-coupled neutral amino | RS[166.9984]LTNSHLEK | −1.00 | 0.9800 | −1.51 | 0.7520 | −1.51 | 0.8200 |
| | acid transporter 1 OS = Homo | | | | | | | |
| | sapiens GN = SLC38A1 PE = 1 SV = 1 | | | | | | | |
| SAFB1_HUMAN | Scaffold attachment factor B1 | SVVS[166.9984]FDK | 1.43 | 0.7950 | −1.32 | 0.8230 | 1.08 | 0.9090 |
| | OS = Homo sapiens GN = SAFB PE = 1 | | | | | | | |
| | SV = 4 | | | | | | | |
| SAFB1_HUMAN | Scaffold attachment factor B1 | SVVS[166.9984]FDKVK | 1.93 | 0.2520 | −1.73 | 0.3430 | 1.11 | 0.9040 |
| | OS = Homo sapiens GN = SAFB PE = 1 | | | | | | | |
| | SV = 4 | | | | | | | |
| SC61B_HUMAN | Protein transport protein Sec61 | PGPTPS[166.9984]GTNVGSS | 1.81 | 0.4420 | −2.24 | 0.2360 | −1.24 | 0.4920 |
| | subunit beta OS = Homo sapiens | GRSPSK | | | | | | |
| | GN = SEC61B PE = 1 SV = 2 | | | | | | | |
| SC61B_HUMAN | Protein transport protein Sec61 | PGPTPSGTNVGSSGRS[166.9984] | 1.46 | 0.4430 | −1.73 | 0.2300 | −1.19 | 0.3320 |
| | subunit beta OS = Homo sapiens | PSK | | | | | | |
| | GN = SEC61B PE = 1 SV = 2 | | | | | | | |
| SDPR_HUMAN | Serum deprivation-response | EELPDENKSLEETLHT[181.014] | 1.39 | 0.9830 | 1.21 | 0.2720 | 1.68 | 0.1900 |
| | protein OS = Homo sapiens | VDLS[166.9984]SDDDLPHDEE | | | | | | |
| | GN = SDPR PE = 1 SV = 3 | ALEDSAEEKVEESR | | | | | | |
| SDPR_HUMAN | Serum deprivation-response | IS[166.9984]S[166.9984]GKS | 1.42 | 0.8340 | −1.22 | 0.9180 | 1.16 | 0.9040 |
| | protein OS = Homo sapiens | [166.9984]SPFKVSPLTFGR | | | | | | |
| | GN = SDPR PE = 1 SV = 3 | | | | | | | |
| SDPR_HUMAN | Serum deprivation-response | ISSGKS[166.9984]S[166.9984] | 1.26 | 0.9990 | 1.01 | 0.8930 | 1.27 | 0.8700 |
| | protein OS = Homo sapiens | PFKVS[166.9984]PLTFGR | | | | | | |
| | GN = SDPR PE = 1 SV = 3 | | | | | | | |
| SDPR_HUMAN | Serum deprivation-response | S[166.9984]SPFKVS[166.9984] | 1.04 | 0.9200 | 1.10 | 0.8180 | 1.14 | 0.7650 |
| | protein OS = Homo sapiens | PLTFGR | | | | | | |
| | GN = SDPR PE = 1 SV = 3 | | | | | | | |
| SDPR_HUMAN | Serum deprivation-response | SLEETLHTVDLS[166.9984]S[166.9984] | −1.12 | 0.9060 | 1.21 | 0.6270 | 1.08 | 0.8250 |
| | protein OS = Homo sapiens | DDDLPHDEEALEDS[166.9984] | | | | | | |
| | GN = SDPR PE = 1 SV = 3 | AEEKVEESR | | | | | | |
| SDPR_HUMAN | Serum deprivation-response | SLEETLHTVDLS[166.9984]S[166.9984] | −1.65 | 0.6310 | 1.40 | 0.3600 | −1.17 | 0.9930 |
| | protein OS = Homo sapiens | DDDLPHDEEALEDSA | | | | | | |
| | GN = SDPR PE = 1 SV = 3 | EEK | | | | | | |
| SDPR_HUMAN | Serum deprivation-response | SLEETLHTVDLS[166.9984]S[166.9984] | 1.06 | 0.9250 | 1.31 | 0.3170 | 1.39 | 0.2930 |
| | protein OS = Homo sapiens | DDDLPHDEEALEDSA | | | | | | |
| | GN = SDPR PE = 1 SV = 3 | EEKVEESR | | | | | | |
| SDPR_HUMAN | Serum deprivation-response | SLEETLHTVDLSS[166.9984]D | −1.02 | 0.9500 | −1.12 | 0.9980 | −1.13 | 0.9430 |
| | protein OS = Homo sapiens | DDLPHDEEALEDS[166.9984]A | | | | | | |
| | GN = SDPR PE = 1 SV = 3 | EEKVEESR | | | | | | |
| SDPR_HUMAN | Serum deprivation-response | SSPFKVS[166.9984]PLTFGR | 1.46 | 0.5790 | −2.42 | 0.0820 | −1.66 | 0.5700 |
| | protein OS = Homo sapiens | | | | | | | |
| | GN = SDPR PE = 1 SV = 3 | | | | | | | |
| SDPR_HUMAN | Serum deprivation-response | VS[166.9984]PLTFGR | 1.45 | 0.6530 | −1.21 | 0.8610 | 1.20 | 0.7290 |
| | protein OS = Homo sapiens | | | | | | | |
| | GN = SDPR PE = 1 SV = 3 | | | | | | | |
| SEC62_HUMAN | Translocation protein SEC62 | VGPGNHGTEGSGGERHS[166.9984] | −1.49 | 0.7720 | 2.27 | 0.5540 | 1.52 | 0.7320 |
| | OS = Homo sapiens GN = SEC62 PE = 1 | DTDSDR | | | | | | |
| | SV = 1 | | | | | | | |
| SEPT2_HUMAN | Septin-2 - Homo sapiens (Human) | IYHLPDAES[166.9984]DEDED | 1.05 | 0.7990 | 1.67 | 0.0420 | 1.75 | 0.0070 |
| | | FKEQTR | | | | | | |
| SFRS1_HUMAN | Splicing factor, arginine/serine-rich | VDGPRS[166.9984]PS[166.9984] | −1.36 | 0.6700 | −1.38 | 0.8640 | −1.88 | 0.3780 |
| | 1 - Homo sapiens (Human) | YGR | | | | | | |
| SFRS1_HUMAN | Splicing factor, arginine/serine-rich | VDGPRS[166.9984]PS[166.9984] | 1.34 | 0.4320 | −1.82 | 0.1040 | −1.36 | 0.3090 |
| | 1 - Homo sapiens (Human) | YGRS[166.9984]R | | | | | | |
| SFRS1_HUMAN | Splicing factor, arginine/serine-rich | VDGPRS[166.9984]PSYGR | 2.46 | 0.2270 | −3.25 | 0.0580 | −1.32 | 0.7790 |
| | 1 - Homo sapiens (Human) | | | | | | | |
| SFRS1_HUMAN | Splicing factor, arginine/serine-rich | VKVDGPRS[166.9984]PS[166.9984] | 1.30 | 0.8210 | −1.57 | 0.5730 | −1.20 | 0.8400 |
| | 1 - Homo sapiens (Human) | YGR | | | | | | |
| SFRS1_HUMAN | Splicing factor, arginine/serine-rich | VKVDGPRS[166.9984]PSY[243.0297] | 1.15 | 0.9060 | −1.25 | 0.7040 | −1.09 | 0.6910 |
| | 1 - Homo sapiens (Human) | GR | | | | | | |
| SFRS6_HUMAN | Splicing factor, arginine/serine-rich | ARS[166.9984]VS[166.9984]P | 1.18 | 0.0000 | −2.11 | 0.0000 | −1.78 | 0.4600 |
| | 6 - Homo sapiens (Human) | PPK | | | | | | |
| SFRS6_HUMAN | Splicing factor, arginine/serine-rich | ARS[166.9984]VS[166.9984]P | 1.07 | 0.9200 | −2.47 | 0.5760 | −2.30 | 0.3680 |
| | 6 - Homo sapiens (Human) | PPKR | | | | | | |
| SFRS9_HUMAN | Splicing factor, arginine/serine-rich | GS[166.9984]PHYFSPFRPY | 1.29 | 0.7000 | −1.17 | 0.7970 | 1.10 | 0.9020 |
| | 9 - Homo sapiens (Human) | | | | | | | |
| SGCA_HUMAN | Alpha-sarcoglycan OS = Homo | LPPRVDS[166.9984]AQVPLIL | −1.25 | 0.7800 | −1.05 | 0.9350 | −1.31 | 0.6750 |
| | sapiens GN = SGCA PE = 1 SV = 1 | DQH | | | | | | |
| SH3R2_HUMAN | Putative E3 ubiquitin-protein ligase | FQNYS[166.9984]PPPTK | −1.25 | 0.7070 | 1.15 | 0.7040 | −1.08 | 0.9490 |
| | SH3RF2 OS = Homo sapiens | | | | | | | |
| | GN = SH3RF2 PE = 2 SV = 2 | | | | | | | |
| SIDT2_HUMAN | SID1 transmembrane family | KDFPSNS[166.9984]FYVVVVVK | 2.06 | 0.4790 | −1.92 | 0.5250 | 1.07 | 0.8960 |
| | member 2 OS = Homo sapiens | | | | | | | |
| | GN = SIDT2 PE = 1 SV = 2 | | | | | | | |
| SLTM_HUMAN | SAFB-like transcription modulator | AGAGM[147.0354]ITQHSSNA | 1.75 | 0.7070 | −1.92 | 0.6460 | −1.10 | 0.9130 |
| | OS = Homo sapiens GN = SLTM PE = 1 | S[166.9984]PINR | | | | | | |
| | SV = 2 | | | | | | | |
| SLTM_HUMAN | SAFB-like transcription modulator | AGAGMITQHSSNAS[166.9984] | −1.22 | 0.8360 | 1.21 | 0.8180 | −1.01 | 0.9910 |
| | OS = Homo sapiens GN = SLTM PE = 1 | PINR | | | | | | |
| | SV = 2 | | | | | | | |
| SLTM_HUMAN | SAFB-like transcription modulator | DGQDAIAQS[166.9984]PEK | −1.00 | 0.9840 | −1.32 | 0.6540 | −1.32 | 0.6760 |
| | OS = Homo sapiens GN = SLTM PE = 1 | | | | | | | |
| | SV = 2 | | | | | | | |
| SLTM_HUMAN | SAFB-like transcription modulator | DGQDAIAQS[166.9984]PEKESK | 1.21 | 0.8890 | −1.59 | 0.5910 | −1.31 | 0.6620 |
| | OS = Homo sapiens GN = SLTM PE = 1 | | | | | | | |
| | SV = 2 | | | | | | | |
| SLTM_HUMAN | SAFB-like transcription modulator | S[166.9984]PGHMVILDQTK | −1.01 | 0.9350 | 1.37 | 0.7450 | 1.35 | 0.7030 |
| | OS = Homo sapiens GN = SLTM PE = 1 | | | | | | | |
| | SV = 2 | | | | | | | |
| SMAP_HUMAN | Small acidic protein OS = Homo | S[166.9984]ASPDDDLGSSNW | −1.08 | 0.9200 | −1.45 | 0.2150 | −1.56 | 0.0940 |
| | sapiens GN = SMAP PE = 1 SV = 1 | EAADLGNEER | | | | | | |
| SMAP_HUMAN | Small acidic protein OS = Homo | SAS[166.9984]PDDDLGSSNW | −1.04 | 0.9360 | −1.35 | 0.7090 | −1.40 | 0.5330 |
| | sapiens GN = SMAP PE = 1 SV = 1 | EAADLGNEERK | | | | | | |
| SMCA2_HUMAN | Probable global transcription | GRPPAEKLS[166.9984]PNPPK | 1.13 | 0.9200 | −1.07 | 0.9290 | 1.05 | 0.9820 |
| | activator SNF2L2 OS = Homo | | | | | | | |
| | sapiens GN = SMARCA2 PE = 1 SV = 1 | | | | | | | |
| SMTN_HUMAN | Smoothelin OS = Homo sapiens | STS[166.9984]FGVPNANSIK | −2.04 | 0.1100 | −1.36 | 0.7040 | −2.78 | 0.0100 |
| | GN = SMTN PE = 1 SV = 5 | | | | | | | |
| SN_HUMAN | Sialoadhesin OS = Homo sapiens | VVATSLPSGGGC[160.0307]ST | −1.21 | 0.7790 | −1.10 | 0.9980 | −1.33 | 0.7590 |
| | GN = SIGLEC1 PE = 1 SV = 2 | [181.014]C[160.0307]GGC[160.0307] | | | | | | |
| | | S[166.9984]PRM[147.0354] | | | | | | |
| | | KVTK | | | | | | |
| SNPC4_HUMAN | snRNA-activating protein complex | LASS[166.9984]RVER | −1.50 | 0.3670 | 2.43 | 0.6080 | 1.62 | 0.9260 |
| | subunit 4 OS = Homo sapiens | | | | | | | |
| | GN = SNAPC4 PE = 1 SV = 1 | | | | | | | |
| SOX4_HUMAN | Transcription factor SOX-4 | VGGS[166.9984]GGGGHGGG | −1.79 | 0.2310 | −1.21 | 0.7270 | −2.15 | 0.0410 |
| | OS = Homo sapiens GN = SOX4 PE = 1 | GGGGS[166.9984]S[166.9984] | | | | | | |
| | SV = 1 | NAGGGGGGAS[166.9984]G | | | | | | |
| | | GGANS[166.9984]KPAQK | | | | | | |
| SP100_HUMAN | Nuclear autoantigen Sp-100 | LNEC[160.0307]IS[166.9984] | 3.42 | 0.2880 | −3.39 | 0.3340 | 1.01 | 0.8840 |
| | OS = Homo sapiens GN = SP100 PE = 1 | PVANEM[147.0354]NHLPAHS | | | | | | |
| | SV = 3 | HDLQR | | | | | | |
| SP100_HUMAN | Nuclear autoantigen Sp-100 | LNEC[160.0307]IS[166.9984] | −1.12 | 0.9990 | 1.24 | 0.9150 | 1.11 | 0.9330 |
| | OS = Homo sapiens GN = SP100 PE = 1 | PVANEMNHLPAHSHDLQR | | | | | | |
| | SV = 3 | | | | | | | |
| SP100_HUMAN | Nuclear autoantigen Sp-100 | LPLQES[166.9984]EEEEREER | 1.45 | 0.2620 | 1.17 | 0.7740 | 1.70 | 0.0640 |
| | OS = Homo sapiens GN = SP100 PE = 1 | | | | | | | |
| | SV = 3 | | | | | | | |
| SPATL_HUMAN | Protein SPATIAL OS = Homo sapiens | ELKELAS[166.9984]RVAFLT[181.014]K | −3.45 | 0.0006 | 2.33 | 0.0210 | −1.48 | 0.6320 |
| | GN = SPATIAL PE = 2 SV = 2 | | | | | | | |
| SPRL1_HUMAN | SPARC-like protein 1 OS = Homo | AEDEENEKETAVS[166.9984]T | 1.27 | 0.7130 | −1.89 | 0.5020 | −1.49 | 0.4960 |
| | sapiens GN = SPARCL1 PE = 1 SV = 1 | EDDSHHK | | | | | | |
| SPRL1_HUMAN | SPARC-like protein 1 OS = Homo | DQGNQEQDPNIS[166.9984]N | 1.05 | 0.9800 | −1.29 | 0.6910 | −1.23 | 0.6690 |
| | sapiens GN = SPARCL1 PE = 1 SV = 1 | GEEEEEKEPGEVGTHNDNQER | | | | | | |
| SPRL1_HUMAN | SPARC-like protein 1 OS = Homo | HIQETEWQS[166.9984]QEGK | 1.12 | 0.9200 | −1.29 | 0.7530 | −1.15 | 0.8470 |
| | sapiens GN = SPARCL1 PE = 1 SV = 1 | | | | | | | |
| SPRL1_HUMAN | SPARC-like protein 1 OS = Homo | SSS[166.9984]QELGLK | −1.13 | 0.9200 | −1.65 | 0.5500 | −1.87 | 0.2960 |
| | sapiens GN = SPARCL1 PE = 1 SV = 1 | | | | | | | |
| SPTC2_HUMAN | Serine palmitoyltransferase 2 | KELIDY[243.0297]LR | 5.10 | 0.0040 | −2.02 | 0.4950 | 2.52 | 0.4600 |
| | OS = Homo sapiens GN = SPTLC2 | | | | | | | |
| | PE = 1 SV = 1 | | | | | | | |
| SRBS2_HUMAN | Sorbin and SH3 domain-containing | DAS[166.9984]SPVPPPHVPPP | 2.26 | 0.0040 | 1.52 | 0.3420 | 3.44 | 0.0000 |
| | protein 2 OS = Homo sapiens | VPPLRPR | | | | | | |
| | GN = SORBS2 PE = 1 SV = 3 | | | | | | | |
| SRBS2_HUMAN | Sorbin and SH3 domain-containing | DASS[166.9984]PVPPPHVPPP | 2.64 | 0.0140 | 2.64 | 0.2380 | 6.98 | 0.0007 |
| | protein 2 OS = Homo sapiens | VPPLRPR | | | | | | |
| | GN = SORBS2 PE = 1 SV = 3 | | | | | | | |
| SRBS2_HUMAN | Sorbin and SH3 domain-containing | GAEDYPDPPIPHS[166.9984]Y | −2.16 | 0.1930 | 3.77 | 0.0003 | 1.75 | 0.3710 |
| | protein 2 OS = Homo sapiens | SSDR | | | | | | |
| | GN = SORBS2 PE = 1 SV = 3 | | | | | | | |
| SRBS2_HUMAN | Sorbin and SH3 domain-containing | RKS[166.9984]EPAVGPPR | 1.77 | 0.0260 | 1.25 | 0.8020 | 2.21 | 0.0360 |
| | protein 2 OS = Homo sapiens | | | | | | | |
| | GN = SORBS2 PE = 1 SV = 3 | | | | | | | |
| SRBS2_HUMAN | Sorbin and SH3 domain-containing | S[166.9984]EPAVGPPR | 2.67 | 0.3260 | 2.68 | 0.4370 | 7.16 | 0.0180 |
| | protein 2 OS = Homo sapiens | | | | | | | |
| | GN = SORBS2 PE = 1 SV = 3 | | | | | | | |
| SRBS2_HUMAN | Sorbin and SH3 domain-containing | SFTSSS[166.9984]PS[166.9984] | 5.55 | 0.0000 | −1.69 | 0.2940 | 3.28 | 0.0350 |
| | protein 2 OS = Homo sapiens | SPSR | | | | | | |
| | GN = SORBS2 PE = 1 SV = 3 | | | | | | | |
| SRBS2_HUMAN | Sorbin and SH3 domain-containing | SFTSSSPS[166.9984]SPSR | 1.86 | 0.0810 | 1.39 | 0.3840 | 2.60 | 0.0020 |
| | protein 2 OS = Homo sapiens | | | | | | | |
| | GN = SORBS2 PE = 1 SV = 3 | | | | | | | |
| SRBS2_HUMAN | Sorbin and SH3 domain-containing | SHS[166.9984]DNSPNAFK | −1.20 | 0.9200 | 1.25 | 0.8930 | 1.04 | 0.9820 |
| | protein 2 OS = Homo sapiens | | | | | | | |
| | GN = SORBS2 PE = 1 SV = 3 | | | | | | | |
| SRBS2_HUMAN | Sorbin and SH3 domain-containing | T[181.014]SPGRVDLPGSSTTL | 3.15 | 0.0010 | −1.07 | 0.8410 | 2.95 | 0.0060 |
| | protein 2 OS = Homo sapiens | TK | | | | | | |
| | GN = SORBS2 PE = 1 SV = 3 | | | | | | | |
| SRBS2_HUMAN | Sorbin and SH3 domain-containing | TSPGRVDLPGS[166.9984]STT | 3.70 | 0.0000 | 1.04 | 0.9970 | 3.85 | 0.0004 |
| | protein 2 OS = Homo sapiens | LTK | | | | | | |
| | GN = SORBS2 PE = 1 SV = 3 | | | | | | | |
| SRC8_HUMAN | Src substrate cortactin - Homo | AKT[181.014]QTPPVS[166.9984] | 1.05 | 0.9040 | 2.36 | 0.0480 | 2.48 | 0.0330 |
| | sapiens (Human) | PAPQPTEER | | | | | | |
| SRC8_HUMAN | Src substrate cortactin - Homo | AKTQT[181.014]PPVSPAPQPT | 2.82 | 0.0170 | −1.87 | 0.3170 | 1.51 | 0.4040 |
| | sapiens (Human) | EER | | | | | | |
| SRC8_HUMAN | Src substrate cortactin - Homo | ASAGHAVS[166.9984]IAQDD | −2.60 | 0.0640 | 1.47 | 0.6810 | −1.77 | 0.4520 |
| | sapiens (Human) | AGADDWETDPDFVNDVSEK | | | | | | |
| SRC8_HUMAN | Src substrate cortactin - Homo | HC[160.0307]S[166.9984]QV | −5.46 | 0.6820 | 4.31 | 0.6690 | −1.27 | 0.8690 |
| | sapiens (Human) | DSVR | | | | | | |
| SRC8_HUMAN | Src substrate cortactin - Homo | LPS[166.9984]SPVYEDAASFK | 1.44 | 0.7990 | 1.11 | 0.8780 | 1.60 | 0.5530 |
| | sapiens (Human) | | | | | | | |
| SRC8_HUMAN | Src substrate cortactin - Homo | LPSS[166.9984]PVYEDAASFK | −1.14 | 0.7200 | 1.58 | 0.4450 | 1.39 | 0.5800 |
| | sapiens (Human) | | | | | | | |
| SRC8_HUMAN | Src substrate cortactin - Homo | T[181.014]QTPPVS[166.9984] | −1.61 | 0.2600 | 1.90 | 0.2820 | 1.18 | 0.8840 |
| | sapiens (Human) | PAPQPTEER | | | | | | |
| SRC8_HUMAN | Src substrate cortactin - Homo | TQT[181.014]PPVS[166.9984] | −1.30 | 0.5330 | 2.30 | 0.0030 | 1.76 | 0.0590 |
| | sapiens (Human) | PAPQPTEER | | | | | | |
| SRC8_HUMAN | Src substrate cortactin - Homo | TQT[181.014]PPVSPAPQPTEER | 1.63 | 0.2930 | −1.08 | 0.9240 | 1.50 | 0.3610 |
| | sapiens (Human) | | | | | | | |
| SRCH_HUMAN | Sarcoplasmic reticulum histidine- | AEVGAPLS[166.9984]PDHS[166.9984] | 1.81 | 0.2230 | −1.65 | 0.3710 | 1.10 | 0.7700 |
| | rich calcium-binding protein | EEEEEEEEGLEEDEPR | | | | | | |
| | OS = Homo sapiens GN = HRC PE = 2 | | | | | | | |
| | SV = 1 | | | | | | | |
| SRCH_HUMAN | Sarcoplasmic reticulum histidine- | AEVGAPLS[166.9984]PDHSEE | −1.16 | 0.5740 | −1.00 | 0.8520 | −1.16 | 0.3890 |
| | rich calcium-binding protein | EEEEEEGLEEDEPR | | | | | | |
| | OS = Homo sapiens GN = HRC PE = 2 | | | | | | | |
| | SV = 1 | | | | | | | |
| SRCH_HUMAN | Sarcoplasmic reticulum histidine- | AEVGAPLSPDHS[166.9984]EE | −1.26 | 0.4420 | 1.09 | 0.9780 | −1.16 | 0.4490 |
| | rich calcium-binding protein | EEEEEEGLEEDEPR | | | | | | |
| | OS = Homo sapiens GN = HRC PE = 2 | | | | | | | |
| | SV = 1 | | | | | | | |
| SRCH_HUMAN | Sarcoplasmic reticulum histidine- | DDSEEEKEKEEDPGS[166.9984] | −1.32 | 0.6670 | −1.02 | 0.9590 | −1.34 | 0.7030 |
| | rich calcium-binding protein | HEEDDESSEQGEK | | | | | | |
| | OS = Homo sapiens GN = HRC PE = 2 | | | | | | | |
| | SV = 1 | | | | | | | |
| SRCH_HUMAN | Sarcoplasmic reticulum histidine- | DEEEDEDVS[166.9984]TER | −1.30 | 0.7960 | −1.11 | 0.9190 | −1.44 | 0.6630 |
| | rich calcium-binding protein | | | | | | | |
| | OS = Homo sapiens GN = HRC PE = 2 | | | | | | | |
| | SV = 1 | | | | | | | |
| SRCH_HUMAN | Sarcoplasmic reticulum histidine- | EEAGGASS[166.9984]EEESGE | −1.37 | 0.4430 | −1.20 | 0.8120 | −1.64 | 0.2200 |
| | rich calcium-binding protein | DTGPQDAQEYGNYQPGSLC[160.0307] | | | | | | |
| | OS = Homo sapiens GN = HRC PE = 2 | GYC[160.0307]SFC[160.0307] | | | | | | |
| | SV = 1 | NR | | | | | | |
| SRCH_HUMAN | Sarcoplasmic reticulum histidine- | EEDEEVS[166.9984]AELGHQ | −2.20 | 0.0700 | 1.20 | 0.9980 | −1.84 | 0.1130 |
| | rich calcium-binding protein | APSHR | | | | | | |
| | OS = Homo sapiens GN = HRC PE = 2 | | | | | | | |
| | SV = 1 | | | | | | | |
| SRCH_HUMAN | Sarcoplasmic reticulum histidine- | EKEEDPGS[166.9984]HEEDDE | −3.18 | 0.0040 | 1.65 | 0.4850 | −1.92 | 0.3070 |
| | rich calcium-binding protein | SSEQGEK | | | | | | |
| | OS = Homo sapiens GN = HRC PE = 2 | | | | | | | |
| | SV = 1 | | | | | | | |
| SRCH_HUMAN | Sarcoplasmic reticulum histidine- | GHDGEDDEGEEEEEEEEEEEEA | −2.57 | 0.0280 | −1.36 | 0.6690 | −3.50 | 0.0040 |
| | rich calcium-binding protein | S[166.9984]TEYGHQAHR | | | | | | |
| | OS = Homo sapiens GN = HRC PE = 2 | | | | | | | |
| | SV = 1 | | | | | | | |
| SRCH_HUMAN | Sarcoplasmic reticulum histidine- | GHGS[166.9984]EEDEDVSDG | −1.73 | 0.5800 | 1.09 | 0.6290 | −1.58 | 0.3670 |
| | rich calcium-binding protein | HHHHGPSHR | | | | | | |
| | OS = Homo sapiens GN = HRC PE = 2 | | | | | | | |
| | SV = 1 | | | | | | | |
| SRCH_HUMAN | Sarcoplasmic reticulum histidine- | GHGSEDT[181.014]EDSAEHR | −1.66 | 0.9990 | −2.62 | 0.0000 | −4.36 | 0.0000 |
| | rich calcium-binding protein | | | | | | | |
| | OS = Homo sapiens GN = HRC PE = 2 | | | | | | | |
| | SV = 1 | | | | | | | |
| SRCH_HUMAN | Sarcoplasmic reticulum histidine- | GHGSEDTEDS[166.9984]AEHR | −1.28 | 0.7460 | −2.61 | 0.6460 | −3.34 | 0.1870 |
| | rich calcium-binding protein | | | | | | | |
| | OS = Homo sapiens GN = HRC PE = 2 | | | | | | | |
| | SV = 1 | | | | | | | |
| SRCH_HUMAN | Sarcoplasmic reticulum histidine- | GHKS[166.9984]DEEDFQDEYK | 1.23 | 0.7950 | −1.04 | 0.8430 | 1.18 | 0.9510 |
| | rich calcium-binding protein | | | | | | | |
| | OS = Homo sapiens GN = HRC PE = 2 | | | | | | | |
| | SV = 1 | | | | | | | |
| SRCH_HUMAN | Sarcoplasmic reticulum histidine- | HQGHEEDDDDDDDDDDDDD | −1.88 | 0.1740 | 1.17 | 0.8530 | −1.61 | 0.3250 |
| | rich calcium-binding protein | DDDVS[166.9984]IEYR | | | | | | |
| | OS = Homo sapiens GN = HRC PE = 2 | | | | | | | |
| | SV = 1 | | | | | | | |
| SRCH_HUMAN | Sarcoplasmic reticulum histidine- | HQGHRDEEEDEDVS[166.9984] | −1.07 | 0.9980 | −1.16 | 0.4660 | −1.25 | 0.6910 |
| | rich calcium-binding protein | TER | | | | | | |
| | OS = Homo sapiens GN = HRC PE = 2 | | | | | | | |
| | SV = 1 | | | | | | | |
| SRCH_HUMAN | Sarcoplasmic reticulum histidine- | HRS[166.9984]HEEDDNDDDD | −1.04 | 0.9400 | 1.68 | 0.7560 | 1.63 | 0.7210 |
| | rich calcium-binding protein | VS[166.9984]TEYGHQAHR | | | | | | |
| | OS = Homo sapiens GN = HRC PE = 2 | | | | | | | |
| | SV = 1 | | | | | | | |
| SRCH_HUMAN | Sarcoplasmic reticulum histidine- | HRS[166.9984]HEEDDNDDDD | −1.92 | 0.4490 | 1.17 | 0.9720 | −1.64 | 0.4280 |
| | rich calcium-binding protein | VSTEYGHQAHR | | | | | | |
| | OS = Homo sapiens GN = HRC PE = 2 | | | | | | | |
| | SV = 1 | | | | | | | |
| SRCH_HUMAN | Sarcoplasmic reticulum histidine- | SHEEDDNDDDDVS[166.9984] | −1.11 | 0.8120 | −1.11 | 0.6430 | −1.24 | 0.5840 |
| | rich calcium-binding protein | TEYGHQAHR | | | | | | |
| | OS = Homo sapiens GN = HRC PE = 2 | | | | | | | |
| | SV = 1 | | | | | | | |
| SRCH_HUMAN | Sarcoplasmic reticulum histidine- | VGDEGVS[166.9984]GEEVFA | −1.31 | 0.6320 | −2.26 | 0.0070 | −2.97 | 0.0030 |
| | rich calcium-binding protein | EHGGQAR | | | | | | |
| | OS = Homo sapiens GN = HRC PE = 2 | | | | | | | |
| | SV = 1 | | | | | | | |
| SRCH_HUMAN | Sarcoplasmic reticulum histidine- | VPREEDEEVS[166.9984]AELG | 1.18 | 0.1720 | −1.34 | 0.2200 | −1.13 | 0.4850 |
| | rich calcium-binding protein | HQAPSHR | | | | | | |
| | OS = Homo sapiens GN = HRC PE = 2 | | | | | | | |
| | SV = 1 | | | | | | | |
| SRRM1_HUMAN | Serine/arginine repetitive matrix | AAS[166.9984]PS[166.9984]P | 1.04 | 0.9880 | −1.47 | 0.6650 | −1.42 | 0.5840 |
| | protein 1 - Homo sapiens (Human) | QSVR | | | | | | |
| SRRM1_HUMAN | Serine/arginine repetitive matrix | AAS[166.9984]PSPQSVR | 1.09 | 0.9840 | −1.74 | 0.5180 | −1.59 | 0.2090 |
| | protein 1 - Homo sapiens (Human) | | | | | | | |
| SRRM1_HUMAN | Serine/arginine repetitive matrix | APQTSSS[166.9984]PPPVR | 1.31 | 0.8320 | −1.46 | 0.6450 | −1.12 | 0.6180 |
| | protein 1 - Homo sapiens (Human) | | | | | | | |
| SRRM1_HUMAN | Serine/arginine repetitive matrix | HRPS[166.9984]PPAT[181.014] | 1.05 | 0.9980 | −1.30 | 0.5630 | −1.24 | 0.4170 |
| | protein 1 - Homo sapiens (Human) | PPPK | | | | | | |
| SRRM1_HUMAN | Serine/arginine repetitive matrix | KEKT[181.014]PELPEPSVK | 1.23 | 0.8560 | −1.58 | 0.4890 | −1.29 | 0.7030 |
| | protein 1 - Homo sapiens (Human) | | | | | | | |
| SRRM1_HUMAN | Serine/arginine repetitive matrix | KET[181.014]ESEAEDNLDDLEK | 1.06 | 0.9370 | −1.31 | 0.5350 | −1.23 | 0.5550 |
| | protein 1 - Homo sapiens (Human) | | | | | | | |
| SRRM1_HUMAN | Serine/arginine repetitive matrix | KETES[166.9984]EAEDNLDDL | 1.10 | 0.8650 | −1.11 | 0.7760 | −1.01 | 0.9040 |
| | protein 1 - Homo sapiens (Human) | EK | | | | | | |
| SRRM1_HUMAN | Serine/arginine repetitive matrix | KPPAPPS[166.9984]PVQSQS | −1.06 | 0.9390 | −1.42 | 0.5820 | −1.50 | 0.3360 |
| | protein 1 - Homo sapiens (Human) | [166.9984]PSTNWSPAVPVK | | | | | | |
| SRRM1_HUMAN | Serine/arginine repetitive matrix | KPPAPPS[166.9984]PVQSQS | −1.30 | 0.5850 | −1.17 | 0.8940 | −1.52 | 0.2770 |
| | protein 1 - Homo sapiens (Human) | [166.9984]PSTNWSPAVPVKK | | | | | | |
| SRRM1_HUMAN | Serine/arginine repetitive matrix | KS[166.9984]RVSVS[166.9984] | 1.42 | 0.9200 | −1.87 | 0.6410 | −1.31 | 0.5440 |
| | protein 1 - Homo sapiens (Human) | PGR | | | | | | |
| SRRM1_HUMAN | Serine/arginine repetitive matrix | KVELS[166.9984]ES[166.9984] | 1.11 | 0.9200 | −1.14 | 0.8530 | −1.02 | 0.9430 |
| | protein 1 - Homo sapiens (Human) | EEDKGGK | | | | | | |
| SRRM1_HUMAN | Serine/arginine repetitive matrix | KVELS[166.9984]ESEEDKGGK | 1.25 | 0.7540 | −1.44 | 0.5630 | −1.15 | 0.7780 |
| | protein 1 - Homo sapiens (Human) | | | | | | | |
| SRRM1_HUMAN | Serine/arginine repetitive matrix | RES[166.9984]PS[166.9984]P | 1.07 | 0.7950 | −1.35 | 0.8080 | −1.26 | 0.2460 |
| | protein 1 - Homo sapiens (Human) | APKPR | | | | | | |
| SRRM1_HUMAN | Serine/arginine repetitive matrix | RES[166.9984]PSPAPKPR | 1.60 | 0.5850 | −1.66 | 0.4540 | −1.03 | 0.9130 |
| | protein 1 - Homo sapiens (Human) | | | | | | | |
| SRRM1_HUMAN | Serine/arginine repetitive matrix | RESPS[166.9984]PAPKPR | 2.10 | 0.4550 | −2.39 | 0.2720 | −1.13 | 0.9110 |
| | protein 1 - Homo sapiens (Human) | | | | | | | |
| SRRM1_HUMAN | Serine/arginine repetitive matrix | RLS[166.9984]PSAS[166.9984] | −1.19 | 0.6180 | −1.15 | 0.8520 | −1.37 | 0.3810 |
| | protein 1 - Homo sapiens (Human) | PPR | | | | | | |
| SRRM1_HUMAN | Serine/arginine repetitive matrix | RQS[166.9984]PSPST[181.014] | −1.06 | 0.8900 | −1.22 | 0.8180 | −1.29 | 0.6150 |
| | protein 1 - Homo sapiens (Human) | RPIR | | | | | | |
| SRRM1_HUMAN | Serine/arginine repetitive matrix | RRAS[166.9984]PS[166.9984] | 1.19 | 0.9200 | −3.03 | 0.0000 | −2.54 | 0.0001 |
| | protein 1 - Homo sapiens (Human) | PPPK | | | | | | |
| SRRM1_HUMAN | Serine/arginine repetitive matrix | RRS[166.9984]PS[166.9984]P | 1.16 | 0.9680 | −1.15 | 0.9150 | 1.01 | 0.9070 |
| | protein 1 - Homo sapiens (Human) | APPPR | | | | | | |
| SRRM1_HUMAN | Serine/arginine repetitive matrix | RRS[166.9984]PS[166.9984]P | 1.13 | 0.9650 | −1.16 | 0.9240 | −1.03 | 0.8240 |
| | protein 1 - Homo sapiens (Human) | PPTR | | | | | | |
| SRRM1_HUMAN | Serine/arginine repetitive matrix | RRT[181.014]AS[166.9984]PP | 1.39 | 0.8690 | −1.53 | 0.7220 | −1.10 | 0.7850 |
| | protein 1 - Homo sapiens (Human) | PPPK | | | | | | |
| SRRM1_HUMAN | Serine/arginine repetitive matrix | RRT[181.014]PS[166.9984]PP | 1.28 | 0.7050 | −1.24 | 0.7450 | 1.04 | 0.9160 |
| | protein 1 - Homo sapiens (Human) | PR | | | | | | |
| SRRM1_HUMAN | Serine/arginine repetitive matrix | RRT[181.014]PT[181.014]PPPR | 1.20 | 0.9360 | −1.24 | 0.8230 | −1.03 | 0.8400 |
| | protein 1 - Homo sapiens (Human) | | | | | | | |
| SRRM1_HUMAN | Serine/arginine repetitive matrix | RYS[166.9984]PS[166.9984]P | −1.02 | 0.9190 | −1.35 | 0.8300 | −1.38 | 0.6750 |
| | protein 1 - Homo sapiens (Human) | PPKR | | | | | | |
| SRRM1_HUMAN | Serine/arginine repetitive matrix | S[166.9984]PS[166.9984]PAP | −1.12 | 0.9610 | 1.00 | 0.9410 | −1.12 | 0.9100 |
| | protein 1 - Homo sapiens (Human) | PPR | | | | | | |
| SRRM1_HUMAN | Serine/arginine repetitive matrix | SRVS[166.9984]VS[166.9984] | 1.19 | 0.9200 | −1.49 | 0.4970 | −1.25 | 0.3560 |
| | protein 1 - Homo sapiens (Human) | PGR | | | | | | |
| SRRM1_HUMAN | Serine/arginine repetitive matrix | TAS[166.9984]PPPPPK | 1.46 | 0.6990 | −1.57 | 0.6360 | −1.08 | 0.8920 |
| | protein 1 - Homo sapiens (Human) | | | | | | | |
| SRRM1_HUMAN | Serine/arginine repetitive matrix | VPKPEPIPEPKEPS[166.9984]P | 1.54 | 0.6860 | −1.93 | 0.3300 | −1.25 | 0.4920 |
| | protein 1 - Homo sapiens (Human) | EK | | | | | | |
| SRRM2_HUMAN | Serine/arginine repetitive matrix | AQT[181.014]PPGPSLSGSK | −1.55 | 0.5430 | 1.95 | 0.3840 | 1.26 | 0.8880 |
| | protein 2 - Homo sapiens (Human) | | | | | | | |
| SRRM2_HUMAN | Serine/arginine repetitive matrix | AQT[181.014]PPGPSLSGSKS[ | −1.21 | 0.6860 | 1.06 | 0.9600 | −1.14 | 0.6910 |
| | protein 2 - Homo sapiens (Human) | 166.9984]PC[160.0307]PQEK | | | | | | |
| SRRM2_HUMAN | Serine/arginine repetitive matrix | AQTPPGPSLS[166.9984]GSKS | −1.21 | 0.6040 | 1.25 | 0.7550 | 1.03 | 0.9510 |
| | protein 2 - Homo sapiens (Human) | PC[160.0307]PQEK | | | | | | |
| SRRM2_HUMAN | Serine/arginine repetitive matrix | GEFSAS[166.9984]PM[147.0354] | 2.24 | 0.5190 | −2.03 | 0.5750 | 1.10 | 0.9800 |
| | protein 2 - Homo sapiens (Human) | LK | | | | | | |
| SRRM2_HUMAN | Serine/arginine repetitive matrix | GEGDAPFSEPGTTSTQRPS[166.9984] | −1.00 | 0.9900 | −1.10 | 0.7240 | −1.11 | 0.6950 |
| | protein 2 - Homo sapiens (Human) | SPETATK | | | | | | |
| SRRM2_HUMAN | Serine/arginine repetitive matrix | HAS[166.9984]S[166.9984]S | 1.11 | 0.9990 | −2.07 | 0.2900 | −1.88 | 0.1030 |
| | protein 2 - Homo sapiens (Human) | [166.9984]PES[166.9984]PKP | | | | | | |
| | | APAPGSHR | | | | | | |
| SRRM2_HUMAN | Serine/arginine repetitive matrix | HGGS[166.9984]PQPLATTPLS | 1.02 | 0.9480 | −1.37 | 0.4930 | −1.34 | 0.4980 |
| | protein 2 - Homo sapiens (Human) | QEPVNPPSEASPT[181.014]R | | | | | | |
| SRRM2_HUMAN | Serine/arginine repetitive matrix | NHS[166.9984]GSRT[181.014] | 1.04 | 0.9840 | 1.16 | 0.8950 | 1.21 | 0.8770 |
| | protein 2 - Homo sapiens (Human) | PPVALNSSR | | | | | | |
| SRRM2_HUMAN | Serine/arginine repetitive matrix | NHSGS[166.9984]RT[181.014] | −1.03 | 0.9640 | 1.14 | 0.9160 | 1.11 | 0.9510 |
| | protein 2 - Homo sapiens (Human) | PPVALNSSR | | | | | | |
| SRRM2_HUMAN | Serine/arginine repetitive matrix | RGEGDAPFSEPGTTSTQRPS[166.9984] | 1.17 | 0.8070 | −1.10 | 0.7520 | 1.06 | 0.8700 |
| | protein 2 - Homo sapiens (Human) | SPETATK | | | | | | |
| SRRM2_HUMAN | Serine/arginine repetitive matrix | RGEGDAPFSEPGTTSTQRPSS[166.9984] | 1.07 | 0.9350 | −1.20 | 0.6280 | −1.13 | 0.7030 |
| | protein 2 - Homo sapiens (Human) | PETATK | | | | | | |
| SRRM2_HUMAN | Serine/arginine repetitive matrix | RPS[166.9984]PQPSPR | 1.22 | 0.8780 | −1.62 | 0.6400 | −1.33 | 0.7030 |
| | protein 2 - Homo sapiens (Human) | | | | | | | |
| SRRM2_HUMAN | Serine/arginine repetitive matrix | RRPS[166.9984]PQPS[166.9984] | 1.19 | 0.6380 | 1.35 | 0.8580 | 1.60 | 0.6820 |
| | protein 2 - Homo sapiens (Human) | PR | | | | | | |
| SRRM2_HUMAN | Serine/arginine repetitive matrix | RRPS[166.9984]PQPSPR | 1.43 | 0.6330 | −1.12 | 0.8120 | 1.28 | 0.9510 |
| | protein 2 - Homo sapiens (Human) | | | | | | | |
| SRRM2_HUMAN | Serine/arginine repetitive matrix | RVPS[166.9984]PTPAPK | 2.53 | 0.2500 | −1.79 | 0.5050 | 1.41 | 0.7270 |
| | protein 2 - Homo sapiens (Human) | | | | | | | |
| SRRM2_HUMAN | Serine/arginine repetitive matrix | S[166.9984]RS[166.9984]PS | 1.24 | 0.9040 | −1.17 | 0.9210 | 1.06 | 0.9720 |
| | protein 2 - Homo sapiens (Human) | [166.9984]SPELNNK | | | | | | |
| SRRM2_HUMAN | Serine/arginine repetitive matrix | SATRPS[166.9984]PS[166.9984] | 1.37 | 0.0000 | −1.32 | 0.0000 | 1.03 | 0.9210 |
| | protein 2 - Homo sapiens (Human) | PER | | | | | | |
| SRRM2_HUMAN | Serine/arginine repetitive matrix | SC[160.0307]FESS[166.9984] | 1.07 | 0.8930 | −1.06 | 0.8030 | 1.01 | 0.9040 |
| | protein 2 - Homo sapiens (Human) | PDPELK | | | | | | |
| SRRM2_HUMAN | Serine/arginine repetitive matrix | SRT[181.014]PPSAPSQSR | −1.17 | 0.5700 | 1.13 | 0.9350 | −1.04 | 0.6930 |
| | protein 2 - Homo sapiens (Human) | | | | | | | |
| SRRM2_HUMAN | Serine/arginine repetitive matrix | SSS[166.9984]PVTELASR | 1.25 | 0.6860 | −1.22 | 0.6660 | 1.03 | 0.8960 |
| | protein 2 - Homo sapiens (Human) | | | | | | | |
| SRRM2_HUMAN | Serine/arginine repetitive matrix | SST[181.014]PPGESYFGVSSL | 1.33 | 0.7790 | −1.60 | 0.5910 | −1.20 | 0.8660 |
| | protein 2 - Homo sapiens (Human) | QLK | | | | | | |
| SRRM2_HUMAN | Serine/arginine repetitive matrix | SSTPPGESY[243.0297]FGVSSL | 1.12 | 0.9070 | −1.61 | 0.4460 | −1.44 | 0.6150 |
| | protein 2 - Homo sapiens (Human) | QLK | | | | | | |
| SRRM2_HUMAN | Serine/arginine repetitive matrix | THTTALAGRS[166.9984]PS[166.9984] | 1.06 | 0.9630 | −1.07 | 0.8520 | −1.01 | 0.8770 |
| | protein 2 - Homo sapiens (Human) | PASGR | | | | | | |
| SRRM2_HUMAN | Serine/arginine repetitive matrix | THTTALAGRS[166.9984]PSPA | 1.19 | 0.8730 | −1.19 | 0.7470 | 1.00 | 0.9020 |
| | protein 2 - Homo sapiens (Human) | S[166.9984]GR | | | | | | |
| SRRM2_HUMAN | Serine/arginine repetitive matrix | THTTALAGRS[166.9984]PSPA | −1.35 | 0.5910 | −1.17 | 0.8580 | −1.59 | 0.3660 |
| | protein 2 - Homo sapiens (Human) | S[166.9984]GRR | | | | | | |
| SRRM2_HUMAN | Serine/arginine repetitive matrix | VSGRT[181.014]SPPLLDR | 1.97 | 0.4720 | −1.95 | 0.4450 | 1.01 | 0.9120 |
| | protein 2 - Homo sapiens (Human) | | | | | | | |
| STA13_HUMAN | StAR-related lipid transfer protein | HKGSGRT[181.014]GGLVISGP | 1.00 | 0.9060 | −2.30 | 0.0740 | −2.30 | 0.3230 |
| | 13 OS = Homo sapiens GN = STARD13 | MLQQEPES[166.9984]FK | | | | | | |
| | PE = 1 SV = 2 | | | | | | | |
| STMN1_HUMAN | Stathmin - Homo sapiens (Human) | RAS[166.9984]GQAFELILSPR | 1.80 | 0.5740 | −1.14 | 0.9330 | 1.58 | 0.5960 |
| STUB1_HUMAN | STIP1 homology and U box- | LGAGGGS[166.9984]PEKSPSA | 1.20 | 0.9040 | −1.45 | 0.6560 | −1.21 | 0.7280 |
| | containing protein 1 OS = Homo | QELK | | | | | | |
| | sapiens GN = STUB1 PE = 1 SV = 2 | | | | | | | |
| SUV41_HUMAN | Histone-lysine N- | QSM[147.0354]SRIPASS[166.9984] | −1.06 | 0.8970 | 1.18 | 0.8790 | 1.11 | 0.9830 |
| | methyltransferase SUV420H1 | NS[166.9984]T[181.014] | | | | | | |
| | OS = Homo sapiens GN = SUV420H1 | S[166.9984]SKLTHINNSR | | | | | | |
| | PE = 1 SV = 2 | | | | | | | |
| SYCP1_HUMAN | Synaptonemal complex protein 1 | SVST[181.014]QKALEEDLQIAT | 1.44 | 0.3930 | −1.61 | 0.1790 | −1.11 | 0.9020 |
| | OS = Homo sapiens GN = SYCP1 PE = 1 | [181.014]K | | | | | | |
| | SV = 2 | | | | | | | |
| SYG_HUMAN | Glycyl-tRNA synthetase - Homo | T[181.014]FFSFPAVVAPFKC[160.0307] | −1.23 | 0.6110 | −1.44 | 0.5480 | −1.77 | 0.1370 |
| | sapiens (Human) | SVLPLSQNQEFM[147.0354] | | | | | | |
| | | PFVK | | | | | | |
| SYNP2_HUMAN | Synaptopodin-2 OS = Homo sapiens | AQS[166.9984]PTPS[166.9984] | 1.90 | 0.3190 | −1.57 | 0.5630 | 1.21 | 0.8250 |
| | GN = SYNPO2 PE = 1 SV = 2 | LPASWK | | | | | | |
| SYNP2_HUMAN | Synaptopodin-2 OS = Homo sapiens | AQS[166.9984]PTPSLPASWK | 1.86 | 0.3490 | −1.30 | 0.7930 | 1.43 | 0.6750 |
| | GN = SYNPO2 PE = 1 SV = 2 | | | | | | | |
| SYNPO_HUMAN | Synaptopodin OS = Homo sapiens | AAS[166.9984]PAKPSSLDLVP | 2.92 | 0.0520 | −1.09 | 0.9200 | 2.68 | 0.0240 |
| | GN = SYN PO PE = 1 SV = 2 | NLPK | | | | | | |
| SYTS_HUMAN | Synaptotagmin-5 OS = Homo | VY[243.0297]LLPDKR | 3.66 | 0.1210 | −1.58 | 0.7560 | 2.32 | 0.3610 |
| | sapiens GN = SYTS PE = 2 SV = 2 | | | | | | | |
| TAU_HUMAN | Microtubule-associated protein | SPVVSGDTS[166.9984]PR | −1.05 | 0.9200 | −1.30 | 0.8520 | −1.37 | 0.7270 |
| | tau - Homo sapiens (Human) | | | | | | | |
| TAU_HUMAN | Microtubule-associated protein | TDHGAEIVYKS[166.9984]PVV | −1.00 | 0.9280 | −1.01 | 0.9300 | −1.01 | 0.9960 |
| | tau - Homo sapiens (Human) | S[166.9984]GDTS[166.9984] | | | | | | |
| | | PR | | | | | | |
| TCAL3_HUMAN | Transcription elongation factor A | GTDDS[166.9984]PKDSQEDL | 1.21 | 0.7820 | 1.18 | 0.9410 | 1.44 | 0.7170 |
| | protein-like 3 OS = Homo sapiens | QER | | | | | | |
| | GN = TCEAL3 PE = 1 SV = 1 | | | | | | | |
| TCAL3_HUMAN | Transcription elongation factor A | NEGNLENEGKPEDEVEPDDEG | 1.14 | 0.7260 | 1.00 | 0.8260 | 1.14 | 0.9830 |
| | protein-like 3 OS = Homo sapiens | KS[166.9984]DEEEKPDVEGK | | | | | | |
| | GN = TCEAL3 PE = 1 SV = 1 | | | | | | | |
| TCEA1_HUMAN | Transcription elongation factor A | KKEPAITSQNS[166.9984]PEAR | 1.60 | 0.4400 | 1.35 | 0.3480 | 2.16 | 0.0003 |
| | protein 1 OS = Homo sapiens | | | | | | | |
| | GN = TCEA1 PE = 1 SV = 2 | | | | | | | |
| TCEA3_HUMAN | Transcription elongation factor A | GLEC[160.0307]SDWKPEAGL | 1.28 | 0.8610 | −1.42 | 0.7580 | −1.11 | 0.9080 |
| | protein 3 OS = Homo sapiens | S[166.9984]PPR | | | | | | |
| | GN = TCEA3 PE = 2 SV = 2 | | | | | | | |
| TCOF_HUMAN | Treacle protein OS = Homo sapiens | KLS[166.9984]GDQPAAR | −1.44 | 0.9200 | −1.61 | 0.4450 | −2.33 | 0.0450 |
| | GN = TCOF1 PE = 1 SV = 2 | | | | | | | |
| TCOF_HUMAN | Treacle protein OS = Homo sapiens | SLGNILQAKPT[181.014]SSPAK | 1.44 | 0.6460 | −1.72 | 0.4690 | −1.20 | 0.7260 |
| | GN = TCOF1 PE = 1 SV = 2 | | | | | | | |
| TCOF_HUMAN | Treacle protein OS = Homo sapiens | TSQVGAASAPAKES[166.9984] | 1.32 | 0.8140 | −1.75 | 0.4920 | −1.32 | 0.5440 |
| | GN = TCOF1 PE = 1 SV = 2 | PR | | | | | | |
| TEBP_HUMAN | Prostaglandin E synthase 3 - Homo | DWEDDS[166.9984]DEDMSN | −2.65 | 0.3560 | 1.78 | 0.7970 | −1.49 | 0.7890 |
| | sapiens (Human) | FDR | | | | | | |
| TEBP_HUMAN | Prostaglandin E synthase 3 - Homo | LNWLSVDFNNWKDWEDDS[166.9984] | 1.60 | 0.9200 | −2.24 | 0.7520 | −1.41 | 0.8130 |
| | sapiens (Human) | DEDM[147.0354]SN | | | | | | |
| | | FDR | | | | | | |
| TEBP_HUMAN | Prostaglandin E synthase 3 - Homo | LNWLSVDFNNWKDWEDDS[166.9984] | −2.56 | 0.1450 | 1.60 | 0.4970 | −1.60 | 0.6860 |
| | sapiens (Human) | DEDMSNFDR | | | | | | |
| TELT_HUMAN | Telethonin OS = Homo sapiens | EEREDT[181.014]PIQLQELLAL | 1.26 | 0.6920 | 1.07 | 0.9310 | 1.34 | 0.5860 |
| | GN = TCAP PE = 1 SV = 1 | ETALGGQC[160.0307]VDR | | | | | | |
| TELT_HUMAN | Telethonin OS = Homo sapiens | SMS[166.9984]QEAQRG | −1.36 | 0.9200 | −1.19 | 0.9590 | −1.62 | 0.8940 |
| | GN = TCAP PE = 1 SV = 1 | | | | | | | |
| TENS1_HUMAN | Tensin-1 OS = Homo sapiens | AQFSVAGVHTVPGS[166.9984] | 3.06 | 0.0010 | −1.29 | 0.6310 | 2.37 | 0.0550 |
| | GN = TNS1 PE = 1 SV = 2 | PQAR | | | | | | |
| TENS1_HUMAN | Tensin-1 OS = Homo sapiens | VATTPGS[166.9984]PSLGR | 3.24 | 0.0070 | −1.46 | 0.6650 | 2.21 | 0.2410 |
| | GN = TNS1 PE = 1 SV = 2 | | | | | | | |
| TGON2_HUMAN | Trans-Golgi network integral | DSPSKS[166.9984]SAEAQTPE | 1.14 | 0.9040 | −1.40 | 0.7060 | −1.23 | 0.8800 |
| | membrane protein 2 OS = Homo | DTPNK | | | | | | |
| | sapiens GN = TGOLN2 PE = 1 SV = 2 | | | | | | | |
| TGON2_HUMAN | Trans-Golgi network integral | DSPSKSS[166.9984]AEAQTPE | 1.18 | 0.9060 | −1.32 | 0.7640 | −1.12 | 0.9090 |
| | membrane protein 2 OS = Homo | DTPNK | | | | | | |
| | sapiens GN = TGOLN2 PE = 1 SV = 2 | | | | | | | |
| THUM1_HUMAN | THUMP domain-containing protein | FTDKDQQPS[166.9984]GS[166.9984] | 1.28 | 0.3500 | −1.34 | 0.3680 | −1.05 | 0.7340 |
| | 1 OS = Homo sapiens | EGEDDDAEAALKK | | | | | | |
| | GN = THUMPD1 PE = 1 SV = 2 | | | | | | | |
| TIAM2_HUMAN | T-lymphoma invasion and | QDSKS[166.9984]TSPGK | −1.05 | 0.7110 | −1.42 | 0.5240 | −1.49 | 0.0340 |
| | metastasis-inducing protein 2 | | | | | | | |
| | OS = Homo sapiens GN = TIAM2 | | | | | | | |
| | PE = 2 SV = 3 | | | | | | | |
| TITIN_HUMAN | Titin - Homo sapiens (Human) | AVS[166.9984]PTETKPTPTEK | 2.56 | 0.0370 | −2.64 | 0.0450 | −1.03 | 0.9640 |
| TITIN_HUMAN | Titin - Homo sapiens (Human) | AVTS[166.9984]PPR | 1.96 | 0.6180 | −1.76 | 0.6540 | 1.11 | 0.9390 |
| TITIN_HUMAN | Titin - Homo sapiens (Human) | IELSPS[166.9984]M[147.0354] | 3.01 | 0.2240 | −2.48 | 0.3840 | 1.21 | 0.9190 |
| | | EAPK | | | | | | |
| TITIN_HUMAN | Titin - Homo sapiens (Human) | RRT[181.014]PS[166.9984]P | 2.03 | 0.0590 | −2.05 | 0.1770 | −1.01 | 0.7990 |
| | | DYDFYYRPR | | | | | | |
| TITIN_HUMAN | Titin - Homo sapiens (Human) | RVKS[166.9984]PEPSHPK | 3.76 | 0.0000 | −2.18 | 0.0001 | 1.72 | 0.6440 |
| TITIN_HUMAN | Titin - Homo sapiens (Human) | S[166.9984]LS[166.9984]PTYI | 3.73 | 0.3570 | −3.57 | 0.4360 | 1.05 | 0.9820 |
| | | ELM[147.0354]RPVSELIR | | | | | | |
| TITIN_HUMAN | Titin - Homo sapiens (Human) | SRPQPAEEYEDDT[181.014]ER | 1.90 | 0.0640 | −1.54 | 0.3330 | 1.24 | 0.6800 |
| | | RSPT[181.014]PER | | | | | | |
| TITIN_HUMAN | Titin - Homo sapiens (Human) | SRPQPAEEYEDDTERRS[166.9984] | 1.60 | 0.2620 | −1.48 | 0.4320 | 1.08 | 0.9180 |
| | | PTPER | | | | | | |
| TITIN_HUMAN | Titin - Homo sapiens (Human) | SRS[166.9984]PTPPSIAAK | 3.10 | 0.0300 | 1.73 | 0.5250 | 5.36 | 0.0001 |
| TITIN_HUMAN | Titin - Homo sapiens (Human) | VKS[166.9984]PEAVKS[166.9984] | 1.88 | 0.0880 | −1.62 | 0.4390 | 1.16 | 0.7650 |
| | | PK | | | | | | |
| TM130_HUMAN | Transmembrane protein 130 | NAT[181.014]QQKDM[147.0354] | −1.12 | 0.9280 | 1.59 | 0.6760 | 1.42 | 0.7520 |
| | OS = Homo sapiens GN = TMEM130 | VEVADFDFSPM[147.0354] | | | | | | |
| | PE = 2 SV = 1 | S[166.9984]DKNPEPPSGVR | | | | | | |
| TNNC1_HUMAN | Troponin C, slow skeletal and | GKS[166.9984]EEELSDLFR | 1.09 | 0.9580 | −1.85 | 0.5430 | −1.70 | 0.6060 |
| | cardiac muscles OS = Homo sapiens | | | | | | | |
| | GN = TNNC1 PE = 1 SV = 1 | | | | | | | |
| TNNI3_HUMAN | Troponin I, cardiac muscle | RRS[166.9984]S[166.9984]NYR | −1.56 | 0.4490 | −1.30 | 0.5490 | −2.02 | 0.1030 |
| | OS = Homo sapiens GN = TNNI3 PE = 1 | | | | | | | |
| | SV = 3 | | | | | | | |
| TNNT2_HUMAN | Troponin T, cardiac muscle | ELWQS[166.9984]IYNLEAEKF | −1.41 | 0.7480 | −1.09 | 0.9980 | −1.54 | 0.6800 |
| | OS = Homo sapiens GN = TNNT2 | DLQEK | | | | | | |
| | PE = 1 SV = 3 | | | | | | | |
| TNR16_HUMAN | Tumor necrosis factor receptor | LHSDSGISVDS[166.9984]QSL | 1.20 | 0.9040 | −1.14 | 0.8030 | 1.05 | 0.9480 |
| | superfamily member 16 OS = Homo | HDQQPHTQTASGQALK | | | | | | |
| | sapiens GN = NGFR PE = 1 SV = 1 | | | | | | | |
| TOIP1_HUMAN | Torsin-1A-interacting protein 1 | VNFSEEGET[181.014]EEDDQ | −1.21 | 0.7200 | 1.28 | 0.6890 | 1.06 | 0.9130 |
| | OS = Homo sapiens GN = TOR1AIP1 | DSSHSSVTTVK | | | | | | |
| | PE = 1 SV = 2 | | | | | | | |
| TOM70_HUMAN | Mitochondrial precursor proteins | AS[166.9984]PAPGSGHPEGP | 3.63 | 0.2930 | −2.15 | 0.6680 | 1.69 | 0.6620 |
| | import receptor - Homo sapiens | GAHLDM[147.0354]NSLDR | | | | | | |
| | (Human) | | | | | | | |
| TPD53_HUMAN | Tumor protein D53 OS = Homo | NSPTFKS[166.9984]FEER | −1.08 | 0.9110 | 1.12 | 0.9590 | 1.04 | 0.9490 |
| | sapiens GN = TPD52L1 PE = 1 SV = 1 | | | | | | | |
| TPD54_HUMAN | Tumor protein D54 - Homo sapiens | NSATFKS[166.9984]FEDR | 2.65 | 0.0020 | −1.24 | 0.6850 | 2.15 | 0.0640 |
| | (Human) | | | | | | | |
| TPIS_HUMAN | Triosephosphate isomerase - | IIYGGS[166.9984]VTGATC[160.0307]K | −1.24 | 0.8240 | −1.29 | 0.7430 | −1.60 | 0.3980 |
| | Homo sapiens (Human) | | | | | | | |
| TPIS_HUMAN | Triosephosphate isomerase - | KQS[166.9984]LGELIGTLNAAK | 2.69 | 0.0490 | −2.17 | 0.2160 | 1.24 | 0.7550 |
| | Homo sapiens (Human) | | | | | | | |
| TPM1_HUMAN | Tropomyosin alpha-1 chain - Homo | KLVIIES[166.9984]DLER | 1.14 | 0.9390 | −1.77 | 0.3730 | −1.55 | 0.2830 |
| | sapiens (Human) | | | | | | | |
| TPM1_HUMAN | Tropomyosin alpha-1 chain - Homo | LVIIES[166.9984]DLERAEER | 1.10 | 0.9200 | −1.65 | 0.4350 | −1.51 | 0.3210 |
| | sapiens (Human) | | | | | | | |
| TPM1_HUMAN | Tropomyosin alpha-1 chain - Homo | S[166.9984]IDDLEDELYAQK | −1.23 | 0.8540 | −1.47 | 0.4790 | −1.81 | 0.2720 |
| | sapiens (Human) | | | | | | | |
| TPM1_HUMAN | Tropomyosin alpha-1 chain | AISEELDHALNDM[147.0354]T | 2.39 | 0.2410 | −2.79 | 0.1420 | −1.16 | 0.8130 |
| | OS = Homo sapiens GN = TPM1 PE = 1 | S[166.9984]I | | | | | | |
| | SV = 2 | | | | | | | |
| TPM1_HUMAN | Tropomyosin alpha-1 chain | AISEELDHALNDMTS[166.9984]I | −1.89 | 0.5710 | 1.05 | 0.6890 | −1.80 | 0.5270 |
| | OS = Homo sapiens GN = TPM1 PE = 1 | | | | | | | |
| | SV = 2 | | | | | | | |
| TPM1_HUMAN | Tropomyosin alpha-1 chain | ATDAEADVAS[166.9984]LNRR | 1.45 | 0.6820 | −2.24 | 0.2710 | −1.54 | 0.5640 |
| | OS = Homo sapiens GN = TPM1 PE = 1 | | | | | | | |
| | SV = 2 | | | | | | | |
| TPM1_HUMAN | Tropomyosin alpha-1 chain | KATDAEADVAS[166.9984]LNR | 1.33 | 0.7830 | −1.97 | 0.3120 | −1.48 | 0.6630 |
| | OS = Homo sapiens GN = TPM1 PE = 1 | | | | | | | |
| | SV = 2 | | | | | | | |
| TPM1_HUMAN | Tropomyosin alpha-1 chain | KATDAEADVAS[166.9984]LNRR | 1.49 | 0.8380 | −2.00 | 0.5880 | −1.34 | 0.7090 |
| | OS = Homo sapiens GN = TPM1 PE = 1 | | | | | | | |
| | SV = 2 | | | | | | | |
| TPM1_HUMAN | Tropomyosin alpha-1 chain | LAT[181.014]ALQK | −1.32 | 0.8750 | −2.27 | 0.3840 | −3.00 | 0.1420 |
| | OS = Homo sapiens GN = TPM1 PE = 1 | | | | | | | |
| | SV = 2 | | | | | | | |
| TPM2_HUMAN | Tropomyosin beta chain - Homo | AISEELDNALNDITS[166.9984]L | 1.12 | 0.7050 | −1.35 | 0.1760 | −1.20 | 0.6930 |
| | sapiens (Human) | | | | | | | |
| TPPP_HUMAN | Tubulin polymerization-promoting | AANRT[181.014]PPKSPGDPSK | 1.05 | 0.0000 | −2.47 | 0.0000 | −2.36 | 0.1420 |
| | protein OS = Homo sapiens | | | | | | | |
| | GN = TPPP PE = 1 SV = 1 | | | | | | | |
| TPPP_HUMAN | Tubulin polymerization-promoting | AISS[166.9984]PTVSR | 3.26 | 0.0300 | −1.71 | 0.4060 | 1.91 | 0.7050 |
| | protein OS = Homo sapiens | | | | | | | |
| | GN = TPPP PE = 1 SV = 1 | | | | | | | |
| TR150_HUMAN | Thyroid hormone receptor- | ASAVSELS[166.9984]PR | 1.86 | 0.1170 | −1.74 | 0.1200 | 1.07 | 0.9040 |
| | associated protein 3 OS = Homo | | | | | | | |
| | sapiens GN = THRAP3 PE = 1 SV = 2 | | | | | | | |
| TR150_HUMAN | Thyroid hormone receptor- | ERS[166.9984]PALKS[166.9984] | −1.96 | 0.0930 | −1.19 | 0.7210 | −2.34 | 0.0190 |
| | associated protein 3 OS = Homo | PLQSVVVR | | | | | | |
| | sapiens GN = THRAP3 PE = 1 SV = 2 | | | | | | | |
| TR150_HUMAN | Thyroid hormone receptor- | GSFS[166.9984]DTGLGDGK | 1.10 | 0.9200 | −1.73 | 0.3830 | −1.57 | 0.3640 |
| | associated protein 3 OS = Homo | | | | | | | |
| | sapiens GN = THRAP3 PE = 1 SV = 2 | | | | | | | |
| TR150_HUMAN | Thyroid hormone receptor- | IDIS[166.9984]PSTFR | 1.41 | 0.4930 | −2.15 | 0.0520 | −1.52 | 0.4050 |
| | associated protein 3 OS = Homo | | | | | | | |
| | sapiens GN = THRAP3 PE = 1 SV = 2 | | | | | | | |
| TR150_HUMAN | Thyroid hormone receptor- | MDS[166.9984]FDEDLARPSG | −3.09 | 0.0390 | 1.37 | 0.8930 | −2.25 | 0.3020 |
| | associated protein 3 OS = Homo | LLAQER | | | | | | |
| | sapiens GN = THRAP3 PE = 1 SV = 2 | | | | | | | |
| TR150_HUMAN | Thyroid hormone receptor- | NKKS[166.9984]PEIHR | 1.35 | 0.9200 | −3.17 | 0.3420 | −2.34 | 0.4830 |
| | associated protein 3 OS = Homo | | | | | | | |
| | sapiens GN = THRAP3 PE = 1 SV = 2 | | | | | | | |
| TR150_HUMAN | Thyroid hormone receptor- | RIDIS[166.9984]PSTFR | 1.61 | 0.5430 | −1.92 | 0.3160 | −1.20 | 0.8160 |
| | associated protein 3 OS = Homo | | | | | | | |
| | sapiens GN = THRAP3 PE = 1 SV = 2 | | | | | | | |
| TR150_HUMAN | Thyroid hormone receptor- | S[166.9984]PPSTGSTYGSSQK | 1.01 | 0.9840 | −1.21 | 0.8540 | −1.20 | 0.7990 |
| | associated protein 3 OS = Homo | | | | | | | |
| | sapiens GN = THRAP3 PE = 1 SV = 2 | | | | | | | |
| TR150_HUMAN | Thyroid hormone receptor- | SPVGKS[166.9984]PPSTGSTY | −1.02 | 0.9980 | −1.29 | 0.5350 | −1.31 | 0.5530 |
| | associated protein 3 OS = Homo | GSSQK | | | | | | |
| | sapiens GN = THRAP3 PE = 1 SV = 2 | | | | | | | |
| TRA2A_HUMAN | Transformer-2 protein homolog | AHT[181.014]PTPGIYM[147.0354] | 2.49 | 0.5330 | −4.52 | 0.1500 | −1.81 | 0.3420 |
| | OS = Homo sapiens GN = TRA2A | GRPTHSGGGGGGGGGG | | | | | | |
| | PE = 1 SV = 1 | GGGGGGR | | | | | | |
| TRA2A_HUMAN | Transformer-2 protein homolog | RRS[166.9984]PS[166.9984]P | 1.17 | 0.7100 | −1.32 | 0.3600 | −1.13 | 0.5140 |
| | OS = Homo sapiens GN = TRA2A | YYSR | | | | | | |
| | PE = 1 SV = 1 | | | | | | | |
| TRA2A_HUMAN | Transformer-2 protein homolog | RRS[166.9984]PSPY[243.0297] | 1.13 | 0.9040 | −1.29 | 0.5850 | −1.14 | 0.6830 |
| | OS = Homo sapiens GN = TRA2A | YSR | | | | | | |
| | PE = 1 SV = 1 | | | | | | | |
| TRA2B_HUMAN | Splicing factor, arginine/serine-rich | RHS[166.9984]HS[166.9984] | −2.20 | 0.9040 | 2.74 | 0.4560 | 1.25 | 0.7270 |
| | 10 OS = Homo sapiens GN = SFRS10 | HS[166.9984]PMSTR | | | | | | |
| | PE = 1 SV = 1 | | | | | | | |
| TRA2B_HUMAN | Splicing factor, arginine/serine-rich | RPHT[181.014]PTPGIYM[147.0354] | 3.27 | 0.6710 | −4.13 | 0.4630 | −1.27 | 0.8070 |
| | 10 OS = Homo sapiens GN = SFRS10 | GRPTYGSSR | | | | | | |
| | PE = 1 SV = 1 | | | | | | | |
| TRAK1_HUMAN | Trafficking kinesin-binding protein | NINQVVKQRS[166.9984]LT[181.014] | 1.12 | 0.7370 | 1.93 | 0.1090 | 2.17 | 0.0660 |
| | 1 OS = Homo sapiens GN = TRAK1 | PS[166.9984]PM[147.0354] | | | | | | |
| | PE = 1 SV = 1 | NIPGS[166.9984]NQSS | | | | | | |
| | | AM[147.0354]NSLLSSC[160.0307] | | | | | | |
| | | VST[181.014]PR | | | | | | |
| TRIP4_HUMAN | Activating signal cointegrator 1 | LERET[181.014]LQK | 1.45 | 0.5790 | −1.14 | 0.7860 | 1.28 | 0.9270 |
| | OS = Homo sapiens GN = TRIP4 PE = 1 | | | | | | | |
| | SV = 4 | | | | | | | |
| TRPC1_HUMAN | Short transient receptor potential | M[147.0354]MAALYPSTDLSG | 1.17 | 0.9350 | −1.13 | 0.8230 | 1.03 | 0.8160 |
| | channel 1 OS = Homo sapiens | ASSSSLPSSPSSS[166.9984]SP | | | | | | |
| | GN = TRPC1 PE = 1 SV = 1 | NEVM[147.0354]ALKDVREVK | | | | | | |
| TTC15_HUMAN | Tetratricopeptide repeat protein | LKDS[166.9984]LR | 1.84 | 0.2930 | −1.46 | 0.5480 | 1.25 | 0.8840 |
| | 15 OS = Homo sapiens GN = TTC15 | | | | | | | |
| | PE = 1 SV = 3 | | | | | | | |
| TTC28_HUMAN | Tetratricopeptide repeat protein | NM[147.0354]S[166.9984]PS | 1.13 | 0.8750 | 1.13 | 0.7920 | 1.27 | 0.5600 |
| | 28 OS = Homo sapiens GN = TTC28 | [166.9984]SGHQSPAGSAPSPA | | | | | | |
| | PE = 2 SV = 3 | LSYSS[166.9984]AGS[166.9984] | | | | | | |
| | | AR | | | | | | |
| TTLL4_HUMAN | Tubulin polyglutamylase TTLL4 | M[147.0354]ASAGT[181.014] | 1.75 | 0.3720 | −1.26 | 0.7800 | 1.39 | 0.6620 |
| | OS = Homo sapiens GN = TTLL4 PE = 1 | QHY[243.0297]S[166.9984]IG | | | | | | |
| | SV = 2 | LRQKNSFK | | | | | | |
| VASP_HUMAN | Vasodilator-stimulated | KVS[166.9984]KQEEASGGPTA | 1.13 | 0.9800 | 1.58 | 0.6770 | 1.79 | 0.6300 |
| | phosphoprotein OS = Homo sapiens | PK | | | | | | |
| | GN = VASP PE = 1 SV = 3 | | | | | | | |
| VDAC1_HUMAN | Voltage-dependent anion-selective | VNNS[166.9984]SLIGLGYTQT | −1.30 | 0.7980 | −1.89 | 0.1600 | −2.44 | 0.0140 |
| | channel protein 1 - Homo sapiens | LKPGIK | | | | | | |
| | (Human) | | | | | | | |
| VDAC2_HUMAN | Voltage-dependent anion-selective | LTFDTTFSPNT[181.014]GK | −1.12 | 0.9840 | −1.15 | 0.8930 | −1.29 | 0.8670 |
| | channel protein 2 - Homo sapiens | | | | | | | |
| | (Human) | | | | | | | |
| VDAC2_HUMAN | Voltage-dependent anion-selective | VNNS[166.9984]SLIGVGYTQT | −1.30 | 0.6780 | −1.87 | 0.1880 | −2.43 | 0.0140 |
| | channel protein 2 - Homo sapiens | LRPGVK | | | | | | |
| | (Human) | | | | | | | |
| VIME_HUMAN | Vimentin - Homo sapiens (Human) | LRS[166.9984]SVPGVR | −1.31 | 0.8830 | 2.31 | 0.6270 | 1.76 | 0.6820 |
| VIME_HUMAN | Vimentin - Homo sapiens (Human) | TYS[166.9984]LGSALRPSTSR | 1.07 | 0.9840 | 4.16 | 0.0210 | 4.45 | 0.0170 |
| VINEX_HUMAN | Vinexin OS = Homo sapiens | LC[160.0307]DDGPQLPTS[166.9984] | 1.55 | 0.6110 | 1.16 | 0.8520 | 1.79 | 0.3040 |
| | GN = SORBS3 PE = 1 SV = 1 | PR | | | | | | |
| WD42A_HUMAN | WD repeat-containing protein 42A | VHDRS[166.9984]EEEEEEEEE | 1.28 | 0.7260 | −1.15 | 0.8440 | 1.12 | 0.9130 |
| | OS = Homo sapiens GN = WDR42A | EEEEQPR | | | | | | |
| | PE = 1 SV = 1 | | | | | | | |
| YETS2_HUMAN | YEATS domain-containing protein 2 | T[181.014]TLFTQAAHGGQAS | −3.54 | 0.0003 | 4.00 | 0.0010 | 1.13 | 0.9040 |
| | OS = Homo sapiens GN = YEATS2 | LM[147.0354]KISDSTLK | | | | | | |
| | PE = 1 SV = 2 | | | | | | | |
| YTDC1_HUMAN | YTH domain-containing protein 1 | GIS[166.9984]PlVFDR | −1.04 | 0.9980 | −1.18 | 0.8750 | −1.23 | 0.8840 |
| | OS = Homo sapiens GN = YTHDC1 | | | | | | | |
| | PE = 1 SV = 3 | | | | | | | |
| YTDC1_HUMAN | YTH domain-containing protein 1 | LSSESHHGGS[166.9984]PIHW | 2.13 | 0.9370 | −3.26 | 0.4970 | −1.53 | 0.3250 |
| | OS = Homo sapiens GN = YTHDC1 | VLPAGM[147.0354]SAK | | | | | | |
| | PE = 1 SV = 3 | | | | | | | |
| ZC3HD_HUMAN | Zinc finger CCCH domain- | SLS[166.9984]PSHLTEDR | 1.90 | 0.2840 | −2.48 | 0.1490 | −1.31 | 0.6960 |
| | containing protein 13 OS = Homo | | | | | | | |
| | sapiens GN = ZC3H13 PE = 1 SV = 1 | | | | | | | |
| ZCH12_HUMAN | Zinc finger CCHC domain- | S[166.9984]LGRSLGPIMASM | −1.79 | 0.9630 | −1.08 | 0.9060 | −1.93 | 0.9020 |
| | containing protein 12 OS = Homo | ADR | | | | | | |
| | sapiens GN = ZCCHC12 PE = 2 SV = 2 | | | | | | | |
| ZCH18_HUMAN | Zinc finger CCCH domain- | LGVSVS[166.9984]PSR | 1.50 | 0.6080 | −1.80 | 0.4270 | −1.20 | 0.5690 |
| | containing protein 18 OS = Homo | | | | | | | |
| | sapiens GN = ZC3H18 PE = 1 SV = 1 | | | | | | | |
| ZEB2_HUMAN | Zinc finger E-box-binding | TGS[166.9984]S[166.9984]P | 2.24 | 0.0410 | −1.10 | 0.8590 | 2.05 | 0.1410 |
| | homeobox 2 OS = Homo sapiens | NS[166.9984]VS[166.9984]SS | | | | | | |
| | GN = ZEB2 PE = 1 SV = 1 | PTNSAITQLRNKLENGKPLSMS | | | | | | |
| | | EQTGLLK | | | | | | |
| ZFP2_HUMAN | Zinc finger protein 2 homolog | C[160.0307]GKSFSQS[166.9984] | −1.28 | 0.7070 | 3.42 | 0.0330 | 2.67 | 0.2930 |
| | OS = Homo sapiens GN = ZFP2 PE = 2 | T[181.014]YLIEHQR | | | | | | |
| | SV = 1 | | | | | | | |
| ZN281_HUMAN | Zinc finger protein 281 OS = Homo | IGS[166.9984]GFLS[166.9984] | −1.23 | 0.8690 | −1.74 | 0.0240 | −2.14 | 0.0980 |
| | sapiens GN = ZNF281 PE = 1 SV = 1 | GGGGTGSSGGSGSGGGGSGG | | | | | | |
| | | GGGGGSSGRR | | | | | | |
| ZN410_HUMAN | Zinc finger protein 410 OS = Homo | QFTT[181.014]AGNLK | −1.01 | 0.9200 | −1.12 | 0.8710 | −1.13 | 0.7650 |
| | sapiens GN = ZNF410 PE = 1 SV = 2 | | | | | | | |
| ZN433_HUMAN | Zinc finger protein 433 OS = Homo | QC[160.0307]GKAFRSASLLQT | −1.18 | 0.1220 | 1.81 | 0.0030 | 1.53 | 0.0930 |
| | sapiens GN = ZNF433 PE = 2 SV = 1 | [181.014]HGR | | | | | | |
| ZN644_HUMAN | Zinc finger protein 644 OS = Homo | RS[166.9984]FLQQDVNK | 1.19 | 0.8320 | −1.15 | 0.8590 | 1.04 | 0.9510 |
| | sapiens GN = ZNF644 PE = 1 SV = 2 | | | | | | | |
| ZNF22_HUMAN | Zinc finger protein 22 OS = Homo | S[166.9984]LDDKPYK | −2.19 | 0.6820 | 1.58 | 0.8270 | −1.39 | 0.9020 |
| | sapiens GN = ZNF22 PE = 1 SV = 3 | | | | | | | |
| ZNF34_HUMAN | Zinc finger protein 34 OS = Homo | DVMLETYGNLVSLGVGPAGPKP | −1.07 | 0.9150 | −1.31 | 0.7430 | −1.40 | 0.6620 |
| | sapiens GN = ZNF34 PE = 2 SV = 2 | GVIS[166.9984]QLER | | | | | | |
| ZNRF2_HUMAN | E3 ubiquitin-protein ligase ZNRF2 | DRPVGGS[166.9984]PGGPR | 1.45 | 0.0000 | 1.07 | 0.0010 | 1.55 | 0.6750 |
| | OS = Homo sapiens GN = ZNRF2 | | | | | | | |
| | PE = 1 SV = 1 | | | | | | | |
| ZP4_HUMAN | Zona pellucida sperm-binding | DKNY[243.0297]GS[166.9984] | −1.29 | 0.2100 | −2.14 | 0.0120 | −2.77 | 0.0000 |
| | protein 4 OS = Homo sapiens | YYGVGDYPVVKLLR | | | | | | |
| | GN = ZP4 PE = 2 SV = 1 | | | | | | | |
| ZRAB2_HUMAN | Zinc finger Ran-binding domain- | ENVEYIEREES[166.9984]DGE | 1.01 | 0.9710 | −1.32 | 0.3160 | −1.30 | 0.2890 |
| | containing protein 2 OS = Homo | YDEFGR | | | | | | |
| | sapiens GN = ZRANB2 PE = 1 SV = 2 | | | | | | | |
| ZRAB2_HUMAN | Zinc finger Ran-binding domain- | EVEDKES[166.9984]EGEEEDE | −1.05 | 0.9190 | −1.08 | 0.8770 | −1.13 | 0.7650 |
| | containing protein 2 OS = Homo | DEDLSK | | | | | | |
| | sapiens GN = ZRANB2 PE = 1 SV = 2 | | | | | | | |
| ZRAB2_HUMAN | Zinc finger Ran-binding domain- | YNLDAS[166.9984]EEEDSNKK | −1.01 | 0.9870 | −1.09 | 0.8520 | −1.11 | 0.8940 |
| | containing protein 2 OS = Homo | | | | | | | |
| | sapiens GN = ZRANB2 PE = 1 SV = 2 | | | | | | | |
| ZSC22_HUMAN | Zinc finger and SCAN domain- | QSDLGESEPS[166.9984]NVTE | 1.50 | 0.6230 | −1.26 | 0.8200 | 1.18 | 0.7550 |
| | containing protein 22 OS = Homo | TLM[147.0354]GGVSLGPAFVK | | | | | | |
| | sapiens GN = ZSCAN22 PE = 1 SV = 2 | | | | | | | |

TABLE 9
EXPRESSION PROFILE OF 68 UNENRICHED OVERLAPPING PROTEINS

| Primary Protein | | ProteinTeller | Peptide |
|---|---|---|---|
| Name | Protein Description | Probability | Count |
| ALBU_HUMAN | Serum albumin OS = Homo sapiens | 1 | 242 |
| | GN = ALB PE = 1 SV = 2 | | |
| ALDOA_HUMAN | Fructose-bisphosphate aldolase A | 1 | 24 |
| | OS = Homo sapiens GN = ALDOA PE = 1 | | |
| | SV = 2 | | |
| ANT3_HUMAN | Antithrombin-III OS = Homo sapiens | 1 | 16 |
| | GN = SERPINC1 PE = 1 SV = 1 | | |
| BASI_HUMAN | Basigin OS = Homo sapiens GN = BSG | 1 | 14 |
| | PE = 1 SV = 2 | | |
| CALD1_HUMAN | Caldesmon OS = Homo sapiens | 1 | 6 |
| | GN = CALD1 PE = 1 SV = 2 | | |
| CAPZB_HUMAN | F-actin-capping protein subunit beta | 1 | 2 |
| | OS = Homo sapiens GN = CAPZB PE = 1 | | |
| | SV = 4 | | |
| CASQ2_HUMAN | Calsequestrin-2 OS = Homo sapiens | 1 | 33 |
| | GN = CASQ2 PE = 1 SV = 2 | | |
| CRIP2_HUMAN | Cysteine-rich protein 2 OS = | 1 | 11 |
| | Homo sapiens GN = CRIP2 PE = 1 SV = 1 | | |
| CRYAB_HUMAN | Alpha-crystallin B chain OS = | 1 | 12 |
| | Homo sapiens GN = CRYAB PE = 1 SV = 2 | | |
| CSPG2_HUMAN | Versican core protein OS = | 1 | 9 |
| | Homo sapiens GN = VCAN PE = 1 SV = 3 | | |
| CSRP3_HUMAN | Cysteine and glycine-rich protein 3 | 1 | 39 |
| | OS = Homo sapiens GN = CSRP3 PE = 1 | | |
| | SV = 1 | | |
| CYC_HUMAN | Cytochrome c OS = Homo sapiens | 1 | 36 |
| | GN = CYCS PE = 1 SV = 2 | | |
| DESM_HUMAN | Desmin OS = Homo sapiens GN = DES | 1 | 33 |
| | PE = 1 SV = 3 | | |
| DSG2_HUMAN | Desmoglein-2 OS = Homo sapiens | 1 | 4 |
| | GN = DSG2 PE = 1 SV = 2 | | |
| EF1B_HUMAN | Elongation factor 1-beta OS = | 0.82 | 1 |
| | Homo sapiens GN = EEF1B2 PE = 1 | | |
| | SV = 3 | | |
| FBLN1_HUMAN | Fibulin-1 OS = Homo sapiens | 0.99 | 2 |
| | GN = FBLN1 PE = 1 SV = 4 | | |
| FETUA_HUMAN | Alpha-2-HS-glycoprotein OS = | 1 | 14 |
| | Homo sapiens GN = AHSG PE = 1 | | |
| | SV = 1 | | |
| FHL2_HUMAN | Four and a half LIM domains | 1 | 40 |
| | protein 2 OS = Homo sapiens GN = | | |
| | FHL2 PE = 1 SV = 3 | | |
| FRIH_HUMAN | Ferritin heavy chain OS = | 1 | 8 |
| | Homo sapiens GN = FTH1 PE = 1 | | |
| | SV = 2 | | |
| G3P_HUMAN | Glyceraldehyde-3-phosphate | 1 | 57 |
| | dehydrogenase OS = Homo sapiens | | |
| | GN = GAPDH PE = 1 SV = 3 | | |
| H12_HUMAN | Histone H1.2 OS = Homo sapiens | 1 | 21 |
| | GN = HIST1H1C PE = 1 SV = 2 | | |
| H31_HUMAN | Histone H3.1 OS = Homo sapiens | 1 | 1 |
| | GN = HIST1H3A PE = 1 SV = 2 | | |
| HNRPD_HUMAN | Heterogeneous nuclear | 1 | 5 |
| | ribonucleoprotein D0 OS = | | |
| | Homo sapiens GN = HNRNPD PE = 1 | | |
| | SV = 1 | | |
| HP1B3_HUMAN | Heterochromatin protein 1-binding | 1 | 4 |
| | protein 3 OS = Homo sapiens | | |
| | GN = HP1BP3 PE = 1 SV = 1 | | |
| HS90A_HUMAN | Heat shock protein HSP 90-alpha | 1 | 16 |
| | OS = Homo sapiens GN = HSP90AA1 | | |
| | PE = 1 SV = 5 | | |
| HS90B_HUMAN | Heat shock protein HSP 90-beta | 1 | 3 |
| | OS = Homo sapiens GN = HSP90AB1 | | |
| | PE = 1 SV = 4 | | |
| HSPB1_HUMAN | Heat shock protein beta-1 OS = | 1 | 24 |
| | Homo sapiens GN = HSPB1 PE = 1 | | |
| | SV = 2 | | |
| HSPB7_HUMAN | Heat shock protein beta-7 OS = | 1 | 13 |
| | Homo sapiens GN = HSPB7 PE = 1 SV = 1 | | |
| ICAL_HUMAN | Calpastatin OS = Homo sapiens | 1 | 20 |
| | GN = CAST PE = 1 SV = 4 | | |
| KAD1_HUMAN | Adenylate kinase isoenzyme 1 | 1 | 19 |
| | OS = Homo sapiens GN = AK1 PE = 1 | | |
| | SV = 3 | | |
| KCRM_HUMAN | Creatine kinase M-type OS = | 1 | 41 |
| | Homo sapiens GN = CKM PE = 1 SV = 2 | | |
| KCRS_HUMAN | Creatine kinase S-type, mitochondrial | 1 | 22 |
| | OS = Homo sapiens GN = CKMT2 PE = 1 | | |
| | SV = 2 | | |
| KNG1_HUMAN | Kininogen-1 OS = Homo sapiens | 1 | 10 |
| | GN = KNG1 PE = 1 SV = 2 | | |
| LU_HUMAN | Lutheran blood group glycoprotein | 1 | 7 |
| | precursor - Homo sapiens | | |
| MARCS_HUMAN | Myristoylated alanine-rich C-kinase | 1 | 3 |
| | substrate OS = Homo sapiens | | |
| | GN = MARCKS PE = 1 SV = 4 | | |
| MLRV_HUMAN | Myosin regulatory light chain 2, | 1 | 43 |
| | ventricular/cardiac muscle isoform | | |
| | OS = Homo sapiens GN = MYL2 PE = 1 | | |
| | SV = 3 | | |
| MRLC2_HUMAN | Myosin regulatory light chain MRLC2 | 0.93 | 2 |
| | OS = Homo sapiens GN = MYLC2B PE = 1 | | |
| | SV = 2 | | |
| MYOZ2_HUMAN | Myozenin-2 OS = Homo sapiens | 1 | 38 |
| | GN = MYOZ2 PE = 1 SV = 1 | | |
| MYPT1_HUMAN | Protein phosphatase 1 regulatory | 0 | 1 |
| | subunit 12A OS = Homo sapiens | | |
| | GN = PPP1R12A PE = 1 SV = 1 | | |
| MYPT2_HUMAN | Protein phosphatase 1 regulatory | 1 | 4 |
| | subunit 12B OS = Homo sapiens | | |
| | GN = PPP1R12B PE = 1 SV = 2 | | |
| NCAM1_HUMAN | Neural cell adhesion molecule 1 | 1 | 3 |
| | OS = Homo sapiens GN = NCAM1 PE = 1 | | |
| | SV = 3 | | |
| NEBL_HUMAN | Nebulette OS = Homo sapiens | 1 | 7 |
| | GN = NEBL PE = 1 SV = 1 | | |
| NEXN_HUMAN | Nexilin OS = Homo sapiens GN = NEXN | 0.98 | 2 |
| | PE = 1 SV = 1 | | |
| NP1L4_HUMAN | Nucleosome assembly protein 1-like | 0.98 | 2 |
| | 4 OS = Homo sapiens GN = NAP1L4 | | |
| | PE = 1 SV = 1 | | |
| OCAD1_HUMAN | OClA domain-containing protein 1 | 1 | 6 |
| | OS = Homo sapiens GN = OClAD1 PE = 1 | | |
| | SV = 1 | | |
| ODPA_HUMAN | Pyruvate dehydrogenase E1 | 1 | 11 |
| | component subunit alpha, somatic | | |
| | form, mitochondrial OS = Homo sapiens | | |
| | GN = PDHA1 PE = 1 SV = 3 | | |
| PEBP1_HUMAN | Phosphatidylethanolamine-binding | 1 | 16 |
| | protein 1 OS = Homo sapiens | | |
| | GN = PEBP1 PE = 1 SV = 3 | | |
| PGRC2_HUMAN | Membrane-associated progesterone | 0 | 1 |
| | receptor component 2 OS = Homo sapiens | | |
| | GN = PGRMC2 PE = 1 SV = 1 | | |
| POPD1_HUMAN | Blood vessel epicardial substance | 0.94 | 1 |
| | OS = Homo sapiens GN = BVES PE = 2 | | |
| | SV = 1 | | |
| PTRF_HUMAN | Polymerase I and transcript release | 1 | 19 |
| | factor OS = Homo sapiens GN = PTRF | | |
| | PE = 1 SV = 1 | | |
| QCR6_HUMAN | Cytochrome b-c1 complex subunit 6, | 1 | 11 |
| | mitochondrial OS = Homo sapiens | | |
| | GN = UQCRH PE = 1 SV = 2 | | |
| RLA2_HUMAN | 60S acidic ribosomal protein P2 | 1 | 7 |
| | OS = Homo sapiens GN = RPLP2 PE = 1 | | |
| | SV = 1 | | |
| ROA3_HUMAN | Heterogeneous nuclear | 0 | 1 |
| | ribonucleoprotein A3 OS = | | |
| | Homo sapiens GN = HNRNPA3 PE = 1 SV = 2 | | |
| SDPR_HUMAN | Serum deprivation-response protein | 1 | 14 |
| | OS = Homo sapiens GN = SDPR PE = 1 | | |
| | SV = 3 | | |
| SRBS2_HUMAN | Sorbin and SH3 domain-containing | 0.96 | 1 |
| | protein 2 OS = Homo sapiens | | |
| | GN = SORBS2 PE = 1 SV = 3 | | |
| SRCH_HUMAN | Sarcoplasmic reticulum histidine-rich | 1 | 23 |
| | calcium-binding protein OS = Homo sapiens | | |
| | GN = HRC PE = 2 SV = 1 | | |
| TEBP_HUMAN | Prostaglandin E synthase 3 OS = | 1 | 4 |
| | Homo sapiens GN = PTGES3 PE = 1 SV = 1 | | |
| TELT_HUMAN | Telethonin OS = Homo sapiens | 1 | 4 |
| | GN = TCAP PE = 1 SV = 1 | | |
| TNNC1_HUMAN | Troponin C, slow skeletal and cardiac | 1 | 42 |
| | muscles OS = Homo sapiens | | |
| | GN = TNNC1 PE = 1 SV = 1 | | |
| TNNI3_HUMAN | Troponin I, cardiac muscle OS = | 1 | 41 |
| | Homo sapiens GN = TNNI3 PE = 1 SV = 3 | | |
| TNNT2_HUMAN | Troponin T, cardiac muscle OS = | 1 | 66 |
| | Homo sapiens GN = TNNT2 PE = 1 SV = 3 | | |
| TPIS_HUMAN | Triosephosphate isomerase | 1 | 23 |
| | OS = Homo sapiens GN = TPI1 PE = 1 | | |
| | SV = 2 | | |
| TPM1_HUMAN | Tropomyosin alpha-1 chain OS = | 1 | 73 |
| | Homo sapiens GN = TPM1 PE = 1 SV = 2 | | |
| TPM2_HUMAN | Tropomyosin beta chain OS = | 1 | 62 |
| | Homo sapiens GN = TPM2 PE = 1 SV = 1 | | |
| TPPP_HUMAN | Tubulin polymerization-promoting | 0.83 | 1 |
| | protein OS = Homo sapiens GN = TPPP | | |
| | PE = 1 SV = 1 | | |
| VDAC1_HUMAN | Voltage-dependent anion-selective | 1 | 10 |
| | channel protein 1 OS = Homo sapiens | | |
| | GN = VDAC1 PE = 1 SV = 2 | | |
| VDAC2_HUMAN | Voltage-dependent anion-selective | 1 | 14 |
| | channel protein 2 OS = Homo sapiens | | |
| | GN = VDAC2 PE = 1 SV = 2 | | |
| VIME_HUMAN | Vimentin OS = Homo sapiens GN = VIM | 1 | 18 |
| | PE = 1 SV = 4 | | |

| | NIF v IF | NIF v IF | IF v NF | IF v NF | NIF v NF | NIF v NF |
|---|---|---|---|---|---|---|
| Primary Protein | Fold | p-value | Fold | p-value | Fold | p-value |
| Name | Change | (ANOVA) | Change | (ANOVA) | Change | (ANOVA) |
| ALBU_HUMAN | 1.07 | 0.6795 | 1.36 | 5.0222E−07 | 1.46 | 1.8704E−06 |
| ALDOA_HUMAN | 1.10 | 0.8171 | −1.25 | 0.4097 | −1.13 | 0.5405 |
| ANT3_HUMAN | −1.32 | 0.4301 | 1.27 | 0.4259 | −1.04 | 0.794 |
| BASI_HUMAN | −1.13 | 0.8077 | −1.19 | 0.274 | −1.35 | 0.0495 |
| CALD1_HUMAN | 1.17 | 0.6795 | 1.03 | 0.8858 | 1.21 | 0.1439 |
| CAPZB_HUMAN | 1.02 | 0.9715 | −1.07 | 0.8399 | −1.05 | 0.8249 |
| CASQ2_HUMAN | −1.23 | 0.3175 | −1.03 | 0.84 | −1.27 | 0.0289 |
| CRIP2_HUMAN | −1.00 | 0.9715 | 1.06 | 0.7313 | 1.05 | 0.5936 |
| CRYAB_HUMAN | −1.10 | 0.7497 | −1.02 | 0.8791 | −1.12 | 0.2523 |
| CSPG2_HUMAN | 1.50 | 0.8671 | 1.58 | 0.4856 | 2.37 | 0.1351 |
| CSRP3_HUMAN | −1.11 | 0.7135 | −1.00 | 0.9957 | −1.12 | 0.5434 |
| CYC_HUMAN | −1.03 | 0.945 | −1.17 | 0.1627 | −1.20 | 0.2564 |
| DESM_HUMAN | 1.15 | 0.8696 | 1.07 | 0.8316 | 1.23 | 0.3995 |
| DSG2_HUMAN | 1.08 | 0.9355 | −1.27 | 0.4892 | −1.18 | 0.53 |
| EF1B_HUMAN | −1.13 | 0.6795 | 1.10 | 0.4508 | −1.03 | 0.8607 |
| FBLN1_HUMAN | 1.13 | 0.8879 | 1.90 | 0.0279 | 2.16 | 0.0468 |
| FETUA_HUMAN | 1.03 | 0.9404 | 1.66 | 1.0691E−11 | 1.71 | 1.3418E−06 |
| FHL2_HUMAN | −1.02 | 0.9871 | −1.19 | 0.4899 | −1.22 | 0.4453 |
| FRIH_HUMAN | 1.51 | 0.1996 | −1.78 | 0.0067 | −1.18 | 0.4597 |
| G3P_HUMAN | −1.02 | 0.9832 | −1.06 | 0.732 | −1.08 | 0.6122 |
| H12_HUMAN | 1.03 | 0.945 | −1.10 | 0.5358 | −1.07 | 0.53 |
| H31_HUMAN | 1.21 | 0.7858 | 1.01 | 0.9014 | 1.22 | 0.3426 |
| HNRPD_HUMAN | 1.07 | 0.6795 | −1.16 | 0.045 | −1.09 | 0.0464 |
| HP1B3_HUMAN | −1.03 | 0.945 | −1.18 | 0.2797 | −1.21 | 0.1298 |
| HS90A_HUMAN | 1.05 | 0.945 | −1.07 | 0.8024 | −1.02 | 0.8654 |
| HS90B_HUMAN | 1.06 | 0.9319 | −1.15 | 0.6588 | −1.08 | 0.7662 |
| HSPB1_HUMAN | −1.10 | 0.6795 | 1.06 | 0.5118 | −1.04 | 0.7432 |
| HSPB7_HUMAN | −1.15 | 0.3444 | −1.15 | 0.4175 | −1.33 | 0.0108 |
| ICAL_HUMAN | −1.40 | 0.3444 | 1.06 | 0.8083 | −1.31 | 0.0761 |
| KAD1_HUMAN | 1.12 | 0.2107 | −1.26 | 0.036 | −1.12 | 0.2405 |
| KCRM_HUMAN | −1.23 | 0.6795 | −1.41 | 0.2813 | −1.73 | 0.0045 |
| KCRS_HUMAN | 1.03 | 0.9715 | −1.28 | 0.4773 | −1.24 | 0.2599 |
| KNG1_HUMAN | −1.16 | 0.726 | 1.67 | 0.0042 | 1.44 | 0.1063 |
| LU_HUMAN | −1.07 | 0.8696 | −1.17 | 0.0735 | −1.26 | 0.0464 |
| MARCS_HUMAN | −1.16 | 0.8671 | 1.06 | 0.8316 | −1.10 | 0.6902 |
| MLRV_HUMAN | 1.11 | 0.7398 | −1.29 | 0.0028 | −1.15 | 0.2511 |
| MRLC2_HUMAN | 1.01 | 0.9871 | 1.24 | 0.3137 | 1.26 | 0.1111 |
| MYOZ2_HUMAN | 1.02 | 0.9585 | −1.16 | 0.4899 | −1.13 | 0.4383 |
| MYPT1_HUMAN | −1.28 | 0.5264 | 1.03 | 0.8269 | −1.24 | 0.2222 |
| MYPT2_HUMAN | −1.13 | 0.8797 | −1.23 | 0.2564 | −1.39 | 0.1755 |
| NCAM1_HUMAN | −1.22 | 0.6833 | −1.20 | 0.7056 | −1.46 | 0.0587 |
| NEBL_HUMAN | 1.30 | 0.7135 | −1.23 | 0.5516 | 1.06 | 0.9396 |
| NEXN_HUMAN | 1.19 | 0.7891 | −1.24 | 0.4266 | −1.04 | 0.9396 |
| NP1L4_HUMAN | −1.06 | 0.8671 | −1.02 | 0.9585 | −1.08 | 0.3465 |
| OCAD1_HUMAN | −1.02 | 0.945 | −1.04 | 0.84 | −1.05 | 0.6902 |
| ODPA_HUMAN | 1.05 | 0.9355 | −1.23 | 0.4579 | −1.16 | 0.5046 |
| PEBP1_HUMAN | −1.08 | 0.5535 | −1.16 | 0.0084 | −1.25 | 0.00035967 |
| PGRC2_HUMAN | −1.07 | 0.7451 | −1.15 | 0.2085 | −1.23 | 0.0299 |
| POPD1_HUMAN | −1.20 | 0.8431 | −1.17 | 0.5684 | −1.40 | 0.1458 |
| PTRF_HUMAN | 1.07 | 0.7269 | −1.08 | 0.4899 | −1.02 | 0.9243 |
| QCR6_HUMAN | −1.17 | 0.7858 | −1.36 | 0.0028 | −1.59 | 0.0409 |
| RLA2_HUMAN | −1.16 | 0.6795 | −1.18 | 0.2688 | −1.36 | 0.001 |
| ROA3_HUMAN | 1.09 | 0.7845 | −1.18 | 0.3061 | −1.08 | 0.5263 |
| SDPR_HUMAN | −1.08 | 0.6795 | −1.03 | 0.4899 | −1.12 | 0.1169 |
| SRBS2_HUMAN | 1.44 | 0.6511 | 1.01 | 0.94 | 1.46 | 0.2048 |
| SRCH_HUMAN | −1.36 | 0.3288 | 1.02 | 0.8918 | −1.33 | 0.0648 |
| TEBP_HUMAN | −1.16 | 0.6232 | −1.10 | 0.6071 | −1.28 | 0.0217 |
| TELT_HUMAN | 1.11 | 0.7891 | −1.51 | 0.00072989 | −1.36 | 0.0225 |
| TNNC1_HUMAN | −1.12 | 0.7105 | −1.22 | 0.0023 | −1.36 | 0.0024 |
| TNNI3_HUMAN | −1.05 | 0.9129 | −1.13 | 0.0616 | −1.19 | 0.1662 |
| TNNT2_HUMAN | −1.16 | 0.5784 | −1.23 | 0.0223 | −1.42 | 0.000011964 |
| TPIS_HUMAN | 1.11 | 0.6795 | −1.22 | 0.2025 | −1.10 | 0.4383 |
| TPM1_HUMAN | −1.09 | 0.5797 | −1.16 | 0.0163 | −1.27 | 0.00024801 |
| TPM2_HUMAN | −1.10 | 0.3398 | −1.23 | 0.0027 | −1.35 | 7.7343E−06 |
| TPPP_HUMAN | −1.04 | 0.9639 | −1.41 | 0.1662 | −1.47 | 0.1301 |
| VDAC1_HUMAN | 1.01 | 0.9715 | −1.17 | 0.4892 | −1.16 | 0.2171 |
| VDAC2_HUMAN | −1.03 | 0.945 | −1.17 | 0.3298 | −1.21 | 0.0838 |
| VIME_HUMAN | 1.33 | 0.726 | 1.16 | 0.6925 | 1.54 | 0.1411 |

TABLE 10
DIFFERENTIAL PHOSPHORYLATION OF PROTEINS IN NIF AND IF HEART

| | | | NIF v IF |
|---|---|---|---|
| Primary Protein | | | Fold |
| Name | Protein Description | Modified Peptide Sequence | Change |
| ALBU_HUMAN | Serum albumin precursor - | TC[160.0307]VADES[166.9984] | 1.27 |
| | Homo sapiens (Human) | AENC[160.0307]DK | |
| ALDOA_HUMAN | Fructose-bisphosphate aldolase A - | GILAADES[166.9984]TGSIAK | −1.86 |
| | Homo sapiens (Human) | | |
| ALDOA_HUMAN | Fructose-bisphosphate aldolase A - | GILAADESTGS[166.9984]IAK | −1.38 |
| | Homo sapiens (Human) | | |
| ALDOA_HUMAN | Fructose-bisphosphate aldolase A - | GILAADESTGS[166.9984]IAKR | 1.43 |
| | Homo sapiens (Human) | | |
| ANT3_HUMAN | Antithrombin-III OS = Homo sapiens | ATEDEGS[166.9984]EQKIPEATN | −1.40 |
| | GN = SERPINC1 PE = 1 SV = 1 | R | |
| ANT3_HUMAN | Antithrombin-III OS = Homo sapiens | KATEDEGS[166.9984]EQKIPEAT | −1.50 |
| | GN = SERPINC1 PE = 1 SV = 1 | NR | |
| BASI_HUMAN | Basigin precursor - Homo sapiens | KPEDVLDDDDAGSAPLKSS[166.9 | −7.27 |
| | (Human) | 984]GQHQNDK | |
| BASI_HUMAN | Basigin precursor - Homo sapiens | RKPEDVLDDDDAGS[166.9984]A | 1.27 |
| | (Human) | PLK | |
| BASI_HUMAN | Basigin precursor - Homo sapiens | RKPEDVLDDDDAGSAPLKS[166.9 | −5.36 |
| | (Human) | 984]SGQHQNDK | |
| BASI_HUMAN | Basigin precursor - Homo sapiens | RKPEDVLDDDDAGSAPLKSS[166. | −4.36 |
| | (Human) | 9984]GQHQNDK | |
| CALD1_HUMAN | Caldesmon OS = Homo sapiens | RGS[166.9984]IGENQVEVM[14 | 1.79 |
| | GN = CALD1 PE = 1 SV = 2 | 7.0354]VEEK | |
| CALD1_HUMAN | Caldesmon OS = Homo sapiens | TPDGNKS[166.9984]PAPKPSDL | 1.62 |
| | GN = CALD1 PE = 1 SV = 2 | RPGDVSSK | |
| CAPZB_HUMAN | F-actin-capping protein subunit | ELS[166.9984]QVLTQR | −2.24 |
| | beta - Homo sapiens (Human) | | |
| CASQ2_HUMAN | Calsequestrin-2 OS = Homo sapiens | KYDLLC[160.0307]LYYHEPVS[16 | −2.48 |
| | GN = CASQ2 PE = 1 SV = 2 | 6.9984]SDKVTQK | |
| CRIP2_HUMAN | Cysteine-rich protein 2 - | ASS[166.9984]VTTFTGEPNTC[1 | −1.61 |
| | Homo sapiens (Human) | 60.0307]PR | |
| CRYAB_HUMAN | Alpha-crystallin B chain OS = | RPFFPFHSPS[166.9984]R | −1.64 |
| | Homo sapiens GN = CRYAB PE = 1 | | |
| | SV = 2 | | |
| CSPG2_HUMAN | Versican core protein OS = | TDGQVS[166.9984]GEAIK | 2.31 |
| | Homo sapiens GN = VCAN PE = 1 | | |
| | SV = 3 | | |
| CSRP3_HUMAN | Cysteine and glycine-rich protein 3 | FGES[166.9984]EKC[160.0307]P | −1.65 |
| | OS = Homo sapiens GN = CSRP3 PE = 1 | R | |
| | SV = 1 | | |
| CSRP3_HUMAN | Cysteine and glycine-rich protein 3 | GIGYGQGAGC[160.0307]LST[18 | −1.25 |
| | OS = Homo sapiens GN = CSRP3 PE = 1 | 1.014]DTGEHLGLQFQQSPKPAR | |
| | SV = 1 | | |
| CSRP3_HUMAN | Cysteine and glycine-rich protein 3 | GIGYGQGAGC[160.0307]LSTDT | −1.13 |
| | OS = Homo sapiens GN = CSRP3 PE = 1 | GEHLGLQFQQS[166.9984]PKPA | |
| | SV = 1 | R | |
| CSRP3_HUMAN | Cysteine and glycine-rich protein 3 | S[166.9984]LESTNVTDKDGELYC | −1.12 |
| | OS = Homo sapiens GN = CSRP3 PE = 1 | [160.0307]K | |
| | SV = 1 | | |
| CYC_HUMAN | Cytochrome c - Homo sapiens | KTGQAPGYS[166.9984]YTAANK | 1.05 |
| | (Human) | | |
| CYC_HUMAN | Cytochrome c - Homo sapiens | KTGQAPGYSYT[181.014]AANK | 1.78 |
| | (Human) | | |
| CYC_HUMAN | Cytochrome c - Homo sapiens | TGQAPGYS[166.9984]YTAANK | 1.33 |
| | (Human) | | |
| DESM_HUMAN | Desmin - Homo sapiens (Human) | TFGGAPGFPLGS[166.9984]PLSS | 1.50 |
| | | [166.9984]PVFPR | |
| DESM_HUMAN | Desmin - Homo sapiens (Human) | TFGGAPGFPLGS[166.9984]PLSS | 2.16 |
| | | PVFPR | |
| DESM_HUMAN | Desmin - Homo sapiens (Human) | TFGGAPGFPLGSPLSS[166.9984] | 1.41 |
| | | PVFPR | |
| DSG2_HUMAN | Desmoglein-2 OS = Homo sapiens | VVPSFLPVDQGGS[166.9984]LV | 1.77 |
| | GN = DSG2 PE = 1 SV = 2 | GR | |
| DSG2_HUMAN | Desmoglein-2 OS = Homo sapiens | WEEHRS[166.9984]LLSGR | 2.33 |
| | GN = DSG2 PE = 1 SV = 2 | | |
| EF1B_HUMAN | Elongation factor 1-beta - | YGPADVEDTTGSGATDSKDDDDI | 1.08 |
| | Homo sapiens (Human) | DLFGS[166.9984]DDEEESEEAKR | |
| FBN1_HUMAN | Fibrillin-1 OS = Homo sapiens | GNPEPPVS[166.9984]GEM[147. | 3.80 |
| | GN = FBN1 PE = 1 SV = 1 | 0354]DDNSLSPEAC[160.0307]Y | |
| | | EC[160.0307]K | |
| FETUA_HUMAN | Alpha-2-HS-glycoprotein precursor - | C[160.0307]DSSPDS[166.9984] | −2.17 |
| | Homo sapiens (Human) | AEDVR | |
| FETUA_HUMAN | Alpha-2-HS-glycoprotein precursor - | C[160.0307]DSSPDS[166.9984] | −2.09 |
| | Homo sapiens (Human) | AEDVRK | |
| FETUA_HUMAN | Alpha-2-HS-glycoprotein precursor - | HTFM[147.0354]GVVSLGSPS[16 | 2.41 |
| | Homo sapiens (Human) | 6.9984]GEVSHPR | |
| FETUA_HUMAN | Alpha-2-HS-glycoprotein precursor - | HTFMGVVSLGSPS[166.9984]GE | −2.32 |
| | Homo sapiens (Human) | VSHPR | |
| FETUA_HUMAN | Alpha-2-HS-glycoprotein precursor - | HTFMGVVSLGSPSGEVS[166.998 | −1.88 |
| | Homo sapiens (Human) | 4]HPR | |
| FHL2_HUMAN | Four and a half LIM domains | YIS[166.9984]FEER | −3.34 |
| | protein 2 OS = Homo sapiens | | |
| | GN = FHL2 PE = 1 SV = 3 | | |
| FRIH_HUMAN | Ferritin heavy chain OS = | KMGAPESGLAEYLFDKHTLGDS[1 | −1.39 |
| | Homo sapiens GN = FTH1 PE = 1 | 66.9984]DNES | |
| | SV = 2 | | |
| G3P_HUMAN | Glyceraldehyde-3-phosphate | GALQNIIPAS[166.9984]TGAAK | −1.32 |
| | dehydrogenase - Homo sapiens | | |
| | (Human) | | |
| G3P_HUMAN | Glyceraldehyde-3-phosphate | IISNASC[160.0307]T[181.014]T | −1.27 |
| | dehydrogenase - Homo sapiens | NC[160.0307]LAPLAK | |
| | (Human) | | |
| G3P_HUMAN | Glyceraldehyde-3-phosphate | VIHDNFGIVEGLM[147.0354]TT | −1.16 |
| | dehydrogenase - Homo sapiens | VHAITAT[181.014]QK | |
| | (Human) | | |
| G3P_HUMAN | Glyceraldehyde-3-phosphate | VIHDNFGIVEGLMTTVHAITAT[18 | −1.56 |
| | dehydrogenase - Homo sapiens | 1.014]QK | |
| | (Human) | | |
| H12_HUMAN | Histone H1.2 - Homo sapiens | S[166.9984]ETAPAAPAAAPPAEK | 1.31 |
| | (Human) | | |
| H31_HUMAN | Histone H3.1 OS = Homo sapiens | ST[181.014]ELLIR | 1.39 |
| | GN = HIST1H3A PE = 1 SV = 2 | | |
| HNRPD_HUMAN | Heterogeneous nuclear | IDASKNEEDEGHSNS[166.9984]S | 1.86 |
| | ribonucleoprotein D0 - | PR | |
| | Homo sapiens (Human) | | |
| HNRPD_HUMAN | Heterogeneous nuclear | IDASKNEEDEGHSNSS[166.9984] | 1.60 |
| | ribonucleoprotein D0 - | PR | |
| | Homo sapiens (Human) | | |
| HP1B3_HUMAN | Heterochromatin protein 1-binding | TVNSTRET[181.014]PPK | 1.45 |
| | protein 3 - Homo sapiens (Human) | | |
| HS90A_HUMAN | Heat shock protein HSP 90-alpha - | ES[166.9984]EDKPEIEDVGSDEE | −3.42 |
| | Homo sapiens (Human) | EEKK | |
| HS90A_HUMAN | Heat shock protein HSP 90-alpha - | ES[166.9984]EDKPEIEDVGSDEE | −1.09 |
| | Homo sapiens (Human) | EEKKDGDK | |
| HS90A_HUMAN | Heat shock protein HSP 90-alpha - | ESEDKPEIEDVGS[166.9984]DEE | 1.07 |
| | Homo sapiens (Human) | EEKKDGDK | |
| HS90B_HUMAN | Heat shock protein HSP 90-beta - | IEDVGS[166.9984]DEEDDSGK | 2.71 |
| | Homo sapiens (Human) | | |
| HS90B_HUMAN | Heat shock protein HSP 90-beta - | IEDVGS[166.9984]DEEDDSGKD | 1.29 |
| | Homo sapiens (Human) | K | |
| HS90B_HUMAN | Heat shock protein HSP 90-beta - | IEDVGS[166.9984]DEEDDSGKD | 1.55 |
| | Homo sapiens (Human) | KK | |
| HSPB1_HUMAN | Heat shock protein beta-1 - | GPS[166.9984]WDPFR | −4.01 |
| | Homo sapiens (Human) | | |
| HSPB1_HUMAN | Heat shock protein beta-1 - | GPS[166.9984]WDPFRDWYPHS | −3.32 |
| | Homo sapiens (Human) | R | |
| HSPB1_HUMAN | Heat shock protein beta-1 - | QLS[166.9984]SGVSEIR | −3.99 |
| | Homo sapiens (Human) | | |
| HSPB7_HUMAN | Heat shock protein beta-7 | AERS[166.9984]FHSSSSSSSSSTS | −2.80 |
| | OS = Homo sapiens GN = HSPB7 PE = | SSASR | |
| | 1 SV = 1 | | |
| HSPB7_HUMAN | Heat shock protein beta-7 | AERSFHSSSSSSSSS[166.9984]TS | −3.08 |
| | OS = Homo sapiens GN = HSPB7 PE = 1 | SSASR | |
| | SV = 1 | | |
| HSPB7_HUMAN | Heat shock protein beta-7 | S[166.9984]FHSSSSSSSSSTSSSA | −3.46 |
| | OS = Homo sapiens GN = HSPB7 PE = 1 | SR | |
| | SV = 1 | | |
| HSPB7_HUMAN | Heat shock protein beta-7 | SFHS[166.9984]S[166.9984]SSS | −3.20 |
| | OS = Homo sapiens GN = HSPB7 PE = 1 | SSSSTSSSASR | |
| | SV = 1 | | |
| HSPB7_HUMAN | Heat shock protein beta-7 | SFHS[166.9984]SSSSSSSSTSSSA | −2.56 |
| | OS = Homo sapiens GN = HSPB7 PE = 1 | SR | |
| | SV = 1 | | |
| ICAL_HUMAN | Calpastatin OS = Homo sapiens | EGITGPPADSSKPIGPDDAIDALSS | −1.07 |
| | GN = CAST PE = 1 SV = 4 | DFTC[160.0307]GS[166.9984]P | |
| | | TAAGK | |
| ICAL_HUMAN | Calpastatin OS = Homo sapiens | EGITGPPADSSKPIGPDDAIDALSS | −1.16 |
| | GN = CAST PE = 1 SV = 4 | DFTC[160.0307]GSPT[181.014] | |
| | | AAGK | |
| ICAL_HUMAN | Calpastatin OS = Homo sapiens | KEGITGPPADSSKPIGPDDAIDALS | 1.20 |
| | GN = CAST PE = 1 SV = 4 | SDFTC[160.0307]GS[166.9984] | |
| | | PTAAGK | |
| KAD1_HUMAN | Adenylate kinase isoenzyme 1 | KVNAEGS[166.9984]VDSVFSQV | 1.46 |
| | OS = Homo sapiens GN = AK1 PE = 1 | C[160.0307]THLDALK | |
| | SV = 3 | | |
| KAD1_HUMAN | Adenylate kinase isoenzyme 1 | VNAEGS[166.9984]VDSVFSQVC | −1.06 |
| | OS = Homo sapiens GN = AK1 PE = 1 | [160.0307]THLDALK | |
| | SV = 3 | | |
| KAD1_HUMAN | Adenylate kinase isoenzyme 1 | YGYTHLS[166.9984]TGDLLR | 1.58 |
| | OS = Homo sapiens GN = AK1 PE = 1 | | |
| | SV = 3 | | |
| KCRM_HUMAN | Creatine kinase M-type OS = | GQS[166.9984]IDDMIPAQK | −1.19 |
| | Homo sapiens GN = CKM PE = 1 SV = 2 | | |
| KCRM_HUMAN | Creatine kinase M-type OS = | GTGGVDTAAVGS[166.9984]VFD | −2.11 |
| | Homo sapiens GN = CKM PE = 1 SV = 2 | VSNADR | |
| KCRM_HUMAN | Creatine kinase M-type OS = | RGT[181.014]GGVDTAAVGSVFD | −1.24 |
| | Homo sapiens GN = CKM PE = 1 SV = 2 | VSNADR | |
| KCRM_HUMAN | Creatine kinase M-type OS = | RGTGGVDTAAVGS[166.9984]VF | −1.42 |
| | Homo sapiens GN = CKM PE = 1 SV = 2 | DVSNADR | |
| KCRS_HUMAN | Creatine kinase, sarcomeric | LGYILTC[160.0307]PS[166.9984] | 1.30 |
| | mitochondrial OS = Homo sapiens | NLGTGLR | |
| | GN = CKMT2 PE = 1 SV = 2 | | |
| KNG1_HUMAN | Kininogen-1 OS = Homo sapiens | ETTC[160.0307]S[166.9984]KES | −1.30 |
| | GN = KNG1 PE = 1 SV = 2 | NEELTESC[160.0307]ETK | |
| KNG1_HUMAN | Kininogen-1 OS = Homo sapiens | ETTC[160.0307]S[166.9984]KES | −1.05 |
| | GN = KNG1 PE = 1 SV = 2 | NEELTESC[160.0307]ETKK | |
| KNG1_HUMAN | Kininogen-1 OS = Homo sapiens | ETTC[160.0307]SKES[166.9984] | −1.39 |
| | GN = KNG1 PE = 1 SV = 2 | NEELTESC[160.0307]ETK | |
| KNG1_HUMAN | Kininogen-1 OS = Homo sapiens | ETTC[160.0307]SKES[166.9984] | −1.10 |
| | GN = KNG1 PE = 1 SV = 2 | NEELTESC[160.0307]ETKK | |
| LU_HUMAN | Lutheran blood group glycoprotein | GAPPPGEPGLS[166.9984]HSGS | 1.18 |
| | precursor - Homo sapiens (Human) | EQPEQJGLLM[147.0354]GGAS | |
| | | GGAR | |
| LU_HUMAN | Lutheran blood group glycoprotein | GAPPPGEPGLSHS[166.9984]GS | −2.76 |
| | precursor - Homo sapiens (Human) | EQPEQTGLLMGGASGGAR | |
| MARCS_HUMAN | Myristoylated alanine-rich C-kinase | AEDGATPSPSNET[181.014]PK | 1.84 |
| | substrate - Homo sapiens (Human) | | |
| MARCS_HUMAN | Myristoylated alanine-rich C-kinase | AEDGATPSPSNET[181.014]PKK | 1.61 |
| | substrate - Homo sapiens (Human) | | |
| MARCS_HUMAN | Myristoylated alanine-rich C-kinase | EAPAEGEAAEPGS[166.9984]PTA | −1.99 |
| | substrate - Homo sapiens (Human) | AEGEAASAASSTSSPK | |
| MARCS_HUMAN | Myristoylated alanine-rich C-kinase | GEPAAAAAPEAGAS[166.9984]P | −2.16 |
| | substrate - Homo sapiens (Human) | VEK | |
| MARCS_HUMAN | Myristoylated alanine-rich C-kinase | LSGFS[166.9984]FK | 2.15 |
| | substrate - Homo sapiens (Human) | | |
| MARCS_HUMAN | Myristoylated alanine-rich C-kinase | LSGFS[166.9984]FKK | 1.89 |
| | substrate - Homo sapiens (Human) | | |
| MLRV_HUMAN | Myosin regulatory light chain 2, | AGGANS[166.9984]NVFSM[147 | 2.86 |
| | ventricular/cardiac muscle isoform | .0354]FEQTQIQEFK | |
| | OS = Homo sapiens GN = MYL2 PE = 1 | | |
| | SV = 3 | | |
| MLRV_HUMAN | Myosin regulatory light chain 2, | AGGANS[166.9984]NVFSMFEQ | −3.53 |
| | ventricular/cardiac muscle isoform | TQIQEFK | |
| | OS = Homo sapiens GN = MYL2 PE = 1 | | |
| | SV = 3 | | |
| MLRV_HUMAN | Myosin regulatory light chain 2, | RAGGANS[166.9984]NVFSM[14 | 3.01 |
| | ventricular/cardiac muscle isoform | 7.0354]FEQTQIQEFK | |
| | OS = Homo sapiens GN = MYL2 PE = 1 | | |
| | SV = 3 | | |
| MLRV_HUMAN | Myosin regulatory light chain 2, | RAGGANS[166.9984]NVFSMFE | −2.29 |
| | ventricular/cardiac muscle isoform | QTQIQEFK | |
| | OS = Homo sapiens GN = MYL2 PE = 1 | | |
| | SV = 3 | | |
| MYOZ2_HUMAN | Myozenin-2 OS = Homo sapiens | DIMLEELSHLS[166.9984]NR | −1.26 |
| | GN = MYOZ2 PE = 1 SV = 1 | | |
| MYOZ2_HUMAN | Myozenin-2 OS = Homo sapiens | S[166.9984]PPNPDNIAPGYSGPL | 1.20 |
| | GN = MYOZ2 PE = 1 SV = 1 | K | |
| MYOZ2_HUMAN | Myozenin-2 OS = Homo sapiens | VDGSNLEGGS[166.9984]QQAPL | 1.60 |
| | GN = MYOZ2 PE = 1 SV = 1 | TPPNTPDPR | |
| MYOZ2_HUMAN | Myozenin-2 OS = Homo sapiens | VDGSNLEGGSQQAPLT[181.014] | −1.53 |
| | GN = MYOZ2 PE = 1 SV = 1 | PPNT[181.014]PDPR | |
| MYOZ2_HUMAN | Myozenin-2 OS = Homo sapiens | VDGSNLEGGSQQAPLT[181.014] | −1.03 |
| | GN = MYOZ2 PE = 1 SV = 1 | PPNTPDPR | |
| MYPT1_HUMAN | Protein phosphatase 1 regulatory | KTGS[166.9984]YGALAEITASK | 1.21 |
| | subunit 12A OS = Homo sapiens | | |
| | GN = PPP1R12A PE = 1 SV = 1 | | |
| MYPT1_HUMAN | Protein phosphatase 1 regulatory | KTGSY[243.0297]GALAEITASK | 1.41 |
| | subunit 12A OS = Homo sapiens | | |
| | GN = PPP1R12A PE = 1 SV = 1 | | |
| MYPT1_HUMAN | Protein phosphatase 1 regulatory | RS[166.9984]TQGVTLTDLQEAEK | −2.68 |
| | subunit 12A OS = Homo sapiens | | |
| | GN = PPP1R12A PE = 1 SV = 1 | | |
| MYPT1_HUMAN | Protein phosphatase 1 regulatory | RST[181.014]QGVTLTDLQEAEK | −1.92 |
| | subunit 12A OS = Homo sapiens | | |
| | GN = PPP1R12A PE = 1 SV = 1 | | |
| MYPT1_HUMAN | Protein phosphatase 1 regulatory | S[166.9984]YLTPVRDEESESQR | −1.02 |
| | subunit 12A OS = Homo sapiens | | |
| | GN = PPP1R12A PE = 1 SV = 1 | | |
| MYPT2_HUMAN | Protein phosphatase 1 regulatory | DEDET[181.014]DGSEEVKETWH | 2.24 |
| | subunit 12B OS = Homo sapiens | ER | |
| | GN = PPP1R12B PE = 1 SV = 2 | | |
| MYPT2_HUMAN | Protein phosphatase 1 regulatory | S[166.9984]LDEEPIC[160.0307] | −2.20 |
| | subunit 12B OS = Homo sapiens | HR | |
| | GN = PPP1R12B PE = 1 SV = 2 | | |
| NCAM1_HUMAN | Neural cell adhesion molecule 1 | AAFSKDES[166.9984]KEPIVEVR | −1.09 |
| | OS = Homo sapiens GN = NCAM1 | | |
| | PE = 1 SV = 3 | | |
| NEBL_HUMAN | Nebulette OS = Homo sapiens | TDPGS[166.9984]IFDLDPLEDNI | 1.58 |
| | GN = NEBL PE = 1 SV = 1 | QSR | |
| NEXN_HUMAN | Nexilin OS = Homo sapiens GN = NEXN | EM[147.0354]LAS[166.9984]DD | 5.55 |
| | PE = 1 SV = 1 | EEDVSSK | |
| NEXN_HUMAN | Nexilin OS = Homo sapiens GN = NEXN | EM[147.0354]LAS[166.9984]DD | 13.46 |
| | PE = 1 SV = 1 | EEDVSSKVEK | |
| NEXN_HUMAN | Nexilin OS = Homo sapiens GN = NEXN | TIS[166.9984]QEFLTPGK | 2.79 |
| | PE = 1 SV = 1 | | |
| NP1L4_HUMAN | Nucleosome assembly protein 1-like | EFITGDVEPTDAESEWHS[166.99 | −1.12 |
| | 4 OS = Homo sapiens GN = NAP1L4 PE = | 84]ENEEEEK | |
| | 1 SV = 1 | | |
| NP1L4_HUMAN | Nucleosome assembly protein 1-like | EFITGDVEPTDAESEWHS[166.99 | −1.46 |
| | 4 OS = Homo sapiens GN = NAP1L4 | 84]ENEEEEKLAGDMK | |
| | PE = 1 SV = 1 | | |
| OCAD1_HUMAN | OClA domain-containing protein 1 | RSS[166.9984]PPGHYYQK | 3.33 |
| | OS = Homo sapiens GN = OClAD1 | | |
| | PE = 1 SV = 1 | | |
| ODPA_HUMAN | ODPA_HUMAN | YGM[147.0354]GT[181.014]SV | 123.00 |
| | | ER | |
| ODPA_HUMAN | Pyruvate dehydrogenase E1 | YHGHS[166.9984]M[147.0354] | 32.46 |
| | component subunit alpha, somatic | SDPGVS[166.9984]YR | |
| | form, mitochondrial OS = | | |
| | Homo sapiens GN = PDHA1 PE = 1 | | |
| | SV = 3 | | |
| ODPA_HUMAN | Pyruvate dehydrogenase E1 | YHGHS[166.9984]M[147.0354] | 25.87 |
| | component subunit alpha, somatic | SDPGVSY[243.0297]R | |
| | form, mitochondrial OS = | | |
| | Homo sapiens GN = PDHA1 PE = 1 | | |
| | SV = 3 | | |
| ODPA_HUMAN | Pyruvate dehydrogenase E1 | YHGHS[166.9984]M[147.0354] | 1.16 |
| | component subunit alpha, somatic | SDPGVSYR | |
| | form, mitochondrial OS = | | |
| | Homo sapiens GN = PDHA1 PE = 1 | | |
| | SV = 3 | | |
| ODPA_HUMAN | Pyruvate dehydrogenase E1 | YHGHS[166.9984]MSDPGVSYR | −2.20 |
| | component subunit alpha, somatic | | |
| | form, mitochondrial OS = | | |
| | Homo sapiens GN = PDHA1 PE = 1 | | |
| | SV = 3 | | |
| ODPA_HUMAN | Pyruvate dehydrogenase E1 | YHGHSMS[166.9984]DPGVSYR | −1.64 |
| | component subunit alpha, somatic | | |
| | form, mitochondrial OS = | | |
| | Homo sapiens GN = PDHA1 PE = 1 | | |
| | SV = 3 | | |
| PEBP1_HUMAN | Phosphatidylethanolamine-binding | NRPTS[166.9984]ISWDGLDSGK | −1.00 |
| | protein 1 - Homo sapiens (Human) | | |
| PEBP1_HUMAN | Phosphatidylethanolamine-binding | NRPTSIS[166.9984]WDGLDSGK | −1.01 |
| | protein 1 - Homo sapiens (Human) | | |
| PGRC2_HUMAN | Membrane-associated | LLKPGEEPS[166.9984]EYTDEED | 1.32 |
| | progesterone receptor component | TKDHNKQD | |
| | 2 - Homo sapiens (Human) | | |
| PGRC2_HUMAN | Membrane-associated | LLKPGEEPSEYT[181.014]DEEDT | −1.17 |
| | progesterone receptor component | K | |
| | 2 - Homo sapiens (Human) | | |
| PGRC2_HUMAN | Membrane-associated | LLKPGEEPSEYT[181.014]DEEDT | −1.53 |
| | progesterone receptor component | KDHNK | |
| | 2 - Homo sapiens (Human) | | |
| PGRC2_HUMAN | Membrane-associated | LLKPGEEPSEYT[181.014]DEEDT | 1.15 |
| | progesterone receptor component | KDHNKQD | |
| | 2 - Homo sapiens (Human) | | |
| POPD1_HUMAN | Blood vessel epicardial substance | GTSS[166.9984]MS[166.9984]S | −8.02 |
| | OS = Homo sapiens GN = BVES PE = 2 | LHVSSPHQR | |
| | SV = 1 | | |
| POPD1_HUMAN | Blood vessel epicardial substance | GTSSM[147.0354]S[166.9984]S | 1.68 |
| | OS = Homo sapiens GN = BVES PE = 2 | LHVSSPHQR | |
| | SV = 1 | | |
| POPD1_HUMAN | Blood vessel epicardial substance | GTSSMSS[166.9984]LHVSSPHQ | −5.94 |
| | OS = Homo sapiens GN = BVES PE = 2 | R | |
| | SV = 1 | | |
| POPD1_HUMAN | Blood vessel epicardial substance | M[147.0354]KPIEEGAEDDDDVF | 2.59 |
| | OS = Homo sapiens GN = BVES PE = 2 | EPAS[166.9984]PNTLK | |
| | SV = 1 | | |
| POPD1_HUMAN | Blood vessel epicardial substance | MKPIEEGAEDDDDVFEPAS[166.9 | −1.69 |
| | OS = Homo sapiens GN = BVES PE = 2 | 984]PNTLK | |
| | SV = 1 | | |
| POPD1_HUMAN | Blood vessel epicardial substance | NS[166.9984]IASSSDSDDGLHQF | −10.06 |
| | OS = Homo sapiens GN = BVES PE = 2 | LR | |
| | SV = 1 | | |
| POPD1_HUMAN | Blood vessel epicardial substance | NSIASSS[166.9984]DSDDGLHQF | 1.59 |
| | OS = Homo sapiens GN = BVES PE = 2 | LR | |
| | SV = 1 | | |
| POPD1_HUMAN | Blood vessel epicardial substance | NSIASSSDS[166.9984]DDGLHQF | 1.41 |
| | OS = Homo sapiens GN = BVES PE = 2 | LR | |
| | SV = 1 | | |
| PTRF_HUMAN | Polymerase I and transcript release | ES[166.9984]EALPEKEGEELGEG | −1.12 |
| | factor OS = Homo sapiens GN = PTRF | ERPEEDAAALELS[166.9984]SDE | |
| | PE = 1 SV = 1 | AVEVEEVIEESR | |
| PTRF_HUMAN | Polymerase I and transcript release | ES[166.9984]EALPEKEGEELGEG | −1.04 |
| | factor OS = Homo sapiens GN = PTRF | ERPEEDAAALELSS[166.9984]DE | |
| | PE = 1 SV = 1 | AVEVEEVIEESR | |
| PTRF_HUMAN | Polymerase I and transcript release | KVS[166.9984]VNVK | 2.15 |
| | factor OS = Homo sapiens GN = PTRF | | |
| | PE = 1 SV = 1 | | |
| PTRF_HUMAN | Polymerase I and transcript release | LPAKLS[166.9984]ISK | 1.27 |
| | factor OS = Homo sapiens GN = PTRF | | |
| | PE = 1 SV = 1 | | |
| PTRF_HUMAN | Polymerase I and transcript release | RGS[166.9984]S[166.9984]PDV | 1.53 |
| | factor OS = Homo sapiens GN = PTRF | HALLEITEESDAVLVDK | |
| | PE = 1 SV = 1 | | |
| PTRF_HUMAN | Polymerase I and transcript release | S[166.9984]FTPDHVVYAR | 1.08 |
| | factor OS = Homo sapiens GN = PTRF | | |
| | PE = 1 SV = 1 | | |
| PTRF_HUMAN | Polymerase I and transcript release | S[166.9984]LKESEALPEK | −1.25 |
| | factor OS = Homo sapiens GN = PTRF | | |
| | PE = 1 SV = 1 | | |
| PTRF_HUMAN | Polymerase I and transcript release | VM[147.0354]IYQDEVKLPAKLSI | 2.28 |
| | factor OS = Homo sapiens GN = PTRF | S[166.9984]K | |
| | PE = 1 SV = 1 | | |
| QCR6_HUMAN | Cytochrome b-c1 complex subunit | S[166.9984]HTEEDC[160.0307] | −1.38 |
| | 6, mitochondrial precursor - | TEELFDFLHAR | |
| | Homo sapiens (Human) | | |
| QCR6_HUMAN | Cytochrome b-c1 complex subunit | SHT[181.014]EEDC[160.0307]T | −1.08 |
| | 6, mitochondrial precursor - | EELFDFLHAR | |
| | Homo sapiens (Human) | | |
| RLA2_HUMAN | 60S acidic ribosomal protein P2 | KEES[166.9984]EES[166.9984]D | 3.57 |
| | OS = Homo sapiens GN = RPLP2 PE = 1 | DDM[147.0354]GFGLFD | |
| | SV = 1 | | |
| RLA2_HUMAN | 60S acidic ribosomal protein P2 | KEES[166.9984]EES[166.9984]D | −3.84 |
| | OS = Homo sapiens GN = RPLP2 PE = 1 | DDMGFGLFD | |
| | SV = 1 | | |
| ROA3_HUMAN | Heterogeneous nuclear | SSGS[166.9984]PYGGGYGSGGG | 1.53 |
| | ribonucleoprotein A3 - Homo sapiens | SGGYGSR | |
| | (Human) | | |
| SDPR_HUMAN | Serum deprivation-response | EELPDENKSLEETLHT[181.014]V | 1.39 |
| | protein OS = Homo sapiens | DLS[166.9984]SDDDLPHDEEALE | |
| | GN = SDPR PE = 1 SV = 3 | DSAEEKVEESR | |
| SDPR_HUMAN | Serum deprivation-response | IS[166.9984]S[166.9984]GKS[1 | 1.42 |
| | protein OS = Homo sapiens | 66.9984]SPFKVSPLTFGR | |
| | GN = SDPR PE = 1 SV = 3 | | |
| SDPR_HUMAN | Serum deprivation-response | ISSGKS[166.9984]S[166.9984]P | 1.26 |
| | protein OS = Homo sapiens | FKVS[166.9984]PLTFGR | |
| | GN = SDPR PE = 1 SV = 3 | | |
| SDPR_HUMAN | Serum deprivation-response | S[166.9984]SPFKVS[166.9984]P | 1.04 |
| | protein OS = Homo sapiens | LTFGR | |
| | GN = SDPR PE = 1 SV = 3 | | |
| SDPR_HUMAN | Serum deprivation-response | SLEETLHTVDLS[166.9984]S[166 | −1.12 |
| | protein OS = Homo sapiens | .9984]DDDLPHDEEALEDS[166.9 | |
| | GN = SDPR PE = 1 SV = 3 | 984]AEEKVEESR | |
| SDPR_HUMAN | Serum deprivation-response | SLEETLHTVDLS[166.9984]S[166 | −1.65 |
| | protein OS = Homo sapiens | .9984]DDDLPHDEEALEDSAEEK | |
| | GN = SDPR PE = 1 SV = 3 | | |
| SDPR_HUMAN | Serum deprivation-response | SLEETLHTVDLS[166.9984]S[166 | 1.06 |
| | protein OS = Homo sapiens | .9984]DDDLPHDEEALEDSAEEKV | |
| | GN = SDPR PE = 1 SV = 3 | EESR | |
| SDPR_HUMAN | Serum deprivation-response | SLEETLHTVDLSS[166.9984]DDD | −1.02 |
| | protein OS = Homo sapiens | LPHDEEALEDS[166.9984]AEEKV | |
| | GN = SDPR PE = 1 SV = 3 | EESR | |
| SDPR_HUMAN | Serum deprivation-response | SSPFKVS[166.9984]PLTFGR | 1.46 |
| | protein OS = Homo sapiens | | |
| | GN = SDPR PE = 1 SV = 3 | | |
| SDPR_HUMAN | Serum deprivation-response | VS[166.9984]PLTFGR | 1.45 |
| | protein OS = Homo sapiens | | |
| | GN = SDPR PE = 1 SV = 3 | | |
| SRBS2_HUMAN | Sorbin and SH3 domain-containing | DAS[166.9984]SPVPPPHVPPPV | 2.26 |
| | protein 2 OS = Homo sapiens | PPLRPR | |
| | GN = SORBS2 PE = 1 SV = 3 | | |
| SRBS2_HUMAN | Sorbin and SH3 domain-containing | DASS[166.9984]PVPPPHVPPPV | 2.64 |
| | protein 2 OS = Homo sapiens | PPLRPR | |
| | GN = SORBS2 PE = 1 SV = 3 | | |
| SRBS2_HUMAN | Sorbin and SH3 domain-containing | GAEDYPDPPIPHS[166.9984]YSS | −2.16 |
| | protein 2 OS = Homo sapiens | DR | |
| | GN = SORBS2 PE = 1 SV = 3 | | |
| SRBS2_HUMAN | Sorbin and SH3 domain-containing | RKS[166.9984]EPAVGPPR | 1.77 |
| | protein 2 OS = Homo sapiens | | |
| | GN = SORBS2 PE = 1 SV = 3 | | |
| SRBS2_HUMAN | Sorbin and SH3 domain-containing | S[166.9984]EPAVGPPR | 2.67 |
| | protein 2 OS = Homo sapiens | | |
| | GN = SORBS2 PE = 1 SV = 3 | | |
| SRBS2_HUMAN | Sorbin and SH3 domain-containing | SFTSSS[166.9984]PS[166.9984] | 5.55 |
| | protein 2 OS = Homo sapiens | SPSR | |
| | GN = SORBS2 PE = 1 SV = 3 | | |
| SRBS2_HUMAN | Sorbin and SH3 domain-containing | SFTSSSPS[166.9984]SPSR | 1.86 |
| | protein 2 OS = Homo sapiens | | |
| | GN = SORBS2 PE = 1 SV = 3 | | |
| SRBS2_HUMAN | Sorbin and SH3 domain-containing | SHS[166.9984]DNSPNAFK | −1.20 |
| | protein 2 OS = Homo sapiens | | |
| | GN = SORBS2 PE = 1 SV = 3 | | |
| SRBS2_HUMAN | Sorbin and SH3 domain-containing | T[181.014]SPGRVDLPGSSTTLTK | 3.15 |
| | protein 2 OS = Homo sapiens | | |
| | GN = SORBS2 PE = 1 SV = 3 | | |
| SRBS2_HUMAN | Sorbin and SH3 domain-containing | TSPGRVDLPGS[166.9984]STTLT | 3.70 |
| | protein 2 OS = Homo sapiens | K | |
| | GN = SORBS2 PE = 1 SV = 3 | | |
| SRCH_HUMAN | Sarcoplasmic reticulum histidine- | AEVGAPLS[166.9984]PDHS[166 | 1.81 |
| | rich calcium-binding protein | .9984]EEEEEEEEGLEEDEPR | |
| | OS = Homo sapiens GN = HRC PE = 2 | | |
| | SV = 1 | | |
| SRCH_HUMAN | Sarcoplasmic reticulum histidine- | AEVGAPLS[166.9984]PDHSEEEE | −1.16 |
| | rich calcium-binding protein | EEEEGLEEDEPR | |
| | OS = Homo sapiens GN = HRC PE = 2 | | |
| | SV = 1 | | |
| SRCH_HUMAN | Sarcoplasmic reticulum histidine- | AEVGAPLSPDHS[166.9984]EEEE | −1.26 |
| | rich calcium-binding protein | EEEEGLEEDEPR | |
| | OS = Homo sapiens GN = HRC PE = 2 | | |
| | SV = 1 | | |
| SRCH_HUMAN | Sarcoplasmic reticulum histidine- | DDSEEEKEKEEDPGS[166.9984] | −1.32 |
| | rich calcium-binding protein | HEEDDESSEQGEK | |
| | OS = Homo sapiens GN = HRC PE = 2 | | |
| | SV = 1 | | |
| SRCH_HUMAN | Sarcoplasmic reticulum histidine- | DEEEDEDVS[166.9984]TER | −1.30 |
| | rich calcium-binding protein | | |
| | OS = Homo sapiens GN = HRC PE = 2 | | |
| | SV = 1 | | |
| SRCH_HUMAN | Sarcoplasmic reticulum histidine- | EEAGGASS[166.9984]EEESGEDT | −1.37 |
| | rich calcium-binding protein | GPQDAQEYGNYQPGSLC[160.03 | |
| | OS = Homo sapiens GN = HRC PE = 2 | 07]GYC[160.0307]SFC[160.030 | |
| | SV = 1 | 7]NR | |
| SRCH_HUMAN | Sarcoplasmic reticulum histidine- | EEDEEVS[166.9984]AELGHQAP | −2.20 |
| | rich calcium-binding protein | SHR | |
| | OS = Homo sapiens GN = HRC PE = 2 | | |
| | SV = 1 | | |
| SRCH_HUMAN | Sarcoplasmic reticulum histidine- | EKEEDPGS[166.9984]HEEDDESS | −3.18 |
| | rich calcium-binding protein | EQGEK | |
| | OS = Homo sapiens GN = HRC PE = 2 | | |
| | SV = 1 | | |
| SRCH_HUMAN | Sarcoplasmic reticulum histidine- | GHDGEDDEGEEEEEEEEEEEEAS | −2.57 |
| | rich calcium-binding protein | [166.9984]TEYGHQAHR | |
| | OS = Homo sapiens GN = HRC PE = 2 | | |
| | SV = 1 | | |
| SRCH_HUMAN | Sarcoplasmic reticulum histidine- | GHGS[166.9984]EEDEDVSDGH | −1.73 |
| | rich calcium-binding protein | HHHGPSHR | |
| | OS = Homo sapiens GN = HRC PE = 2 | | |
| | SV = 1 | | |
| SRCH_HUMAN | Sarcoplasmic reticulum histidine- | GHGSEDT[181.014]EDSAEHR | −1.66 |
| | rich calcium-binding protein | | |
| | OS = Homo sapiens GN = HRC PE = 2 | | |
| | SV = 1 | | |
| SRCH_HUMAN | Sarcoplasmic reticulum histidine- | GHGSEDTEDS[166.9984]AEHR | −1.28 |
| | rich calcium-binding protein | | |
| | OS = Homo sapiens GN = HRC PE = 2 | | |
| | SV = 1 | | |
| SRCH_HUMAN | Sarcoplasmic reticulum histidine- | GHKS[166.9984]DEEDFQDEYK | 1.23 |
| | rich calcium-binding protein | | |
| | OS = Homo sapiens GN = HRC PE = 2 | | |
| | SV = 1 | | |
| SRCH_HUMAN | Sarcoplasmic reticulum histidine- | HQGHEEDDDDDDDDDDDDDDD | −1.88 |
| | rich calcium-binding protein | DVS[166.9984]IEYR | |
| | OS = Homo sapiens GN = HRC PE = 2 | | |
| | SV = 1 | | |
| SRCH_HUMAN | Sarcoplasmic reticulum histidine- | HQGHRDEEEDEDVS[166.9984]T | −1.07 |
| | rich calcium-binding protein | ER | |
| | OS = Homo sapiens GN = HRC PE = 2 | | |
| | SV = 1 | | |
| SRCH_HUMAN | Sarcoplasmic reticulum histidine- | HRS[166.9984]HEEDDNDDDDV | −1.04 |
| | rich calcium-binding protein | S[166.9984]TEYGHQAHR | |
| | OS = Homo sapiens GN = HRC PE = 2 | | |
| | SV = 1 | | |
| SRCH_HUMAN | Sarcoplasmic reticulum histidine- | HRS[166.9984]HEEDDNDDDDV | −1.92 |
| | rich calcium-binding protein | STEYGHQAHR | |
| | OS = Homo sapiens GN = HRC PE = 2 | | |
| | SV = 1 | | |
| SRCH_HUMAN | Sarcoplasmic reticulum histidine- | SHEEDDNDDDDVS[166.9984]TE | −1.11 |
| | rich calcium-binding protein | YGHQAHR | |
| | OS = Homo sapiens GN = HRC PE = 2 | | |
| | SV = 1 | | |
| SRCH_HUMAN | Sarcoplasmic reticulum histidine- | VGDEGVS[166.9984]GEEVFAEH | −1.31 |
| | rich calcium-binding protein | GGQAR | |
| | OS = Homo sapiens GN = HRC PE = 2 | | |
| | SV = 1 | | |
| SRCH_HUMAN | Sarcoplasmic reticulum histidine- | VPREEDEEVS[166.9984]AELGH | 1.18 |
| | rich calcium-binding protein | QAPSHR | |
| | OS = Homo sapiens GN = HRC PE = 2 | | |
| | SV = 1 | | |
| TEBP_HUMAN | Prostaglandin E synthase 3 - | DWEDDS[166.9984]DEDMSNFD | −2.65 |
| | Homo sapiens (Human) | R | |
| TEBP_HUMAN | Prostaglandin E synthase 3 - | LNWLSVDFNNWKDWEDDS[166 | 1.60 |
| | Homo sapiens (Human) | .9984]DEDM[147.0354]SNFDR | |
| TEBP_HUMAN | Prostaglandin E synthase 3 - | LNWLSVDFNNWKDWEDDS[166 | −2.56 |
| | Homo sapiens (Human) | .9984]DEDMSNFDR | |
| TELT_HUMAN | Telethonin OS = Homo sapiens | EEREDT[181.014]PIQLQELLALET | 1.26 |
| | GN = TCAP PE = 1 SV = 1 | ALGGQC[160.0307]VDR | |
| TELT_HUMAN | Telethonin OS = Homo sapiens | SMS[166.9984]QEAQRG | −1.36 |
| | GN = TCAP PE = 1 SV = 1 | | |
| TNNC1_HUMAN | Troponin C, slow skeletal and | GKS[166.9984]EEELSDLFR | 1.09 |
| | cardiac muscles OS = Homo sapiens | | |
| | GN = TNNC1 PE = 1 SV = 1 | | |
| TNNI3_HUMAN | Troponin I, cardiac muscle | RRS[166.9984]S[166.9984]NYR | −1.56 |
| | OS = Homo sapiens GN = TNNI3 PE = 1 | | |
| | SV = 3 | | |
| TNNT2_HUMAN | Troponin T, cardiac muscle | ELWQS[166.9984]IYNLEAEKFDL | −1.41 |
| | OS = Homo sapiens GN = TNNT2 PE = 1 | QEK | |
| | SV = 3 | | |
| TPIS_HUMAN | Triosephosphate isomerase - | IIYGGS[166.9984]VTGATC[160.0 | −1.24 |
| | Homo sapiens (Human) | 307]K | |
| TPIS_HUMAN | Triosephosphate isomerase - | KQS[166.9984]LGELIGTLNAAK | 2.69 |
| | Homo sapiens (Human) | | |
| TPM1_HUMAN | Tropomyosin alpha-1 chain - | KLVIIES[166.9984]DLER | 1.14 |
| | Homo sapiens (Human) | | |
| TPM1_HUMAN | Tropomyosin alpha-1 chain - | LVIIES[166.9984]DLERAEER | 1.10 |
| | Homo sapiens (Human) | | |
| TPM1_HUMAN | Tropomyosin alpha-1 chain - | S[166.9984]IDDLEDELYAQK | −1.23 |
| | Homo sapiens (Human) | | |
| TPM1_HUMAN | Tropomyosin alpha-1 chain | AISEELDHALNDM[147.0354]TS | 2.39 |
| | OS = Homo sapiens GN = TPM1 PE = 1 | [166.9984]I | |
| | SV = 2 | | |
| TPM1_HUMAN | Tropomyosin alpha-1 chain | AISEELDHALNDMTS[166.9984]I | −1.89 |
| | OS = Homo sapiens GN = TPM1 PE = 1 | | |
| | SV = 2 | | |
| TPM1_HUMAN | Tropomyosin alpha-1 chain | ATDAEADVAS[166.9984]LNRR | 1.45 |
| | OS = Homo sapiens GN = TPM1 PE = 1 | | |
| | SV = 2 | | |
| TPM1_HUMAN | Tropomyosin alpha-1 chain | KATDAEADVAS[166.9984]LNR | 1.33 |
| | OS = Homo sapiens GN = TPM1 PE = 1 | | |
| | SV = 2 | | |
| TPM1_HUMAN | Tropomyosin alpha-1 chain | KATDAEADVAS[166.9984]LNRR | 1.49 |
| | OS = Homo sapiens GN = TPM1 PE = 1 | | |
| | SV = 2 | | |
| TPM1_HUMAN | Tropomyosin alpha-1 chain | LAT[181.014]ALQK | −1.32 |
| | OS = Homo sapiens GN = TPM1 PE = 1 | | |
| | SV = 2 | | |
| TPM2_HUMAN | Tropomyosin beta chain - Homo sapiens | AISEELDNALNDITS[166.9984]L | 1.12 |
| | (Human) | | |
| TPPP_HUMAN | Tubulin polymerization-promoting | AANRT[181.014]PPKSPGDPSK | 1.05 |
| | protein OS = Homo sapiens GN = TPPP | | |
| | PE = 1 SV = 1 | | |
| TPPP_HUMAN | Tubulin polymerization-promoting | AISS[166.9984]PTVSR | 3.26 |
| | protein OS = Homo sapiens GN = TPPP | | |
| | PE = 1 SV = 1 | | |
| VDAC1_HUMAN | Voltage-dependent anion-selective | VNNS[166.9984]SLIGLGYTQTLK | −1.30 |
| | channel protein 1 - Homo sapiens | PGIK | |
| | (Human) | | |
| VDAC2_HUMAN | Voltage-dependent anion-selective | LTFDTTFSPNT[181.014]GK | −1.12 |
| | channel protein 2 - Homo sapiens | | |
| | (Human) | | |
| VDAC2_HUMAN | Voltage-dependent anion-selective | VNNS[166.9984]SLIGVGYTQTLR | −1.30 |
| | channel protein 2 - Homo sapiens | PGVK | |
| | (Human) | | |
| VIME_HUMAN | Vimentin - Homo sapiens (Human) | LRS[166.9984]SVPGVR | −1.31 |
| VIME_HUMAN | Vimentin - Homo sapiens (Human) | TYS[166.9984]LGSALRPSTSR | 1.07 |

| | NIF v IF | IF v NF | IF v NF | NIF v NF | NIF v NF |
|---|---|---|---|---|---|
| Primary Protein | p-value | Fold | p-value | Fold | p-value |
| Name | (ANOVA) | Change | (ANOVA) | Change | (ANOVA) |
| ALBU_HUMAN | 0.7080 | 2.34 | 0.0000 | 2.99 | 0.0000 |
| ALDOA_HUMAN | 0.1240 | 1.00 | 0.8230 | −1.85 | 0.1720 |
| ALDOA_HUMAN | 0.5020 | 1.02 | 0.8530 | −1.35 | 0.5870 |
| ALDOA_HUMAN | 0.9020 | −1.85 | 0.5880 | −1.29 | 0.5980 |
| ANT3_HUMAN | 0.2890 | 2.77 | 0.1150 | 1.98 | 0.5600 |
| ANT3_HUMAN | 0.3350 | 2.50 | 0.2080 | 1.66 | 0.6580 |
| BASI_HUMAN | 0.0000 | 4.62 | 0.0005 | −1.57 | 0.6750 |
| BASI_HUMAN | 0.6110 | −1.66 | 0.2240 | −1.31 | 0.7280 |
| BASI_HUMAN | 0.0002 | 2.48 | 0.0590 | −2.16 | 0.5710 |
| BASI_HUMAN | 0.0360 | 2.56 | 0.0940 | −1.70 | 0.8570 |
| CALD1_HUMAN | 0.6360 | −5.25 | 0.0160 | −2.93 | 0.0190 |
| CALD1_HUMAN | 0.3980 | −1.28 | 0.6030 | 1.26 | 0.9720 |
| CAPZB_HUMAN | 0.0060 | 1.27 | 0.7640 | −1.76 | 0.1420 |
| CASQ2_HUMAN | 0.2410 | −1.55 | 0.4020 | −3.84 | 0.0210 |
| CRIP2_HUMAN | 0.0005 | 1.36 | 0.6380 | −1.19 | 0.6820 |
| CRYAB_HUMAN | 0.6340 | 1.11 | 0.9830 | −1.48 | 0.7400 |
| CSPG2_HUMAN | 0.1060 | −1.05 | 0.9060 | 2.20 | 0.3630 |
| CSRP3_HUMAN | 0.3070 | 1.02 | 0.9980 | −1.62 | 0.2860 |
| CSRP3_HUMAN | 0.3370 | −1.04 | 0.9960 | −1.29 | 0.3420 |
| CSRP3_HUMAN | 0.7780 | −1.05 | 0.8450 | −1.18 | 0.6620 |
| CSRP3_HUMAN | 0.9870 | −1.10 | 0.7040 | −1.23 | 0.7650 |
| CYC_HUMAN | 0.9360 | −1.41 | 0.6460 | −1.35 | 0.8250 |
| CYC_HUMAN | 0.1710 | −1.84 | 0.1340 | −1.03 | 0.9830 |
| CYC_HUMAN | 0.4000 | −2.60 | 0.0020 | −1.95 | 0.7030 |
| DESM_HUMAN | 0.2480 | −1.18 | 0.8320 | 1.27 | 0.6180 |
| DESM_HUMAN | 0.3460 | −1.25 | 0.9690 | 1.72 | 0.1530 |
| DESM_HUMAN | 0.7730 | 1.82 | 0.3600 | 2.55 | 0.0620 |
| DSG2_HUMAN | 0.5550 | −2.81 | 0.1770 | −1.58 | 0.6380 |
| DSG2_HUMAN | 0.1710 | −2.29 | 0.2130 | 1.02 | 0.8730 |
| EF1B_HUMAN | 0.9060 | −1.68 | 0.3300 | −1.56 | 0.4000 |
| FBN1_HUMAN | 0.0007 | −2.39 | 0.0730 | 1.59 | 0.6420 |
| FETUA_HUMAN | 0.3720 | 3.48 | 0.1310 | 1.60 | 0.7390 |
| FETUA_HUMAN | 0.3930 | 4.22 | 0.0310 | 2.02 | 0.5390 |
| FETUA_HUMAN | 0.9200 | 2.14 | 0.3500 | 5.15 | 0.0590 |
| FETUA_HUMAN | 0.7770 | 20.09 | 0.0000 | 8.67 | 0.0000 |
| FETUA_HUMAN | 0.7590 | 7.95 | 0.0007 | 4.24 | 0.1060 |
| FHL2_HUMAN | 0.0130 | −1.04 | 0.9210 | −3.49 | 0.0350 |
| FRIH_HUMAN | 0.7100 | −1.17 | 0.9590 | −1.63 | 0.6690 |
| G3P_HUMAN | 0.6330 | −1.35 | 0.7670 | −1.79 | 0.1420 |
| G3P_HUMAN | 0.8010 | 1.17 | 0.8120 | −1.09 | 0.9510 |
| G3P_HUMAN | 0.5330 | −1.61 | 0.9160 | −1.86 | 0.3240 |
| G3P_HUMAN | 0.6670 | 1.78 | 0.4930 | 1.14 | 0.8970 |
| H12_HUMAN | 0.8560 | −1.56 | 0.6000 | −1.20 | 0.7650 |
| H31_HUMAN | 0.7990 | −2.13 | 0.2930 | −1.53 | 0.6540 |
| HNRPD_HUMAN | 0.1530 | −1.43 | 0.4310 | 1.30 | 0.6690 |
| HNRPD_HUMAN | 0.5170 | −1.28 | 0.9410 | 1.25 | 0.0980 |
| HP1B3_HUMAN | 0.5790 | −1.52 | 0.4310 | −1.05 | 0.9020 |
| HS90A_HUMAN | 0.0002 | 3.74 | 0.0000 | 1.09 | 0.9040 |
| HS90A_HUMAN | 0.7050 | 1.80 | 0.0860 | 1.65 | 0.3240 |
| HS90A_HUMAN | 0.9840 | 1.24 | 0.6810 | 1.33 | 0.5270 |
| HS90B_HUMAN | 0.0850 | 2.76 | 0.4340 | 7.48 | 0.0060 |
| HS90B_HUMAN | 0.9070 | −1.03 | 0.9940 | 1.25 | 0.8900 |
| HS90B_HUMAN | 0.4560 | −1.27 | 0.8130 | 1.22 | 0.7200 |
| HSPB1_HUMAN | 0.0001 | 1.76 | 0.4800 | −2.28 | 0.0720 |
| HSPB1_HUMAN | 0.0000 | −1.07 | 0.8020 | −3.56 | 0.0001 |
| HSPB1_HUMAN | 0.3570 | 4.53 | 0.0008 | 1.14 | 0.6120 |
| HSPB7_HUMAN | 0.0880 | 3.83 | 0.0520 | 1.36 | 0.8400 |
| HSPB7_HUMAN | 0.1410 | 5.03 | 0.1040 | 1.63 | 0.8280 |
| HSPB7_HUMAN | 0.0060 | 2.83 | 0.0510 | −1.22 | 0.8170 |
| HSPB7_HUMAN | 0.1480 | 2.51 | 0.3550 | −1.27 | 0.8810 |
| HSPB7_HUMAN | 0.0020 | 2.27 | 0.1730 | −1.13 | 0.6930 |
| ICAL_HUMAN | 0.9200 | −1.86 | 0.2940 | −2.00 | 0.6620 |
| ICAL_HUMAN | 0.9710 | −1.63 | 0.3610 | −1.89 | 0.6700 |
| ICAL_HUMAN | 0.6660 | −1.54 | 0.4360 | −1.28 | 0.9820 |
| KAD1_HUMAN | 0.6710 | −1.61 | 0.5990 | −1.10 | 0.9040 |
| KAD1_HUMAN | 0.9370 | −1.01 | 0.9600 | −1.08 | 0.9620 |
| KAD1_HUMAN | 0.5740 | −2.00 | 0.3110 | −1.27 | 0.7430 |
| KCRM_HUMAN | 0.9370 | −2.11 | 0.3880 | −2.52 | 0.3830 |
| KCRM_HUMAN | 0.0370 | −1.79 | 0.5830 | −3.77 | 0.0001 |
| KCRM_HUMAN | 0.8530 | −2.04 | 0.2720 | −2.52 | 0.0590 |
| KCRM_HUMAN | 0.4620 | −3.67 | 0.0050 | −5.20 | 0.0000 |
| KCRS_HUMAN | 0.9440 | −2.28 | 0.6810 | −1.76 | 0.4600 |
| KNG1_HUMAN | 0.1440 | 2.52 | 0.0010 | 1.93 | 0.0620 |
| KNG1_HUMAN | 0.9610 | 2.47 | 0.0530 | 2.36 | 0.1370 |
| KNG1_HUMAN | 0.6260 | 4.07 | 0.0000 | 2.93 | 0.0240 |
| KNG1_HUMAN | 0.7000 | 2.16 | 0.0080 | 1.96 | 0.0540 |
| LU_HUMAN | 0.9610 | −1.00 | 0.8440 | 1.18 | 0.8770 |
| LU_HUMAN | 0.1670 | 1.23 | 0.9600 | −2.24 | 0.4280 |
| MARCS_HUMAN | 0.4490 | 1.75 | 0.2360 | 3.23 | 0.0020 |
| MARCS_HUMAN | 0.0000 | 1.62 | 0.0290 | 2.61 | 0.1760 |
| MARCS_HUMAN | 0.3080 | 1.39 | 0.6260 | −1.43 | 0.7510 |
| MARCS_HUMAN | 0.4490 | 2.66 | 0.0610 | 1.23 | 0.7710 |
| MARCS_HUMAN | 0.3190 | −1.00 | 0.8580 | 2.15 | 0.0440 |
| MARCS_HUMAN | 0.2410 | −1.27 | 0.7430 | 1.50 | 0.2940 |
| MLRV_HUMAN | 0.3420 | −1.86 | 0.7450 | 1.53 | 0.4090 |
| MLRV_HUMAN | 0.0060 | 1.70 | 0.7800 | −2.08 | 0.0880 |
| MLRV_HUMAN | 0.2020 | −1.98 | 0.5540 | 1.52 | 0.7030 |
| MLRV_HUMAN | 0.3480 | 1.88 | 0.5550 | −1.22 | 0.8800 |
| MYOZ2_HUMAN | 0.9500 | 1.55 | 0.1770 | 1.23 | 0.4280 |
| MYOZ2_HUMAN | 0.5420 | −1.48 | 0.2420 | −1.23 | 0.4520 |
| MYOZ2_HUMAN | 0.8500 | −1.22 | 0.9980 | 1.31 | 0.8240 |
| MYOZ2_HUMAN | 0.1820 | 3.02 | 0.0003 | 1.97 | 0.0030 |
| MYOZ2_HUMAN | 0.9810 | −1.24 | 0.4450 | −1.28 | 0.2190 |
| MYPT1_HUMAN | 0.7790 | 1.21 | 0.9060 | 1.46 | 0.6730 |
| MYPT1_HUMAN | 0.7030 | 1.31 | 0.6540 | 1.84 | 0.1880 |
| MYPT1_HUMAN | 0.0260 | 1.25 | 0.6460 | −2.15 | 0.1140 |
| MYPT1_HUMAN | 0.3180 | 1.20 | 0.8230 | −1.60 | 0.4280 |
| MYPT1_HUMAN | 0.8880 | 1.28 | 0.7520 | 1.25 | 0.8130 |
| MYPT2_HUMAN | 0.5910 | −3.41 | 0.1660 | −1.52 | 0.3420 |
| MYPT2_HUMAN | 0.1120 | −1.37 | 0.6460 | −3.03 | 0.0040 |
| NCAM1_HUMAN | 0.9040 | −1.66 | 0.5830 | −1.81 | 0.3900 |
| NEBL_HUMAN | 0.7070 | 1.75 | 0.6460 | 2.78 | 0.1760 |
| NEXN_HUMAN | 0.1450 | −5.85 | 0.1760 | −1.05 | 0.7240 |
| NEXN_HUMAN | 0.0005 | −16.05 | 0.0003 | −1.19 | 0.6860 |
| NEXN_HUMAN | 0.1370 | −1.15 | 0.9240 | 2.42 | 0.0020 |
| NP1L4_HUMAN | 0.5680 | 1.44 | 0.3880 | 1.28 | 0.5850 |
| NP1L4_HUMAN | 0.8780 | 1.36 | 0.7670 | −1.07 | 0.9560 |
| OCAD1_HUMAN | 0.0140 | −3.33 | 0.0510 | −1.00 | 0.9120 |
| ODPA_HUMAN | 0.0000 | −61.57 | 0.0000 | 2.00 | 0.7030 |
| ODPA_HUMAN | 0.0000 | −32.49 | 0.0000 | −1.00 | 0.9390 |
| ODPA_HUMAN | 0.0000 | −27.30 | 0.0000 | −1.06 | 0.9110 |
| ODPA_HUMAN | 0.5740 | −1.15 | 0.6460 | 1.01 | 0.9390 |
| ODPA_HUMAN | 0.5910 | 1.89 | 0.6400 | −1.17 | 0.9490 |
| ODPA_HUMAN | 0.9040 | 1.56 | 0.8440 | −1.05 | 0.9680 |
| PEBP1_HUMAN | 0.9980 | −1.14 | 0.7420 | −1.14 | 0.7340 |
| PEBP1_HUMAN | 0.9790 | −1.96 | 0.3180 | −1.99 | 0.1520 |
| PGRC2_HUMAN | 0.1580 | −1.22 | 0.4020 | 1.08 | 0.9270 |
| PGRC2_HUMAN | 0.8670 | 1.25 | 0.8010 | 1.07 | 0.9500 |
| PGRC2_HUMAN | 0.4490 | 1.46 | 0.6270 | −1.05 | 0.9040 |
| PGRC2_HUMAN | 0.4140 | −1.13 | 0.5910 | 1.02 | 0.9820 |
| POPD1_HUMAN | 0.0120 | 2.03 | 0.9410 | −3.96 | 0.1710 |
| POPD1_HUMAN | 0.9360 | −2.65 | 0.7520 | −1.58 | 0.7490 |
| POPD1_HUMAN | 0.0840 | 1.35 | 0.7040 | −4.40 | 0.1280 |
| POPD1_HUMAN | 0.5440 | −2.02 | 0.7040 | 1.28 | 0.8880 |
| POPD1_HUMAN | 0.7770 | 1.37 | 0.9220 | −1.24 | 0.9130 |
| POPD1_HUMAN | 0.0000 | 1.30 | 0.9980 | −7.72 | 0.0000 |
| POPD1_HUMAN | 0.5750 | −2.23 | 0.0800 | −1.40 | 0.1630 |
| POPD1_HUMAN | 0.5580 | −1.71 | 0.3880 | −1.21 | 0.7170 |
| PTRF_HUMAN | 0.9990 | 1.12 | 0.8590 | −1.00 | 0.9020 |
| PTRF_HUMAN | 0.9840 | −1.03 | 0.9740 | −1.06 | 0.9990 |
| PTRF_HUMAN | 0.2220 | −2.38 | 0.2260 | −1.11 | 0.9380 |
| PTRF_HUMAN | 0.1300 | −1.18 | 0.6540 | 1.08 | 0.8640 |
| PTRF_HUMAN | 0.5180 | −1.10 | 0.9190 | 1.38 | 0.5790 |
| PTRF_HUMAN | 0.8880 | 1.54 | 0.2700 | 1.67 | 0.1130 |
| PTRF_HUMAN | 0.8150 | 1.39 | 0.7030 | 1.11 | 0.9070 |
| PTRF_HUMAN | 0.6310 | −2.22 | 0.6460 | 1.03 | 0.9230 |
| QCR6_HUMAN | 0.8780 | −1.30 | 0.8180 | −1.80 | 0.6930 |
| QCR6_HUMAN | 0.9800 | −1.81 | 0.4630 | −1.95 | 0.7030 |
| RLA2_HUMAN | 0.5310 | −5.06 | 0.3020 | −1.42 | 0.8470 |
| RLA2_HUMAN | 0.1110 | 1.35 | 0.9350 | −2.84 | 0.3780 |
| ROA3_HUMAN | 0.4870 | −1.19 | 0.7960 | 1.29 | 0.7370 |
| SDPR_HUMAN | 0.9830 | 1.21 | 0.2720 | 1.68 | 0.1900 |
| SDPR_HUMAN | 0.8340 | −1.22 | 0.9180 | 1.16 | 0.9040 |
| SDPR_HUMAN | 0.9990 | 1.01 | 0.8930 | 1.27 | 0.8700 |
| SDPR_HUMAN | 0.9200 | 1.10 | 0.8180 | 1.14 | 0.7650 |
| SDPR_HUMAN | 0.9060 | 1.21 | 0.6270 | 1.08 | 0.8250 |
| SDPR_HUMAN | 0.6310 | 1.40 | 0.3600 | −1.17 | 0.9930 |
| SDPR_HUMAN | 0.9250 | 1.31 | 0.3170 | 1.39 | 0.2930 |
| SDPR_HUMAN | 0.9500 | −1.12 | 0.9980 | −1.13 | 0.9430 |
| SDPR_HUMAN | 0.5790 | −2.42 | 0.0820 | −1.66 | 0.5700 |
| SDPR_HUMAN | 0.6530 | −1.21 | 0.8610 | 1.20 | 0.7290 |
| SRBS2_HUMAN | 0.0040 | 1.52 | 0.3420 | 3.44 | 0.0000 |
| SRBS2_HUMAN | 0.0140 | 2.64 | 0.2380 | 6.98 | 0.0007 |
| SRBS2_HUMAN | 0.1930 | 3.77 | 0.0003 | 1.75 | 0.3710 |
| SRBS2_HUMAN | 0.0260 | 1.25 | 0.8020 | 2.21 | 0.0360 |
| SRBS2_HUMAN | 0.3260 | 2.68 | 0.4370 | 7.16 | 0.0180 |
| SRBS2_HUMAN | 0.0000 | −1.69 | 0.2940 | 3.28 | 0.0350 |
| SRBS2_HUMAN | 0.0810 | 1.39 | 0.3840 | 2.60 | 0.0020 |
| SRBS2_HUMAN | 0.9200 | 1.25 | 0.8930 | 1.04 | 0.9820 |
| SRBS2_HUMAN | 0.0010 | −1.07 | 0.8410 | 2.95 | 0.0060 |
| SRBS2_HUMAN | 0.0000 | 1.04 | 0.9970 | 3.85 | 0.0004 |
| SRCH_HUMAN | 0.2230 | −1.65 | 0.3710 | 1.10 | 0.7700 |
| SRCH_HUMAN | 0.5740 | −1.00 | 0.8520 | −1.16 | 0.3890 |
| SRCH_HUMAN | 0.4420 | 1.09 | 0.9780 | −1.16 | 0.4490 |
| SRCH_HUMAN | 0.6670 | −1.02 | 0.9590 | −1.34 | 0.7030 |
| SRCH_HUMAN | 0.7960 | −1.11 | 0.9190 | −1.44 | 0.6630 |
| SRCH_HUMAN | 0.4430 | −1.20 | 0.8120 | −1.64 | 0.2200 |
| SRCH_HUMAN | 0.0700 | 1.20 | 0.9980 | −1.84 | 0.1130 |
| SRCH_HUMAN | 0.0040 | 1.65 | 0.4850 | −1.92 | 0.3070 |
| SRCH_HUMAN | 0.0280 | −1.36 | 0.6690 | −3.50 | 0.0040 |
| SRCH_HUMAN | 0.5800 | 1.09 | 0.6290 | −1.58 | 0.3670 |
| SRCH_HUMAN | 0.9990 | −2.62 | 0.0000 | −4.36 | 0.0000 |
| SRCH_HUMAN | 0.7460 | −2.61 | 0.6460 | −3.34 | 0.1870 |
| SRCH_HUMAN | 0.7950 | −1.04 | 0.8430 | 1.18 | 0.9510 |
| SRCH_HUMAN | 0.1740 | 1.17 | 0.8530 | −1.61 | 0.3250 |
| SRCH_HUMAN | 0.9980 | −1.16 | 0.4660 | −1.25 | 0.6910 |
| SRCH_HUMAN | 0.9400 | 1.68 | 0.7560 | 1.63 | 0.7210 |
| SRCH_HUMAN | 0.4490 | 1.17 | 0.9720 | −1.64 | 0.4280 |
| SRCH_HUMAN | 0.8120 | −1.11 | 0.6430 | −1.24 | 0.5840 |
| SRCH_HUMAN | 0.6320 | −2.26 | 0.0070 | −2.97 | 0.0030 |
| SRCH_HUMAN | 0.1720 | −1.34 | 0.2200 | −1.13 | 0.4850 |
| TEBP_HUMAN | 0.3560 | 1.78 | 0.7970 | −1.49 | 0.7890 |
| TEBP_HUMAN | 0.9200 | −2.24 | 0.7520 | −1.41 | 0.8130 |
| TEBP_HUMAN | 0.1450 | 1.60 | 0.4970 | −1.60 | 0.6860 |
| TELT_HUMAN | 0.6920 | 1.07 | 0.9310 | 1.34 | 0.5860 |
| TELT_HUMAN | 0.9200 | −1.19 | 0.9590 | −1.62 | 0.8940 |
| TNNC1_HUMAN | 0.9580 | −1.85 | 0.5430 | −1.70 | 0.6060 |
| TNNI3_HUMAN | 0.4490 | −1.30 | 0.5490 | −2.02 | 0.1030 |
| TNNT2_HUMAN | 0.7480 | −1.09 | 0.9980 | −1.54 | 0.6800 |
| TPIS_HUMAN | 0.8240 | −1.29 | 0.7430 | −1.60 | 0.3980 |
| TPIS_HUMAN | 0.0490 | −2.17 | 0.2160 | 1.24 | 0.7550 |
| TPM1_HUMAN | 0.9390 | −1.77 | 0.3730 | −1.55 | 0.2830 |
| TPM1_HUMAN | 0.9200 | −1.65 | 0.4350 | −1.51 | 0.3210 |
| TPM1_HUMAN | 0.8540 | −1.47 | 0.4790 | −1.81 | 0.2720 |
| TPM1_HUMAN | 0.2410 | −2.79 | 0.1420 | −1.16 | 0.8130 |
| TPM1_HUMAN | 0.5710 | 1.05 | 0.6890 | −1.80 | 0.5270 |
| TPM1_HUMAN | 0.6820 | −2.24 | 0.2710 | −1.54 | 0.5640 |
| TPM1_HUMAN | 0.7830 | −1.97 | 0.3120 | −1.48 | 0.6630 |
| TPM1_HUMAN | 0.8380 | −2.00 | 0.5880 | −1.34 | 0.7090 |
| TPM1_HUMAN | 0.8750 | −2.27 | 0.3840 | −3.00 | 0.1420 |
| TPM2_HUMAN | 0.7050 | −1.35 | 0.1760 | −1.20 | 0.6930 |
| TPPP_HUMAN | 0.0000 | −2.47 | 0.0000 | −2.36 | 0.1420 |
| TPPP_HUMAN | 0.0300 | −1.71 | 0.4060 | 1.91 | 0.7050 |
| VDAC1_HUMAN | 0.7980 | −1.89 | 0.1600 | −2.44 | 0.0140 |
| VDAC2_HUMAN | 0.9840 | −1.15 | 0.8930 | −1.29 | 0.8670 |
| VDAC2_HUMAN | 0.6780 | −1.87 | 0.1880 | −2.43 | 0.0140 |
| VIME_HUMAN | 0.8830 | 2.31 | 0.6270 | 1.76 | 0.6820 |
| VIME_HUMAN | 0.9840 | 4.16 | 0.0210 | 4.45 | 0.0170 |

1. A method of treating a cardiac disease or disorder and/or improving cardiac function in a subject in need thereof, comprising administering to the subject an effective amount of a substance that modulates the expression of nucleic acid encoding fetuin A in cells of the subject, thereby treating the cardiac disease or disorder and/or reducing fibrosis associated with the cardiac disease or disorder and/or improving cardiac function in the subject.

2-8. (canceled)

9. A method of treating a cardiac disease or disorder and/or improving cardiac function in a subject in need thereof, comprising administering to the subject an effective amount of a nucleic acid molecule that encodes the amino acid sequence of C6ORF142, wherein the amino acid sequence is modified to lack Ser67 phosphorylation, thereby treating the cardiac disease or disorder and/or improving cardiac function in the subject.

10. A method of treating a cardiac disease or disorder and/or improving cardiac function in a subject in need thereof, comprising administering to the subject an effective amount of a substance that modulates the amount and/or activity of C6ORF142 and/or inhibits phosphorylation of Ser67 of C6ORF142, thereby treating the cardiac disease or disorder and/or improving cardiac function in the subject.

11-19. (canceled)


SEQUENCE LISTING (1084 sequences)

SEQ ID NO: 1 - protein, 20 aa, Homo sapiens
  MISC_FEATURE (13)..(13): Phosphyrlation site
HTFMGVVSLGSPSGEVSHPR

SEQ ID NO: 2 - protein, 20 aa, Homo sapiens
  MISC_FEATURE (17)..(17): Phosphyrlation site
HTFMGVVSLGSPSGEVSHPR

SEQ ID NO: 3 - protein, 18 aa, Homo sapiens
  MISC_FEATURE (2)..(2): Phosphyrlation site
ATSNVFAMFDQSQIQEFK

SEQ ID NO: 4 - protein, 16 aa, Homo sapiens
  MISC_FEATURE (2)..(2): Phosphyrlation site
GSPKPSPQPSPKPSPK

SEQ ID NO: 5 - protein, 17 aa, Homo sapiens
  MISC_FEATURE (5)..(5): Phosphyrlation site
EMLASDDEEDVSSKVEK

SEQ ID NO: 6 - protein, 14 aa, Homo sapiens
  MISC_FEATURE (5)..(5): Phosphyrlation site
YHGHSMSDPGVSYR

SEQ ID NO: 7 - protein, 14 aa, Homo sapiens
  MISC_FEATURE (5)..(5): Phosphyrlation site
YHGHSMSDPGVSYR

SEQ ID NO: 8 - protein, 9 aa, Homo sapiens
  MISC_FEATURE (5)..(5): Phosphyrlation site
YGMGTSVER

SEQ ID NO: 9 - protein, 20 aa, Homo sapiens
  MISC_FEATURE (13)..(13): Phosphyrlation site
HTFMGVVSLGSPSGEVSHPR

SEQ ID NO: 10 - protein, 14 aa, Homo sapiens
  MISC_FEATURE (6)..(6): Phosphyrlation site
IEDVGSDEEDDSGK

SEQ ID NO: 11 - protein, 9 aa, Homo sapiens
  MISC_FEATURE (1)..(1): Phosphyrlation site
SEPAVGPPR

SEQ ID NO: 12 - protein, 21 aa, Homo sapiens
  MISC_FEATURE (4)..(4): Phosphyrlation site
DASSPVPPPHVPPPVPPLRPR

SEQ ID NO: 13 - protein, 18 aa, Homo sapiens
  MISC_FEATURE (2)..(2): Phosphyrlation site
NSIASSSDSDDGLHQFLR

SEQ ID NO: 14 - protein, 11 aa, Homo sapiens
LSSQISAGEEK

SEQ ID NO: 15 - protein, 16 aa, Homo sapiens
GSPKPSPQPSPKPSPK

SEQ ID NO: 16 - protein, 9 aa, Homo sapiens
NSLSPATQR

SEQ ID NO: 17 - protein, 16 aa, Homo sapiens
GSPKPSPQPSPKPSPK

SEQ ID NO: 18 - protein, 26 aa, Homo sapiens
KPEDVLDDDDAGSAPLKSSGQHQNDK

SEQ ID NO: 19 - protein, 14 aa, Homo sapiens
KRSSSEDAESLAPR

SEQ ID NO: 20 - protein, 21 aa, Homo sapiens
AGGANSNVFSMFEQTQIQEFK

SEQ ID NO: 21 - protein, 20 aa, Homo sapiens
ESEDKPEIEDVGSDEEEEKK

SEQ ID NO: 22 - protein, 18 aa, Homo sapiens
KSSSISEEKGDSDDEKPR

SEQ ID NO: 23 - protein, 18 aa, Homo sapiens
NSIASSSDSDDGLHQFLR

SEQ ID NO: 24 - protein, 16 aa, Homo sapiens
GTSSMSSLHVSSPHQR

SEQ ID NO: 25 - protein, 7 aa, Homo sapiens
YISFEER

SEQ ID NO: 26 - protein, 15 aa, Homo sapiens
GPSWDPFRDWYPHSR

SEQ ID NO: 27 - protein, 18 aa, Homo sapiens
TDSREDEISPPPPNPVVK

SEQ ID NO: 28 - protein, 15 aa, Homo sapiens
LVSFHDDSDEDLLHI

SEQ ID NO: 29 - protein, 32 aa, Homo sapiens
GHDGEDDEGEEEEEEEEEEEEASTEYGHQAHR

SEQ ID NO: 30 - protein, 21 aa, Homo sapiens
GTGGVDTAAVGSVFDVSNADR

SEQ ID NO: 31 - protein, 12 aa, Homo sapiens
SFTSSSPSSPSR

SEQ ID NO: 32 - protein, 11 aa, Homo sapiens
EGVTPWASFKK

SEQ ID NO: 33 - protein, 17 aa, Homo sapiens
TSPGRVDLPGSSTTLTK

SEQ ID NO: 34 - protein, 17 aa, Homo sapiens
VGSLDNVGHLPAGGAVK

SEQ ID NO: 35 - protein, 17 aa, Homo sapiens
TSPGRVDLPGSSTTLTK

SEQ ID NO: 36 - protein, 11 aa, Homo sapiens
SRSTPPSIAAK

SEQ ID NO: 37 - protein, 12 aa, Homo sapiens
RGESLDNLDSPR

SEQ ID NO: 38 - protein, 21 aa, Homo sapiens
DASSPVPPPHVPPPVPPLRPR

SEQ ID NO: 39 - protein, 14 aa, Homo sapiens
SYSPDGKESPSDKK

SEQ ID NO: 40 - protein, 21 aa, Homo sapiens
DASSPVPPPHVPPPVPPLRPR

SEQ ID NO: 41 - protein, 9 aa, Homo sapiens
YGMGTSVER

SEQ ID NO: 42 - protein, 14 aa, Homo sapiens
YHGHSMSDPGVSYR

SEQ ID NO: 43 - protein, 14 aa, Homo sapiens
YHGHSMSDPGVSYR

SEQ ID NO: 44 - protein, 17 aa, Homo sapiens
EMLASDDEEDVSSKVEK

SEQ ID NO: 45 - protein, 9 aa, Homo sapiens
EIPTPEPLK

SEQ ID NO: 46 - protein, 9 aa, Homo sapiens
NSLSPATQR

SEQ ID NO: 47 - protein, 11 aa, Homo sapiens
DPFRDSPLSSR

SEQ ID NO: 48 - protein, 11 aa, Homo sapiens
RVKSPEPSHPK

SEQ ID NO: 49 - protein, 14 aa, Homo sapiens
AVSPTETKPTPTEK

SEQ ID NO: 50 - protein, 8 aa, Homo sapiens
NASVPNLR

SEQ ID NO: 51 - protein, 31 aa, Homo sapiens
TVAISDAAQLPHDYCTTPGGTLFSTTPGGTR

SEQ ID NO: 52 - protein, 31 aa, Homo sapiens; identical to SEQ ID NO: 51

SEQ ID NO: 53 - protein, 18 aa, Homo sapiens
DLSSSPPGPYGQEMYAFR

SEQ ID NO: 54 - protein, 18 aa, Homo sapiens
DLSSSPPGPYGQEMYAFR

SEQ ID NO: 55 - protein, 19 aa, Homo sapiens
SHNDFVAILDLPEGEHQYK

SEQ ID NO: 56 - protein, 19 aa, Homo sapiens
KLSVPTSDEEDEVPAPKPR

SEQ ID NO: 57 - protein, 19 aa, Homo sapiens
KLSVPTSDEEDEVPAPKPR

SEQ ID NO: 58 - protein, 19 aa, Homo sapiens
KLSVPTSDEEDEVPAPKPR

SEQ ID NO: 59 - protein, 18 aa, Homo sapiens
LSVPTSDEEDEVPAPKPR

SEQ ID NO: 60 - protein, 18 aa, Homo sapiens
LSVPTSDEEDEVPAPKPR

SEQ ID NO: 61 - protein, 14 aa, Homo sapiens
RSSGREEDDEELLR

SEQ ID NO: 62 - protein, 13 aa, Homo sapiens
SSGREEDDEELLR

SEQ ID NO: 63 - protein, 14 aa, Homo sapiens
STSQGSINSPVYSR

SEQ ID NO: 64 - protein, 15 aa, Homo sapiens
TLSPTPSAEGYQDVR

SEQ ID NO: 65 - protein, 19 aa, Homo sapiens
AESPAEKVPEESVLPLVQK

SEQ ID NO: 66 - protein, 9 aa, Homo sapiens
KISVVSATK

SEQ ID NO: 67 - protein, 18 aa, Homo sapiens
KSSSISEEKGDSDDEKPR

SEQ ID NO: 68 - protein, 18 aa, Homo sapiens
KSSSISEEKGDSDDEKPR

SEQ ID NO: 69 - protein, 10 aa, Homo sapiens
RLSQPESAEK

SEQ ID NO: 70 - protein, 17 aa, Homo sapiens
SSSISEEKGDSDDEKPR

SEQ ID NO: 71 - protein, 9 aa, Homo sapiens
TAQVPSPPR

SEQ ID NO: 72 - protein, 20 aa, Homo sapiens
DKKEPVANQDPVSPSLVQGR

SEQ ID NO: 73 - protein, 17 aa, Homo sapiens
EPVANQDPVSPSLVQGR

SEQ ID NO: 74 - protein, 12 aa, Homo sapiens
SFEDLTDHPVTR

SEQ ID NO: 75 - protein, 36 aa, Homo sapiens
GLSQMTTSADTDVDTSKDKTESVTSGPMSPEGSPSK

SEQ ID NO: 76 - protein, 19 aa, Homo sapiens
SSSPGKPQAVSSLNSSHSR

SEQ ID NO: 77 - protein, 9 aa, Homo sapiens
STQSLSLQR

SEQ ID NO: 78 - protein, 10 aa, Homo sapiens
SFLQSLECLR

SEQ ID NO: 79 - protein, 14 aa, Homo sapiens
KGDRSPEPGQTWTR

SEQ ID NO: 80 - protein, 17 aa, Homo sapiens
LKSEDGVEGDLGETQSR

SEQ ID NO: 81 - protein, 17 aa, Homo sapiens
DSEDVSERDSDKEMATK

SEQ ID NO: 82 - protein, 11 aa, Homo sapiens
EGVTPWASFKK

SEQ ID NO: 83 - protein, 13 aa, Homo sapiens
RPSESDKEDELDK

SEQ ID NO: 84 - protein, 15 aa, Homo sapiens
RPSESDKEDELDKVK

SEQ ID NO: 85 - protein, 8 aa, Homo sapiens
SPPSPVER

SEQ ID NO: 86 - protein, 30 aa, Homo sapiens
VKPPPSPTTEGPSLQPDLAPEEAAGTQRPK

SEQ ID NO: 87 - protein, 30 aa, Homo sapiens
AATAARPPAPPPAPQPPSPTPSPPRPTLAR

SEQ ID NO: 88 - protein, 12 aa, Homo sapiens
SLPVSVPVWGFK

SEQ ID NO: 89 - protein, 13 aa, Homo sapiens
TCVADESAENCDK

SEQ ID NO: 90 - protein, 14 aa, Homo sapiens
GILAADESTGSIAK

SEQ ID NO: 91 - protein, 14 aa, Homo sapiens
GILAADESTGSIAK

SEQ ID NO: 92 - protein, 15 aa, Homo sapiens
GILAADESTGSIAKR

SEQ ID NO: 93 - protein, 10 aa, Homo sapiens
EADAATSFLR

SEQ ID NO: 94 - protein, 7 aa, Homo sapiens
KTSLDLR

SEQ ID NO: 95 - protein, 17 aa, Homo sapiens
ATEDEGSEQKIPEATNR

SEQ ID NO: 96 - protein, 18 aa, Homo sapiens
KATEDEGSEQKIPEATNR

SEQ ID NO: 97 - protein, 13 aa, Homo sapiens
DYVSQFEGSALGK

SEQ ID NO: 98 - protein, 26 aa, Homo sapiens
VWTSGQVEEYDLDADDINSRVEMKPK

SEQ ID NO: 99 - protein, 13 aa, Homo sapiens
ASSPPDRIDIFGR

SEQ ID NO: 100 - protein, 20 aa, Homo sapiens
CSHQPISLLGSFLTEESPDK

SEQ ID NO: 101 - protein, 23 aa, Homo sapiens
MQMVPSLPPASECAGEEKRVGTR

SEQ ID NO: 102 - protein, 17 aa, Homo sapiens
VQNAGYSVSRPEGDTGK

SEQ ID NO: 103 - protein, 15 aa, Homo sapiens
SSVQGASSREGSPAR

SEQ ID NO: 104 - protein, 23 aa, Homo sapiens
VPPAPVPCPPPSPGPSAVPSSPK

SEQ ID NO: 105 - protein, 17 aa, Homo sapiens
ELLELDSVETGGQDSVR

SEQ ID NO: 106 - protein, 26 aa, Homo sapiens
KPEDVLDDDDAGSAPLKSSGQHQNDK

SEQ ID NO: 107 - protein, 18 aa, Homo sapiens
RKPEDVLDDDDAGSAPLK

SEQ ID NO: 108 - protein, 27 aa, Homo sapiens
RKPEDVLDDDDAGSAPLKSSGQHQNDK

SEQ ID NO: 109 - protein, 27 aa, Homo sapiens
RKPEDVLDDDDAGSAPLKSSGQHQNDK

SEQ ID NO: 110 - protein, 16 aa, Homo sapiens
FNDSEGDDTEETEDYR

SEQ ID NO: 111 - protein, 9 aa, Homo sapiens
IDISPSTLR

SEQ ID NO: 112 - protein, 13 aa, Homo sapiens
KAEGEPQEESPLK

SEQ ID NO: 113 - protein, 15 aa, Homo sapiens
KAEGEPQEESPLKSK

SEQ ID NO: 114 - protein, 13 aa, Homo sapiens
LKDLFDYSPPLHK

SEQ ID NO: 115 - protein, 10 aa, Homo sapiens
RIDISPSTLR

SEQ ID NO: 116 - protein, 10 aa, Homo sapiens
STFREESPLR

SEQ ID NO: 117 - protein, 15 aa, Homo sapiens
YSPSQNSPIHHIPSR

SEQ ID NO: 118 - protein, 15 aa, Homo sapiens
YSPSQNSPIHHIPSR

SEQ ID NO: 119 - protein, 22 aa, Homo sapiens
DHSSQSEEEVVEGEKEVEALKK

SEQ ID NO: 120 - protein, 13 aa, Homo sapiens
KGSITEYTAAEEK

SEQ ID NO: 121 - protein, 29 aa, Homo sapiens
AGPDLPSLPSHALEDEGWAAAAPSPGSAR

SEQ ID NO: 122 - protein, 25 aa, Homo sapiens
SKSPPKVPIVIQDDSLPAGPPPQIR

SEQ ID NO: 123 - protein, 25 aa, Homo sapiens
SKSPPKVPIVIQDDSLPAGPPPQIR

SEQ ID NO: 124 - protein, 11 aa, Homo sapiens
SSSLDALGPTR

SEQ ID NO: 125 - protein, 16 aa, Homo sapiens
SASQAEEEPSVEPVKK

SEQ ID NO: 126 - protein, 16 aa, Homo sapiens
RGSIGENQVEVMVEEK

SEQ ID NO: 127 - protein, 23 aa, Homo sapiens
TPDGNKSPAPKPSDLRPGDVSSK

SEQ ID NO: 128 - protein, 12 aa, Homo sapiens
AEEDEILNRSPR

SEQ ID NO: 129 - protein, 20 aa, Homo sapiens
SDAEEDGGTVSQEEEDRKPK

SEQ ID NO: 130 - protein, 9 aa, Homo sapiens
ELSQVLTQR

SEQ ID NO: 131 - protein, 18 aa, Homo sapiens
SSIMSITAEPPGNDSIVR

SEQ ID NO: 132 - protein, 16 aa, Homo sapiens
DIGSESTEDQAMEDIK

SEQ ID NO: 133 - protein, 16 aa, Homo sapiens
DIGSESTEDQAMEDIK

SEQ ID NO: 134 - protein, 16 aa, Homo sapiens
DIGSESTEDQAMEDIK

SEQ ID NO: 135 - protein, 16 aa, Homo sapiens
DIGSESTEDQAMEDIK

SEQ ID NO: 136 - protein, 14 aa, Homo sapiens
VPQLEIVPNSAEER

SEQ ID NO: 137 - protein, 16 aa, Homo sapiens
YKVPQLEIVPNSAEER

SEQ ID NO: 138 - protein, 12 aa, Homo sapiens
EQLSTSEENSKK

SEQ ID NO: 139 - protein, 11 aa, Homo sapiens
EQLSTSEENSK

SEQ ID NO: 140 - protein, 17 aa, Homo sapiens
NMAINPSKENLCSTFCK

SEQ ID NO: 141 - protein, 17 aa, Homo sapiens
NMAINPSKENLCSTFCK

SEQ ID NO: 142 - protein, 12 aa, Homo sapiens
TVDMESTEVFTK

SEQ ID NO: 143 - protein, 12 aa, Homo sapiens
TVDMESTEVFTK

SEQ ID NO: 144 - protein, 21 aa, Homo sapiens
KYDLLCLYYHEPVSSDKVTQK

SEQ ID NO: 145 - protein, 25 aa, Homo sapiens
ADVQLFMDDDSYSHHSGLEYADPEK

SEQ ID NO: 146 - protein, 18 aa, Homo sapiens
MFRLNSLSALAELAVGSR

SEQ ID NO: 147 - protein, 16 aa, Homo sapiens
KADSDSEDKGEESKPK

SEQ ID NO: 148 - protein, 11 aa, Homo sapiens
SASPEHPQKPR

SEQ ID NO: 149 - protein, 19 aa, Homo sapiens
LEQSQKMVIEKEQSLQESK

SEQ ID NO: 150 - protein, 17 aa, Homo sapiens
LGGLRPESPESLTSVSR

SEQ ID NO: 151 - protein, 11 aa, Homo sapiens
SASADNLTLPR

SEQ ID NO: 152 - protein, 21 aa, Homo sapiens
KPSGLNGEASKSQEMVHLVNK

SEQ ID NO: 153 - protein, 10 aa, Homo sapiens
VSNGSPSLER

SEQ ID NO: 154 - protein, 15 aa, Homo sapiens
KTLASKTISISEEVR

SEQ ID NO: 155 - protein, 23 aa, Homo sapiens
SFQSEAPLKRGIVSAQDASLQER

SEQ ID NO: 156 - protein, 15 aa, Homo sapiens
IPEESSDKSPETVNR

SEQ ID NO: 157 - protein, 28 aa, Homo sapiens
YANLSSPTSTVSESQLTKPGVIRPVPVK

SEQ ID NO: 158 - protein, 30 aa, Homo sapiens
VDEEDSDEESHHDEMSEQEEELEDDPTVVK

SEQ ID NO: 159 - protein, 37 aa, Homo sapiens
NMAAMFPMLLSGMAGLPNLLGMGGLLTKPTESGTEDK

SEQ ID NO: 160 - protein, 19 aa, Homo sapiens
ERSPSPLRGNVVPSPLPTR

SEQ ID NO: 161 - protein, 19 aa, Homo sapiens
ERSPSPLRGNVVPSPLPTR

SEQ ID NO: 162 - protein, 11 aa, Homo sapiens
GNVVPSPLPTR

SEQ ID NO: 163 - protein, 7 aa, Homo sapiens
TFSATVR

SEQ ID NO: 164 - protein, 16 aa, Homo sapiens
VGDTEKPEPERSPPNR

SEQ ID NO: 165 - protein, 17 aa, Homo sapiens
GFCPEMGQNESLSEERK

SEQ ID NO: 166 - protein, 13 aa, Homo sapiens
KPESDGRTAKALR

SEQ ID NO: 167 - protein, 24 aa, Homo sapiens
WNFTTPRDEYIEQLKTQMSSCVAK

SEQ ID NO: 168 - protein, 31 aa, Homo sapiens
KESMGPPGLESDPEPAALGSSSAHCTNNGVK

SEQ ID NO: 169 - protein, 31 aa, Homo sapiens; identical to SEQ ID NO: 168

SEQ ID NO: 170 - protein, 26 aa, Homo sapiens
QGLFASVSKISKAVDAPPSSVTSTPR

SEQ ID NO: 171 - protein, 17 aa, Homo sapiens
APDHGGLISKMLPGQDR

SEQ ID NO: 172 - protein, 14 aa, Homo sapiens
KRNSFESQDVPTNK

SEQ ID NO: 173 - protein, 13 aa, Homo sapiens
KSSLGNDETDKEK

SEQ ID NO: 174 - protein, 20 aa, Homo sapiens
RAPAPPPPQPPPPSPLIPNR

SEQ ID NO: 175 - protein, 11 aa, Homo sapiens
VSLGSQIDLQK

SEQ ID NO: 176 - protein, 16 aa, Homo sapiens
GLNGGITPLNSISPLK

SEQ ID NO: 177 - protein, 19 aa, Homo sapiens
AMVSPFHSPPSTPSSPGVR

SEQ ID NO: 178 - protein, 19 aa, Homo sapiens
AMVSPFHSPPSTPSSPGVR

SEQ ID NO: 179 - protein, 19 aa, Homo sapiens
AMVSPFHSPPSTPSSPGVR

SEQ ID NO: 180 - protein, 19 aa, Homo sapiens
AMVSPFHSPPSTPSSPGVR

SEQ ID NO: 181 - protein, 17 aa, Homo sapiens
APGSPLSSEGAAGEGVR

SEQ ID NO: 182 - protein, 30 aa, Homo sapiens
RSSEEVDGQHPAQEEVPESPQTSGPEAENR

SEQ ID NO: 183 - protein, 19 aa, Homo sapiens
SKAPGSPLSSEGAAGEGVR

SEQ ID NO: 184 - protein, 12 aa, Homo sapiens
SQSDCGELGDFR

SEQ ID NO: 185 - protein, 20 aa, Homo sapiens
VDLGQNGEEKSPPNASHPPK

SEQ ID NO: 186 - protein, 9 aa, Homo sapiens
VKSSPLIEK

SEQ ID NO: 187 - protein, 20 aa, Homo sapiens
RDSSESQLASTESDKPTTGR

SEQ ID NO: 188 - protein, 20 aa, Homo sapiens
RDSSESQLASTESDKPTTGR

SEQ ID NO: 189 - protein, 16 aa, Homo sapiens
ASSVTTFTGEPNTCPR

SEQ ID NO: 190 - protein, 11 aa, Homo sapiens
RPFFPFHSPSR

SEQ ID NO: 191 - protein, 9 aa, Homo sapiens
DLPSPLPTK

SEQ ID NO: 192 - protein, 10 aa, Homo sapiens
DLPSPLPTKR

SEQ ID NO: 193 - protein, 11 aa, Homo sapiens
TDGQVSGEAIK

SEQ ID NO: 194 - protein, 9 aa, Homo sapiens
FGESEKCPR

SEQ ID NO: 195 - protein, 31 aa, Homo sapiens
GIGYGQGAGCLSTDTGEHLGLQFQQSPKPAR

SEQ ID NO: 196 - protein, 31 aa, Homo sapiens; identical to SEQ ID NO: 195

SEQ ID NO: 197 - protein, 17 aa, Homo sapiens
SLESTNVTDKDGELYCK

SEQ ID NO: 198 - protein, 14 aa, Homo sapiens
GADSGEEKEEGINR

SEQ ID NO: 199 - protein, 17 aa, Homo sapiens
GADSGEEKEEGINREDK

SEQ ID NO: 200 - protein, 21 aa, Homo sapiens
KLAAGHELQPLAIVDQRPSSR

SEQ ID NO: 201 - protein, 25 aa, Homo sapiens
KLAAGHELQPLAIVDQRPSSRASSR

SEQ ID NO: 202 - protein, 20 aa, Homo sapiens
LAAGHELQPLAIVDQRPSSR

SEQ ID NO: 203 - protein, 36 aa, Homo sapiens
LAAGHELQPLAIVDQRPSSRASSRASSRPRPDDLEI

SEQ ID NO: 204 - protein, 24 aa, Homo sapiens
LAAGHELQPLAIVDQRPSSRASSR

SEQ ID NO: 205 - protein, 12 aa, Homo sapiens
LVTGDRNNSSCR

SEQ ID NO: 206 - protein, 26 aa, Homo sapiens
MGQAGSTISNSHAQPFDFPDDNQNSK

SEQ ID NO: 207 - protein, 26 aa, Homo sapiens
MGQAGSTISNSHAQPFDFPDDNQNSK

SEQ ID NO: 208 - protein, 26 aa, Homo sapiens
MGQAGSTISNSHAQPFDFPDDNQNSK

SEQ ID NO: 209 - protein, 26 aa, Homo sapiens
MGQAGSTISNSHAQPFDFPDDNQNSK

SEQ ID NO: 210 - protein, 26 aa, Homo sapiens
MGQAGSTISNSHAQPFDFPDDNQNSK

SEQ ID NO: 211 - protein, 15 aa, Homo sapiens
SDPYHATSGALSPAK

SEQ ID NO: 212 - protein, 15 aa, Homo sapiens
KTGQAPGYSYTAANK

SEQ ID NO: 213 - protein, 15 aa, Homo sapiens
KTGQAPGYSYTAANK

SEQ ID NO: 214 - protein, 14 aa, Homo sapiens
TGQAPGYSYTAANK

SEQ ID NO: 215 - protein, 25 aa, Homo sapiens
DKDDQEWESPSPPKPTVFISGVIAR

SEQ ID NO: 216 - protein, 21 aa, Homo sapiens
TFGGAPGFPLGSPLSSPVFPR

SEQ ID NO: 217 - protein, 21 aa, Homo sapiens
TFGGAPGFPLGSPLSSPVFPR

SEQ ID NO: 218 - protein, 21 aa, Homo sapiens
TFGGAPGFPLGSPLSSPVFPR

SEQ ID NO: 219 - protein, 25 aa, Homo sapiens
SSSFSDTLEESSPIAAIFDTENLEK

SEQ ID NO: 220 - protein, 17 aa, Homo sapiens
HAPHCLSEEEGEQDRPR

SEQ ID NO: 221 - protein, 17 aa, Homo sapiens
SLSTSGESLYHVLGLDK

SEQ ID NO: 222 - protein, 21 aa, Homo sapiens
LPPNYSMHSAEKVPLQNPPIK

SEQ ID NO: 223 - protein, 17 aa, Homo sapiens
VVPSFLPVDQGGSLVGR

SEQ ID NO: 224 - protein, 11 aa, Homo sapiens
WEEHRSLLSGR

SEQ ID NO: 225 - protein, 16 aa, Homo sapiens
SASSGAEGDVSSEREP

SEQ ID NO: 226 - protein, 34 aa, Homo sapiens
GLAPSVDFQQSVTVVLFQDENTLVSAGAVDGIIK

SEQ ID NO: 227 - protein, 15 aa, Homo sapiens
KVGSENKEEVVELSK

SEQ ID NO: 228 - protein, 8 aa, Homo sapiens
SYTLVVAK

SEQ ID NO: 229 - protein, 12 aa, Homo sapiens
SVRDLEHWHGRK

SEQ ID NO: 230 - protein, 39 aa, Homo sapiens
YGPADVEDTTGSGATDSKDDDDIDLFGSDDEEESEEAKR

SEQ ID NO: 231 - protein, 13 aa, Homo sapiens
ATAPQTQHVSPMR

SEQ ID NO: 232 - protein, 32 aa, Homo sapiens
KPATPAEDDEDDDIDLFGSDNEEEDKEAAQLR

SEQ ID NO: 233 - protein, 35 aa, Homo sapiens
KPATPAEDDEDDDIDLFGSDNEEEDKEAAQLREER

SEQ ID NO: 234 - protein, 25 aa, Homo sapiens
GIPLATGDTSPEPELLPGAPLPPPK

SEQ ID NO: 235 - protein, 25 aa, Homo sapiens
GIPLATGDTSPEPELLPGAPLPPPK

SEQ ID NO: 236 - protein, 17 aa, Homo sapiens
DSAYQSITHYRPVSASR

SEQ ID NO: 237 - protein, 29 aa, Homo sapiens
ALRPPHGPPRPGALTPTPSLSSQALPSPR

SEQ ID NO: 238 - protein, 29 aa, Homo sapiens
ALRPPHGPPRPGALTPTPSLSSQALPSPR

SEQ ID NO: 239 - protein, 14 aa, Homo sapiens
SNSVEKPVSSILSR

SEQ ID NO: 240 - protein, 22 aa, Homo sapiens
KVEEDLKADEPSSEESDLEIDK

SEQ ID NO: 241 - protein, 15 aa, Homo sapiens
RIDFIPVSPAPSPTR

SEQ ID NO: 242 - protein, 9 aa, Homo sapiens
RNSTTFPSR

SEQ ID NO: 243 - protein, 25 aa, Homo sapiens
SNSAPLIHGLSDTSPVFQAEAPSAR

SEQ ID NO: 244 - protein, 23 aa, Homo sapiens
SSSAPLIHGLSDLSQVFQPYTLR

SEQ ID NO: 245 - protein, 16 aa, Homo sapiens
NYSVGSRPLKPLSPLR

SEQ ID NO: 246 - protein, 23 aa, Homo sapiens
EVPQAASKTHGLLQASRPQAQDK

SEQ ID NO: 247 - protein, 32 aa, Homo sapiens
FYEFSIRLEKALQSLLESLTCPPYTPTQHLER

SEQ ID NO: 248 - protein, 8 aa, Homo sapiens
NASVPNLR

SEQ ID NO: 249 - protein, 25 aa, Homo sapiens
GNPEPPVSGEMDDNSLSPEACYECK

SEQ ID NO: 250 - protein, 31 aa, Homo sapiens
SGLPAPWISLRGDDTGVLLPTPGEAQDADLK

SEQ ID NO: 251 - protein, 12 aa, Homo sapiens
CDSSPDSAEDVR

SEQ ID NO: 252 - protein, 13 aa, Homo sapiens
CDSSPDSAEDVRK

SEQ ID NO: 253 - protein, 20 aa, Homo sapiens
HTFMGVVSLGSPSGEVSHPR

SEQ ID NO: 254 - protein, 20 aa, Homo sapiens
HTFMGVVSLGSPSGEVSHPR

SEQ ID NO: 255 - protein, 20 aa, Homo sapiens
HTFMGVVSLGSPSGEVSHPR

SEQ ID NO: 256 - protein, 46 aa, Homo sapiens
VVGRAAEVPGPEPGQQEQLVFGSGDAVELSCPPPGGGPMGPTVWVK

SEQ ID NO: 257 - protein, 7 aa, Homo sapiens
YISFEER

SEQ ID NO: 258 - protein, 44 aa, Homo sapiens
SYPHAKPPYSYISLITMAIQRAPSKMLTLSEIYQWIMDLFPYYR

SEQ ID NO: 259 - protein, 26 aa, Homo sapiens
KMGAPESGLAEYLFDKHTLGDSDNES

SEQ ID NO: 260 - protein, 41 aa, Homo sapiens
AFVGEVGARSAAPGGGSVAAAAAAMGAALGSMVGLMTYGRR

SEQ ID NO: 261 - protein, 24 aa, Homo sapiens
NPPPQDYESDDDSYEVLDLTEYAR

SEQ ID NO: 262 - protein, 18 aa, Homo sapiens
SSSPAPADIAQTVQEDLR

SEQ ID NO: 263 - protein, 15 aa, Homo sapiens
GALQNIIPASTGAAK

SEQ ID NO: 264 - protein, 17 aa, Homo sapiens
IISNASCTTNCLAPLAK

SEQ ID NO: 265 - protein, 24 aa, Homo sapiens
VIHDNFGIVEGLMTTVHAITATQK

SEQ ID NO: 266 - protein, 24 aa, Homo sapiens
VIHDNFGIVEGLMTTVHAITATQK

SEQ ID NO: 267 - protein, 33 aa, Homo sapiens
NTSPTTQPTASGAGAPVMTGAGESTNPENSELK

SEQ ID NO: 268 - protein, 7 aa, Homo sapiens
KSEQLNK

SEQ ID NO: 269 - protein, 18 aa, Homo sapiens
SPSSMVPSILINKAQGSK

SEQ ID NO: 270 - protein, 23 aa, Homo sapiens
FTSQLLDQGTSQCYDVLSSDVQK

SEQ ID NO: 271 - protein, 15 aa, Homo sapiens
LDDQRASVGSLPGLR

SEQ ID NO: 272 - protein, 16 aa, Homo sapiens
GPKPEPPGSGSPAPPR

SEQ ID NO: 273 - protein, 20 aa, Homo sapiens
RLQTTSSRSYVPSAPAGLGK

SEQ ID NO: 274 - protein, 11 aa, Homo sapiens
GEPNVSYICSR

SEQ ID NO: 275 - protein, 16 aa, Homo sapiens
SETAPAAPAAAPPAEK

SEQ ID NO: 276 - protein, 7 aa, Homo sapiens
STELLIR

SEQ ID NO: 277 - protein, 24 aa, Homo sapiens
KSLPAPVAQRPDSPGGGLQAPGQK

SEQ ID NO: 278 - protein, 23 aa, Homo sapiens
SLPAPVAQRPDSPGGGLQAPGQK

SEQ ID NO: 279 - protein, 18 aa, Homo sapiens
KSLDSDESEDEEDDYQQK

SEQ ID NO: 280 - protein, 17 aa, Homo sapiens
SLDSDESEDEEDDYQQK

SEQ ID NO: 281 - protein, 18 aa, Homo sapiens
SLDSDESEDEEDDYQQKR

SEQ ID NO: 282 - protein, 33 aa, Homo sapiens
MLPHAPGVQMQAIPEDAVHEDSGDEDGEDPDKR

SEQ ID NO: 283 - protein, 33 aa, Homo sapiens; identical to SEQ ID NO: 282

SEQ ID NO: 284 - protein, 10 aa, Homo sapiens
AGDLLEDSPK

SEQ ID NO: 285 - protein, 13 aa, Homo sapiens
AGDLLEDSPKRPK

SEQ ID NO: 286 - protein, 20 aa, Homo sapiens
GNAEGSSDEEGKLVIDEPAK

SEQ ID NO: 287 - protein, 22 aa, Homo sapiens
GNAEGSSDEEGKLVIDEPAKEK

SEQ ID NO: 288 - protein, 20 aa, Homo sapiens
GNAEGSSDEEGKLVIDEPAK

SEQ ID NO: 289 - protein, 21 aa, Homo sapiens
KGNAEGSSDEEGKLVIDEPAK

SEQ ID NO: 290 - protein, 11 aa, Homo sapiens
NSTPSEPGSGR

SEQ ID NO: 291 - protein, 11 aa, Homo sapiens
RAGDLLEDSPK

SEQ ID NO: 292 - protein, 14 aa, Homo sapiens
RAGDLLEDSPKRPK

SEQ ID NO: 293 - protein, 10 aa, Homo sapiens
KRSEGFSMDR

SEQ ID NO: 294 - protein, 22 aa, Homo sapiens
TVKMVIPALIQSDSGDSIEVSR

SEQ ID NO: 295 - protein, 19 aa, Homo sapiens
KLEKEEEEGISQESSEEEQ

SEQ ID NO: 296 - protein, 19 aa, Homo sapiens
KLEKEEEEGISQESSEEEQ

SEQ ID NO: 297 - protein, 15 aa, Homo sapiens
RNSSEASSGDFLDLK

SEQ ID NO: 298 - protein, 23 aa, Homo sapiens
REEDEPEERSGDETPGSEVPGDK

SEQ ID NO: 299 - protein, 18 aa, Homo sapiens
IDASKNEEDEGHSNSSPR

SEQ ID NO: 300 - protein, 18 aa, Homo sapiens
IDASKNEEDEGHSNSSPR

SEQ ID NO: 301 - protein, 7 aa, Homo sapiens
DVYLSPR

SEQ ID NO: 302 - protein, 32 aa, Homo sapiens
AKSPQPPVEEEDEHFDDTVVCLDTYNCDLHFK

SEQ ID NO: 303 - protein, 11 aa, Homo sapiens
TVNSTRETPPK

SEQ ID NO: 304 - protein, 20 aa, Homo sapiens
ESEDKPEIEDVGSDEEEEKK

SEQ ID NO: 305 - protein, 24 aa, Homo sapiens
ESEDKPEIEDVGSDEEEEKKDGDK

SEQ ID NO: 306 - protein, 24 aa, Homo sapiens
ESEDKPEIEDVGSDEEEEKKDGDK

SEQ ID NO: 307 - protein, 14 aa, Homo sapiens
IEDVGSDEEDDSGK

SEQ ID NO: 308 - protein, 16 aa, Homo sapiens
IEDVGSDEEDDSGKDK

SEQ ID NO: 309 - protein, 17 aa, Homo sapiens
IEDVGSDEEDDSGKDKK

SEQ ID NO: 310 - protein, 8 aa, Homo sapiens
GPSWDPFR

SEQ ID NO: 311 - protein, 15 aa, Homo sapiens
GPSWDPFRDWYPHSR

SEQ ID NO: 312 - protein, 10 aa, Homo sapiens
QLSSGVSEIR

SEQ ID NO: 313 - protein, 22 aa, Homo sapiens
AERSFHSSSSSSSSSTSSSASR

SEQ ID NO: 314 - protein, 22 aa, Homo sapiens
AERSFHSSSSSSSSSTSSSASR

SEQ ID NO: 315 - protein, 19 aa, Homo sapiens
SFHSSSSSSSSSTSSSASR

SEQ ID NO: 316 - protein, 19 aa, Homo sapiens
SFHSSSSSSSSSTSSSASR

SEQ ID NO: 317 - protein, 19 aa, Homo sapiens
SFHSSSSSSSSSTSSSASR

SEQ ID NO: 318 - protein, 11 aa, Homo sapiens
DPFRDSPLSSR

SEQ ID NO: 319 - protein, 19 aa, Homo sapiens
FGVPAEGRTPPPFPGEPWK

SEQ ID NO: 320 - protein, 18 aa, Homo sapiens
DLDEEGSEKELHENVLDK

SEQ ID NO: 321 - protein, 23 aa, Homo sapiens
ESSPEKEAEEGCPEKESEEGCPK

SEQ ID NO: 322 - protein, 18 aa, Homo sapiens
VFDDESDEKEDEEYADEK

SEQ ID NO: 323 - protein, 16 aa, Homo sapiens
LRRPETSGISDAGGAR

SEQ ID NO: 324 - protein, 10 aa, Homo sapiens
ENKTSEDPSK

SEQ ID NO: 325 - protein, 37 aa, Homo sapiens
EGITGPPADSSKPIGPDDAIDALSSDFTCGSPTAAGK

SEQ ID NO: 326 - protein, 37 aa, Homo sapiens; identical to SEQ ID NO: 325

SEQ ID NO: 327 - protein, 38 aa, Homo sapiens
KEGITGPPADSSKPIGPDDAIDALSSDFTCGSPTAAGK

SEQ ID NO: 328 - protein, 28 aa, Homo sapiens
FEEESKEPVADEEEEDSDDDVEPITEFR

SEQ ID NO: 329 - protein, 18 aa, Homo sapiens
KQSFDDNDSEELEDKDSK

SEQ ID NO: 330 - protein, 19 aa, Homo sapiens
NKPGPNIESGNEDDDASFK

SEQ ID NO: 331 - protein, 18 aa, Homo sapiens
VEMYSGSDDDDDFNKLPK

SEQ ID NO: 332 - protein, 13 aa, Homo sapiens
WDGSEEDEDNSKK

SEQ ID NO: 333 - protein, 28 aa, Homo sapiens
SLENETLNKEEDCHSPTSKPPKPDQPLK

SEQ ID NO: 334 - protein, 17 aa, Homo sapiens
SQSSDTEQQSPTSGGGK

SEQ ID NO: 335 - protein, 16 aa, Homo sapiens
TGSESSQTGTSTTSSR

SEQ ID NO: 336 - protein, 19 aa, Homo sapiens
YLDTGGDVFVMLGEGGESR

SEQ ID NO: 337 - protein, 13 aa, Homo sapiens
SHIAQWSPHTPPR

SEQ ID NO: 338 - protein, 24 aa, Homo sapiens
SFAVGTLAETIQGLGAASAQFVSR

SEQ ID NO: 339 - protein, 18 aa, Homo sapiens
LLESSLSSSEGEEPVEYK

SEQ ID NO: 340 - protein, 18 aa, Homo sapiens
LLESSLSSSEGEEPVEYK

SEQ ID NO: 341 - protein, 17 aa, Homo sapiens
KSQENLGNPSKNEDNVK

SEQ ID NO: 342 - protein, 16 aa, Homo sapiens
AAVLSDSEDEEKASAK

SEQ ID NO: 343 - protein, 17 aa, Homo sapiens
TIASDSEEEAGKELSDK

SEQ ID NO: 344 - protein, 18 aa, Homo sapiens
FAKSNGLEGSWSGNVTQK

SEQ ID NO: 345 - protein, 20 aa, Homo sapiens
ETPRPEGGSPSPAGTPPQPK

SEQ ID NO: 346 - protein, 20 aa, Homo sapiens
GAGAAGLPQPPRESPQLHER

SEQ ID NO: 347 - protein, 10 aa, Homo sapiens
MASPSGKGAR

SEQ ID NO: 348 - protein, 26 aa, Homo sapiens
IQPQPPDEDGDHSDKEDEQPQVVVLK

SEQ ID NO: 349 - protein, 29 aa, Homo sapiens
SKAECDNVGSVENGKTNSVVTCSGAGNGR

SEQ ID NO: 350 - protein, 21 aa, Homo sapiens
KQDDSPPRPIIGPALPPGFIK

SEQ ID NO: 351 - protein, 15 aa, Homo sapiens
RGAFSSVSMSGGAGR

SEQ ID NO: 352 - protein, 23 aa, Homo sapiens
KVNAEGSVDSVFSQVCTHLDALK

SEQ ID NO: 353 - protein, 22 aa, Homo sapiens
VNAEGSVDSVFSQVCTHLDALK

SEQ ID NO: 354 - protein, 13 aa, Homo sapiens
YGYTHLSTGDLLR

SEQ ID NO: 355 - protein, 18 aa, Homo sapiens
TDSREDEISPPPPNPVVK

SEQ ID NO: 356 - protein, 18 aa, Homo sapiens
TDSREDEISPPPPNPVVK

SEQ ID NO: 357 - protein, 18 aa, Homo sapiens
TDSREDEISPPPPNPVVK

SEQ ID NO: 358 - protein, 21 aa, Homo sapiens
RVSVCAETYNPDEEEEDTDPR

SEQ ID NO: 359 - protein, 23 aa, Homo sapiens
VADAKGDSESEEDEDLEVPVPSR

SEQ ID NO: 360 - protein, 21 aa, Homo sapiens
RASVCAEAYNPDEEEDDAESR

SEQ ID NO: 361 - protein, 19 aa, Homo sapiens
TWTLCGTPEYLAPEIILSK

SEQ ID NO: 362 - protein, 19 aa, Homo sapiens
TWTLCGTPEYLAPEIILSK

SEQ ID NO: 363 - protein, 12 aa, Homo sapiens
GQSIDDMIPAQK

SEQ ID NO: 364 - protein, 21 aa, Homo sapiens
GTGGVDTAAVGSVFDVSNADR

SEQ ID NO: 365 - protein, 22 aa, Homo sapiens
RGTGGVDTAAVGSVFDVSNADR

SEQ ID NO: 366 - protein, 22 aa, Homo sapiens
RGTGGVDTAAVGSVFDVSNADR

SEQ ID NO: 367 - protein, 16 aa, Homo sapiens
LGYILTCPSNLGTGLR

SEQ ID NO: 368 - protein, 16 aa, Homo sapiens
LGYILTCPSNLGTGLR

SEQ ID NO: 369 - protein, 19 aa, Homo sapiens
ETTCSKESNEELTESCETK

SEQ ID NO: 370 - protein, 20 aa, Homo sapiens
ETTCSKESNEELTESCETKK

SEQ ID NO: 371 - protein, 19 aa, Homo sapiens
ETTCSKESNEELTESCETK

SEQ ID NO: 372 - protein, 20 aa, Homo sapiens
ETTCSKESNEELTESCETKK

SEQ ID NO: 373 - protein, 19 aa, Homo sapiens
LHISDFSFLMVLGKGSFGK

SEQ ID NO: 374 - protein, 20 aa, Homo sapiens
FASDDEHDEHDENGATGPVK

SEQ ID NO: 375 - protein, 21 aa, Homo sapiens
FASDDEHDEHDENGATGPVKR

SEQ ID NO: 376 - protein, 26 aa, Homo sapiens
GPPDFSSDEEREPTPVLGSGAAAAGR

SEQ ID NO: 377 - protein, 26 aa, Homo sapiens
GPPDFSSDEEREPTPVLGSGAAAAGR

SEQ ID NO: 378 - protein, 16 aa, Homo sapiens
SSTPLPTISSSAENTR

SEQ ID NO: 379 - protein, 14 aa, Homo sapiens
KRSSSEDAESLAPR

SEQ ID NO: 380 - protein, 17 aa, Homo sapiens
SRPTSEGSDIESTEPQK

SEQ ID NO: 381 - protein, 17 aa, Homo sapiens
SRPTSEGSDIESTEPQK

SEQ ID NO: 382 - protein, 17 aa, Homo sapiens
SRPTSEGSDIESTEPQK

SEQ ID NO: 383 - protein, 23 aa, Homo sapiens
MGPSGGEGMEPERRDSQDGSSYR

SEQ ID NO: 384 - protein, 23 aa, Homo sapiens
MGPSGGEGMEPERRDSQDGSSYR

SEQ ID NO: 385 - protein, 19 aa, Homo sapiens
LPSIVVEPTEGEVESGELR

SEQ ID NO: 386 - protein, 13 aa, Homo sapiens
KLTSDEEGEPSGK

SEQ ID NO: 387 - protein, 15 aa, Homo sapiens
QVQTGHKPSTKEIVR

SEQ ID NO: 388 - protein, 48 aa, Homo sapiens
ATVLGDGTLEIRFAQDQDSGMYVCIASNAAGNDTFTASLTVKGFASDR

SEQ ID NO: 389 - protein, 15 aa, Homo sapiens
ETPHSPGVEDAPIAK

SEQ ID NO: 390 - protein, 17 aa, Homo sapiens
GSSDGRGSDSESDLPHR

SEQ ID NO: 391 - protein, 15 aa, Homo sapiens
SPEPEATLTFPFLDK

SEQ ID NO: 392 - protein, 11 aa, Homo sapiens
LRLSPSPTSQR

SEQ ID NO: 393 - protein, 14 aa, Homo sapiens
SGAQASSTPLSPTR

SEQ ID NO: 394 - protein, 12 aa, Homo sapiens
RGESLDNLDSPR

SEQ ID NO: 395 - protein, 16 aa, Homo sapiens
SASVNKEPVSLPGIMR

SEQ ID NO: 396 - protein, 16 aa, Homo sapiens
SASVNKEPVSLPGIMR

SEQ ID NO: 397 - protein, 14 aa, Homo sapiens
SHSPSASQSGSQLR

SEQ ID NO: 398 - protein, 16 aa, Homo sapiens
GSPKPSPQPSPKPSPK

SEQ ID NO: 399 - protein, 16 aa, Homo sapiens
GSPKPSPQPSPKPSPK

SEQ ID NO: 400 - protein, 9 aa, Homo sapiens
NSLSPATQR

SEQ ID NO: 401 - protein, 12 aa, Homo sapiens
GTPSSSPYVSPR

SEQ ID NO: 402 - protein, 26 aa, Homo sapiens
SRPLSPVATPPPPPPPPPPPPPSSQR

SEQ ID NO: 403 - protein, 26 aa, Homo sapiens
SRPLSPVATPPPPPPPPPPPPPSSQR

SEQ ID NO: 404 - protein, 26 aa, Homo sapiens
SRPLSPVATPPPPPPPPPPPPPSSQR

SEQ ID NO: 405 - protein, 26 aa, Homo sapiens
SRPLSPVATPPPPPPPPPPPPPSSQR

SEQ ID NO: 406 - protein, 23 aa, Homo sapiens
YESIDEDELLASLSAEELKELER

SEQ ID NO: 407 - protein, 19 aa, Homo sapiens
DRGLTEFPADLQKLTSNLR

SEQ ID NO: 408 - protein, 10 aa, Homo sapiens
HSLASTDEKR

SEQ ID NO: 409 - protein, 17 aa, Homo sapiens
TKLGWPAGVTLDMISKR

SEQ ID NO: 410 - protein, 19 aa, Homo sapiens
NMPGLSAATLASLGGTSSR

SEQ ID NO: 411 - protein, 20 aa, Homo sapiens
RGSGDTSSLIDPDTSLSELR

SEQ ID NO: 412 - protein, 18 aa, Homo sapiens
ALSRAMLLTSYLPPPLLR

SEQ ID NO: 413 - protein, 33 aa, Homo sapiens
GAPPPGEPGLSHSGSEQPEQTGLLMGGASGGAR

SEQ ID NO: 414 - protein, 33 aa, Homo sapiens; identical to SEQ ID NO: 413

SEQ ID NO: 415 - protein, 11 aa, Homo sapiens
LSSQISAGEEK

SEQ ID NO: 416 - protein, 18 aa, Homo sapiens
SQEPIPDDQKVSDDDKEK

SEQ ID NO: 417 - protein, 19 aa, Homo sapiens
RSSQPSPTAVPASDSPPTK

SEQ ID NO: 418 - protein, 12 aa, Homo sapiens
KESKEETPEVTK

SEQ ID NO: 419 - protein, 19 aa, Homo sapiens
KLGDVSPTQIDVSQFGSFK

SEQ ID NO: 420 - protein, 14 aa, Homo sapiens
SPSLSPSPPSPLEK

SEQ ID NO: 421 - protein, 14 aa, Homo sapiens
DMESPTKLDVTLAK

SEQ ID NO: 422 - protein, 10 aa, Homo sapiens
SKVGSTENIK

SEQ ID NO: 423 - protein, 17 aa, Homo sapiens
VGSLDNVGHLPAGGAVK

SEQ ID NO: 424 - protein, 15 aa, Homo sapiens
AEDGATPSPSNETPK

SEQ ID NO: 425 - protein, 16 aa, Homo sapiens
AEDGATPSPSNETPKK

SEQ ID NO: 426 - protein, 32 aa, Homo sapiens
EAPAEGEAAEPGSPTAAEGEAASAASSTSSPK

SEQ ID NO: 427 - protein, 18 aa, Homo sapiens
GEPAAAAAPEAGASPVEK

SEQ ID NO: 428 - protein, 7 aa, Homo sapiens
LSGFSFK

SEQ ID NO: 429 - protein, 8 aa, Homo sapiens
LSGFSFKK

SEQ ID NO: 430 - protein, 19 aa, Homo sapiens
SSPAAKPGSTPSRPSSAKR

SEQ ID NO: 431 - protein, 22 aa, Homo sapiens
RDSFDDRGPSLNPVLDYDHGSR

SEQ ID NO: 432 - protein, 14 aa, Homo sapiens
SYSPDGKESPSDKK

SEQ ID NO: 433 - protein, 21 aa, Homo sapiens
AETSEGSGSAPAVPEASASPK

SEQ ID NO: 434 - protein, 41 aa, Homo sapiens
SFHAAAYVPAGRGAMYLLGGLTAGGVTRDFWVLNLTTLQWR

SEQ ID NO: 435 - protein, 9 aa, Homo sapiens
YMATQLLAK

SEQ ID NO: 436 - protein, 21 aa, Homo sapiens
AGGANSNVFSMFEQTQIQEFK

SEQ ID NO: 437 - protein, 21 aa, Homo sapiens
AGGANSNVFSMFEQTQIQEFK

SEQ ID NO: 438 - protein, 22 aa, Homo sapiens
RAGGANSNVFSMFEQTQIQEFK

SEQ ID NO: 439 - protein, 22 aa, Homo sapiens
RAGGANSNVFSMFEQTQIQEFK

SEQ ID NO: 440 - protein, 23 aa, Homo sapiens
ALSSLHGDDQDSEDEVLTIPEVK

SEQ ID NO: 441 - protein, 23 aa, Homo sapiens
ALSSLHGDDQDSEDEVLTIPEVK

SEQ ID NO: 442 - protein, 15 aa, Homo sapiens
LVSFHDDSDEDLLHI

SEQ ID NO: 443 - protein, 15 aa, Homo sapiens
LVSFHDDSDEDLLHI

SEQ ID NO: 444 - protein, 15 aa, Homo sapiens
SLQLSTEGFITLTYK

SEQ ID NO: 445 - protein, 18 aa, Homo sapiens
ATSNVFAMFDQSQIQEFK

SEQ ID NO: 446 - protein, 18 aa, Homo sapiens
ATSNVFAMFDQSQIQEFK

SEQ ID NO: 447 - protein, 18 aa, Homo sapiens
ATSNVFAMFDQSQIQEFK

SEQ ID NO: 448 - protein, 18 aa, Homo sapiens
RPYAPINANAIKAECSIR

SEQ ID NO: 449 - protein, 22 aa, Homo sapiens
ATSSSSPRTATTLPVLTSTATK

SEQ ID NO: 450 - protein, 9 aa, Homo sapiens
SSYTHGLNR

SEQ ID NO: 451 - protein, 8 aa, Homo sapiens
QLVSTLEK

SEQ ID NO: 452 - protein, 8 aa, Homo sapiens
EKRTTEHK

SEQ ID NO: 453 - protein, 13 aa, Homo sapiens
DIMLEELSHLSNR

SEQ ID NO: 454 - protein, 17 aa, Homo sapiens
SPPNPDNIAPGYSGPLK

SEQ ID NO: 455 - protein, 24 aa, Homo sapiens
VDGSNLEGGSQQAPLTPPNTPDPR

SEQ ID NO: 456 - protein, 24 aa, Homo sapiens
VDGSNLEGGSQQAPLTPPNTPDPR

SEQ ID NO: 457 - protein, 24 aa, Homo sapiens
VDGSNLEGGSQQAPLTPPNTPDPR

SEQ ID NO: 458 - protein, 18 aa, Homo sapiens
ISDSHEDTGILDFSSLLK

SEQ ID NO: 459 - protein, 19 aa, Homo sapiens
RISDSHEDTGILDFSSLLK

SEQ ID NO: 460 - protein, 15 aa, Homo sapiens
KTGSYGALAEITASK

SEQ ID NO: 461 - protein, 15 aa, Homo sapiens
KTGSYGALAEITASK

SEQ ID NO: 462 - protein, 16 aa, Homo sapiens
RSTQGVTLTDLQEAEK

SEQ ID NO: 463 - protein, 16 aa, Homo sapiens
RSTQGVTLTDLQEAEK

SEQ ID NO: 464 - protein, 15 aa, Homo sapiens
SYLTPVRDEESESQR

SEQ ID NO: 465 - protein, 18 aa, Homo sapiens
DEDETDGSEEVKETWHER

SEQ ID NO: 466 - protein, 10 aa, Homo sapiens
SLDEEPICHR

SEQ ID NO: 467 - protein, 16 aa, Homo sapiens
AAFSKDESKEPIVEVR

SEQ ID NO: 468 - protein, 22 aa, Homo sapiens
RKPSVPDSASPADDSFVDPGER

SEQ ID NO: 469 - protein, 17 aa, Homo sapiens
VMLGETNPADSKPGTIR

SEQ ID NO: 470 - protein, 25 aa, Homo sapiens
SHTSEGAHLDITPNSGAAGNSAGPK

SEQ ID NO: 471 - protein, 23 aa, Homo sapiens
CSTLLVVGDNSPAVEAVVECNSR

SEQ ID NO: 472 - protein, 19 aa, Homo sapiens
TDPGSIFDLDPLEDNIQSR

SEQ ID NO: 473 - protein, 6 aa, Homo sapiens
IQASFR

SEQ ID NO: 474 - protein, 14 aa, Homo sapiens
EMLASDDEEDVSSK

SEQ ID NO: 475 - protein, 17 aa, Homo sapiens
EMLASDDEEDVSSKVEK

SEQ ID NO: 476 - protein, 11 aa, Homo sapiens
TISQEFLTPGK

SEQ ID NO: 477 - protein, 25 aa, Homo sapiens
DPGSPGPFDYVGAPPAESIPQKTRR

SEQ ID NO: 478 - protein, 13 aa, Homo sapiens
GKSPVPKSPVEEK

SEQ ID NO: 479 - protein, 30 aa, Homo sapiens
STEGGFGGTSSSDAQQSLQSFWPRVMEEIR

SEQ ID NO: 480 - protein, 17 aa, Homo sapiens
LKLHTETLNIVFVTYFR

SEQ ID NO: 481 - protein, 11 aa, Homo sapiens
GKGSPRPQAPK

SEQ ID NO: 482 - protein, 15 aa, Homo sapiens
NTALGVISNWTDELR

SEQ ID NO: 483 - protein, 25 aa, Homo sapiens
EFITGDVEPTDAESEWHSENEEEEK

SEQ ID NO: 484 - protein, 31 aa, Homo sapiens
EFITGDVEPTDAESEWHSENEEEEKLAGDMK

SEQ ID NO: 485 - protein, 13 aa, Homo sapiens
KKSPNELVDDLFK

SEQ ID NO: 486 - protein, 13 aa, Homo sapiens
AQRLSQETEALGR

SEQ ID NO: 487 - protein, 20 aa, Homo sapiens
KVVDYSQFQESDDADEDYGR

SEQ ID NO: 488 - protein, 18 aa, Homo sapiens
TPSPKEEDEEPESPPEKK

SEQ ID NO: 489 - protein, 11 aa, Homo sapiens
RSSPPGHYYQK

SEQ ID NO: 490 - protein, 9 aa, Homo sapiens
YGMGTSVER

SEQ ID NO: 491 - protein, 24 aa, Homo sapiens
IGHHSTSDDSSAYRSVDEVNYWDK

SEQ ID NO: 492 - protein, 14 aa, Homo sapiens
IGHHSTSDDSSAYR

SEQ ID NO: 493 - protein, 24 aa, Homo sapiens
IGHHSTSDDSSAYRSVDEVNYWDK

SEQ ID NO: 494 - protein, 31 aa, Homo sapiens
IGHHSTSDDSSAYRSVDEVNYWDKQDHPISR

SEQ ID NO: 495 - protein, 10 aa, Homo sapiens
SVDEVNYWDK

SEQ ID NO: 496 - protein, 17 aa, Homo sapiens
SVDEVNYWDKQDHPISR

SEQ ID NO: 497 - protein, 14 aa, Homo sapiens
YHGHSMSDPGVSYR

SEQ ID NO: 498 - protein, 14 aa, Homo sapiens
YHGHSMSDPGVSYR

SEQ ID NO: 499 - protein, 14 aa, Homo sapiens
YHGHSMSDPGVSYR

SEQ ID NO: 500 - protein, 14 aa, Homo sapiens
YHGHSMSDPGVSYR

SEQ ID NO: 501 - protein, 14 aa, Homo sapiens
YHGHSMSDPGVSYR

SEQ ID NO: 502 - protein, 37 aa, Homo sapiens
LHTPMYIFLGNLVLMDSCCSSAITPKMLENFFSEDKR

SEQ ID NO: 503 - protein, 23 aa, Homo sapiens
TKFPYTNHRTNPGYLLSPVTAQR

SEQ ID NO: 504 - protein, 45 aa, Homo sapiens
DLNHYMEQILNVSHEVTNSDCVLSFFLSEAVQQTVESSPVYLGEK

SEQ ID NO: 505 - protein, 29 aa, Homo sapiens
DGTAPPPQSPGSPGTGQDEEWSDEESPRK

SEQ ID NO: 506 - protein, 17 aa, Homo sapiens
GGRSPDEVTLTSIVPTR

SEQ ID NO: 507 - protein, 25 aa, Homo sapiens
NSKSPTEYHEPVYANPFYRPTTPQR

SEQ ID NO: 508 - protein, 18 aa, Homo sapiens
SEHQNSSPTCQEDEEDVR

SEQ ID NO: 509 - protein, 22 aa, Homo sapiens
SPTEYHEPVYANPFYRPTTPQR

SEQ ID NO: 510 - protein, 24 aa, Homo sapiens
ENHADLGIFVKSIINGGAASKDGR

SEQ ID NO: 511 - protein, 20 aa, Homo sapiens
SNSPLPSIQLQPQSPSASKK

SEQ ID NO: 512 - protein, 9 aa, Homo sapiens
LSDSPSMGR

SEQ ID NO: 513 - protein, 17 aa, Homo sapiens
NIGRDTPTSAGPNSFNK

SEQ ID NO: 514 - protein, 7 aa, Homo sapiens
SPSPSFR

SEQ ID NO: 515 - protein, 12 aa, Homo sapiens
TSPPCSPANLSR

SEQ ID NO: 516 - protein, 8 aa, Homo sapiens
MLQALSPK

SEQ ID NO: 517 - protein, 10 aa, Homo sapiens
SGLTVPTSPK

SEQ ID NO: 518 - protein, 13 aa, Homo sapiens
RSSLNSISSSDAK

SEQ ID NO: 519 - protein, 9 aa, Homo sapiens
RTSLPCIPR

SEQ ID NO: 520 - protein, 29 aa, Homo sapiens
SSRPSMDSEGGSLLLDEDSEVFKMLQENR

SEQ ID NO: 521 - protein, 15 aa, Homo sapiens
NRPTSISWDGLDSGK

SEQ ID NO: 522 - protein, 15 aa, Homo sapiens
NRPTSISWDGLDSGK

SEQ ID NO: 523 - protein, 23 aa, Homo sapiens
AIGGIILTASHNPGGPNGDFGIK

SEQ ID NO: 524 - protein, 8 aa, Homo sapiens
KAVLPTSK

SEQ ID NO: 525 - protein, 19 aa, Homo sapiens
SALKDTYMLSSTVSSKILR

SEQ ID NO: 526 - protein, 23 aa, Homo sapiens
AAGGIILTASHCPGGPGGEFGVK

SEQ ID NO: 527 - protein, 20 aa, Homo sapiens
EGEEPTVYSDEEEPKDESAR

SEQ ID NO: 528 - protein, 23 aa, Homo sapiens
LLKEGEEPTVYSDEEEPKDESAR

SEQ ID NO: 529 - protein, 24 aa, Homo sapiens
LLKPGEEPSEYTDEEDTKDHNKQD

SEQ ID NO: 530 - protein, 18 aa, Homo sapiens
LLKPGEEPSEYTDEEDTK

SEQ ID NO: 531 - protein, 22 aa, Homo sapiens
LLKPGEEPSEYTDEEDTKDHNK

SEQ ID NO: 532 - protein, 24 aa, Homo sapiens
LLKPGEEPSEYTDEEDTKDHNKQD

SEQ ID NO: 533 - protein, 15 aa, Homo sapiens
DRSPHRSSPSDTRPK

SEQ ID NO: 534 - protein, 16 aa, Homo sapiens
SLSPGKENVSALDMEK

SEQ ID NO: 535 - protein, 12 aa, Homo sapiens
RLEISPDSSPER

SEQ ID NO: 536 - protein, 13 aa, Homo sapiens
KTVSFSSMPSEKK

SEQ ID NO: 537 - protein, 16 aa, Homo sapiens
DPYSGSTISLFQAMQK

SEQ ID NO: 538 - protein, 16 aa, Homo sapiens
TGEPDEEEGTFRSSIR

SEQ ID NO: 539 - protein, 36 aa, Homo sapiens
NPCEDSFLPDTEGHTYVAFIRMEKDDDFTTWTQLAK

SEQ ID NO: 540 - protein, 6 aa, Homo sapiens
RLSSLR

SEQ ID NO: 541 - protein, 16 aa, Homo sapiens
GTSSMSSLHVSSPHQR

SEQ ID NO: 542 - protein, 16 aa, Homo sapiens
GTSSMSSLHVSSPHQR

SEQ ID NO: 543 - protein, 16 aa, Homo sapiens
GTSSMSSLHVSSPHQR

SEQ ID NO: 544 - protein, 24 aa, Homo sapiens
MKPIEEGAEDDDDVFEPASPNTLK

SEQ ID NO: 545 - protein, 24 aa, Homo sapiens
MKPIEEGAEDDDDVFEPASPNTLK

SEQ ID NO: 546 - protein, 18 aa, Homo sapiens
NSIASSSDSDDGLHQFLR

SEQ ID NO: 547 - protein, 18 aa, Homo sapiens
NSIASSSDSDDGLHQFLR

SEQ ID NO: 548 - protein, 18 aa, Homo sapiens
NSIASSSDSDDGLHQFLR

SEQ ID NO: 549 - protein, 13 aa, Homo sapiens
GQAPLAPTHTPEL

SEQ ID NO: 550 - protein, 12 aa, Homo sapiens
IPLQSYSQVISR

SEQ ID NO: 551 - protein, 12 aa, Homo sapiens
LSRPDSGILASR

SEQ ID NO: 552 - protein, 12 aa, Homo sapiens
MSTQERLKHAQK

SEQ ID NO: 553 - protein, 14 aa, Homo sapiens
DTSPSSGSAVSSSK

SEQ ID NO: 554 - protein, 12 aa, Homo sapiens
RASTIEMPQQAR

SEQ ID NO: 555 - protein, 12 aa, Homo sapiens
RASTIEMPQQAR

SEQ ID NO: 556 - protein, 15 aa, Homo sapiens
RGPNTQGELQNASPK

SEQ ID NO: 557 - protein, 13 aa, Homo sapiens
RRSQSIEQESQEK

SEQ ID NO: 558 - protein, 10 aa, Homo sapiens
EDSGTFSLGK

SEQ ID NO: 559 - protein, 20 aa, Homo sapiens
HRAEAPPLEREDSGTFSLGK

SEQ ID NO: 560 - protein, 10 aa, Homo sapiens
KKSPIINESR

SEQ ID NO: 561 - protein, 9 aa, Homo sapiens
RRSLSPKPR

SEQ ID NO: 562 - protein, 9 aa, Homo sapiens
SRSPLLNDR

SEQ ID NO: 563 - protein, 10 aa, Homo sapiens
SRSPLLNDRR

SEQ ID NO: 564 - protein, 14 aa, Homo sapiens
ESLKEEDESDDDNM

SEQ ID NO: 565 - protein, 14 aa, Homo sapiens
ESLKEEDESDDDNM

SEQ ID NO: 566 - protein, 14 aa, Homo sapiens
AISTGSLASSTLNK

SEQ ID NO: 567 - protein, 25 aa, Homo sapiens
VGLAPLPGMKGTDYINASYIMGYYR

SEQ ID NO: 568 - protein, 44 aa, Homo sapiens
ESEALPEKEGEELGEGERPEEDAAALELSSDEAVEVEEVIEESR

SEQ ID NO: 569 - protein, 44 aa, Homo sapiens; identical to SEQ ID NO: 568

SEQ ID NO: 570 - protein, 7 aa, Homo sapiens
KVSVNVK

SEQ ID NO: 571 - protein, 9 aa, Homo sapiens
LPAKLSISK

SEQ ID NO: 572 - protein, 24 aa, Homo sapiens
RGSSPDVHALLEITEESDAVLVDK

SEQ ID NO: 573 - protein, 11 aa, Homo sapiens
SFTPDHVVYAR

SEQ ID NO: 574 - protein, 11 aa, Homo sapiens
SLKESEALPEK

SEQ ID NO: 575 - protein, 18 aa, Homo sapiens
VMIYQDEVKLPAKLSISK

SEQ ID NO: 576 - protein, 9 aa, Homo sapiens
EIPTPEPLK

SEQ ID NO: 577 - protein, 22 aa, Homo sapiens
LSSVTEDEDQDAALTIVTVLDK

SEQ ID NO: 578 - protein, 33 aa, Homo sapiens
SESLGPISELYSDELSEPEHEAARPVYPPHEGR

SEQ ID NO: 579 - protein, 10 aa, Homo sapiens
SGKEHIDNIK

SEQ ID NO: 580 - protein, 11 aa, Homo sapiens
SGKEHIDNIKK

SEQ ID NO: 581 - protein, 33 aa, Homo sapiens; identical to SEQ ID NO: 578

SEQ ID NO: 582 - protein, 33 aa, Homo sapiens; identical to SEQ ID NO: 578

SEQ ID NO: 583 - protein, 17 aa, Homo sapiens
RGSGHPAYAEVEPVGEK

SEQ ID NO: 584 - protein, 18 aa, Homo sapiens
SHTEEDCTEELFDFLHAR

SEQ ID NO: 585 - protein, 18 aa, Homo sapiens
SHTEEDCTEELFDFLHAR

SEQ ID NO: 586 - protein, 33 aa, Homo sapiens
QHYGRDNLLDTYSADQGDSSEGGTLARGEEEEK

SEQ ID NO: 587 - protein, 11 aa, Homo sapiens
SESPKEPEQLR

SEQ ID NO: 588 - protein, 12 aa, Homo sapiens
SESPKEPEQLRK

SEQ ID NO: 589 - protein, 14 aa, Homo sapiens
RGSTPWGPAPPLHR

SEQ ID NO: 590 - protein, 15 aa, Homo sapiens
RRGSTPWGPAPPLHR

SEQ ID NO: 591 - protein, 10 aa, Homo sapiens
SKSCHDLSVL

SEQ ID NO: 592 - protein, 10 aa, Homo sapiens
GRLSPVPVPR

SEQ ID NO: 593 - protein, 12 aa, Homo sapiens
LEVTEIVKPSPK

SEQ ID NO: 594 - protein, 11 aa, Homo sapiens
LASDDRPSPPR

SEQ ID NO: 595 - protein, 14 aa, Homo sapiens
LGASNSPGQPNSVK

SEQ ID NO: 596 - protein, 17 aa, Homo sapiens
DKSPVREPIDNLTPEER

SEQ ID NO: 597 - protein, 12 aa, Homo sapiens
AGKEPAKPSPSR

SEQ ID NO: 598 - protein, 20 aa, Homo sapiens
KLSGDQITLPTTVDYSSVPK

SEQ ID NO: 599 - protein, 23 aa, Homo sapiens
FQVDMTLDADTANNFLLISDDLR

SEQ ID NO: 600 - protein, 32 aa, Homo sapiens
SLDLDHSMDEAGAGASNSEPSEPDSPTREHAR

SEQ ID NO: 601 - protein, 24 aa, Homo sapiens
SHLAEAFAKAKGSGGGAGGGGSGR

SEQ ID NO: 602 - protein, 17 aa, Homo sapiens
ATNESEDEIPQLVPIGK

SEQ ID NO: 603 - protein, 17 aa, Homo sapiens
ATNESEDEIPQLVPIGK

SEQ ID NO: 604 - protein, 17 aa, Homo sapiens
KEESEESDDDMGFGLFD

SEQ ID NO: 605 - protein, 17 aa, Homo sapiens
KEESEESDDDMGFGLFD

SEQ ID NO: 606 - protein, 17 aa, Homo sapiens
KNSTGSGHSAQELPTIR

SEQ ID NO: 607 - protein, 6 aa, Homo sapiens
RFSPPR

SEQ ID NO: 608 - protein, 22 aa, Homo sapiens
SSGSPYGGGYGSGGGSGGYGSR

SEQ ID NO: 609 - protein, 17 aa, Homo sapiens
SVIGSVTLVSETEVQEK

SEQ ID NO: 610 - protein, 15 aa, Homo sapiens
KFQEQECPPSPEPTR

SEQ ID NO: 611 - protein, 16 aa, Homo sapiens
KFQEQECPPSPEPTRK

SEQ ID NO: 612 - protein, 10 aa, Homo sapiens
EQSEVSVSPR

SEQ ID NO: 613 - protein, 11 aa, Homo sapiens
LSSSLGRSSGR

SEQ ID NO: 614 - protein, 12 aa, Homo sapiens
DTDISIKPEGVR

SEQ ID NO: 615 - protein, 13 aa, Homo sapiens
YDERPGPSPLPHR

SEQ ID NO: 616 - protein, 19 aa, Homo sapiens
MHTAVKLNEVIVTRSHDAR

SEQ ID NO: 617 - protein, 10 aa, Homo sapiens
RSLTNSHLEK

SEQ ID NO: 618 - protein, 7 aa, Homo sapiens
SVVSFDK

SEQ ID NO: 619 - protein, 9 aa, Homo sapiens
SVVSFDKVK

SEQ ID NO: 620 - protein, 19 aa, Homo sapiens
PGPTPSGTNVGSSGRSPSK

SEQ ID NO: 621 - protein, 19 aa, Homo sapiens
PGPTPSGTNVGSSGRSPSK

SEQ ID NO: 622 - protein, 44 aa, Homo sapiens
EELPDENKSLEETLHTVDLSSDDDLPHDEEALEDSAEEKVEESR

SEQ ID NO: 623 - protein, 18 aa, Homo sapiens
ISSGKSSPFKVSPLTFGR

SEQ ID NO: 624 - protein, 18 aa, Homo sapiens
ISSGKSSPFKVSPLTFGR

SEQ ID NO: 625 - protein, 13 aa, Homo sapiens
SSPFKVSPLTFGR

SEQ ID NO: 626 - protein, 36 aa, Homo sapiens
SLEETLHTVDLSSDDDLPHDEEALEDSAEEKVEESR

SEQ ID NO: 627 - protein, 31 aa, Homo sapiens
SLEETLHTVDLSSDDDLPHDEEALEDSAEEK

SEQ ID NO: 628 - protein, 36 aa, Homo sapiens; identical to SEQ ID NO: 626

SEQ ID NO: 629 - protein, 36 aa, Homo sapiens; identical to SEQ ID NO: 626

SEQ ID NO: 630 - protein, 13 aa, Homo sapiens
SSPFKVSPLTFGR

SEQ ID NO: 631 - protein, 8 aa, Homo sapiens
VSPLTFGR

SEQ ID NO: 632 - protein, 23 aa, Homo sapiens
VGPGNHGTEGSGGERHSDTDSDR

SEQ ID NO: 633 - protein, 20 aa, Homo sapiens
IYHLPDAESDEDEDFKEQTR

SEQ ID NO: 634 - protein, 11 aa, Homo sapiens
VDGPRSPSYGR

SEQ ID NO: 635 - protein, 13 aa, Homo sapiens
VDGPRSPSYGRSR

SEQ ID NO: 636 - protein, 11 aa, Homo sapiens
VDGPRSPSYGR

SEQ ID NO: 637 - protein, 13 aa, Homo sapiens
VKVDGPRSPSYGR

SEQ ID NO: 638 - protein, 13 aa, Homo sapiens
VKVDGPRSPSYGR

SEQ ID NO: 639 - protein, 9 aa, Homo sapiens
ARSVSPPPK

SEQ ID NO: 640 - protein, 10 aa, Homo sapiens
ARSVSPPPKR

SEQ ID NO: 641 - protein, 12 aa, Homo sapiens
GSPHYFSPFRPY

SEQ ID NO: 642 - protein, 17 aa, Homo sapiens
LPPRVDSAQVPLILDQH

SEQ ID NO: 643 - protein, 10 aa, Homo sapiens
FQNYSPPPTK

SEQ ID NO: 644 - protein, 15 aa, Homo sapiens
KDFPSNSFYVVVVVK

SEQ ID NO: 645 - protein, 18 aa, Homo sapiens
AGAGMITQHSSNASPINR

SEQ ID NO: 646 - protein, 18 aa, Homo sapiens
AGAGMITQHSSNASPINR

SEQ ID NO: 647 - protein, 12 aa, Homo sapiens
DGQDAIAQSPEK

SEQ ID NO: 648 - protein, 15 aa, Homo sapiens
DGQDAIAQSPEKESK

SEQ ID NO: 649 - protein, 12 aa, Homo sapiens
SPGHMVILDQTK

SEQ ID NO: 650 - protein, 23 aa, Homo sapiens
SASPDDDLGSSNWEAADLGNEER

SEQ ID NO: 651 - protein, 24 aa, Homo sapiens
SASPDDDLGSSNWEAADLGNEERK

SEQ ID NO: 652 - protein, 14 aa, Homo sapiens
GRPPAEKLSPNPPK

SEQ ID NO: 653 - protein, 13 aa, Homo sapiens
STSFGVPNANSIK

SEQ ID NO: 654 - protein, 26 aa, Homo sapiens
VVATSLPSGGGCSTCGGCSPRMKVTK

SEQ ID NO: 655 - protein, 8 aa, Homo sapiens
LASSRVER

SEQ ID NO: 656 - protein, 39 aa, Homo sapiens
VGGSGGGGHGGGGGGGSSNAGGGGGGASGGGANSKPAQK

SEQ ID NO: 657 - protein, 24 aa, Homo sapiens
LNECISPVANEMNHLPAHSHDLQR

SEQ ID NO: 658 - protein, 24 aa, Homo sapiens
LNECISPVANEMNHLPAHSHDLQR

SEQ ID NO: 659 - protein, 14 aa, Homo sapiens
LPLQESEEEEREER

SEQ ID NO: 660 - protein, 14 aa, Homo sapiens
ELKELASRVAFLTK

SEQ ID NO: 661 - protein, 21 aa, Homo sapiens
AEDEENEKETAVSTEDDSHHK

SEQ ID NO: 662 - protein, 34 aa, Homo sapiens
DQGNQEQDPNISNGEEEEEKEPGEVGTHNDNQER

SEQ ID NO: 663 - protein, 13 aa, Homo sapiens
HIQETEWQSQEGK

SEQ ID NO: 664 - protein, 9 aa, Homo sapiens
SSSQELGLK

SEQ ID NO: 665 - protein, 8 aa, Homo sapiens
KELIDYLR

SEQ ID NO: 666 - protein, 21 aa, Homo sapiens
DASSPVPPPHVPPPVPPLRPR

SEQ ID NO: 667 - protein, 21 aa, Homo sapiens
DASSPVPPPHVPPPVPPLRPR

SEQ ID NO: 668 - protein, 18 aa, Homo sapiens
GAEDYPDPPIPHSYSSDR

SEQ ID NO: 669 - protein, 11 aa, Homo sapiens
RKSEPAVGPPR

SEQ ID NO: 670 - protein, 9 aa, Homo sapiens
SEPAVGPPR

SEQ ID NO: 671 - protein, 12 aa, Homo sapiens
SFTSSSPSSPSR

SEQ ID NO: 672 - protein, 12 aa, Homo sapiens
SFTSSSPSSPSR

SEQ ID NO: 673 - protein, 11 aa, Homo sapiens
SHSDNSPNAFK

SEQ ID NO: 674 - protein, 17 aa, Homo sapiens
TSPGRVDLPGSSTTLTK

SEQ ID NO: 675 - protein, 17 aa, Homo sapiens
TSPGRVDLPGSSTTLTK

SEQ ID NO: 676 - protein, 18 aa, Homo sapiens
AKTQTPPVSPAPQPTEER

SEQ ID NO: 677 - protein, 18 aa, Homo sapiens
AKTQTPPVSPAPQPTEER

SEQ ID NO: 678 - protein, 32 aa, Homo sapiens
ASAGHAVSIAQDDAGADDWETDPDFVNDVSEK

SEQ ID NO: 679 - protein, 9 aa, Homo sapiens
HCSQVDSVR

SEQ ID NO: 680 - protein, 14 aa, Homo sapiens
LPSSPVYEDAASFK

SEQ ID NO: 681 - protein, 14 aa, Homo sapiens
LPSSPVYEDAASFK

SEQ ID NO: 682 - protein, 16 aa, Homo sapiens
TQTPPVSPAPQPTEER

SEQ ID NO: 683 - protein, 16 aa, Homo sapiens
TQTPPVSPAPQPTEER

SEQ ID NO: 684 - protein, 16 aa, Homo sapiens
TQTPPVSPAPQPTEER

SEQ ID NO: 685 - protein, 28 aa, Homo sapiens
AEVGAPLSPDHSEEEEEEEEGLEEDEPR

SEQ ID NO: 686 - protein, 28 aa, Homo sapiens
AEVGAPLSPDHSEEEEEEEEGLEEDEPR

SEQ ID NO: 687 - protein, 28 aa, Homo sapiens
AEVGAPLSPDHSEEEEEEEEGLEEDEPR

SEQ ID NO: 688 - protein, 28 aa, Homo sapiens
DDSEEEKEKEEDPGSHEEDDESSEQGEK

SEQ ID NO: 689 - protein, 12 aa, Homo sapiens
DEEEDEDVSTER

SEQ ID NO: 690 - protein, 41 aa, Homo sapiens
EEAGGASSEEESGEDTGPQDAQEYGNYQPGSLCGYCSFCNR

SEQ ID NO: 691 - protein, 18 aa, Homo sapiens
EEDEEVSAELGHQAPSHR

SEQ ID NO: 692 - protein, 21 aa, Homo sapiens
EKEEDPGSHEEDDESSEQGEK

SEQ ID NO: 693 - protein, 32 aa, Homo sapiens; identical to SEQ ID NO: 29

SEQ ID NO: 694 - protein, 22 aa, Homo sapiens
GHGSEEDEDVSDGHHHHGPSHR

SEQ ID NO: 695 - protein, 14 aa, Homo sapiens
GHGSEDTEDSAEHR

SEQ ID NO: 696 - protein, 14 aa, Homo sapiens
GHGSEDTEDSAEHR

SEQ ID NO: 697 - protein, 14 aa, Homo sapiens
GHKSDEEDFQDEYK

SEQ ID NO: 698 - protein, 28 aa, Homo sapiens
HQGHEEDDDDDDDDDDDDDDDDVSIEYR

SEQ ID NO: 699 - protein, 17 aa, Homo sapiens
HQGHRDEEEDEDVSTER

SEQ ID NO: 700 - protein, 24 aa, Homo sapiens
HRSHEEDDNDDDDVSTEYGHQAHR

SEQ ID NO: 701 - protein, 24 aa, Homo sapiens
HRSHEEDDNDDDDVSTEYGHQAHR

SEQ ID NO: 702 - protein, 22 aa, Homo sapiens
SHEEDDNDDDDVSTEYGHQAHR

SEQ ID NO: 703 - protein, 20 aa, Homo sapiens
VGDEGVSGEEVFAEHGGQAR

SEQ ID NO: 704 - protein, 21 aa, Homo sapiens
VPREEDEEVSAELGHQAPSHR

SEQ ID NO: 705 - protein, 10 aa, Homo sapiens
AASPSPQSVR

SEQ ID NO: 706 - protein, 10 aa, Homo sapiens
AASPSPQSVR

SEQ ID NO: 707 - protein, 12 aa, Homo sapiens
APQTSSSPPPVR

SEQ ID NO: 708 - protein, 12 aa, Homo sapiens
HRPSPPATPPPK

SEQ ID NO: 709 - protein, 13 aa, Homo sapiens
KEKTPELPEPSVK

SEQ ID NO: 710 - protein, 16 aa, Homo sapiens
KETESEAEDNLDDLEK

SEQ ID NO: 711 - protein, 16 aa, Homo sapiens
KETESEAEDNLDDLEK

SEQ ID NO: 712 - protein, 25 aa, Homo sapiens
KPPAPPSPVQSQSPSTNWSPAVPVK

SEQ ID NO: 713 - protein, 26 aa, Homo sapiens
KPPAPPSPVQSQSPSTNWSPAVPVKK

SEQ ID NO: 714 - protein, 10 aa, Homo sapiens
KSRVSVSPGR

SEQ ID NO: 715 - protein, 14 aa, Homo sapiens
KVELSESEEDKGGK

SEQ ID NO: 716 - protein, 14 aa, Homo sapiens
KVELSESEEDKGGK

SEQ ID NO: 717 - protein, 11 aa, Homo sapiens
RESPSPAPKPR

SEQ ID NO: 718 - protein, 11 aa, Homo sapiens
RESPSPAPKPR

SEQ ID NO: 719 - protein, 11 aa, Homo sapiens
RESPSPAPKPR

SEQ ID NO: 720 - protein, 10 aa, Homo sapiens
RLSPSASPPR

SEQ ID NO: 721 - protein, 12 aa, Homo sapiens
RQSPSPSTRPIR

SEQ ID NO: 722 - protein, 10 aa, Homo sapiens
RRASPSPPPK

SEQ ID NO: 723 - protein, 11 aa, Homo sapiens
RRSPSPAPPPR

SEQ ID NO: 724 - protein, 10 aa, Homo sapiens
RRSPSPPPTR

SEQ ID NO: 725 - protein, 11 aa, Homo sapiens
RRTASPPPPPK

SEQ ID NO: 726 - protein, 9 aa, Homo sapiens
RRTPSPPPR

SEQ ID NO: 727 - protein, 9 aa, Homo sapiens
RRTPTPPPR

SEQ ID NO: 728 - protein, 10 aa, Homo sapiens
RYSPSPPPKR

SEQ ID NO: 729 - protein, 9 aa, Homo sapiens
SPSPAPPPR

SEQ ID NO: 730 - protein, 9 aa, Homo sapiens
SRVSVSPGR

SEQ ID NO: 731 - protein, 9 aa, Homo sapiens
TASPPPPPK

SEQ ID NO: 732 - protein, 17 aa, Homo sapiens
VPKPEPIPEPKEPSPEK

SEQ ID NO: 733 - protein, 13 aa, Homo sapiens
AQTPPGPSLSGSK

SEQ ID NO: 734 - protein, 20 aa, Homo sapiens
AQTPPGPSLSGSKSPCPQEK

SEQ ID NO: 735 - protein, 20 aa, Homo sapiens
AQTPPGPSLSGSKSPCPQEK

SEQ ID NO: 736 - protein, 10 aa, Homo sapiens
GEFSASPMLK

SEQ ID NO: 737 - protein, 26 aa, Homo sapiens
GEGDAPFSEPGTTSTQRPSSPETATK

SEQ ID NO: 738 - protein, 19 aa, Homo sapiens
HASSSPESPKPAPAPGSHR

SEQ ID NO: 739 - protein, 28 aa, Homo sapiens
HGGSPQPLATTPLSQEPVNPPSEASPTR

SEQ ID NO: 740 - protein, 16 aa, Homo sapiens
NHSGSRTPPVALNSSR

SEQ ID NO: 741 - protein, 16 aa, Homo sapiens
NHSGSRTPPVALNSSR

SEQ ID NO: 742 - protein, 27 aa, Homo sapiens
RGEGDAPFSEPGTTSTQRPSSPETATK

SEQ ID NO: 743 - protein, 27 aa, Homo sapiens
RGEGDAPFSEPGTTSTQRPSSPETATK

SEQ ID NO: 744 - protein, 9 aa, Homo sapiens
RPSPQPSPR

SEQ ID NO: 745 - protein, 10 aa, Homo sapiens
RRPSPQPSPR

SEQ ID NO: 746 - protein, 10 aa, Homo sapiens
RRPSPQPSPR

SEQ ID NO: 747 - protein, 10 aa, Homo sapiens
RVPSPTPAPK

SEQ ID NO: 748 - protein, 12 aa, Homo sapiens
SRSPSSPELNNK

SEQ ID NO: 749 - protein, 11 aa, Homo sapiens
SATRPSPSPER

SEQ ID NO: 750 - protein, 12 aa, Homo sapiens
SCFESSPDPELK

SEQ ID NO: 751 - protein, 12 aa, Homo sapiens
SRTPPSAPSQSR

SEQ ID NO: 752 - protein, 11 aa, Homo sapiens
SSSPVTELASR

SEQ ID NO: 753 - protein, 18 aa, Homo sapiens
SSTPPGESYFGVSSLQLK

SEQ ID NO: 754 - protein, 18 aa, Homo sapiens
SSTPPGESYFGVSSLQLK

SEQ ID NO: 755 - protein, 17 aa, Homo sapiens
THTTALAGRSPSPASGR

SEQ ID NO: 756 - protein, 17 aa, Homo sapiens
THTTALAGRSPSPASGR

SEQ ID NO: 757 - protein, 18 aa, Homo sapiens
THTTALAGRSPSPASGRR

SEQ ID NO: 758 - protein, 12 aa, Homo sapiens
VSGRTSPPLLDR

SEQ ID NO: 759 - protein, 25 aa, Homo sapiens
HKGSGRTGGLVISGPMLQQEPESFK

SEQ ID NO: 760 - protein, 14 aa, Homo sapiens
RASGQAFELILSPR

SEQ ID NO: 761 - protein, 18 aa, Homo sapiens
LGAGGGSPEKSPSAQELK

SEQ ID NO: 762 - protein, 24 aa, Homo sapiens
QSMSRIPASSNSTSSKLTHINNSR

SEQ ID NO: 763 - protein, 17 aa, Homo sapiens
SVSTQKALEEDLQIATK

SEQ ID NO: 764 - protein, 30 aa, Homo sapiens
TFFSFPAVVAPFKCSVLPLSQNQEFMPFVK

SEQ ID NO: 765 - protein, 13 aa, Homo sapiens
AQSPTPSLPASWK

SEQ ID NO: 766 - protein, 13 aa, Homo sapiens
AQSPTPSLPASWK

SEQ ID NO: 767 - protein, 18 aa, Homo sapiens
AASPAKPSSLDLVPNLPK

SEQ ID NO: 768 - protein, 8 aa, Homo sapiens
VYLLPDKR

SEQ ID NO: 769 - protein, 11 aa, Homo sapiens
SPVVSGDTSPR

SEQ ID NO: 770 - protein, 21 aa, Homo sapiens
TDHGAEIVYKSPVVSGDTSPR

SEQ ID NO: 771 - protein, 16 aa, Homo sapiens
GTDDSPKDSQEDLQER

SEQ ID NO: 772 - protein, 34 aa, Homo sapiens
NEGNLENEGKPEDEVEPDDEGKSDEEEKPDVEGK

SEQ ID NO: 773 - protein, 15 aa, Homo sapiens
KKEPAITSQNSPEAR

SEQ ID NO: 774 - protein, 17 aa, Homo sapiens
GLECSDWKPEAGLSPPR

SEQ ID NO: 775 - protein, 10 aa, Homo sapiens
KLSGDQPAAR

SEQ ID NO: 776 - protein, 16 aa, Homo sapiens
SLGNILQAKPTSSPAK

SEQ ID NO: 777 - protein, 16 aa, Homo sapiens
TSQVGAASAPAKESPR

SEQ ID NO: 778 - protein, 15 aa, Homo sapiens
DWEDDSDEDMSNFDR

SEQ ID NO: 779 - protein, 27 aa, Homo sapiens
LNWLSVDFNNWKDWEDDSDEDMSNFDR

SEQ ID NO: 780 - protein, 27 aa, Homo sapiens
LNWLSVDFNNWKDWEDDSDEDMSNFDR

SEQ ID NO: 781 - protein, 27 aa, Homo sapiens
EEREDTPIQLQELLALETALGGQCVDR

SEQ ID NO: 782 - protein, 9 aa, Homo sapiens
SMSQEAQRG

SEQ ID NO: 783 - protein, 18 aa, Homo sapiens
AQFSVAGVHTVPGSPQAR

SEQ ID NO: 784 - protein, 12 aa, Homo sapiens
VATTPGSPSLGR

SEQ ID NO: 785 - protein, 19 aa, Homo sapiens
DSPSKSSAEAQTPEDTPNK

SEQ ID NO: 786 - protein, 19 aa, Homo sapiens
DSPSKSSAEAQTPEDTPNK

SEQ ID NO: 787 - protein, 24 aa, Homo sapiens
FTDKDQQPSGSEGEDDDAEAALKK

SEQ ID NO: 788 - protein, 10 aa, Homo sapiens
QDSKSTSPGK

SEQ ID NO: 789 - protein, 14 aa, Homo sapiens
AVSPTETKPTPTEK

SEQ ID NO: 790 - protein, 7 aa, Homo sapiens
AVTSPPR

SEQ ID NO: 791 - protein, 11 aa, Homo sapiens
IELSPSMEAPK

SEQ ID NO: 792 - protein, 15 aa, Homo sapiens
RRTPSPDYDFYYRPR

SEQ ID NO: 793 - protein, 11 aa, Homo sapiens
RVKSPEPSHPK

SEQ ID NO: 794 - protein, 18 aa, Homo sapiens
SLSPTYIELMRPVSELIR

SEQ ID NO: 795 - protein, 22 aa, Homo sapiens
SRPQPAEEYEDDTERRSPTPER

SEQ ID NO: 796 - protein, 22 aa, Homo sapiens
SRPQPAEEYEDDTERRSPTPER

SEQ ID NO: 797 - protein, 12 aa, Homo sapiens
SRSPTPPSIAAK

SEQ ID NO: 798 - protein, 11 aa, Homo sapiens
VKSPEAVKSPK

SEQ ID NO: 799 - protein, 31 aa, Homo sapiens
NATQQKDMVEVADFDFSPMSDKNPEPPSGVR

SEQ ID NO: 800 - protein, 12 aa, Homo sapiens
GKSEEELSDLFR

SEQ ID NO: 801 - protein, 7 aa, Homo sapiens
RRSSNYR

SEQ ID NO: 802 - protein, 19 aa, Homo sapiens
ELWQSIYNLEAEKFDLQEK

SEQ ID NO: 803 - protein, 30 aa, Homo sapiens
LHSDSGISVDSQSLHDQQPHTQTASGQALK

SEQ ID NO: 804 - protein, 25 aa, Homo sapiens
VNFSEEGETEEDDQDSSHSSVTTVK

SEQ ID NO: 805 - protein, 24 aa, Homo sapiens
ASPAPGSGHPEGPGAHLDMNSLDR

SEQ ID NO: 806 - protein, 11 aa, Homo sapiens
NSPTFKSFEER

SEQ ID NO: 807 - protein, 11 aa, Homo sapiens
NSATFKSFEDR

SEQ ID NO: 808 - protein, 13 aa, Homo sapiens
IIYGGSVTGATCK

SEQ ID NO: 809 - protein, 15 aa, Homo sapiens
KQSLGELIGTLNAAK

SEQ ID NO: 810 - protein, 11 aa, Homo sapiens
KLVIIESDLER

SEQ ID NO: 811 - protein, 14 aa, Homo sapiens
LVIIESDLERAEER

SEQ ID NO: 812 - protein, 13 aa, Homo sapiens
SIDDLEDELYAQK

SEQ ID NO: 813 - protein, 16 aa, Homo sapiens
AISEELDHALNDMTSI

SEQ ID NO: 814 - protein, 16 aa, Homo sapiens
AISEELDHALNDMTSI

SEQ ID NO: 815 - protein, 14 aa, Homo sapiens
ATDAEADVASLNRR

SEQ ID NO: 816 - protein, 14 aa, Homo sapiens
KATDAEADVASLNR

SEQ ID NO: 817 - protein, 15 aa, Homo sapiens
KATDAEADVASLNRR

SEQ ID NO: 818 - protein, 7 aa, Homo sapiens
LATALQK

SEQ ID NO: 819 - protein, 16 aa, Homo sapiens
AISEELDNALNDITSL

SEQ ID NO: 820 - protein, 15 aa, Homo sapiens
AANRTPPKSPGDPSK

SEQ ID NO: 821 - protein, 9 aa, Homo sapiens
AISSPTVSR

SEQ ID NO: 822 - protein, 10 aa, Homo sapiens
ASAVSELSPR

SEQ ID NO: 823 - protein, 16 aa, Homo sapiens
ERSPALKSPLQSVVVR

SEQ ID NO: 824 - protein, 12 aa, Homo sapiens
GSFSDTGLGDGK

SEQ ID NO: 825 - protein, 9 aa, Homo sapiens
IDISPSTFR

SEQ ID NO: 826 - protein, 19 aa, Homo sapiens
MDSFDEDLARPSGLLAQER

SEQ ID NO: 827 - protein, 9 aa, Homo sapiens
NKKSPEIHR

SEQ ID NO: 828 - protein, 10 aa, Homo sapiens
RIDISPSTFR

SEQ ID NO: 829 - protein, 14 aa, Homo sapiens
SPPSTGSTYGSSQK

SEQ ID NO: 830 - protein, 19 aa, Homo sapiens
SPVGKSPPSTGSTYGSSQK

SEQ ID NO: 831 - protein, 33 aa, Homo sapiens
AHTPTPGIYMGRPTHSGGGGGGGGGGGGGGGGR

SEQ ID NO: 832 - protein, 10 aa, Homo sapiens
RRSPSPYYSR

SEQ ID NO: 833 - protein, 10 aa, Homo sapiens
RRSPSPYYSR

SEQ ID NO: 834 - protein, 12 aa, Homo sapiens
RHSHSHSPMSTR

SEQ ID NO: 835 - protein, 20 aa, Homo sapiens
RPHTPTPGIYMGRPTYGSSR

SEQ ID NO: 836 - protein, 39 aa, Homo sapiens
NINQVVKQRSLTPSPMNIPGSNQSSAMNSLLSSCVSTPR

SEQ ID NO: 837 - protein, 8 aa, Homo sapiens
LERETLQK

SEQ ID NO: 838 - protein, 41 aa, Homo sapiens
MMAALYPSTDLSGASSSSLPSSPSSSSPNEVMALKDVREVK

SEQ ID NO: 839 - protein, 6 aa, Homo sapiens
LKDSLR

SEQ ID NO: 840 - protein, 29 aa, Homo sapiens
NMSPSSGHQSPAGSAPSPALSYSSAGSAR

SEQ ID NO: 841 - protein, 20 aa, Homo sapiens
MASAGTQHYSIGLRQKNSFK

SEQ ID NO: 842 - protein, 16 aa, Homo sapiens
KVSKQEEASGGPTAPK

SEQ ID NO: 843 - protein, 20 aa, Homo sapiens
VNNSSLIGLGYTQTLKPGIK

SEQ ID NO: 844 - protein, 13 aa, Homo sapiens
LTFDTTFSPNTGK

SEQ ID NO: 845 - protein, 20 aa, Homo sapiens
VNNSSLIGVGYTQTLRPGVK

SEQ ID NO: 846 - protein, 9 aa, Homo sapiens
LRSSVPGVR

SEQ ID NO: 847 - protein, 14 aa, Homo sapiens
TYSLGSALRPSTSR

SEQ ID NO: 848 - protein, 13 aa, Homo sapiens
LCDDGPQLPTSPR

SEQ ID NO: 849 - protein, 21 aa, Homo sapiens
VHDRSEEEEEEEEEEEEEQPR

SEQ ID NO: 850 - protein, 24 aa, Homo sapiens
TTLFTQAAHGGQASLMKISDSTLK

SEQ ID NO: 851 - protein, 9 aa, Homo sapiens
GISPIVFDR

SEQ ID NO: 852 - protein, 23 aa, Homo sapiens
LSSESHHGGSPIHWVLPAGMSAK

SEQ ID NO: 853 - protein, 11 aa, Homo sapiens
SLSPSHLTEDR

SEQ ID NO: 854 - protein, 16 aa, Homo sapiens
SLGRSLGPIMASMADR

SEQ ID NO: 855 - protein, 9 aa, Homo sapiens
LGVSVSPSR

SEQ ID NO: 856 - protein, 40 aa, Homo sapiens
TGSSPNSVSSSPTNSAITQLRNKLENGKPLSMSEQTGLLK

SEQ ID NO: 857 - protein, 16 aa, Homo sapiens
CGKSFSQSTYLIEHQR

SEQ ID NO: 858 - protein, 37 aa, Homo sapiens
IGSGFLSGGGGTGSSGGSGSGGGGSGGGGGGGSSGRR

SEQ ID NO: 859 - protein, 9 aa, Homo sapiens
QFTTAGNLK

SEQ ID NO: 860 - protein, 17 aa, Homo sapiens
QCGKAFRSASLLQTHGR

SEQ ID NO: 861 - protein, 10 aa, Homo sapiens
RSFLQQDVNK

SEQ ID NO: 862 - protein, 8 aa, Homo sapiens
SLDDKPYK

SEQ ID NO: 863 - protein, 30 aa, Homo sapiens
DVMLETYGNLVSLGVGPAGPKPGVISQLER

SEQ ID NO: 864 - protein, 12 aa, Homo sapiens
DRPVGGSPGGPR

SEQ ID NO: 865 - protein, 20 aa, Homo sapiens
DKNYGSYYGVGDYPVVKLLR

SEQ ID NO: 866 - protein, 20 aa, Homo sapiens
ENVEYIEREESDGEYDEFGR

SEQ ID NO: 867 - protein, 20 aa, Homo sapiens
EVEDKESEGEEEDEDEDLSK

SEQ ID NO: 868 - protein, 14 aa, Homo sapiens
YNLDASEEEDSNKK

SEQ ID NO: 869 - protein, 28 aa, Homo sapiens
QSDLGESEPSNVTETLMGGVSLGPAFVK

SEQ ID NO: 870 - protein, 13 aa, Homo sapiens
TCVADESAENCDK

SEQ ID NO: 871 - protein, 14 aa, Homo sapiens
GILAADESTGSIAK

SEQ ID NO: 872 - protein, 14 aa, Homo sapiens
GILAADESTGSIAK

SEQ ID NO: 873 - protein, 15 aa, Homo sapiens
GILAADESTGSIAKR

SEQ ID NO: 874 - protein, 17 aa, Homo sapiens
ATEDEGSEQKIPEATNR

SEQ ID NO: 875 - protein, 18 aa, Homo sapiens
KATEDEGSEQKIPEATNR

SEQ ID NO: 876 - protein, 26 aa, Homo sapiens
KPEDVLDDDDAGSAPLKSSGQHQNDK

SEQ ID NO: 877 - protein, 18 aa, Homo sapiens
RKPEDVLDDDDAGSAPLK

SEQ ID NO: 878 - protein, 27 aa, Homo sapiens
RKPEDVLDDDDAGSAPLKSSGQHQNDK

SEQ ID NO: 879 - protein, 27 aa, Homo sapiens
RKPEDVLDDDDAGSAPLKSSGQHQNDK

SEQ ID NO: 880 - protein, 16 aa, Homo sapiens
RGSIGENQVEVMVEEK

SEQ ID NO: 881 - protein, 23 aa, Homo sapiens
TPDGNKSPAPKPSDLRPGDVSSK

SEQ ID NO: 882 - protein, 9 aa, Homo sapiens
ELSQVLTQR

SEQ ID NO: 883 - protein, 21 aa, Homo sapiens
KYDLLCLYYHEPVSSDKVTQK

SEQ ID NO: 884 - protein, 16 aa, Homo sapiens
ASSVTTFTGEPNTCPR

SEQ ID NO: 885 - protein, 11 aa, Homo sapiens
RPFFPFHSPSR

SEQ ID NO: 886 - protein, 11 aa, Homo sapiens
TDGQVSGEAIK

SEQ ID NO: 887 - protein, 9 aa, Homo sapiens
FGESEKCPR

SEQ ID NO: 888 - protein, 31 aa, Homo sapiens; identical to SEQ ID NO: 195

SEQ ID NO: 889 - protein, 31 aa, Homo sapiens; identical to SEQ ID NO: 195

SEQ ID NO: 890 - protein, 17 aa, Homo sapiens
SLESTNVTDKDGELYCK

SEQ ID NO: 891 - protein, 15 aa, Homo sapiens
KTGQAPGYSYTAANK

SEQ ID NO: 892 - protein, 15 aa, Homo sapiens
KTGQAPGYSYTAANK

SEQ ID NO: 893 - protein, 14 aa, Homo sapiens
TGQAPGYSYTAANK

SEQ ID NO: 894 - protein, 21 aa, Homo sapiens
TFGGAPGFPLGSPLSSPVFPR

SEQ ID NO: 895 - protein, 21 aa, Homo sapiens
TFGGAPGFPLGSPLSSPVFPR

SEQ ID NO: 896 - protein, 21 aa, Homo sapiens
TFGGAPGFPLGSPLSSPVFPR

SEQ ID NO: 897 - protein, 17 aa, Homo sapiens
VVPSFLPVDQGGSLVGR

SEQ ID NO: 898 - protein, 11 aa, Homo sapiens
WEEHRSLLSGR

SEQ ID NO: 899 - protein, 39 aa, Homo sapiens; identical to SEQ ID NO: 230

SEQ ID NO: 900 - protein, 25 aa, Homo sapiens
GNPEPPVSGEMDDNSLSPEACYECK

SEQ ID NO: 901 - protein, 12 aa, Homo sapiens
CDSSPDSAEDVR

SEQ ID NO: 902 - protein, 13 aa, Homo sapiens
CDSSPDSAEDVRK

SEQ ID NO: 903 - protein, 20 aa, Homo sapiens
HTFMGVVSLGSPSGEVSHPR

SEQ ID NO: 904 - protein, 20 aa, Homo sapiens
HTFMGVVSLGSPSGEVSHPR

SEQ ID NO: 905 - protein, 20 aa, Homo sapiens
HTFMGVVSLGSPSGEVSHPR

SEQ ID NO: 906 - protein, 7 aa, Homo sapiens
YISFEER

SEQ ID NO: 907 - protein, 26 aa, Homo sapiens
KMGAPESGLAEYLFDKHTLGDSDNES

SEQ ID NO: 908 - protein, 15 aa, Homo sapiens
GALQNIIPASTGAAK

SEQ ID NO: 909 - protein, 17 aa, Homo sapiens
IISNASCTTNCLAPLAK

SEQ ID NO: 910 - protein, 24 aa, Homo sapiens
VIHDNFGIVEGLMTTVHAITATQK

SEQ ID NO: 911 - protein, 24 aa, Homo sapiens
VIHDNFGIVEGLMTTVHAITATQK

SEQ ID NO: 912 - protein, 16 aa, Homo sapiens
SETAPAAPAAAPPAEK

SEQ ID NO: 913 - protein, 7 aa, Homo sapiens
STELLIR

SEQ ID NO: 914 - protein, 18 aa, Homo sapiens
IDASKNEEDEGHSNSSPR

SEQ ID NO: 915 - protein, 18 aa, Homo sapiens
IDASKNEEDEGHSNSSPR

SEQ ID NO: 916 - protein, 11 aa, Homo sapiens
TVNSTRETPPK

SEQ ID NO: 917 - protein, 20 aa, Homo sapiens
ESEDKPEIEDVGSDEEEEKK

SEQ ID NO: 918 - protein, 24 aa, Homo sapiens
ESEDKPEIEDVGSDEEEEKKDGDK

SEQ ID NO: 919 - protein, 24 aa, Homo sapiens
ESEDKPEIEDVGSDEEEEKKDGDK

SEQ ID NO: 920 - protein, 14 aa, Homo sapiens
IEDVGSDEEDDSGK

SEQ ID NO: 921 - protein, 16 aa, Homo sapiens
IEDVGSDEEDDSGKDK

SEQ ID NO: 922 - protein, 17 aa, Homo sapiens
IEDVGSDEEDDSGKDKK

SEQ ID NO: 923 - protein, 8 aa, Homo sapiens
GPSWDPFR

SEQ ID NO: 924 - protein, 15 aa, Homo sapiens
GPSWDPFRDWYPHSR

SEQ ID NO: 925 - protein, 10 aa, Homo sapiens
QLSSGVSEIR

SEQ ID NO: 926 - protein, 22 aa, Homo sapiens
AERSFHSSSSSSSSSTSSSASR

SEQ ID NO: 927 - protein, 22 aa, Homo sapiens
AERSFHSSSSSSSSSTSSSASR

SEQ ID NO: 928 - protein, 19 aa, Homo sapiens
SFHSSSSSSSSSTSSSASR

SEQ ID NO: 929 - protein, 19 aa, Homo sapiens
SFHSSSSSSSSSTSSSASR

SEQ ID NO: 930 - protein, 19 aa, Homo sapiens
SFHSSSSSSSSSTSSSASR

SEQ ID NO: 931 - protein, 37 aa, Homo sapiens; identical to SEQ ID NO: 325

SEQ ID NO: 932 - protein, 37 aa, Homo sapiens; identical to SEQ ID NO: 325

SEQ ID NO: 933 - protein, 38 aa, Homo sapiens; identical to SEQ ID NO: 327

SEQ ID NO: 934 - protein, 23 aa, Homo sapiens
KVNAEGSVDSVFSQVCTHLDALK

SEQ ID NO: 935 - protein, 22 aa, Homo sapiens
VNAEGSVDSVFSQVCTHLDALK

SEQ ID NO: 936 - protein, 13 aa, Homo sapiens
YGYTHLSTGDLLR

SEQ ID NO: 937 - protein, 12 aa, Homo sapiens
GQSIDDMIPAQK

SEQ ID NO: 938 - protein, 21 aa, Homo sapiens
GTGGVDTAAVGSVFDVSNADR

SEQ ID NO: 939 - protein, 22 aa, Homo sapiens
RGTGGVDTAAVGSVFDVSNADR

SEQ ID NO: 940 - protein, 22 aa, Homo sapiens
RGTGGVDTAAVGSVFDVSNADR

SEQ ID NO: 941 - protein, 16 aa, Homo sapiens
LGYILTCPSNLGTGLR

SEQ ID NO: 942 - protein, 19 aa, Homo sapiens
ETTCSKESNEELTESCETK

SEQ ID NO: 943 - protein, 20 aa, Homo sapiens
ETTCSKESNEELTESCETKK

SEQ ID NO: 944 - protein, 19 aa, Homo sapiens
ETTCSKESNEELTESCETK

SEQ ID NO: 945 - protein, 20 aa, Homo sapiens
ETTCSKESNEELTESCETKK

SEQ ID NO: 946 - protein, 33 aa, Homo sapiens; identical to SEQ ID NO: 413

SEQ ID NO: 947 - protein, 33 aa, Homo sapiens; identical to SEQ ID NO: 413

SEQ ID NO: 948 - protein, 15 aa, Homo sapiens
AEDGATPSPSNETPK

SEQ ID NO: 949 - protein, 16 aa, Homo sapiens
AEDGATPSPSNETPKK

SEQ ID NO: 950 - protein, 32 aa, Homo sapiens; identical to SEQ ID NO: 426

SEQ ID NO: 951 - protein, 18 aa, Homo sapiens
GEPAAAAAPEAGASPVEK

SEQ ID NO: 952 - protein, 7 aa, Homo sapiens
LSGFSFK

SEQ ID NO: 953 - protein, 8 aa, Homo sapiens
LSGFSFKK

SEQ ID NO: 954 - protein, 21 aa, Homo sapiens
AGGANSNVFSMFEQTQIQEFK

SEQ ID NO: 955 - protein, 21 aa, Homo sapiens
AGGANSNVFSMFEQTQIQEFK

SEQ ID NO: 956 - protein, 22 aa, Homo sapiens
RAGGANSNVFSMFEQTQIQEFK

SEQ ID NO: 957 - protein, 22 aa, Homo sapiens
RAGGANSNVFSMFEQTQIQEFK

SEQ ID NO: 958 - protein, 13 aa, Homo sapiens
DIMLEELSHLSNR

SEQ ID NO: 959 - protein, 17 aa, Homo sapiens
SPPNPDNIAPGYSGPLK

SEQ ID NO: 960 - protein, 24 aa, Homo sapiens
VDGSNLEGGSQQAPLTPPNTPDPR

SEQ ID NO: 961 - protein, 24 aa, Homo sapiens
VDGSNLEGGSQQAPLTPPNTPDPR

SEQ ID NO: 962 - protein, 24 aa, Homo sapiens
VDGSNLEGGSQQAPLTPPNTPDPR

SEQ ID NO: 963 - protein, 15 aa, Homo sapiens
KTGSYGALAEITASK

SEQ ID NO: 964 - protein, 15 aa, Homo sapiens
KTGSYGALAEITASK

SEQ ID NO: 965 - protein, 16 aa, Homo sapiens
RSTQGVTLTDLQEAEK

SEQ ID NO: 966 - protein, 16 aa, Homo sapiens
RSTQGVTLTDLQEAEK

SEQ ID NO: 967 - protein, 15 aa, Homo sapiens
SYLTPVRDEESESQR

SEQ ID NO: 968 - protein, 18 aa, Homo sapiens
DEDETDGSEEVKETWHER

SEQ ID NO: 969 - protein, 10 aa, Homo sapiens
SLDEEPICHR

SEQ ID NO: 970 - protein, 16 aa, Homo sapiens
AAFSKDESKEPIVEVR

SEQ ID NO: 971 - protein, 19 aa, Homo sapiens
TDPGSIFDLDPLEDNIQSR

SEQ ID NO: 972 - protein, 14 aa, Homo sapiens
EMLASDDEEDVSSK

SEQ ID NO: 973 - protein, 17 aa, Homo sapiens
EMLASDDEEDVSSKVEK

SEQ ID NO: 974 - protein, 11 aa, Homo sapiens
TISQEFLTPGK

SEQ ID NO: 975 - protein, 25 aa, Homo sapiens
EFITGDVEPTDAESEWHSENEEEEK

SEQ ID NO: 976 - protein, 31 aa, Homo sapiens; identical to SEQ ID NO: 484

SEQ ID NO: 977 - protein, 11 aa, Homo sapiens
RSSPPGHYYQK

SEQ ID NO: 978 - protein, 9 aa, Homo sapiens
YGMGTSVER

SEQ ID NO: 979 - protein, 14 aa, Homo sapiens
YHGHSMSDPGVSYR

SEQ ID NO: 980 - protein, 14 aa, Homo sapiens
YHGHSMSDPGVSYR

SEQ ID NO: 981 - protein, 14 aa, Homo sapiens
YHGHSMSDPGVSYR

SEQ ID NO: 982 - protein, 14 aa, Homo sapiens
YHGHSMSDPGVSYR

SEQ ID NO: 983 - protein, 14 aa, Homo sapiens
YHGHSMSDPGVSYR

SEQ ID NO: 984 - protein, 15 aa, Homo sapiens
NRPTSISWDGLDSGK

SEQ ID NO: 985 - protein, 15 aa, Homo sapiens
NRPTSISWDGLDSGK

SEQ ID NO: 986 - protein, 24 aa, Homo sapiens
LLKPGEEPSEYTDEEDTKDHNKQD

SEQ ID NO: 987 - protein, 18 aa, Homo sapiens
LLKPGEEPSEYTDEEDTK

SEQ ID NO: 988 - protein, 22 aa, Homo sapiens
LLKPGEEPSEYTDEEDTKDHNK

SEQ ID NO: 989 - protein, 24 aa, Homo sapiens
LLKPGEEPSEYTDEEDTKDHNKQD

SEQ ID NO: 990 - protein, 16 aa, Homo sapiens
GTSSMSSLHVSSPHQR

SEQ ID NO: 991 - protein, 16 aa, Homo sapiens
GTSSMSSLHVSSPHQR

SEQ ID NO: 992 - protein, 16 aa, Homo sapiens
GTSSMSSLHVSSPHQR

SEQ ID NO: 993 - protein, 24 aa, Homo sapiens
MKPIEEGAEDDDDVFEPASPNTLK

SEQ ID NO: 994 - protein, 24 aa, Homo sapiens
MKPIEEGAEDDDDVFEPASPNTLK

SEQ ID NO: 995 - protein, 18 aa, Homo sapiens
NSIASSSDSDDGLHQFLR

SEQ ID NO: 996 - protein, 18 aa, Homo sapiens
NSIASSSDSDDGLHQFLR

SEQ ID NO: 997 - protein, 18 aa, Homo sapiens
NSIASSSDSDDGLHQFLR

SEQ ID NO: 998 - protein, 44 aa, Homo sapiens; identical to SEQ ID NO: 568

SEQ ID NO: 999 - protein, 44 aa, Homo sapiens; identical to SEQ ID NO: 568

SEQ ID NO: 1000 - protein, 7 aa, Homo sapiens
KVSVNVK

SEQ ID NO: 1001 - protein, 9 aa, Homo sapiens
LPAKLSISK

SEQ ID NO: 1002 - protein, 24 aa, Homo sapiens
RGSSPDVHALLEITEESDAVLVDK

SEQ ID NO: 1003 - protein, 11 aa, Homo sapiens
SFTPDHVVYAR

SEQ ID NO: 1004 - protein, 11 aa, Homo sapiens
SLKESEALPEK

SEQ ID NO: 1005 - protein, 18 aa, Homo sapiens
VMIYQDEVKLPAKLSISK

SEQ ID NO: 1006 - protein, 18 aa, Homo sapiens
SHTEEDCTEELFDFLHAR

SEQ ID NO: 1007 - protein, 18 aa, Homo sapiens
SHTEEDCTEELFDFLHAR

SEQ ID NO: 1008 - protein, 17 aa, Homo sapiens
KEESEESDDDMGFGLFD

SEQ ID NO: 1009 - protein, 17 aa, Homo sapiens
KEESEESDDDMGFGLFD

SEQ ID NO: 1010 - protein, 22 aa, Homo sapiens
SSGSPYGGGYGSGGGSGGYGSR

SEQ ID NO: 1011 - protein, 44 aa, Homo sapiens; identical to SEQ ID NO: 622

SEQ ID NO: 1012 - protein, 18 aa, Homo sapiens
ISSGKSSPFKVSPLTFGR

SEQ ID NO: 1013 - protein, 18 aa, Homo sapiens
ISSGKSSPFKVSPLTFGR

SEQ ID NO: 1014 - protein, 13 aa, Homo sapiens
SSPFKVSPLTFGR

SEQ ID NO: 1015 - protein, 36 aa, Homo sapiens; identical to SEQ ID NO: 626

SEQ ID NO: 1016 - protein, 31 aa, Homo sapiens; identical to SEQ ID NO: 627

SEQ ID NO: 1017 - protein, 36 aa, Homo sapiens; identical to SEQ ID NO: 626

SEQ ID NO: 1018 - protein, 36 aa, Homo sapiens; identical to SEQ ID NO: 626

SEQ ID NO: 1019 - protein, 13 aa, Homo sapiens
SSPFKVSPLTFGR

SEQ ID NO: 1020 - protein, 8 aa, Homo sapiens
VSPLTFGR

SEQ ID NO: 1021 - protein, 21 aa, Homo sapiens
DASSPVPPPHVPPPVPPLRPR

SEQ ID NO: 1022 - protein, 21 aa, Homo sapiens
DASSPVPPPHVPPPVPPLRPR

SEQ ID NO: 1023 - protein, 18 aa, Homo sapiens
GAEDYPDPPIPHSYSSDR

SEQ ID NO: 1024 - protein, 11 aa, Homo sapiens
RKSEPAVGPPR

SEQ ID NO: 1025 - protein, 9 aa, Homo sapiens
SEPAVGPPR

SEQ ID NO: 1026 - protein, 12 aa, Homo sapiens
SFTSSSPSSPSR

SEQ ID NO: 1027 - protein, 12 aa, Homo sapiens
SFTSSSPSSPSR

SEQ ID NO: 1028 - protein, 11 aa, Homo sapiens
SHSDNSPNAFK

SEQ ID NO: 1029 - protein, 17 aa, Homo sapiens
TSPGRVDLPGSSTTLTK

SEQ ID NO: 1030 - protein, 17 aa, Homo sapiens
TSPGRVDLPGSSTTLTK

SEQ ID NO: 1031 - protein, 28 aa, Homo sapiens
AEVGAPLSPDHSEEEEEEEEGLEEDEPR

SEQ ID NO: 1032 - protein, 28 aa, Homo sapiens
AEVGAPLSPDHSEEEEEEEEGLEEDEPR

SEQ ID NO: 1033 - protein, 28 aa, Homo sapiens
AEVGAPLSPDHSEEEEEEEEGLEEDEPR

SEQ ID NO: 1034 - protein, 28 aa, Homo sapiens
DDSEEEKEKEEDPGSHEEDDESSEQGEK

SEQ ID NO: 1035 - protein, 12 aa, Homo sapiens
DEEEDEDVSTER

SEQ ID NO: 1036 - protein, 41 aa, Homo sapiens; identical to SEQ ID NO: 690

SEQ ID NO: 1037 - protein, 18 aa, Homo sapiens
EEDEEVSAELGHQAPSHR

SEQ ID NO: 1038 - protein, 21 aa, Homo sapiens
EKEEDPGSHEEDDESSEQGEK

SEQ ID NO: 1039 - protein, 32 aa, Homo sapiens; identical to SEQ ID NO: 29

SEQ ID NO: 1040 - protein, 22 aa, Homo sapiens
GHGSEEDEDVSDGHHHHGPSHR

SEQ ID NO: 1041 - protein, 14 aa, Homo sapiens
GHGSEDTEDSAEHR

SEQ ID NO: 1042 - protein, 14 aa, Homo sapiens
GHGSEDTEDSAEHR

SEQ ID NO: 1043 - protein, 14 aa, Homo sapiens
GHKSDEEDFQDEYK

SEQ ID NO: 1044 - protein, 28 aa, Homo sapiens
HQGHEEDDDDDDDDDDDDDDDDVSIEYR

SEQ ID NO: 1045 - protein, 17 aa, Homo sapiens
HQGHRDEEEDEDVSTER

SEQ ID NO: 1046 - protein, 24 aa, Homo sapiens
HRSHEEDDNDDDDVSTEYGHQAHR

SEQ ID NO: 1047 - protein, 24 aa, Homo sapiens
HRSHEEDDNDDDDVSTEYGHQAHR

SEQ ID NO: 1048 - protein, 22 aa, Homo sapiens
SHEEDDNDDDDVSTEYGHQAHR

SEQ ID NO: 1049 - protein, 20 aa, Homo sapiens
VGDEGVSGEEVFAEHGGQAR

SEQ ID NO: 1050 - protein, 21 aa, Homo sapiens
VPREEDEEVSAELGHQAPSHR

SEQ ID NO: 1051 - protein, 15 aa, Homo sapiens
DWEDDSDEDMSNFDR

SEQ ID NO: 1052 - protein, 27 aa, Homo sapiens
LNWLSVDFNNWKDWEDDSDEDMSNFDR

SEQ ID NO: 1053 - protein, 27 aa, Homo sapiens
LNWLSVDFNNWKDWEDDSDEDMSNFDR

SEQ ID NO: 1054 - protein, 27 aa, Homo sapiens
EEREDTPIQLQELLALETALGGQCVDR

SEQ ID NO: 1055 - protein, 9 aa, Homo sapiens
SMSQEAQRG

SEQ ID NO: 1056 - protein, 12 aa, Homo sapiens
GKSEEELSDLFR

SEQ ID NO: 1057 - protein, 7 aa, Homo sapiens
RRSSNYR

SEQ ID NO: 1058 - protein, 19 aa, Homo sapiens
ELWQSIYNLEAEKFDLQEK

SEQ ID NO: 1059 - protein, 13 aa, Homo sapiens
IIYGGSVTGATCK

SEQ ID NO: 1060 - protein, 15 aa, Homo sapiens
KQSLGELIGTLNAAK

SEQ ID NO: 1061 - protein, 11 aa, Homo sapiens
KLVIIESDLER

SEQ ID NO: 1062 - protein, 14 aa, Homo sapiens
LVIIESDLERAEER

SEQ ID NO: 1063 - protein, 13 aa, Homo sapiens
SIDDLEDELYAQK

SEQ ID NO: 1064 - protein, 16 aa, Homo sapiens
AISEELDHALNDMTSI

SEQ ID NO: 1065 - protein, 16 aa, Homo sapiens
AISEELDHALNDMTSI

SEQ ID NO: 1066 - protein, 14 aa, Homo sapiens
ATDAEADVASLNRR

SEQ ID NO: 1067 - protein, 14 aa, Homo sapiens
KATDAEADVASLNR

SEQ ID NO: 1068 - protein, 15 aa, Homo sapiens
KATDAEADVASLNRR

SEQ ID NO: 1069 - protein, 7 aa, Homo sapiens
LATALQK

SEQ ID NO: 1070 - protein, 16 aa, Homo sapiens
AISEELDNALNDITSL

SEQ ID NO: 1071 - protein, 15 aa, Homo sapiens
AANRTPPKSPGDPSK

SEQ ID NO: 1072 - protein, 9 aa, Homo sapiens
AISSPTVSR

SEQ ID NO: 1073 - protein, 20 aa, Homo sapiens
VNNSSLIGLGYTQTLKPGIK

SEQ ID NO: 1074 - protein, 13 aa, Homo sapiens
LTFDTTFSPNTGK

SEQ ID NO: 1075 - protein, 20 aa, Homo sapiens
VNNSSLIGVGYTQTLRPGVK

SEQ ID NO: 1076 - protein, 9 aa, Homo sapiens
LRSSVPGVR

SEQ ID NO: 1077 - protein, 14 aa, Homo sapiens
TYSLGSALRPSTSR

SEQ ID NO: 1078 - protein, 19 aa, Homo sapiens
  MISC_FEATURE (16)..(16): Phosphorylation site
TFMGVVSLGSPSGEVSHPR

SEQ ID NO: 1079 - protein, 458 aa, Homo sapiens [sequence omitted]

SEQ ID NO: 1080 - protein, 582 aa, Homo sapiens
MAARSWQDELAQQAEEGSARLREMLSVGLGFLRTELGLDLGLEPKRYPGWVILVGTGALGLLLLFLLGYGWAAACAGARKKRRSPPRKREEAAAVPAAAPDDLALLKNLRSEEQKKKNRKKLSEKPKPNGRTVEVAEGEAVRTPQSVTAKQPPEIDKKNEKSKKNKKKSKSDAKAVQNSSRHDGKEVDEGAWETKISHREKRQQRKRDKVLTDSGSLDSTIPGIENTITVTTEQLTTASFPVGSKKNKGDSHLNVQVSNFKSGKGDSTLQVSSGLNENLTVNGGGWNEKSVKLSSQISAGEEKWNSVSPASAGKRKTEPSAWSQDTGDANTNGKDWGRSWSDRSIFSGIGSTAEPVSQSTTSDYQWDVSRNQPYIDDEWSGLNGLSSADPNSDWNAPAEEWGNWVDEERASLLKSQEPIPDDQKVSDDDKEKGEGALPTGKSKKKKKKKKKQGEDNSTAQDTEELEKEIREDLPVNTSKTRPKQEKAFSLKTISTSDPAEVLVKNSQPIKTLPPATSTEPSVILSKSDSDKSSSQVPPILQETDKSKSNTKQNSVPPSQTKSETSWESPKQIKKKKKARRET

SEQ ID NO: 1081 - protein, 600 aa, Homo sapiens
MSRVAKYRRQVSEDPDIDSLLETLSPEEMEELEKELDVVDPDGSVPVGLRQRNQTEKQSTGVYNREAMLNFCEKETKKLMQREMSMDESKQVETKTDAKNGEERGRDASKKALGPRRDSDLGKEPKRGGLKKSFSRDRDEAGGKSGEKPKEEKIIRGIDKGRVRAAVDKKEAGKDGRGEERAVATKKEEEKKGSDRNTGLSRDKDKKREEMKEVAKKEDDEKVKGERRNTDTRKEGEKMKRAGGNTDMKKEDEKVKRGTGNTDTKKDDEKVKKNEPLHEKEAKDDSKTKTPEKQTPSGPTKPSEGPAKVEEEAAPSIFDEPLERVKNNDPEMTEVNVNNSDCITNEILVRFTEALEFNTVVKLFALANTRADDHVAFAIAIMLKANKTITSLNLDSNHITGKGILAIFRALLQNNTLTELRFHNQRHICGGKTEMEIAKLLKENTTLLKLGYHFELAGPRMTVTNLLSRNMDKQRQKRLQEQRQAQEAKGEKKDLLEVPKAGAVAKGSPKPSPQPSPKPSPKNSPKKGGAPAAPPPPPPPLAPPLIMENLKNSLSPATQRKMGDKVLPAQEKNSRDQLLAAIRSSNLKQLKKVEVPKLLQ

SEQ ID NO: 1082 - protein, 385 aa, Homo sapiens
MAAALFVLLGFALLGTHGASGAAGFVQAPLSQQRWVGGSVELHCEAVGSPVPEIQWWFEGQGPNDTCSQLWDGARLDRVHIHATYHQHAASTISIDTLVEEDTGTYECRASNDPDRNHLTRAPRVKWVRAQAVVLVLEPGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSHLAALWPFLGIVAEVLVLVTIIFIYEKRRKPEDVLDDDDAGSAPLKSSGQHQNDKGKNVRQRNSS

SEQ ID NO: 1083 - protein, 166 aa, Homo sapiens
MAPKKAKKRAGGANSNVFSMFEQTQIQEFKEAFTIMDQNRDGFIDKNDLRDTFAALGRVNVKNEEIDEMIKEAPGPINFTVFLTMFGEKLKGADPEETILNAFKVFDPEGKGVLKADYVREMLTTQAERFSKEEVDQMFAAFPPDVTGNLDYKNLVHIITHGEEKD

SEQ ID NO: 1084 - protein, 390 aa, Homo sapiens
MRKMLAAVSRVLSGASQKPASRVLVASRNFANDATFEIKKCDLHRLEEGPPVTTVLTREDGLKYYRMMQTVRRMELKADQLYKQKIIRGFCHLCDGQEACCVGLEAGINPTDHLITAYRAHGFTFTRGLSVREILAELTGRKGGCAKGKGGSMHMYAKNFYGGNGIVGAQVPLGAGIALACKYNGKDEVCLTLYGDGAANQGQIFEAYNMAALWKLPCIFICENNRYGMGTSVERAAASTDYYKRGDFIPGLRVDGMDILCVREATRFAAAYCRSGKGPILMELQTYRYHGHSMSDPGVSYRTREEIQEVRSKSDPIMLLKDRMVNSNLASVEELKEIDVEVRKEIEDAAQFATADPEPPLEELGYHIYSSDPPFEVRGANQWIKFKSVS